(19) United States
(12) Patent Application Publication
(10) Pub. No.: US 2026/0207602 A1
(43) Pub. Date: Jul. 23, 2026

(54) EGFR INHIBITORS

(71) Applicant: Blueprint Medicines Corporation, Cambridge, MA (US)

(72) Inventors: Omar Ahmad, Cambridge, MA (US); John Emmerson Campbell, Cambridge, MA (US); Thomas A. Dineen, Cambridge, MA (US); Meredith Suzanne Eno, Cambridge, MA (US); Dilinie Prasadhini Fernando, Cambridge, MA (US); Chandrasekhar V. Miduturu, Cambridge, MA (US); Emanuele Perola, Cambridge, MA (US)

(21) Appl. No.: 19/110,923

(22) PCT Filed: Sep. 14, 2023
(86) PCT No.: PCT/US2023/032689

§ 371 Date: Mar. 12, 2025

Related U.S. Application Data
(60) Provisional application No. 63/406,386, filed on Sep. 14, 2022.

(51) Int. Cl. A61K 31/517; A61K 31/506; A61K 31/5377; A61K 31/5386; C07B 59/00; C07D 417/14; C07D 487/04; C07D 491/107; C07D 498/08

(52) U.S. Cl. CPC A61K 31/517; A61K 31/506; A61K 31/5377; A61K 31/5386; C07B 59/002; C07D 417/14; C07D 487/04; C07D 491/107; C07D 498/08

(57) ABSTRACT

The present disclosure provides a compound represented by structural formula (I): or a pharmaceutically acceptable salt thereof useful for treating a cancer.

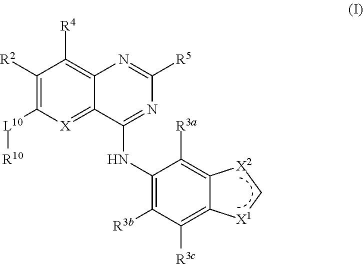

### CROSS-REFERENCE TO RELATED APPLICATIONS

This application claims priority to U.S. Provisional Application No. 63/406,386, filed Sep. 14, 2022. The entire contents of the aforementioned application is incorporated herein by reference.

### BACKGROUND

EGFR (Epidermal Growth Factor Receptor) is a member of the erbB receptor family, which includes transmembrane protein tyrosine kinase receptors. By binding to its ligand, such as epidermal growth factor (EGF), EGFR can form a homodimer on the cell membrane or form a heterodimer with other receptors in the family, such as erbB2, erbB3, or erbB4. The formation of these dimers can cause the phosphorylation of key tyrosine residues in EGFR cells, thereby activating a number of downstream signaling pathways in cells. These intracellular signaling pathways play an important role in cell proliferation, survival and anti-apoptosis. Disorders of EGFR signal transduction pathways, including increased expression of ligands and receptors, EGFR gene amplification and alterations such as mutations, deletions and the like, can promote malignant transformation of cells and play an important role in tumor cell proliferation, invasion, metastasis and angiogenesis. For example, alterations such as mutations and deletions in the EGFR gene are found in non-small lung cancer (NSCLC) tumors. The two most frequent EGFR alternations found in NSCLC tumors are short in-frame deletions in exon 19 (del19) and L858R, a single missense mutation in exon 21 (Cancer Discovery 2016 6(6) 601). These two alterations, referred to as sensitizing mutations, cause ligand-independent EGFR activation and are referred to as primary or activating mutations in EGFR mutant NSCLC (EGFR M+). Clinical experience shows an objective response rate (ORR) of approximately 60-85% in EGFR M+ NSCLC patients treated first line (1L) with EGFR tyrosine kinase inhibitors (TKIs) erlotinib, gefitinib, afatinib and osimertinib (Lancet Oncol. 2010 Vol. 11, 121; Lancet Oncol. 2016 Vol. 17, 577; N. Engl. J. Med. 2017 Nov. 18 Doi:10.1056/NEJMoa1713137; Lancet Oncol. 2011 Vol. 12, 735), thus demonstrating that EGFR mutant NSCLC tumors depend on oncogenic EGFR activity for survival and proliferation and establishing del19 and L858R mutated EGFR as oncogenic drivers of disease and thus, validating drug targets and biomarkers for the treatment of NSCLC.

Osimertinib is a covalent third (3^{rd}) generation EGFR TKI that is now the approved standard of care (SOC) in first line (1L) for the treatment of NSCLC harboring del19 and L858R mutations. With a progression-free survival (PFS) of 18.9 mo (J C Soria et al—NEJM, 2018 January; 378(2):113-125), it shows a transformative outcome for patients compared to first generation TKIs. However, after an average of 10-12 months of treatment, resistance has been observed in almost all NSCLC patients (Lancet Oncol. 2010 February; 11(2):121-8; Lancet Oncol. 2016 May; 17(5):577-89; Lancet Oncol. 2011 August; 12(8):735-42). Additional 3^{rd} generation TKIs are being used in front line (e.g. lazertinib) and relay on the same covalent mechanism of binding to EGFR. The most prominent on-target resistance mechanism is due to the secondary mutation in EGFR of C797X (where “X” can be an “S” or a “G” or an “N” or a “Y” or a “T” or a “D”), which occurs in 7% to 22% of patients progressing on 3rd generation EGFR inhibitors used in front line (Blakely, 2012; Kobayashi, 2005). This secondary C797S mutation reduces the affinity of the drug with the target, thereby producing drug resistance, and resulting in tumor recurrence or disease progression. The resulting “double mutant” tumors, that harbors the sensitizing mutations del19 or L858R and the resistance mutation C797X (e.g., C797S), are no longer sensitive to 2^{nd} and 3^{rd} generation TKIs. There is no approved drug to treat the double mutant patients. 1^{st} generation TKIs (gefitinib and erlotinib) are active against C797X (e.g., C797S) but they are poorly tolerated due to activity associated with wild-type EGFR inhibition, and do not control brain disease due to their low ability to cross the blood brain barrier (BBB).

There is an unmet need for a selective therapeutic agent that treats the double mutant tumors, that is brain penetrant and treats the brain disease, and with reduced toxicologies (diarrhea, skin rash) associated with wild-type EGFR inhibition.

### SUMMARY

The applicant has discovered novel compounds which are effective inhibitors of certain mutant forms of EGFR (see Synthetic Examples 1-415). In particular, it has been demonstrated that the compounds of the present disclosure effectively inhibit certain mutant forms of EGFR. Compounds of the disclosure (also referred to herein as the “disclosed compounds”) or pharmaceutically acceptable salts thereof effectively inhibit EGFR with one or more alterations, including L858R or exon 19 deletion mutation, and C797X (e.g., C797S) mutation (hereinafter “EGFR with LRCS mutations” or “double mutant EGFR”) (see Biological Example 1) and can be used treat various cancers, for example, lung cancer (see Biological Example 2). Importantly, the disclosed compounds are selective EGFR inhibitors, i.e., the disclosed compounds have no or low activity against wild-type EGFR and the kinome. Advantages associated with such selectivity may include facilitating efficacious dosing and reducing EGFR-mediated on-target toxicities. Some of the disclosed compounds exhibit good penetration of the brain and blood brain barrier (e.g., a PGP efflux ratio of less than 5). As such, the compounds of the disclosure or pharmaceutically acceptable salts thereof are expected to be effective for the treatment of metastatic cancer, including brain metastesis, including leptomeningeal disease and other systemic metastesis. Some of the disclosed compounds also have the advantage of having high microsomal stability. Compounds of the disclosure also may have favorable toxicity profiles related to other non-kinase targets.

In one aspect, the present disclosure provides a compound represented by the following structural Formula (I):

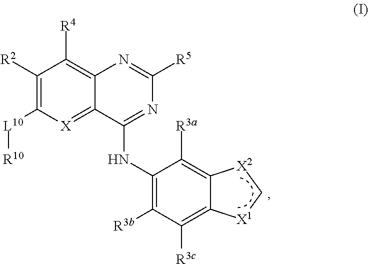

or a pharmaceutically acceptable salt thereof, the definition of each variable is provided below.

In another aspect, the present disclosure provides a pharmaceutical composition comprising a pharmaceutically acceptable carrier or diluent and one or more of the compounds disclosed herein, or a pharmaceutically acceptable salt thereof (a “pharmaceutical composition of the disclosure”).

The present disclosure provides a method of treating a subject with cancer, comprising administering to the subject an effective amount of a compound of the disclosure (e.g., a compound of Formula (I)) or a pharmaceutically acceptable salt thereof or a pharmaceutical composition of the disclosure. In one embodiment, the cancer is non-small cell lung cancer. In another embodiment, the subject cancer has metastasized to the brain. In another embodiment, the subject has brain metastasis from non-small cell lung cancer.

In one embodiment, the cancer to be treated has epidermal growth factor receptor (EGFR) L858R mutation or exon 19 deletion mutation. In another embodiment, the cancer to be treated may further has epidermal growth factor receptor (EGFR) L858R mutation or exon 19 deletion mutation and the C797X (e.g., C797S) mutation. In another embodiment, the cancer to be treated in either of the foregoing embodiments is lung cancer, e.g., non-small cell lung cancer. In a specific embodiment, the cancer is non-small cell lung cancer with brain metastasis or leptomeningeal disease.

The treatment method disclosed herein further comprises administering to the subject an effective amount of an EGFR inhibitor (e.g., afatinib and/or osimertinib), and a MET inhibitor in combination with an effective amount of a compound of the disclosure.

The present disclosure also provides a method of inhibiting epidermal growth factor receptor (EGFR) in a subject in need thereof, comprising administering to the subject an effective amount of a compound of the disclosure (e.g., a compound of Formula (I)) or a pharmaceutically acceptable salt thereof or a pharmaceutical composition of the disclosure.

The present disclosure also provides the use of an effective amount of a compound of the disclosure (e.g., a compound of Formula (I)), or a pharmaceutically acceptable salt thereof, or a pharmaceutical composition of the disclosure, for the preparation of a medicament for the treatment of cancers.

In another aspect, provided herein a compound of Formula (I), or a pharmaceutically acceptable salt thereof, or a pharmaceutical composition of the disclosure for use in treating cancers.

### DETAILED DESCRIPTION

### Definitions

The term “halo” as used herein means halogen and includes chloro, fluoro, bromo and iodo.

The term “alkyl” used alone or as part of a larger moiety, such as “alkoxy” and the like, means saturated aliphatic straight-chain or branched monovalent hydrocarbon radical. Unless otherwise specified, an alkyl group typically has 1-6 carbon atoms, i.e. (C₁-C₆)alkyl. As used herein, a “(C₁-C₆)alkyl” group means a radical having from 1 to 6 carbon atoms in a linear or branched arrangement. Examples include methyl, ethyl, n-propyl, iso-propyl, and the like.

The term “haloalkyl” or “C₁₋₄haloalkyl” refers to an alkyl group wherein at least one of the hydrogen atoms is replaced by a halo atom. The C₁₋₄haloalkyl group can be monohalo-C₁₋₄alkyl, dihalo-C₁₋₄alkyl or polyhalo-C₁₋₄ alkyl including perhalo-C₁₋₄alkyl. A monohalo-C₁₋₄alkyl can have one iodo, bromo, chloro or fluoro within the alkyl group. Dihalo-C₁₋₄alkyl and polyhalo-C₁₋₄alkyl groups can have two or more of the same halo atoms or a combination of different halo groups within the alkyl. Typically the polyhalo-C₁₋₄alkyl group contains up to 9, or 8, or 7, or 6, or 5, or 4, or 3, or 2 halo groups. Non-limiting examples of C₁₋₄haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. A perhalo-C₁₋₄alkyl group refers to a C₁₋₄alkyl group having all hydrogen atoms replaced with halo atoms.

The term “alkoxy” means an alkyl radical attached through an oxygen linking atom, represented by —O-alkyl. For example, “(C₁-C₄)alkoxy” includes methoxy, ethoxy, propoxy, and butoxy.

The term “cycloalkyl” refers to a monocyclic or bicyclic or polycyclic saturated hydrocarbon ring system. Cycloalkyl may include fused and/or bridged rings and/or spirocyclic rings. Non-limiting examples of fused/bridged cycloalkyl include: bicyclo[1.1.0]butane, bicyclo[2.1.0]pentane, bicyclo[1.1.0]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings share one ring atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like. Unless otherwise specified, cycloalkyl has from 3-12 carbon atoms. For example, a C₃₋C₆ cycloalkyl includes cyclopropyl, cyclobutyl, cyclopentyl and cyclohexyl. Unless otherwise described, a “cycloalkyl” has from three to six carbon atoms.

The term “heterocyclyl” or “heterocyclic” refers to a radical of a 4- to 12-membered non-aromatic ring system having ring carbon atoms and 1 to 4 ring heteroatoms, wherein each heteroatom is independently selected from nitrogen, quaternary nitrogen, oxidized nitrogen (e.g., NO), oxygen, and sulfur, including sulfoxide and sulfone (“4-12 membered heterocyclyl”). In some embodiments, a heterocyclyl group is a 4- to 8-membered non-aromatic ring system having ring carbon atoms and 1-4 (typically 1 to 2) ring heteroatoms, wherein each heteroatom is independently selected from nitrogen, oxygen, and sulfur (“4-8 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a bicyclic system (“bicyclic heterocyclyl”) or a tricyclic system (“tricyclic heterocyclyl”)). A polycyclic ring system includes fused, bridged, or spiro ring systems. When a heterocyclyl group is a polycyclic ring system, said ring system includes at least one non-aromatic ring. Exemplary monocyclic heterocyclyl groups include azetidinyl, oxetanyl, thietanyl, tetrahydrofuranyl, pyrrolidinyl, pyrrolidin-2-onyl, piperidinyl, tetrahydropyranyl, piperazinyl, morpholinyl, azepanyl, oxepanyl, thiepanyl, tetrahydropyridinyl, and the like. Heterocyclyl polycyclic ring systems can include heteroatoms in one or more rings in the polycyclic ring system-including polycyclic ring systems having a non-aromatic ring fused to a phenyl or heteroaryl ring. Exemplary polycyclic heterocyclic groups include 2H-benzo[b][1,4]oxazin-3(4H)-onyl, isoindolin-1-onyl, isoquinolin-1(2H)-onyl, 3-oxabicyclo[3.1.0]hexanyl, 8-oxa-3-azabicyclo[3.2.1]octanyl, 2-oxa-6-azaspiro[3.3]heptanyl, 6-oxa-3-azabicyclo[3.1.1]heptanyl, tetrahydropyrazolo[1,5-a]pyridinyl, and the like. Substituents may be present on one or more rings in the polycyclic ring system.

“Heteroaryl” refers to a radical of a 4- to 12-membered aromatic ring system having ring carbon atoms and 1 to 4 ring heteroatoms, wherein each heteroatom is independently selected from nitrogen, oxygen, and sulfur. In some embodiments, a heteroaryl group is a 5 or 6 membered heteroaryl having ring carbon atoms and 1 to 4 ring heteroatoms (typically 1 to 2). Representative heteroaryl groups include ring systems where each ring comprises a heteroatom and is aromatic, e.g., imidazolyl, oxazolyl, thiazolyl, triazolyl, pyrrolyl, furanyl, thiophenyl pyrazolyl, pyridinyl, pyrazinyl, pyridazinyl, pyrimidinyl, indolizinyl, purinyl, naphthyridinyl, and pteridinyl.

A bridged bicyclic system has two non-aromatic rings containing from 5-12 ring atoms (heterocyclyl or cycloalkyl) and which share three or more ring atoms, with the two bridgehead ring atoms separated by a bridge containing at least one atom. “Bridged heterocyclyl” includes bicyclic or polycyclic hydrocarbon or aza-bridged hydrocarbon groups; examples include bicyclo[1.1.1]pentanyl, 3-oxabicyclo[3.1.0]hexanyl, 2-azabicyclo[2.2.1]heptanyl, 6-oxa-3-azabicyclo[3.1.1]heptanyl, 3-azabicyclo[3.2.1]octanyl, 6-oxa-2-azabicyclo[3.2.1]octanyl, 6-oxa-3-azabicyclo[3.2.1]octanyl, and 8-oxa-3-azabicyclo[3.2.1]octanyl.

A fused bicyclic system has two rings containing from 6-12 ring atoms and which share two adjacent ring atoms. When the fused bicyclic system is heterocyclyl, at least one of the rings is non-aromatic. Examples of fused bicyclic systems include hexahydro-1H-furo[3,4-b]pyrrolyl, and hexahydro-1H-furo[3,4-c]pyrrolyl.

A spiro bicyclic system has two non-aromatic rings containing (heterocyclyl or cycloalkyl) from 7-12 ring atoms and which share one ring atom. Examples of spiro bicyclic systems include 1-oxa-7-azaspiro[3.5]nonan-7-yl, 2-oxa-6-azaspiro[3.3]heptanyl, 1,4-dioxa-8-azaspiro[4.5]decan-8-yl, and 1,4-dioxa-9-azaspiro[5.5]undecan-9-yl.

### Compounds of the Present Disclosure

Disclosed herein are embodiments of compounds having a general structure of Formula (I). These compounds are selective inhibitors of L858R, Ex19del, L858RC797S and Ex19DelC797S EGFR. In contrast to other EGFR inhibitors such as osimertinib which binds EGFR irreversibly, the compounds of the disclosure are non-covalent inhibitors.

In a first embodiment, the present disclosure provides a compound represented by the following structural formula (I):

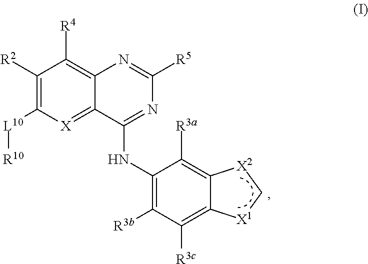

or a pharmaceutically acceptable salt thereof, wherein
X¹ is S and X² is N or X¹ is N and X² is S;
X is CR^{x} or N;
R^{x} is H, F, or —O—R¹;
R¹ is C₁-C₆ alkyl, C₃-C₆ cycloalkyl, or 4- to 12-membered heterocyclyl, wherein the alkyl, cycloalkyl, and heterocyclyl represented by R¹ are optionally substituted with 1 to 4 groups independently selected from deuterium, halo, C₁-C₄ alkyl, ═O (as valence permits), —OR^{1c}, CN, NR^{1a}R^{1b}, C₃-C₆ cycloalkyl, and 4- to 8-membered heterocyclyl, wherein the alkyl is optionally substituted with 1 to 3 groups selected from halo, deuterium, and OR^{1a} and NR^{1a}R^{1b}, and the heterocyclyl and C₃-C₆ cycloalkyl are each optionally substituted with 1 to 4 groups selected from ═O, NR^{1a}R^{1b}, and C₁-C₄ alkyl optionally substituted with NR^{1a}R^{1b};
L¹⁰-R¹⁰ is halo; or
L¹⁰ is a bond, NH, —NHC(O)—*, —NHC(O)O—*, O, or —OC(O)—*; wherein —* represents the point which attaches to R¹⁰; and
R¹⁰ is H; or
   C₁-C₄ alkyl optionally substituted with 1 to 4 groups independently selected from halo, deuterium, OR^{1a}, NR^{1a}R^{1b}, C₃-C₆ cycloalkyl, 4- to 12-membered heterocyclyl and 5 to 10 membered heteroaryl, wherein the heterocyclyl and heteroaryl are each optionally substituted with 1 to 4 groups independently selected from halo, deuterium, ═O, NR^{1a}R^{1b}, and C₁-C₄ alkyl; or
   C₃-C₈ cycloalkyl, phenyl, 4- to 12-membered heterocyclyl, or 5- to 12-membered heteroaryl, wherein the cycloalkyl, phenyl, heterocyclyl and heteroaryl represented by R¹⁰ are each optionally substituted with 1 to 4 groups independently selected from R¹¹;
each R¹¹ is independently selected from halo, deuterium, OR^{1a}, C(O)R^{1a}, C(O)NR^{1a}R^{1b}, NR^{1a}C(O)OR^{1a}, NR^{1a}R^{1b}, S(O)₂R^{1a}, C₁-C₄ alkyl, C₃-C₆ cycloalkyl, phenyl, 4- to 12-membered heterocyclyl and 5- to 12-membered heteroaryl, wherein the alkyl, cycloalkyl, phenyl, heterocyclyl and heteroaryl represented by R¹¹ are each optionally substituted with 1 to 4 groups selected from deuterium, halo, C₁-C₄ alkyl, OR^{1a} and NR^{1a}R^{1b}, or two R¹¹ which are attached to the same carbon atom are taken together to form ═O;
R² is halo, NR^{1a}R^{1b}, C₁-C₄ alkyl, C₂-C₄ alkenyl, C₂-C₄ alkynyl, C₁-C₄ alkoxy,
   C₂-C₄ alkenyloxy, C₂-C₄ alkynyloxy, C₃-C₈ cycloalkyl, 4- to 12-membered heterocyclyl, phenyl, or 5- or 12-membered heteroaryl, wherein the alkyl, alkenyl, alkynyl, alkoxy, alkenyloxy, alkynyloxy, cycloalkyl, heterocycyl, phenyl, and heteroaryl represented by R² are each optionally substituted with 1 to 4 groups represented by R^{2a}; and R^{2a} is selected from deuterium, halo, ═O (as valence permits), OR^{1c}, NR^{1a}R^{1b}, C(O)R^{1c}, C(O)OR^{1c}, —S(O)₂R^{1c}, C₁-C₄ alkyl,
   and 4- to 12-membered heterocyclyl, wherein the C₁-C₄ alkyl and C₁-C₄ alkoxy groups represented by R^{2a} are each optionally substituted with 1 to 4 groups selected from deuterium, halo, OH, and C₁-C₄ alkoxy, and the 4- to 12-membered heterocyclyl represented by R^{2a} is optionally substituted with 1 to 4 groups independently selected from halo, deuterium, ═O, NR^{1a}R^{1b}, and C₁-C₄ alkyl;
R^{1a} is H, deuterium, C₁-C₄ alkyl, or C₃-C₆ cycloalkyl;
R^{1b} is H, deuterium, C₁-C₄alkyl, or C₃-C₆cycloalkyl;
R^{1c} is H, deuterium, C₁-C₄ alkyl optionally substituted with 1 to 3 halo, or C₃-C₆ cycloalkyl;
R^{3a} is H, deuterium, halo, OH, C₁₋₄alkyl, or C₁-C₄ alkoxy;
R^{3b} is H, deuterium, halo, OH, C₁₋₄ alkyl, or C₁-C₄ alkoxy;
R^{3c} is H, deuterium, halo, OH, C₁₋₄alkyl, or C₁-C₄ alkoxy;
R⁴ is H, deuterium or halo; and
R⁵ is H or deuterium.

In a second embodiment, the compound according to Formula I is represented by Formula (II):

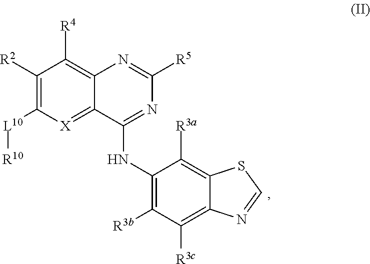

or a pharmaceutically acceptable salt thereof, wherein the remaining variables are as defined in the first embodiment.

In a third embodiment, the compound of Formula II is represented by Formula (III):

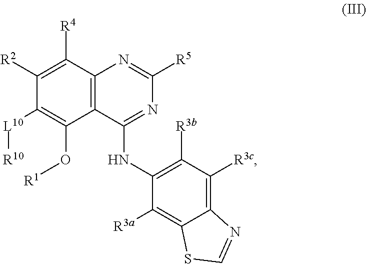

or a pharmaceutically acceptable salt thereof, wherein L¹⁰-R¹⁰ is H or halo; R^{3a} is H, deuterium, or halo; R^{3b} is H, deuterium, or halo; and R^{3c} is H, deuterium, or halo, wherein the remaining variables are as defined in the first embodiment.

In a fourth embodiment, the compound of Formula III is represented by Formula (III-A):

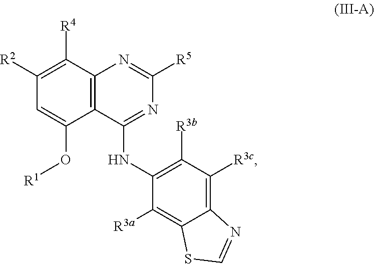

or a pharmaceutically acceptable salt thereof, wherein the remaining variables are as defined in the first embodiment.

In a fifth embodiment, the present disclosure provides a compound of Formula (I), (II), (III), or (III-A), or a pharmaceutically acceptable salt thereof, wherein R¹ is C₁-C₆ alkyl, C₃-C₆ cycloalkyl, or 4- to 8-membered heterocyclyl, wherein the alkyl, cycloalkyl, and heterocyclyl represented by R¹ are optionally substituted with 1 to 4 groups independently selected from halo, CN, ═O, NR^{1a}R^{1b}, C₁-C₄ alkyl (optionally substituted with 1 to 4 groups selected from halo, —OH and C₁-C₄ alkoxy), —OH, C₁-C₄ alkoxy, NR^{1a}R^{1b}, C₃-C₆ cycloalkyl, and 4- to 8-membered heterocyclyl (optionally substituted with ═O, NR^{1a}R^{1b}, and/or C₁-C₄ alkyl), wherein the remaining variables are as defined in the first, second, third or fourth embodiment.

In a sixth embodiment, the present disclosure provides a compound of Formula (I), (II), (III), or (III-A), or a pharmaceutically acceptable salt thereof, wherein R¹ is: C₁-C₆ alkyl optionally substituted with 1 or 4 groups selected from halo, CN, ═O, NHCH₃, N(CH₃)₂, OH, C₁-C₄ alkoxy, C₃-C₄ cycloalkyl, and 4- to 8-membered heterocyclyl (optionally substituted with 1 to 4 groups selected from ═O, NR^{1a}R^{1b}, and/or C₁-C₄ alkyl optionally substituted with NR^{1a}R^{1b}); C₃-C₆ cycloalkyl optionally substituted with 1 or 2 groups selected from C₁-C₄ alkyl, OH, NH₂, NHCH₃, and N(CH₃)₂; 4- to 6-membered heterocyclyl optionally substituted with 1 or 3 groups selected from halo, C₁-C₄ alkyl (optionally substituted with 1 to 4 groups selected from halo, —OH and C₁-C₄ alkoxy), ═O, OH, C₁-C₄ alkoxy, oxetanyl, and tetrahyropyranyl, and wherein the remainder of the variables are as defined in the fifth embodiment.

In a seventh embodiment, the present disclosure provides a compound of Formula (I), (II), (III), or (III-A), or a pharmaceutically acceptable salt thereof, wherein R¹ is: C₁-C₄ alkyl optionally substituted with 1 or 2 groups selected from N(CH₃)₂, ═O, azetidinyl, oxetanyl, and morpholinyl; oxetanyl; pyrrolidinyl optionally substituted with 1 or 2 groups selected from methyl and ═O; or piperidinyl optionally substituted with F or oxetanyl, and wherein the remainder of the variables are as defined in the sixth embodiment.

In an eighth embodiment, the present disclosure provides a compound of Formula (I), (II), (III), or (III-A), or a pharmaceutically acceptable salt thereof, wherein R² is C₁-C₄ alkyl, C₂-C₄ alkenyl, C₂-C₄ alkynyl, C₁-C₄ alkoxy, 4- to 10-membered heterocyclyl, phenyl, or 5- or 6-membered heteroaryl, wherein the alkyl, alkenyl, alkynyl, and alkoxy represented by R² are each optionally substituted with 1 to 4 groups selected from deuterium, halo, ═O (as valence permits), OH, NR^{1a}R^{1b}, C(O)R^{1c}, C(O)OR^{1c}, —S(O)₂R^{1c}, C₁-C₄ alkyl, C₁-C₄ alkoxy, and 4- to 6-membered heterocyclyl (optionally substituted with ═O, NR^{1a}R^{1b}, C₁-C₄ alkyl); wherein the 4- to 10-membered heterocycyl, phenyl, and 5- or 6-heteroaryl represented by R² are each optionally substituted with 1 to 4 groups selected from deuterium, halo, ═O (as valence permits), OH, NR^{1a}R^{1b}, C(O)R^{1c}, C(O)OR^{1c}, —S(O)₂R^{1c}, C₁-C₄ alkyl (optionally substituted with 1 to 4 groups selected from halo, —OH and C₁-C₄ alkoxy), C₁-C₄ alkoxy (optionally substituted with 1 to 4 groups selected from halo and —OH), and 4- to 6-membered heterocyclyl (optionally substituted with 1 to 2 groups selected from ═O, NR^{1a}R^{1b}, C₁-C₄ alkyl), and wherein the remainder of the variables are as defined in the first, second, third, fourth, fifth, or sixth embodiment.

In a ninth embodiment, the present disclosure provides a compound of Formula (I), (II), (III), or (III-A), or a pharmaceutically acceptable salt thereof, wherein R² is: C₁-C₄ alkoxy optionally substituted with 1 to 4 groups selected from deuterium, halo, ═O (as valence permits), C₁-C₄ alkoxy, and 4- to 6-membered heterocyclyl (optionally substituted with ═O, NR^{1a}R^{1b}, C₁-C₄ alkyl); C₂-C₄ alkynyl optionally substituted with 1 to 2 groups selected from C₁-C₄ alkyl and 4- to 6-membered heterocyclyl (optionally substituted with ═O, NR^{1a}R^{1b}, C₁-C₄ alkyl); 4- to 10-membered heterocyclyl, phenyl, or 5- or 6-membered heteroaryl each optionally substituted with 1 to 4 groups selected from halo, ═O (as valence permits), C₁-C₄ alkyl (optionally substituted with 1 to 4 groups selected from halo, —OH and C₁-C₄ alkoxy), C₁-C₄ alkoxy (optionally substituted with 1 to 4 groups selected from halo and —OH), —C(O)OC₁-C₄ alkyl, —S(O)₂C1-C₄ alkyl, and 4- to 6-membered heterocyclyl (optionally substituted with 1 to 2 groups selected from ═O, NR^{1a}R^{1b}, C₁-C₄ alkyl), and wherein the remainder of the variables are as defined in the eighth embodiment.

In a tenth embodiment, the present disclosure provides a compound of Formula (I), (II), (III), or (III-A), or a pharmaceutically acceptable salt thereof, wherein R² is: C₁-C₄ alkoxy optionally substituted with 1 to 3 groups selected from deuterium, OCH₃, and piperizinyl (optionally substituted with methyl); C₂-C₄ alkynyl optionally substituted with 4- to 6-membered heterocyclyl (optionally substituted with C₁-C₂alkyl); 1,3-dihydro-2H-imidazol-2-onyl optionally substituted with 1 or 2 C₁-C₂ alkyl; 2,5 [symbol] -diazabicyclo[4.1.0]heptanyl optionally substituted with C₁-C₂ alkyl; 6-oxa-3-azabicyclo[3.1.1]heptanyl; 2-oxa-6 [symbol] -azaspiro[3.3]heptanyl; dihydro-3H-pyrazol-3-onyl optionally substituted with 1 or 2 groups selected from C₁-C₃ alkyl (optionally substituted with 1 to 3 halo); hexahydro-1H-2 [symbol] -pyrrolo[2,1-c]pyrazinyl; imidazolidinyl optionally substituted with 1 or 2 groups selected from ═O and C₁-C₃ alkyl; morpholinyl optionally substituted with 1 or 2 groups selected from C₁-C₃ alkyl; phenyl optionally substituted with S(O)₂CH₃; piperizinyl optionally substituted with 1 or 3 groups selected from C₁-C₃ alkyl (optionally substituted with —OH or C₁-C₂ alkoxy) and —C(O)OC₁-C₄ alkyl; pyrazolyl optionally substituted with 1 or 2 groups selected from piperizinyl (optional substituted with methyl), piperidinyl (optionally substituted with methyl), C₁-C₄ alkyl, and C₁-C₄ alkoxy, wherein the C₁-C₄ alkyl and C₁-C₄ alkoxy are each optionally substituted with 1 to 4 groups selected from halo and OH; pyrrolidinyl optionally substituted with 1 or 2 groups selected from C₁-C₃ alkyl (optionally substituted with —OH) and C₁-C₂alkoxy; or triazolyl optionally substituted with C₁-C₃ alkyl, and wherein the remainder of the variables are as defined in the nineth embodiment.

In an eleventh embodiment, the present disclosure provides a compound of Formula (I), (II), (III), or (III-A), or a pharmaceutically acceptable salt thereof, wherein R² is: pyrazolyl optional substituted with 1 or 2 groups selected from methyl, methoxy, —OCHF₂, —CH₂C(OH)(CH₃)₂, —CH(CH₃)CH₂OH, and —C(CH₃)₂CH₂OH; pyrrolidinyl optionally substituted with 1 or 2 groups selected from methoxy and —C(OH)(CH₃)₂; 6-oxa-3-azabicyclo[3.1.1]heptanyl; or 2-oxa-6 [symbol] -azaspiro[3.3]heptanyl, and wherein the remainder of the variables are as defined in the tenth embodiment.

In a twelfth embodiment, the compound of Formula (I) or (II) is represented by Formula (IV):

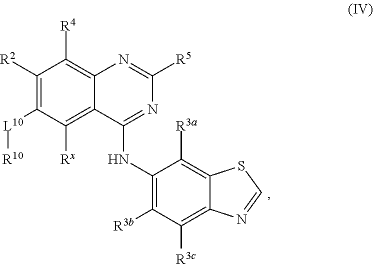

or a pharmaceutically acceptable salt thereof, wherein R^{x} is H, F; R^{3a} is H, deuterium, or halo; R^{3b} is H, deuterium, or halo; and R^{3c} is H, deuterium, or halo, and wherein the remainder of the variables are as defined in the first or second embodiment.

In a thirteenth embodiment, the present disclosure provides a compound of Formula (I), (II) or (IV), or a pharmaceutically acceptable salt thereof, wherein L¹⁰ is a bond, NH, —NHC(O)—*, or O; wherein —* represents the point which attaches to R¹⁰, and wherein the remainder of the variables are as defined in the twelfth embodiment.

In a fourteenth embodiment, the present disclosure provides a compound of Formula (I), (II) or (IV), or a pharmaceutically acceptable salt thereof, wherein L¹⁰⁻R¹⁰ is H or halo; or R¹⁰ is: C₁-C₄ alkyl optionally substituted with 1 to 4 groups independently selected from halo, OR^{1a}, NR^{1a}R^{1b}, 4 to 6 membered heterocyclyl wherein the 4 to 6 membered heterocyclyl is optionally substituted with 1 to 2 groups independently selected from halo, ═O, NR^{1a}R^{1b}, and C₁-C₄ alkyl; or 4- to 8-membered heterocyclyl optionally substituted with 1 to 2 groups selected from halo, ═O, C₁-C₄ alkyl, OR^{1a}, C(O)R^{1a}; C(O)NR^{1a}R^{1b}, NR^{1a}C(O)OR^{1a}, and NR^{1a}R^{1b}; or 5- to 6-membered heteroaryl optionally substituted with 1 to 2 groups selected from halo, C₁-C₄ alkyl, OR^{1a}, C(O)R^{1a}; C(O)NR^{1a}R^{1b}, NR^{1a}C(O)OR^{1a}, NR^{1a}R^{1b}, and 4- to 6-membered heterocyclyl (which is further optionally substituted with 1 or 2 groups selected from halo, ═O, NR^{1a}R^{1b}, and C₁-C₄ alkyl), and wherein the remainder of the variables are as defined in the thirteenth embodiment.

In a fifteenth embodiment, the present disclosure provides a compound of Formula (I), (II) or (IV), or a pharmaceutically acceptable salt thereof, wherein L¹⁰-R¹⁰ is H or halo, and wherein the remainder of the variables are as defined in the fourteenth embodiment.

In a sixteenth embodiment, the present disclosure provides a compound of Formula (I), (II) or (IV), or a pharmaceutically acceptable salt thereof, wherein L¹⁰ is a bond; and R¹⁰ is 5- to 6-membered heteroaryl optionally substituted with C₁-C₂ alkyl or 4- to 6-membered heterocyclyl (which is further optionally substituted with 1 or 2 groups selected from halo and C₁-C₂ alkyl) (e.g., pyrazolyl optionally substituted with methyl or piperidinyl (optionally substituted with 1 or 2 groups selected from F and methyl), and wherein the remainder of the variables are as defined in the fourteenth embodiment.

In a seventeenth embodiment, the present disclosure provides a compound of Formula (I), (II) or (IV), or a pharmaceutically acceptable salt thereof, wherein L¹⁰ is NH; and R¹⁰ is 4- to 6-membered heterocyclyl optionally substituted with 1 to 2 groups selected from halo, C₁-C₂ alkyl, and —C(O)C₁-C₂ alkyl (e.g., piperidinyl optionally substituted with 1 or 2 groups selected from F, methyl, and —C(O)CH₂CH₃), and wherein the remainder of the variables are as defined in the fourteenth embodiment.

In a eighteenth embodiment, the present disclosure provides a compound of Formula (I), (II) or (IV), or a pharmaceutically acceptable salt thereof, wherein L¹⁰ is —NHC(O)—*, wherein —* represents the point which attaches to R¹⁰, and R¹⁰ is: C₁-C₂ alkyl optionally substituted with 4 to 6 membered heterocyclyl (e.g., ethyl optionally substituted with piperidinyl); or 4- to 8-membered heterocyclyl optionally substituted with 1 to 2 groups selected from halo, C₁-C₂ alkyl, and —C(O)C₁-C₂ alkyl (e.g., azetidinyl optionally substituted with methyl, piperidinyl optionally substituted with 1 or 2 groups selected from methyl and fluoro, piperizinyl optionally substituted with 1 or 2 groups selected from methyl and ethyl, octahydropyrrolo[3,4-b]pyrrolyl optionally substituted with methyl), and wherein the remainder of the variables are as defined in the fourteenth embodiment.

In a nineteenth embodiment, the present disclosure provides a compound of Formula (I), (II) or (IV), or a pharmaceutically acceptable salt thereof, wherein L¹⁰ is O; and R¹⁰ is: C₁-C₂ alkyl (e.g., CH₃); or 4- to 6-membered heterocyclyl optionally substituted with 1 to 2 groups selected from halo, C₁-C₂ alkyl, and —C(O)C₁-C₂ alkyl (e.g., tetrohydrofuranyl, tetrohydropyranyl, or piperidinyl optionally substituted with —C(O)CH₂CH₃), and wherein the remainder of the variables are as defined in the fourteenth embodiment.

In a twentieth embodiment, the present disclosure provides a compound of Formula (I), (II) or (IV), or a pharmaceutically acceptable salt thereof, wherein R² is C₁-C₄ alkyl or 5- or 6-membered heteroaryl (e.g., 5-membered heteroaryl), wherein the heteroaryl represented by R² is optionally substituted with 1 to 4 groups selected from C₁-C₄ alkyl, C₁-C₄ alkoxy, and 4 to 6 membered heterocyclyl (optionally substituted with C₁-C₄ alkyl), and wherein the remainder of the variables are as defined in the twelfth, thirteenth, fourteenth, fifteenth, sixteenth, seventeenth, eighteenth, or nineteenth embodiment.

In a twenty-first embodiment, the present disclosure provides a compound of Formula (I), (II) or (IV), or a pharmaceutically acceptable salt thereof, wherein R² is methyl or pyrazolyl optionally substituted with 1 or 2 groups selected from methyl, methoxy, and piperidinyl (optionally substituted with methyl), and wherein the remainder of the variables are as defined in the twentieth embodiment.

In a twenty-second embodiment, the present disclosure provides a compound of Formula (I), (II) or (IV), or a pharmaceutically acceptable salt thereof, wherein R^{x} is H, and wherein the remainder of the variables are as defined in the twelfth, thirteenth, fourteenth, fifteenth, sixteenth, seventeenth, eighteenth, nineteenth, twentieth or twenty-first embodiment.

In a twenty-third embodiment, the present disclosure provides a compound of Formula (I), (II), (III), (III-A) or (IV), or a pharmaceutically acceptable salt thereof, wherein R^{3a} is H or halo; R^{3b} and R^{3c} are H; and R⁴ and R⁵ are H, and wherein the remainder of the variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, or twenty-second embodiment.

In a twenty-fourth embodiment, the present disclosure provides a compound of Formula (I), (II), (III), (III-A) or (IV), or a pharmaceutically acceptable salt thereof, wherein R^{5a} is H or F, and wherein the remainder of the variables are as defined in the twenty-third embodiment.

In a twenty-fifth embodiment, the present disclosure provides a compound of Formula (I), (II), (III), (III-A) or (IV), or a pharmaceutically acceptable salt thereof, wherein R^{1a} is H or C₁-C₄ alkyl; R^{1b} is H or C₁-C₄ alkyl; and R^{1c} is H or C₁-C₄ alkyl optionally substituted with 1 to 3 halo (F), and wherein the remainder of the variables are as defined in the first, second, third, fourth, fifth, sixth, seventh, eighth, ninth, tenth, eleventh, twelfth, thirteenth, fourteenth, fifteenth, sixteenth, seventeenth, eighteenth, nineteenth, twentieth, twenty-first, twenty-second, twenty-third or twenty-fourth embodiment.

In a twenty-sixth embodiment, a compound of the present disclosure is any one of the compounds disclosed in the examples (including neutral form, pharmaceutically acceptable salts, and intermediates) and Table 1, or a pharmaceutically acceptable salt thereof.

TABLE 1

| Example # | Structure |
|---|---|
| 1 | 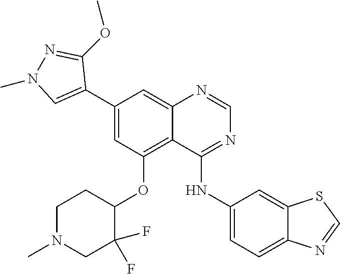 [SMILES: COc1nn(C)cc1-c1cc(OC2CCN(C)CC2(F)F)c2c(Nc3ccc4ncsc4c3)ncnc2c1] |
| 2 | 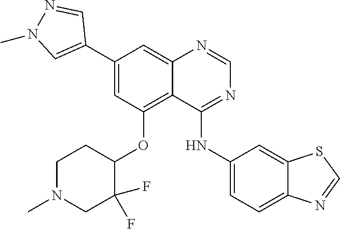 [SMILES: CN1CCC(Oc2cc(-c3cnn(C)c3)cc3ncnc(Nc4ccc5ncsc5c4)c23)C(F)(F)C1] |
| 3 | 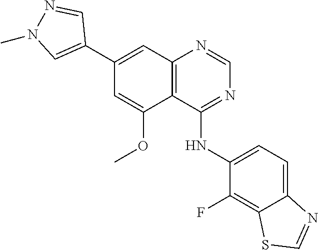 [SMILES: COc1cc(-c2cnn(C)c2)cc2ncnc(Nc3ccc4ncsc4c3F)c12] |
| 4 | 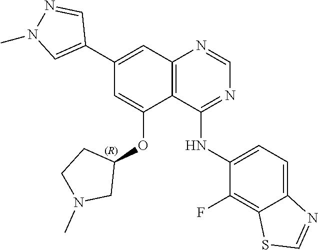 |
| 5 | 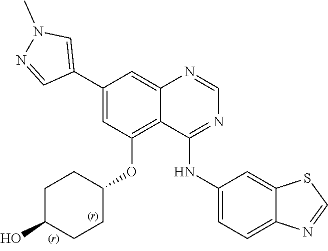 [SMILES: Cn1cc(-c2cc(O[C@H]3CC[C@H](O)CC3)c3c(Nc4ccc5ncsc5c4)ncnc3c2)cn1] |
| 6 and 7 | 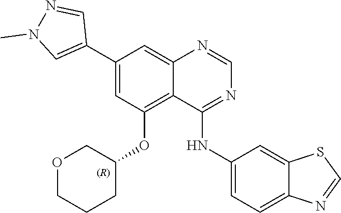 |
| | or |
| | 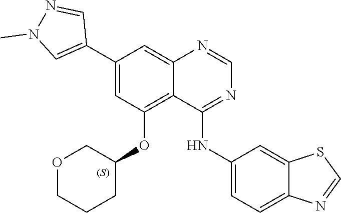 |
| 8 | 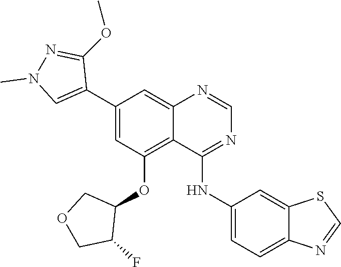 [SMILES: COc1nn(C)cc1-c1cc(O[C@@H]2COC[C@H]2F)c2c(Nc3ccc4ncsc4c3)ncnc2c1] |
| | trans-racemic |
| 9 | 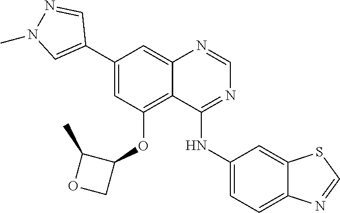 [SMILES: C[C@@H]1OC[C@@H]1Oc1cc(-c2cnn(C)c2)cc2ncnc(Nc3ccc4ncsc4c3)c12] |
| | cis-racemic |
| 10 | 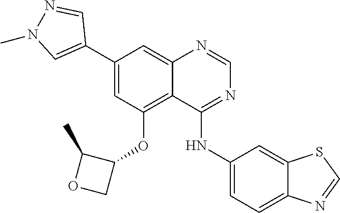 [SMILES: C[C@@H]1OC[C@H]1Oc1cc(-c2cnn(C)c2)cc2ncnc(Nc3ccc4ncsc4c3)c12] |
| | trans-racemic |
| 11 | 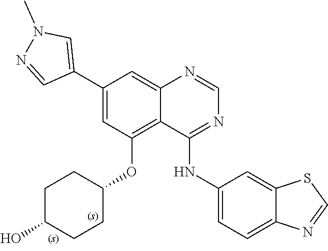 [SMILES: Cn1cc(-c2cc(O[C@H]3CC[C@@H](O)CC3)c3c(Nc4ccc5ncsc5c4)ncnc3c2)cn1] |
| 12 | 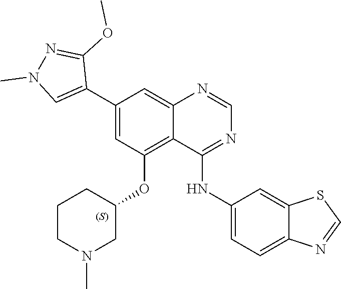 |
| 13 | 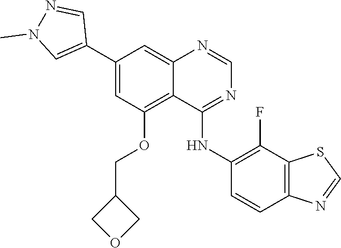 [SMILES: Cn1cc(-c2cc(OCC3COC3)c3c(Nc4ccc5ncsc5c4F)ncnc3c2)cn1] |
| 14 | 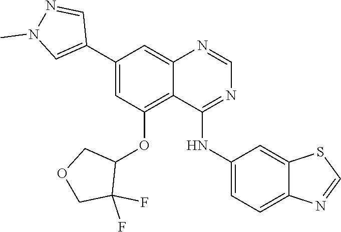 [SMILES: Cn1cc(-c2cc(OC3COCC3(F)F)c3c(Nc4ccc5ncsc5c4)ncnc3c2)cn1] |
| 15 | 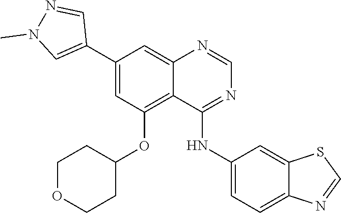 [SMILES: Cn1cc(-c2cc(OC3CCOCC3)c3c(Nc4ccc5ncsc5c4)ncnc3c2)cn1] |
| 16 | 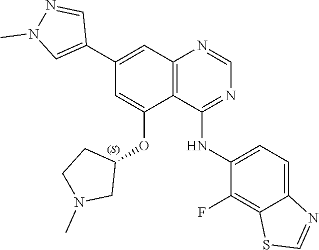 |
| 17 | 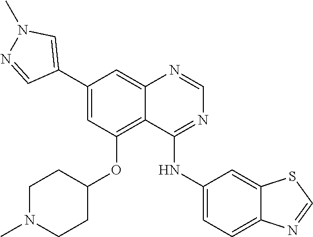 [SMILES: CN1CCC(Oc2cc(-c3cnn(C)c3)cc3ncnc(Nc4ccc5ncsc5c4)c23)CC1] |
| 18 | 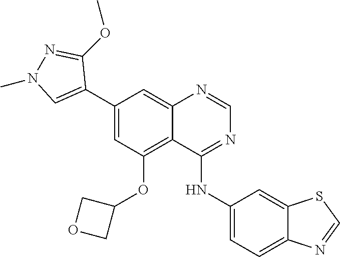 [SMILES: COc1nn(C)cc1-c1cc(OC2COC2)c2c(Nc3ccc4ncsc4c3)ncnc2c1] |
| 19 | 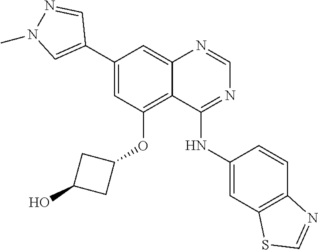 [SMILES: Cn1cc(-c2cc(O[C@H]3C[C@H](O)C3)c3c(Nc4ccc5ncsc5c4)ncnc3c2)cn1] |
| 20 | 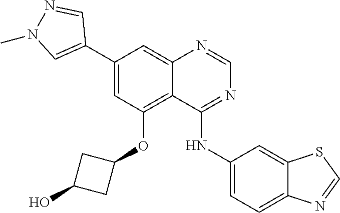 [SMILES: Cn1cc(-c2cc(O[C@H]3C[C@@H](O)C3)c3c(Nc4ccc5ncsc5c4)ncnc3c2)cn1] |
| 21 | 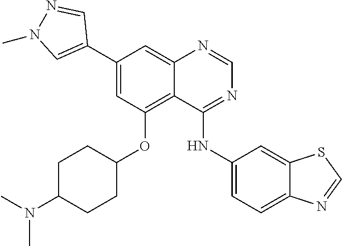 [SMILES: CN(C)C1CCC(Oc2cc(-c3cnn(C)c3)cc3ncnc(Nc4ccc5ncsc5c4)c23)CC1] |
| 22 | 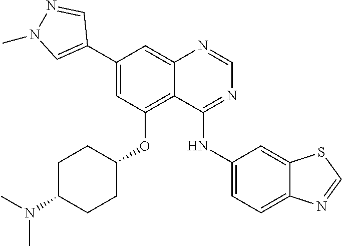 [SMILES: Cn1cc(-c2cc(O[C@H]3CC[C@@H](N(C)C)CC3)c3c(Nc4ccc5ncsc5c4)ncnc3c2)cn1] |
| 23 and 24 | 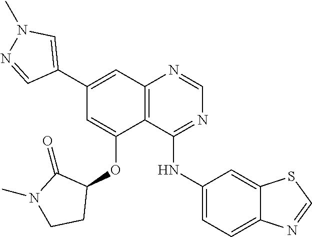 [SMILES: CN1CC[C@H](Oc2cc(-c3cnn(C)c3)cc3ncnc(Nc4ccc5ncsc5c4)c23)C1=O] |
| | OR |
| | 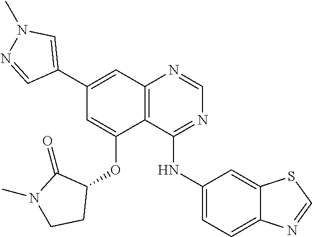 [SMILES: CN1CC[C@@H](Oc2cc(-c3cnn(C)c3)cc3ncnc(Nc4ccc5ncsc5c4)c23)C1=O] |
| 25 | 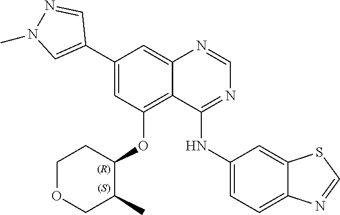 |
| | cis-racemic |
| 26 | 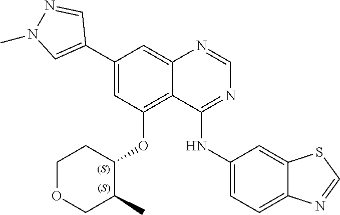 |
| | trans-racemic |
| 27 | 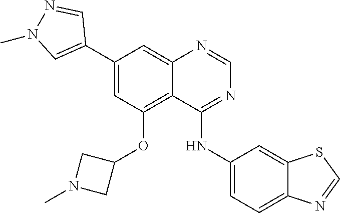 [SMILES: CN1CC(Oc2cc(-c3cnn(C)c3)cc3ncnc(Nc4ccc5ncsc5c4)c23)C1] |
| 28 | 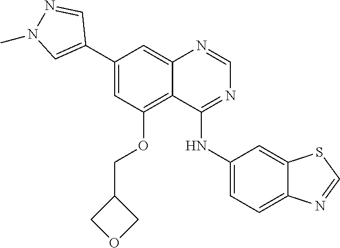 [SMILES: Cn1cc(-c2cc(OCC3COC3)c3c(Nc4ccc5ncsc5c4)ncnc3c2)cn1] |
| 29 | 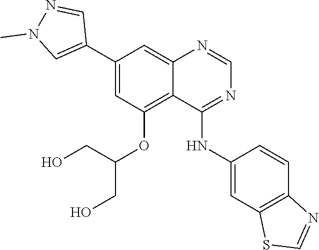 [SMILES: Cn1cc(-c2cc(OC(CO)CO)c3c(Nc4ccc5ncsc5c4)ncnc3c2)cn1] |
| 30 | 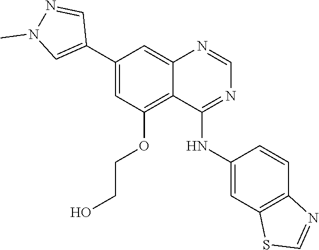 [SMILES: Cn1cc(-c2cc(OCCO)c3c(Nc4ccc5ncsc5c4)ncnc3c2)cn1] |
| 31 | 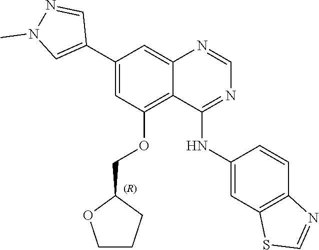 |
| 32 | 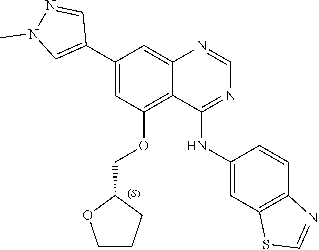 |
| 33 | 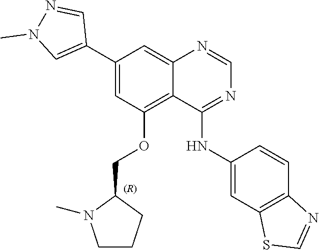 |
| 34 | 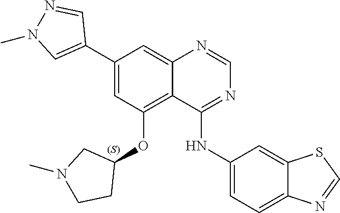 |
| 35 | 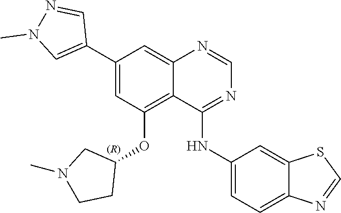 |
| 36 | 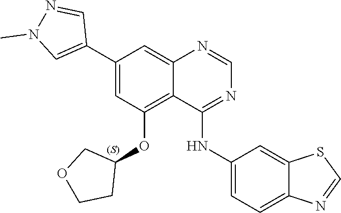 |
| 37 | 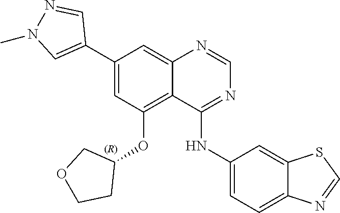 |
| 38 | 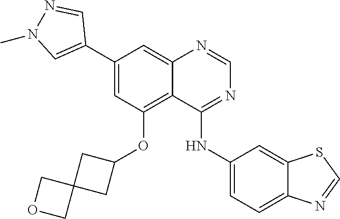 [SMILES: Cn1cc(-c2cc(OC3CC4(COC4)C3)c3c(Nc4ccc5ncsc5c4)ncnc3c2)cn1] |
| 41 and 42 | 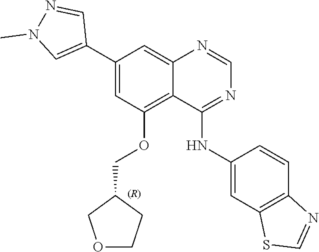 |
| | or |
| | 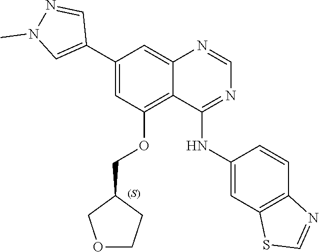 |
| 43 and 44 | 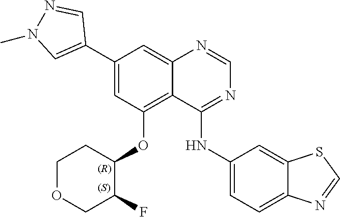 |
| | or |
| | 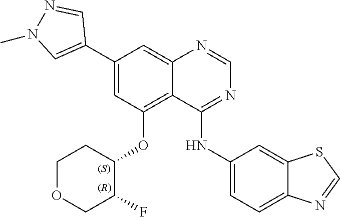 |
| 45 and 46 | 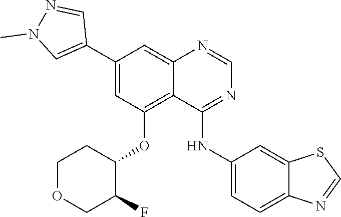 [SMILES: Cn1cc(-c2cc(O[C@H]3CCOC[C@@H]3F)c3c(Nc4ccc5ncsc5c4)ncnc3c2)cn1] |
| | or |
| | 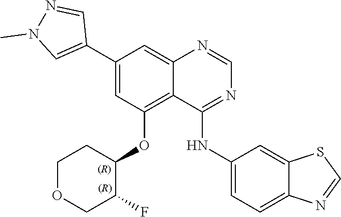 |
| 47 and 48 | 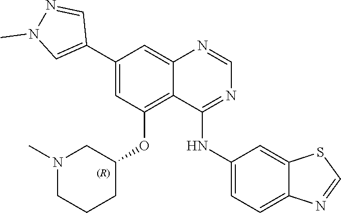 |
| | or |
| | 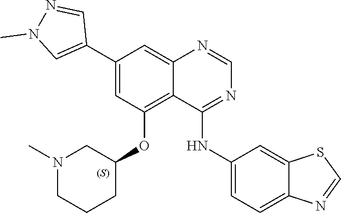 |
| 49 | 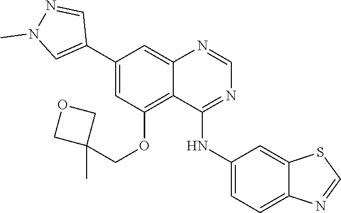 [SMILES: Cn1cc(-c2cc(OCC3(C)COC3)c3c(Nc4ccc5ncsc5c4)ncnc3c2)cn1] |
| 50 | 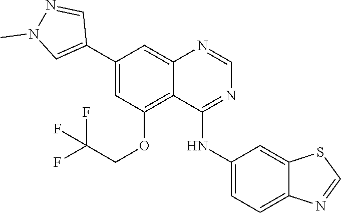 [SMILES: Cn1cc(-c2cc(OCC(F)(F)F)c3c(Nc4ccc5ncsc5c4)ncnc3c2)cn1] |
| 51 | 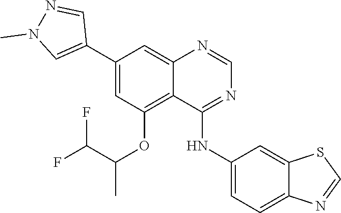 [SMILES: CC(Oc1cc(-c2cnn(C)c2)cc2ncnc(Nc3ccc4ncsc4c3)c12)C(F)F] |
| 52 and 53 | 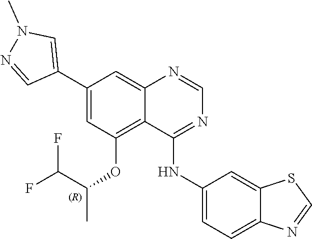 |
| | or |
| | 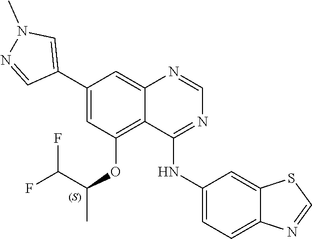 |
| 54 | 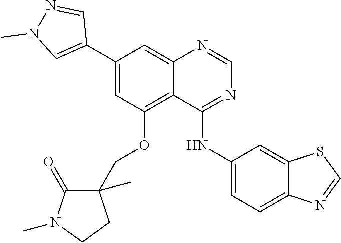 [SMILES: CN1CCC(C)(COc2cc(-c3cnn(C)c3)cc3ncnc(Nc4ccc5ncsc5c4)c23)C1=O] |
| 55 and 56 | 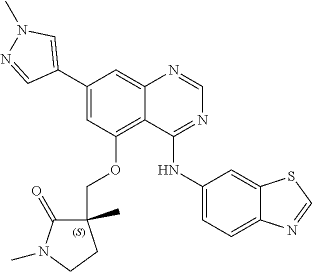 |
| | or |
| | 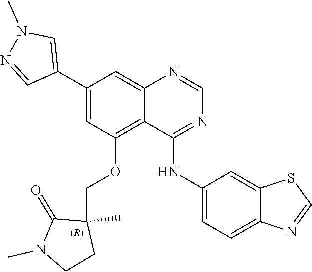 |
| 57 | 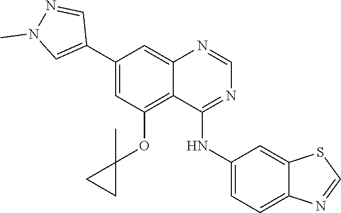 [SMILES: Cn1cc(-c2cc(OC3(C)CC3)c3c(Nc4ccc5ncsc5c4)ncnc3c2)cn1] |
| 58 | 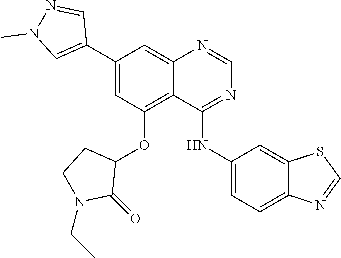 [SMILES: CCN1CCC(Oc2cc(-c3cnn(C)c3)cc3ncnc(Nc4ccc5ncsc5c4)c23)C1=O] |
| 59 and 60 | 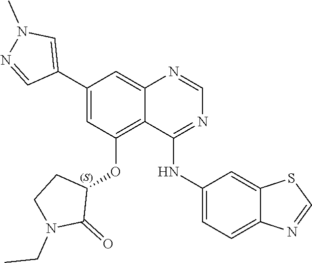 |
| | or |
| | 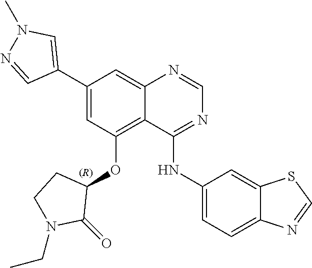 |
| 61 | 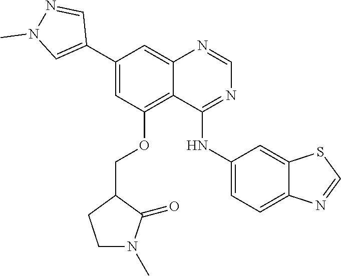 [SMILES: CN1CCC(COc2cc(-c3cnn(C)c3)cc3ncnc(Nc4ccc5ncsc5c4)c23)C1=O] |
| 62 | 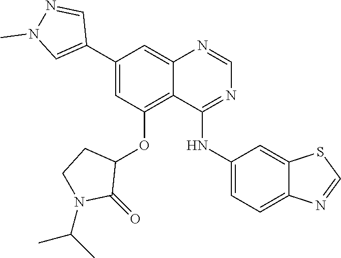 [SMILES: CC(C)N1CCC(Oc2cc(-c3cnn(C)c3)cc3ncnc(Nc4ccc5ncsc5c4)c23)C1=O] |
| 63 and 64 | 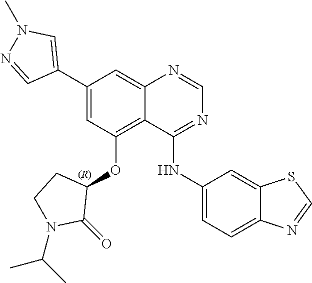 |
| | or |
| | 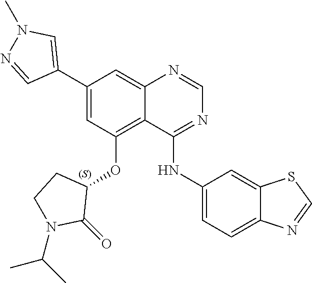 |
| 65 | 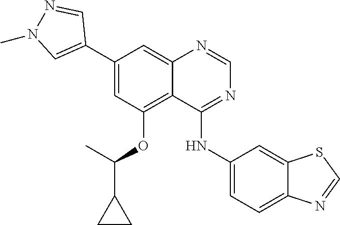 [SMILES: C[C@@H](Oc1cc(-c2cnn(C)c2)cc2ncnc(Nc3ccc4ncsc4c3)c12)C1CC1] |
| 66 | 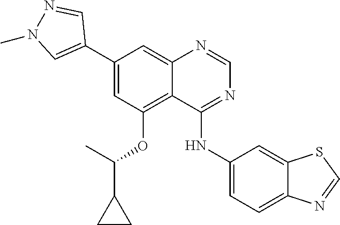 [SMILES: C[C@H](Oc1cc(-c2cnn(C)c2)cc2ncnc(Nc3ccc4ncsc4c3)c12)C1CC1] |
| 67 | 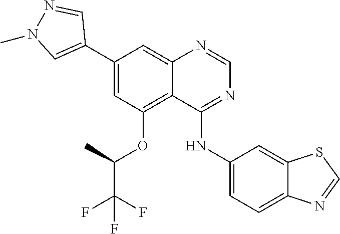 [SMILES: C[C@@H](Oc1cc(-c2cnn(C)c2)cc2ncnc(Nc3ccc4ncsc4c3)c12)C(F)(F)F] |
| 68 | 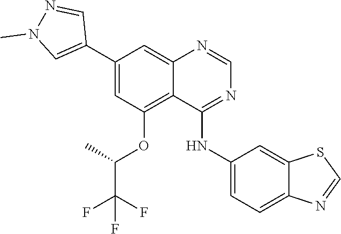 [SMILES: C[C@H](Oc1cc(-c2cnn(C)c2)cc2ncnc(Nc3ccc4ncsc4c3)c12)C(F)(F)F] |
| 69 | 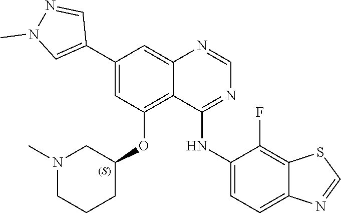 |
| 70 and 71 | 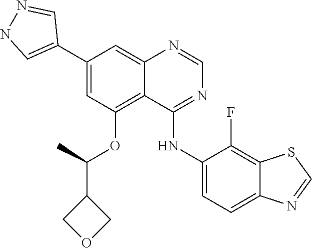 [SMILES: C[C@@H](Oc1cc(-c2cn[nH]c2)cc2ncnc(Nc3ccc4ncsc4c3F)c12)C1COC1] |
| | or |
| | 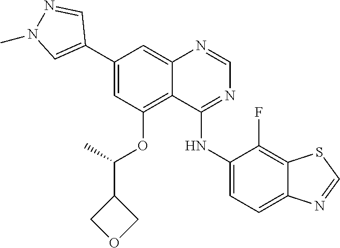 [SMILES: C[C@H](Oc1cc(-c2cnn(C)c2)cc2ncnc(Nc3ccc4ncsc4c3F)c12)C1COC1] |
| 72 | |
| 73 | 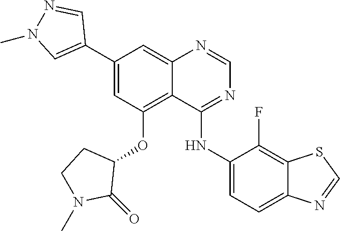 [SMILES: CN1CC[C@H](Oc2cc(-c3cnn(C)c3)cc3ncnc(Nc4ccc5ncsc5c4F)c23)C1=O] |
| | or |
| | 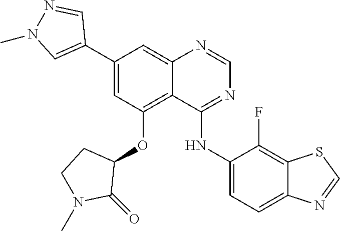 [SMILES: CN1CC[C@@H](Oc2cc(-c3cnn(C)c3)cc3ncnc(Nc4ccc5ncsc5c4F)c23)C1=O] |
| 74 and 75 | 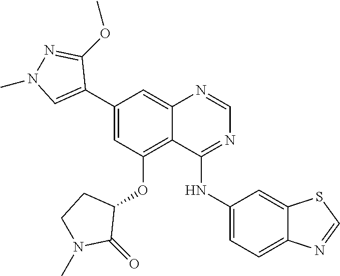 [SMILES: COc1nn(C)cc1-c1cc(O[C@H]2CCN(C)C2=O)c2c(Nc3ccc4ncsc4c3)ncnc2c1] |
| | or |
| | 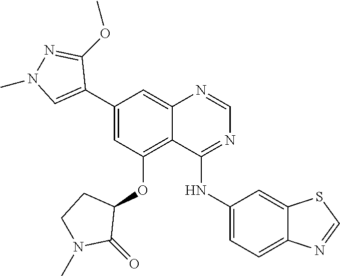 [SMILES: COc1nn(C)cc1-c1cc(O[C@@H]2CCN(C)C2=O)c2c(Nc3ccc4ncsc4c3)ncnc2c1] |
| 76 | 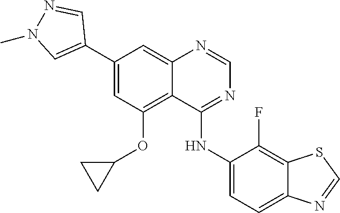 [SMILES: Cn1cc(-c2cc(OC3CC3)c3c(Nc4ccc5ncsc5c4F)ncnc3c2)cn1] |
| 77 | 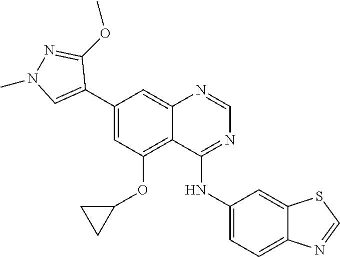 [SMILES: COc1nn(C)cc1-c1cc(OC2CC2)c2c(Nc3ccc4ncsc4c3)ncnc2c1] |
| 78 and 79 | 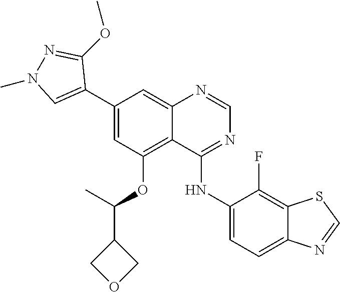 [SMILES: COc1nn(C)cc1-c1cc(O[C@H](C)C2COC2)c2c(Nc3ccc4ncsc4c3F)ncnc2c1] |
| | or |
| | 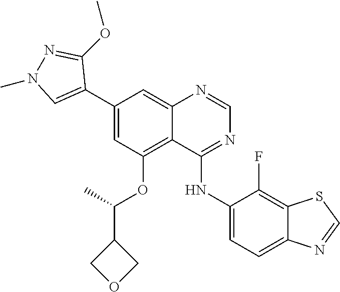 [SMILES: COc1nn(C)cc1-c1cc(O[C@@H](C)C2COC2)c2c(Nc3ccc4ncsc4c3F)ncnc2c1] |
| 80 and 81 | 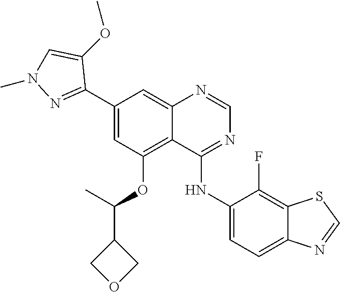 [SMILES: COc1cn(C)nc1-c1cc(O[C@H](C)C2COC2)c2c(Nc3ccc4ncsc4c3F)ncnc2c1] |
| | or |
| | 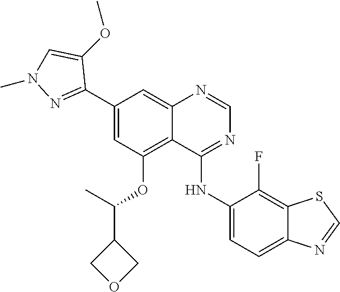 [SMILES: COc1cn(C)nc1-c1cc(O[C@@H](C)C2COC2)c2c(Nc3ccc4ncsc4c3F)ncnc2c1] |
| 82 and 83 | 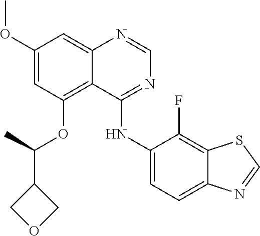 [SMILES: COc1cc(O[C@H](C)C2COC2)c2c(Nc3ccc4ncsc4c3F)ncnc2c1] |
| | or |
| | 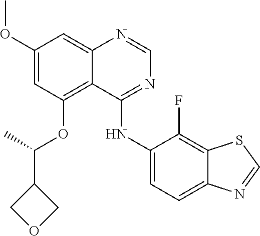 [SMILES: COc1cc(O[C@@H](C)C2COC2)c2c(Nc3ccc4ncsc4c3F)ncnc2c1] |
| 84 and 85 | 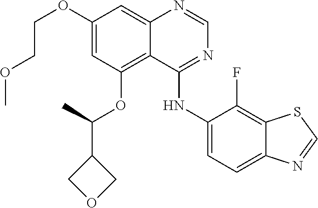 [SMILES: COCCOc1cc(O[C@H](C)C2COC2)c2c(Nc3ccc4ncsc4c3F)ncnc2c1] |
| | or |
| | 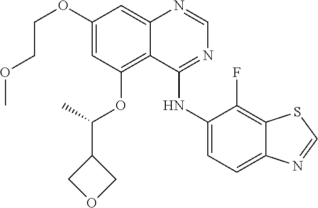 [SMILES: COCCOc1cc(O[C@@H](C)C2COC2)c2c(Nc3ccc4ncsc4c3F)ncnc2c1] |
| 86 and 87 | 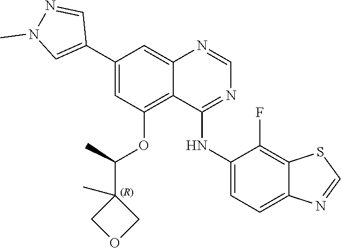 |
| | or |
| | 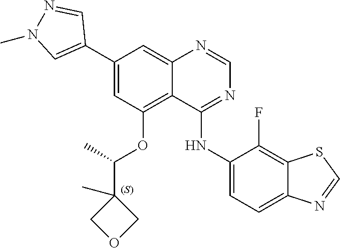 |
| 88 and 89 | 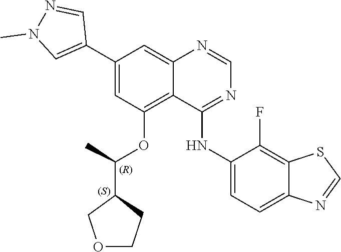 |
| | or |
| | 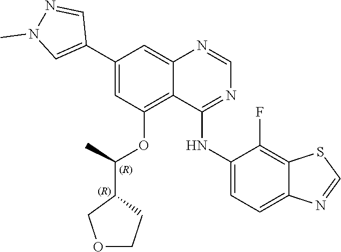 |
| 90 | 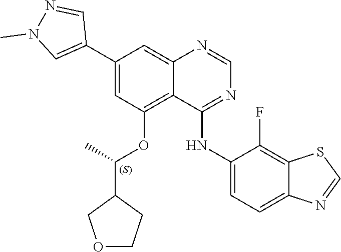 |
| 91 | 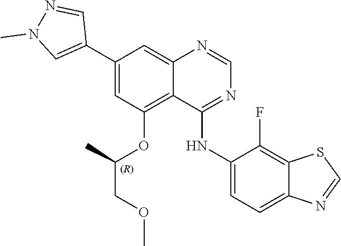 |
| 92 and 93 | 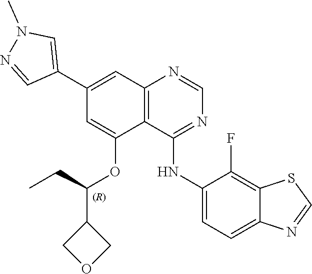 |
| | or |
| | 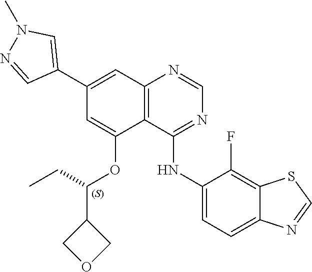 |
| 94 and 95 | 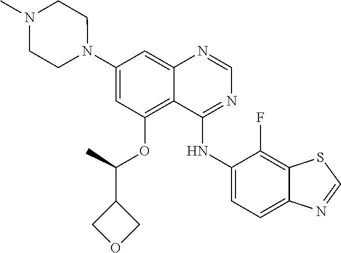 [SMILES: C[C@@H](Oc1cc(N2CCN(C)CC2)cc2ncnc(Nc3ccc4ncsc4c3F)c12)C1COC1] |
| | or |
| | 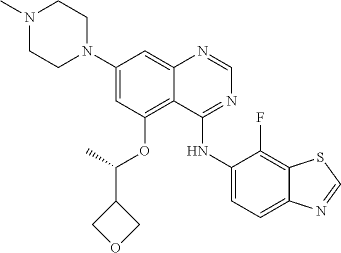 [SMILES: C[C@H](Oc1cc(N2CCN(C)CC2)cc2ncnc(Nc3ccc4ncsc4c3F)c12)C1COC1] |
| 96 and 97 | 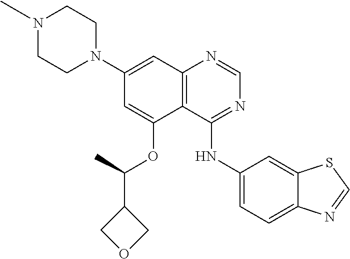 [SMILES: C[C@@H](Oc1cc(N2CCN(C)CC2)cc2ncnc(Nc3ccc4ncsc4c3)c12)C1COC1] |
| | or |
| | 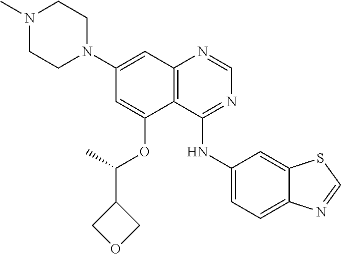 [SMILES: C[C@H](Oc1cc(N2CCN(C)CC2)cc2ncnc(Nc3ccc4ncsc4c3)c12)C1COC1] |
| 98 and 99 | 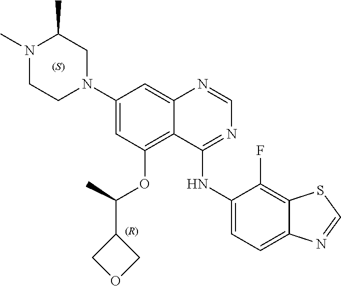 |
| | or |
| | 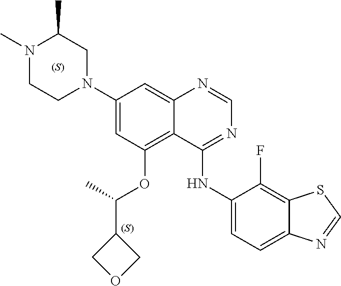 |
| 100 | 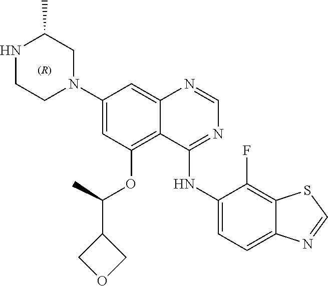 |
| | or |
| 101 | 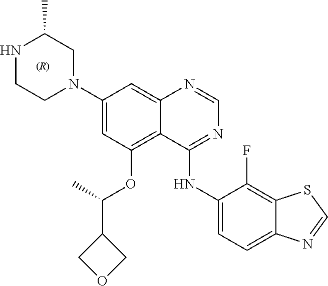 |
| 102 and 103 | 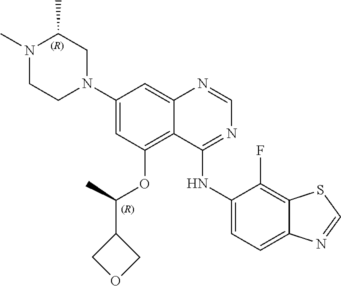 |
| | or |
| | 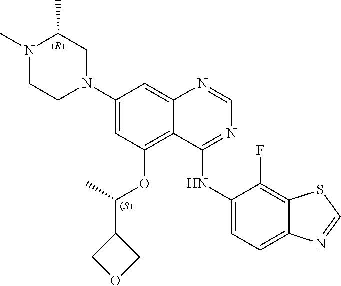 |
| 104 and 105 | 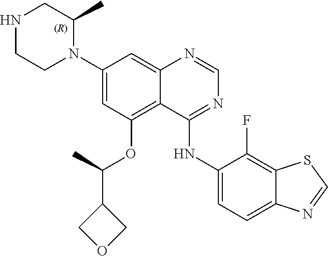 |
| | or |
| | 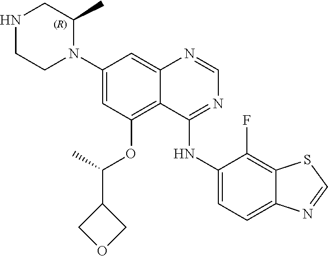 |
| 106 and 107 | 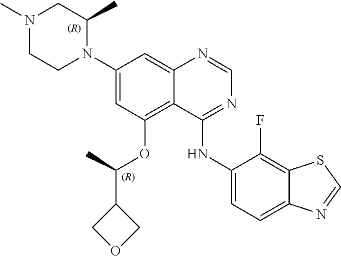 |
| | or |
| | 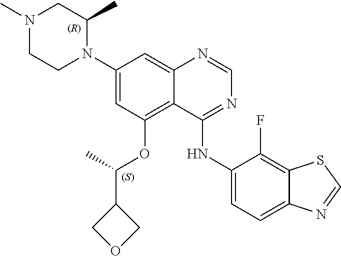 |
| 108 and 109 | 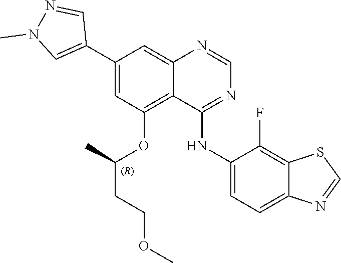 |
| | or |
| | 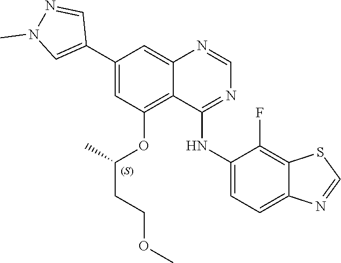 |
| 110 and 111 | 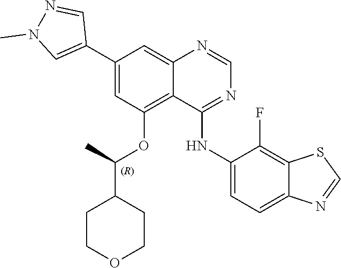 |
| | or |
| | 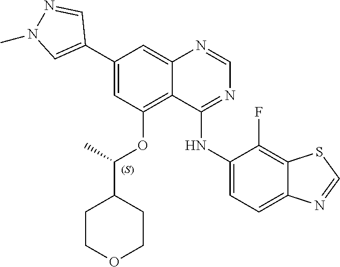 |
| 112 | 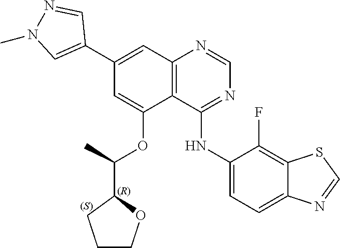 |
| 113 | 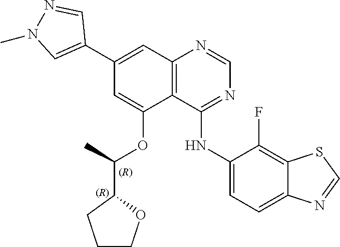 |
| 114 | 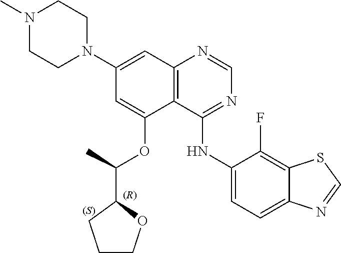 |
| 115 | 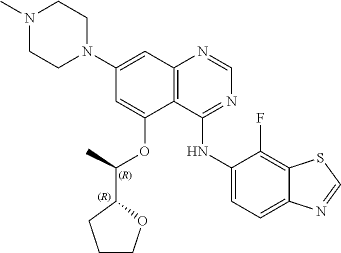 |
| 116 and 117 | 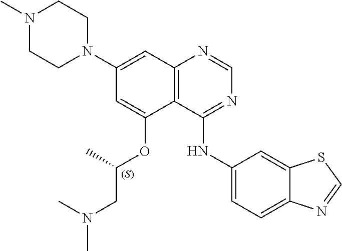 |
| | or |
| | 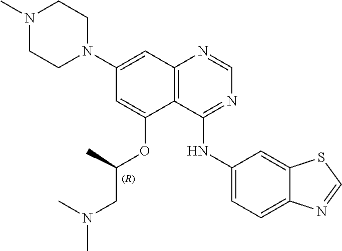 |
| 118 | 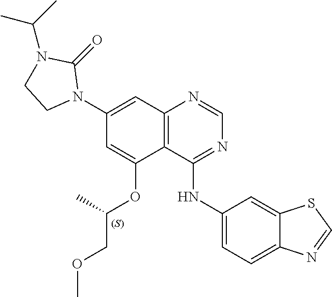 |
| | or |
| 119 | 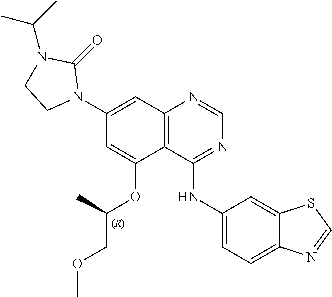 |
| 120 and 121 | 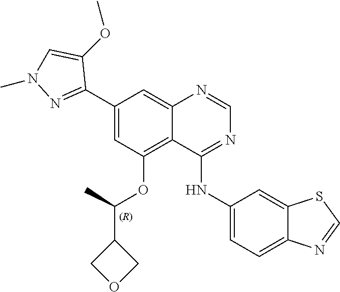 |
| | or |
| | 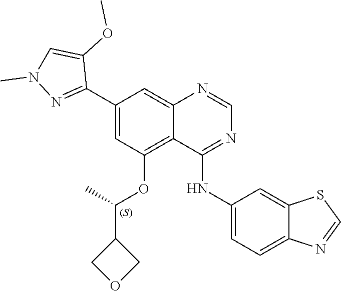 |
| 122 and 123 | 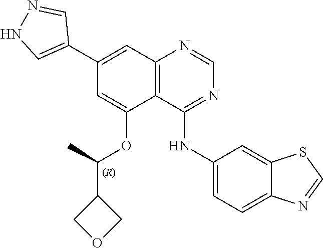 |
| | or |
| | 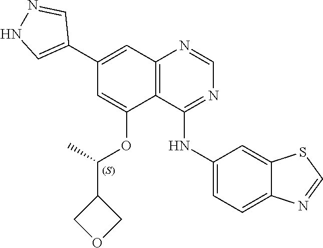 |
| 124 | 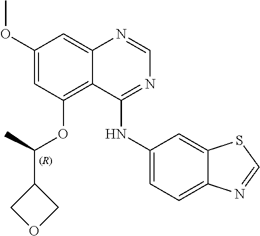 |
| 125 | 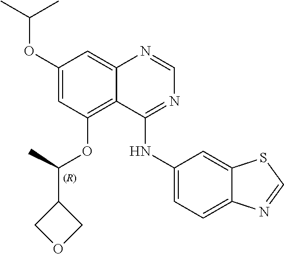 |
| 126 | 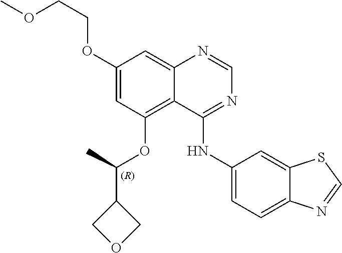 |
| 127. 128 | 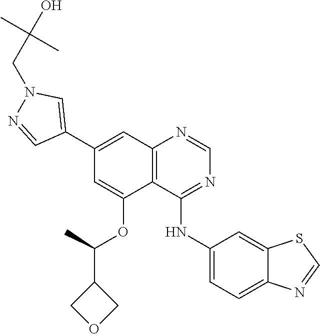 [SMILES: C[C@@H](Oc1cc(-c2cnn(CC(C)(C)O)c2)cc2ncnc(Nc3ccc4ncsc4c3)c12)C1COC1] |
| | or |
| | 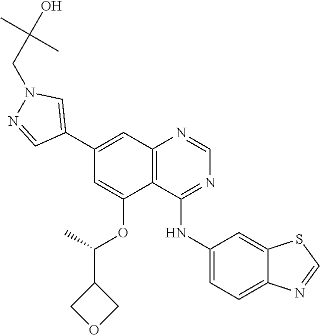 [SMILES: C[C@H](Oc1cc(-c2cnn(CC(C)(C)O)c2)cc2ncnc(Nc3ccc4ncsc4c3)c12)C1COC1] |
| 129 | 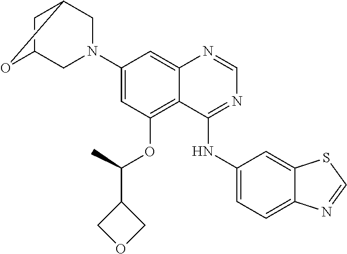 [SMILES: C[C@@H](Oc1cc(N2CC3CC(C2)O3)cc2ncnc(Nc3ccc4ncsc4c3)c12)C1COC1] |
| 130 | 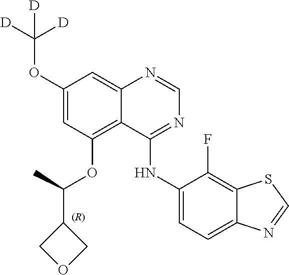 |
| 131 | 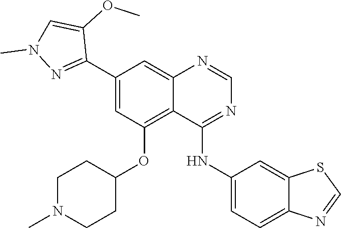 [SMILES: COc1cn(C)nc1-c1cc(OC2CCN(C)CC2)c2c(Nc3ccc4ncsc4c3)ncnc2c1] |
| 132 | 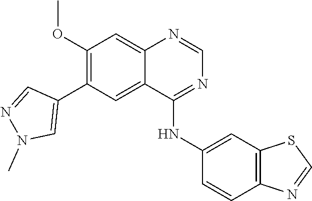 [SMILES: COc1cc2ncnc(Nc3ccc4ncsc4c3)c2cc1-c1cnn(C)c1] |
| 133 | 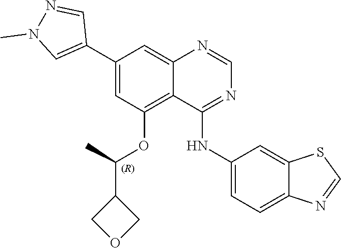 |
| 134 | 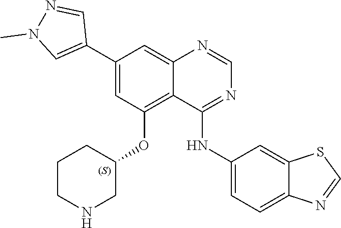 |
| 135 | 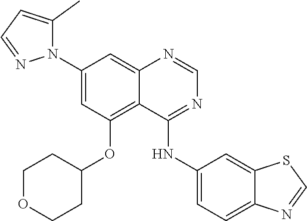 [SMILES: Cc1ccnn1-c1cc(OC2CCOCC2)c2c(Nc3ccc4ncsc4c3)ncnc2c1] |
| 136 | 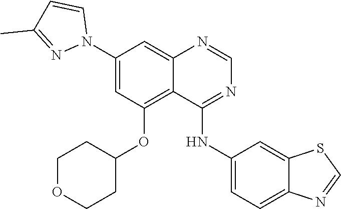 [SMILES: Cc1ccn(-c2cc(OC3CCOCC3)c3c(Nc4ccc5ncsc5c4)ncnc3c2)n1] |
| 139 | 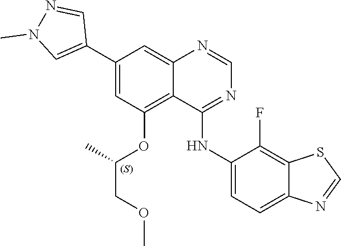 |
| 140 | 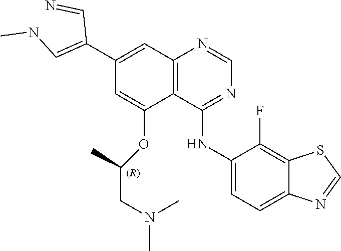 |
| 141 | 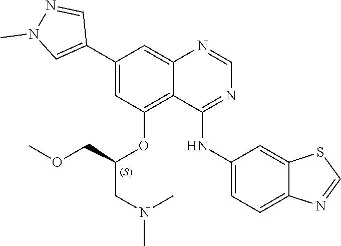 |
| 142 and 143 | 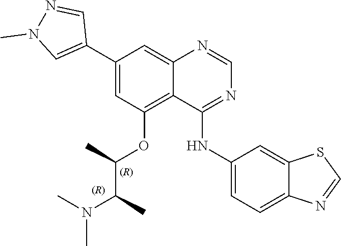 |
| | or |
| | 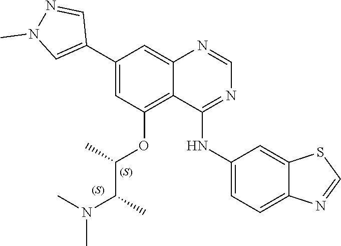 |
| 144 and 145 | 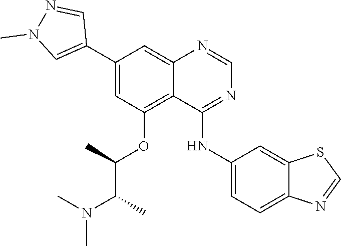 [SMILES: C[C@@H](Oc1cc(-c2cnn(C)c2)cc2ncnc(Nc3ccc4ncsc4c3)c12)[C@H](C)N(C)C] |
| | or |
| | 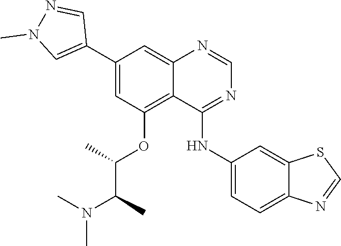 [SMILES: C[C@H](Oc1cc(-c2cnn(C)c2)cc2ncnc(Nc3ccc4ncsc4c3)c12)[C@@H](C)N(C)C] |
| 146 | 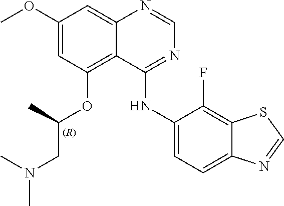 |
| 147 and 148 | 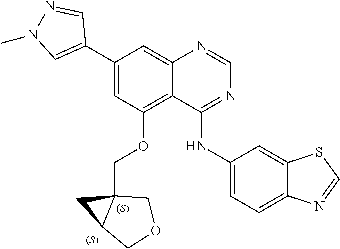 |
| | or |
| | 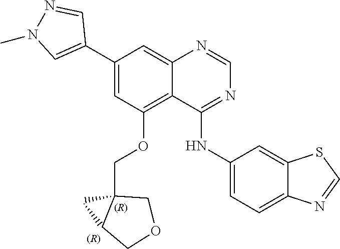 |
| 149 and150 | 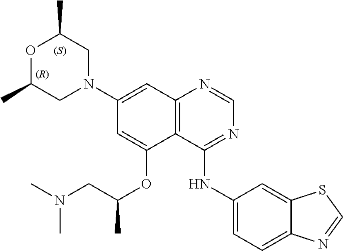 |
| | or |
| | 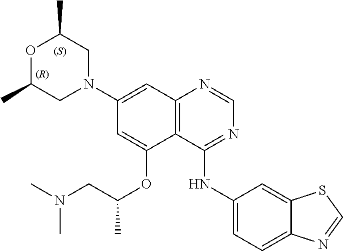 |
| | 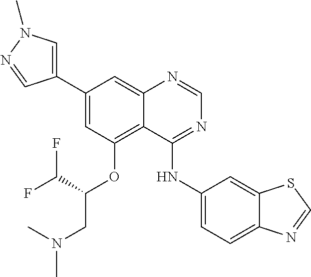 [SMILES: CN(C)C[C@@H](Oc1cc(-c2cnn(C)c2)cc2ncnc(Nc3ccc4ncsc4c3)c12)C(F)F] |
| | or |
| | 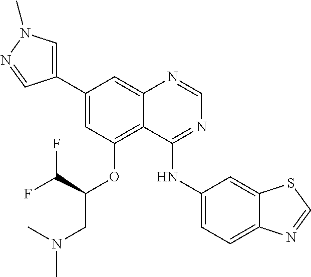 [SMILES: CN(C)C[C@H](Oc1cc(-c2cnn(C)c2)cc2ncnc(Nc3ccc4ncsc4c3)c12)C(F)F] |
| 153 and 154 | 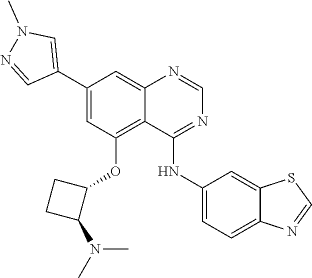 [SMILES: CN(C)[C@H]1CC[C@@H]1Oc1cc(-c2cnn(C)c2)cc2ncnc(Nc3ccc4ncsc4c3)c12] |
| | or |
| | 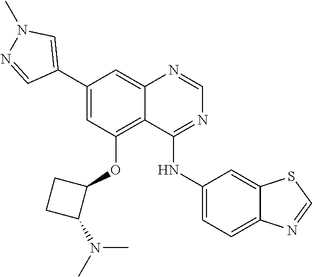 [SMILES: CN(C)[C@@H]1CC[C@H]1Oc1cc(-c2cnn(C)c2)cc2ncnc(Nc3ccc4ncsc4c3)c12] |
| 155 and 156 | 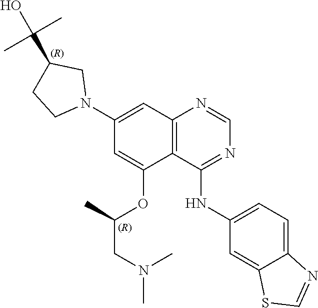 |
| | or |
| | 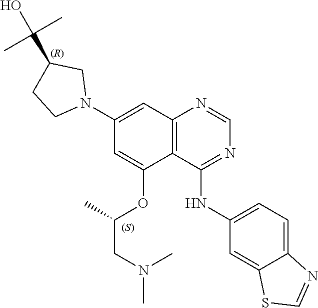 |
| 157 and 158 | 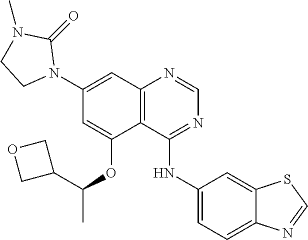 [SMILES: C[C@H](Oc1cc(N2CCN(C)C2=O)cc2ncnc(Nc3ccc4ncsc4c3)c12)C1COC1] |
| | or |
| | 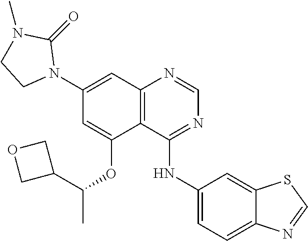 [SMILES: C[C@@H](Oc1cc(N2CCN(C)C2=O)cc2ncnc(Nc3ccc4ncsc4c3)c12)C1COC1] |
| 159 and 160 | 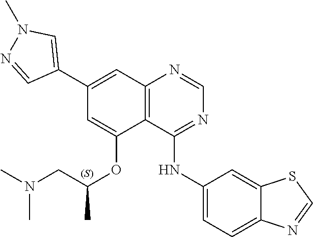 |
| | or |
| | 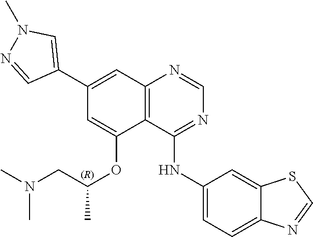 |
| 161 and 162 | 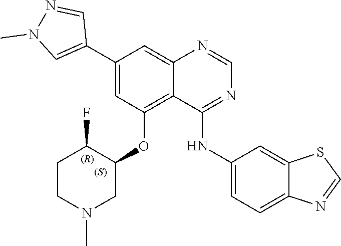 |
| | or |
| | 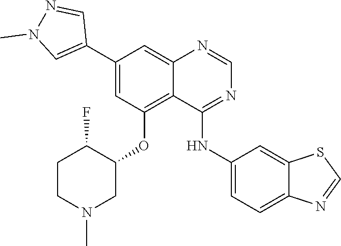 [SMILES: CN1CC[C@H](F)[C@H](Oc2cc(-c3cnn(C)c3)cc3ncnc(Nc4ccc5ncsc5c4)c23)C1] |
| 163 | 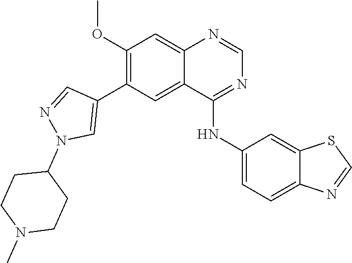 [SMILES: COc1cc2ncnc(Nc3ccc4ncsc4c3)c2cc1-c1cnn(C2CCN(C)CC2)c1] |
| 164 | 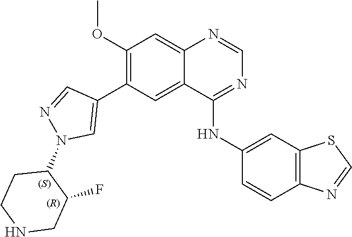 |
| | cis racemic |
| 165 | 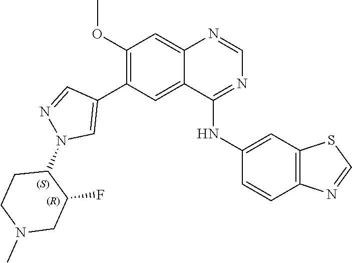 |
| | cis-racemic |
| 166 and 167 | 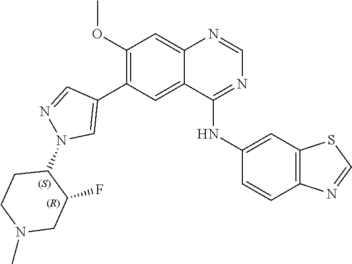 |
| | or |
| | 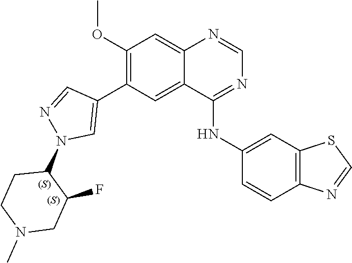 |
| 168 | 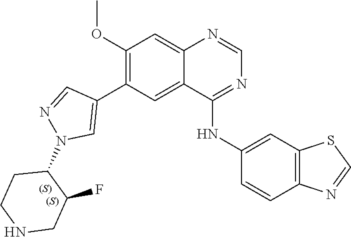 |
| | trans racemate |
| 169 | 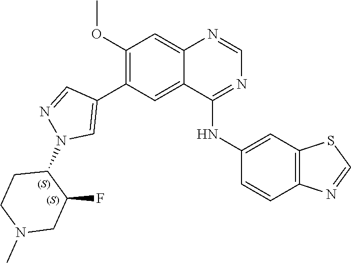 |
| | trans racemate |
| 170 and 171 | 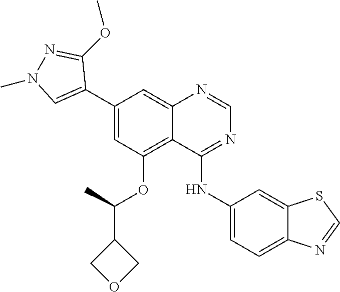 [SMILES: COc1nn(C)cc1-c1cc(O[C@H](C)C2COC2)c2c(Nc3ccc4ncsc4c3)ncnc2c1] |
| | or |
| | 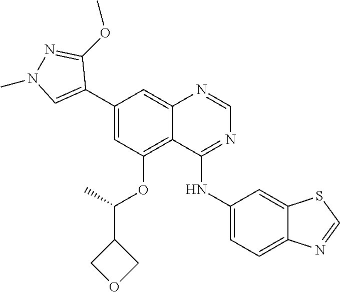 [SMILES: COc1nn(C)cc1-c1cc(O[C@@H](C)C2COC2)c2c(Nc3ccc4ncsc4c3)ncnc2c1] |
| 172 | 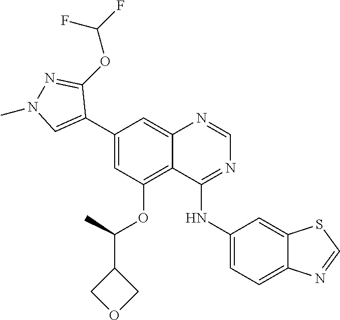 [SMILES: C[C@@H](Oc1cc(-c2cn(C)nc2OC(F)F)cc2ncnc(Nc3ccc4ncsc4c3)c12)C1COC1] |
| | or |
| 173 | 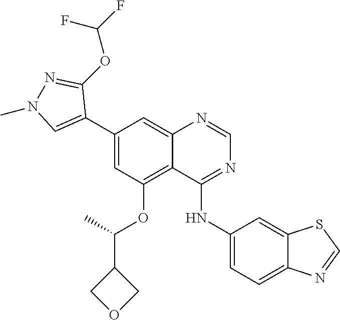 [SMILES: C[C@H](Oc1cc(-c2cn(C)nc2OC(F)F)cc2ncnc(Nc3ccc4ncsc4c3)c12)C1COC1] |
| | Or |
| | 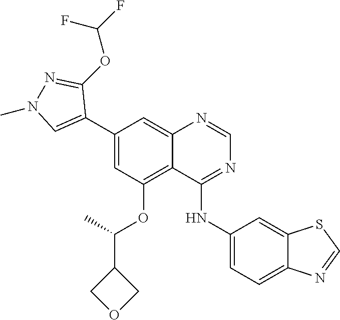 [SMILES: C[C@H](Oc1cc(-c2cn(C)nc2OC(F)F)cc2ncnc(Nc3ccc4ncsc4c3)c12)C1COC1] |
| 174 and 175 | 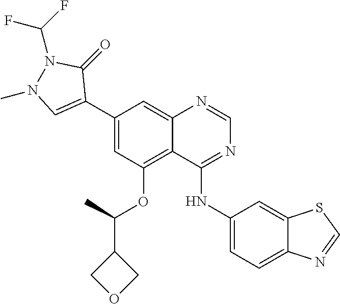 [SMILES: C[C@@H](Oc1cc(-c2cn(C)n(C(F)F)c2=O)cc2ncnc(Nc3ccc4ncsc4c3)c12)C1COC1] |
| | Or |
| | 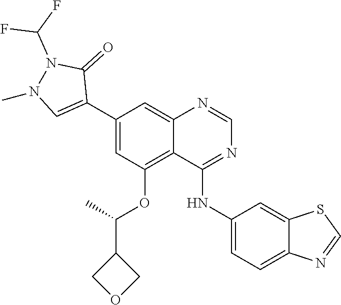 [SMILES: C[C@H](Oc1cc(-c2cn(C)n(C(F)F)c2=O)cc2ncnc(Nc3ccc4ncsc4c3)c12)C1COC1] |
| 176 | 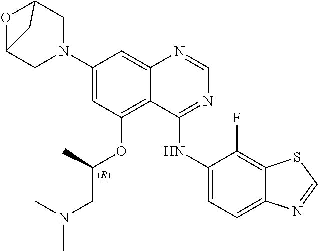 |
| 177 | 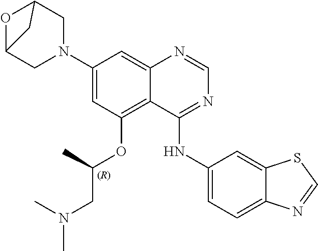 |
| 178 and 179 | 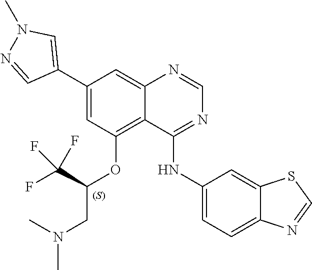 |
| | Or |
| | 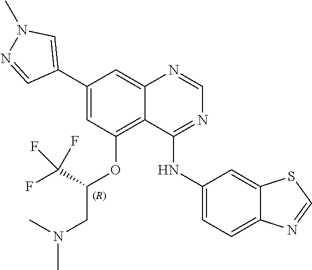 |
| 180 | 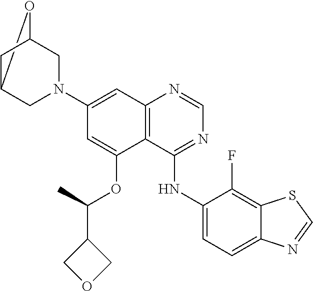 [SMILES: C[C@@H](Oc1cc(N2CC3CC(C2)O3)cc2ncnc(Nc3ccc4ncsc4c3F)c12)C1COC1] |
| 181 | 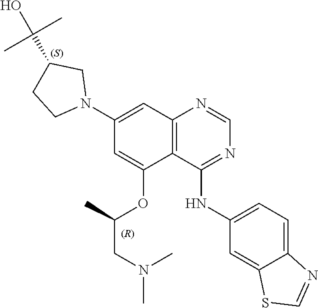 |
| 182 | 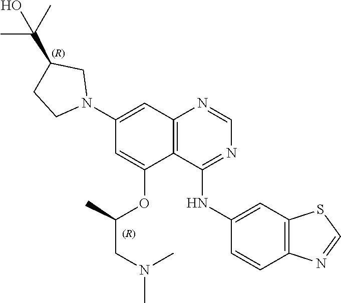 |
| 183 | 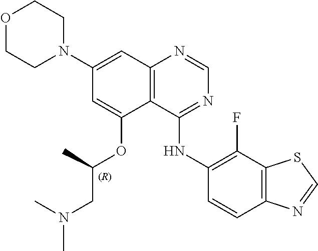 |
| 184 | 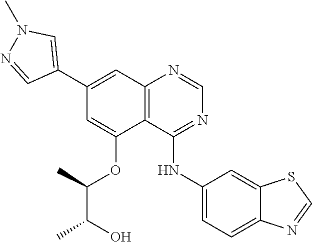 [SMILES: C[C@@H](O)[C@@H](C)Oc1cc(-c2cnn(C)c2)cc2ncnc(Nc3ccc4ncsc4c3)c12] |
| 185 | 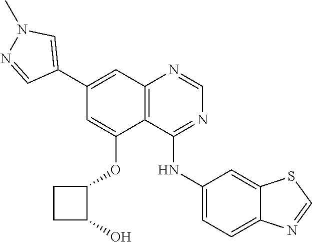 [SMILES: Cn1cc(-c2cc(O[C@H]3CC[C@H]3O)c3c(Nc4ccc5ncsc5c4)ncnc3c2)cn1] |
| 186 | 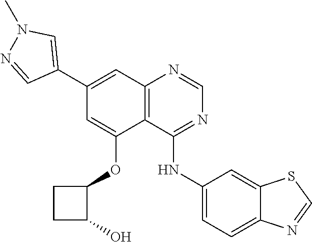 [SMILES: Cn1cc(-c2cc(O[C@@H]3CC[C@H]3O)c3c(Nc4ccc5ncsc5c4)ncnc3c2)cn1] |
| 187 | 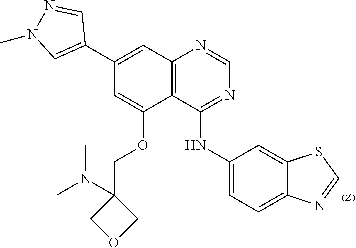 [SMILES: CN(C)C1(COc2cc(-c3cnn(C)c3)cc3ncnc(Nc4ccc5ncsc5c4)c23)COC1] |
| 188 | 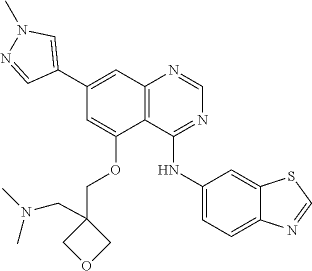 [SMILES: CN(C)CC1(COc2cc(-c3cnn(C)c3)cc3ncnc(Nc4ccc5ncsc5c4)c23)COC1] |
| 189 | 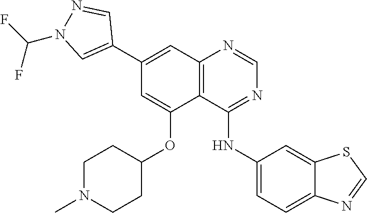 [SMILES: CN1CCC(Oc2cc(-c3cnn(C(F)F)c3)cc3ncnc(Nc4ccc5ncsc5c4)c23)CC1] |
| 190 | 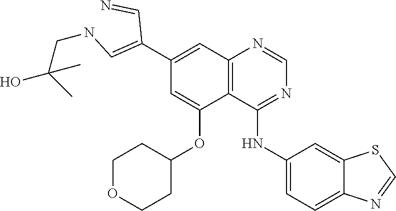 [SMILES: CC(C)(O)CN/C=C(\C=N)c1cc(OC2CCOCC2)c2c(Nc3ccc4ncsc4c3)ncnc2c1] |
| 191 | 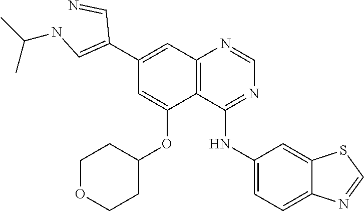 [SMILES: CC(C)N/C=C(\C=N)c1cc(OC2CCOCC2)c2c(Nc3ccc4ncsc4c3)ncnc2c1] |
| 192 | 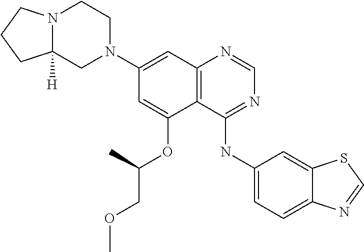 [SMILES: COC[C@@H](C)Oc1cc(N2CCN3CCC[C@@H]3C2)cc2ncnc(Nc3ccc4ncsc4c3)c12] |
| 193 | 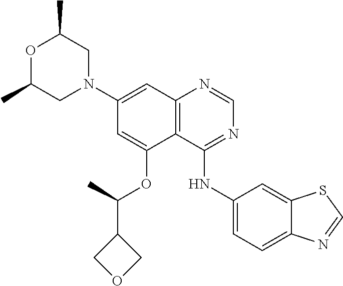 [SMILES: C[C@@H]1CN(c2cc(O[C@H](C)C3COC3)c3c(Nc4ccc5ncsc5c4)ncnc3c2)C[C@H](C)O1] |
| 194 | 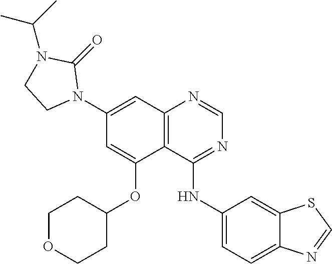 [SMILES: CC(C)N1CCN(c2cc(OC3CCOCC3)c3c(Nc4ccc5ncsc5c4)ncnc3c2)C1=O] |
| 195 | 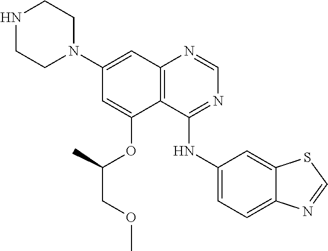 [SMILES: COC[C@@H](C)Oc1cc(N2CCNCC2)cc2ncnc(Nc3ccc4ncsc4c3)c12] |
| 196 | 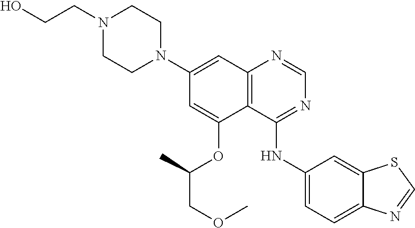 [SMILES: COC[C@@H](C)Oc1cc(N2CCN(CCO)CC2)cc2ncnc(Nc3ccc4ncsc4c3)c12] |
| 197 | 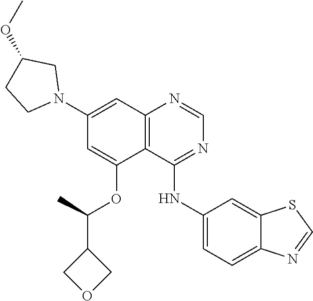 [SMILES: CO[C@H]1CCN(c2cc(O[C@H](C)C3COC3)c3c(Nc4ccc5ncsc5c4)ncnc3c2)C1] |
| 198 | 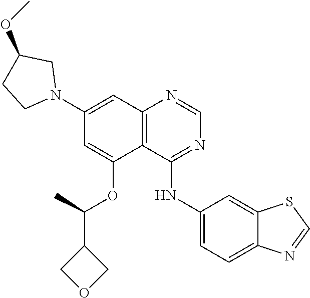 [SMILES: CO[C@@H]1CCN(c2cc(O[C@H](C)C3COC3)c3c(Nc4ccc5ncsc5c4)ncnc3c2)C1] |
| 199 | 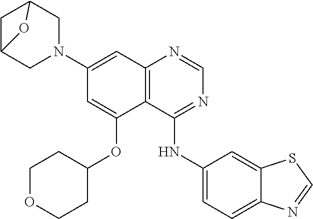 [SMILES: c1nc(Nc2ccc3ncsc3c2)c2c(OC3CCOCC3)cc(N3CC4CC(C3)O4)cc2n1] |
| 200 | 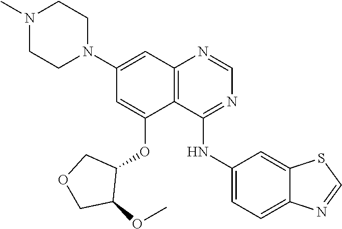 [SMILES: CO[C@H]1COC[C@@H]1Oc1cc(N2CCN(C)CC2)cc2ncnc(Nc3ccc4ncsc4c3)c12] |
| 201 | 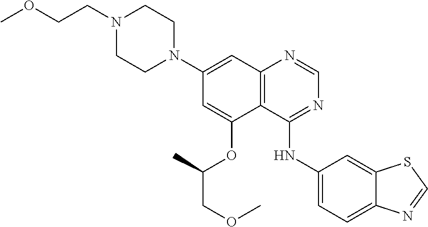 [SMILES: COCCN1CCN(c2cc(O[C@H](C)COC)c3c(Nc4ccc5ncsc5c4)ncnc3c2)CC1] |
| 202 | 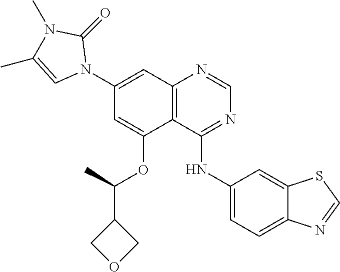 [SMILES: Cc1cn(-c2cc(O[C@H](C)C3COC3)c3c(Nc4ccc5ncsc5c4)ncnc3c2)c(=O)n1C] |
| 203 | 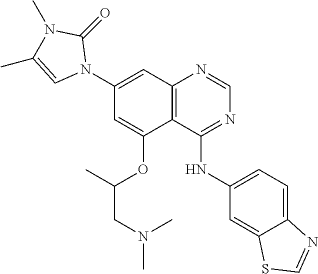 [SMILES: Cc1cn(-c2cc(OC(C)CN(C)C)c3c(Nc4ccc5ncsc5c4)ncnc3c2)c(=O)n1C] |
| 204 | 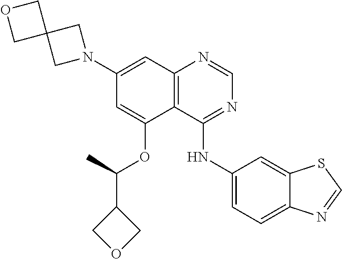 [SMILES: C[C@@H](Oc1cc(N2CC3(COC3)C2)cc2ncnc(Nc3ccc4ncsc4c3)c12)C1COC1] |
| 205 and 206 | 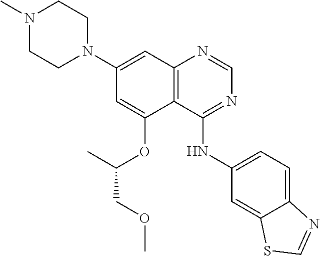 [SMILES: COC[C@H](C)Oc1cc(N2CCN(C)CC2)cc2ncnc(Nc3ccc4ncsc4c3)c12] |
| | or |
| | 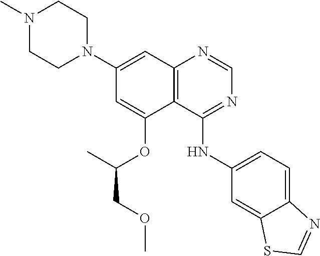 [SMILES: COC[C@@H](C)Oc1cc(N2CCN(C)CC2)cc2ncnc(Nc3ccc4ncsc4c3)c12] |
| 207 and 208 | 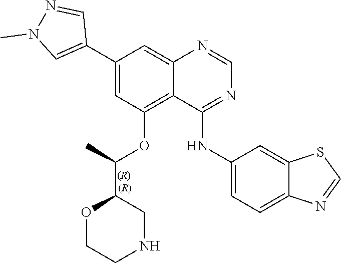 |
| | or |
| | 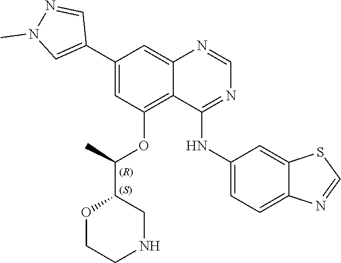 |
| 209 and 210 | 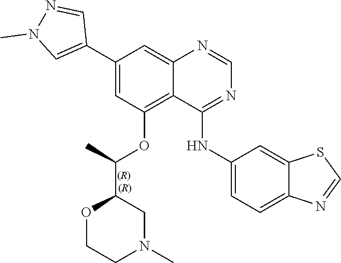 |
| | or |
| | 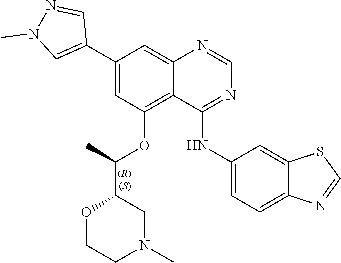 |
| 211 and 212 | 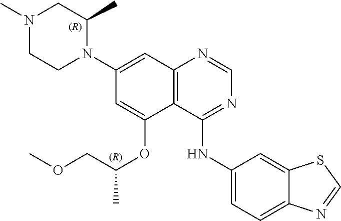 |
| | or |
| | 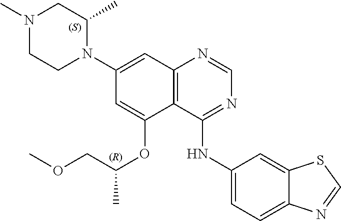 |
| 226 | 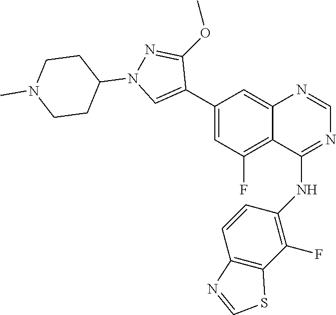 [SMILES: COc1nn(C2CCN(C)CC2)cc1-c1cc(F)c2c(Nc3ccc4ncsc4c3F)ncnc2c1] |
| 227 | 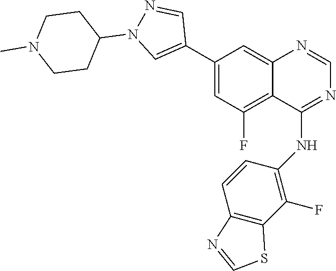 [SMILES: CN1CCC(n2cc(-c3cc(F)c4c(Nc5ccc6ncsc6c5F)ncnc4c3)cn2)CC1] |
| 240 | 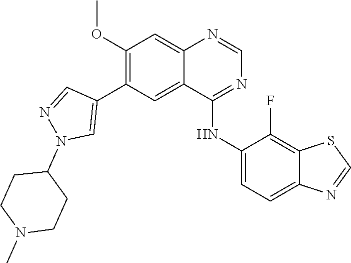 [SMILES: COc1cc2ncnc(Nc3ccc4ncsc4c3F)c2cc1-c1cnn(C2CCN(C)CC2)c1] |
| 241 | 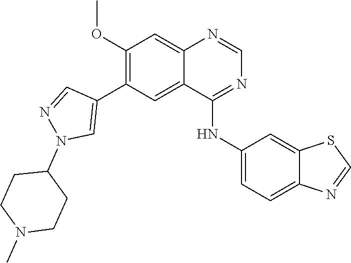 [SMILES: COc1cc2ncnc(Nc3ccc4ncsc4c3)c2cc1-c1cnn(C2CCN(C)CC2)c1] |
| 242 | 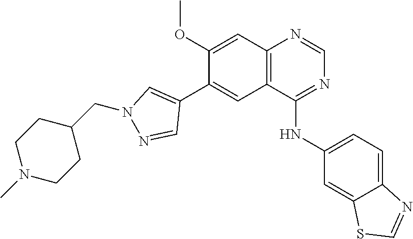 [SMILES: COc1cc2ncnc(Nc3ccc4ncsc4c3)c2cc1-c1cnn(CC2CCN(C)CC2)c1] |
| 243 | 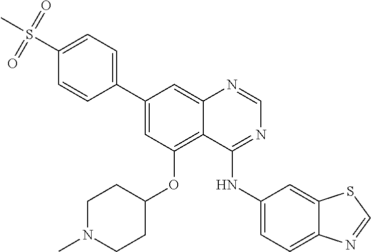 [SMILES: CN1CCC(Oc2cc(-c3ccc(S(C)(=O)=O)cc3)cc3ncnc(Nc4ccc5ncsc5c4)c23)CC1] |
| 245 | 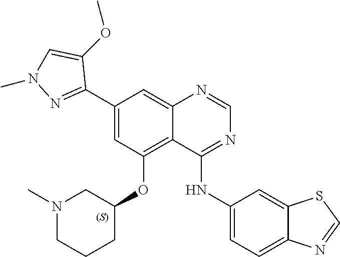 |
| 246 | 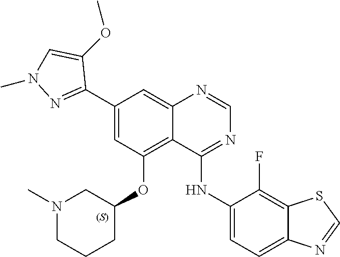 |
| 247 | 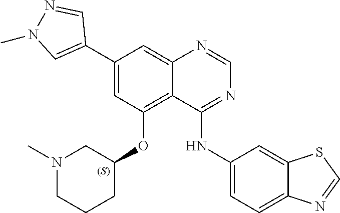 |
| 248 | 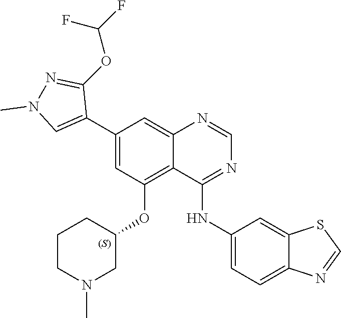 |
| 249 | 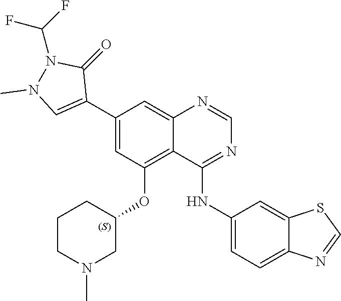 |
| 250 | 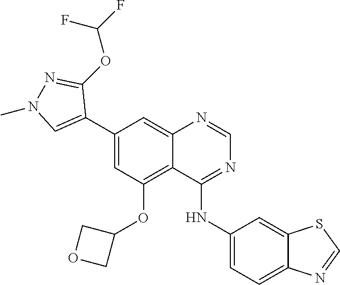 [SMILES: Cn1cc(-c2cc(OC3COC3)c3c(Nc4ccc5ncsc5c4)ncnc3c2)c(OC(F)F)n1] |
| 251 | 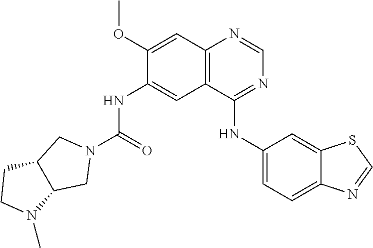 [SMILES: COc1cc2ncnc(Nc3ccc4ncsc4c3)c2cc1NC(=O)N1C[C@H]2CCN(C)[C@H]2C1] |
| 252 | 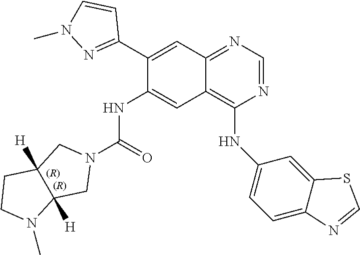 |
| 253 | 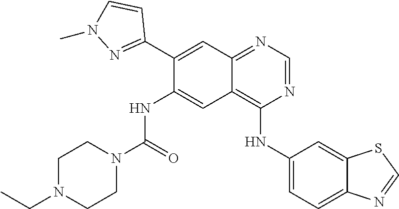 [SMILES: CCN1CCN(C(=O)Nc2cc3c(Nc4ccc5ncsc5c4)ncnc3cc2-c2ccn(C)n2)CC1] |
| 254 | 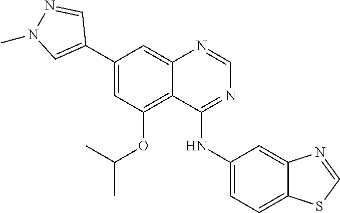 [SMILES: CC(C)Oc1cc(-c2cnn(C)c2)cc2ncnc(Nc3ccc4scnc4c3)c12] |
| 255 | 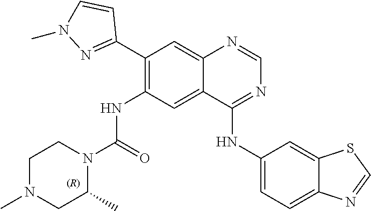 |
| 256 | 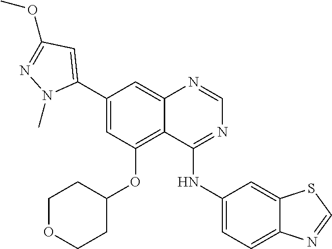 [SMILES: COc1cc(-c2cc(OC3CCOCC3)c3c(Nc4ccc5ncsc5c4)ncnc3c2)n(C)n1] |
| 257 | 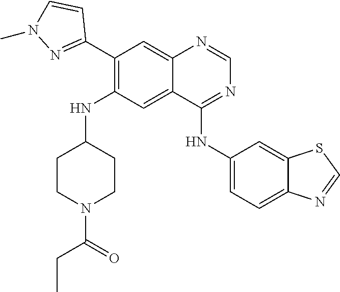 [SMILES: CCC(=O)N1CCC(Nc2cc3c(Nc4ccc5ncsc5c4)ncnc3cc2-c2ccn(C)n2)CC1] |
| 258 | 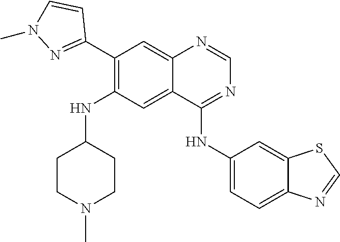 [SMILES: CN1CCC(Nc2cc3c(Nc4ccc5ncsc5c4)ncnc3cc2-c2ccn(C)n2)CC1] |
| 259 | 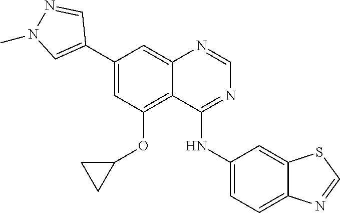 [SMILES: Cn1cc(-c2cc(OC3CC3)c3c(Nc4ccc5ncsc5c4)ncnc3c2)cn1] |
| 260 | 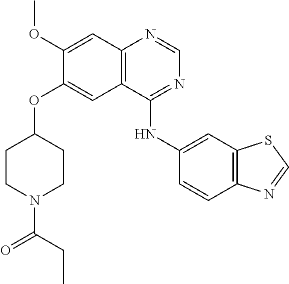 [SMILES: CCC(=O)N1CCC(Oc2cc3c(Nc4ccc5ncsc5c4)ncnc3cc2OC)CC1] |
| 261 | 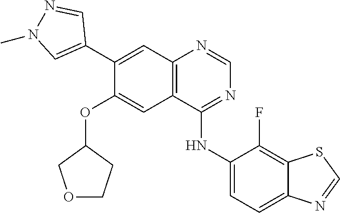 [SMILES: Cn1cc(-c2cc3ncnc(Nc4ccc5ncsc5c4F)c3cc2OC2CCOC2)cn1] |
| 262 | 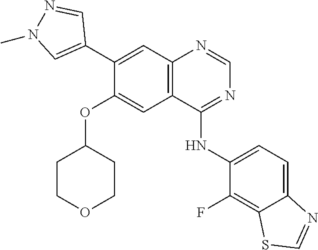 [SMILES: Cn1cc(-c2cc3ncnc(Nc4ccc5ncsc5c4F)c3cc2OC2CCOCC2)cn1] |
| 263 | 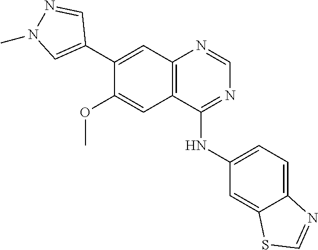 [SMILES: COc1cc2c(Nc3ccc4ncsc4c3)ncnc2cc1-c1cnn(C)c1] |
| 264 | 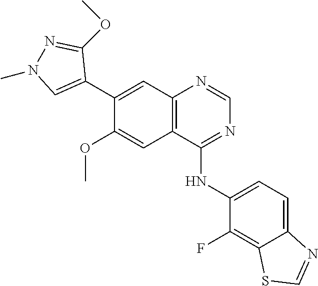 [SMILES: COc1cc2c(Nc3ccc4ncsc4c3F)ncnc2cc1-c1cn(C)nc1OC] |
| 265 | 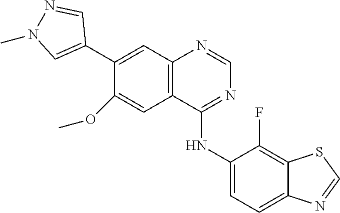 [SMILES: COc1cc2c(Nc3ccc4ncsc4c3F)ncnc2cc1-c1cnn(C)c1] |
| 266 | 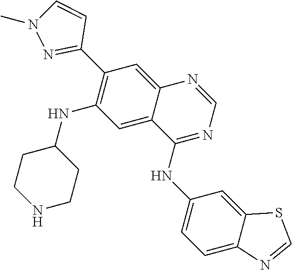 [SMILES: Cn1ccc(-c2cc3ncnc(Nc4ccc5ncsc5c4)c3cc2NC2CCNCC2)n1] |
| 270 | 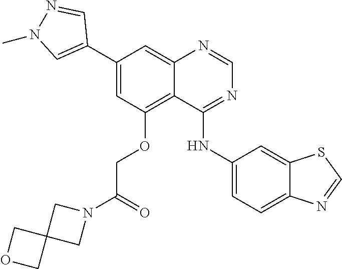 [SMILES: Cn1cc(-c2cc(OCC(=O)N3CC4(COC4)C3)c3c(Nc4ccc5ncsc5c4)ncnc3c2)cn1] |
| 271 | 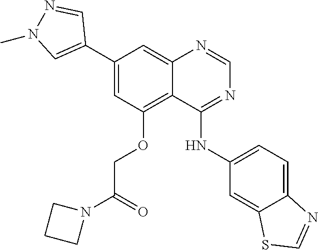 [SMILES: Cn1cc(-c2cc(OCC(=O)N3CCC3)c3c(Nc4ccc5ncsc5c4)ncnc3c2)cn1] |
| 272 | 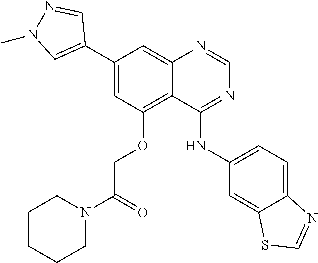 [SMILES: Cn1cc(-c2cc(OCC(=O)N3CCCCC3)c3c(Nc4ccc5ncsc5c4)ncnc3c2)cn1] |
| 273 | 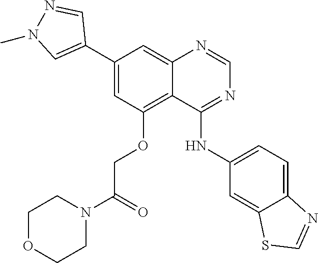 [SMILES: Cn1cc(-c2cc(OCC(=O)N3CCOCC3)c3c(Nc4ccc5ncsc5c4)ncnc3c2)cn1] |
| 274 | 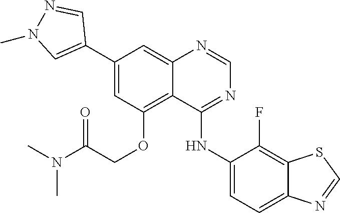 [SMILES: CN(C)C(=O)COc1cc(-c2cnn(C)c2)cc2ncnc(Nc3ccc4ncsc4c3F)c12] |
| 275 | 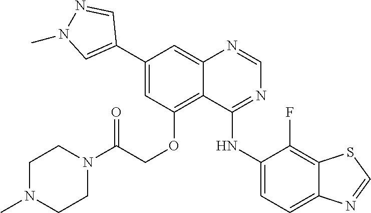 [SMILES: CN1CCN(C(=O)COc2cc(-c3cnn(C)c3)cc3ncnc(Nc4ccc5ncsc5c4F)c23)CC1] |
| 276 | 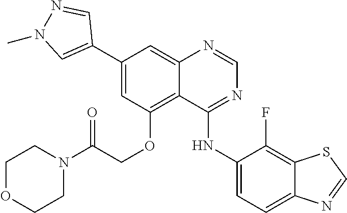 [SMILES: Cn1cc(-c2cc(OCC(=O)N3CCOCC3)c3c(Nc4ccc5ncsc5c4F)ncnc3c2)cn1] |
| 277 | 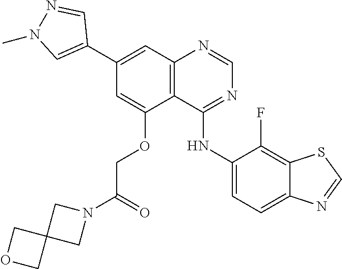 [SMILES: Cn1cc(-c2cc(OCC(=O)N3CC4(COC4)C3)c3c(Nc4ccc5ncsc5c4F)ncnc3c2)cn1] |
| 278 | 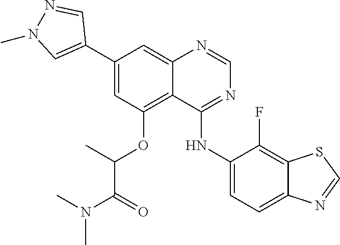 [SMILES: CC(Oc1cc(-c2cnn(C)c2)cc2ncnc(Nc3ccc4ncsc4c3F)c12)C(=O)N(C)C] |
| 279 and 280 | 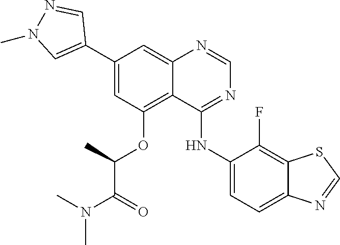 [SMILES: C[C@@H](Oc1cc(-c2cnn(C)c2)cc2ncnc(Nc3ccc4ncsc4c3F)c12)C(=O)N(C)C] |
| | or |
| | 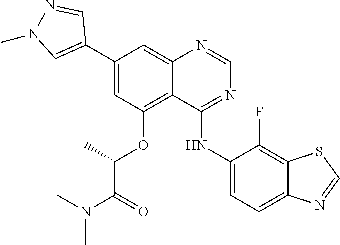 [SMILES: C[C@H](Oc1cc(-c2cnn(C)c2)cc2ncnc(Nc3ccc4ncsc4c3F)c12)C(=O)N(C)C] |
| 281 | 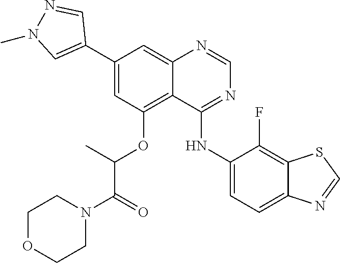 [SMILES: CC(Oc1cc(-c2cnn(C)c2)cc2ncnc(Nc3ccc4ncsc4c3F)c12)C(=O)N1CCOCC1] |
| 282 and 283 | 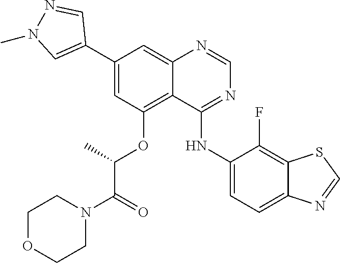 [SMILES: C[C@H](Oc1cc(-c2cnn(C)c2)cc2ncnc(Nc3ccc4ncsc4c3F)c12)C(=O)N1CCOCC1] |
| | or |
| | 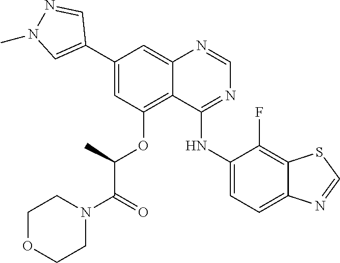 [SMILES: C[C@@H](Oc1cc(-c2cnn(C)c2)cc2ncnc(Nc3ccc4ncsc4c3F)c12)C(=O)N1CCOCC1] |
| 284 | 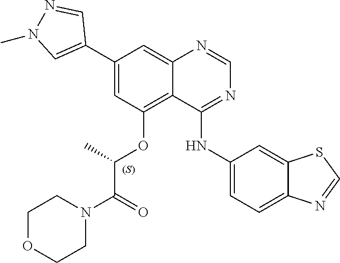 |
| 285 | 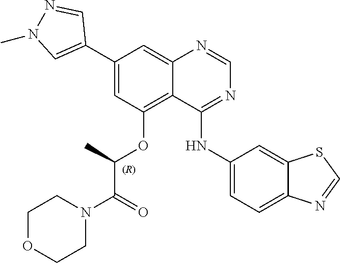 |
| 286 | 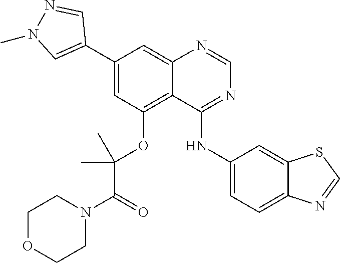 [SMILES: Cn1cc(-c2cc(OC(C)(C)C(=O)N3CCOCC3)c3c(Nc4ccc5ncsc5c4)ncnc3c2)cn1] |
| 287 and 288 | 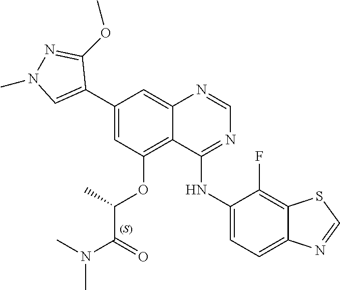 |
| | or |
| | 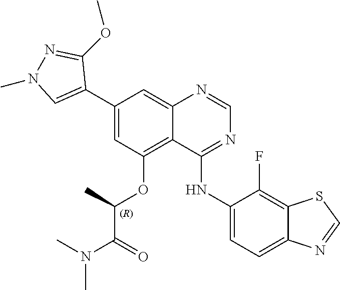 |
| 289 | 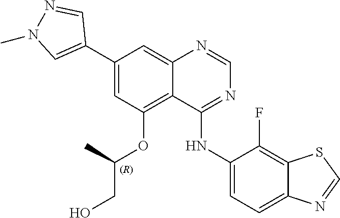 |
| | or |
| 290 | 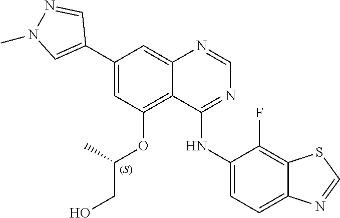 |
| 291 and 292 | 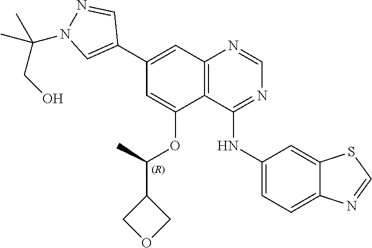 |
| | or |
| | 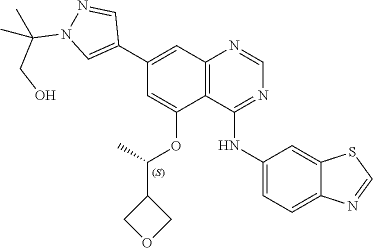 |
| 293 and 294 | 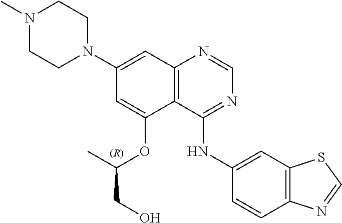 |
| | or |
| | 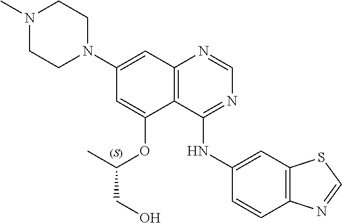 |
| 295 and 296 | 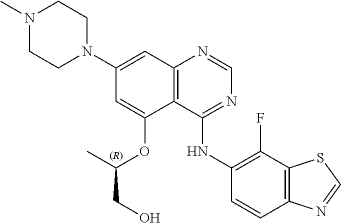 |
| | or |
| | 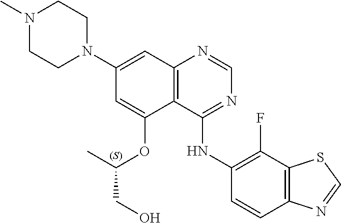 |
| 297 | 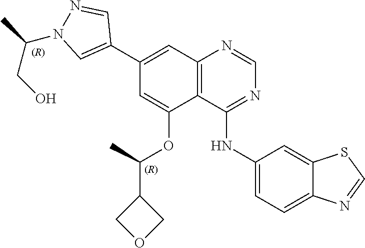 |
| | or |
| 298 | 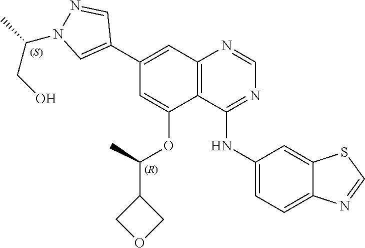 |
| 299 and 300 | 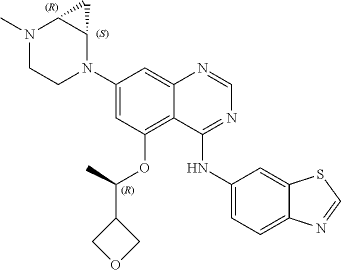 |
| | or |
| | 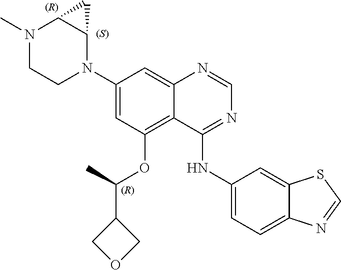 |
| 301 | 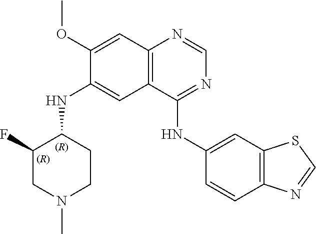 |
| 305 | 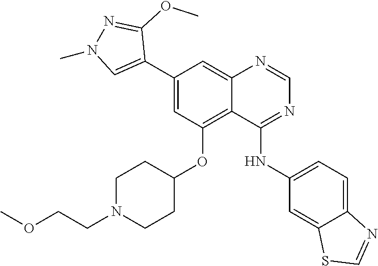 [SMILES: COCCN1CCC(Oc2cc(-c3cn(C)nc3OC)cc3ncnc(Nc4ccc5ncsc5c4)c23)CC1] |
| 306 | 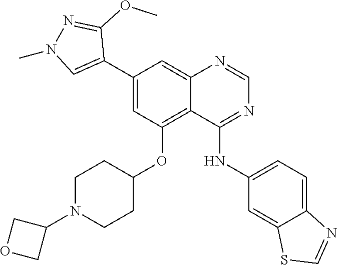 [SMILES: COc1nn(C)cc1-c1cc(OC2CCN(C3COC3)CC2)c2c(Nc3ccc4ncsc4c3)ncnc2c1] |
| 307 | 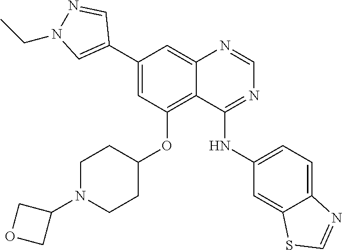 [SMILES: CCn1cc(-c2cc(OC3CCN(C4COC4)CC3)c3c(Nc4ccc5ncsc5c4)ncnc3c2)cn1] |
| 308 | 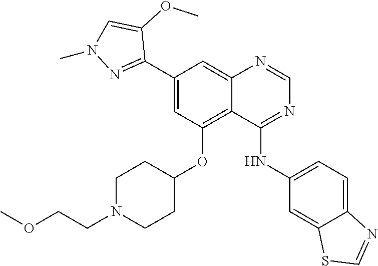 [SMILES: COCCN1CCC(Oc2cc(-c3nn(C)cc3OC)cc3ncnc(Nc4ccc5ncsc5c4)c23)CC1] |
| 309 | 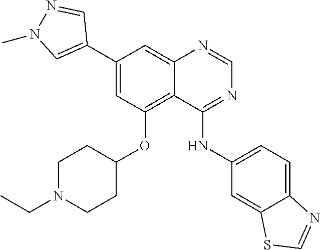 [SMILES: CCN1CCC(Oc2cc(-c3cnn(C)c3)cc3ncnc(Nc4ccc5ncsc5c4)c23)CC1] |
| 310 | 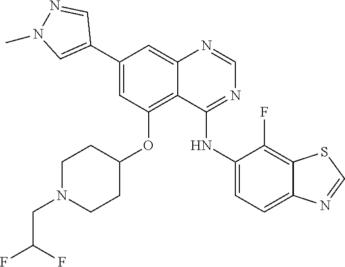 [SMILES: Cn1cc(-c2cc(OC3CCN(CC(F)F)CC3)c3c(Nc4ccc5ncsc5c4F)ncnc3c2)cn1] |
| 311 | 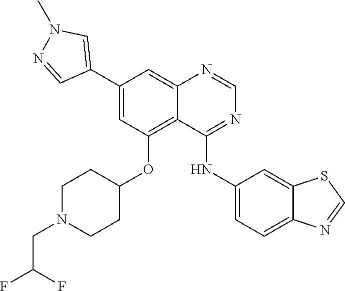 [SMILES: Cn1cc(-c2cc(OC3CCN(CC(F)F)CC3)c3c(Nc4ccc5ncsc5c4)ncnc3c2)cn1] |
| 312 | 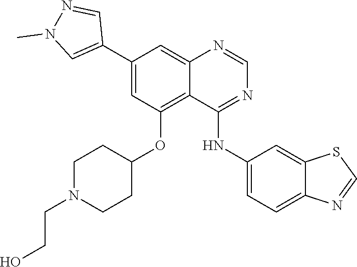 [SMILES: Cn1cc(-c2cc(OC3CCN(CCO)CC3)c3c(Nc4ccc5ncsc5c4)ncnc3c2)cn1] |
| 313 | 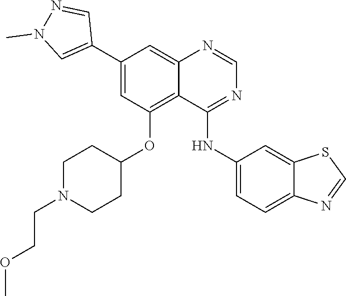 [SMILES: COCCN1CCC(Oc2cc(-c3cnn(C)c3)cc3ncnc(Nc4ccc5ncsc5c4)c23)CC1] |
| 314 | 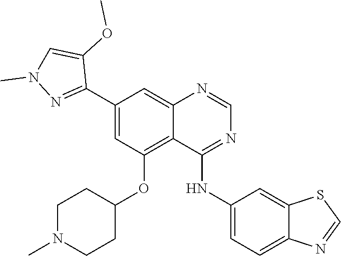 [SMILES: COc1cn(C)nc1-c1cc(OC2CCN(C)CC2)c2c(Nc3ccc4ncsc4c3)ncnc2c1] |
| 315 | 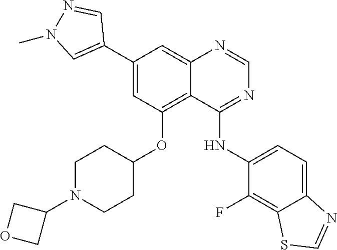 [SMILES: Cn1cc(-c2cc(OC3CCN(C4COC4)CC3)c3c(Nc4ccc5ncsc5c4F)ncnc3c2)cn1] |
| 316 | 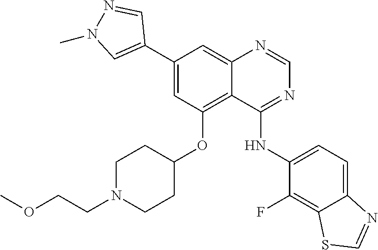 [SMILES: COCCN1CCC(Oc2cc(-c3cnn(C)c3)cc3ncnc(Nc4ccc5ncsc5c4F)c23)CC1] |
| 317 | 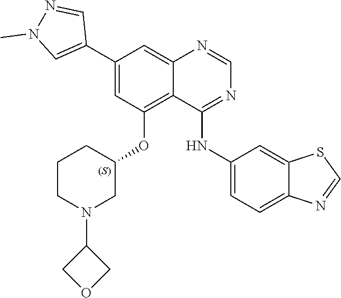 |
| 318 | 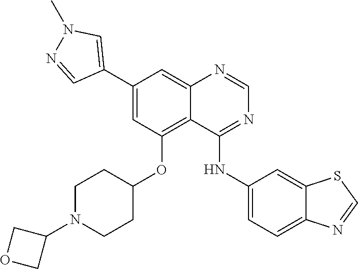 [SMILES: Cn1cc(-c2cc(OC3CCN(C4COC4)CC3)c3c(Nc4ccc5ncsc5c4)ncnc3c2)cn1] |
| 319 | 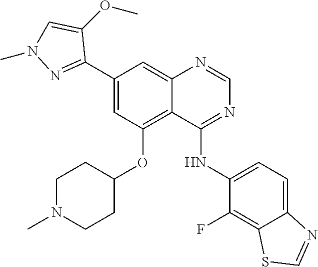 [SMILES: COc1cn(C)nc1-c1cc(OC2CCN(C)CC2)c2c(Nc3ccc4ncsc4c3F)ncnc2c1] |
| 320 | 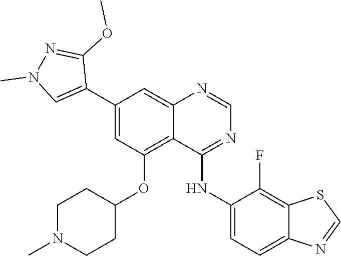 [SMILES: COc1nn(C)cc1-c1cc(OC2CCN(C)CC2)c2c(Nc3ccc4ncsc4c3F)ncnc2c1] |
| 321 and 322 | 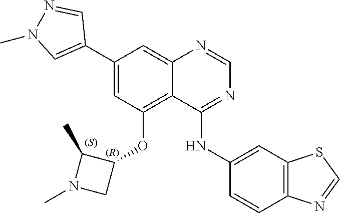 |
| | or |
| | 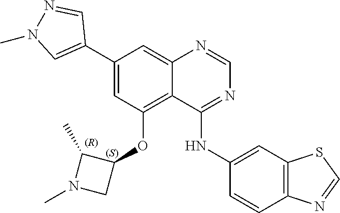 |
| 325 and 326 | 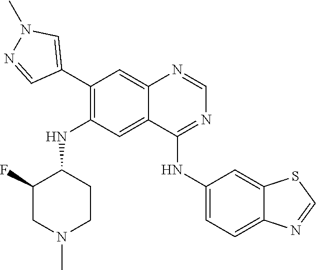 [SMILES: CN1CC[C@@H](Nc2cc3c(Nc4ccc5ncsc5c4)ncnc3cc2-c2cnn(C)c2)[C@H](F)C1] |
| | or |
| | 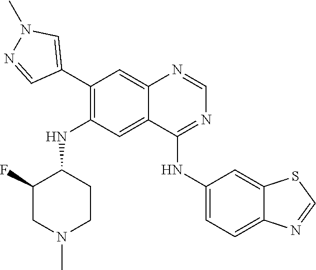 [SMILES: CN1CC[C@@H](Nc2cc3c(Nc4ccc5ncsc5c4)ncnc3cc2-c2cnn(C)c2)[C@H](F)C1] |
| 327 and 328 | 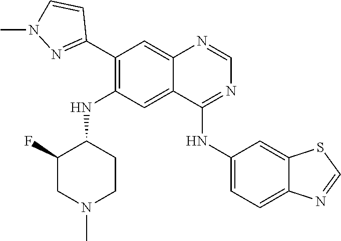 [SMILES: CN1CC[C@@H](Nc2cc3c(Nc4ccc5ncsc5c4)ncnc3cc2-c2ccn(C)n2)[C@H](F)C1] |
| | or |
| | 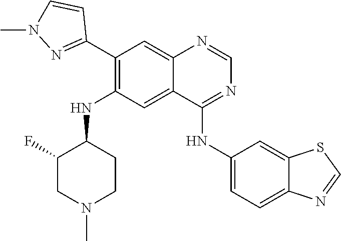 [SMILES: CN1CC[C@H](Nc2cc3c(Nc4ccc5ncsc5c4)ncnc3cc2-c2ccn(C)n2)[C@@H](F)C1] |
| 329 | 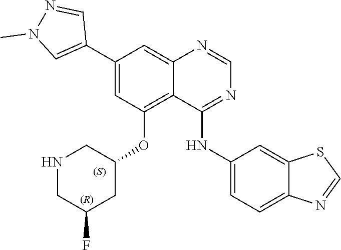 |
| | cis-racemic |
| 331 | 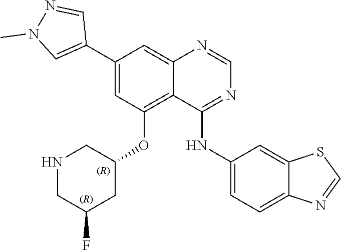 |
| | trans racemic |
| 332 and 333 | 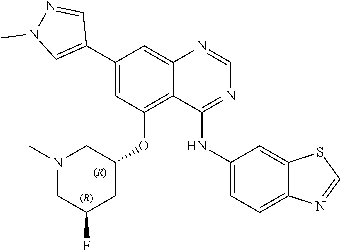 |
| | or |
| | 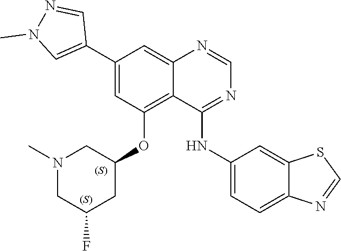 |
| 334 and 335 | 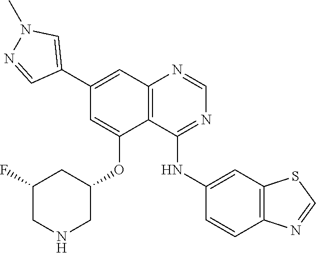 [SMILES: Cn1cc(-c2cc(O[C@@H]3CNC[C@H](F)C3)c3c(Nc4ccc5ncsc5c4)ncnc3c2)cn1] |
| | or |
| | 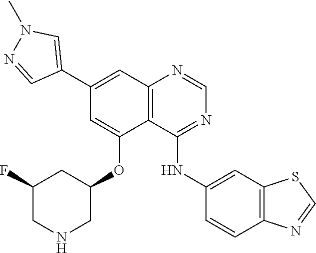 [SMILES: Cn1cc(-c2cc(O[C@H]3CNC[C@@H](F)C3)c3c(Nc4ccc5ncsc5c4)ncnc3c2)cn1] |
| 336 | 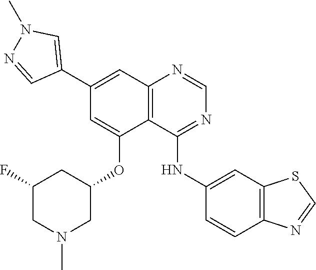 [SMILES: CN1C[C@H](F)C[C@H](Oc2cc(-c3cnn(C)c3)cc3ncnc(Nc4ccc5ncsc5c4)c23)C1] |
| | or |
| 337 | 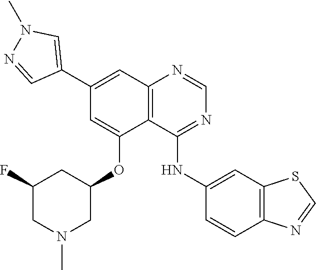 [SMILES: CN1C[C@@H](F)C[C@@H](Oc2cc(-c3cnn(C)c3)cc3ncnc(Nc4ccc5ncsc5c4)c23)C1] |
| | or |
| | 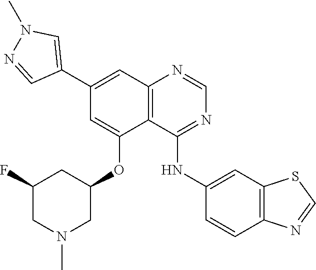 [SMILES: CN1C[C@@H](F)C[C@@H](Oc2cc(-c3cnn(C)c3)cc3ncnc(Nc4ccc5ncsc5c4)c23)C1] |
| 339 | 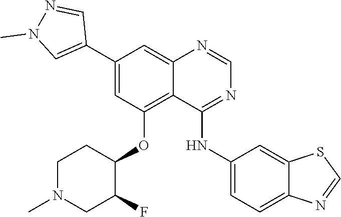 [SMILES: CN1CC[C@@H](Oc2cc(-c3cnn(C)c3)cc3ncnc(Nc4ccc5ncsc5c4)c23)[C@@H](F)C1] |
| | cis racemate |
| 340 and 341 | 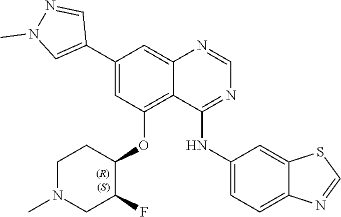 |
| | or |
| | 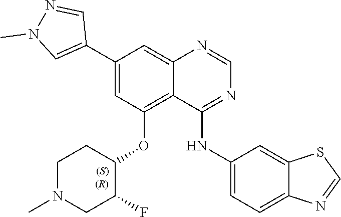 |
| 343 | 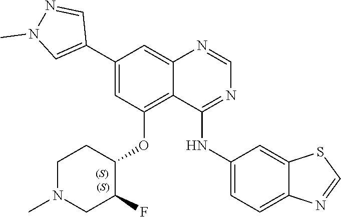 |
| | trans racemic |
| 344 | 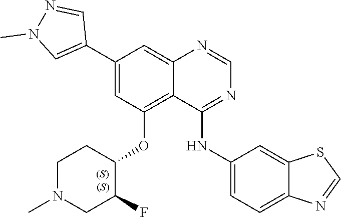 |
| | or |
| 345 | 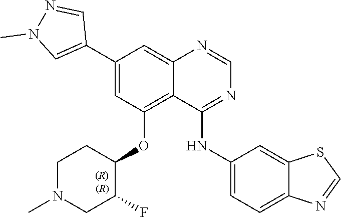 |
| 346 and 347 | 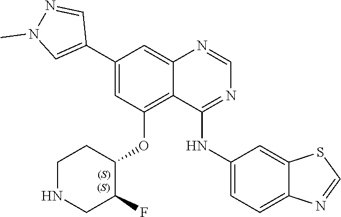 |
| | or |
| | 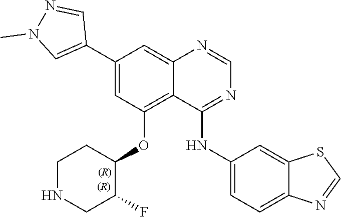 |
| 348 | 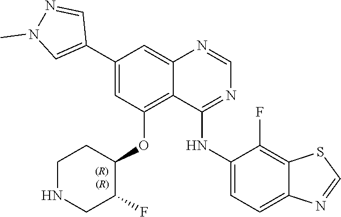 |
| 349 | 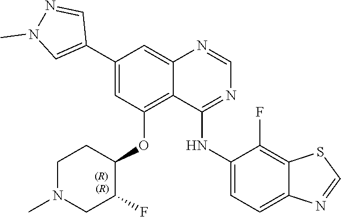 |
| 350 | 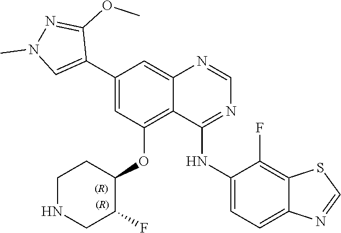 |
| 351 | 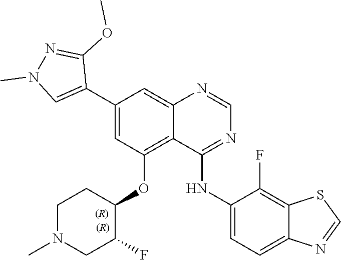 |
| 352 | 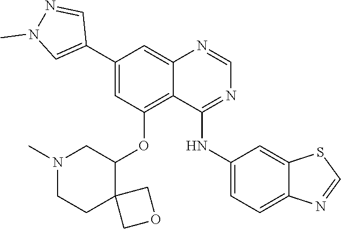 [SMILES: CN1CCC2(COC2)C(Oc2cc(-c3cnn(C)c3)cc3ncnc(Nc4ccc5ncsc5c4)c23)C1] |
| 353 and 354 | 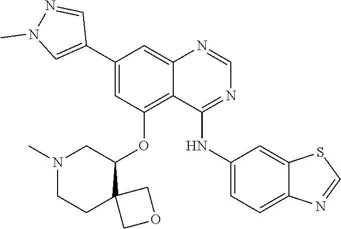 [SMILES: CN1CCC2(COC2)[C@H](Oc2cc(-c3cnn(C)c3)cc3ncnc(Nc4ccc5ncsc5c4)c23)C1] |
| | or |
| | 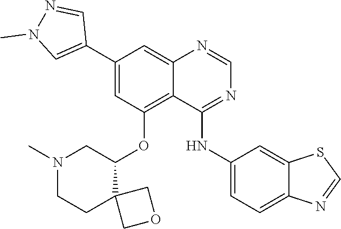 [SMILES: CN1CCC2(COC2)[C@@H](Oc2cc(-c3cnn(C)c3)cc3ncnc(Nc4ccc5ncsc5c4)c23)C1] |
| 356 | 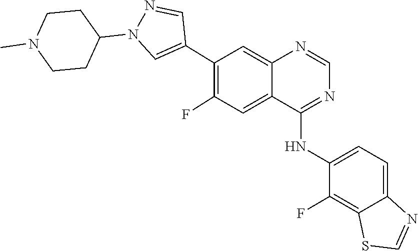 [SMILES: CN1CCC(n2cc(-c3cc4ncnc(Nc5ccc6ncsc6c5F)c4cc3F)cn2)CC1] |
| 358 and 359 | 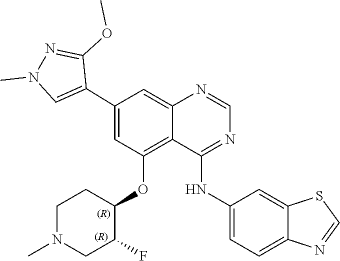 |
| | or |
| | 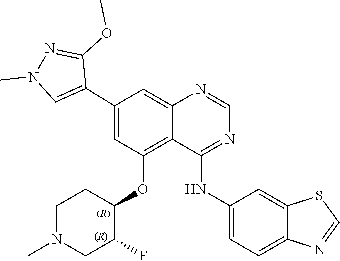 |
| 360 and 361 | 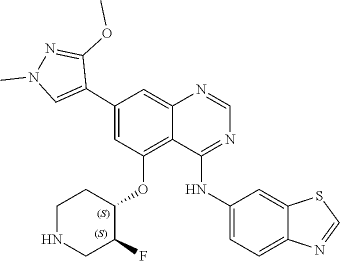 |
| | or |
| | 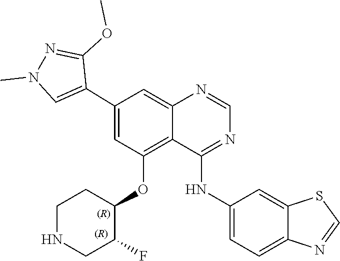 |
| 362 | 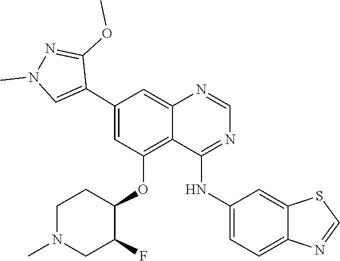 [SMILES: COc1nn(C)cc1-c1cc(O[C@@H]2CCN(C)C[C@@H]2F)c2c(Nc3ccc4ncsc4c3)ncnc2c1] |
| 363 | 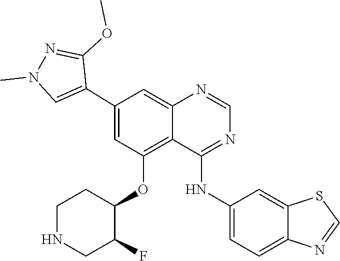 [SMILES: COc1nn(C)cc1-c1cc(O[C@@H]2CCNC[C@@H]2F)c2c(Nc3ccc4ncsc4c3)ncnc2c1] |
| 364 | 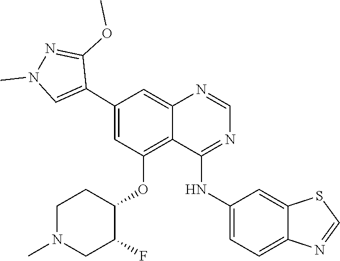 [SMILES: COc1nn(C)cc1-c1cc(O[C@H]2CCN(C)C[C@H]2F)c2c(Nc3ccc4ncsc4c3)ncnc2c1] |
| 365 | 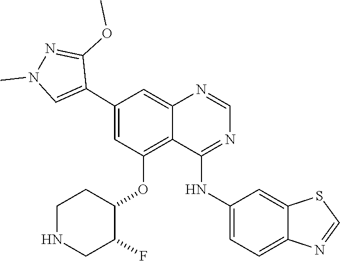 |
| 366 | 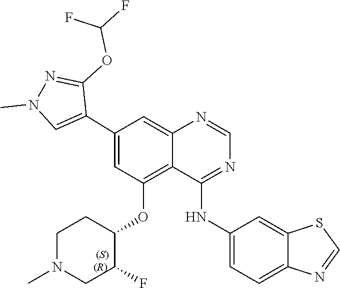 |
| 367 | 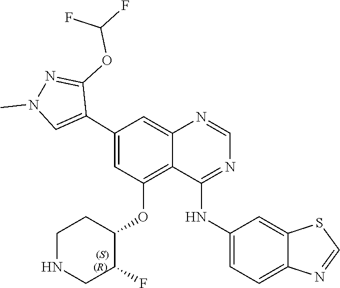 |
| 368 and 369 | 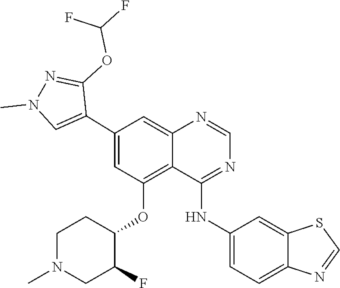 [SMILES: CN1CC[C@H](Oc2cc(-c3cn(C)nc3OC(F)F)cc3ncnc(Nc4ccc5ncsc5c4)c23)[C@@H](F)C1] |
| | or |
| | 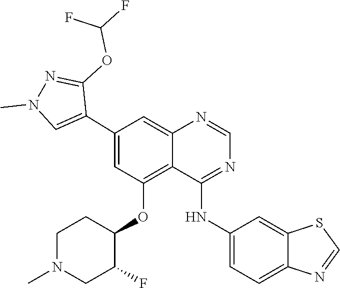 [SMILES: CN1CC[C@@H](Oc2cc(-c3cn(C)nc3OC(F)F)cc3ncnc(Nc4ccc5ncsc5c4)c23)[C@H](F)C1] |
| 370 | 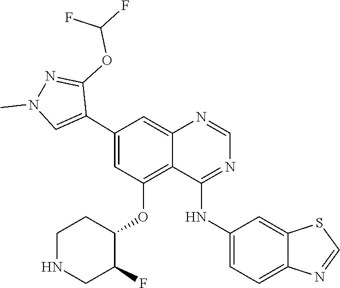 [SMILES: Cn1cc(-c2cc(O[C@H]3CCNC[C@@H]3F)c3c(Nc4ccc5ncsc5c4)ncnc3c2)c(OC(F)F)n1] |
| | trans racemic |
| 371 | 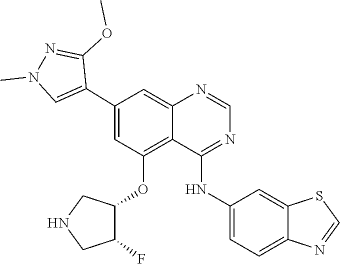 [SMILES: COc1nn(C)cc1-c1cc(O[C@H]2CNC[C@H]2F)c2c(Nc3ccc4ncsc4c3)ncnc2c1] |
| | cis racemic |
| 373 | 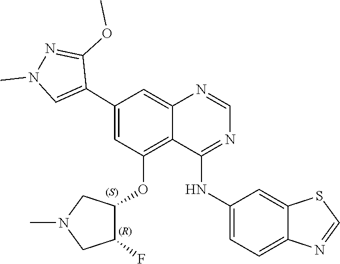 |
| | or |
| 374 | 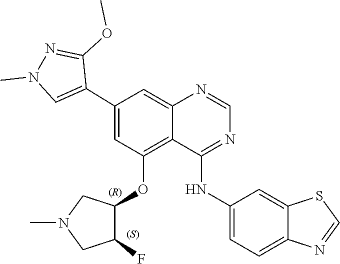 |
| 375 | 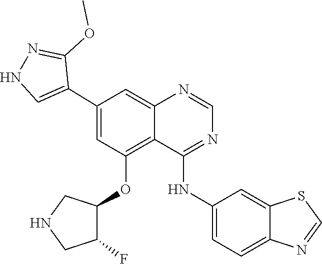 [SMILES: COc1n[nH]cc1-c1cc(O[C@@H]2CNC[C@H]2F)c2c(Nc3ccc4ncsc4c3)ncnc2c1] |
| | trans racemic |
| 376 | 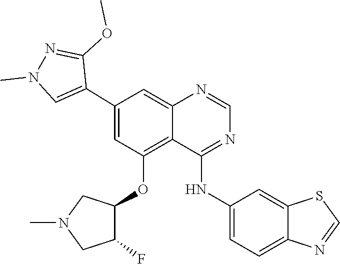 [SMILES: COc1nn(C)cc1-c1cc(O[C@@H]2CN(C)C[C@H]2F)c2c(Nc3ccc4ncsc4c3)ncnc2c1] |
| | trans racemic |
| 377 and 378 | 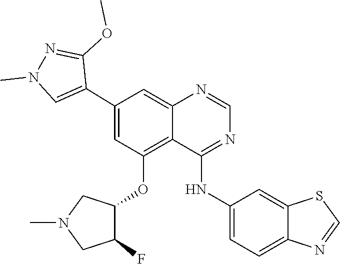 [SMILES: COc1nn(C)cc1-c1cc(O[C@H]2CN(C)C[C@@H]2F)c2c(Nc3ccc4ncsc4c3)ncnc2c1] |
| | or |
| | 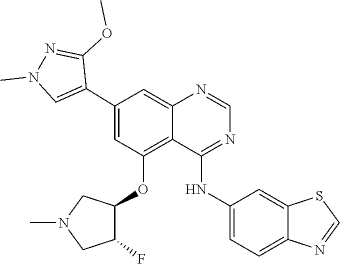 [SMILES: COc1nn(C)cc1-c1cc(O[C@@H]2CN(C)C[C@H]2F)c2c(Nc3ccc4ncsc4c3)ncnc2c1] |
| 379 | 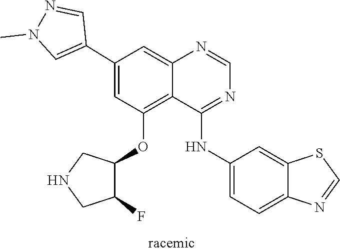 [SMILES: Cn1cc(-c2cc(O[C@@H]3CNC[C@@H]3F)c3c(Nc4ccc5ncsc5c4)ncnc3c2)cn1] |
| 380 and 381 | 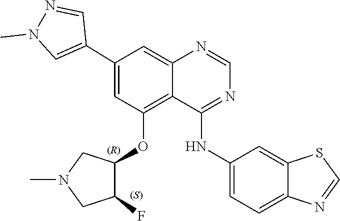 |
| | or |
| | 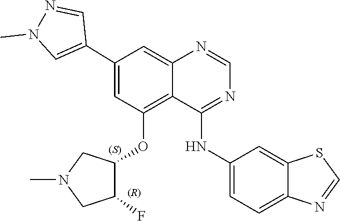 |
| 382 | 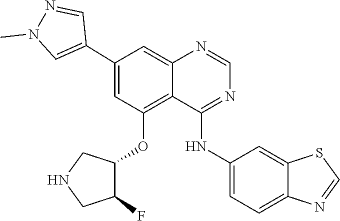 [SMILES: Cn1cc(-c2cc(O[C@H]3CNC[C@@H]3F)c3c(Nc4ccc5ncsc5c4)ncnc3c2)cn1] |
| 383 | 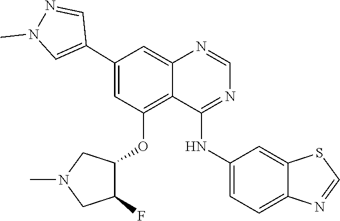 [SMILES: CN1C[C@H](Oc2cc(-c3cnn(C)c3)cc3ncnc(Nc4ccc5ncsc5c4)c23)[C@@H](F)C1] |
| 384 and 385 | 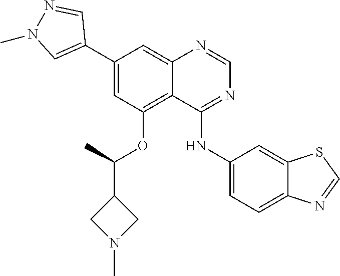 [SMILES: C[C@@H](Oc1cc(-c2cnn(C)c2)cc2ncnc(Nc3ccc4ncsc4c3)c12)C1CN(C)C1] |
| | or |
| | 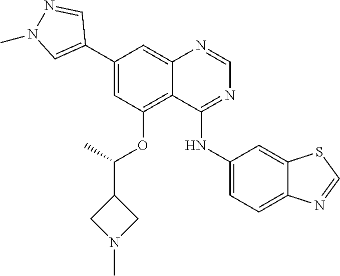 [SMILES: C[C@H](Oc1cc(-c2cnn(C)c2)cc2ncnc(Nc3ccc4ncsc4c3)c12)C1CN(C)C1] |
| 386 | 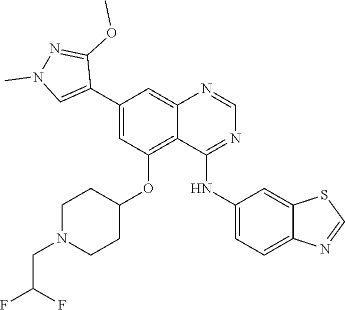 [SMILES: COc1nn(C)cc1-c1cc(OC2CCN(CC(F)F)CC2)c2c(Nc3ccc4ncsc4c3)ncnc2c1] |
| 387 and 388 | 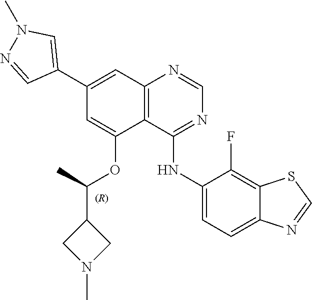 |
| | or |
| | 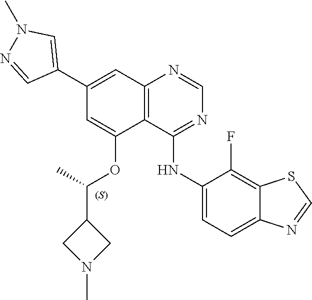 |
| 389 and 390 | 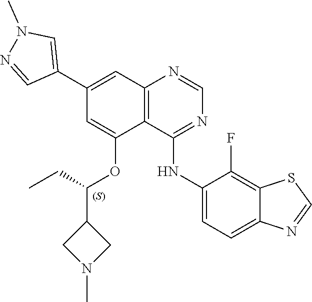 |
| | or |
| | 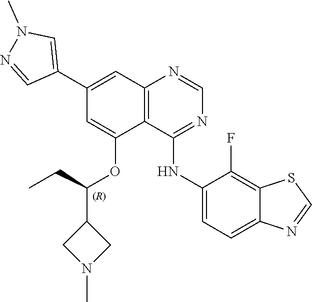 |
| 393 | 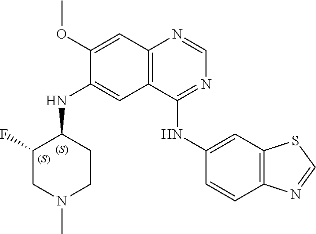 |
| 394 | 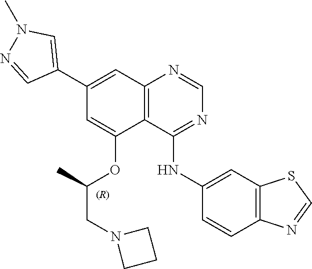 |
| 395 | 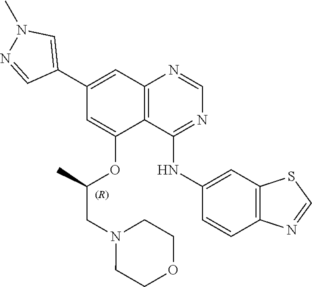 |
| 396 | 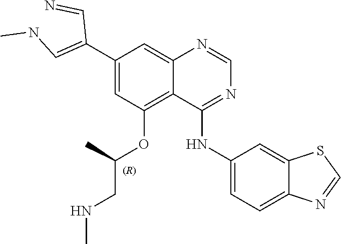 |
| 397 | 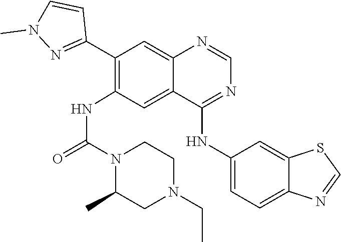 [SMILES: CCN1CCN(C(=O)Nc2cc3c(Nc4ccc5ncsc5c4)ncnc3cc2-c2ccn(C)n2)[C@H](C)C1] |
| 398 | 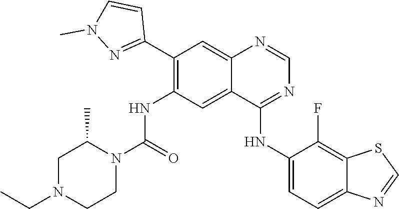 [SMILES: CCN1CCN(C(=O)Nc2cc3c(Nc4ccc5ncsc5c4F)ncnc3cc2-c2ccn(C)n2)[C@@H](C)C1] |
| 399 | 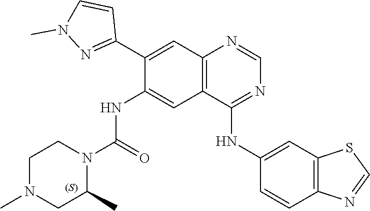 |
| 400 | 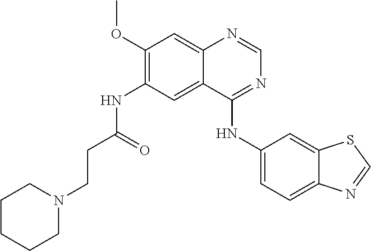 [SMILES: COc1cc2ncnc(Nc3ccc4ncsc4c3)c2cc1NC(=O)CCN1CCCCC1] |
| 401 | 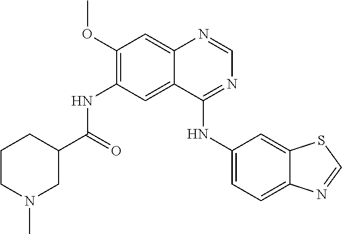 [SMILES: COc1cc2ncnc(Nc3ccc4ncsc4c3)c2cc1NC(=O)C1CCCN(C)C1] |
| 402 | 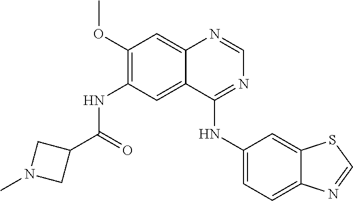 [SMILES: COc1cc2ncnc(Nc3ccc4ncsc4c3)c2cc1NC(=O)C1CN(C)C1] |
| 405 | 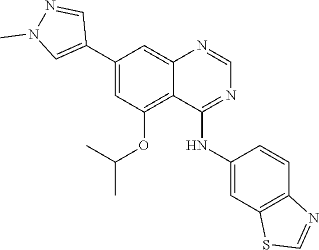 [SMILES: CC(C)Oc1cc(-c2cnn(C)c2)cc2ncnc(Nc3ccc4ncsc4c3)c12] |
| 406 | 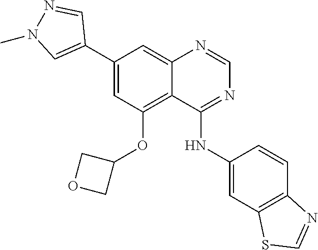 [SMILES: Cn1cc(-c2cc(OC3COC3)c3c(Nc4ccc5ncsc5c4)ncnc3c2)cn1] |
| 407 | 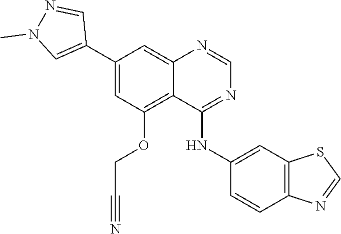 [SMILES: Cn1cc(-c2cc(OCC#N)c3c(Nc4ccc5ncsc5c4)ncnc3c2)cn1] |
| 409 | 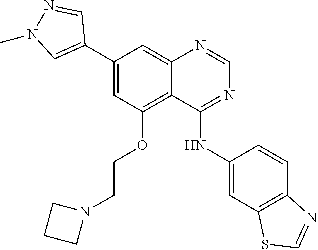 [SMILES: Cn1cc(-c2cc(OCCN3CCC3)c3c(Nc4ccc5ncsc5c4)ncnc3c2)cn1] |
| 410 | 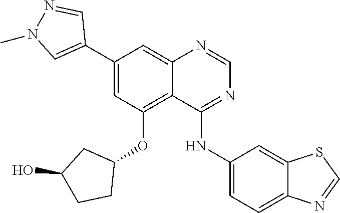 [SMILES: Cn1cc(-c2cc(O[C@@H]3CC[C@@H](O)C3)c3c(Nc4ccc5ncsc5c4)ncnc3c2)cn1] |
| 411 | 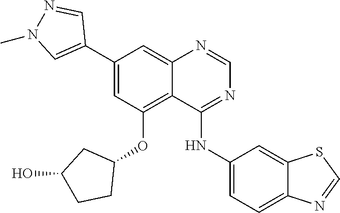 [SMILES: Cn1cc(-c2cc(O[C@@H]3CC[C@H](O)C3)c3c(Nc4ccc5ncsc5c4)ncnc3c2)cn1] |
| 412 | 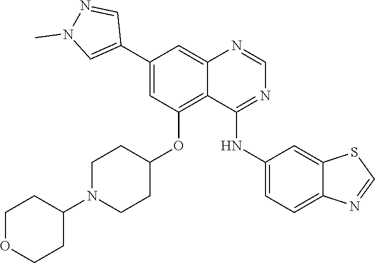 [SMILES: Cn1cc(-c2cc(OC3CCN(C4CCOCC4)CC3)c3c(Nc4ccc5ncsc5c4)ncnc3c2)cn1] |
| 413 | 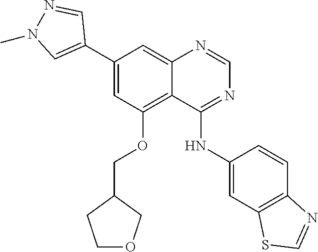 [SMILES: Cn1cc(-c2cc(OCC3CCOC3)c3c(Nc4ccc5ncsc5c4)ncnc3c2)cn1] |
| 414 | 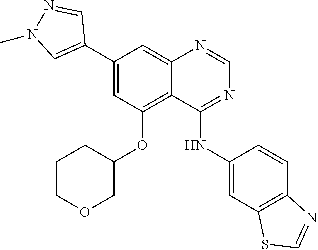 [SMILES: Cn1cc(-c2cc(OC3CCCOC3)c3c(Nc4ccc5ncsc5c4)ncnc3c2)cn1] |
| 415 | 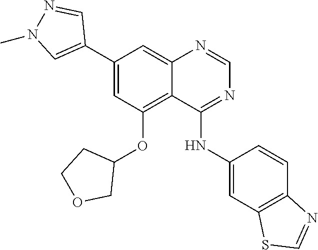 [SMILES: Cn1cc(-c2cc(OC3CCOC3)c3c(Nc4ccc5ncsc5c4)ncnc3c2)cn1] |
| A03 | 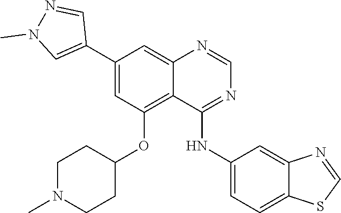 [SMILES: CN1CCC(Oc2cc(-c3cnn(C)c3)cc3ncnc(Nc4ccc5scnc5c4)c23)CC1] |
| A05 and A06 | 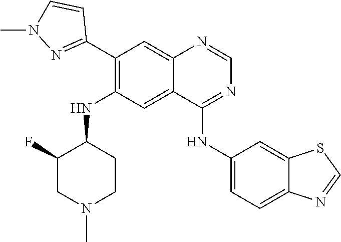 [SMILES: CN1CC[C@H](Nc2cc3c(Nc4ccc5ncsc5c4)ncnc3cc2-c2ccn(C)n2)[C@H](F)C1] |
| | or |
| | 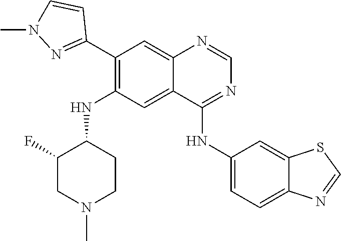 [SMILES: CN1CC[C@@H](Nc2cc3c(Nc4ccc5ncsc5c4)ncnc3cc2-c2ccn(C)n2)[C@@H](F)C1] |
| A07 | 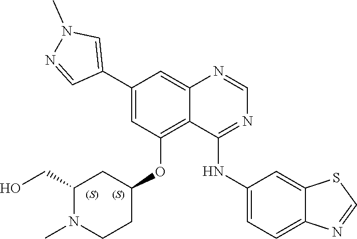 |
| A08 | 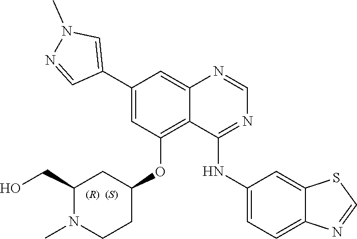 |
| A09 | 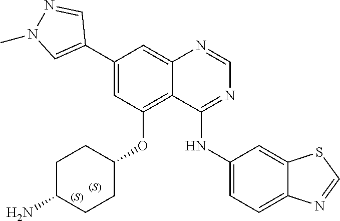 |
| A10 | |
| A11 | 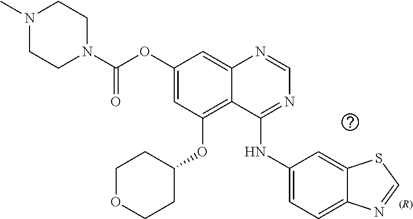 |
| A16 | |
| A17 | |
| A18 | |
| A19 and A20 | |
| A22 and A23 | 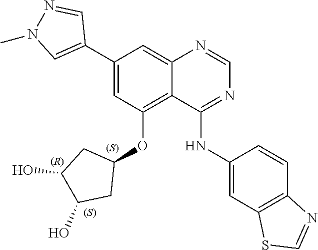 |
| | OR |
| | 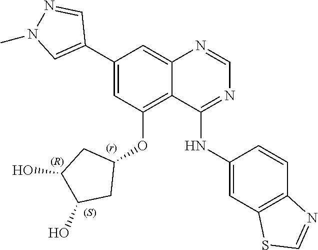 |
| A24 | 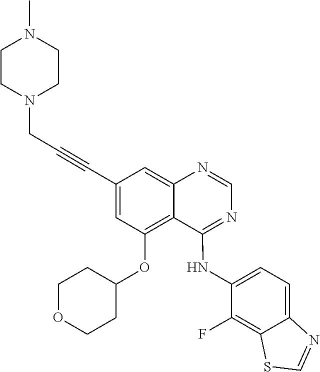 [SMILES: CN1CCN(CC#Cc2cc(OC3CCOCC3)c3c(Nc4ccc5ncsc5c4F)ncnc3c2)CC1] |
| A25 | 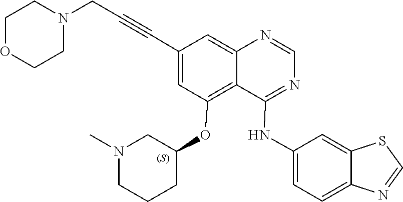 |
| A26 | 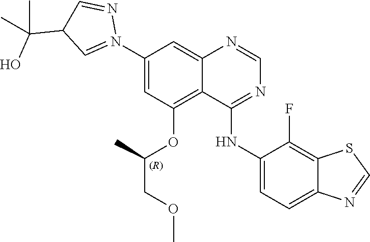 |
| A27 and A28 | 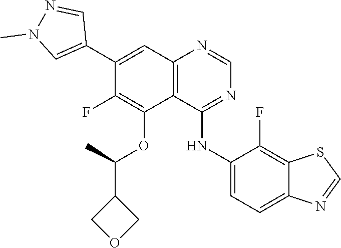 [SMILES: C[C@@H](Oc1c(F)c(-c2cnn(C)c2)cc2ncnc(Nc3ccc4ncsc4c3F)c12)C1COC1] |
| | OR |
| | 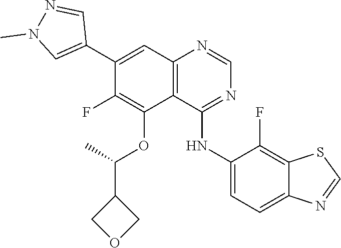 [SMILES: C[C@H](Oc1c(F)c(-c2cnn(C)c2)cc2ncnc(Nc3ccc4ncsc4c3F)c12)C1COC1] |
| A29 and A30 | 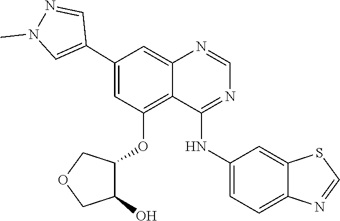 [SMILES: Cn1cc(-c2cc(O[C@H]3COC[C@@H]3O)c3c(Nc4ccc5ncsc5c4)ncnc3c2)cn1] |
| | or |
| | 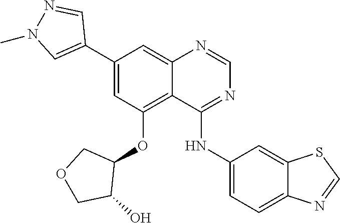 [SMILES: Cn1cc(-c2cc(O[C@@H]3COC[C@H]3O)c3c(Nc4ccc5ncsc5c4)ncnc3c2)cn1] |
| A31 and A32 | 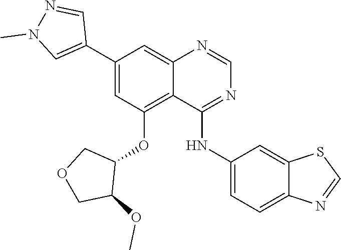 [SMILES: CO[C@H]1COC[C@@H]1Oc1cc(-c2cnn(C)c2)cc2ncnc(Nc3ccc4ncsc4c3)c12] |
| | OR |
| | 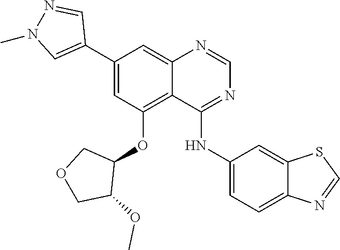 [SMILES: CO[C@@H]1COC[C@H]1Oc1cc(-c2cnn(C)c2)cc2ncnc(Nc3ccc4ncsc4c3)c12] |
| A33 and A34 | 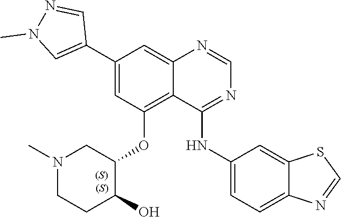 |
| | OR |
| | 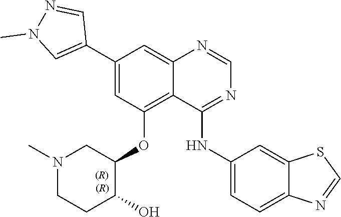 |
| A35 and A36 | 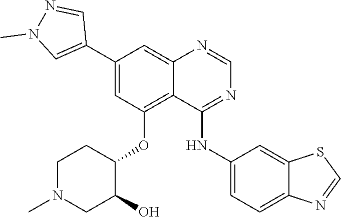 [SMILES: CN1CC[C@H](Oc2cc(-c3cnn(C)c3)cc3ncnc(Nc4ccc5ncsc5c4)c23)[C@@H](O)C1] |
| | OR |
| | 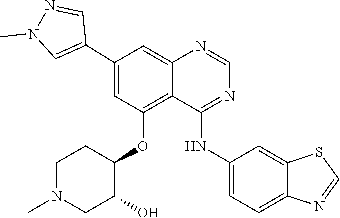 [SMILES: CN1CC[C@@H](Oc2cc(-c3cnn(C)c3)cc3ncnc(Nc4ccc5ncsc5c4)c23)[C@H](O)C1] |
| A37 and A38 | 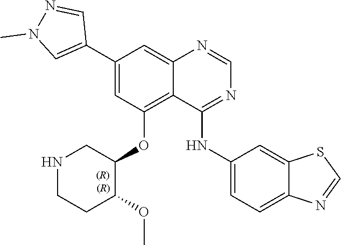 |
| | OR |
| | 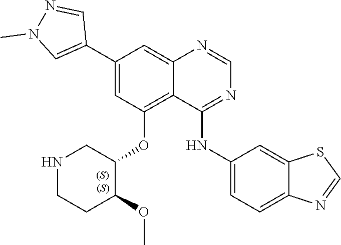 |
| A39 and A40 | 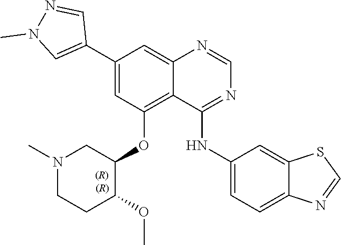 |
| | OR |
| | 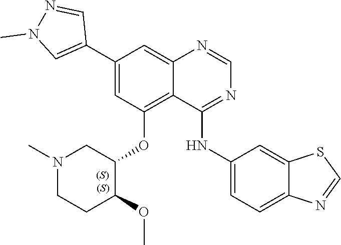 |
| A41 and A42 | 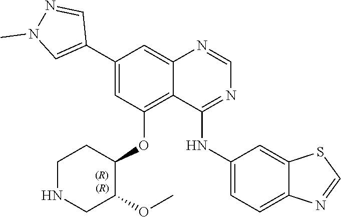 |
| | OR |
| | 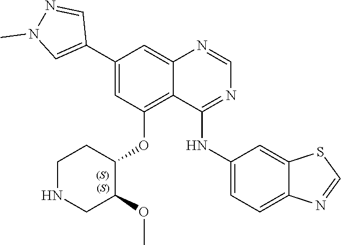 |
| A43 and A44 | 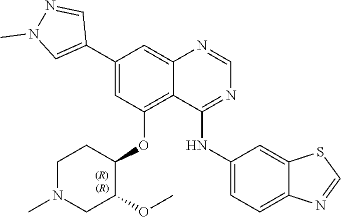 |
| | OR |
| | 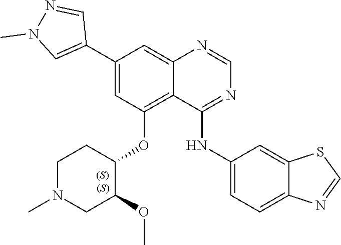 |
| A45 | 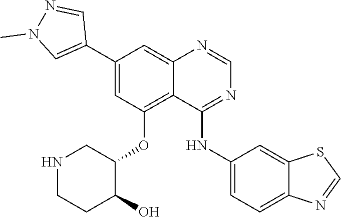 [SMILES: Cn1cc(-c2cc(O[C@H]3CNCC[C@@H]3O)c3c(Nc4ccc5ncsc5c4)ncnc3c2)cn1] |
| | trans-racemate |
| A47 | 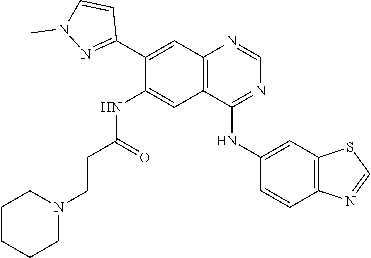 [SMILES: Cn1ccc(-c2cc3ncnc(Nc4ccc5ncsc5c4)c3cc2NC(=O)CCN2CCCCC2)n1] |
| A48 and A49 | 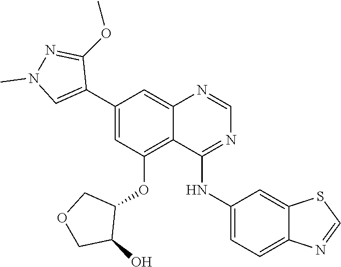 [SMILES: COc1nn(C)cc1-c1cc(O[C@H]2COC[C@@H]2O)c2c(Nc3ccc4ncsc4c3)ncnc2c1] |
| | OR |
| | 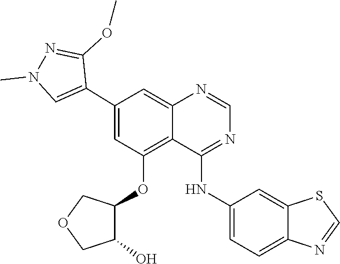 [SMILES: COc1nn(C)cc1-c1cc(O[C@@H]2COC[C@H]2O)c2c(Nc3ccc4ncsc4c3)ncnc2c1] |
| A50 | 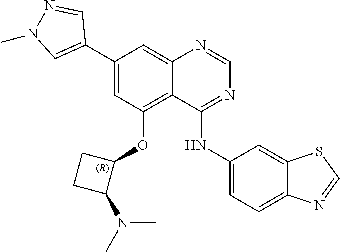 |
| | cis-racemic |
| A51 and A52 | 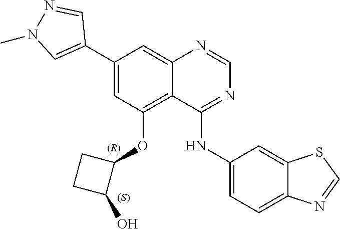 |
| | OR |
| | 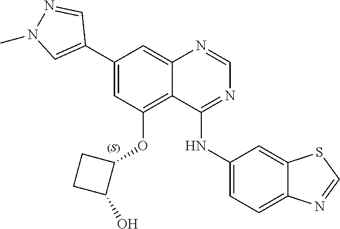 |
| A53 and A54 | 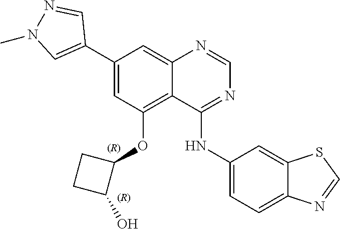 |
| | OR |
| | 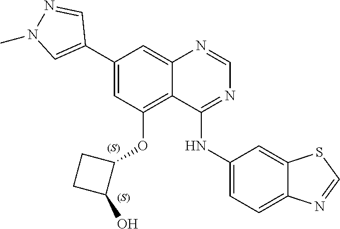 |
| A55 | 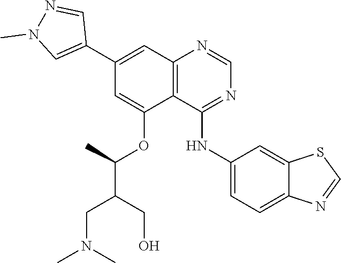 [SMILES: C[C@@H](Oc1cc(-c2cnn(C)c2)cc2ncnc(Nc3ccc4ncsc4c3)c12)C(CO)CN(C)C] |
| A56 | 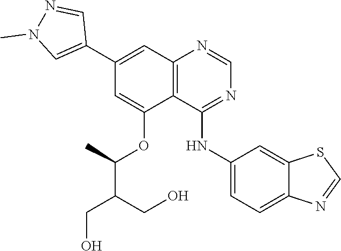 [SMILES: C[C@@H](Oc1cc(-c2cnn(C)c2)cc2ncnc(Nc3ccc4ncsc4c3)c12)C(CO)CO] |
| A57 | 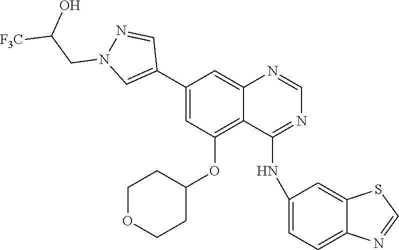 [SMILES: OC(Cn1cc(-c2cc(OC3CCOCC3)c3c(Nc4ccc5ncsc5c4)ncnc3c2)cn1)C(F)(F)F] |
| A58 | 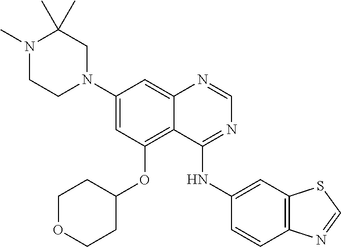 [SMILES: CN1CCN(c2cc(OC3CCOCC3)c3c(Nc4ccc5ncsc5c4)ncnc3c2)CC1(C)C] |
| A59 | 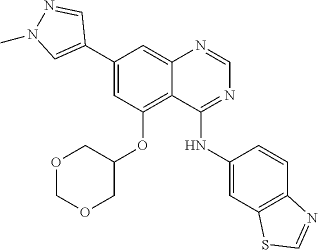 [SMILES: Cn1cc(-c2cc(OC3COCOC3)c3c(Nc4ccc5ncsc5c4)ncnc3c2)cn1] |
| A60 | 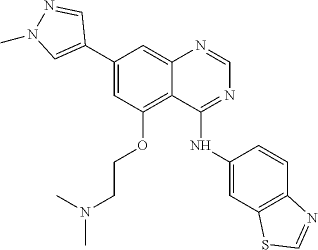 [SMILES: CN(C)CCOc1cc(-c2cnn(C)c2)cc2ncnc(Nc3ccc4ncsc4c3)c12] |

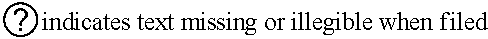

In some embodiments, the present disclosure provides a compound according to structural formula (I), (II), (III), (III-A) or (IV) or any one of the compounds of disclosed in the examples (including intermediates) and Table 1, or a pharmaceutically acceptable salt thereof, wherein one or more hydrogen is replaced with deuterium.

The term “pharmaceutically-acceptable salt” refers to a pharmaceutical salt that is, within the scope of sound medical judgment, suitable for use in contact with the tissues of humans and lower animals without undue toxicity, irritation, and allergic response, and is commensurate with a reasonable benefit/risk ratio. Pharmaceutically acceptable salts are well known in the art. For example, S. M. Berge et al. describes pharmacologically acceptable salts in J. Pharm. Sci., 1977, 66, 1-19.

Included in the present teachings are pharmaceutically acceptable salts of the compounds disclosed herein. Compounds having basic groups can form pharmaceutically acceptable salts with pharmaceutically acceptable acid(s). Suitable pharmaceutically acceptable acid addition salts of the compounds described herein include salts of inorganic acids (such as hydrochloric, hydrobromic, phosphoric, metaphosphoric, nitric, and sulfuric acids) and of organic acids (such as acetic, benzenesulfonic, benzoic, ethanesulfonic, methanesulfonic, and succinic acids). Compounds of the present teachings with acidic groups such as carboxylic acids can form pharmaceutically acceptable salts with pharmaceutically acceptable base(s). Suitable pharmaceutically acceptable basic salts include ammonium salts, alkali metal salts (such as sodium and potassium salts) and alkaline earth metal salts (such as magnesium and calcium salts).

Compounds having one or more chiral centers can exist in various stereoisomeric forms, i.e., each chiral center can have an R or S configuration or can be a mixture of both. Stereoisomers are compounds that differ only in their spatial arrangement. Stereoisomers include all diastereomeric and enantiomeric forms of a compound. Enantiomers are stereoisomers that are mirror images of each other. Diastereomers are stereoisomers having two or more chiral centers that are not identical and are not mirror images of each other.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the stereoisomeric purity of the named or depicted stereoisomers at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

When a disclosed compound having a chiral center is depicted by a structure without showing a configuration at that chiral center, the structure is meant to encompass the compound with the S configuration at that chiral center, the compound with the R configuration at that chiral center, or the compound with a mixture of the R and S configuration at that chiral center. When a disclosed compound having a chiral center is depicted by its chemical name without indicating a configuration at that chiral center with “S” or “R”, the name is meant to encompass the compound with the S configuration at that chiral center, the compound with the R configuration at that chiral center or the compound with a mixture of the R and S configuration at that chiral center.

When two stereoisomers are depicted by their chemical names or structures, and the names or structures are connected by an “or”, one or the other of the two stereoisomers is intended, but not both.

A racemic mixture means a mixture of 50% of one enantiomer and 50% of its corresponding enantiomer. The present teachings encompass all enantiomerically-pure, enantiomerically-enriched, diastereomerically pure, diastereomerically-enriched, and racemic mixtures, and diastereomeric mixtures of the compounds disclosed herein.

Enantiomeric and diastereomeric mixtures can be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well-known asymmetric synthetic methods.

“First eluting compound” or “Peak 1” in the Experimental section refers to an intended reaction product compound obtained from a chromatography separation/purification that elutes earlier than a second intended reaction product compound from the same preceding reaction. The second intended product compound is referred to as “Second eluting compound” or “Peak 2”. In the present disclosure, the first eluting isomer corresponds to the lower compound number in the enantiomeric pair.

In the compounds of the disclosure, any position specifically designated as “D” or “deuterium” is understood to have deuterium enrichment at 50, 80, 90, 95, 98 or 99%. “Deuterium enrichment” is a mole percent and is determined by dividing the number of compounds with deuterium at the indicated position by the total number of all of the compounds. When a position is designated as “H” or “hydrogen”, the position has hydrogen at its natural abundance. When a position is silent as to whether hydrogen or deuterium is present, the position has hydrogen at its natural abundance. One specific alternative embodiment is directed to a compound of the disclosure having deuterium enrichment of at least 5, 10, 25, 50, 80, 90, 95, 98 or 99% at one or more positions not specifically designated as “D” or “deuterium”.

As used herein, many moieties (e.g., alkyl, alkoxy, cycloalkyl or heterocyclyl) are referred to as being either “substituted” or “optionally substituted”. When a moiety is modified by one of these terms, unless otherwise noted, it denotes that any portion of the moiety that is known to one skilled in the art as being available for substitution can be substituted, which includes one or more substituents. Where if more than one substituent is present, then each substituent may be independently selected. Such means for substitution are well-known in the art and/or taught by the instant disclosure. The optional substituents can be any substituents that are suitable to attach to the moiety.

Compounds of the disclosure are selective EGFR inhibitors. As used herein, the term “selective EGFR inhibitor” means a compound which selectively inhibits certain mutant EGFR kinases over wild-type EGFR and the kinome. Said another way, a selective EGFR inhibitor has no or low activity against wild-type EGFR and the kinome. A selective EGFR inhibitor's inhibitory activity against certain mutant EGFR kinases is more potent in terms of IC₅₀ value (i.e., the IC₅₀ value is subnanomolar) when compared with its inhibitory activity against wild-type EGFR and many other kinases. Potency can be measured using known biochemical assays.

Some compounds of the disclosure have the advantage of good penetration of the brain. The ability of a particular compound to cross the BBB and penetrate the brain can be assessed using a variety of known methods or combinations of such methods. One in vitro method that is frequently used to predict a compound's in vivo brain penetration is P-gp efflux ratio. P-glycoprotein (P-gp) is expressed at the blood-brain barrier (BBB) and restricts the penetration of its substrates into the central nervous system (CNS). Compounds that are found to be good P-gp substrates in vitro (i.e., have a high efflux ratio) are predicted to have poor in vivo brain penetration. In order to measure the P-gp efflux ratio, Madin-Darby canine kidney cells overexpressing P-gp (MDCK-MDR1 cells) the apparent apical to basolateral permeability (Papp[A-B]) and the apparent basolateral to apical permeability (Papp[B-A]) for compounds is determined. The P-gp efflux ratio is a measure of the ratio of Papp[B-A]/Papp[A-B]. In some embodiments, a compound of the disclosure has a P-gp efflux ratio of less than 2, less than 3, less than 4, less than 5.

Some compounds of the disclosure have the advantage of good metabolic stability. One indicator of good metabolic stability is high microsomal stability. Hepatic metabolism is a predominant route of elimination for small molecule drugs. The clearance of compounds by hepatic metabolism can be assessed in vitro using human liver microsomes (HLMs) or human hepatocytes.

Compounds are incubated with HLMs plus appropriate co-factors or human hepatocytes and compound depletion is measured to determine an in vitro intrinsic clearance (Clint). The Clint is scaled to total body clearance (CL), and a hepatic extraction ratio (ER) is determined by dividing CL to standard human hepatic blood flow. Compounds that have a low hepatic extraction ratio are considered to have good metabolic stability. In some embodiments, a compound of the disclosure has a calculated ER of <0.3, <0.4, <0.5, <0.6.

### Pharmaceutical Compositions

Pharmaceutical compositions of the disclosure (also referred to herein as the “disclosed pharmaceutical compositions”) comprise one or more pharmaceutically acceptable carrier(s) or diluent(s) and a compound of the disclosure (e.g., a compound of Formula (I)), or a pharmaceutically acceptable salt thereof.

“Pharmaceutically acceptable carrier” and “pharmaceutically acceptable diluent” refer to a substance that aids the formulation and/or administration of an active agent to and/or absorption by a subject and can be included in the pharmaceutical compositions of the disclosure without causing a significant adverse toxicological effect on the subject. Non-limiting examples of pharmaceutically acceptable carriers and/or diluents include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, hydroxymethycellulose, fatty acid esters, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds or pharmaceutically acceptable salts thereof.

The pharmaceutical compositions of the disclosure optionally include one or more pharmaceutically acceptable carriers and/or diluents therefor, such as lactose, starch, cellulose and dextrose. Other excipients, such as flavoring agents, sweeteners, and preservatives, such as methyl, ethyl, propyl and butyl parabens, can also be included. More complete listings of suitable excipients can be found in the Handbook of Pharmaceutical Excipients (5^{th} Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003—20th edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

### Methods of Treatment

The present disclosure provides a method of inhibiting certain mutant forms of epidermal growth factor receptor (EGFR) in a subject in need thereof, comprising administering to the subject an effective amount of a compound disclosed herein, a pharmaceutically acceptable salt thereof or a pharmaceutical composition disclosed herein. Mutant forms of EGFR include for example, EGFR with LRCS mutation (the exon 19 deletion (del19) or exon 21 (L858R) substitution mutation, and C797X (e.g., C797S) mutation). Subjects “in need of inhibiting EGFR” are those having a disease for which a beneficial therapeutic effect can be achieved by inhibiting at least one mutant EGFR, e.g., a slowing in disease progression, alleviation of one or more symptoms associated with the disease or increasing the longevity of the subject in view of the disease.

In some embodiments, the disclosure provides a method of treating a disease/condition/or cancer associated with or modulated by mutant EGFR, wherein the inhibition of the mutant EGFR is of therapeutic benefit, including but not limited to the treatment of cancer in a subject in need thereof. The method comprises administering to the subject an effective amount of a compound disclosed herein, a pharmaceutically acceptable salt thereof, or pharmaceutical composition disclosed herein.

In another embodiment, the disclosure provides a method of treating a subject with cancer, comprising administering to the subject an effective amount of a compound disclosed herein, a pharmaceutically acceptable salt thereof, or a pharmaceutical composition disclosed herein. Cancers to be treated according to the disclosed methods include lung cancer, colon cancer, urothelial cancer, breast cancer, prostate cancer, brain cancers, ovarian cancer, gastric cancer, pancreatic cancer, head and neck cancer, bladder cancer, and mesothelioma, including metastasis (in particular brain metastasis) of all cancers listed. Typically, the cancer is characterized by at one or more EGFR mutations described herein. In a specific embodiment, the cancer has progressed on or after EGFR tyrosine kinase inhibitor (TKI) therapy. In a specific embodiment, the disease has progressed on or after first line 3^{rd} generation TKI, e.g. osimertinib. In a specific embodiment, the cancer was not previously treated.

In a specific embodiment, the cancer to be treated is lung cancer. In a more specific embodiment, the cancer is non-small cell lung cancer (NSCLC). In some embodiments, the lung cancer is locally advanced or metastatic NSCLC, NSCLC adenocarcinoma, NSCLC with squamous histology and NSCLC with non-squamous histology. In another embodiment, the lung cancer is NSCLC adenocarcinoma. In another specific embodiment, the lung cancer (or non-small cell lung cancer) has metastasized to the brain.

In another embodiment, the disease/condition/or cancer associated with or modulated by mutant EGFR that is characterized by an EGFR genotype selected from genotypes 1-36 according Table 2 below (del18=Exon 18 deletion, specifically, e.g., del E709_T710 insD; and del19=Exon 19 deletion, specifically, e.g., delE746_A750 (most common), delE746_S752insV, del747_A750insP, delL747_P753insS, and delS752_I759; ex20ins—Exon 20 insertion, specifically, e.g., D761-E762insX, A763-Y764insX, Y764-V765insX, V765-M766insX, A767-S768insX, S768-D769insX, V769-D770insX, N771-P772insX, P772-H773insX, H773-V774insX, and V774-C775insX):

TABLE 2
EGFR Genotype

| | |
|---|---|
| 1 | EGFR del19 |
| 2 | EGFR del19 C797S |
| 3 | EGFR del19 C797X (C797G or C797N or C797Y or |
| | C797T or C797D) |
| 4 | EGFR del19 L792X (L792F, L792H or L792Y) |
| 5 | EGFR del19 G796R (G796S) |
| 6 | EGFR del19 L792R (L792V or L792P) |
| 7 | EGFR del19 L718Q (L718V) |
| 8 | EGFR del19 G724S |
| 9 | EGFR del19 S768I (SV768IL) |
| 10 | EGFR del 19 V834L |
| 11 | EGFR del19 C797S L718Q (L718V) |
| 12 | EGFR del19 L718Q (L718V) A750P |
| 13 | EGFR L858R |
| 14 | EGFR L858R C797S |
| 15 | EGFR L858R C797X (797G or C797N or C797Y or |
| | C797T or C797D) |
| 16 | EGFR L858R L792X (L792F, L792H or L792Y) |
| 17 | EGFR L858R G796R (G796S) |
| 18 | EGFR L858R L792R (L792V or L792P) |
| 19 | EGFR L858R L718Q (L718V) |
| 20 | EGFR L858R G724S |
| 21 | EGFR L858R S768I (SV768IL) |
| 22 | EGFR L858R V834L |
| 23 | EGFR L858R C797S L718Q (L718V) |
| 24 | EGFR L858R L718Q (L718V) A750P |
| 25 | EGFR L861Q |
| 27 | EGFR L861Q C797S/G/N |
| 28 | EGFR del18 |
| 29 | EGFR G719X (G719A, G719S, G719C, G719R, |
| | G719D, or G719V) |
| 30 | EGFR E709X (E709K, E709H, or E709A) |
| 31 | EGFR E709X (E709K, E709H, or E709A) (G719A, |
| | G719S, G719C, G719D, G719R, or G719V) |
| 32 | EGFR G719X (G719A, G719S, G719C, G719D, |
| | G719R, or G719V) S768I |
| 33 | EGFR S768I |
| 34 | EGFR ex20ins |
| 35 | EGFR ex20ins L718Q |
| 36 | EGFR ex20ins C797S |

In another embodiment, the disease/condition/or cancer (e.g., NSCLC) being treated with a disclosed compound, a pharmaceutically acceptable salt or a pharmaceutical composition disclosed herein is characterized by EGFR comprising EGFR del19.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR del19 C797S.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR del9 C797X (C797G or C797N or C797Y or C797T or C797D).

In another embodiment, the disease/condition/or cancer (e.g., NSCLC) being treated with a disclosed compound, a pharmaceutically acceptable salt, or a pharmaceutical composition disclosed herein is characterized by EGFR comprising EGFR del9 L792X (L792F, L792H or L792Y).

In another embodiment, the disease/condition/or cancer (e.g., NSCLC) being treated with a disclosed compound, a pharmaceutically acceptable salt thereof, or pharmaceutical composition disclosed herein is characterized by EGFR comprising EGFR del19 G796R (G796S).

In another embodiment, the disease/condition/or cancer (e.g., NSCLC) being treated with a disclosed compound, a pharmaceutically acceptable salt thereof, or pharmaceutical composition disclosed herein is characterized by EGFR comprising EGFR del19 L792R (L792V or L792P).

In another embodiment, the disease/condition/or cancer (e.g., NSCLC) being treated with a disclosed compound, a pharmaceutically acceptable salt thereof, or pharmaceutical composition disclosed herein is characterized by EGFR comprising EGFR del19 L718Q (L718V).

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R.

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R C797S.

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R C797X (797G or C797N or C797Y or C797T or C797D).

In another embodiment, the disease/condition/or cancer (e.g., NSCLC) being treated with a disclosed compound, a pharmaceutically acceptable salt thereof or a pharmaceutical composition disclosed herein is characterized by EGFR comprising EGFR L858R L792X (L792F, L792H or L792Y).

In another embodiment, the disease/condition/or cancer (e.g., NSCLC) being treated with a disclosed compound, a pharmaceutically acceptable salt thereof or pharmaceutical composition disclosed herein is characterized by EGFR comprising EGFR L858R G796R (G796S).

In another embodiment, the disease/condition/or cancer (e.g., NSCLC) being treated with a disclosed compound, a pharmaceutically acceptable salt thereof or pharmaceutical composition disclosed herein is characterized by EGFR comprising EGFR L858R L792R (L792V or L792P).

In another embodiment, the disease/condition/or cancer (e.g., NSCLC) being treated with a disclosed compound, a pharmaceutically acceptable salt thereof or pharmaceutical composition disclosed herein is characterized by EGFR comprising EGFR L858R L718Q (L718V).

In another embodiment, the disease/condition/or cancer (e.g., NSCLC) being treated with a disclosed compound, a pharmaceutically acceptable salt or a pharmaceutical composition disclosed herein is characterized by EGFR comprising EGFR del8.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR G719X (G719A, G719S, G719C, G719R, G719D, or G719V).

In another embodiment, the disease/condition/or cancer (e.g., NSCLC) being treated with a disclosed compound, a pharmaceutically acceptable salt or a pharmaceutical composition disclosed herein is characterized by EGFR comprising EGFR E709X (E709K, E709H, or E709A).

In another embodiment, the disease/condition/or cancer (e.g., NSCLC) being treated with a disclosed compound, a pharmaceutically acceptable salt or a pharmaceutical composition disclosed herein is characterized by EGFR comprising EGFR E709X (E709K, E709H, or E709A) (G719A, G719S, G719C, G719D, G719R, or G719V).

In another embodiment, the disease/condition/or cancer (e.g., NSCLC) being treated with a disclosed compound, a pharmaceutically acceptable salt or a pharmaceutical composition disclosed herein is characterized by EGFR comprising EGFR G719X (G719A, G719S, G719C, G719D, G719R, or G719V) S768I.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR S768I.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR ex20ins.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR ex20ins L718Q.

In another embodiment, the disease/condition/or cancer (e.g., NSCLC) being treated with a disclosed compound, a pharmaceutically acceptable salt or a pharmaceutical composition disclosed herein is characterized by EGFR comprising EGFR ex20ins C797S.

In another embodiment, the disease/condition/or cancer (e.g., NSCLC) being treated with a disclosed compound, a pharmaceutically acceptable salt or a pharmaceutical composition disclosed herein is characterized by an EGFR genotype selected from genotypes 1-36.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to osimertinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to afatinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to dacomitinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to lazertinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to osimertinib and afatinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to osimertinib and dacomitinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to amivantamab.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to amivantamab and lazertinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to aumolertinib (formerly almonertinib).

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to olmutinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to nazartinib.

In another embodiment, the disease/condition/or cancer (e.g., NSCLC) being treated with a disclosed compound, a pharmaceutically acceptable salt or a pharmaceutical composition disclosed herein is characterized by EGFR mutations that confer resistance to avitinib.

Another embodiment is the treatment a subject with metastatic NSCLC with tumors harboring activating Exon 19 Deletion or L858R EGFR mutations, G719X (A, S, C, D, R, V), S768I and L861Q, as well as a resistance mutation disclosed herein as detected by an approved molecular testing methodology.

Another embodiment is a disclosed compound used in combination with a 2^{nd} or 3^{rd} generation TKI indicated for the treatment of subject with metastatic NSCLC with tumors harboring C797X mutations as detected by an approved test, and whose disease has progressed on or after 1 or 2 prior EGFR TKI therapies.

Another embodiment is a disclosed compound for the treatment of subjects with metastatic NSCLC whose disease with on-target EGFR resistance has progressed on or after any EGFR TKI. In a specific embodiment, the disclosed compound is used in combination with a 2^{nd} or 3^{rd} generation TKI indicated for the treatment of subject with metastatic NSCLC.

Another embodiment is a disclosed compound for the treatment of subjects with metastatic EGFR C797X mutation-positive NSCLC as detected by an approved molecular test, whose disease has progressed on or after first-line or second-line osimertinib. In a specific embodiment, the disclosed compound is used in combination with a 2^{nd} or 3^{rd} generation TKI indicated for the treatment of subject with metastatic NSCLC.

In a particular embodiment, the deletions, mutations, and insertions disclosed herein are detected by an FDA-approved test.

A person of ordinary skill in the art can readily determine the certain EGFR alterations a subject possesses in a cell, cancer, gene, or gene product, e.g., whether a subject has one or more of the mutations or deletions described herein using a detection method selected from those known in the art such as hybridization-based methods, amplification-based methods, microarray analysis, flow cytometry analysis, DNA sequencing, next-generation sequencing (NGS), primer extension, PCR, in situ hybridization, fluorescent in situ hybridization, dot blot, and Southern blot.

To detect one or more EGFR deletions and/or mutations, a primary tumor sample, circulating tumor DNA (ctDNA), circulating tumor cells (CTC), and/or circulating exosomes may be collected from a subject. The samples are processed, the nucleic acids are isolated using techniques known in the art, then the nucleic acids are sequenced using methods known in the art. Sequences are then mapped to individual exons, and measures of transcriptional expression (such as RPKM, or reads per kilobase per million reads mapped), are quantified. Raw sequences and exon array data are available from sources such as TCGA, ICGC, and the NCBI Gene Expression Omnibus (GEO). For a given sample, individual exon coordinates are annotated with gene identifier information, and exons belonging to kinase domains are flagged. The exon levels are then z-score normalized across all tumors samples.

The compounds of the disclosure, pharmaceutically acceptable salts thereof or pharmaceutical compositions disclosed herein may be used for treating to a subject who has become refractory to treatment with one or more other EGFR inhibitors. “Refractory” means that the subject's cancer previously responded to drugs but later responds poorly or not at all. In some embodiments, the subject has become refractory to one or more first generation EGFR inhibitors such as erlotinib, gefitinib, icotinib or lapatinib. In some embodiments, the subject has been become refractory to treatment with one or more second generation EGFR inhibitors such as afatinib, dacomitinib, poziotinib, or neratinib. In some embodiments the subject has become refractory to treatment with one or more first generation inhibitors and one or more second generation inhibitors.

In some embodiments, the subject has become refractory to treatment with one or more third generation inhibitors such as osimertinib, nazartinib, or avitinib. In one embodiment, the subject has become refractory to treatment with one or more first generation EGFR inhibitors and one or more third generation EGFR inhibitors. In some embodiments, the subject has become refractory to treatment with one or more second generation EGFR inhibitors and one or more third generation EGFR inhibitors. In some embodiments, the subject has become refractory to treatment with one or more first generation inhibitors, and one or more third generation EGFR inhibitors.

### Combinations

The compounds of the disclosure, pharmaceutically acceptable salts thereof, or pharmaceutical compositions disclosed herein can be used in combination with one or more additional pharmacologically active substances. For example, the disclosure includes methods of treating a condition/disease/ or cancer comprising administering to a subject in need thereof a compound of the disclosure or a pharmaceutically acceptable salt or a pharmaceutical composition disclosed herein thereof in combination with an EGFR (or EGFR mutant) inhibitor, such as afatinib, osimertinib, lapatinib, erlotinib, dacomitinib, poziotinib, neratinib, gefitinib JBJ-04-125-02, alfiutinib (AST 2818), aumolertinib (formerly almonertinib) (HS10296), BBT-176, BI-4020, BPI-361175, BPI-D0316, CH7233163, gilitertinib, icotinib, JND-3229, lazertinib, nazartinib (EGF 816), avitinib, PCC-0208027, rezivertinib (BPI-7711), TQB3804, zorifertinib (AZ-3759), or DZD9008; an EGFR antibody such as cetuximab, panitumumab, necitumumab, HLX07, JMT101; or a bispecific EGFR and MET antibody (e.g., amivantamab ((JNJ-61186372, JNJ-372)). For the treatment of cancer e.g., NSCLC using a compound of the disclosure or pharmaceutically acceptable salt thereof or pharmaceutical composition disclosed herein in combination with a first line therapy, for example a first, second, or third generation EGFR inhibitor (i.e., as an initial treatment before the cancer has become refractory) may forestall or delay the cancer from becoming refractory. Typically, the cancer is characterized by one of the EGFR genotypes described herein.

In one aspect, a compound of the disclosure, a pharmaceutically acceptable salt thereof or a pharmaceutical composition disclosed herein can be administered in combination with a compound disclosed in International Application Publication No. WO 2021/133809, a pharmaceutically acceptable salt thereof or a pharmaceutical composition comprising the same.

In one embodiment, a compound of the disclosure, a pharmaceutically acceptable salt thereof or a pharmaceutical composition disclosed herein can be administered in combination with a compound provided below,
(3S,4R)-3-fluoro-1-(4-(5-isopropyl-8-((2R,3S)-2-methyl-3-(methylsulfonylmethyl)azetidin-1-yl)isoquinolin-3-ylamino)pyrimidin-2-yl)-4-methylpiperidin-4-ol,
(3R,4S)-3-fluoro-1-(4-(5-isopropyl-8-((2R,3S)-2-methyl-3-(methylsulfonylmethyl)azetidin-1-yl)isoquinolin-3-ylamino)pyrimidin-2-yl)-4-methylpiperidin-4-ol,
N-(2-((3S,4R)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine,
N-(2-((3R,4S)-3-fluoro-4-methoxypiperidin-1-yl)pyrimidin-4-yl)-5-isopropyl-8-((2R,3S)-2-methyl-3-((methylsulfonyl)methyl)azetidin-1-yl)isoquinolin-3-amine,
N-(2-((3S,4R)-3-fluoro-4-(methoxy-d3)piperidin-1-yl)pyrimidin-4-yl)-5-isopropyl-8-((2R,3S)-2-methyl-3-((methylsulfonyl)methyl)azetidin-1-yl)isoquinolin-3-amine,
N-(2-((3R,4S)-3-fluoro-4-(methoxy-d3)piperidin-1-yl)pyrimidin-4-yl)-5-isopropyl-8-((2R,3S)-2-methyl-3-((methylsulfonyl)methyl)azetidin-1-yl)isoquinolin-3-amine,
(3S,4R)-1-(4-(8-((2R,3S)-3-(ethylsulfonylmethyl)-2-methylazetidin-1-yl)-5-isopropyl-2,6-naphthyridin-3-ylamino)pyrimidin-2-yl)-3-fluoro-4-methylpiperidin-4-ol,
(3S,4R)-1-(4-(8-((2R,3S)-3-(ethylsulfonylmethyl)-2-methylazetidin-1-yl)-5-isopropyl-2,7-naphthyridin-3-ylamino)pyrimidin-2-yl)-3-fluoro-4-methylpiperidin-4-ol,
2-((3S,4R)-1-(4-(8-((2R,3S)-3-(ethylsulfonylmethyl)-2-methylazetidin-1-yl)-5-isopropylisoquinolin-3-ylamino)pyrimidin-2-yl)-3-fluoropiperidin-4-yloxy)ethanol,
(3S,4S)-5,5-difluoro-1-(4-(5-isopropyl-8-((2R,3S)-2-methyl-3-(methylsulfonylmethyl)azetidin-1-yl)isoquinolin-3-ylamino)pyrimidin-2-yl)-4-methoxypiperidin-3-ol,
(3R,4R)-5,5-difluoro-1-(4-(5-isopropyl-8-((2R,3S)-2-methyl-3-(methylsulfonylmethyl)azetidin-1-yl)isoquinolin-3-ylamino)pyrimidin-2-yl)-4-methoxypiperidin-3-ol,
(3S,4S)-1-(4-(8-((2R,3S)-3-(ethylsulfonylmethyl)-2-methylazetidin-1-yl)-5-isopropylisoquinolin-3-ylamino)pyrimidin-2-yl)-4-methoxypiperidin-3-ol,
(3R,4R)-1-(4-(8-((2R,3S)-3-(ethylsulfonylmethyl)-2-methylazetidin-1-yl)-5-isopropylisoquinolin-3-ylamino)pyrimidin-2-yl)-4-methoxypiperidin-3-ol,
   a pharmaceutically acceptable salt thereof, or a pharmaceutical composition comprising the same.

Alternatively, a compound of the disclosure, a pharmaceutically acceptable salt thereof or a pharmaceutical composition disclosed herein can be administered in combination with other anti-cancer agents that are not EGFR inhibitors e.g., in combination with MEK, including mutant MEK inhibitors (trametinib, cobimtetinib, binimetinib, selumetinib, refametinib); c-MET, including mutant c-Met inhibitors (savolitinib, cabozantinib, foretinib, glumetinib, tepotinib) and MET antibodies (emibetuzumab, telisotuzumab vedotin (ABBV 339)); mitotic kinase inhibitors (CDK4/6 inhibitors such as palbociclib, ribociclib, abemacicilb, GIT38); anti-angiogenic agents e.g., bevacizumab, nintedanib; apoptosis inducers such as Bcl-2 inhibitors e.g, venetoclax, obatoclax, navitoclax, palcitoclax (APG-1252), and Mcl-1 inhibitors e.g., AZD-5991, AMG-176, S-64315; mTOR inhibitors e.g, rapamycin, temsirolimus, everolimus, ridoforolimus; RET inhibitors, like pralsetinib and selpercatinib, and PI3K inhibitors dactolisib (BEZ235), pictilisib (GDC-0941), LY294002, idelalisib (CAL-101); JAK inhibitors (e.g., AZD4205, itacitinib), Aurora A inhibitors (e.g., alisertib); BCR/ABL and/or Src family tyrosine kinase inhibitors (e.g., dasatinib); VEGF inhibitors (e.g., MP0250; ramucirumab); multi-kinase protein inhibitors (e.g., anlotinib, midostaurin); PARP inhibitors (e.g., niraparib); platinum therapies (e.g., cisplatin (CDDP), carboplatin (CBDCA), or nedaplatin (CDGP)); PD-L1 inhibitors (e.g., durvalumab (MEDI 4736)); HER2/neu receptor inhibitors (e.g., trastuzumab); anti-HER2 or anti-HER3 antibody-drug conjugates (e.g., patritumab deruxtecan (U3-1402), trastuzumab emtansine); or immunogene therapy (e.g., oncoprex).

A “subject” is a human in need of treatment.

### Methods of Administration and Dosage Forms

The precise amount of compound administered to provide an “effective amount” to the subject will depend on the mode of administration, the type, and severity of the cancer, and on the characteristics of the subject, such as general health, age, sex, body weight, and tolerance to drugs. The skilled artisan will be able to determine appropriate dosages depending on these and other factors. When administered in combination with other therapeutic agents, e.g., when administered in combination with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of Formula (I) being used by following, for example, dosages reported in the literature and recommended in the Physician's Desk Reference (57th Ed., 2003).

“Treating” or “treatment” refers to obtaining a desired pharmacological and/or physiological effect. The effect can be therapeutic, which includes achieving, partially or substantially, one or more of the following results: partially or substantially reducing the extent of the disease, condition or cancer; ameliorating or improving a clinical symptom or indicator associated with the disease, condition or cancer; delaying, inhibiting or decreasing the likelihood of the progression of the disease, condition or cancer; or decreasing the likelihood of recurrence of the disease, condition or cancer.

The term “effective amount” means an amount when administered to the subject which results in beneficial or desired results, including clinical results, e.g., inhibits, suppresses or reduces the symptoms of the condition being treated in the subject as compared to a control. For example, a therapeutically effective amount can be given in unit dosage form (e.g., 0.1 mg to about 50 g per day, alternatively from 1 mg to about 5 grams per day; and in another alternatively from 10 mg to 1 gram per day).

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

In addition, a compound of the disclosure, a pharmaceutically acceptable salt thereof or a pharmaceutical composition of the disclosure can be co-administered with other therapeutic agents. As used herein, the terms “co-administration”, “administered in combination with”, and their grammatical equivalents, are meant to encompass administration of two or more therapeutic agents to a single subject, and are intended to include treatment regimens in which the agents are administered by the same or different route of administration or at the same or different times. In some embodiments the one or more compounds of the disclosure, a pharmaceutically acceptable salt thereof or a pharmaceutical composition of the disclosure will be co-administered with other agents. These terms encompass administration of two or more agents to the subject so that both agents and/or their metabolites are present in the subject at the same time. They include simultaneous administration in separate compositions, administration at different times in separate compositions, and/or administration in a composition in which both agents are present. Thus, in some embodiments, the compounds described herein and the other agent(s) are administered in a single composition. In some embodiments, the compounds described herein and the other agent(s) are admixed in the composition.

The particular mode of administration and the dosage regimen will be selected by the attending clinician, taking into account the particulars of the case (e.g. the subject, the disease, the disease state involved, the particular treatment). Treatment can involve daily or multi-daily or less than daily (such as weekly or monthly etc.) doses over a period of a few days to months, or even years. However, a person of ordinary skill in the art would immediately recognize appropriate and/or equivalent doses looking at dosages of approved compositions for treating a disease using the disclosed EGFR inhibitors for guidance.

The compounds of the disclosure or a pharmaceutically acceptable salt thereof can be administered to a patient in a variety of forms depending on the selected route of administration, as will be understood by those skilled in the art. The compounds of the present teachings may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time.

The pharmaceutical composition of the disclosure is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In preferred embodiments, the pharmaceutical composition is formulated for intravenous administration.

Typically, for oral therapeutic administration, a compound of the disclosure or a pharmaceutically acceptable salt thereof may be incorporated with excipient and used in the form of ingestible tablets, buccal tablets, troches, capsules, elixirs, suspensions, syrups, wafers, and the like.

Typically for parenteral administration, solutions of a compound of the disclosure can generally or a pharmaceutically acceptable salt thereof be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms.

Typically, for injectable use, sterile aqueous solutions or dispersion of, and sterile powders of, a compound of the disclosure for the extemporaneous preparation of sterile injectable solutions or dispersions are appropriate.

The following examples are intended to be illustrative and are not intended to be limiting in any way to the scope of the disclosure.

### EXEMPLIFICATION

### Preparation of Exemplary Compounds

Definitions

| | |
|---|---|
| ACN | acetonitrile |
| AcOH | acetic Acid |
| C, ° C. | degrees Celsius |
| CMPI | 2-Chloro-1-methylpyridinium iodide |
| DCM | dichloromethane |
| DIPEA | diisopropylethylamine |
| DMF | dimethyl formamide |
| DMF-DMA | N,N-Dimethylformamide dimethyl acetal |
| DMSO | dimethylsulfoxide |
| DBAD | Di-tert-butyl azodicarboxylate |
| DMAP | Dimethylaminopyridine |
| DBU | 1,8-Diazabicyclo[5.4.0]undec-7-ene |
| EA | ethyl acetate |
| H, h, hr(s) | hour(s) |
| HPLC | high performance liquid chromatography |
| IC50 | inhibitory concentration 50% |
| Int | Intermediate |
| IPA, i-PrOH | isopropyl alcohol |
| min | minutes |
| MTBE | methyl tert-butyl ether |
| MeOH | methanol |
| MW | microwave |
| PE | petroleum ether |
| rt | room temperature |
| TEA | triethylamine |
| THF | tetrahydrofuran |
| RT | retention time |
| Prep HPLC | preparative high-performance liquid chromatography |
| Prep-TLC | preparative thin layer chromatography |
| TFA | trifluoracetic acid |
| TLC | thin layer chromatography |
| MsCl | methanesulfonyl chloride |
| TBS | tert-butyldimethylsilyl |
| Bpin | boronic acid pinacol ester |
| BINAP | (2,2′-bis(diphenylphosphino)-1,1′-binaphthyl) |
| Pd(dppf)Cl2 | [1,1′- |
| | Bis(diphenylphosphino)ferrocene]dichloropalladium |
| PPh₃ | triphenylphosphine |
| (Boc)2O | Di-tert-butyl dicarbonate |
| KHMDS | Potassium hexamethyldisilazide |
| HATU | (1-[Bis(dimethylamino)methylene]-1H-1,2,3- |
| | triazolo[4,5-b]pyridinium 3-oxide hexafluorophosphate |

Methods for preparing compounds of the invention can be carried out in suitable solvents which can be readily selected by one of skill in the art of organic synthesis. Suitable solvents can be substantially non-reactive with the starting materials (reactants), intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in Wuts and Greene, Protective Groups in Organic Synthesis, 5th ed., John Wiley & Sons: New Jersey, (2014), which is incorporated herein by reference in its entirety.

Reactions can be monitored according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC). Analytical instruments and methods for compound characterization: LC-MS: Unless otherwise indicated, all liquid chromatography-mass spectrometry (LC-MS) data (sample analyzed for purity and identity) were obtained with an Agilent model-1260 LC system using an Agilent model 6120 mass spectrometer utilizing ES-API ionization fitted with an Agilent Poroshel 120 (EC-C18, 2.7 um particle size, 3.0×50 mm dimensions) reverse-phase column at 22.4 degrees Celsius. The mobile phase consisted of a mixture of solvent 0.1% formic acid in water and 0.1% formic acid in acetonitrile. A constant gradient from 95% aqueous/5% organic to 5% aqueous/95% organic mobile phase over the course of 4 minutes was utilized. The flow rate was constant at 1 mL/min.

Prep LC-MS: Preparative HPLC was performed on a Shimadzu Discovery VP® Preparative system fitted with a Luna 5 u C18(2) 100A, AXIA packed, 250×21.2 mm reverse-phase column at 22.4 degrees Celsius. The mobile phase consisted of a mixture of solvent 0.1% formic acid in water and 0.1% formic acid in acetonitrile. A constant gradient from 95% aqueous/5% organic to 5% aqueous/95% organic mobile phase over the course of 25 minutes was utilized. The flow rate was constant at 20 mL/min. Reactions carried out in a microwave were done so in a Biotage Initiator microwave unit.

Silica gel chromatography: Silica gel chromatography was performed on either a Teledyne Isco CombiFlash® Rf unit or a Biotage® Isolera Four unit.

Proton NMR Unless otherwise indicated, all ¹H NMR spectra were obtained with a Varian 400 MHz Unity Inova 400 MHz NMR instrument (acquisition time=3.5 seconds with a 1 second delay; 16 to 64 scans). Where characterized, all protons were reported in DMSO-d6 solvent as parts-per million (ppm) with respect to residual DMSO (2.50 ppm).

### Preparation of Intermediates

### Intermediate BP-1: 3-methoxy-1-methyl-4-(4,4,5,5-tetramethyl-1,3,2-dioxaborolan-2-yl)-1H-pyrazole

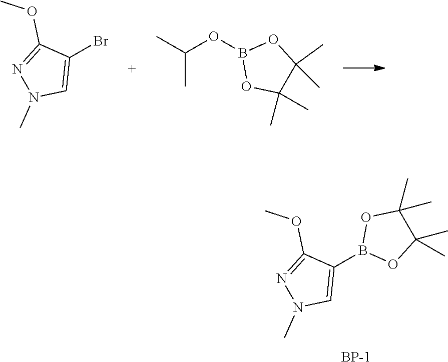

In a 50 mL flask, was added 4-bromo-3-methoxy-1-methyl-1H-pyrazole (600 mg, 3.14 mmol) dissolved in THF (10 mL), cooled with liquid N2 to −78° C. To this was added n-BuLi (2.5 M, 1.5 mL) dropwise. The mixture was stirred for 30 mins. 4,4,5,5-tetramethyl-2-(propan-2-yloxy)-1,3,2-dioxaborolane (699 mg, 3.76 mmol) was added to the reaction and stirred at room temperature for 3 hrs. The reaction was diluted with water and ethyl acetate. The organic layers were combined and further purified by prep-TLC (PE/EA=1/3) to afford the title compound as a white solid (300 mg). LC-MS: (ES, m/z): RT=1.179 min, LCMS: m/z=239 [M+1].

### Intermediate BP-3: N-(7-bromo-5-((3,3-difluoro-1-methylpiperidin-4-yl)oxy)quinazolin-4-yl)benzo[d]thiazol-6-amine

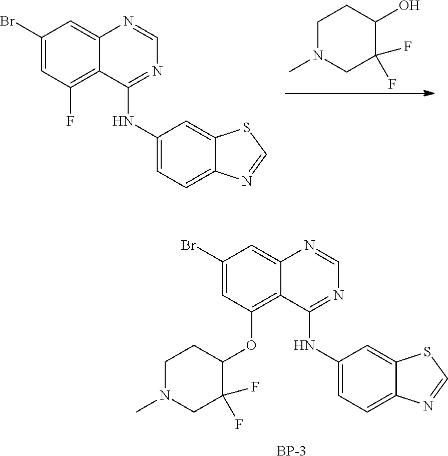

To a solution of (R)-3,3-difluoro-1-methylpiperidin-4-ol (332 mg, 2.2 mmol) and Intermediate BP-59 in DMF (10 mL) was added NaH (88 mg, 2.2 mmol). The mixture was stirred at 25° C. for 10 min. N-(7-bromo-5-fluoroquinazolin-4-yl)benzo[d]thiazol-6-amine (550 mg, 1.46 mmol) was added and the reaction stirred at 80° C. for 2 hrs. The reaction was quenched with water and concentrated to dryness. The residue was purified by prep-TLC (DCM:MeOH=5:1) to afford N-(7-bromo-5-(3,3-difluoro-1-methylpiperidin-4-yloxy)quinazolin-4-yl)benzo[d]thiazol-6-amine (500 mg, yield: 67%) as an off-white solid. LC-MS: (ES, m/z): RT=1.240 min, LCMS: m/z=506,508 [M+1]

### Intermediate BP-4: (4-methoxy-1-methyl-1H-pyrazol-3-yl)boronic acid

### Intermediate BP-4a: 3-bromo-4-methoxy-1-methyl-1H-pyrazole

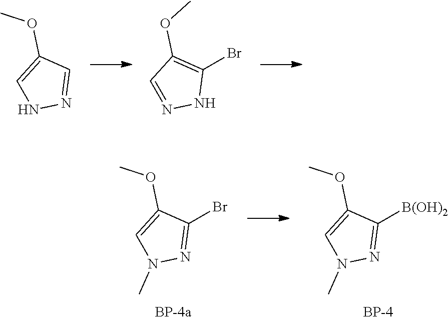

### Step 1: Synthesis of 3-bromo-4-methoxy-1H-pyrazole

To a solution of 4-methoxy-1H-pyrazole (1 g, 10.1 mmol) in DCM (25 mL) was added NBS (1.88 g, 10.6 mmol) portion-wise. The mixture was stirred at 25° C. for 1 hr. The solution was concentrated to dryness and the residue purified by silica gel column chromatography with 25% EA in PE to afford the title compound (1.6 g, yield: 89%) as a white solid. LC-MS: (ES, m/z): RT=0.675 min, LCMS: m/z=177, 179 [M+1]

### Step 2: Synthesis of 3-bromo-4-methoxy-1-methyl-1H-pyrazole

To a solution of 3-bromo-4-methoxy-1H-pyrazole (1.6 g, 9.03 mmol) in THF (30 mL) was added NaH (431 mg, 10.8 mmol). The mixture was stirred at 25° C. for 10 min. Mel (1.53 g, 10.8 mmol) was added and the reaction stirred for 1 hour. The reaction was quenched with ice-water and concentrated to dryness. The residue was purified by prep-TLC with EA:PE=1:4 to afford the title compound (1.2 g, yield: 69%) as a light-yellow oil. LC-MS: (ES, m/z): RT=0.977 min, LCMS: m/z=191, 193 [M+1]

### Step 3: Synthesis of (4-methoxy-1-methyl-1H-pyrazol-3-yl)boronic acid

BuLi (2.5 M, 2 mL, 5.22 mmol) was added to a solution of 3-bromo-4-methoxy-1-methyl-1H-pyrazole (500 mg, 2.61 mmol) and B(iPrO)₃ (1.47 g, 7.83 mmol) in THF (15 mL) at −70° C. The temperature was raised to −10° C. for 1 hour. The reaction was quenched with water, concentrated to dryness to afford the crude title compound (1.5 g, purity: 20%, yield: 73%) as a white semi-solid. LC-MS: (ES, m/z): RT=0.475 min, 0.508 min, LCMS: m/z=157 [M+1].

### Intermediate BP-5: 1-ethyl-3-hydroxypyrrolidin-2-one

### Intermediate BP-5a: (rac)-3-((tert-butyldimethylsilyl)oxy)pyrrolidin-2-one

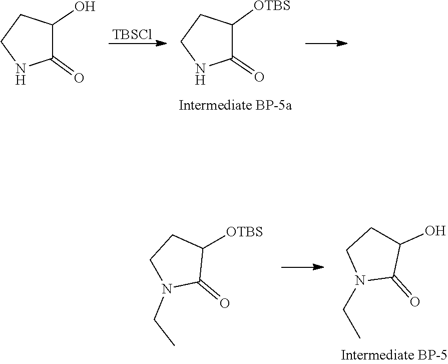

### Step 1: Intermediate BP-5a: (rac)-3-((tert-butyldimethylsilyl)oxy)pyrrolidin-2-one

To a solution of (rac)-3-hydroxypyrrolidin-2-one (1.0 g, 9.89 mmol) in CH₂Cl₂ (39.6 mL) was added DMAP (0.036 g, 0.297 mmol), imidazole (1.347 g, 19.78 mmol) and tert-butylchlorodimethylsilane (1.789 g, 11.87 mmol). The resulting mixture was stirred at rt for 16 h and then washed with water (2×50 mL) and brine (50 mL), dried over anhydrous sodium sulfate, filtered and concentrated under reduced pressure. The residue was purified by silica gel chromatography (0-100% EtOAc in Hexanes) to give the title compound (1.97 g, 9.15 mmol, 93% yield) as a white solid. LC-MS: (ES, m/z): RT=2.905 min, LC-MS: m/z=216 [M+1].

### Step 2: (rac) 3-((tert-butyldimethylsilyl)oxy)-1-ethylpyrrolidin-2-one

A vial containing a mixture of (rac)-3-((tert-butyldimethylsilyl)oxy)pyrrolidin-2-one (750 mg, 3.48 mmol), cesium carbonate (4539 mg, 13.93 mmol) and iodoethane (1680 μl, 20.89 mmol) in acetonitrile (34.8 mL) was heated at 70° C. for 24 h. The reaction mixture was concentrated under reduced pressure. The residue was taken up in EtOAc (25 mL) and washed with water (2×25 mL). The organic layer was dried over anhydrous sodium sulfate, filtered and the solvent was removed under reduced pressure. The residue was purified by silica gel chromatography (0-70% EtOAc in Hexanes) to afford the title compound (559 mg, 2.30 mmol, 66% yield) as a colorless oil. LC-MS: (ES, m/z): RT=3.367 min, LC-MS: m/z=244 [M+1].

### Step 3: Intermediate BP-5: (rac)-1-ethyl-3-hydroxypyrrolidin-2-one, HCl

To a mixture of (rac)-3-((tert-butyldimethylsilyl)oxy)-1-ethylpyrrolidin-2-one (559 mg, 2.296 mmol) in CH₂Cl₂ (2.3 mL) was added HCl (4M in 1,4-dioxane, 1148 μl, 4.59 mmol). The resulting mixture was stirred at RT for 2 h and then solvent removed under reduced pressure to afford the title compound (290 mg, 1.75 mmol, 76% yield) as a viscous colorless oil Crude material was carried forward without purification. LC-MS: (ES, m/z): RT=0.667 min, LC-MS: m/z=130 [M+1]. ¹H NMR (500 MHz, CDCl₃) δ 5.83 (qd, J=13.5, 8.7, 6.9 Hz, 1H), 4.53 (t, J=8.4 Hz, 1H), 3.47-3.30 (m, 4H), 2.52-2.42 (m, 1H), 2.01 (dq, J=12.8, 8.6 Hz, 1H), 1.16 (t, J=7.3 Hz, 3H),

### Intermediate BP-6: 3-hydroxy-1-isopropylpyrrolidin-2-one

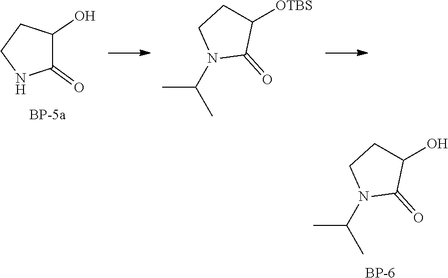

### Step 1: (rac)-3-((tert-butyldimethylsilyl)oxy)-1-isopropylpyrrolidin-2-one

A vial containing a mixture of Intermediate BP-5a (rac)-3-((tert-butyldimethylsilyl)oxy)pyrrolidin-2-one (750 mg, 3.48 mmol), cesium carbonate (4539 mg, 13.93 mmol) and iodopropane (2086 μl, 20.89 mmol) in acetonitrile (34.8 mL) was heated at 70° C. for 24 h. The reaction mixture was concentrated under reduced pressure. The residue was dissolved in EtOAc (25 mL) and washed with water (2×25 mL). The organic layer was dried over anhydrous sodium sulfate, filtered and solvent removed under reduced pressure. The residue was purified by silica gel chromatography (0-70% EtOAc in Hexanes) to afford the title compound (226 mg, 0.88 mmol, 25% yield) as a colorless oil. LC-MS: (ES, m/z): RT=4.056 min, LC-MS: m/z=258 [M+1].

### Step 2: (rac)-3-hydroxy-1-isopropylpyrrolidin-2-one, HCl

To a mixture of (rac)-3-((tert-butyldimethylsilyl)oxy)-1-isopropylpyrrolidin-2-one (226 mg, 0.88 mmol) in CH₂Cl₂ (0.88 mL) was added HCl (4M in 1,4-dioxane, 439 μl, 1.76 mmol). The resulting mixture was stirred at RT for 2 h and then solvent removed under reduced pressure to afford the title compound (119 mg, 0.66 mmol, 75% yield) as a white solid. Crude material was carried forward without purification. LC-MS: (ES, m/z): RT=1.426 min, LC-MS: m/z=144 [M+1].

### Intermediate BP-8

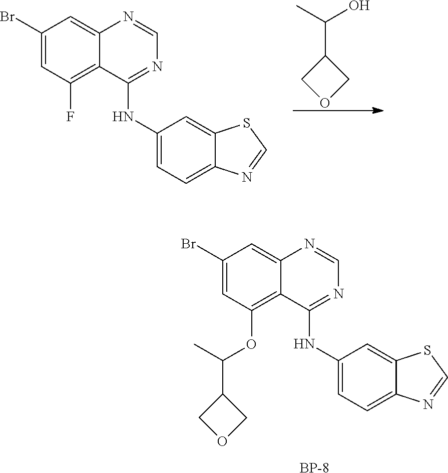

To a 20-mL sealed tube was added Intermediate BP-59 (300 mg, 799 μmol) in THF (3 mL), 1-(oxetan-3-yl)ethan-1-ol (81.6 mg, 799 μmol), potassium tert-butoxide (178 mg, 1.59 mmol). The resulting solution was stirred at 80° C. for 2 h. The resulting solution was concentrated under vacuum. The residue was purified by Prep-TLC with DCM/MeOH (20:1). This resulted in the title compound (300 mg, 82.2%) as an off-white solid. LC-MS: (ES, m/z): RT=1.102 min, LCMS: m/z=457,459 [M+1]

### Intermediate BP-9

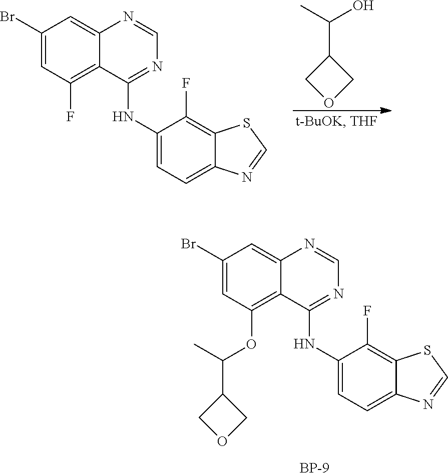

t-BuOK (85.3 mg, 762 μmol) was added to Intermediate BP-60 (150 mg, 381 μmol) and 1-(oxetan-3-yl)ethan-1-ol (155 mg, 1.52 mmol) in THF at rt. The reaction was stirred at 80 degrees for 6 h.

The mixture was diluted with EA 100 mL and washed with brine 50 mL 2×. The organic layer was dried with Na₂SO₄ and concentrated under vacuum. The residue was purified by a prep-TLC with DCM:MeOH=52:1 to afford 140 mg of the title compound as a yellow solid. LC-MS: (ES, m/z): RT=0.869 min, m/z=475 [M+1].

### Intermediate BP-10: (S)—N-(7-bromo-5-(piperidin-3-yloxy)quinazolin-4-yl)benzo[d]thiazol-6-amine

### Intermediate BP-10a: tert-butyl (S)-3-((4-(benzo[d]thiazol-6-ylamino)-7-bromoquinazolin-5-yl)oxy)piperidine-1-carboxylate

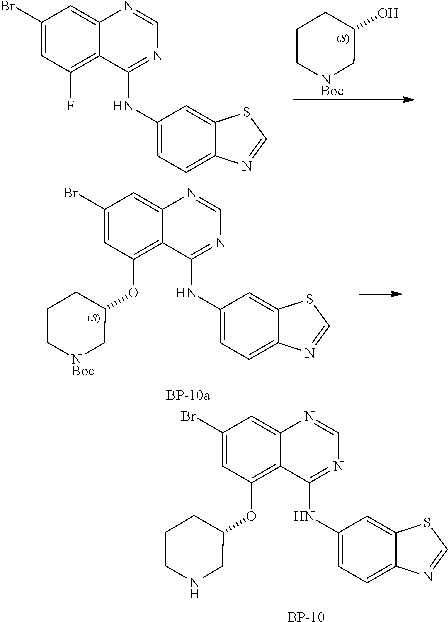

### Step I: Synthesis of Intermediate BP-10a: tert-butyl (S)-3-((4-(benzo[d]thiazol-6-ylamino)-7-bromoquinazolin-5-yl)oxy)piperidine-1-carboxylate

NaH (169 mg, 4.24 mmol) was added to tert-butyl (3R)-3-hydroxypiperidine-1-carboxylate (640 mg, 3.18 mmol) in DMF (15 ml) at 0° C. After stirring for 30 min, Intermediate BP-59 (400 mg, 1.06 mmol) was added and the mixture was stirred for 100° C. for 16 h. The reaction mixture was diluted with EA (100 mL), and washed with ice water (100 mL×3) and saturated brine (100 mL). The organic layer was dried over Na₂SO₄, filtered and concentrated. The crude product was purified by prep-TLC with DCM:MeOH=20:1. The resulted in 240 mg (40.7%) as a light yellow solid. LC-MS: (ES, m/z): RT=0.756 min, LCMS: m/z=556 [M+1],

### Step 2: (S)—N-(7-bromo-5-(piperidin-3-yloxy)quinazolin-4-yl)benzo[d]thiazol-6-amine

TFA (98.2 mg, 862 μmol) was added to Intermediate BP-10a (240 mg, 0.4312 mmol) in DCM (8 ml) at rt. The resulting solution was stirred at room temperature for 3 h. The reaction mixture was diluted with DCM (100 mL), and washed sequentially with water (100 mL*3) and saturated brine (100 mL*1). The organic layer was dried over Na₂SO₄, filtered and concentrated. The crude product was purified by prep-TLC with DCM:MeOH=20:1 to afford the title compound (180 mg, 91.8%) as a light yellow solid. LC-MS: (ES, m/z): RT=0.805 min, LCMS: m/z=456 [M+1].

### Intermediate BP-11: N-(1,3-benzothiazol-6-yl)-7-bromo-5-(oxan-4-yloxy)quinazolin-4-amine

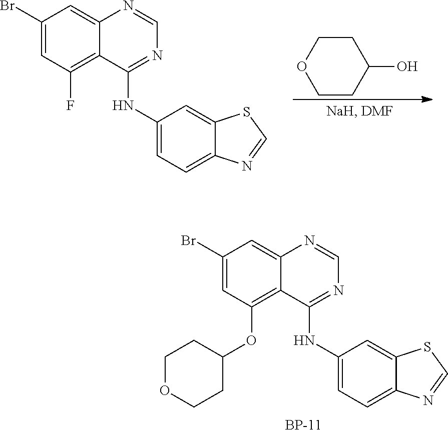 [SMILES: Fc1cc(Br)cc2ncnc(Nc3ccc4ncsc4c3)c12 ; OC1CCOCC1 ; Brc1cc(OC2CCOCC2)c2c(Nc3ccc4ncsc4c3)ncnc2c1 ; "BP-11"]

To a solution of oxan-4-ol (815 mg, 2.66 mmol) in DMF (10 ml), NaH (211 mg, 5.32 mmol) was added at 0° C. for 0.5 h, N-(1,3-benzothiazol-6-yl)-7-bromo-5-fluoroquinazolin-4-amine (1 g, 2.66 mmol) was added and warmed to 100° C. for 4 h. The reaction mixture was diluted with water and extracted with EA and saturated brine. The organic layer was dried over Na₂SO₄, filtered, evaporated, and purified by column chromatography (DCM:MeOH=15:1) to afford N-(1,3-benzothiazol-6-yl)-7-bromo-5-(oxan-4-yloxy)quinazolin-4-amine (800 mg, 66.1%) as a yellow solid. LC-MS: (ES, m/z): RT=2.070 min, LCMS: m/z=457 [M+1].

### Intermediate BP-12: (S)-1-(dimethylamino)-3-methoxypropan-2-ol

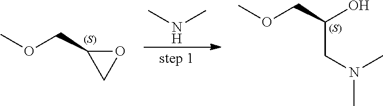

To 15 mL of Me₂NH₂ (aq) was added (2S)-2-(methoxymethyl)oxirane (1 g, 11.3 mmol). Stirred at rt. for 2 hours. The resulting solution was extracted with DCM. The organic layer was separated and dried over Na₂SO₄, filtered and concentrated to afford (S)-1-(dimethylamino)-3-methoxypropan-2-ol (1.2 g, yield: 79%) as a light-yellow solid. LC-MS: (ES, m/z): RT=0.182 min, LCMS: m/z=134 [M+1], ¹H NMR (300 MHz, Chloroform-d) δ 3.87 (m, 1H), 3.49-3.33 (m, 5H), 2.47 (dd, J=12.3, 10.0 Hz, 1H), 2.31 (s, 6H), 2.25 (dd, J=12.2, 3.6 Hz, 1H),

### Intermediate BP-13: rac-3-(dimethylamino)butan-2-ol and (2S,3S)-3-(dimethylamino)butan-2-ol

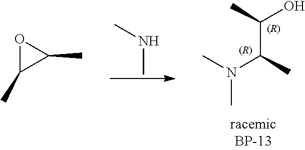

Dimethylamine (6 mL, 33% in water) was added to (2R,3S)-2,3-dimethyloxirane (1.0 g, 13.8 mmol) at 0° C. The resulting mixture was stirred at 45° C. for 16 h. The mixture was diluted with DCM 100 mL and washed with brine 10 mL*2. The organic layer was dried with Na₂SO₄ and concentrated under vacuum. The residue was purified by a silica gel chromatography with PE:EA=2:1 to afford 1.0 g of the title compound as a colorless oil. ¹H NMR (400 MHz, DMSO-d6) δ 4.23 (s, 1H), 3.38 (dq, J=8.2, 6.1 Hz, 1H), 2.18 (dq, J=8.3, 6.6 Hz, 1H), 2.13 (s, 6H), 0.98 (d, J=6.1 Hz, 3H), 0.78 (d, J=6.6 Hz, 3H),

### Intermediate BP-14: rac-3-(dimethylamino)butan-2-ol and (2S,3R)-3-(dimethylamino)butan-2-ol

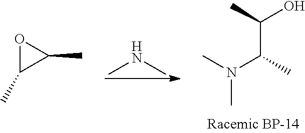

Dimethylamine (6 mL, 33% in water) was added to (1R,2R)-1,2-dimethylcyclopropane (1.0 g, 13.8 mmol) at 0 degree. The resulting mixture was stirred at 45 degrees for 16 h. The mixture was diluted with DCM 100 mL and washed with brine 10 mL*2, the organic layer was dried with Na₂SO₄ and concentrated under vacuum. The residue was purified by a silica gel chromatography with PE:EA=2:1 to afford the title compound (1.0 g) as a colorless oil. ¹H NMR (300 MHz, DMSO-d6) δ 4.28 (s, 1H), 3.52 (p, J=6.4 Hz, 1H), 2.19 (q, J=6.7 Hz, 1H), 2.12 (s, 6H), 1.07 (d, J=6.2 Hz, 3H), 0.88 (d, J=6.6 Hz, 3H),

### Intermediate BP-15: N-(7-bromo-5-((1-(dimethylamino)propan-2-yl)oxy)quinazolin-4-yl)benzo[d]thiazol-6-amine

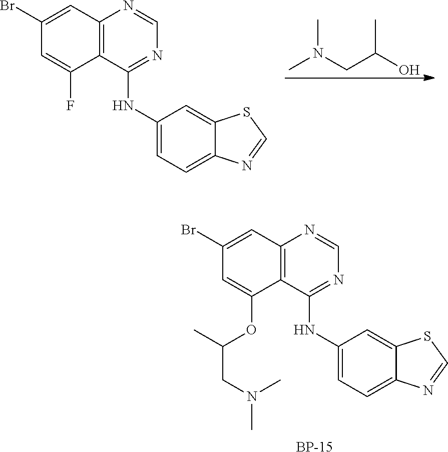

To a solution of 1-dimethylamino-2-propanol (242 mg, 2.34 mmol) in 5 ml of THF was added potassium t-butoxide (4.51 ml, 4.51 mmol). The reaction was stirred for 10 min then N-(7-bromo-5-fluoroquinazolin-4-yl)benzo[d]thiazol-6-amine (676.6 mg, 1.803 mmol) was added. Stirred at 90° C. for 10 hr. The reaction was concentrated to give a crude solid. Triturated the crude product with 5 ml of EtOAc and 3 ml of Heptane. The solid was collected to afford the title compound. LC-MS: (ES, m/z): RT=2.125 min, LC-MS: m/z=458.

### Intermediate BP-16: tert-butyl (S)-3-(4-(4,4,5,5-tetramethyl-1,3,2-dioxaborolan-2-yl)-1H-pyrazol-1-yl)pyrrolidine-1-carboxylate

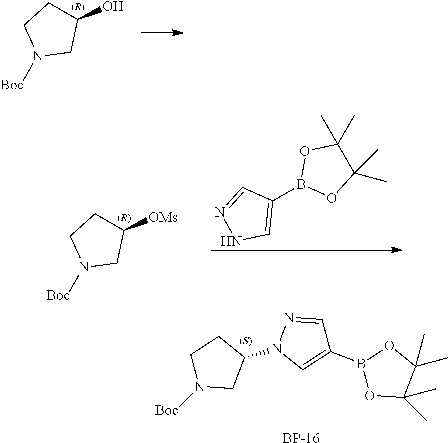

### Step 1: (R)-tert-butyl 3-((methylsulfonyl)oxy)pyrrolidine-1-carboxylate

To a mixture of (R)-tert-butyl 3-hydroxypyrrolidine-1-carboxylate (10 g, 53.41 mmol, 1 eq) in DCM (100 mL) was added TEA (16.21 g, 160.23 mmol, 22.30 mL, 3 eq). Methanesulfonyl chloride (9.18 g, 80.11 mmol, 6.20 mL, 1.5 eq) was added dropwise at 0° C. The reaction mixture was stirred at 25° C. for 12 hrs. The reaction mixture was added to the ice water (100 ml), then The reaction mixture was diluted with EA (100 mL×3), saturated brine (100 mL), the combined organic phase was dried over Na2SO4, filtered and concentrated to afford the title compound (15 g, crude) as a brown oil. 1H NMR (400 MHz, DMSO-d6) δ 5.25 (s, 1H), 3.49 (s, 2H), 3.45-3.38 (m, 1H), 3.33-3.26 (m, 1H), 3.23 (s, 3H), 2.12 (s, 2H), 1.41 (s, 9H).

### Step 2: tert-butyl (S)-3-(4-(4,4,5,5-tetramethyl-1,3,2-dioxaborolan-2-yl)-1H-pyrazol-1-yl)pyrrolidine-1-carboxylate

To a mixture of 4-(4,4,5,5-tetramethyl-1,3,2-dioxaborolan-2-yl)-1H-pyrazole (1 g, 5.15 mmol, 1 eq) in DMF (10 mL) was added NaH (309.19 mg, 7.73 mmol, 60% purity, 1.5 eq) at 0° C. The reaction mixture was stirred at 0° C. for 15 min. (R)-tert-butyl 3-((methylsulfonyl)oxy)pyrrolidine-1-carboxylate (2.05 g, 7.73 mmol, 1.5 eq) was added and the reaction was stirred at 100° C. for 3 hrs. The reaction mixture was added to the ice water (20 ml). The reaction mixture was diluted with EA (20 mL), washed with water (20 mL×3) and saturated brine (50 mL). The combined organic phase was dried over Na₂SO₄, filtered and evaporated to afford the title compound (1.5 g, crude) as a yellow oil. LC-MS: (ES, m/z): RT=2.544 min, LC-MS: m/z=364.2 [M+1].

### Intermediate BP-17: tert-butyl 4-(3-methoxy-4-(4,4,5,5-tetramethyl-1,3,2-dioxaborolan-2-yl)-1H-pyrazol-1-yl)piperidine-1-carboxylate

### Intermediate BP-17a: tert-butyl 4-(4-bromo-3-methoxy-1H-pyrazol-1-yl)piperidine-1-carboxylate

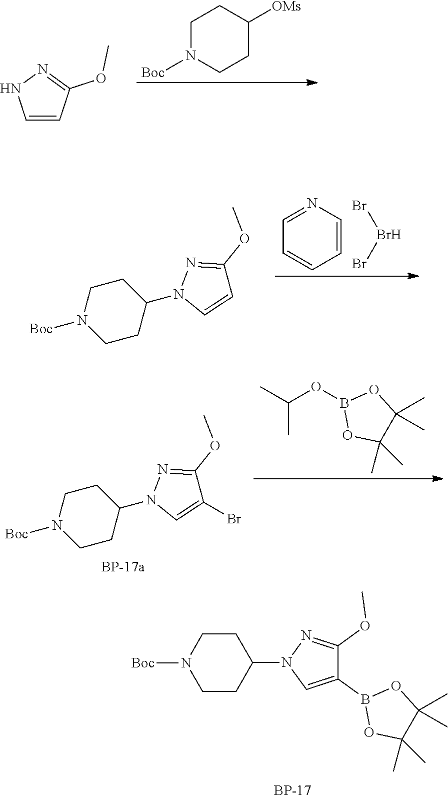

### Step 1: tert-butyl 4-(3-methoxy-1H-pyrazol-1-yl)piperidine-1-carboxylate

To a mixture of 3-methoxy-1H-pyrazole (6.5 g, 66.26 mmol, 1 eq) and tert-butyl 4-((methylsulfonyl)oxy)piperidine-1-carboxylate (27.76 g, 99.39 mmol, 1.5 eq) in DMF (100 mL) was added Cs₂CO₃ (32.38 g, 99.39 mmol, 1.5 eq) and stirred at 100° C. for 12 hrs. The mixture was concentrated and the residue purified by prep-HPLC (column: Luna C18 100*30 5 u; mobile phase: [water (0.2% FA)-ACN]; B %: 45%-55%, 12 min) to afford the title compound (9 g, 31.99 mmol) as a yellow oil. LC-MS: (ES, m/z): RT=2.399 min, LC-MS: m/z=282.2 [M+1].

### Step 2: tert-butyl 4-(4-bromo-3-methoxy-1H-pyrazol-1-yl)piperidine-1-carboxylate

To a mixture of tert-butyl 4-(3-methoxy-1H-pyrazol-1-yl)piperidine-1-carboxylate (9 g, 31.99 mmol, 1 eq) in MeOH (100 mL) was added pryridinium tribromide (10.23 g, 31.99 mmol, 1 eq) at 0° C. under N₂. The mixture was stirred at rt for 12 hrs. The mixture was concentrated and the residue was purified by silica gel chromatography eluting with a gradient of PE to 50% EA in PE. Fractions were concentrated to afford the title compound (10 g, 27.76 mmol) as a white solid. LC-MS: (ES, m/z): RT=1.543 min, LC-MS: m/z=360.1 [M+1].

### Step 3: tert-butyl 4-(3-methoxy-4-(4,4,5,5-tetramethyl-1,3,2-dioxaborolan-2-yl)-1H-pyrazol-1-yl)piperidine-1-carboxylate

To a mixture of tert-butyl 4-(4-bromo-3-methoxy-1H-pyrazol-1-yl)piperidine-1-carboxylate (3 g, 8.33 mmol, 1 eq) in THF (30 mL) was added n-BuLi (2.5 M, 4.00 mL, 1.2 eq) dropwise at −70° C. After addition, the mixture was stirred at −70° C. for 0.2 hrs then 2-isopropoxy-4,4,5,5-tetramethyl-1,3,2-dioxaborolane (3.10 g, 16.66 mmol, 3.40 mL, 2 eq) was added. The reaction was stirred at rt for 1 hr. The mixture was quenched with MeOH (10 mL), stirred at rt for 10 mins, filtered and evaporated to afford the title compound (5 g, crude) as a yellow solid. LC-MS: (ES, m/z): RT=1.252 min, LC-MS: m/z=408.3 [M+1].

### Intermediate BP-18: tert-butyl (R)-3-(4-bromo-3-methoxy-1H-pyrazol-1-yl)pyrrolidine-1-carboxylate

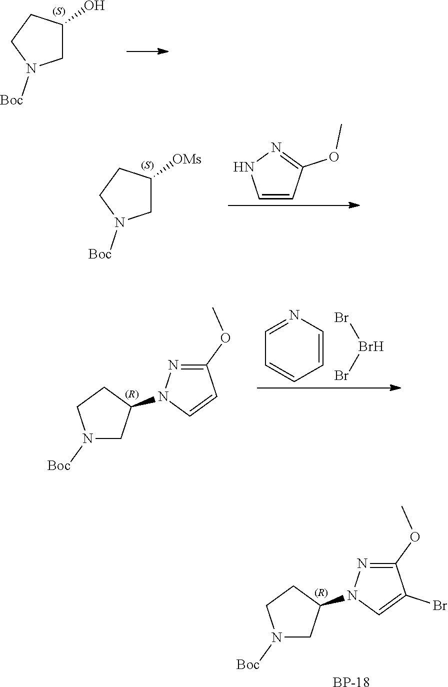

### Step 1: (S)-tert-butyl 3-((methylsulfonyl)oxy)pyrrolidine-1-carboxylate

To the mixture of (S)-tert-butyl 3-hydroxypyrrolidine-1-carboxylate (10 g, 53.41 mmol, 1 eq) in DCM (100 mL) was added TEA (16.21 g, 160.23 mmol, 22.30 mL, 3 eq). MsCl (9.18 g, 80.11 mmol, 6.20 mL, 1.5 eq) was added dropwise at 0° C. The reaction was stirred at rt for 12 hrs then washed with water and saturated brine. The organic layer was dried over Na₂SO₄, filtered and evaporated to afford the title compound (13 g, crude) as a yellow oil. ¹H NMR (400 MHz, DMSO-d6) δ 5.25 (s, 1H), 3.53-3.49 (m, 2H), 3.42 (m, 1H), 3.29 (d, 1H, J=8.4 Hz), 3.24 (s, 3H), 2.13 (s, 2H), 1.41 (s, 9H),

### Step 2: (R)-tert-butyl 3-(3-methoxy-1H-pyrazol-1-yl)pyrrolidine-1-carboxylate

To the mixture of 3-methoxy-1H-pyrazole (500 mg, 5.10 mmol, 1 eq) and (S)-tert-butyl 3-((methylsulfonyl)oxy)pyrrolidine-1-carboxylate (1.62 g, 6.12 mmol, 1.2 eq) in DMF (4 mL) was added Cs₂CO₃ (2.49 g, 7.65 mmol, 1.5 eq) at rt. The reaction was heated at 100° C. for 12 hrs then concentrated under reduced pressure. The crude product was purified by silica gel chromatography eluting with a gradient of PE to 20% EA in PE. Fractions were evaporated to afford the title compound (900 mg, crude) as a yellow oil. LC-MS: (ES, m/z): RT=1.156 min, LC-MS: m/z=268.1 [M+1]. ¹HNMR (400 MHz, Chloroform-d) 8.88 (d, 1H, J=1.2 Hz), 8.71 (s, 1H), 8.60 (t, 1H, J=8.4 Hz), 8.12 (s, 1H), 7.96 (d, 1H, J=9.2 Hz), 7.87-7.80 (m, 2H), 7.64 (s, 1H), 3.99 (s, 3H), 3.96-3.92 (m, 1H), 2.98 (d, 2H, J=11.2 Hz), 2.31 (s, 3H), 2.14 (d, 4H, J=7.6 Hz), 2.08-1.97 (m, 2H),

### Step 3: tert-butyl (R)-3-(4-bromo-3-methoxy-1H-pyrazol-1-yl)pyrrolidine-1-carboxylate

To the mixture of (R)-tert-butyl 3-(3-methoxy-1H-pyrazol-1-yl)pyrrolidine-1-carboxylate (900 mg, 3.37 mmol, 1 eq) in MeOH (15 mL) was added pyridinium tribromide (1.08 g, 3.37 mmol, 1 eq) at 0° C. under N₂. The mixture was stirred at 20° C. for 2 hrs. Then the reaction mixture was concentrated under reduced pressure. The residue was dissolved in DCM (15 mL) and TEA (1.02 g, 10.10 mmol, 1.41 mL, 3 eq) and Boc₂O (1.10 g, 5.05 mmol, 1.16 mL, 1.5 eq) were added. The resulting mixture was stirred at rt for 1 hr. The mixture was concentrated under reduced pressure. The crude product was purified by silica gel chromatography eluting with a gradient of PE to 20% EA in PE. Fractions were concentrated to afford the title compound (700 mg, 2.02 mmol, 60.05% yield) as a yellow solid. LC-MS: (ES, m/z): RT=1.464 min, LC-MS: m/z=348.0 [M+1], ¹H NMR (400 MHz, Chloroform-d) δ 7.18 (s, 1H), 4.58-4.54 (m, 1H), 3.86 (s, 3H), 3.67 (s, 1H), 3.55-3.50 (m, 1H), 3.42 (s, 2H), 2.25-2.15 (m, 2H), 1.40 (s, 9H),

### Intermediate BP-19: tert-butyl 4-((4-chloro-7-methoxyquinazolin-6-yl)oxy)piperidine-1-carboxylate

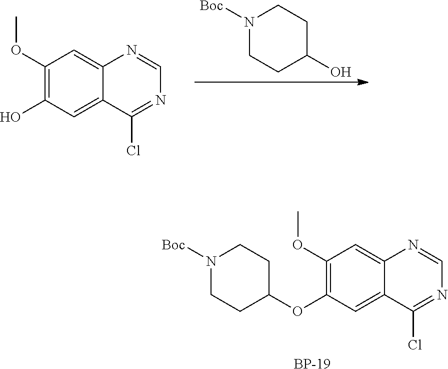 [SMILES: COc1cc2ncnc(Cl)c2cc1O ; CC(C)(C)OC(=O)N1CCC(O)CC1 ; COc1cc2ncnc(Cl)c2cc1OC1CCN(C(=O)OC(C)(C)C)CC1 ; "BP-19"]

To a solution of 4-chloro-7-methoxy-quinazolin-6-ol (550 mg, 2.61 mmol, 1 eq) in DCM (3 mL) was added tert-butyl 4-hydroxypiperidine-1-carboxylate (788.36 mg, 3.92 mmol, 1.5 eq) and PPh₃ (1.03 g, 3.92 mmol, 1.5 eq) under N₂. A solution of DBAD (901.95 mg, 3.92 mmol, 1.5 eq) in DCM (3 mL) was added to the reaction mixture at 0° C. The mixture was stirred at 20° C. for 12 hrs under N₂. The reaction mixture was filtered and concentrated under reduced pressure to afford crude product. The crude product was purified by silica gel chromatography eluting with a gradient of PE to 30% EA in PE to afford the title compound (1.1 g, crude) as a pale yellow solid. LC-MS: (ES, m/z): RT=1.513 min, LC-MS: m/z=393.1 [M+1].

### Intermediate BP-20: 4-bromo-3-methoxy-1-(2-(methylsulfonyl)ethyl)-1H-pyrazole

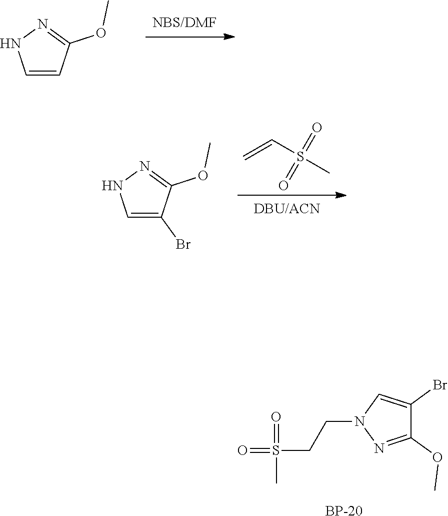

### Step 1: 4-bromo-3-methoxy-1H-pyrazole

To a solution of 3-methoxy-1H-pyrazole (500 mg, 5.10 mmol, 1 eq) in DMF (2 mL) was added NBS (907.13 mg, 5.10 mmol, 1 eq) at 25° C. and stirred for 2 hrs. The mixture was concentrated and the residue was purified by prep-HPLC (column: Kromasil C18 (250*50 mm*10 um); mobile phase: [water (0.04% NH₃H₂O+10 mM NH₄HCO₃)-ACN]; B %: 15%-35%, 10 min) to afford the title compound (400 mg, 2.26 mmol) as a white solid. LC-MS: (ES, m/z): RT=1.183 min, LC-MS: m/z=177.0 [M+1].

### Step 2: 4-bromo-3-methoxy-1-(2-(methylsulfonyl)ethyl)-1H-pyrazole

To a solution of (methylsulfonyl)ethene (359.81 mg, 3.39 mmol, 297.36 uL, 1.5 eq) and 4-bromo-3-methoxy-1H-pyrazole (400 mg, 2.26 mmol, 1 eq) in ACN (2 mL) was added DBU (172.02 mg, 1.13 mmol, 170.32 uL, 0.5 eq) at 25° C., the reaction stirred at 90° C. for 12 hrs. The mixture was concentrated and the crude product was purified by silica gel chromatography eluting with a gradient of PE to 20% EA in PE to afford the title compound (400 mg, 1.41 mmol) as a white solid. LC-MS: (ES, m/z): RT=1.453 min, LC-MS: m/z=283.0 [M+1]. ¹H NMR (400 MHz, DMSO-d6) δ 7.86 (s, 1H), 4.37 (d, 2H, J=6.8 Hz), 3.826 (s, 3H), 3.600 (d, 2H, J=6.8 Hz), 2.93 (s, 3H)

### Intermediate BP-23: 7-fluorobenzo[d]thiazol-6-amine

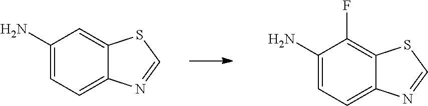 [SMILES: Nc1ccc2ncsc2c1 ; Nc1ccc2ncsc2c1F]

To a solution of 1,3-benzothiazol-6-amine (2 g, 13.32 mmol, 1 eq) in ACN (4 mL) was added selectfluor (4.72 g, 13.32 mmol, 1 eq) at 0° C., the reaction was stirred at 25° C. for 1 hr. The reaction was concentrated. The mixture was purified by prep-HPLC (column: Welch Xtimate C18 250*50 mm*10 um; mobile phase: [water (0.04% NH₃H₂O+10 mM NH₄HCO₃)-ACN]; B %: 15%-40%, 10 min) to afford the title compound (500 mg, 2.97 mmol, 22.33% yield) as a brown solid. LC-MS: (ES, m/z): RT=0.896 min, LC-MS: m/z=168.0 [M+1], ¹H NMR (400 MHz, DMSO-d) δ 9.01 (s, 1H), 7.64 (d, 1H, J=8 Hz), 7.02 (t, 1H, J=8.8 Hz), 5.46 (s, 2H),

### Intermediate BP-24: tert-butyl (S)-3-(3-methoxy-4-(4,4,5,5-tetramethyl-1,3,2-dioxaborolan-2-yl)-1H-pyrazol-1-yl)pyrrolidine-1-carboxylate

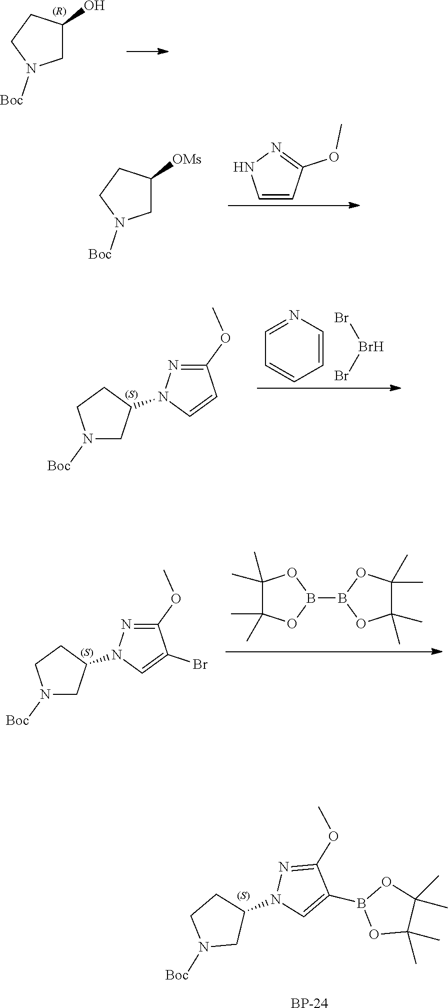

### Step 1: (R)-tert-butyl 3-((methylsulfonyl)oxy)pyrrolidine-1-carboxylate

To a mixture of (R)-tert-butyl 3-hydroxypyrrolidine-1-carboxylate (10 g, 53.41 mmol, 1 eq) in DCM (100 mL) was added TEA (16.21 g, 160.23 mmol, 22.30 mL, 3 eq). Methanesulfonyl chloride (9.18 g, 80.11 mmol, 6.20 mL, 1.5 eq) was added dropwise at 0° C., the reaction mixture was stirred at 25° C. for 12 hrs. The reaction mixture was added to ice water (100 ml). The mixture was diluted with EA (100 mL×3) and washed with saturated brine (100 mL). The combined organic phase was dried over Na₂SO₄, filtered and concentrated to afford the title compound (15 g, crude) as a brown oil. ¹H NMR (400 MHz, DMSO-d₆) δ 5.25 (s, 1H), 3.49 (s, 2H), 3.45-3.38 (m, 1H), 3.33-3.26 (m, 1H), 3.23 (s, 3H), 2.12 (s, 2H), 1.41 (s, 9H),

### Step 2: (S)-tert-butyl 3-(3-methoxy-1H-pyrazol-1-yl)pyrrolidine-1-carboxylate

To a mixture of 3-methoxy-1H-pyrazole (500 mg, 5.10 mmol, 1 eq) and (R)-tert-butyl 3-((methylsulfonyl)oxy)pyrrolidine-1-carboxylate (2.71 g, 10.20 mmol, 2 eq) in DMF (5 mL) was added Cs₂CO₃ (2.49 g, 7.65 mmol, 1.5 eq) at 25° C. and the reaction mixture was stirred at 100° C. for 16 hrs. The reaction mixture was filtered and the filtrate concentrated. The crude product was purified by silica gel chromatography eluting with a gradient of PE to 20% EA in PE. Fractions were concentrated to afford the title compound (0.4 g, 1.50 mmol, 29.34% yield) as a yellow oil. LC-MS: (ES, m/z): RT=2.090 min, LC-MS: m/z=268.2 [M+1]. ¹H NMR (400 MHz, Chloroform-d) δ 7.13 (d, 1H, J=2.4 Hz), 5.57 (d, 1H, J=2.4 Hz), 4.60 (m, 1H), 3.79 (s, 3H), 3.68 (s, 1H), 3.62-3.35 (m, 3H), 2.37-2.08 (m, 2H), 1.40 (s, 9H),

### Step 3: (S)-tert-butyl 3-(4-bromo-3-methoxy-1H-pyrazol-1-yl)pyrrolidine-1-carboxylate

To a mixture of (S)-tert-butyl 3-(3-methoxy-1H-pyrazol-1-yl)pyrrolidine-1-carboxylate (400.00 mg, 1.50 mmol, 1 eq) in MeOH (4 mL) was added pyridinium tribromide (478.55 mg, 1.50 mmol, 1 eq) at 0° C. under N₂. The reaction was stirred at 25° C. for 1 hr. The reaction mixture was concentrated under reduced pressure. The residue was dissolved in DCM (4 mL). TEA (454.23 mg, 4.49 mmol, 624.80 uL, 3 eq) and (Boc)₂O (489.85 mg, 2.24 mmol, 515.63 uL, 1.5 eq) were added and the reaction mixture was stirred at 25° C. for 1 hr. The reaction mixture was concentrated under reduced pressure. The crude product was purified by silica gel chromatography eluting with a gradient of PE to 10% EA in PE. Fractions were concentrated to afford the title compound (0.3 g, 866.50 umol, 57.91% yield) as a yellow solid. LC-MS: (ES, m/z): RT=1.307 min, LC-MS: m/z=346.2 [M+1]. ¹H NMR (400 MHz, Chloroform-d) 7.18 (s, 1H), 4.64-4.50 (m, 1H), 3.86 (s, 3H), 3.68-3.67 (m, 1H), 3.59-3.36 (m, 3H), 2.30-2.13 (m, 2H), 1.40 (s, 9H)

### Step 4: tert-butyl (S)-3-(3-methoxy-4-(4,4,5,5-tetramethyl-1,3,2-dioxaborolan-2-yl)-1H-pyrazol-1-yl)pyrrolidine-1-carboxylate

To a mixture of (S)-tert-butyl 3-(4-bromo-3-methoxy-1H-pyrazol-1-yl)pyrrolidine-1-carboxylate (0.3 g, 866.50 umol, 1 eq) and 4,4,5,5-tetramethyl-2-(4,4,5,5-tetramethyl-1,3,2-dioxaborolan-2-yl)-1,3,2-dioxaborolane (1.10 g, 4.33 mmol, 5 eq) in dioxane (2 mL) was added XPhos Pd G₂ (68.18 mg, 86.65 umol, 0.1 eq) and KOAc (170.08 mg, 1.73 mmol, 2 eq) at 25° C. The reaction mixture was stirred at 90° C. for 5 hrs under N₂. The reaction mixture was filtered and the filtrate was concentrated. The crude product was purified by silica gel chromatography eluting with a gradient of PE to 5% EA in PE. Fractions were concentrated to afford the title compound (0.3 g, 762.81 umol, 88.03% yield) as a white solid. LC-MS: (ES, m/z): RT=2.657 min, LC-MS: m/z=394.2 [M+1]. ¹H NMR (400 MHz, Chloroform-d) δ 7.44 (s, 1H), 4.59 (d, 1H, J=6.0 Hz), 3.85 (s, 3H), 3.68 (s, 1H), 3.60-3.36 (m, 3H), 2.35-2.16 (m, 2H), 1.24 (s, 9H), 1.20-1.19 (m, 6H), 1.18-1.16 (m, 6H)

### Intermediate BP-25: tert-butyl (R)-3-(4-(4,4,5,5-tetramethyl-1,3,2-dioxaborolan-2-yl)-1H-pyrazol-1-yl)pyrrolidine-1-carboxylate

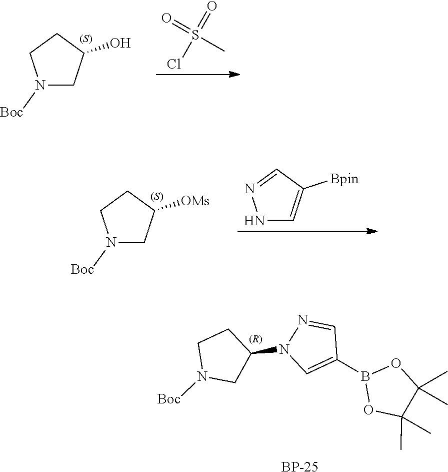

### Step 1: (S)-tert-butyl 3-((methylsulfonyl)oxy)pyrrolidine-1-carboxylate

To the mixture of tert-butyl (3S)-3-hydroxypyrrolidine-1-carboxylate (10 g, 53.41 mmol, 1 eq) in DCM (100 mL) was added TEA (16.21 g, 160.23 mmol, 22.30 mL, 3 eq). Then MsCl (9.18 g, 80.11 mmol, 6.20 mL, 1.5 eq) was added dropwise at 0° C. The mixture was stirred at 20° C. for 12 hrs. The mixture was washed with H₂O (80 mL*3) and the combined organic layer were dried over Na₂SO₄, filtered and concentrated to afford the title compound (13 g, crude) as yellow oil. ¹H NMR (400 MHz, DMSO-d6) δ 5.25 (s, 1H), 3.53-3.49 (m, 2H), 3.42-3.40 (m, 1H), 3.29 (d, 1H, J=8.4 Hz), 3.24 (s, 3H), 2.13 (s, 2H), 1.41 (s, 9H),

### Step 2: tert-butyl (R)-3-(4-(4,4,5,5-tetramethyl-1,3,2-dioxaborolan-2-yl)-1H-pyrazol-1-yl)pyrrolidine-1-carboxylate

To a mixture of 4-(4,4,5,5-tetramethyl-1,3,2-dioxaborolan-2-yl)-1H-pyrazole (1.5 g, 7.73 mmol, 1 eq) in DMF (15 mL) was added NaH (309.19 mg, 7.73 mmol, 60% purity, 1 eq) at 0° C. The reaction was stirred at 0° C. for 0.5 hr. (S)-tert-butyl 3-((methylsulfonyl)oxy)pyrrolidine-1-carboxylate (2.05 g, 7.73 mmol, 1 eq) was added to the reaction mixture at 0° C. The reaction was heated to 100° C. for 2 hrs. H₂O (50 ml) was added and the mixture extracted with EA (50 mL*3). The combined organic layers were dried over Na₂SO₄, filtered and concentrated to afford the title compound (2 g, crude) as a yellow solid. LC-MS: (ES, m/z): RT=2.551 min, LC-MS: m/z=363.2 [M+1].

### Intermediate BP-26: N4-(benzo[d]thiazol-6-yl)-7-(1-methyl-1H-pyrazol-3-yl)quinazoline-4,6-diamine

### Intermediate BP-26b: N-(7-bromo-6-nitroquinazolin-4-yl)benzo[d]thiazol-6-amine

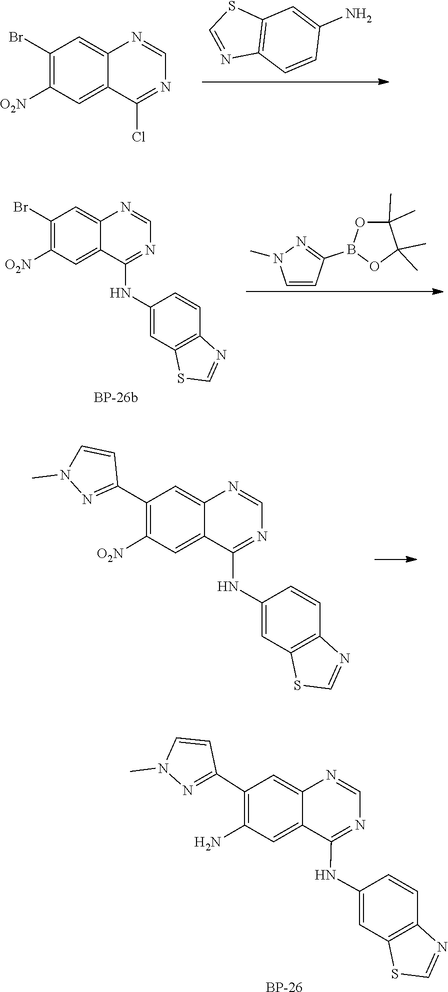

### Step 1: N-(7-bromo-6-nitroquinazolin-4-yl)benzo[d]thiazol-6-amine

To the mixture of 7-bromo-4-chloro-6-nitro-quinazoline (2 g, 6.93 mmol, 1 eq) in IPA (20 mL) was added 1,3-benzothiazol-6-amine (1.15 g, 7.63 mmol, 1.1 eq) at rt. The mixture was heated at 90° C. for 2 hrs. The reaction mixture was filtered and the solid collected to afford the title compound (2.9 g, crude) as a yellow solid.

### Step 2: N-(7-(1-methyl-1H-pyrazol-3-yl)-6-nitroquinazolin-4-yl)benzo[d]thiazol-6-amine

N-(7-bromo-6-nitro-quinazolin-4-yl)-1,3-benzothiazol-6-amine (3.5 g, 8.70 mmol, 1 eq), 1-methyl-3-(4,4,5,5-tetramethyl-1,3,2-dioxaborolan-2-yl)pyrazole (3.62 g, 17.40 mmol, 2 eq), DMSO (20 mL), H₂O (5 mL), XPhos Pd G₂ (684.64 mg, 870.16 umol, 0.1 eq) and K₃PO₄ (3.69 g, 17.40 mmol, 2 eq) were combined at rt. The reaction was heated at 80° C. for 4 hrs under N₂. The reaction mixture was diluted with H₂O (30 mL), filtered, the solid collected and washed with EA (50 mL) to afford the title compound (4 g, crude) as a brown solid. LC-MS: (ES, m/z): RT=1.096 min, LC-MS: m/z=404.0 [M+1].

### Step 3: N4-(benzo[d]thiazol-6-yl)-7-(1-methyl-1H-pyrazol-3-yl)quinazoline-4,6-diamine

N-[7-(1-methylpyrazol-3-yl)-6-nitro-quinazolin-4-yl]-1,3-benzothiazol-6-amine (4 g, 9.92 mmol, 1 eq), Fe (2.77 g, 49.58 mmol, 5 eq) and NH₄Cl (2.65 g, 49.58 mmol, 5 eq) were combined in THF (50 mL) and H₂O (15 mL) at rt. The mixture was heated at 70° C. for 5 hrs. The reaction mixture was filtered and the filtrate was concentrated. The residue was diluted with EA (100 mL), washed with water (50 mL) and saturated brine (50 mL). The organic layer was dried over Na₂SO₄, filtered and evaporated to afford the title compound (2.2 g, 5.89 mmol, 59.42% yield) as a yellow solid. LC-MS: (ES, m/z): RT=1.021 min, LC-MS: m/z=374.0 [M+1], ¹H NMR (400 MHz, DMSO-d) δ 11.27 (s, 1H), 9.45 (s, 1H), 8.74 (s, 1H), 9.59 (s, 1H), 8.58-8.21 (m, 1H), 8.09 (s, 1H), 7.87 (s, 1H), 7.85-7.82 (s, 2H), 6.90 (s, 1H), 4.06 (s, 3H),

### Intermediate BP-26a: N4-(7-fluorobenzo[d]thiazol-6-yl)-7-(1-methyl-1H-pyrazol-3-yl)quinazoline-4,6-diamine

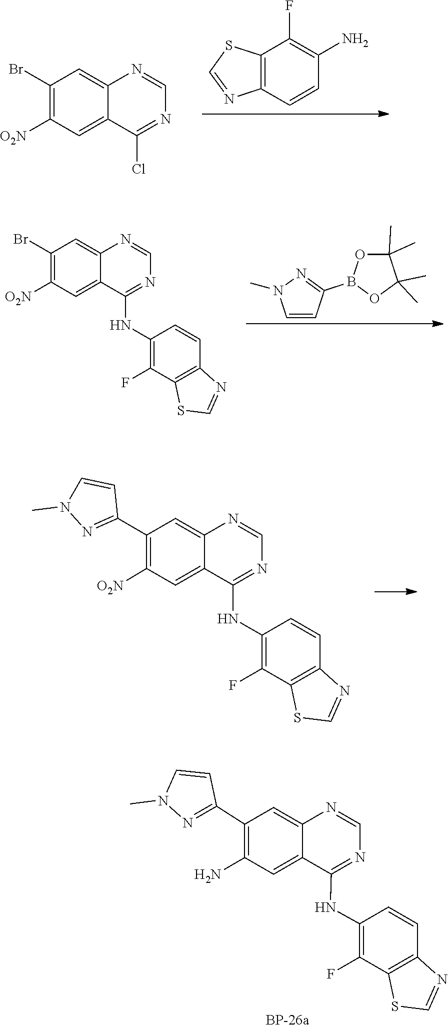

### Intermediate BP-26a was Prepared Using the Method of BP-26, Replacing benzo[d]thiazol-6-amine with 7-fluorobenzo[d]thiazol-6-amine in Step 1

### Intermediate BP-27: (3-methoxy-1-methyl-1H-pyrazol-5-yl)boronic acid

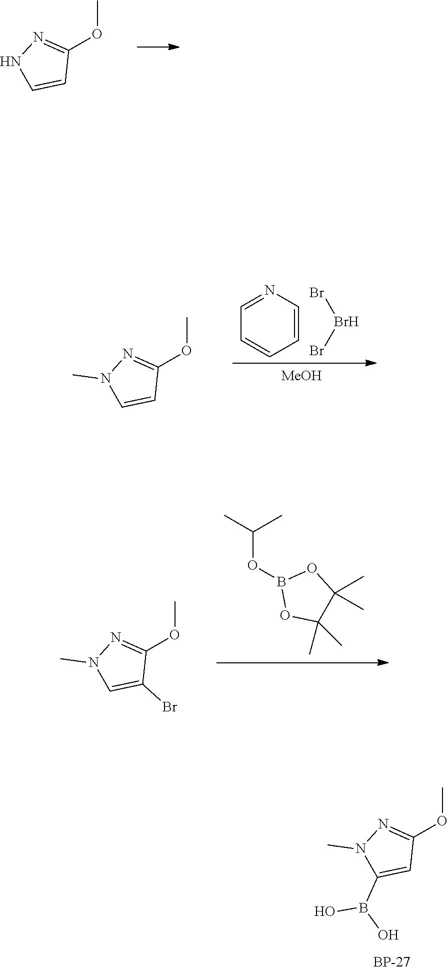

### Step 1: 3-methoxy-1-methyl-1H-pyrazole

To a mixture of 3-methoxy-1H-pyrazole (2.00 g, 20.39 mmol, 1.00 eq), Cs₂CO₃ (13.28 g, 40.77 mmol, 2.00 eq) and DMF (20.00 mL) was added CH₃I (3.47 g, 24.46 mmol, 1.52 mL, 1.20 eq). The reaction mixture was stirred at 25° C. for 3 hrs. The reaction mixture was diluted with EA (100 mL) and washed with water (100 mL×3). The combined organic layers were dried over Na₂SO₄, filtered and evaporated to afford crude product which was purified by silica gel chromatography, eluting with a gradient of PE to 5% EA in PE. Fractions were concentrated to afford the title compound (1.45 g, crude) as colorless liquid. LC-MS: (ES, m/z): RT=0.670 min, LC-MS: m/z=113.1 [M+1].

### Step 2: 4-bromo-3-methoxy-1-methyl-1H-pyrazole

To a solution of 3-methoxy-1-methyl-1H-pyrazole (1.45 g, 12.93 mmol, 1.00 eq) in MeOH (20.00 mL) was added Pyridinium tribromide (4.14 g, 12.93 mmol, 1.00 eq) at 0° C. under N₂. The mixture was stirred at 25° C. for 12 hrs. The reaction mixture was concentrated under reduced pressure. The crude product was purified by silica gel chromatography eluting with a gradient of PE to 5% EA in PE. Fractions were concentrated to afford the title compound (830.00 mg, 4.34 mmol, 33.60% yield) as light yellow liquid. LC-MS: (ES, m/z): RT=1.010 min, LC-MS: m/z=190.9 [M+1]. ¹H NMR (400 MHz, Chloroform-d) 7.19 (s, 1H), 3.94 (s, 3H), 3.74 (s, 3H),

### Step 3: (3-methoxy-1-methyl-1H-pyrazol-5-yl)boronic acid

To a solution of 4-bromo-3-methoxy-1-methyl-1H-pyrazole (700 mg, 3.66 mmol, 1 eq) in THF (5 mL) was added n-BuLi (2.5 M, 1.76 mL, 1.2 eq) at −78° C. and stirred for 5 mins under N₂. 2-isopropoxy-4,4,5,5-tetramethyl-1,3,2-dioxaborolane (1.02 g, 5.50 mmol, 1.12 mL, 1.5 eq) was added to the reaction mixture at −78° C. The reaction was stirred at 25° C. for 30 mins. The reaction was diluted with EA (50 mL) and washed with water (50 mL×3). The organic layer was dried over Na₂SO₄, filtered and evaporated. The crude product was purified by prep-HPLC (column: Phenomenex luna C18 250*50 mm*10 um; mobile phase: [water (10 mM NH₄HCO₃)-ACN]; B %: 10%-40%, 20 min) to afford the title compound (1 g, crude) as yellow oil. LC-MS: (ES, m/z): RT=0.675 min, LC-MS: m/z=239.1 [M+1]. ¹H NMR (400 MHz, CD₃OD-d) δ 7.95 (s, 1H), 2.89 (s, 3H), 2.73 (s, 3H),

### Intermediate BP-28: 7-bromo-5-fluoroquinazolin-4(3H)-one

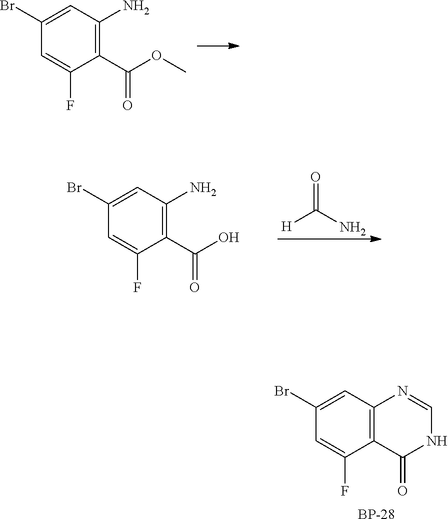

### Step 1: 2-amino-4-bromo-6-fluorobenzoic acid

To a solution of methyl 2-amino-4-bromo-6-fluorobenzoate (3 g, 12.09 mmol, 1 eq) in dioxane (20 mL) and H₂O (20 mL) was added LiOH·H₂O (5.08 g, 120.94 mmol, 10 eq). The mixture was stirred at rt for 2 hrs. The mixture was concentrated under reduced pressure. To the residue was added H₂O (10 mL), and HCl (1 M) was added to adjust to pH=6. The mixture was filtered and the solid was concentrated under reduced pressure to afford the title compound (2.5 g, 10.68 mmol, 88.33% yield) as a brown solid. LC-MS: (ES, m/z): RT=1.056 min, LC-MS: m/z=233.9 [M+1].

### Step 2: 7-bromo-5-fluoroquinazolin-4(3H)-one

A mixture of 2-amino-4-bromo-6-fluorobenzoic acid (2.5 g, 10.68 mmol, 1 eq) and formamide (12.03 g, 267.07 mmol, 10.65 mL, 25 eq) was heated to 170° C. for 3 hrs. The reaction mixture was quenched by addition of H₂O (25 mL) at 25° C. The mixture was filtered and the filtrate was concentrated under reduced pressure to afford the title compound (2 g, 8.23 mmol, 77.03% yield) as a brown solid. LC-MS: (ES, m/z): RT=0.914 min, LC-MS: m/z=242.9 [M+1].

### Intermediate BP-29: N4-(benzo[d]thiazol-6-yl)-7-methoxyquinazoline-4,6-diamine

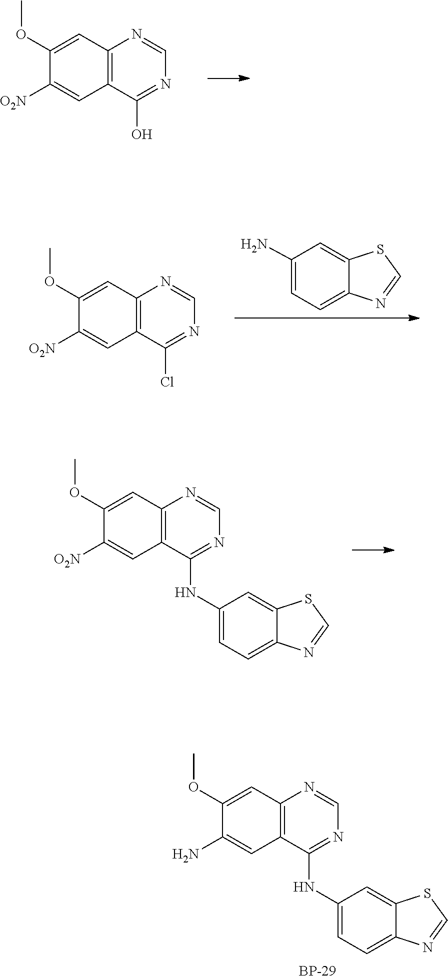 [SMILES: COc1cc2ncnc(O)c2cc1[N+](=O)[O-] ; COc1cc2ncnc(Cl)c2cc1[N+](=O)[O-] ; Nc1ccc2ncsc2c1 ; COc1cc2ncnc(Nc3ccc4ncsc4c3)c2cc1[N+](=O)[O-] ; COc1cc2ncnc(Nc3ccc4ncsc4c3)c2cc1N ; "BP-29"]

### Step 1: 4-chloro-7-methoxy-6-nitroquinazoline

To the mixture of 7-methoxy-6-nitroquinazolin-4-ol (400 mg, 1.81 mmol, 1 eq) in POCl₃ (7 mL) was added DIPEA (116.87 mg, 904.28 umol, 157.51 uL, 0.5 eq) at rt, the mixture was stirred at 100° C. for 3 hrs. The mixture was concentrated under vacuum to afford crude product. The crude product was purified by prep-TLC (PE:EA=1:1) to afford the title compound (450 mg, crude) as a yellow solid.

### Step 2: N-(7-methoxy-6-nitroquinazolin-4-yl)benzo[d]thiazol-6-amine

The mixture of 4-chloro-7-methoxy-6-nitroquinazoline (440 mg, 1.84 mmol, 1 eq) and benzo[d]thiazol-6-amine (413.72 mg, 2.75 mmol, 1.5 eq) in IPA (5 mL) was stirred at 90° C. for 3 hrs. The mixture was concentrated under vacuum. The crude product was added DMF (2 mL) and EA (5 mL), then filtered. The solid was collected to afford the title compound (200 mg, 566.00 umol, 30.82% yield) as a yellow solid. LC-MS: (ES, m/z): RT=1.387 min, LC-MS: m/z=354.1 [M+1].

### Step 3: N4-(benzo[d]thiazol-6-yl)-7-methoxyquinazoline-4,6-diamine

To a mixture of N-(7-methoxy-6-nitroquinazolin-4-yl)benzo[d]thiazol-6-amine (180 mg, 509.40 umol, 1 eq) in THF (5 mL), H₂O (1 mL) and DMF (2 mL) was added Fe (142.24 mg, 2.55 mmol, 5 eq) and NH₄Cl (136.24 mg, 2.55 mmol, 5 eq) at rt. The mixture was stirred at 70° C. for 1 hr. The reaction was filtered and the filtrate was concentrated under vacuum. The crude product was purified by prep-HPLC (column: Waters Xbridge Prep OBD C18 150*40 mm*10 um; mobile phase: [water (0.04% NH₃H₂O+10 mM NH₄HCO₃)-ACN]; B %: 5%-35%, 10 min) to afford the title compound (100 mg, 309.24 umol, 60.71% yield) as a white solid. LC-MS: (ES, m/z): RT=1.219 min, LC-MS: m/z=324.1 [M+1].

### Intermediate BP-30: N4-(benzo[d]thiazol-6-yl)-7-(1-methyl-1H-pyrazol-3-yl)-N6-(piperidin-4-yl)quinazoline-4,6-diamine

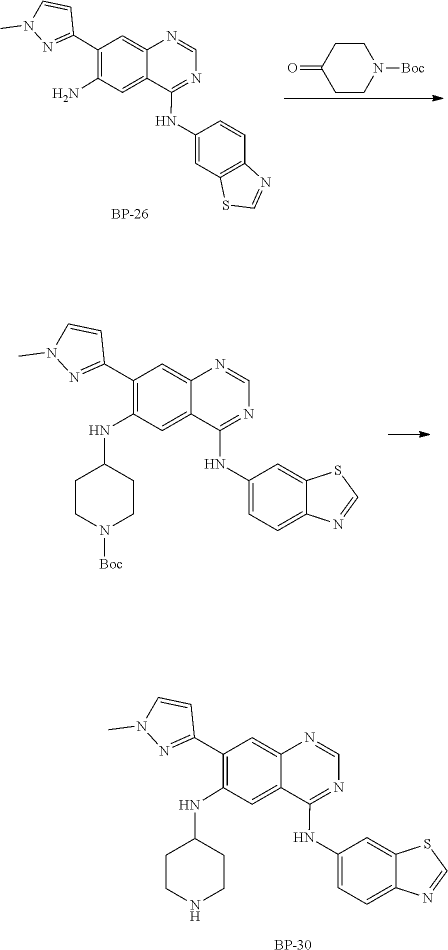 [SMILES: Cn1ccc(-c2cc3ncnc(Nc4ccc5ncsc5c4)c3cc2N)n1 ; CC(C)(C)OC(=O)N1CCC(=O)CC1 ; "BP-26" ; Cn1ccc(-c2cc3ncnc(Nc4ccc5ncsc5c4)c3cc2NC2CCN(C(=O)OC(C)(C)C)CC2)n1 ; Cn1ccc(-c2cc3ncnc(Nc4ccc5ncsc5c4)c3cc2NC2CCNCC2)n1 ; "BP-30"]

### Step 1: tert-butyl 4-((4-(benzo[d]thiazol-6-ylamino)-7-(1-methyl-1H-pyrazol-3-yl)quinazolin-6-yl)amino)piperidine-1-carboxylate

To a solution of Intermediate BP-26 (200 mg, 535.57 umol, 1 eq), tert-butyl 4-oxopiperidine-1-carboxylate (533.55 mg, 2.68 mmol, 5 eq) in DCM (20 mL) was added Ti(i-PrO)₄ (456.65 mg, 1.61 mmol, 474.20 uL, 3 eq). The reaction was stirred at 25° C. for 12 hrs. NaBH₄ (60.79 mg, 1.61 mmol, 3 eq) was added and the mixture was stirred at 25° C. for 2 hrs. The reaction mixture was quenched by addition MeOH (30 mL), the reaction mixture was stirred for 30 min. Then the reaction mixture was concentrated under reduced pressure to afford the title compound (200 mg, crude) was brown solid. LC-MS: (ES, m/z): RT=1.340 min, LC-MS: m/z=557.2 [M+1].

### Step 2: N4-(benzo[d]thiazol-6-yl)-7-(1-methyl-1H-pyrazol-3-yl)-N6-(piperidin-4-yl)quinazoline-4,6-diamine

A mixture of tert-butyl 4-((4-(benzo[d]thiazol-6-ylamino)-7-(1-methyl-1H-pyrazol-3-yl)quinazolin-6-yl)amino)piperidine-1-carboxylate (200 mg, 359.27 umol, 1 eq) in HCl/EA (10 mL) and EA (10 mL) was stirred at 25° C. for 1 hr. The mixture was concentrated under vacuum and the residue was purified by prep-HPLC (column: Phenomenex luna C18 250*50 mm*10 um; mobile phase: [water (0.05% HCl)—ACN]; B %: 5%-350, 20 min) to afford the title compound (10 mg, 21.90 umol, 6.10% yield) as a yellow solid. LC-MS: (ES, m/z): RT=0.987 min, LC-MS: m/z=457.2 [M+1].

### Intermediate BP-31: tert-butyl 4-(1H-pyrazol-3-yl)piperidine-1-carboxylate

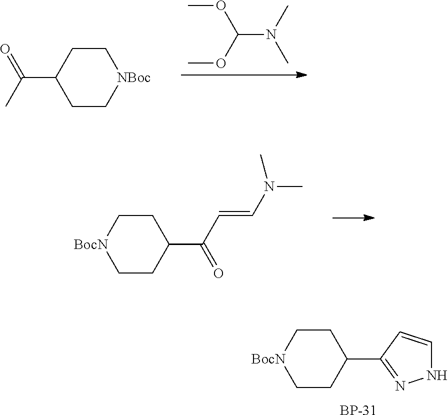

### Step 1: Synthesis of tert-butyl (E)-4-(3-(dimethylamino)acryloyl)piperidine-1-carboxylate

To a mixture of tert-butyl 4-acetylpiperidine-1-carboxylate (1 g, 4.39 mmol) in (dimethoxymethyl)dimethylamine (523 mg, 4.39 mmol) was stirred at 150° C. for 3 h. The mixture was concentrated under vacuum to give crude tert-butyl 4-(1H-pyrazol-3-yl)piperidine-1-carboxylate (900 mg, crude), which was used directly for next step. LC-MS: (ES, m/z): RT=0.671 min, LCMS: m/z=283 [M+1].

### Step 2: Synthesis of tert-butyl 4-(1H-pyrazol-3-yl)piperidine-1-carboxylate

To a solution of tert-butyl (E)-4-(3-(dimethylamino)acryloyl)piperidine-1-carboxylate (850 mg, 3.01 mmol) in EtOH (10 mL), was added hydrazine hydrate (180 mg, 3.61 mmol) and TEA (365 mg, 3.61 mmol) at 25° C. The reaction was stirred at 80° C. for 4 h. The mixture was extracted with EA (20 mL*3) and washed with brine (10 mL). The organic layer was dried with Na₂SO₄ and concentrated under vacuum. The residue was purified by Flash Column Silica-CS (DCM:MeOH=20:1 to 10:1) to afford 400 mg (52.9%) of the title compound as a yellow solid. LC-MS: (ES, m/z): RT=1.019 min, LCMS: m/z=196 [M−t-Bu+1],

### General Method for the Synthesis of N-Substituted Pyrazole Intermediates

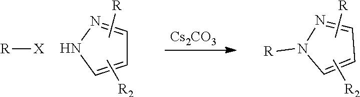

To a stirred solution of alkyl halide (1 eq) and (4,4,5,5,-tetramethyl-1,3,2-dioxaborolan-2-yl)-1H-pyrazole (1 eq) in DMF (0.2 mM) was added Cs₂CO₃ (2 eq). The mixture was stirred at 60° C. for 2 hours under N₂. The reaction mixture was extracted with DCM and washed by brine. The organic layers were concentrated under vacuum to afford the desired compound.

| | | | | LCMS |
|---|---|---|---|---|
| Int | | | | RT/Mass |
| # | Structure | Pyrazole | Alkyl Halide | (m/z) |
| BP-32 | 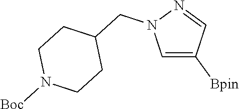 | 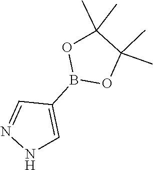 | 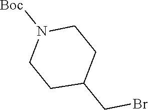 [SMILES: CC(C)(C)OC(=O)N1CCC(CBr)CC1] | 0.883 min/ 392 [M + 1] |
| BP-33 | 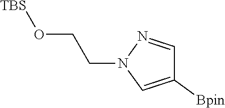 | 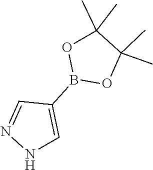 | 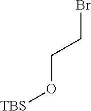 | 0.821 min/ 353 [M + 1] |
| BP-34 | 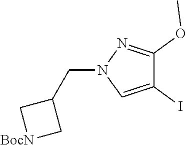 [SMILES: COc1nn(CC2CN(C(=O)OC(C)(C)C)C2)cc1I] | 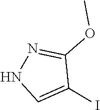 | 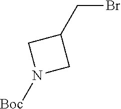 [SMILES: CC(C)(C)OC(=O)N1CC(CBr)C1] | 2.535 min/ 394 [M + 1] |
| BP-35 | 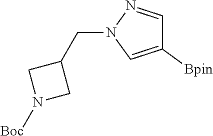 | 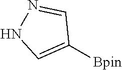 | 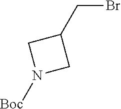 [SMILES: CC(C)(C)OC(=O)N1CC(CBr)C1] | 1.358 min/ 364 [M + 1] |
| BP-36 | 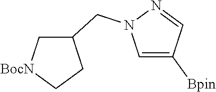 | 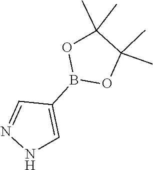 | 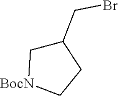 [SMILES: CC(C)(C)OC(=O)N1CCC(CBr)C1] | 1.490 min/ 378 [M + 1] |
| BP-37 | 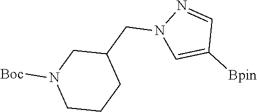 | 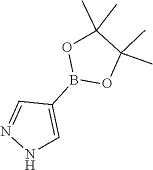 | 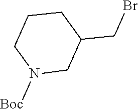 [SMILES: CC(C)(C)OC(=O)N1CCCC(CBr)C1] | 0.624 min/ 359 [M + 1] |

### Intermediate BP-40: cis-tert-butyl (3R,5S)-3-fluoro-5-hydroxypiperidine-1-carboxylate

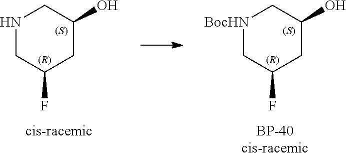

Di-tert-butyl dicarbonate (1.20 g, 5.52 mmol) was added to rac-(3S,5R)-5-fluoropiperidin-3-ol (330 mg, 2.76 mmol) and Na₂CO₃ (585 mg, 5.52 mmol) in dioxane/H₂O (10 mL) at rt. The resulting mixture was stirred at rt for 16 h. The mixture was diluted with EA 100 mL and washed with brine 50 mL*2. The organic layer was dried with Na₂SO₄ and concentrated under vacuum. The residue was purified by a silica gel column with PE:EA=10:1 to afford 400 mg of the title compound as a colorless oil. LC-MS: (ES, m/z): RT=0.671 min, LCMS: m/z=164 [M−57+1].

### Intermediate BP-41: trans-tert-butyl (3R,5R)-3-fluoro-5-hydroxypiperidine-1-carboxylate

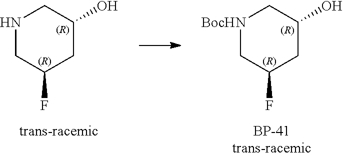

Di-tert-butyl dicarbonate (1.82 g, 8.38 mmol) was added to rac-(3R,5R)-5-fluoropiperidin-3-ol (500 mg, 4.19 mmol) and Na₂CO₃ (585 mg, 5.52 mmol) in dioxane/H₂O (10/2 mL) at rt. The resulting mixture was stirred at rt for 16 h. The mixture was diluted with EA 100 mL and washed with brine 50 mL*2, the organic layer was dried with Na₂SO₄ and concentrated under vacuum. The residue was purified by a silica gel column with PE:EA=10:1 to afford 600 mg of the title compound as a colorless oil. LC-MS: (ES, m/z): RT=0.890 min, LCMS: m/z=164 [M−57+1].

### Intermediate BP-42: 3-(dimethylamino)-1,1-difluoropropan-2-ol

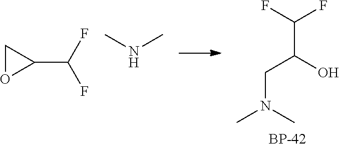

To a MW vial was add 2-(difluoromethyl)oxirane (35 mg, 0.372 mmol) in dimethylamine in 2M THF (192 μl, 0.384 mmol) in 1 ml THF. Sealed tube and heated to 50C ° C. 3 h and then at rt overnight. The material was taken to the next step crude.

### Intermediate BP-43: rac-trans-(1R,2R)-2-(dimethylamino)cyclobutan-1-ol

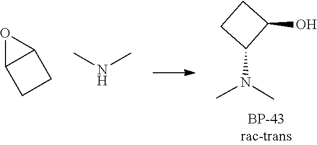

To a MW vial was add 5-oxabicyclo[2.1.0]pentane (125 mg, 1.783 mmol), 1,1,1,3,3,3-Hexafluoro-2-propanol (166 μl, 1.599 mmol) in dimethylamine (2000 μl, 4.00 mmol) in THF. The tube was sealed and heated to 100° C. for 4 hrs. TLC shows a new spot. The material was concentrated and taken to the next step.

### Intermediate BP-44: N-(6-iodo-7-methoxyquinazolin-4-yl)benzo[d]thiazol-6-amine

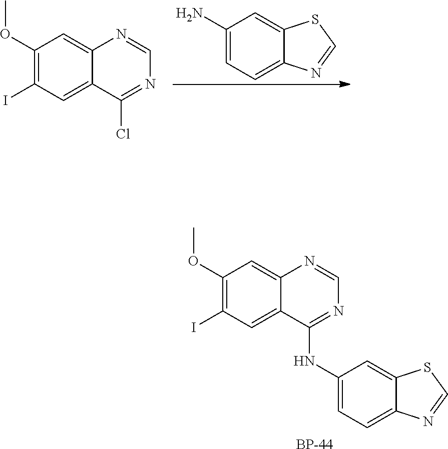 [SMILES: COc1cc2ncnc(Cl)c2cc1I ; Nc1ccc2ncsc2c1 ; COc1cc2ncnc(Nc3ccc4ncsc4c3)c2cc1I ; "BP-44"]

To a mixture of 4-chloro-6-iodo-7-methoxyquinazoline (500 mg, 1.56 mmol, 1.00 eq) in THF (10.00 mL) was added 1,3-benzothiazol-6-amine (468 mg, 3.12 mmol, 2.00 eq) and t-BuOK (349 mg, 3.12 mmol, 2.00 eq). The reaction mixture was stirred at 80° C. for 3 hr. The reaction mixture was concentrated under vacuum. The residue was purified by flash chromatography (5% MeOH in DCM) to afford the title compound (300 mg, yield: 44.3%) as a yellow solid. LC-MS: (ES, m/z): RT=1.202 min, LCMS: m/z=427 [M+1].

### Intermediate BP-45: 7-bromo-4-chloro-6-fluoroquinazoline

### Intermediate BP-45a: 7-bromo-6-fluoroquinazolin-4(3H)-one

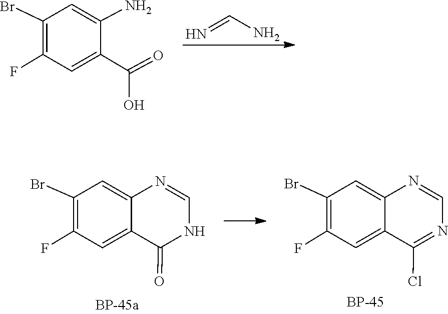

### Step 1: Synthesis of 7-bromo-6-fluoroquinazolin-4(3H)-one (Intermediate BP-45a)

A mixture of 2-amino-4-bromo-5-fluorobenzoic acid (464 mg, 2 mmol) and formimidamide (1.02 g, 10 mmol) in EtOH (5 mL) was refluxed for 10 h under N₂. 7-bromo-6-fluoroquinazolin-4(3H)-one was collected by filtration to afford the title compound (450 mg, yield: 94%) as a white solid LC-MS: (ES, m/z): RT=0.963 min, LCMS: m/z=243 245 [M+1]

### Step 2: Synthesis of 7-bromo-4-chloro-6-fluoroquinazoline

DIPEA (643 mg, 5 mmol), POCl3 (763 mg, 5 mmol), 7-bromo-6-fluoroquinazolin-4(3H)-one (243 mg, 1 mmol) were added into toluene (5 mL) in subsequence, then stirred at 100° C. for 10 h. The reaction was concentrated and the residue was purified by prep-TLC (PE/EA=1:1) to afford the title compound (150 mg, yield: 57%) as a white solid.

### Intermediate BP-46: Methyl 2-methyl-2-(4-(4,4,5,5-tetramethyl-1,3,2-dioxaborolan-2-yl)-1H-pyrazol-1-yl)propanoate

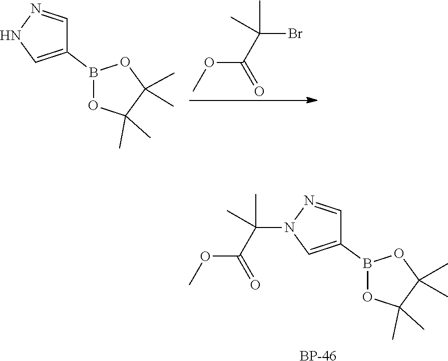

Cs₂CO₃ (1.67 g, 5.14 mmol) was added to 4-(4,4,5,5-tetramethyl-1,3,2-dioxaborolan-2-yl)-1H-pyrazole (500 mg, 2.57 mmol) and methyl 2-bromo-2-methylpropanoate (1.39 g, 7.71 mmol) in DMF (10 ml) at rt. The resulting mixture was heated to 60° C. for 16 h. The reaction mixture was diluted with EA (100 mL), and washed sequentially with water (100 mL*3) and saturated brine (100 ml). The organic layer was dried over Na₂SO₄, filtered and evaporated to afford crude product. The residue was purified by prep-TLC with DCM:MeOH=20:1 to afford the title compound (700 mg, 92.1%) as a light yellow solid. LC-MS: (ES, m/z): RT=0.822 min, LCMS: m/z=295 [M+1].

### Intermediate BP-47: methyl 2-(4-(4,4,5,5-tetramethyl-1,3,2-dioxaborolan-2-yl)-1H-pyrazol-1-yl)propanoate

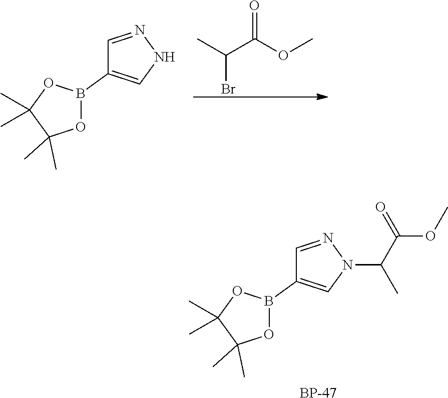

4-(4,4,5,5-tetramethyl-1,3,2-dioxaborolan-2-yl)-1H-pyrazole (500 mg, 2.57 mmol) and methyl 2-bromopropanoate (514 mg, 3.08 mmol) were added into DMF. Cs₂CO₃ (1.67 g, 5.14 mmol) was added at room temperature. The reaction was stirred at 100° C. for 3 hr. The reaction was extracted with EA and washed with brine. The organic layer was dried with Na₂SO₄ and concentrated to afford the title compound (550 mg, 76.4%) as a colorless oil.

### Intermediate BP-48: tert-butyl 3-(1-hydroxypropyl)azetidine-1-carboxylate

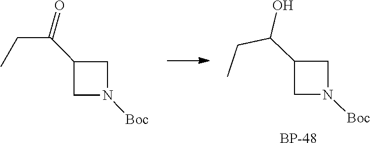

NaBH_{L}(355 mg, 9.36 mmol) was added to a solution of tert-butyl 3-propanoylazetidine-1-carboxylate (500 mg, 2.34 mmol) in MeOH, and stirred for 10 h. The reaction was quenched with saturated NH₄Cl, extracted with DCM, dried by Na₂SO₄ and concentrated as a oil (500 mg, crude).

### Intermediate BP-49: tert-butyl 5-hydroxy-2-oxa-7-azaspiro[3.5]nonane-7-carboxylate

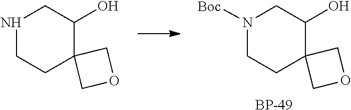

Di-tert-butyl dicarbonate (2.33 g, 10.7 mmol) was added batchwise to 2-oxa-7-azaspiro[3.5]nonan-5-ol (515 mg, 3.59 mmol) and Na₂CO₃ (1.13 g, 10.7 mmol) in dioxane/H₂O at rt. The mixture was stirred at rt for 16 h. The mixture was diluted with EA 100 mL and washed with brine 50 mL*2. The organic layer was dried with Na₂SO₄ and concentrated under vacuum. The residue was purified by silica gel column with PE:EA=2:1 to afford 700 mg of the title compound as a colorless oil. LC-MS: (ES, m/z): RT=0.841 min, m/z=188 [M−57+1].

### Intermediate BP-50: (R)-2-((4-(benzo[d]thiazol-6-ylamino)-7-(1-methyl-1H-pyrazol-4-yl)quinazolin-5-yl)oxy)propyl methanesulfonate

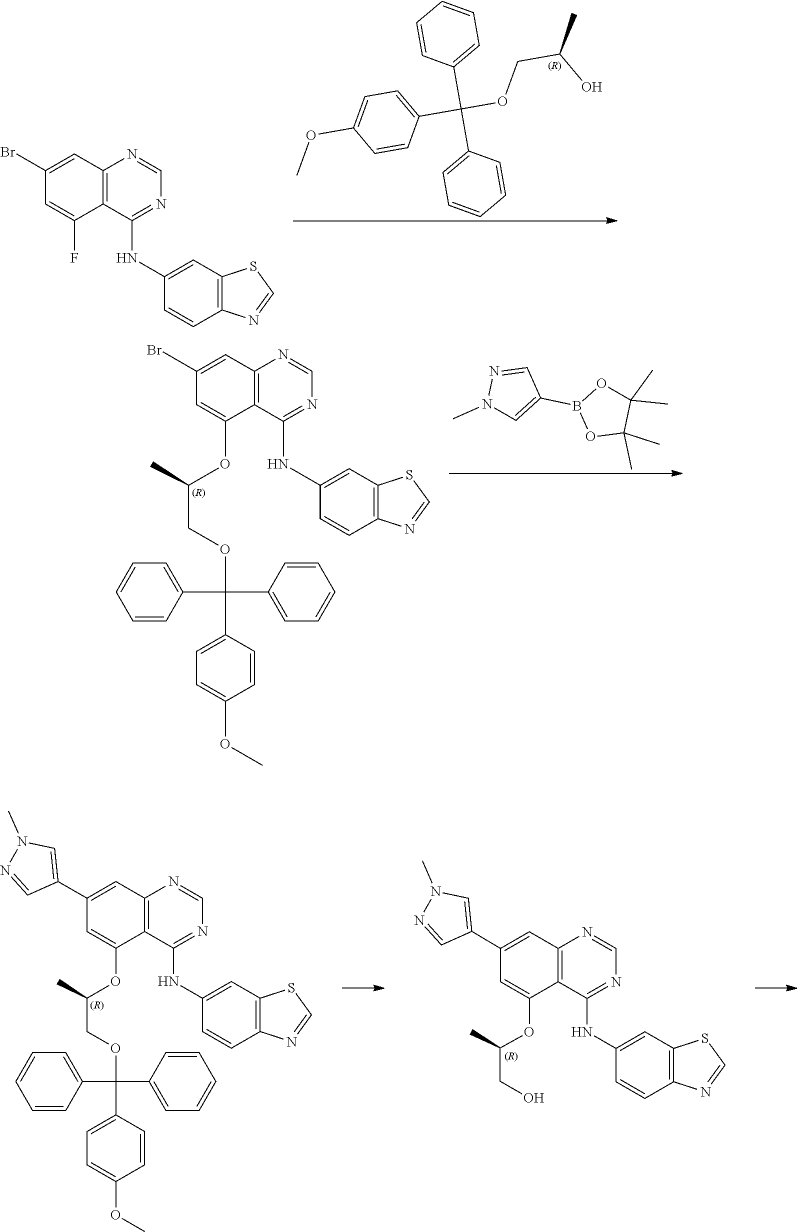

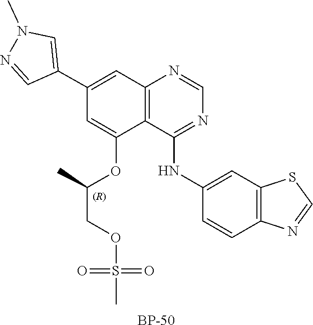

### Step 1: Synthesis of (R)—N-(7-bromo-5-((1-((4-methoxyphenyl)diphenylmethoxy)propan-2-yl)oxy)quinazolin-4-yl)benzo[d]thiazol-6-amine

To a mixture of (2R)-1-[(4-methoxyphenyl)diphenylmethoxy]propan-2-ol (1.48 g, 4.26 mmol, 2.0 eq) in DMF (20 mL) was added NaH (102 mg, 4.26 mmol, 2.00 eq) at 0° C. The reaction mixture was stirred at 0° C. for 15 min then Intermediate BP-59 was added to the reaction mixture. The reaction mixture was stirred at 120° C. for 16 hrs. The reaction mixture was added to ice water. The resulting solution was extracted with 2×30 mL of EA and the organic layers combined. The resulting mixture was washed with 20 mL of brine, dried over anhydrous sodium sulfate and concentrated under vacuum. The crude product was purified by column chromatography (EA in PE=0% to 70%) to afford the title compound (900 mg, 60.4%) as a yellow solid. LC-MS: (ES, m/z): RT=1.389 min, LCMS: m/z=703 [M+1].

### Step 2: (R)—N-(5-((1-((4-methoxyphenyl)diphenylmethoxy)propan-2-yl)oxy)-7-(1-methyl-1H-pyrazol-4-yl)quinazolin-4-yl)benzo[d]thiazol-6-amine

To a solution of (R)—N-(7-bromo-5-((1-((4-methoxyphenyl)diphenylmethoxy)propan-2-yl)oxy)quinazolin-4-yl)benzo[d]thiazol-6-amine (900 mg, 1.27 mmol, 1 eq) in 1,4-dioxane/H2O was added 1-methyl-4-(4,4,5,5-tetramethyl-1,3,2-dioxaborolan-2-yl)-1H-pyrazole (289 mg, 1.39 mmol, 1.1 eq), K₂CO₃ (350 mg, 2.54 mmol, 2 eq) and Pd(dppf)Cl₂ (92.8 mg, 127 umol, 0.1 eq) under nitrogen. The mixture was stirred at 80° C. for 4 hr. The reaction mixture was cooled to rt and the resulting solution was diluted with 25 mL of water. Extracted with ethyl acetate and the organic layers combined. The resulting mixture was washed with 20 mL of brine. The mixture was dried over anhydrous sodium sulfate and concentrated under vacuum. Purified by column chromatography (EA in PE=0% to 80%) to afford the title compound (700 mg, 78%) as a yellow solid. LC-MS: (ES, m/z): RT=1.200 min, LCMS: m/z=705 [M+1].

### Step 3: (R)-2-((4-(benzo[d]thiazol-6-ylamino)-7-(1-methyl-1H-pyrazol-4-yl)quinazolin-5-yl)oxy)propan-1-ol

The mixture of (R)—N-(5-((1-((4-methoxyphenyl)diphenylmethoxy)propan-2-yl)oxy)-7-(1-methyl-1H-pyrazol-4-yl)quinazolin-4-yl)benzo[d]thiazol-6-amine (700 mg, 0.9931 mmol, 1 eq) in TFA (3 mL) and DCM (10 mL) was stirred at 25° C. for 1 hr. LCMS showed the desired MS. The mixture was concentrated under vacuum. The residue was purified by silica gel column chromatography (DCM/EA=1:1) to afford the title compound (350 mg, 81%) as a yellow solid. LC-MS: (ES, m/z): RT=0.690 min, LCMS: m/z=433 [M+1].

### Step 4: Synthesis of (2R)-2-({4-[(1,3-benzothiazol-6-yl)amino]-7-(1-methyl-1H-pyrazol-4-yl)quinazolin-5-yl}oxy)propyl methanesulfonate

The mixture of (R)-2-((4-(benzo[d]thiazol-6-ylamino)-7-(1-methyl-1H-pyrazol-4-yl)quinazolin-5-yl)oxy)propan-1-ol (500 mg, 1.15 mmol, 1 eq) in THF (20 mL) was added TEA (464 mg, 4.6 mmol) and methanesulfonyl chloride (394 mg, 3.44 mmol) and the reaction mixture was stirred at 25° C. for 2 hours. The mixture was concentrated under vacuum. The resulting solution was diluted with 25 mL of water, extracted with 2×40 mL of ethyl acetate, and the organic layers combined. The resulting mixture was washed with 20 mL of brine, dried over anhydrous sodium sulfate and concentrated under vacuum to afford the title compound (500 mg, 85.1%) as a yellow solid. LC-MS: (ES, m/z): RT=0.998 min, LCMS: m/z=511 [M+1].

### Intermediate BP-51: N-(7-bromo-5-(((3S,4S)-4-methoxytetrahydrofuran-3-yl)oxy)quinazolin-4-yl)benzo[d]thiazol-6-amine

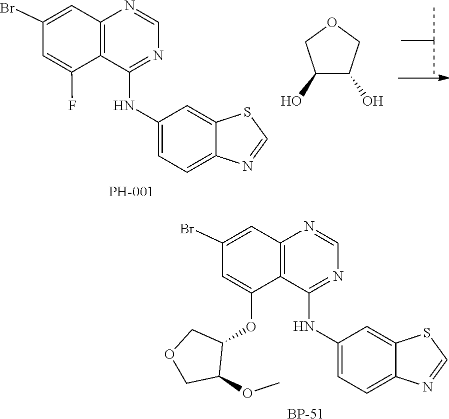

NaH (138 mg, 3.46 mmol 3 eq) was added to (3S,4S)-3,4-Dihydroxytetrahydrofuran (120 mg, 1.153 mmol) in DMF (3 ml) and stirred for 10 min. A solution of iodomethane, stabilized (164 mg, 1.153 mmol) in DMF (1 ml) was added slowly at rt. The reaction was stirred at rt for 1 hr. Intermediate BP-59 (215 mg, 0.573 mmol) was added to the reaction and heated to 100° C. for 1 hr. The reaction was cooled to rt and neutralized with 1N HCl. The solid was collected by filtration and dried to afford the title compound (291.7 mg, 0.616 mmol, 53.5% yield) as a beige color solid.

### Intermediate BP-52: 3-(dimethylamino)-1,1,1-trifluoropropan-2-ol

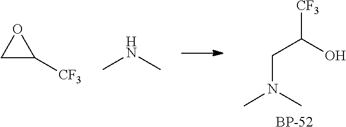

To a MW vial was add 2-(trifluoromethyl)oxirane (175 mg, 1.562 mmol), 1,1,1,3,3,3-Hexafluoro-2-propanol (175 mg, 1.041 mmol) in dimethylamine (2000 μl, 4.00 mmol) in THF. Sealed tube and heated to 100 C. Checked reaction by TLC after 4 hr. TLC shows a new spot. Therefore material is concentrated down on V10 and taken to the next step. 3-(dimethylamino)-1,1,1-trifluoropropan-2-ol (187.7 mg, 1.195 mmol, 76% yield).

### Intermediate BP-53: 4-bromo-2-(difluoromethyl)-1-methyl-1,2-dihydro-3H-pyrazol-3-one

### Intermediate BP-54: 4-bromo-3-(difluoromethoxy)-1-methyl-1H-pyrazole

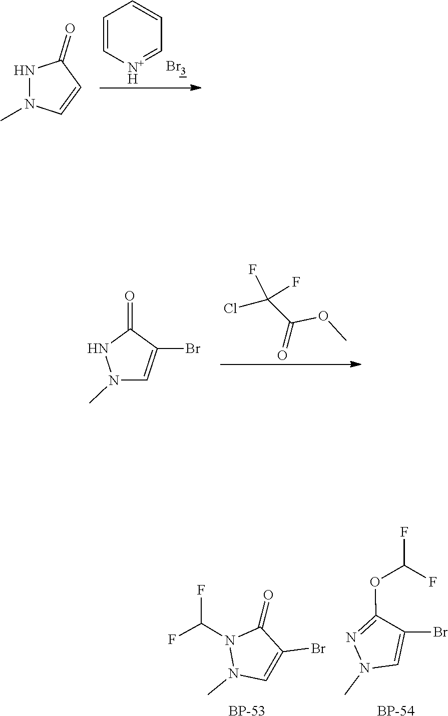

### Step 1: 4-bromo-1-methyl-1,2-dihydro-3H-pyrazol-3-one

To a solution of 1-methyl-2,3-dihydro-1H-pyrazol-3-one (10 g, 101 mmol) in MeOH was added Pyridinium bromide perbromide (32 g, 100 mmol). The reaction was stirred for 3 h then concentrated under vacuum. The residue was dissolved in 200 mL of water and extracted with EA. The organic layers were combined, washed with water and brine, dried over anhydrous sodium sulfate and concentrated to afford the title compound (12 g, 67% yield) as a yellow solid. LC-MS: (ES, m/z): RT=0.192 min, LCMS: m/z=177 [M+1], ¹H NMR (300 MHz, DMSO-d₆) δ 10.18 (s, 1H), 7.57 (s, 1H), 3.59 (s, 3H),

### Step 2: Synthesis of Intermediate BP-53: 4-bromo-2-(difluoromethyl)-1-methyl-1,2-dihydro-3H-pyrazol-3-one, and Intermediate BP-54: 4-bromo-3-(difluoromethoxy)-1-methyl-1H-pyrazole

To a solution of 4-bromo-1-methyl-2,3-dihydro-1H-pyrazol-3-one (3 g, 16.9 mmol) in DMF was added K₂CO₃ (7.21 mg, 52.3 mmol), the reaction was heated to 80° C. Ethyl 2-chloro-2,2-difluoroacetate (7.31 g, 50.6 mmol) was added and the solution was stirred for 3 h. The mixture was cooled to room temperature and the resulting solution was diluted with 100 mL of water. The solution was extracted with EA (2×80 mL). The organic layers were combined, washed with brine (80 mL of brine), dried over anhydrous sodium sulfate and concentrated under vacuum. The residue was purified by chromatography with PE:EA (10:1) to separate the two products. Appropriate fractions were combined and concentrated to afford:

First eluting compound: Intermediate BP-54 (1.2 g) as yellow oil: ¹H NMR (300 MHz, Methanol-d₄) δ 7.59 (s, 1H), 6.97 (t, J=73.2 Hz, 1H), 3.77 (s, 3H),

Second Eluting compound: Intermediate BP-53 (350 mg) as a white solid. ¹H-NMR: ¹H NMR (300 MHz, Methanol-d4) δ 8.02 (s, 1H), 7.34 (td, J=56.8, 0.7 Hz, 1H), 3.56 (d, J=0.9 Hz, 3H),

### Intermediate BP-55: tert-butyl (3S,4R)-3-fluoro-4-(4-(4,4,5,5-tetramethyl-1,3,2-dioxaborolan-2-yl)-1H-pyrazol-1-yl)piperidine-1-carboxylate

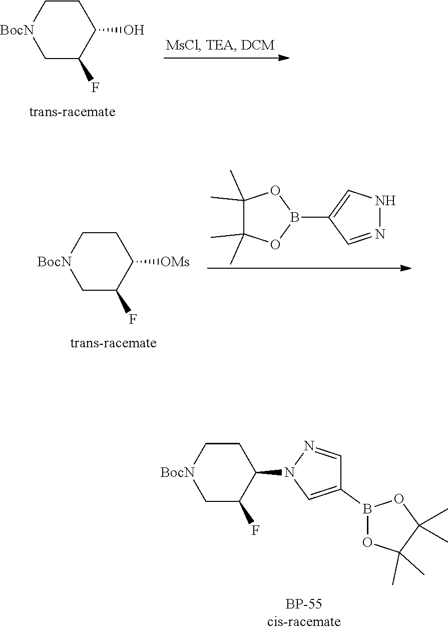 [SMILES: CC(C)(C)OC(=O)N1CC[C@H](O)[C@@H](F)C1 ; CC(C)(C)OC(=O)N1CC[C@H](OS(C)(=O)=O)[C@@H](F)C1 ; CC1(C)OB(c2cn[nH]c2)OC1(C)C ; CC(C)(C)OC(=O)N1CC[C@@H](n2cc(B3OC(C)(C)C(C)(C)O3)cn2)[C@@H](F)C1]

### Step 1: tert-butyl trans-(3S,4S)-3-fluoro-4-(methanesulfonyloxy)piperidine-1-carboxylate

To a solution of trans-tert-butyl (3S,4S)-3-fluoro-4-hydroxypiperidine-1-carboxylate (1 g, 4.56 mmol) and TEA (552 ng, 4.47 mmol) in DCM was added MsCl (571 mg, 5.01 mmol) at 0° C. wider N₂. The mixture was stirred for 2 hr at rt The mixture was diluted with water and extracted with EA. The organic layers were combined, dried over Na₂SO₄ and concentrated. The residue was purified by Prep-TLC with petroleum ether/ethyl acetate (10:1), to afford the title compound. (1.3 g (96.2%) as a light yellow oil. LC-MS: (ES, m/z): RT=0.759 min, LCMS: m/z=242 [M+1]

### Step 2: tert-butyl cis-(3S,4R)-3-fluoro-4-[4-(4,4,5,5-tetramethyl-1,3,2-dioxaborolan-2-yl)-1H-pyrazol-1-yl]piperidine-1-carboxylate

To a solution of tert-butyl trans-(3S,4S)-3-fluoro-4-(methanesulfonyloxy)piperidine-1-carboxylate (1.3 g, 4.37 mmol) and 4-(4,4,5,5-tetramethyl-1,3,2-dioxaborolan-2-yl)-LH-pyrazole (770 mg, 3.97 mmol) in DMF (15 mL) was added C₂CO₃ (1.81 g, 5.56 nmol) at rt. The mixture was stirred for 2 hirs at 90° C. The mixture was diluted with water and extracted with EA. The organic layers were combined, dried over Na₂SO₄ and concentrated. The residue was concentrated and the residue was purified by prep-TLC with petroleum ether/ethyl acetate (5:1) to afford the title compound (1.2 g, 76.9%) as a yellow solid. LC-MS: (ES, m/z): RT=1.358 min, LCMS: m/z=396 [M+1]

### Intermediate BP-56: tert-butyl (3S,4S)-3-fluoro-4-(4-(4,4,5,5-tetramethyl-1,3,2-dioxaborolan-2-yl)-1H-pyrazol-1-yl)piperidine-1-carboxylate

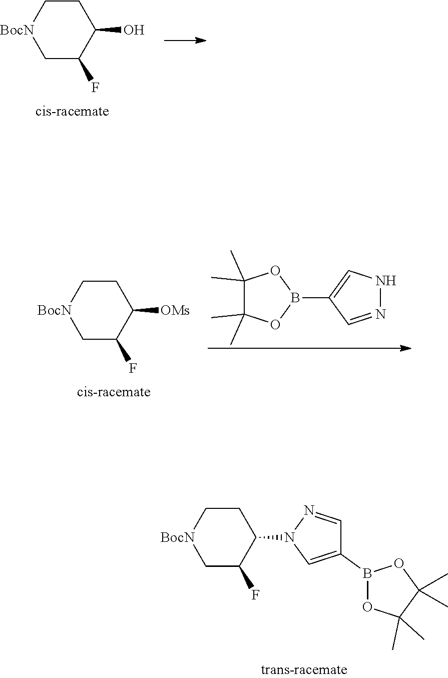

### Step 1: tert-butyl (3S,4R)-3-fluoro-4-((methylsulfonyl)oxy)piperidine-1-carboxylate

To a solution of tert-butyl cis-3-fluoro-4-hydroxypiperidine-1-carboxylate (1 g, 4.56 mmol), TEA (0.69 g, 6.84 mmol) in DCM (30 mL), was added MsCl (1 g, 8.73 mmol) at 0° C. then stirred at rt for 3 hrs. The reaction was quenched with water, extracted with EA. The organic layers were dried over Na₂SO₄, filtered and concentrated to afford the title compound (1.2 g, yield: 88%) as a light-yellow oil. LC-MS: (ES, m/z): RT=0.815 min, LCMS: m/z=320 [M+Na]

### Step 2: tert-butyl trans-3-fluoro-4-(4-(4,4,5,5-tetramethyl-1,3,2-dioxaborolan-2-yl)-1H-pyrazol-1-yl)piperidine-1-carboxylate

To a mixture of tert-butyl cis-3-fluoro-4-(methanesulfonyloxy)piperidine-1-carboxylate (900 mg, 3.03 mmol), 4-(4,4,5,5-tetrameth (588 mg, 3.03 mmol), Cs₂CO₃ (1.48 g, 4.54 mmol) in the DMF (15 mL) was stirred at 80° C. for 16 h. The reaction was concentrated and the residue was diluted with water. Extracted with EtOAc. The organic layer was dried over Na₂SO₄, filtered and concentrated to afford the title compound (700 mg, yield: 58.5%) as an off-white solid. LC-MS: (ES, m/z): RT=1.263 min, LCMS: m/z=396 [M+1]

### Intermediate BP-57: tert-butyl (3R,4R)-3-((4-(benzo[d]thiazol-6-ylamino)-7-(1-methyl-1H-pyrazol-4-yl)quinazolin-5-yl)oxy)-4-methoxypiperidine-1-carboxylate

### Intermediate BP-58: tert-butyl (3R,4R)-4-((4-(benzo[d]thiazol-6-ylamino)-7-(1-methyl-1H-pyrazol-4-yl)quinazolin-5-yl)oxy)-3-methoxypiperidine-1-carboxylate

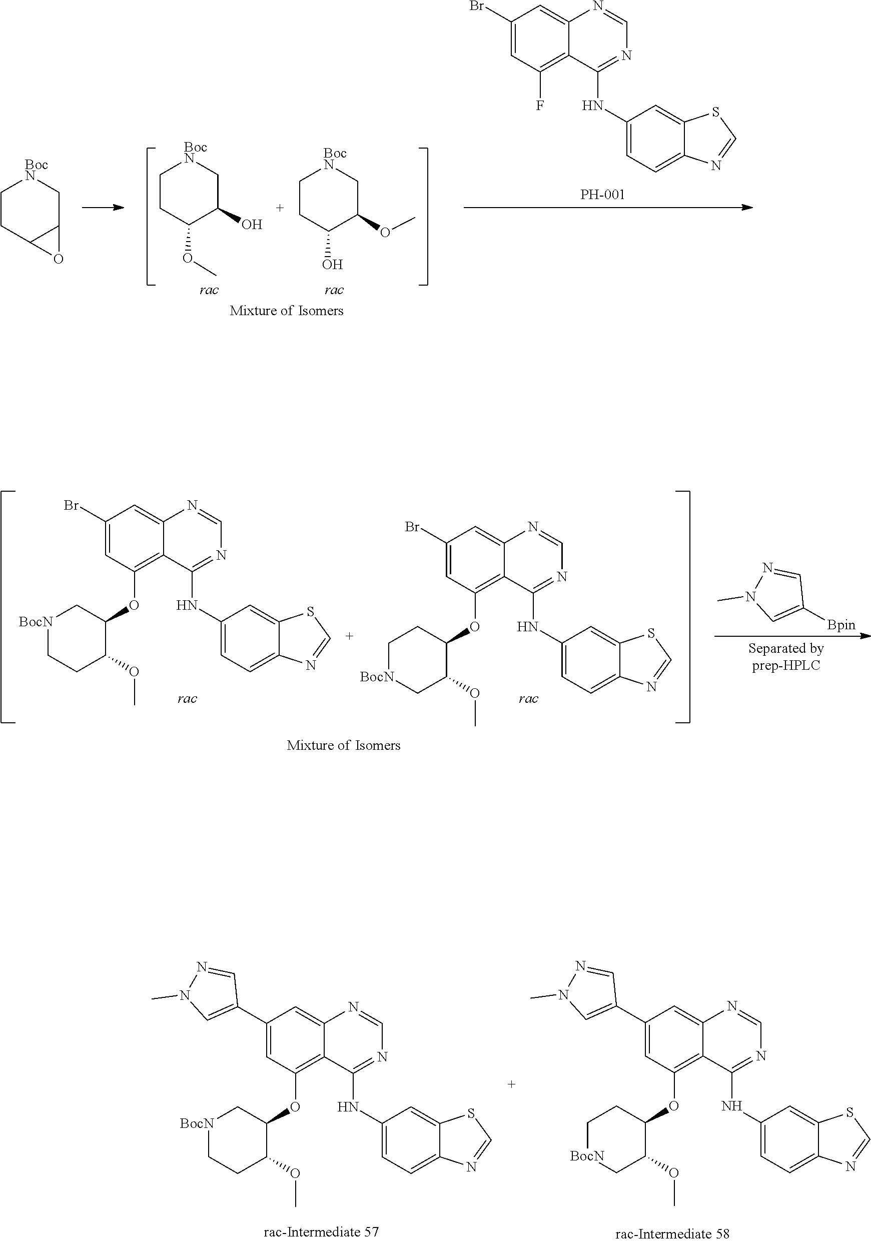

### Step 1: rac-tert-butyl (3R,4R)-3-hydroxy-4-methoxypiperidine-1-carboxylate and rac-tert-butyl (3R,4R)-4-hydroxy-3-methoxypiperidine-1-carboxylate

Into a 20-mL pressure tank reactor purged and maintained with an inert atmosphere of nitrogen, was placed tert-butyl 7-oxa-3-azabicyclo[4.1.0]heptane-3-carboxylate (500.00 mg, 2.509 mmol, 1.00 equiv), ACN (5.00 mL). MeOH (5.00 mL). The resuhing solution was stirred for 12 hr at room temperature. The reaction was concentrated under vacuum The residue was purified by silica gel column chromatography with DCM/MeOH (20/1) to afford the title compounds (400 mg, 68.92%) as a mixture.

### Step 2: rac-tert-butyl (3R,4R)-3-((4-(benzo[d]thiazol-6-ylamino)-7-bromoquinazolin-5-yl)oxy)-4-methoxypiperidine-1-carboxylate and rac-tert-butyl (3R,4R)-4-((4-(benzo[d]thiazol-6-ylamino)-7-bromoquinazolin-5-yl)oxy)-3-methoxypiperidine-1-carboxylate

Into a 20-mL pressure tank reactor purged and maintained with an inert atmosphere of nitrogen, was placed a mixture of rac-tert-butyl (3R,4R)-3-hydroxy-4-methoxypiperidine-1-carboxylate and tert-butyl (3R,4R)-4-hydroxy-3-methoxypiperidine-1-carboxylate (200.00 mg, 0.865 mmol, 1.00 equiv), DMF (5.00 mL), NaH (41.50 mg, 1.729 mmol, 2 equiv), N-(1,3-benzothiazol-6-yl)-7-bromo-5-fluoroquinazolin-4-amine (324.46 mg, 0.865 mmol, 1 equiv). The resulting solution was stirred for 3 hr at 80 degrees C. The reaction was then quenched by the addition of 5 mL of water. The solids were filtered out. The resulting solution was extracted with 3×10 mL of dichloromethane concentrated under vacuum. The residue was applied onto a silica gel column with DCM/MeOH (20/1) to afford the title compounds as a mixture (400 mg, 78.87%) as a light yellow solid. LC-MS: (ES, m/z): RT=1.193 min, LCMS: m/z=586,588 [M+1]

### Step 3: rac-tert-butyl (3R,4R)-3-((4-(benzo[d]thiazol-6-ylamino)-7-(1-methyl-1H-pyrazol-4-yl)quinazolin-5-yl)oxy)-4-methoxypiperidine-1-carboxylate and rac-tert-butyl (3R,4R)-4-((4-(benzo[d]thiazol-6-ylamino)-7-(1-methyl-1H-pyrazol-4-yl)quinazolin-5-yl)oxy)-3-methoxypiperidine-1-carboxylate

Into a 20-mL pressure tank reactor purged and maintained with an inert atmosphere of nitrogen, was placed mixture of rac-tert-butyl (3R,4R)-3-((4-(benzo[d]thiazol-6-ylamino)-7-bromoquinazolin-5-yl)oxy)-4-methoxypiperidine-1-carboxylate and rac-tert-butyl (3R,4R)-4-((4-(benzo[d]thiazol-6-ylamino)-7-bromoquinazolin-5-yl)oxy)-3-methoxypiperidine-1-carboxylate, Pd(dppf)Cl₂ (126.46 mg. 0.173 mmol, 0.1 equiv), K₂CO (477.48 mg, 3.46 mmol, 2 equiv), dioxane (5.00 mL), H₂O (1 ml) and 1-methyl-4-(4,4,5,5-tetramethyl-1,3,2-dioxaborolan-2-yl)-1H-pyrazole (396 mg, 1.90 mmol, 1.1 equiv). The resulting solution was stirred for 3 hr at 80° C. The reaction was quenched by the addition of 5 mL of water, extracted with 3×10 mL of dichloromethane and concentrated under vacuum. The residue was purified by silica gel column chromatography, eluting with DCM/MeOH (20/1) to afford the mixture od title compounds (750 mg) as a light yellow solid. LC-MS: (ES, m/z): RT=1.121 min, LCMS: m/z=588 [M+1]

### Separation of Isomers:

The mixture of isomers from step 3, (500 mg) was separated by prep-HPLC, Column: XBridge Prep OBD C18 Column, 19*250 mm, 5 μm; Mobile Phase A: Water (10 MMOL/L NH₄HCO₃), Mobile Phase B: ACN; Flow rate: 80 mL/mm. Gradient: 58% B to 58% B in 14 min, 58% B. Wave Length: 220 nm, to afford the title compounds:

Intermediate 57: rac-tert-butyl (3R,4R)-3-((4-(benzo[d]thiazol-6-ylamino)-7-(1-methyl-1H-pyrazol-4-yl)quinazolin-5-yl)oxy)-4-methoxypiperidine-1-carboxylate, (320 mg) as a white solid. LC-MS: (ES, m/z): RT=0.851 min, LCMS: m/z=588 [M+1]; ¹H NMR (400 MHz, DMSO-d₆) δ 10.12 (s, 1H), 9.33 (s, 1H), 8.84 (d, J=2.1 Hz, 1H), 8.50 (d, J=35.6 Hz, 2H), 8.21-8.08 (m, 2H), 7.81 (dd, J=8.9, 2.2 Hz, 1H), 7.59 (d, J=1.4 Hz, 1H), 7.45 (d, J=1.6 Hz, 1H), 4.98 (s, 1H), 4.10 (d, J=5.6 Hz, 1H), 3.92 (s, 3H), 3.76 (s, 2H), 3.34-3.15 (m, 1H), 3.30-2.96 (m, 1H), 2.29 (d, J=13.3 Hz, 1H), 1.86 (d, J=9.9 Hz, 1H), 1.43 (s, 9H),

Intermediate 58: rac-tert-butyl (3R,4R)-4-((4-(benzo[d]thiazol-6-ylamino)-7-(1-methyl-1H-pyrazol-4-yl)quinazolin-5-yl)oxy)-3-methoxypiperidine-1-carboxylate, (60 mg) as a white solid. LC-MS: (ES, m/z): RT=0.860 min, LCMS: m/z=588 [M+1]; ¹H NMR (400 MHz, DMSO-d₆) δ 9.83 (s, 1H), 9.31 (s, 1H), 8.66 (s, 1H), 8.47 (d, J=15.8 Hz, 2H), 8.16 (s, 1H), 8.10 (d, J=8.8 Hz, 1H), 7.73 (d, J=8.3 Hz, 1H), 7.58 (s, 1H), 7.50 (s, 1H), 5.05 (s, 1H), 4.86 (s, 1H), 4.51 (s, 1H), 4.28 (d, J=6.7 Hz, 1H), 3.90 (s, 3H), 1.83 (s, 2H), 1.28 (s, 2H), 1.18-0.63 (m, 9H),

### Intermediate BP-59: N-(7-bromo-5-fluoroquinazolin-4-yl)benzo[d]thiazol-6-amine

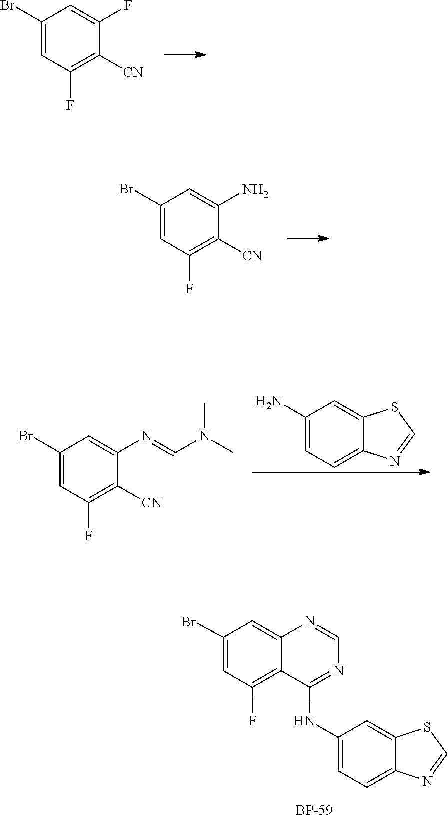 [SMILES: N#Cc1c(F)cc(Br)cc1F ; N#Cc1c(N)cc(Br)cc1F ; CN(C)/C=N/c1cc(Br)cc(F)c1C#N ; Nc1ccc2ncsc2c1 ; Fc1cc(Br)cc2ncnc(Nc3ccc4ncsc4c3)c12 ; "BP-59"]

### Step 1: 2-amino-4-bromo-6-fluorobenzonitrile

To a solution of 4-bromo-2,6-difluorobenzonitrile (10 g, 0.046 mol) in IPA (10 mL) was added amine hydrate (10 mL) at RT. The mixture was stirred for 2 hours at 90° C. The solvent was evaporated the crude product was used directly without further purification. LC-MS: (ES, m/z): RT=1.109 min, LCMS: m/z=213 [M−1].

### Step 2: N′-(5-bromo-2-cyano-3-fluorophenyl)-N,N-dimethylmethanimidamide

2-amino-4-bromo-6-fluorobenzonitrile (5 g, 0.023 mol) was added to DMF-DMA (50 mL) at RT. The mixture was stirred at 120° C. for 2 hours. After cooling to RT the solvent was evaporated and the residue taken up in diethyl ether, filtered and dried to obtain the title compound (4.9 g 79.0%). LC-MS: (ES, m/z): RT=1.218 min, LCMS: m/z=270 [M+1].

### Step 3: Synthesis of N-(1,3-benzothiazol-6-yl)-7-bromo-5-fluoroquinazolin-4-amine

N′-(5-bromo-2-cyano-3-fluorophenyl)-N,N-dimethylmethanimidamide (2.7 g, 9.99 mmol) and 1,3-benzothiazol-6-amine (1.63 g, 10.9 mmol) were dissolved in CH₃COOH. The mixture was stirred at 80° C. for 1 h. After cooling to RT, the reaction was added H2O. Filter and the filter cake was collected to obtain BP-59 (3.1 g, 82.8%). LC-MS: (ES, m/z): RT=1.036 min, LCMS: m/z=753 [M+1].

### Intermediate BP-60: N-(7-bromo-5-fluoroquinazolin-4-yl)-7-fluorobenzo[d]thiazol-6-amine

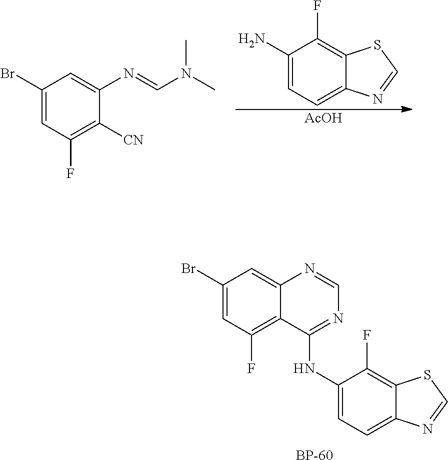 [SMILES: "AcOH" ; CN(C)/C=N/c1cc(Br)cc(F)c1C#N ; Nc1ccc2ncsc2c1F ; Fc1c(Nc2ncnc3cc(Br)cc(F)c23)ccc2ncsc12 ; "BP-60"]

### Step 1: Synthesis of 7-bromo-5-fluoro-N-(7-fluoro-1,3-benzothiazol-6-yl)quinazolin-4-amine

To a solution of N′-(5-bromo-2-cyano-3-fluorophenyl)-N,N-dimethylmethanimidamide (product of Intermediate BP-59, step 2) (200 mg, 740 umol) in AcOH was added 1,3-benzothiazol-6-amine (111 mg, 740 umol) at RT. The mixture was stirred for 1 h at 80° C. After cooling to RT the reaction mixture was diluted with water and the precipitate was filtered and washed with water. The crude product was used for the next step without further purification. LC-MS: (ES, m/z): RT=0.672 min, LCMS: m/z=393.0 [M+1].

### Intermediate BP-60 (Alternate Route): N-(7-bromo-5-fluoroquinazolin-4-yl)-7-fluorobenzo[d]thiazol-6-amine

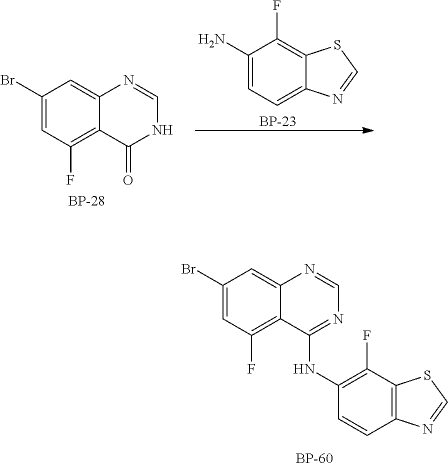

To a solution of Intermediate BP-28 (300 mg, 1.15 mmol, 1 eq) in DCE (3 mL) was added BP-23 (192.97 mg, 1.15 mmol, 1 eq) at rt, the reaction was stirred at 25° C. for 0.5 hr. The reaction mixture was filtered and the filter cake was concentrated in vacuum. The mixture was purified by prep-HPLC (column: Waters Xbridge Prep OBD C18 150*40 mm*10 um; mobile phase: [water (0.05% NH₃H₂O+10 mM NH₄HCO₃)-ACN]; B %: 30%-60%, 8 min) to afford the title compound (500 mg, crude) as a yellow solid. LC-MS: (ES, m/z): RT=2.126 min, LC-MS: m/z=394.9 [M+1].

### Intermediate BP-61: 4-[(1,3-benzothiazol-6-yl)amino]-7-bromoquinazolin-5-ol

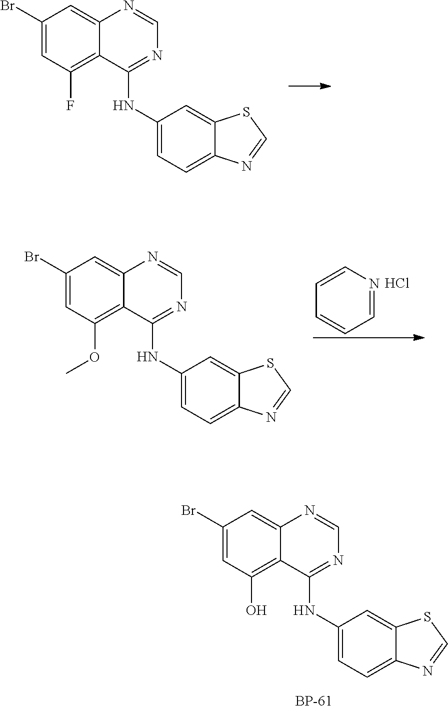 [SMILES: Fc1cc(Br)cc2ncnc(Nc3ccc4ncsc4c3)c12 ; COc1cc(Br)cc2ncnc(Nc3ccc4ncsc4c3)c12 ; c1ccncc1 ; Cl ; Oc1cc(Br)cc2ncnc(Nc3ccc4ncsc4c3)c12 ; "BP-61"]

### Step 1: Synthesis of N-(7-bromo-5-methoxyquinazolin-4-yl)benzo[d]thiazol-6-amine

To a stirred solution of Intermediate BP-59 (2 g, 5.3 mmol) in MeOH/1,4-dioxane (20 mL) was added Cs₂CO₃ (2.7 g, 10.6 mmol) under N₂. The mixture was stirred at 110° C. for 2 hours. After cooling to rt, the reaction was extracted with DCM and concentrated under vacuum to afford the title compound (1.5 g, 72%) as yellow solid.

### Step 2: Synthesis of 4-[(1,3-benzothiazol-6-yl)amino]-7-bromoquinazolin-5-ol

To a stirred solution of N-(7-bromo-5-methoxyquinazolin-4-yl)benzo[d]thiazol-6-amine (100 mg, 258 μmol) in Pyridine (2 mL) was added pyridine hydrochloride (147 mg, 1.28 mmol) under a nitrogen atmosphere. The resulting mixture was stirred at 110° C. for 2 hours. After cooling to rt, the reaction was filtered and the filter cake was collected. The crude was dissolved in MeOH and the pH was adjusted to pH=7.5 with TEA. Extracted with DCM and concentrated. To afford (80 mg, 83%) as yellow solid. LC-MS: (ES, m/z): RT=0.614 min, LCMS: m/z=373 [M+1].

### Intermediate BP-62: 4-(benzo[d]thiazol-6-ylamino)-7-(1-methyl-1H-pyrazol-4-yl)quinazolin-5-ol

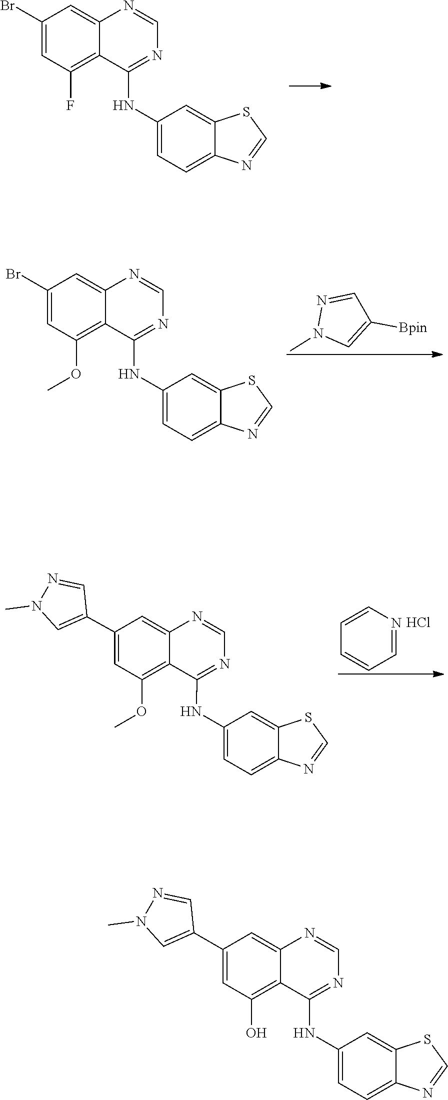

### Step 1: Synthesis of N-(1,3-benzothiazol-6-yl)-7-bromo-5-methoxyquinazolin-4-amine

To a solution of Intermediate BP-59 (2 g, 5.3 mmol) MeOH/1,4-dioxane (1:1, 20 mL) was added Cs₂CO₃ (2.7 g, 10.6 mmol) under N₂. The mixture was stirred at 110° C. for 2 hours. After cooling to RT, the reaction was extracted with DCM and concentrated under vacuum to afford the title compound (1.5 g, 72%) as yellow solid. LC-MS: (ES, m/z): RT=0.608 min, LCMS: m/z=387 [M+1].

### Step 2: Synthesis of N-(1,3-benzothiazol-6-yl)-5-methoxy-7-(1-methyl-1H-pyrazol-4-yl)quinazolin-4-amine

To a solution of N-(1,3-benzothiazol-6-yl)-7-bromo-5-methoxyquinazolin-4-amine (1.02 g, 2.64 mmol) and 1-methyl-4-(4,4,5,5-tetramethyl-1,3,2-dioxaborolan-2-yl)-1H-pyrazole (0.5 g, 2.40 mmol) in 1,4-dioxane/H₂O were added Pd(PPh₃)₄ (138 mg, 120 μmol) and K₂CO₃ (663 mg, 4.80 mmol) under N₂. The mixture was stirred at 100° C. for 2 hours. The reaction was extracted with DCM and purified by Prep-TLC (DCM/MeOH=10:1). Afforded the title compound (0.7 g, 70%) as white solid. LC-MS: (ES, m/z): RT=0.970 min, LCMS: m/z=389 [M+1].

### Step 3: Synthesis of 4-(benzo[d]thiazol-6-ylamino)-7-(1-methyl-1H-pyrazol-4-yl)quinazolin-5-ol

To a solution of N-(1,3-benzothiazol-6-yl)-5-methoxy-7-(1-methyl-1H-pyrazol-4-yl)quinazolin-4-amine (200 mg, 514 μmol) in pyridine (2 mL) was added pyridine hydrochloride (296 mg, 2.57 mmol) under N₂. The resulting mixture was stirred at 110° C. for 2 hours. After cooling to rt, the reaction was filtered and the filter cake was collected. The crude material was dissolved in MeOH, the pH adjusted to pH=7.5 with TEA and extracted with DCM to afford BP-62 (140 mg 73%) as yellow solid. LC-MS: (ES, m/z): RT=1.048 min, LCMS: m/z=375 [M+1].

### Intermediate BP-64: N-(1,3-benzothiazol-6-yl)-7-(3-methoxy-1-methyl-1H-pyrazol-4-yl)-5-{[1-(2-methoxyethyl)piperidin-4-yl]oxy}quinazolin-4-amine

### B-63: tert-butyl 4-((4-(benzo[d]thiazol-6-ylamino)-7-bromoquinazolin-5-yl)oxy)piperidine-1-carboxylate

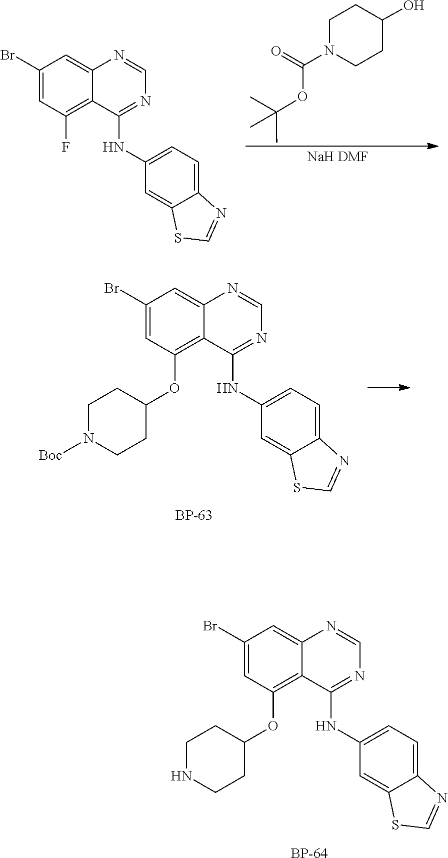 [SMILES: Fc1cc(Br)cc2ncnc(Nc3ccc4ncsc4c3)c12 ; CC(C)(C)OC(=O)N1CCC(O)CC1 ; CC(C)(C)OC(=O)N1CCC(Oc2cc(Br)cc3ncnc(Nc4ccc5ncsc5c4)c23)CC1 ; "BP-63" ; Brc1cc(OC2CCNCC2)c2c(Nc3ccc4ncsc4c3)ncnc2c1 ; "BP-64"]

### Step 1: Synthesis of tert-butyl 4-({4-[(1,3-benzothiazol-6-yl)amino]-7-bromoquinazolin-5-yl}oxy)piperidine-1-carboxylate

NaH (444 mg, 11.1 mmol) was added to tert-butyl 4-hydroxypiperidine-1-carboxylate (2.23 g, 11.1 mmol) in DMF (50 mL) at 0° C. After stirring for 30 min, Intermediate BP-59 (3.5 g, 9.32 mmol) was added and the mixture was stirred at 100° C. for 2 h. The reaction was then quenched by the addition of 100 mL of water. The resulting solution was extracted with EA and concentrated. The residue was purified by a silica gel column with DCM:MEOH=20:1. This afforded BP-63 (3.9 g, 75%) as an off-white solid. LC-MS: (ES, mi/z): RT=0.740 min, LCMS: mi/z=556 [M+1]

### Step 2: Synthesis of N-(1,3-benzothiazol-6-yl)-7-bromo-5-(piperidin-4-yloxy)quinazolin-4-amine

HCl (4 M) in dioxane (40 mL) was added to BP-64 (3.9 g, 7.00 mmol) in DCM (50 mL). The reaction was stirred at room temperature for 2 h. The mixture was concentrated under vacuum to afford BP-64 (2.2 g, 68%) as a yellow solid. LC-MS: (ES, m/z): RT=0.531 min, LCMS: m/z=456 [M+1]

### Intermediate BP-66: 7-bromo-N-(7-fluoro-1,3-benzothiazol-6-yl)-5-(piperidin-4-yloxy)quinazolin-4-amine

### Intermediate BP-65: tert-butyl 4-((7-bromo-4-((7-fluorobenzo[d]thiazol-6-yl)amino)quinazolin-5-yl)oxy)piperidine-1-carboxylate

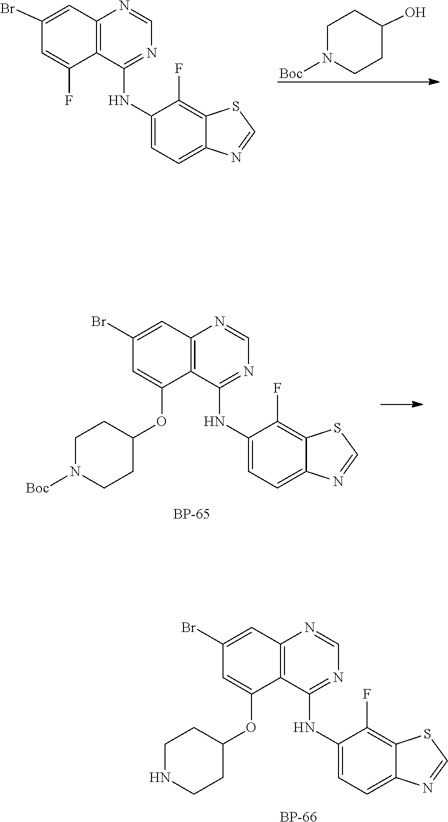 [SMILES: Fc1c(Nc2ncnc3cc(Br)cc(F)c23)ccc2ncsc12 ; CC(C)(C)OC(=O)N1CCC(O)CC1 ; CC(C)(C)OC(=O)N1CCC(Oc2cc(Br)cc3ncnc(Nc4ccc5ncsc5c4F)c23)CC1 ; "BP-65" ; Fc1c(Nc2ncnc3cc(Br)cc(OC4CCNCC4)c23)ccc2ncsc12 ; "BP-66"]

### Step 1: Synthesis of tert-butyl 4-(7-bromo-4-(7-fluorobenzo[d]thiazol-6-ylamino)quinazolin-5-yloxy)piperidine-1-carboxylate

To a solution of tert-butyl 4-hydroxypiperidine-1-carboxylate (764 mg, 3.80 mmol) in DMF (5 mL) was added NaH (151 mg, 3.80 mmol). The mixture was stirred at rt for 10 min. Intermediate BP-60 (750 mg, 1.90 mmol) was added and stirred at 80° C. for 2 h. The reaction was quenched with water and concentrated to dryness. The residue was purified on silica gel column with 10% MeOH in DCM to afford Intermediate BP-65 (850 mg, yield: 77.8%) as an off-white solid. LC-MS: (ES, m/z): RT=0.864 min, LCMS: m/z=574, 576 [M+1]

### Step 2: Synthesis of 7-bromo-N-(7-fluoro-1,3-benzothiazol-6-yl)-5-(piperidin-4-yloxy)quinazolin-4-amine

To a solution of Intermediate BP-65 (800 mg, 1.39 mmol) in Dioxane (10 mL) was added 7M HCl in Dioxane (10 mL). The reaction was stirred at r.t. for 1 h. Concentrated to dryness. The residue was diluted with sat. Na₂CO₃. Extracted with DCM. The organic layer was dried over Na₂SO₄. Filtered and concentrated to dryness to afford Intermediate BP-66 (560 mg, yield: 84.8%) as a grey solid. LC-MS: (ES, m/z): RT=0.733 min, LCMS: m/z=474, 476 [M+1]

### Intermediate BP-67: N-(1,3-benzothiazol-6-yl)-5-fluoro-7-(1-methyl-1H-pyrazol-4-yl)quinazolin-4-amine

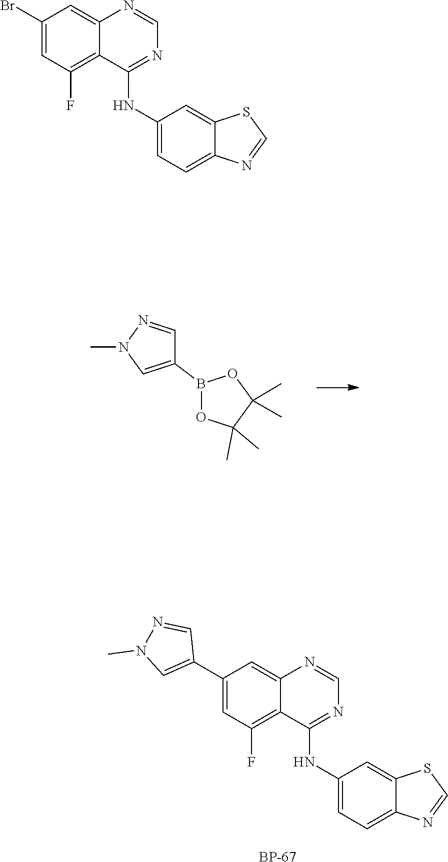

To a solution of Intermediate BP-59 in 1,4-dioxane/H2O was added 1-methyl-4-(4,4,5,5-tetramethyl-1,3,2-dioxaborolan-2-yl)-1H-pyrazole (3.03 g, 14.6 mmol, 1.10 eq), K₂CO₃ (3.67 g, 26.6 mmol, 2.00 eq), and Pd(PPh₃)₂Cl₂ (972 mg, 1.33 mmol, 0.10 eq) under nitrogen. The mixture was stirred at 80° C. for 4 hours. The reaction mixture was cooled to room temperature. The resulting solution was diluted with 50 mL of water. The resulting solution was extracted with 3×40 mL of ethyl acetate and the organic layers combined. The resulting mixture was washed with 50 mL of brine. The mixture was dried over anhydrous sodium sulfate and concentrated under vacuum. The product was purified by chromatography with PE:EA (1:1) to afford Intermediate BP-67 (4.5 g, 90.0%) as a yellow solid. LC-MS: (ES, m/z): RT=0.771 min, LCMS: m/z=377 [M+1]

### Intermediate BP-68: N-(7-(1-methyl-1H-pyrazol-4-yl)-5-(piperidin-4-yloxy)quinazolin-4-yl)benzo[d]thiazol-6-amine

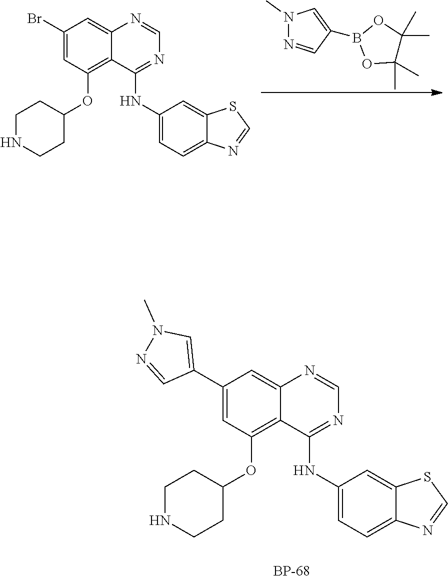

BP-64 (450 mg, 986 μmol) and 1-methyl-4-(4,4,5,5-tetramethyl-1,3,2-dioxaborolan-2-yl)-1H-pyrazole (245 mg, 1.18 mmol) in 1,4-dioxane (4 mL) and H₂O (1 mL) were added K₂CO₃ (271 mg, 1.97 mmol) and Pd(dppf)Cl₂ (35.8 mg, 49.3 μmol) under nitrogen atmosphere. The mixture was stirred at 100° C. for 2 hours. The reaction was extracted with DCM and concentrated under vacuum. The residue was purified by chromatography (5% MeOH in DCM) to give 400 mg (88%) of N-(1,3-benzothiazol-6-yl)-7-(1-methyl-1H-pyrazol-4-yl)-5-(piperidin-4-yloxy)quinazolin-4-amine as yellow solid. LC-MS: (ES, m/z): RT=0.873 min, LCMS: m/z=458 [M+1].

### Intermediate BP-69: N-(7-fluoro-1,3-benzothiazol-6-yl)-7-(1-methyl-1H-pyrazol-4-yl)-5-(piperidin-4-yloxy)quinazolin-4-amine

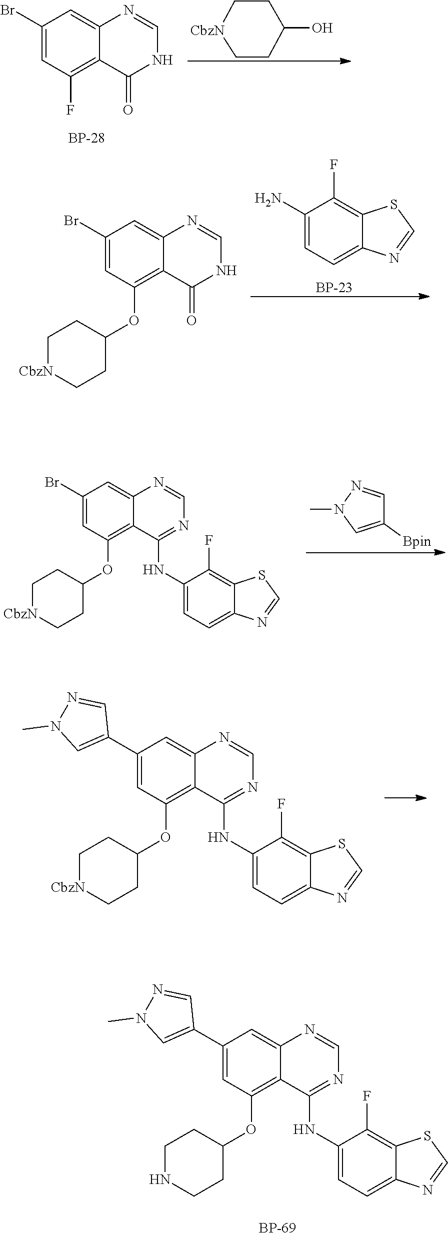

### Step 1: Synthesis of benzyl 4-[(7-bromo-4-oxo-3,4-dihydroquinazolin-5-yl)oxy]piperidine-1-carboxylate

To a mixture of benzyl 4-hydroxypiperidine-1-carboxylate (2.89 g, 12.3 mmol, 3.00 eq) in DMF (20.00 mL) was added NaH (329 mg, 8.22 mmol, 60% purity, 2.00 eq) at 0° C., the rection mixture was stirred at 0° C. for 15 min, then Intermediate BP-28 (1 g, 4.11 mmol, 1.00 eq) was added to the reaction mixture and stirred at 60° C. for 16 hrs. The reaction mixture was added to the ice water and the mixture was concentrated under vacuum. The residue was purified by a silica gel column with DCM:MeOH (20:1) to give 1.50 g (79.7%) of the title compound as a yellow solid. LC-MS: (ES, m/z): RT=1.272 min, LCMS: m/z=458 [M+1].

### Step 2: Synthesis of benzyl 4-((7-bromo-4-[(7-fluoro-1,3-benzothiazol-6-yl)amino]quinazolin-5-yl)oxy)piperidine-1-carboxylate

To a mixture of benzyl 4-[(7-bromo-4-oxo-3,4-dihydroquinazolin-5-yl)oxy]piperidine-1-carboxylate (300 mg, 0.6545 mmol, 1.00 eq) in DCE (8.00 mL) was added PPh₃ (513 mg, 1.960 mmol, 3.00 eq) and CCl₄ (5.99 mg, 3.92 mmol, 6.00 eq) at 25° C., the reaction mixture was stirred at 75° C. for 2 hrs. under N₂. A solution of Intermediate BP-23 (329 mg, 1.96 mmol, 3.00 eq) in DCE (2.00 mL) was added to the reaction mixture, and stirred at 25° C. for 2 hrs. under N₂, The mixture was concentrated under vacuum. The product was purified by a silica gel column with DCM:MeOH (20:1) to give 240 mg (60.4%) of the title compound as an off-white solid. LC-MS: (ES, m/z): RT=2.080 min, LCMS: m/z=608 [M+1].

### Step 3: benzyl 4-((4-[(7-fluoro-1,3-benzothiazol-6-yl)amino]-7-(1-methyl-1H-pyrazol-4-yl)quinazolin-5-yl)oxy)piperidine-1-carboxylate

To a solution of benzyl 4-((7-bromo-4-[(7-fluoro-1,3-benzothiazol-6-yl)amino]quinazolin-5-yl)oxy)piperidine-1-carboxylate (500 mg, 0.8217 mmol) in DMSO/H2O (10 mL/2.5 mL) was added 1-methyl-4-(4,4,5,5-tetramethyl-1,3,2-dioxaborolan-2-yl)-1H-pyrazole (187 mg, 903 μmol, 1.10 eq) was added K₂CO₃ (226 mg, 1.64 mmol, 2.00 eq) and XphosPd G2 (64.5 mg, 82.1 μmol, 0.10 eq). The resulting solution was stirred for 3 hour at 80° C. The reaction mixture was added to the ice water and the resulting solution was extracted with 3×30 mL of ethyl acetate and the organic layers combined. The resulting mixture was washed with 20 mL of brine. The mixture was dried over anhydrous sodium sulfate and concentrated under vacuum. The product was purified by prep-TLC with DCM:MeOH (15:1) to afford the title compound (400 mg, yield: 80.0%) as a white solid. LC-MS: (ES, m/z): RT=1.135 min, LCMS: m/z=610 [M+1]

### Step 4: N-(7-fluoro-1,3-benzothiazol-6-yl)-7-(1-methyl-1H-pyrazol-4-yl)-5-(piperidin-4-yloxy)quinazolin-4-amine

The mixture of benzyl 4-({4-[(7-fluoro-1,3-benzothiazol-6-yl)amino]-7-(1-methyl-1H-pyrazol-4-yl)quinazolin-5-yl}oxy)piperidine-1-carboxylate (400 mg, 0.6560 mmol, 1.00 eq) in DCM (10.00 mL) was added TMSI (656 mg, 3.28 mmol, 5.00 eq) 25° C. for 2 hr. The mixture was concentrated under vacuum. The product was purified by chromatography with DCM:MeOH, (10:1) to afford the title compound (260 mg, yield: 83.6%) as a yellow solid. LC-MS: (ES, m/z): RT=0.912 min, LCMS: m/z=476 [M+1]

### Intermediate BP-70a: Synthesis of (R)—N-(7-bromo-5-(1-(oxetan-3-yl)ethoxy)quinazolin-4-yl)benzo[d]thiazol-6-amine

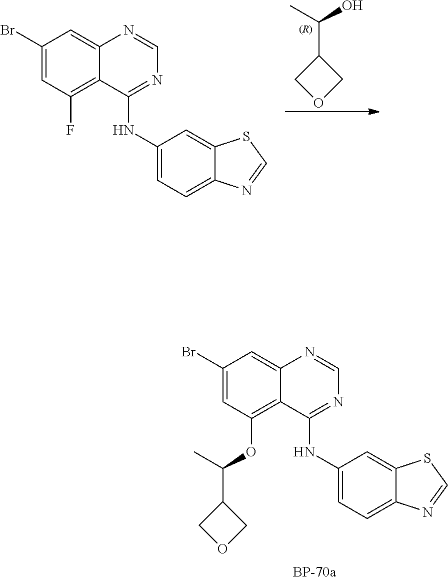

t-BUOK (19.6 g, 175 mmol) was added to Intermediate BP-59 (3.6 g, 9.59 mmol) and (1R)-1-(oxetan-3-yl)ethan-1-ol (1.95 g, 19.1 mmol) in THF at rt. The mixture was stirred at 80 degrees for 16 h The mixture was diluted with EA 200 mL and washed with brine 50 mL*2, the organic layer was dried with Na₂SO₄ and concentrated under vacuum. The residue was purified by a silica gel column with DCM:MeOH=15:1 to afford 2.35 g of the title compound as a white solid.

### Intermediate BP-70 (R)—N-(7-bromo-5-(1-(oxetan-3-yl)ethoxy)quinazolin-4-yl)-7-fluorobenzo[d]thiazol-6-amine

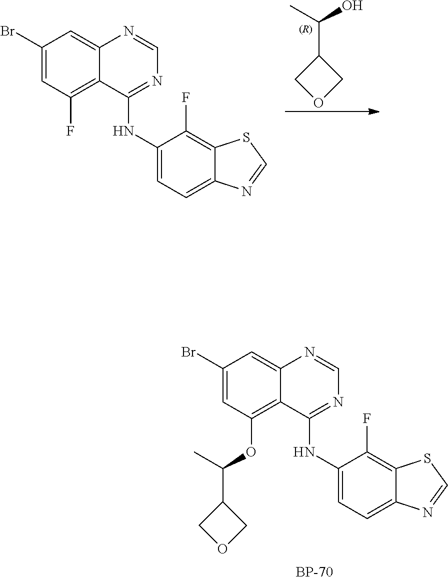

Intermediate BP-70 was prepared using the method of Intermediate BP-70a substituting BP-60 for BP-59 to afford the title compound.

### Intermediate BP-71: (R)—N-(7-bromo-5-((1-(dimethylamino)propan-2-yl)oxy)quinazolin-4-yl)benzo[d]thiazol-6-amine

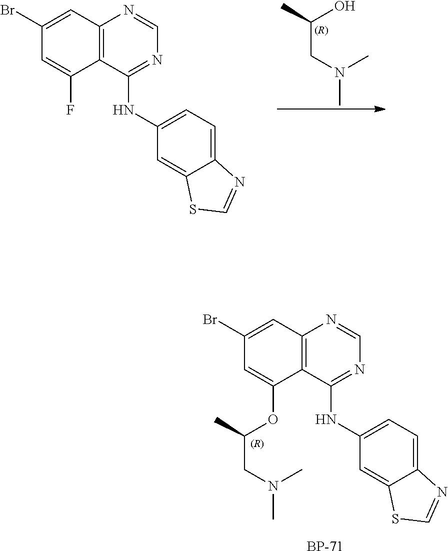

NaH (212 mg, 5.32 mmol) was added to (2R)-1-(dimethylamino)propan-2-ol (548 mg, 5.32 mmol) in THF at 0° C. for 20 minute. Intermediate BP-59 (1 g, 2.66 mmol) was added and the mixture was stirred for 60° C. for 3 h. The reaction mixture was diluted with EA (100 mL), and washed sequentially with water (100 mL*3) and saturated brine (100 mL*1). The organic layer was dried over Na₂SO₄, filtered and evaporated to afford crude product. The crude product was purified by a silica gel column with DCM:MeOH=20:1. This resulted in 1.1 g (90.9%) as a light yellow solid. LC-MS: (ES, m/z): RT=1.246 min, LCMS: m/z=458 [M+1],

### Intermediate BP-72: 7-bromo-5-{[(2R)-1-(dimethylamino)propan-2-yl]oxy}-N-(7-fluoro-1,3-benzothiazol-6-yl)quinazolin-4-amine

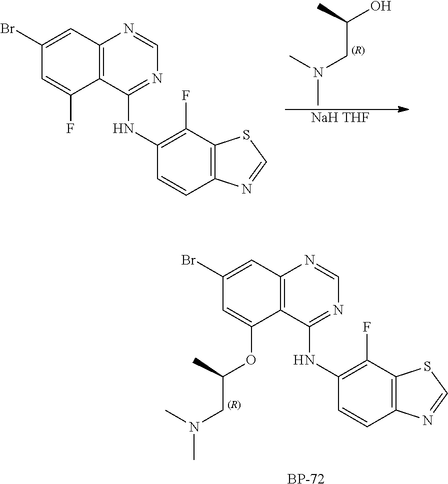

NaH (36.5 mg, 914 umol) was added to (2R)-1-(dimethylamino)propan-2-ol (393 mg, 3.81 mmol) in THF (10 mL) at 0° C. After stirring for 30 min, Intermediate BP-60 (300 mg, 762 μmol) was added and the mixture was heated to 60° C. for 2 h. The reaction was then quenched by the addition of 10 mL of water. The resulting solution was extracted with EA and concentrated. The crude product was purified by a prep-TLC with (DCM:MeOH=25:1) To afford Intermediate BP-72 300 mg (82%) as a yellow solid. LC-MS: (ES, m/z): RT=0.492 min, LCMS: m/z=476 [M+1]

### Intermediate BP-73: N-(5-((1-methylpiperidin-4-yl)oxy)-7-(4,4,5,5-tetramethyl-1,3,2-dioxaborolan-2-yl)quinazolin-4-yl)benzo[d]thiazol-6-amine

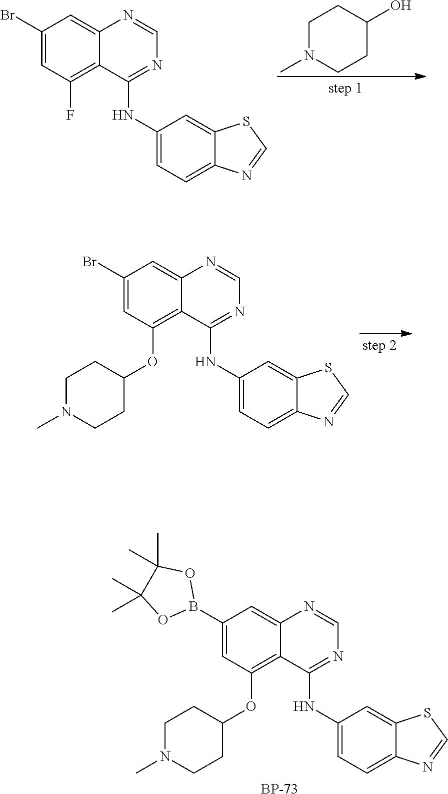

### Step 1: Synthesis of N-(1,3-benzothiazol-6-yl)-7-bromo-5-[(1-methylpiperidin-4-yl)oxy]quinazolin-4-amine

To a mixture of 1-methylpiperidin-4-ol (1.46 g, 12.7 mmol, 3.00 eq) in DMF (40 mL) was added NaH (285 mg, 11.9 mmol, 2.80 eq) at 0° C., the reaction mixture was stirred at 0° C. for 15 min, then N-(1,3-benzothiazol-6-yl)-7-bromo-5-fluoroquinazolin-4-amine (1.6 g, 4.26 mmol, 1.00 eq) was added to the reaction mixture, the reaction mixture was stirred at 80° C. for 16 hrs. The reaction mixture was added to the ice water and extracted with EA. The organic phase was concentrated under vacuum. The residue was purified by flash chromatography (10% MeOH in DCM) to give N-(1,3-benzothiazol-6-yl)-7-bromo-5-[(1-methylpiperidin-4-yl)oxy]quinazolin-4-amine (1.8 g, yield: 90.0%) as a off-white solid. LC-MS: (ES, m/z): RT=0.875 min, LCMS: m/z=470 [M+1]

### Step 2: Synthesis of N-(1,3-benzothiazol-6-yl)-5-[(1-methylpiperidin-4-yl)oxy]-7-(4,4,5,5-tetramethyl-1,3,2-dioxaborolan-2-yl)quinazolin-4-amine

To a solution of N-(1,3-benzothiazol-6-yl)-7-bromo-5-[(1-methylpiperidin-4-yl)oxy]quinazolin-4-amine (1.9 g, 4.03 mmol, 1.00 eq) in DMSO was added 4,4,5,5-tetramethyl-2-(4,4,5,5-tetramethyl-1,3,2-dioxaborolan-2-yl)-1,3,2-dioxaborolane (2.04 g, 8.06 mmol, 2.00 eq), KOAc (789 mg, 8.06 mmol, 2.00 eq) and Pd(dppf)Cl₂ (294 mg, 403 umol, 0.10 eq) under nitrogen. The mixture was stirred at 100° C. for 4 hours. LCMS showed the reaction was completed. The reaction mixture was cooled to room temperature. The mixture was concentrated under vacuum. The product was purified by chromatography with DCM:MeOH (10:1). This resulted in 1.10 g (52.8%) BP-73 as a yellow solid. LC-MS: (ES, m/z): RT=0.629 min, LCMS: m/z=436 [M+1]

### Synthesis Methods

### Method A1: Nucleophilic Addition Using NaH:

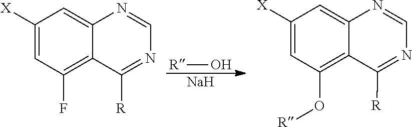

To a solution alcohol (R″—OH) (1-4 eq) in a polar aprotic solvent (typically DMF, but DMA, THF were also used) was added NaH (1.5-4 eq). The mixture was stirred at 0° C.-rt for 10-30 min. The fluoro-starting material (1 eq) was added and the reaction was stirred at 60° C.-100° C. for 2-24 hours as was needed. The reaction mixture was quenched with water and concentrated to dryness. Alternately the reaction was diluted with water and extracted with EA or DCM, washed with water and brine, dried over Na₂SO₄, filtered and concentrated. The residue was purified by prep-TLC, silica column chromatography or prep-HPLC as was appropriate for the desired product.

Method A2: Nucleophilic Addition Using Cs₂CO₃ as Base:

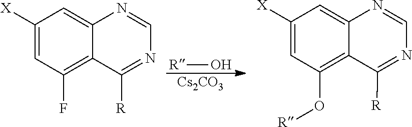

Into a reaction tube was added The fluoro-starting material (1 eq) alcohol (3 eq), Cs₂CO₃ (1.2-2 eq) in dioxane (unless noted) at rt. The mixture was stirred at 100° C. for 16 hours. The resulting mixture was concentrated under vacuum. The residue was purified by prep-TLC or silica column chromatography (normal phase solvent system: DCM & 5-20% MeOH)

Method A3: Nucleophilic Addition Using t-BuOK as Base

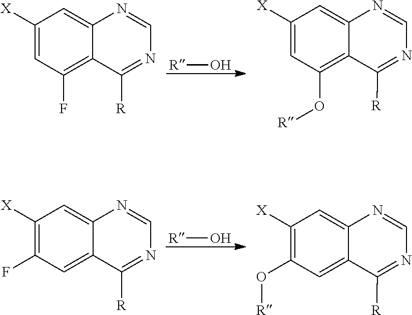

To a solution alcohol (R″—OH) (1-4 eq), the fluoro-starting material (1 eq) in THF was added t-BuOK, at rt. The reaction was stirred at 70° C.-100° C. for 2-16 hours as needed. The reaction mixture was quenched with water. If the desired product became a solid of suitable purity it was collected by filtration. If not, then the mixture was extracted with EA, washed with water and brine, dried and concentrated. The residue was purified by prep-TLC, silica column chromatography (normal phase solvent system: DCM & 5-20% MeOH) or prep-HPLC as was appropriate for the desired product.

### Method A3b: Nucleophilic Addition Using KHMDS as Base

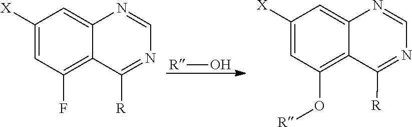

To a vial was added the alcohol (1 eq), DMA, 0.5M KHMDS in Toluene (1.2 eq) and stirred at rt for 15 min. Then solid aryl-fluoride (1.2 eq) was added and the reaction stirred at 70° C. The reaction was cooled to RT, diluted with EA (~1.5 reaction volumes) and 2-3 drops of TEA were added. A solid was collected by filtration to afford the desired intermediate which was taken to the next step. If the solid was of insufficient purity the material was also purified by prep-HPLC

### Method A4: Alkylation

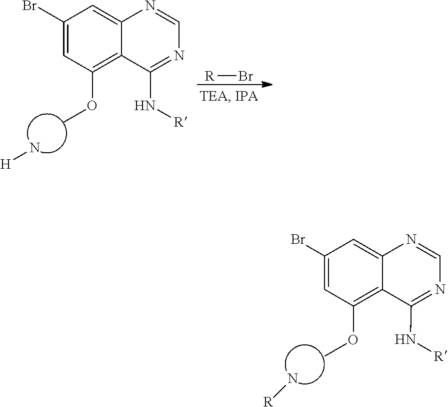

TEA (1-4 eq) was added to a solution of amine starting material (1 eq), alkyl halide (1.2-5 eq) and IPA at rt. The reaction was heated to 80-100° C. for 2-16 h. The reaction mixture was diluted with EA, washed with water and brine saturated brine. The organic layer was dried over Na₂SO₄, filtered and evaporated to afford crude product. The residue was purified by prep-TLC (DCM/MeOH system), silica column chromatography, (DCM/MeOH system) or prep-HPLC as was appropriate for the desired product

### Method A4b: Alkylation

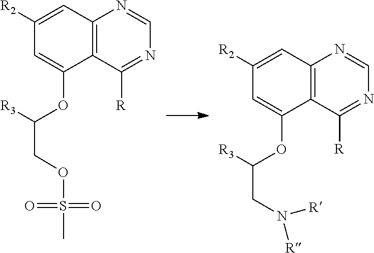

To a solution of the methane sulfonate (1 eq) in ACN (8 ml) was added an amine (5-10 eq) and K₂CO₃ (3-5 eq). The mixture was stirred at 80° C. overnight. The reaction was diluted with water and extracted with ethyl acetate. The organic layers were combined, washed with brine, dried over anhydrous sodium sulfate and concentrated under vacuum. The crude product was purified by Prep-HPLC. Appropriate fractions were concentrated to afford the desired product.

### Method A5: Alkylation

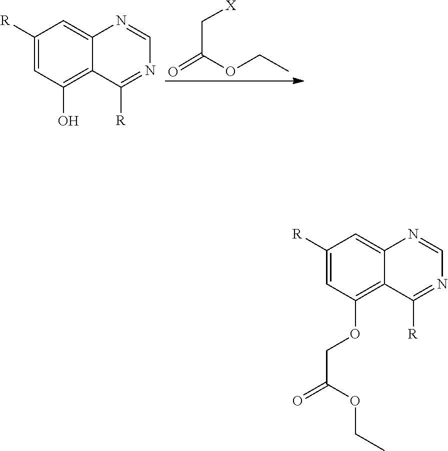

To a sealed tube was added: the hydroxy starting material (1 eq), alkyl bromide (1 eq), Cs₂CO₃ (1 eq) and DMF (0.16 mM). The reaction was heated at 60° C. for 12 h. The reaction was diluted with water and extracted with ethyl acetate. The organic layers were combined, washed with brine, dried over anhydrous sodium sulfate and concentrated under vacuum. The residue was purified by column chromatography (DCM:MeOH) to afford the desired product.

### Method B1: Suzuki Coupling (Pd(Dppf)Cl₂)

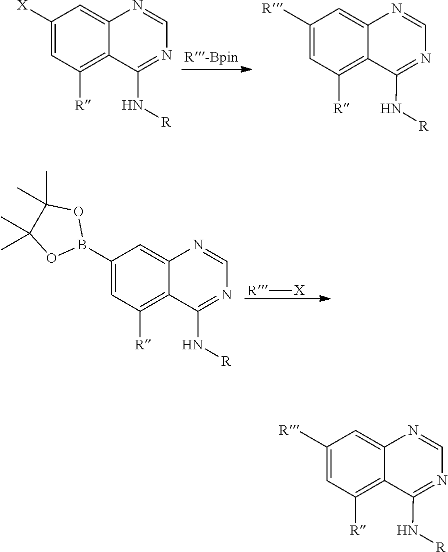

A mixture of aryl-halide (1 eq), boronate or boronate ester (1-3 eq), Pd(dppf)Cl₂ or Pd(dppf)Cl₂-DCM (10 mol %), K₂CO₃ or K₃PO₄ (1-3 eq) and dioxane (alternately DMF or DMA): water, (3:1-10:1), was stirred at between 70-100° C. for 2-24 hours. The mixture was typically extracted with DCM or EA then washed with water and brine. The organic layer was dried over Na2CO3 and concentrated to dryness. The residue was purified by prep-TLC (DCM/MeOH system), silica column chromatography, (DCM/MeOH system) or prep-HPLC as was appropriate for the desired product

### Method B1b: Suzuki Coupling (X-Phos)

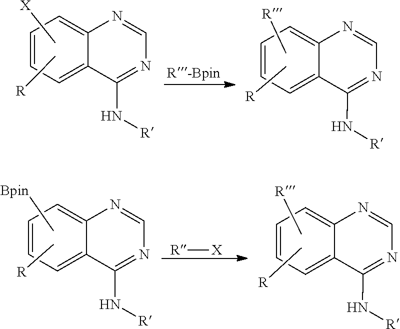

To a solution of aryl halide (110 mg, 0.2432 mmol, 1.00 eq) in a mixture of a polar aprotic solvent, typically DMSO (dioxane was also used) and water was added the appropriate boronate ester or boronic acid (1-1.5 eq), K₂CO₃ or K₃PO₄ (2 eq) and Xphos Pd G3 (1-10 mol %) under a nitrogen atmosphere. The mixture was stirred at 60° C.-100° C. for 2-24 hours as was needed. The reaction was cooled to room temperature, diluted with water and extracted with EA or DCM. The organic layers were combined, washed with brine, dried over anhydrous sodium sulfate and concentrated. The crude product was purified by Prep-HPLC to afford the desired compound.

### Method B2: Suzuki Coupling (Tetrakis)

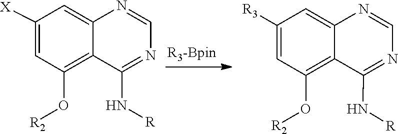

To a solution of aryl halide (1 eq) and the appropriate boronate ester (1.1-2 eq) or boronic acid in 1,4-dioxane/H₂O was added Pd(PPh₃)₄ (0.1-0.2 eq) and Base (1-5 eq, K₂CO₃, K₃PO₄, Cs₂CO₃) under N₂. The resulting mixture was stirred at 100° C. for 2-3 hours. The reaction was extracted with EA or DCM and concentrated. The residue was purified by Prep-HPLC to afford the desired compound.

### Method B3: ROCK-Phos Alcohol:

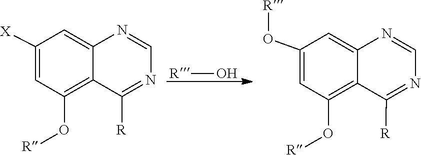

To a vial was added: RockPhosPdG3 (0.1 eq), Cs₂CO₃ (1.5-2 eq), aryl halide (1 eq), alcohol (5-10 eq) and dioxane at rt. The resulting mixture was heated 60-100° C. for 4-24 hrs. The reaction mixture was diluted with an EA and washed with water and brine. The organic layer was dried over Na₂SO₄, filtered and concentrated. The residue was purified by prep-TLC, silica column chromatography or prep-HPLC as was appropriate for the desired product.

Method B5a: Buchwald with BINAP

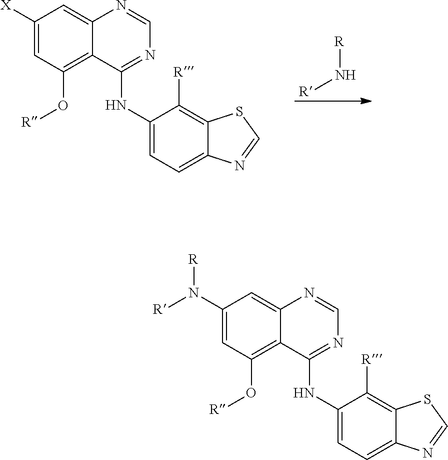

To a reaction vial was added: BINAP Pd G2 (0.1 eq), amine (1-3 eq), aryl halide (1 eq) and Cs₂CO₃ (1-3 eq) in dioxane at rt. The resulting mixture was stirred at 80-100° C. for 2-16 hr. The reaction mixture was diluted with water, extracted with EA, washed with water and brine. The organic layer was dried over Na₂SO₄, filtered and concentrated. The residue was purified by prep-TLC, silica column chromatography or prep-HPLC as was appropriate for the desired product.

Method B5b: Buchwald with RuPhos

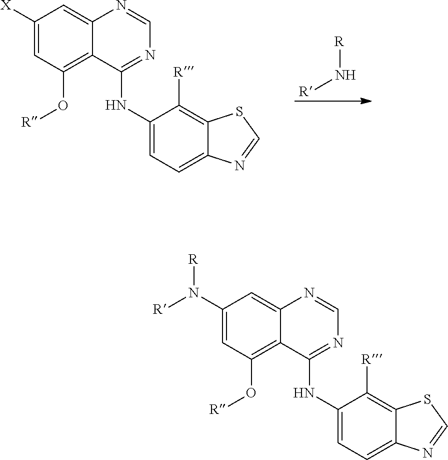

RuPhos Pd G₃ (5-10 mol %), Cs₂CO₃ (1-3 eq) was added the aryl halide (1 eq) and amine (1-5 eq) in dioxane at rt. The resulting mixture was heated to 80-100° C. for 12-16 hr. The reaction mixture was cooled to rt and diluted with EA. The mixture was washed sequentially with water and saturated brine. The organic layer was dried over Na₂SO₄, filtered and evaporated. The residue was purified by prep-TLC, silica column chromatography or prep-HPLC as was appropriate to afford the desired product.

### Method B5c: Buchwald RuPhos/BINAP

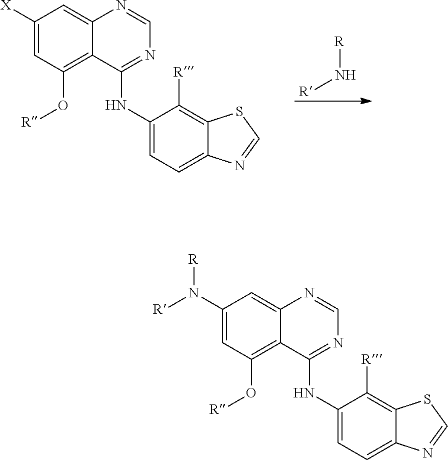

To a reaction vessel was added Bromide (1 eq), amine (1-2 eq), Sodium t-butoxide (2.5-3 eq), 2,2′-bis(diphenylphosphaneyl)-1,1′-binaphthalene (5-7 mol %), Tris(dibenzylideneacetone)dipalladium(0) (3-5 mol %) and RuPhos G3 (5 mol %). The reaction was typically run in DMA, however DMF was also used. The reaction was sparged with nitrogen and heated the reaction to 100° C. for 1-16 hrs. The residue was purified by silica column chromatography or prep-HPLC as was appropriate to afford the desired product.

Method B5d: Coupling XantPhos tBuONa

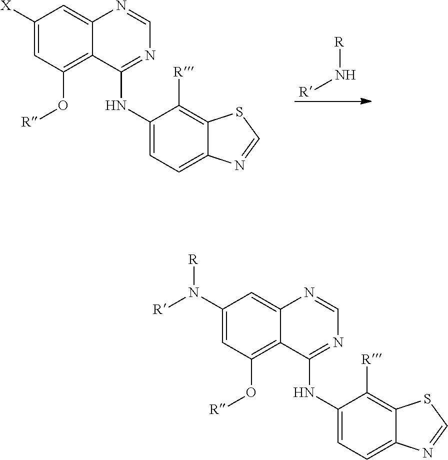

To a vial was added: Aryl halide (1 eq), aza coupling partner (1.4 eq), XantPhos Pd G3 (5 mol %), DMA and sodium t-Butoxide (2 eq). The reaction was sparged with nitrogen then heated at 100-120° C. for 3 hr-3 days. The reaction was filtered and the solution purified by prep-HPLC.

Method B5e: Suzuki XantPhos K₃PO₄

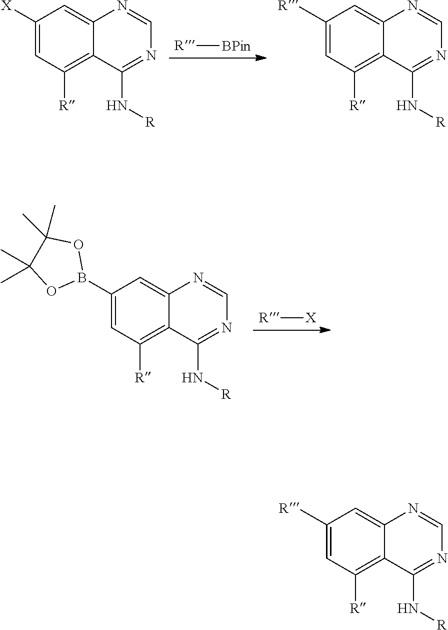

To a vial was added: Xantphos Pd G3 (0.05 eq), boronate ester or boronic acid (1-2 eq), aryl bromide (1 eq). DMF and aqueous 2M potassium phosphate (2.5-5 eq). The reaction was evacuated, purged with nitrogen and heated at 90-100° C. for 2-16 hours. The reaction was filtered and purified by prep-HPLC to afford the title compound.

Method B5f: N-Pyrazole coupling with BretPhos

To a reaction vessel was added: Cs₂CO₃ (1.5 eq), the aryl halide (1 eq), BrettPhos Pd G₃ (0.1 eq), the pyrazole (1.2 eq) and dioxane at rt. The reaction was stirred at 100° C. for 3 h. The mixture was diluted with EA, washed with water and brine. The organic layer was dried with Na₂SO₄ and concentrated under vacuum. The residue was purified by Prep-HPLC to afford the desired compound.

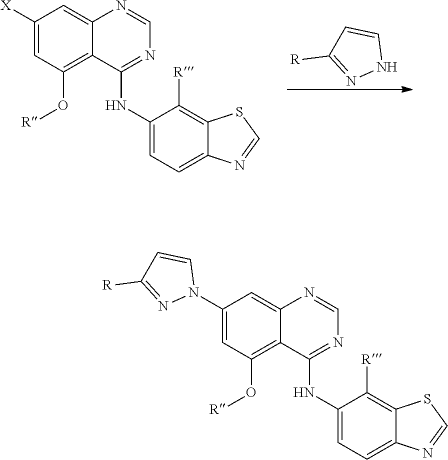

### Method B5 g: Buchwald Using E-Phos

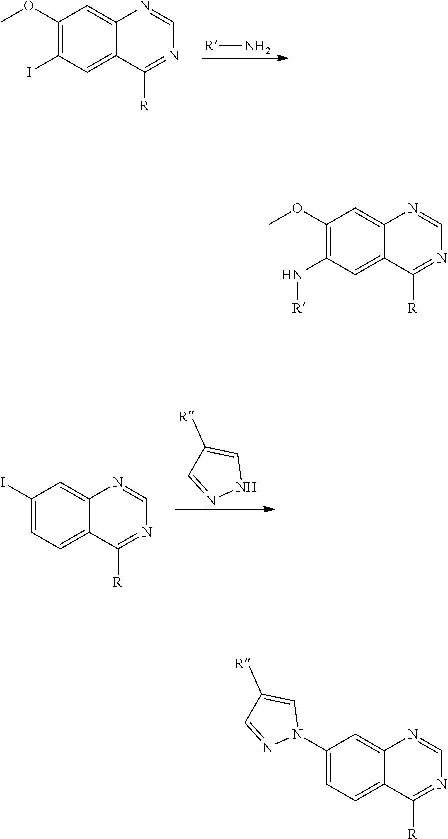

Aryl-hailide (1 equiv), the appropriate amine or pyrazole (1.1-2 equiv), EPhosPd G4 (0.05-0.1 equiv), EPhos (0.13 equiv) and base (NaOtBu or K₂CO₃, 2-3 equiv) and dioxane or toluene were combined under nitrogen. The mixture was heated at 80-100° C. for 3-18 h. After cooling to rt, the mixture was concentrated. The residue was purified by silica gel column chromatography (DCM/MeOH=15:1) to the desired product.

Method B5 h: XantPhos Pd G3, BINAP, Pd₂dba₃

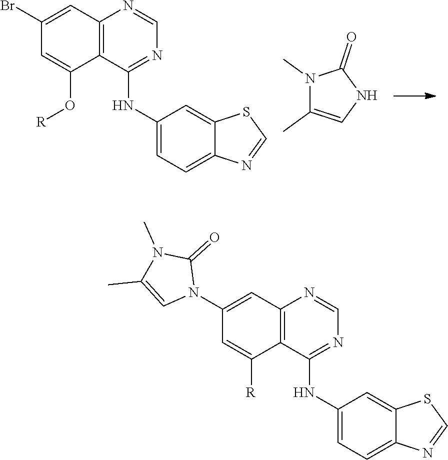

To a reaction vessel was added: The aryl halide (1 eq), the appropriate N-hetercycle (1.5 eq), BINAP (0.05 eq), Pd2dba₃, (0.03 eq), sodium t-Butoxide (3 eq) and DMA. Sparged reaction with nitrogen then heated to 95° C. for 6 hr. Stood at RT overnight. The reaction was concentrated, dissolved in DMSO and purified by prep HPLC to afford the desired title compound.

### Method B5i: Buchwald XPhos and BINAP

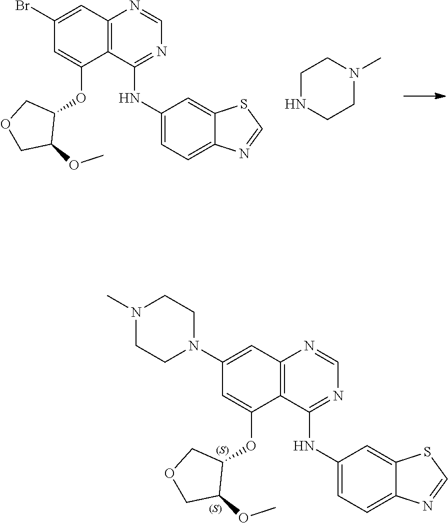

To a vial was added: N-(7-bromo-5-((3S-4S-methoxytetrahydrofuran-3-yl)oxy)quinazolin-4-yl)benzo[d]thiazol-6-amine (100 mg, 0.211 mmol), 1-methylpiperazine (42.3 mg, 0.423 mmol), rac-2,2′-Bis(diphenylphosphino)-1,1′-binaphthyl (6.58 mg, 10.56 μmol), Tris(dibenzylideneacetone)dipalladium(0) (9.67 mg, 10.56 μmol), XPhos Pd G3 (8.94 mg, 10.56 μmol), Sodium t-Butoxide (40.6 mg, 0.423 mmol) and DMA (2 ml). The reaction was sparged with nitrogen then heated the reaction to 100° C. for 16 hr. Filtered and purified by prep-HPLC to afford the desired product.

### Method B6: Borylation 6 Membered

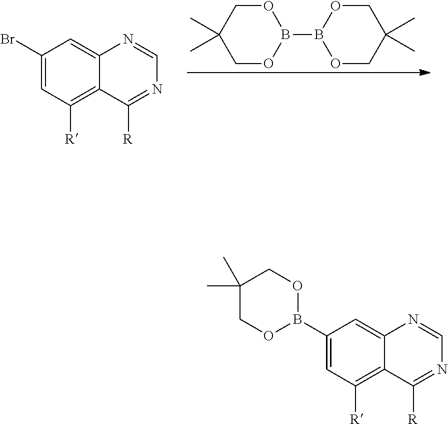

To a solution of the appropriate aryl halide (1.00 eq) in dioxane was added: 2-(5,5-dimethyl-1,3,2-dioxaborinan-2-yl)-5,5-dimethyl-1,3,2-dioxaborinane (1.50 eq), KOAc or KOtBu (2.00 eq) and Pd(dppf)Cl₂ (0.10 eq) under nitrogen. The mixture was stirred at 100° C. for 2-3 hours. The reaction mixture was cooled to room temperature and concentrated under vacuum. The product was purified by normal phase chromatography (DCM:MeOH=10:1) or by reverse phase chromatography (ACN in water; 0-30%) to afford the desired product.

### Method B7: Borylation Bpin,

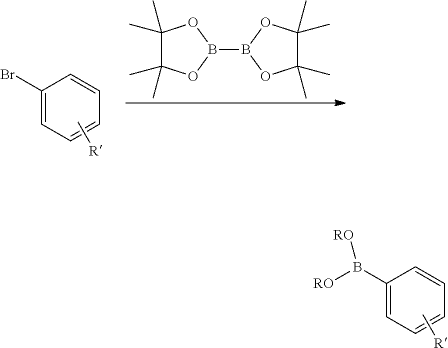

To the reaction vessel was added: KOAc (1.5-3 eq), Pd(dppf)Cl₂ (0.1 eq), an aryl halide (1 eq), 4,4,4′,4′,5,5,5′,5′-octamethyl-2,2′-bi(1,3,2-dioxaborolane) (1.2-2 eq) and DMSO at room temperature. The reaction mixture was heated to 60-100° C. for 2-4 h under N₂. The resulting solution was extracted with EA. The organic layer was dried with Na₂SO₄ and concentrated under vacuum. The crude product was purified by prep-TLC or silica column chromatography with DCM:MeOH=20:1 to afford the desired product.

Method C1: Reductive Amination with STAB

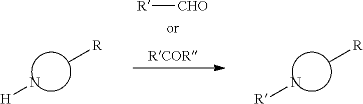

The alkyl amine (1 eq), aldehyde or ketone (1-5 eq) were added to DCM (50 mL) at rt. STAB (2 eq) was added and the reaction stirred for 1-2 h. The resulting solution was concentrated under vacuum then purified directly. Alternatively the reaction was diluted with EA or DCM and washed sequentially with water then saturated brine. The organic layer was dried over Na₂SO₄, filtered and evaporated. The crude product was purified by Prep-TLC (DCM:MeOH=15:1) or prep-HPLC to afford the desired product.

Method T4: Reductive Methylation with HCHO/NaCNBH₃

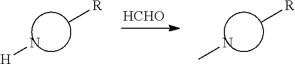

To a reaction vessel was added: amine (1.00 eq), methanol, HOAc (0 eq or 0.1-6.5 eq), formaldehyde (1.25-10 eq) and NaBH₃CN (1-3 eq). The resulting solution was stirred between 0° C. and rt for 1-10 hours as needed. The reaction was quenched by the addition of water or saturated NH₄Cl. The resulting solution was concentrated under vacuum then purified directly. Alternatively the reaction was diluted with EA or DCM and washed sequentially with water then saturated brine. The organic layer was dried over Na₂SO₄, filtered and evaporated. The crude product was purified by prep-HPLC to afford the desired product.

Method T4b: Reductive Amination with NaCNBH₃

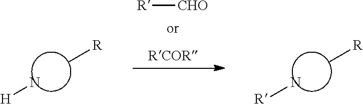

To the mixture of the appropriate amine (1 eq) in MeOH was added: an aldehyde or keytone (1-10 eq) and HOAc (a drop), the mixture was stirred for 30-180 minutes at 20° C.-rt, then NaBH₃CN (2-10 eq) was added. The mixture was stirred for 1 hr at 60° C., Alternately rt for 0.5-10 hours. The reaction was quenched by the addition of water or saturated NH₄Cl. The resulting solution was concentrated under vacuum then purified directly. Alternatively the reaction was diluted with EA or DCM and washed sequentially with water then saturated brine. The organic layer was dried over Na₂SO₄, filtered and evaporated. The crude product was purified by prep-HPLC to afford the desired product.

### Method T4C: Reductive Amination

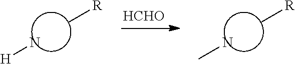

A solution of amine (1 eq) in MeOH was adjusted to pH=8 with DIPEA (1 eq) followed by adjusting the pH to 6 with AcOH (1-5 eq). HCHO (1 eq) was added to the reaction mixture, and stirred at 15-25° C. for 0.5-1 hr. NaBH₃CN (1.5-3 eq) was added to the reaction mixture and stirred at 25° C. for 1 hr. The mixture was concentrated in vacuum. The residue was purified by prep-HPLC to afford the desired product.

### Method C2: POCl₃

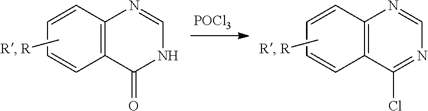

To a solution of 3,4-dihydroquinazolin-4-one (300 mg, 964 μmol) and DIEA (2-3 eq) or diethylaniline (5 eq), in ACN was added POCl₃ (3-5 eq) under N₂. The mixture was stirred at 80° C. for 1-3 hours. The reaction was:
a. Quenched by the addition of NaHCO₃ aq). The solids were filtered out and the resulting solution was extracted with DCM and the organic layers concentrated under vacuum.
b. Concentrated under vacuum and adjusted to PH=7 with NaHCO₃(aq). The crude material was purified by Prep-TLC (PE:EA=1:1) to afford the desired product.
c. Concentrated and taken to the next step without purification.

### Method D1: TBAF De Protection

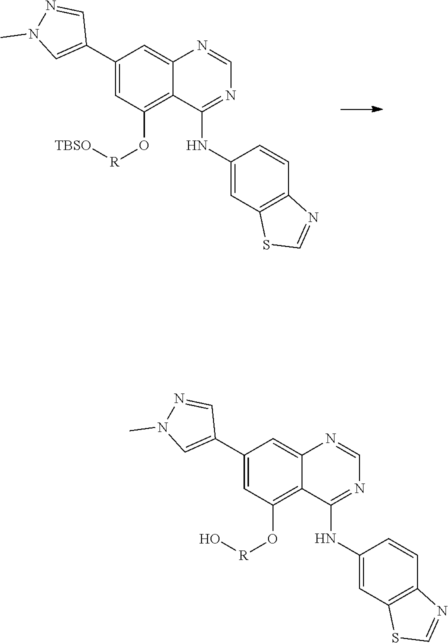

Into a vial was added: A TBS protected alcohol in CH₂Cl₂, was added tetra-n-butylammonium fluoride (excess) The resulting solution was stirred at RT for 2 h. The reaction was concentrated and the residue purified by prep-TLC with DCM/MeOH (5:1). The crude product was further purified by Prep-HPLC to afford the desired product.

### Method D2: Deprotection of Acid Labile Protecting Groups

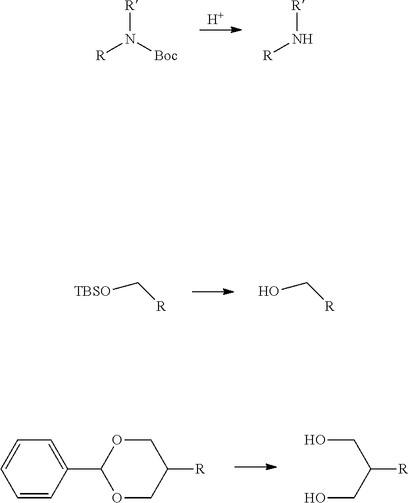

The protected compound (Typically a Boc group) was dissolved in an aprotic solvent (typically DCM or EA) then treated with excess HCl (anhydrous) or TFA. The reaction was stirred at rt for 1-16 hours.

The reaction was:
a. concentrated to afford the desired compound and used without further manipulation,
b. concentrated and purified to afford the desired compound.
c. neutralized with TEA or aqueous base, extracted with EA or DCM, washed with brine, dried over MgSO₄ and concentrated. The residue was further purified if necessary to afford the desired compound.
d. concentrated, diluted with methanol, adjusted to pH=7 with TEA and concentrated. The residue was further purified if necessary to afford the desired compound.

### Method T1:

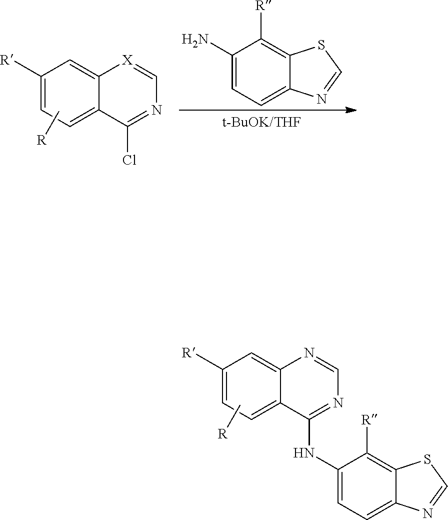

To the amino-heterocycle (0.8-2 eq) in THF was added t-BuOK (1 M, 1.2-3 eq) at 0° C. The reaction was stirred for 15 minutes. The 4-chloroquinazoline (1 eq) was added and the mixture was stirred at 25-80° C. for 2-12 hrs. The reaction mixture was:
a. concentrated and the residue was triturated with 1:1 THF:H₂O or 2:1 DMF:H₂O and collected the solid desired product.
b. concentrated and the residue was purified by silica gel chromatography afford the desired product.
c. diluted with ethyl acetate or DCM, washed with water and saturated brine. The organic layer was dried over Na₂SO₄, filtered, concentrated and the crude product was purified by silica gel chromatography to afford the desired compound.

### Method T1b:

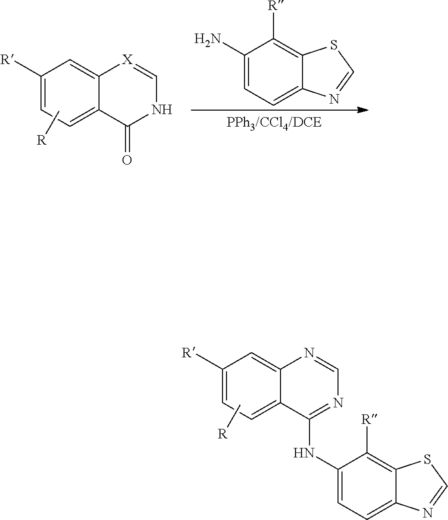

To a solution of the quinazolin-4(3H)-one (1 eq) in DCE was added PPh₃ (3 eq) and CCl₄ (6 eq), the reaction was heated to 75-80° C. for 1-2 hrs. under N₂. The amino-benzothiazole (1 eq) was added and the reaction mixture was stirred at 25° C. for 1-2 hr.
a. If solid product precipitated, and was of sufficient purity it was collected by filtration to afford the desired compound.
b. If not, then the reaction was concentrated and the residue was purified by prep-HPLC to afford the desired product.

### Method Tic: 1

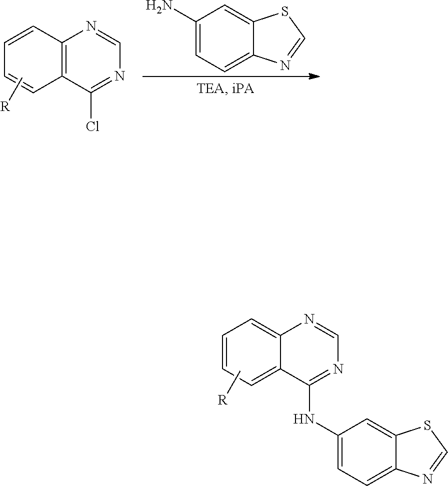

TEA (3 eq) was added to the quinazolin-4(3H)-one (1 eq) and amino-benzothiazole (1.6 eq) in IPA (0.4 N) at rt. The reaction mixture was heated to 80° C. for 2 h. Typically the reaction mixture was filtered, the solid collected and dried to afford the desired product.

### Method T1d

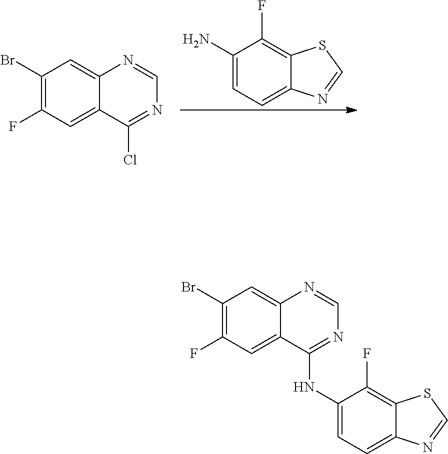 [SMILES: Fc1cc2c(Cl)ncnc2cc1Br ; Nc1ccc2ncsc2c1F ; Fc1cc2c(Nc3ccc4ncsc4c3F)ncnc2cc1Br]

NaH (2 eq) was added to 7-fluoro-L3-benzothiazol-6-amine (1 eq) in DMF and stirred for 10 min at It 7-bromo-4-chloro-6-fluoroquinazoline (1 eq) was added and the reaction was stirred for 10 h. The reaction was poured into ice-water, and the desired product was collected by filtration to afford the title compound.

### Method T6: Acylation:

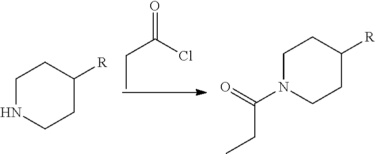

To a solution of amine (1 eq) in DCM was added DIPEA (1-3 eq) then acyl halide (1-3 eq) at 0° C. The mixture was stirred at 25° C. for 1 hr. The reaction mixture was filtered, concentrated and purified by prep-HPLC.

### Method T7: Urea Formation

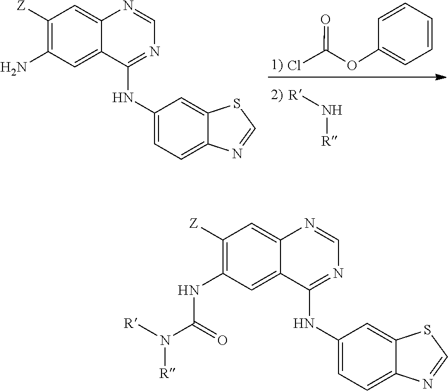

Step 1: To the mixture of arylamine (1 eq) in DMF was added pyridine (2 eq) and phenyl carbonochloridate (1-1.5 eq) at rt. The mixture was stirred at 70° C. for 2 hrs. The alkylamine (1-1.5 eq) was added and the mixture was stirred at 70° C. for 2 hrs. The mixture was filtered and the filtrate was purified by prep-HPLC to afford the title compound.

### Method T7B: F-Phenyl Amine Reagent.

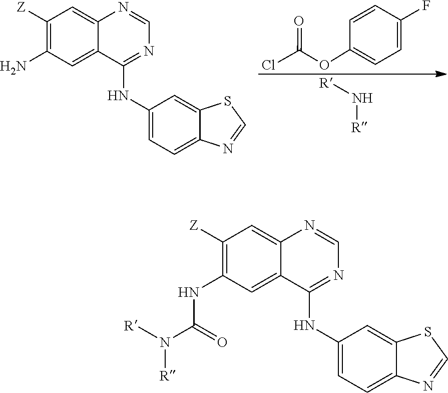

A solution of arylamine, (1 eq) in 2:1 DCM:pyridine was added DMAP (2-3 equiv), followed by 4-fluorophenyl carbonochloridate (10 eq) dropwise at 10° C.-20° C. The reaction was stirred at r.t. for 30 min. A solution of the amine (12-20 eq) in DMF was added to the above mixture. The reaction was stirred at r.t. for 15 min-3 h. The mixture was concentrated. The residue was purified by prep-TLC (DCM/MeOH=15:1) to afford the desired product.

### Method T7c: Urea Formation:

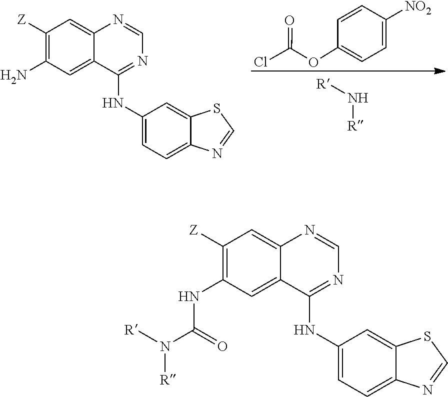

The arylamine in DCM was stirred with (1 (4-nitrophenyl) carbonochloridate (8 eq), pyridine (8 eq) and DMAP (2 eq) at rt for 30 minutes. The reaction mixture was added to a solution of alkylamine in DMF. Stirred at rt for 15-30 minutes. The reaction mixture was diluted with DCM, washed with H₂O (×3). The organic layer was dried over Na₂SO₄, filtered and evaporated to afford the title compound.

### Method T8: Copper Coupling:

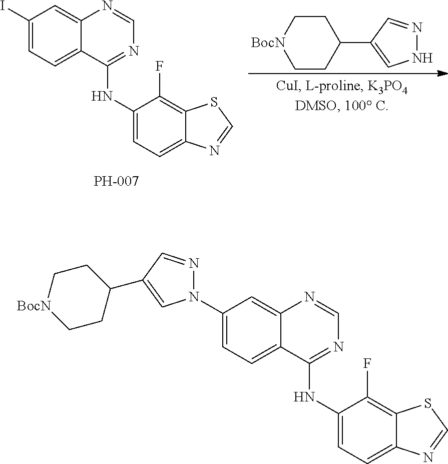 [SMILES: Fc1c(Nc2ncnc3cc(I)ccc23)ccc2ncsc12 ; CC(C)(C)OC(=O)N1CCC(c2cn[nH]c2)CC1 ; CC(C)(C)OC(=O)N1CCC(c2cnn(-c3ccc4c(Nc5ccc6ncsc6c5F)ncnc4c3)c2)CC1]

In a 8 ml sealed tube, N-(7-fluoro-1,3-benzothiazol-6-yl)-7-iodoquinazolin-4-amine (150 mg, 35 5 umol) tert-butyl 4-(1H-pyrazol-4-yl)piperidine-1-carboxylate (89.2 mg, 355 umol), CuI (33.6 mg, 177 umol), K₃PO₄ (224 mg, 1.06 mmol), L-Proline (8.16 mg, 71.0 umol) in DMSO (3 ml) were added under nitrogen and warmed to 120° C. for 12 h. The reaction mixture was diluted with water, and extracted with EA and saturated brine. The organic layer was dried over Na₂SO₄, filtered, evaporated, and purified by column chromatography (DCM:MeOH=10:1) to afford tert-butyl 4-(1-{4-[(7-fluoro-1,3-benzothiazol-6-yl)amino]quinazolin-7-yl}-1H-pyrazol-4-yl)piperidine-1-carboxylate (70 mg, 36.2%) as a yellow solid.

### Method E1: Ester Hydrolysis

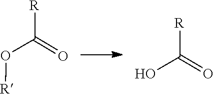

To a solution of ester (1 eq) in THF and H₂O was added LiOH (3-10 eq) in portions at rt. The mixture was stirred for 2 hr at rt. The reaction mixture was concentrated and acidified by HCl (1.00 M) until pH=6, the mixture was filtered and the filter cake was dried under reduced pressure to give the desired product. In cases where the product did not precipitate or was not of desired purity, the mixture was diluted with water and extracted with EA. The organic layer was combined and was dried over Na₂SO₄. The solution was filtered, concentrated. The residue was purified by silica gel CC, eluting with DCM/MeOH (5:1) to afford the desired products.

### Method E2: HATU Amide Coupling

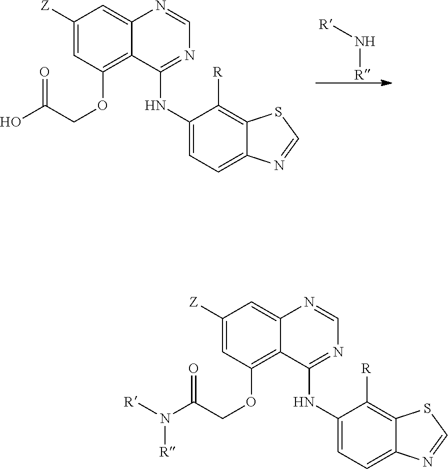

The carboxylic acid (1 eq), amine (1.5-2 eq), HATU (1.5 eq) and DIEA (1.5-5 eq) in DMF at rt. The reaction was stirred for 2-3 hr at rt. The mixture was extracted with EA and the combined organic layers were washed with water then brine. The organic layers were combined and was dried over Na₂SO₄. The solution was filtered, concentrated. The residue was purified by prep HPLC to isolate the desired product.

### Method E4: CMPI Amide Coupling

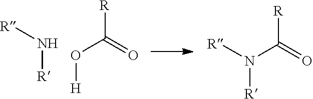

Into a reaction vessel was added: the amine (50 mg, 154 umol), the carboxylic acid (1.5 eq), CMPI (1.5 eq) and DIEA (1.5 eq) in DMF at rt. The mixture was stirred 2-16 hours at 60° C. The mixture was concentrated dissolved in EA and washed with water. The organic layer was dried over Na₂SO₄ and concentrated. The product was purified by prep HPLC.

### Method ES: NaBH4 Reduction of Ester

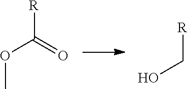

NaBH₄ (2 eq) was added to the ester (1 eq) in THF/MeOH (10 mL/1 mL) at 0-25° C. and stirred for 10-30 min. The resulting mixture was stirred at rt-80° C. for 1-15 h. The mixture was quenched with ice water, diluted with EA, washed with brine, the organic layer dried over Na₂SO₄ and concentrated under vacuum. The residue was purified by normal phase chromatography or prep HPLC as was needed.

### Preparation of Examples

The following Tables show how to prepare the exemplified compounds.

TABLE 3

| Example | | |
|---|---|---|
| # | Structure | Starting Material |
| 1 | 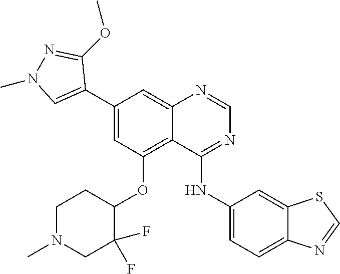 [SMILES: COc1nn(C)cc1-c1cc(OC2CCN(C)CC2(F)F)c2c(Nc3ccc4ncsc4c3)ncnc2c1] | 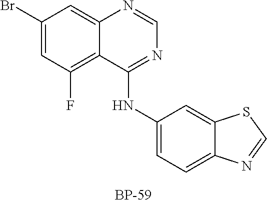 [SMILES: Fc1cc(Br)cc2ncnc(Nc3ccc4ncsc4c3)c12 ; "BP-59"] |

| | | | Methods |
|---|---|---|---|
| Example | | | (In order |
| # | Reactant 1 | Reactant 2 | used) |
| 1 | 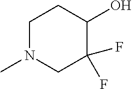 | 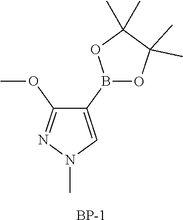 | 25 A1, B1 |

To illustrate, Example 1 was prepared by the following process:
Step 1: Method A1 (Methods column), Intermediate BP-59 (Starting Material column) and the alcohol (Reactant 1 column).
Step 2: The product of step 1 was coupled to Intermediate BP-1 (Reactant 2 column) using the conditions found in method B1 (Methods column) to afford the desired product, Example 1.
The order that the reactions were used is reflected by the order listed in the Methods column.
Key changes to a method or any notes are included in the Methods column as necessary.
Some reactions (such as acid deprotection D2) do not require any additional reactants in the table.
Conditions for all chiral separations are listed in a separate table.

An alternate depiction for the synthesis of Example 1:

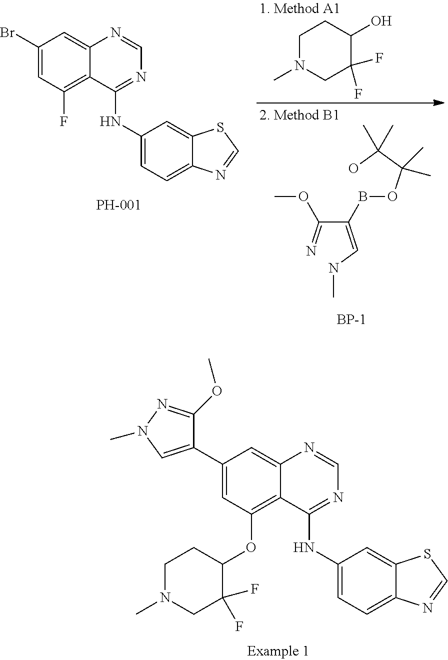

TABLE 4

| | | | | | | Methods |
|---|---|---|---|---|---|---|
| | | | | | | (In Order |
| # | Structure | SM | Reactant 1 | Reactant 2 | Reactant 3 | of use) |
| 325 | 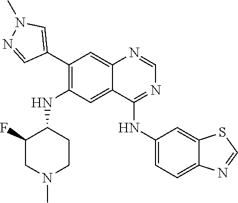 [SMILES: CN1CC[C@@H](Nc2cc3c(Nc4ccc5ncsc5c4)ncnc3cc2-c2cnn(C)c2)[C@H](F)C1] | 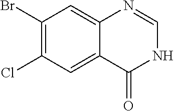 [SMILES: O=c1[nH]cnc2cc(Br)c(Cl)cc12] | 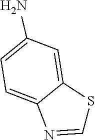 Method T1 | 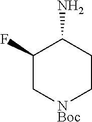 [SMILES: CC(C)(C)OC(=O)N1CC[C@@H](N)[C@H](F)C1] trans- racemic Method B5g | 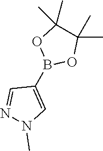 Method B1 | C2, T1, B5g, B1, D2, T4, Chiral Separation |
| | or | | | | | |
| 326 | 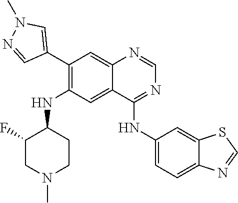 [SMILES: CN1CC[C@H](Nc2cc3c(Nc4ccc5ncsc5c4)ncnc3cc2-c2cnn(C)c2)[C@@H](F)C1] | | | | | |

An alternate depiction for the synthesis of Examples 325/326:

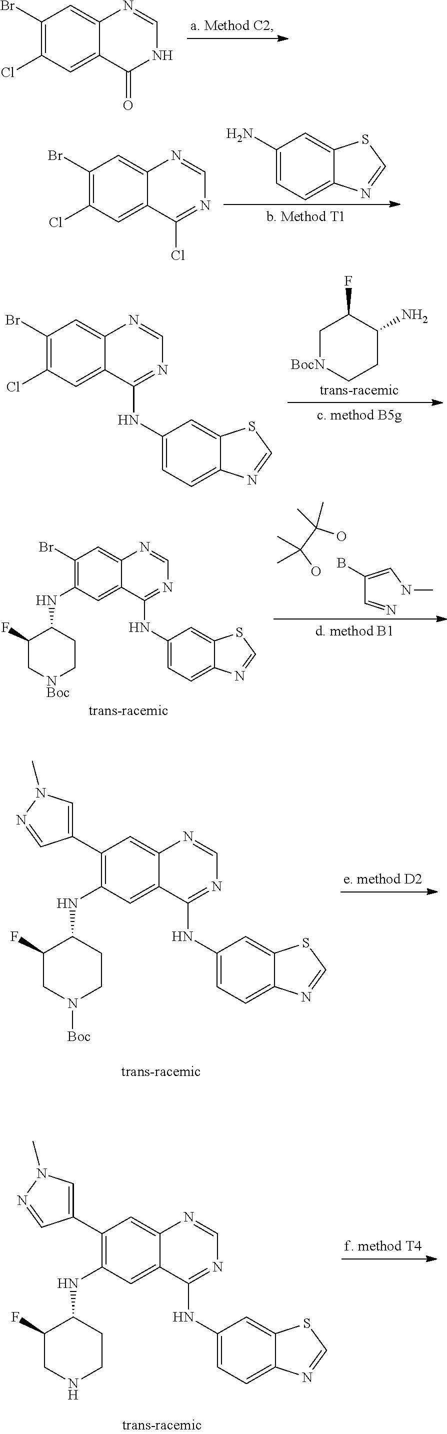

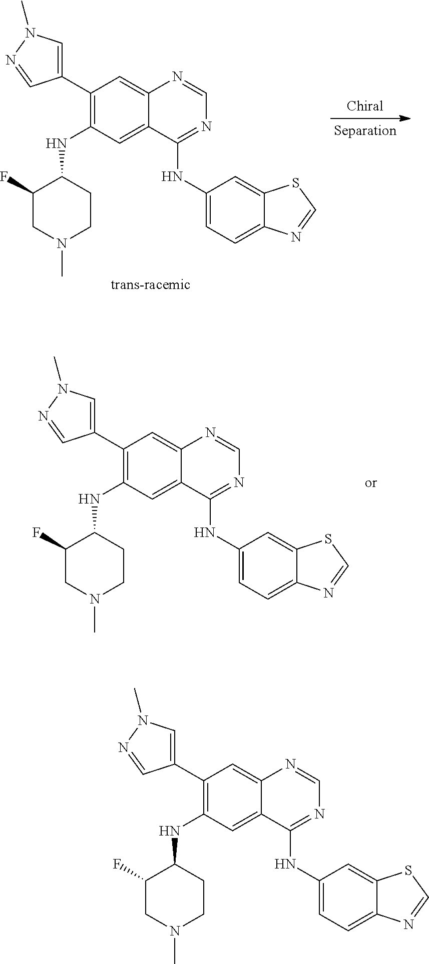 [SMILES: CN1CC[C@@H](Nc2cc3c(Nc4ccc5ncsc5c4)ncnc3cc2-c2cnn(C)c2)[C@H](F)C1 ; CN1CC[C@@H](Nc2cc3c(Nc4ccc5ncsc5c4)ncnc3cc2-c2cnn(C)c2)[C@H](F)C1 ; CN1CC[C@H](Nc2cc3c(Nc4ccc5ncsc5c4)ncnc3cc2-c2cnn(C)c2)[C@@H](F)C1]

TABLE 5

| Ex- | | | | |
|---|---|---|---|---|
| ample | | | | |
| # | Structure | Starting Material | | Reactant 1 |
| 1 | 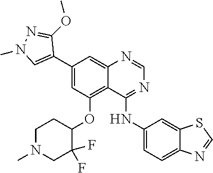 [SMILES: COc1nn(C)cc1-c1cc(OC2CCN(C)CC2(F)F)c2c(Nc3ccc4ncsc4c3)ncnc2c1] | 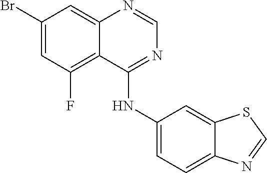 [SMILES: Fc1cc(Br)cc2ncnc(Nc3ccc4ncsc4c3)c12] | | 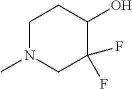 |
| | | BP-59 | | |
| 2 | 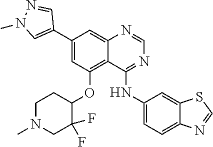 [SMILES: CN1CCC(Oc2cc(-c3cnn(C)c3)cc3ncnc(Nc4ccc5ncsc5c4)c23)C(F)(F)C1] | 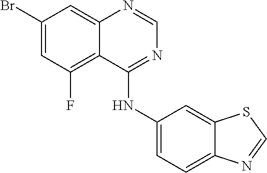 [SMILES: Fc1cc(Br)cc2ncnc(Nc3ccc4ncsc4c3)c12] | | 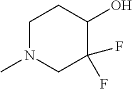 |
| | | BP-59 | | |
| 3 | 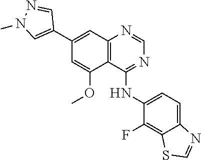 [SMILES: COc1cc(-c2cnn(C)c2)cc2ncnc(Nc3ccc4ncsc4c3F)c12] | 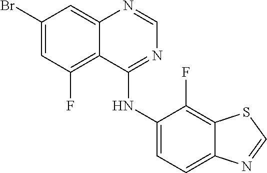 [SMILES: Fc1c(Nc2ncnc3cc(Br)cc(F)c23)ccc2ncsc12] | | Methanol |
| | | BP-60 | | |
| 4 | 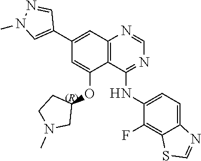 | 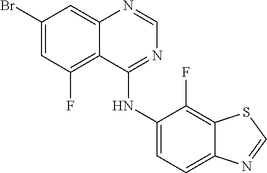 [SMILES: Fc1c(Nc2ncnc3cc(Br)cc(F)c23)ccc2ncsc12] | | 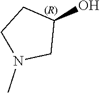 |
| | | BP-60 | | |
| 5 | 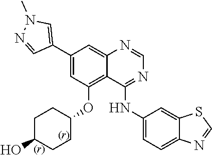 [SMILES: Cn1cc(-c2cc(O[C@H]3CC[C@H](O)CC3)c3c(Nc4ccc5ncsc5c4)ncnc3c2)cn1] | 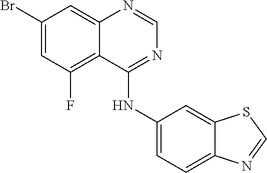 [SMILES: Fc1cc(Br)cc2ncnc(Nc3ccc4ncsc4c3)c12] | | 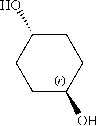 |
| | | BP-59 | | |
| 6 7 | 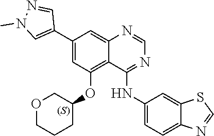 or 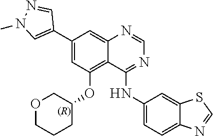 | 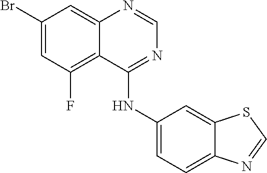 [SMILES: Fc1cc(Br)cc2ncnc(Nc3ccc4ncsc4c3)c12] BP-59 | | 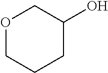 |
| 8 | 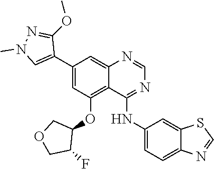 [SMILES: COc1nn(C)cc1-c1cc(O[C@@H]2COC[C@H]2F)c2c(Nc3ccc4ncsc4c3)ncnc2c1] and | 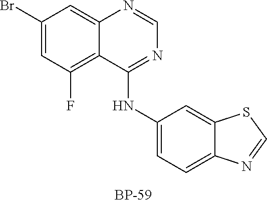 [SMILES: Fc1cc(Br)cc2ncnc(Nc3ccc4ncsc4c3)c12 ; "BP-59"] | | 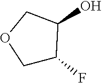 trans- racemic |
| | 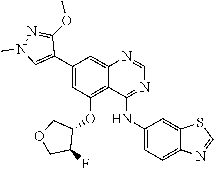 [SMILES: COc1nn(C)cc1-c1cc(O[C@H]2COC[C@@H]2F)c2c(Nc3ccc4ncsc4c3)ncnc2c1] | | | |
| 9 | 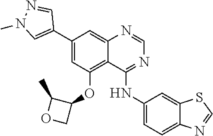 [SMILES: C[C@@H]1OC[C@@H]1Oc1cc(-c2cnn(C)c2)cc2ncnc(Nc3ccc4ncsc4c3)c12] cis-racemic | 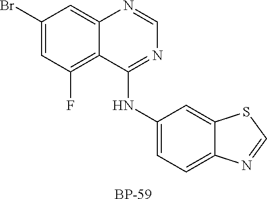 [SMILES: Fc1cc(Br)cc2ncnc(Nc3ccc4ncsc4c3)c12 ; "BP-59"] | | 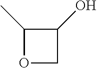 |
| 10 | 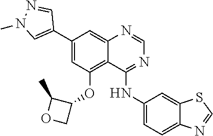 [SMILES: C[C@@H]1OC[C@H]1Oc1cc(-c2cnn(C)c2)cc2ncnc(Nc3ccc4ncsc4c3)c12] | | | |
| | trans-racemic | | | |
| 11 | 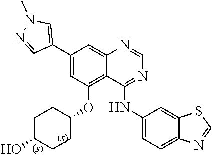 [SMILES: Cn1cc(-c2cc(O[C@H]3CC[C@@H](O)CC3)c3c(Nc4ccc5ncsc5c4)ncnc3c2)cn1] | 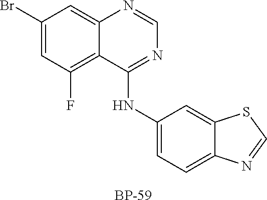 [SMILES: Fc1cc(Br)cc2ncnc(Nc3ccc4ncsc4c3)c12 ; "BP-59"] | | 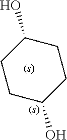 |
| 12 | 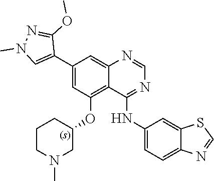 [SMILES: COc1nn(C)cc1-c1cc(O[C@H]2CCCN(C)C2)c2c(Nc3ccc4ncsc4c3)ncnc2c1] | 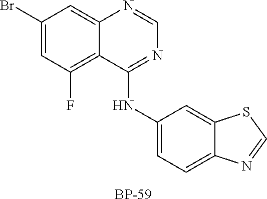 [SMILES: Fc1cc(Br)cc2ncnc(Nc3ccc4ncsc4c3)c12 ; "BP-59"] | | 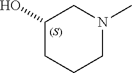 |
| 13 | 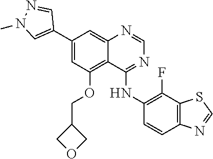 [SMILES: Cn1cc(-c2cc(OCC3COC3)c3c(Nc4ccc5ncsc5c4F)ncnc3c2)cn1] | 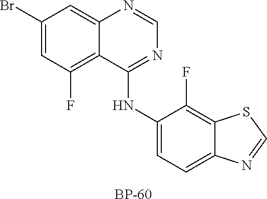 [SMILES: Fc1c(Nc2ncnc3cc(Br)cc(F)c23)ccc2ncsc12 ; "BP-60"] | | 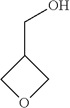 |
| 14 | 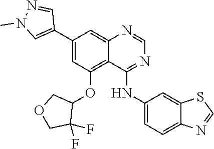 [SMILES: Cn1cc(-c2cc(OC3COCC3(F)F)c3c(Nc4ccc5ncsc5c4)ncnc3c2)cn1] | 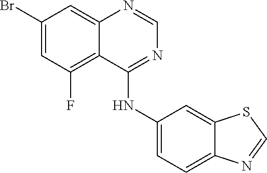 [SMILES: Fc1cc(Br)cc2ncnc(Nc3ccc4ncsc4c3)c12] | | 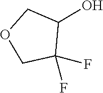 |
| | | BP-59 | | |
| 15 | 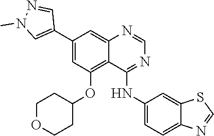 [SMILES: Cn1cc(-c2cc(OC3CCOCC3)c3c(Nc4ccc5ncsc5c4)ncnc3c2)cn1] | 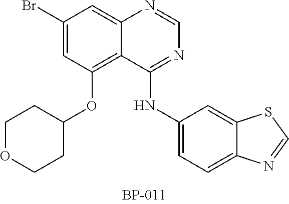 [SMILES: Brc1cc(OC2CCOCC2)c2c(Nc3ccc4ncsc4c3)ncnc2c1] | | 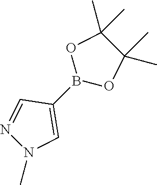 |
| 16 | 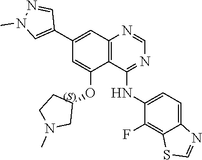 | 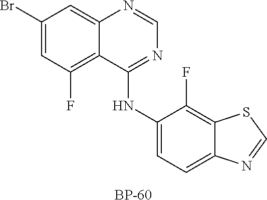 [SMILES: Fc1c(Nc2ncnc3cc(Br)cc(F)c23)ccc2ncsc12 ; "BP-60"] | | 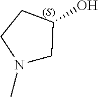 |
| 17 | 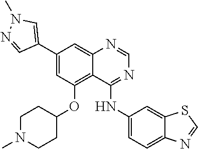 [SMILES: CN1CCC(Oc2cc(-c3cnn(C)c3)cc3ncnc(Nc4ccc5ncsc5c4)c23)CC1] | 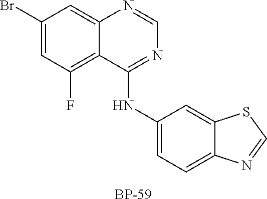 [SMILES: Fc1cc(Br)cc2ncnc(Nc3ccc4ncsc4c3)c12 ; "BP-59"] | | 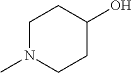 |
| 18 | 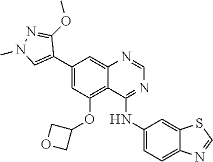 [SMILES: COc1nn(C)cc1-c1cc(OC2COC2)c2c(Nc3ccc4ncsc4c3)ncnc2c1] | 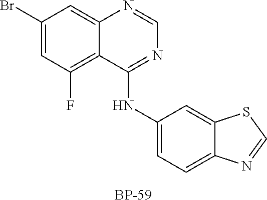 [SMILES: Fc1cc(Br)cc2ncnc(Nc3ccc4ncsc4c3)c12 ; "BP-59"] | | 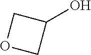 |
| 19 | 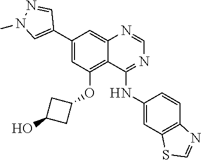 [SMILES: Cn1cc(-c2cc(O[C@H]3C[C@H](O)C3)c3c(Nc4ccc5ncsc5c4)ncnc3c2)cn1] | 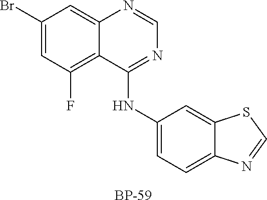 [SMILES: Fc1cc(Br)cc2ncnc(Nc3ccc4ncsc4c3)c12 ; "BP-59"] | | 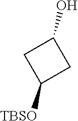 [SMILES: CC(C)(C)[Si](C)(C)O[C@H]1C[C@H](O)C1] |
| 20 | 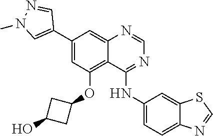 [SMILES: Cn1cc(-c2cc(O[C@H]3C[C@@H](O)C3)c3c(Nc4ccc5ncsc5c4)ncnc3c2)cn1] | 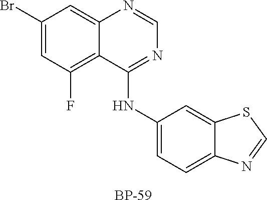 [SMILES: Fc1cc(Br)cc2ncnc(Nc3ccc4ncsc4c3)c12 ; "BP-59"] | | 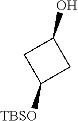 [SMILES: CC(C)(C)[Si](C)(C)O[C@H]1C[C@@H](O)C1] |
| 21 | 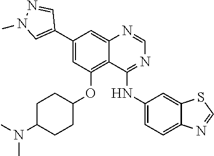 [SMILES: CN(C)C1CCC(Oc2cc(-c3cnn(C)c3)cc3ncnc(Nc4ccc5ncsc5c4)c23)CC1] | 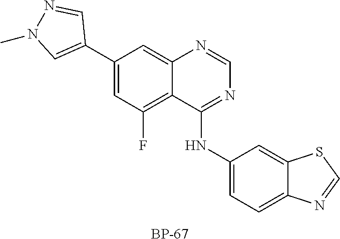 [SMILES: Cn1cc(-c2cc(F)c3c(Nc4ccc5ncsc5c4)ncnc3c2)cn1 ; "BP-67"] | | 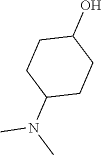 |
| 22 | 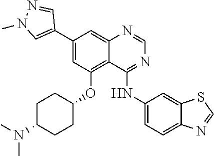 [SMILES: Cn1cc(-c2cc(O[C@H]3CC[C@@H](N(C)C)CC3)c3c(Nc4ccc5ncsc5c4)ncnc3c2)cn1] | 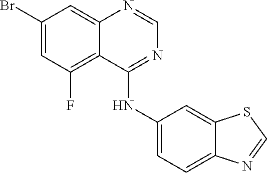 [SMILES: Fc1cc(Br)cc2ncnc(Nc3ccc4ncsc4c3)c12] | | 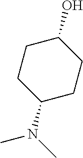 |
| | | BP-59 | | |
| 23 24 | 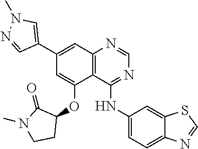 [SMILES: CN1CC[C@H](Oc2cc(-c3cnn(C)c3)cc3ncnc(Nc4ccc5ncsc5c4)c23)C1=O] Or 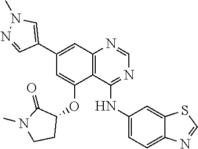 [SMILES: CN1CC[C@@H](Oc2cc(-c3cnn(C)c3)cc3ncnc(Nc4ccc5ncsc5c4)c23)C1=O] | 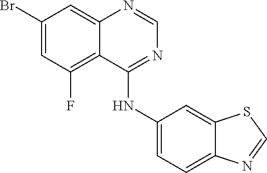 [SMILES: Fc1cc(Br)cc2ncnc(Nc3ccc4ncsc4c3)c12] BP-59 | | 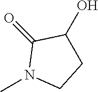 |
| 25 | 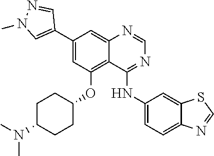 [SMILES: Cn1cc(-c2cc(O[C@H]3CC[C@@H](N(C)C)CC3)c3c(Nc4ccc5ncsc5c4)ncnc3c2)cn1] cis-racemic | 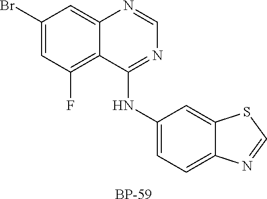 [SMILES: Fc1cc(Br)cc2ncnc(Nc3ccc4ncsc4c3)c12 ; "BP-59"] | | 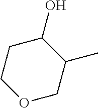 |
| 26 | 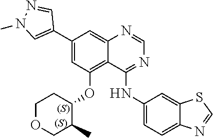 | | | |
| | trans-racemic | | | |
| 27 | 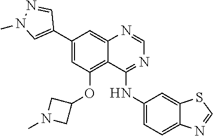 [SMILES: CN1CC(Oc2cc(-c3cnn(C)c3)cc3ncnc(Nc4ccc5ncsc5c4)c23)C1] | 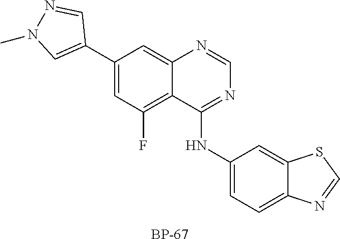 [SMILES: Cn1cc(-c2cc(F)c3c(Nc4ccc5ncsc5c4)ncnc3c2)cn1 ; "BP-67"] | | 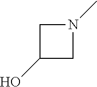 |
| 28 | 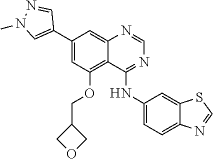 [SMILES: Cn1cc(-c2cc(OCC3COC3)c3c(Nc4ccc5ncsc5c4)ncnc3c2)cn1] | 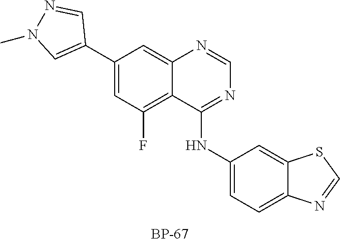 [SMILES: Cn1cc(-c2cc(F)c3c(Nc4ccc5ncsc5c4)ncnc3c2)cn1 ; "BP-67"] | | 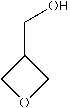 |
| 29 | 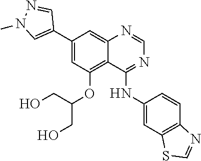 [SMILES: Cn1cc(-c2cc(OC(CO)CO)c3c(Nc4ccc5ncsc5c4)ncnc3c2)cn1] | 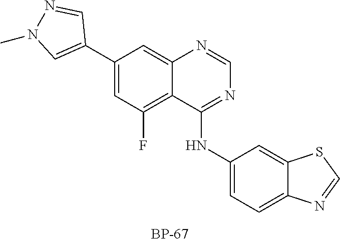 [SMILES: Cn1cc(-c2cc(F)c3c(Nc4ccc5ncsc5c4)ncnc3c2)cn1 ; "BP-67"] | | 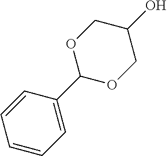 [SMILES: OC1COC(c2ccccc2)OC1] |
| 30 | 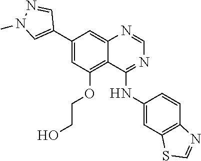 [SMILES: Cn1cc(-c2cc(OCCO)c3c(Nc4ccc5ncsc5c4)ncnc3c2)cn1] | 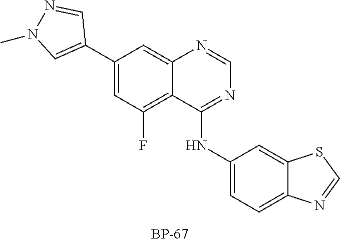 [SMILES: Cn1cc(-c2cc(F)c3c(Nc4ccc5ncsc5c4)ncnc3c2)cn1 ; "BP-67"] | | 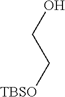 [SMILES: CC(C)(C)[Si](C)(C)OCCO] |
| 31 | 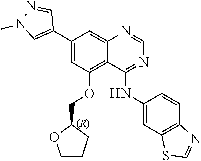 | 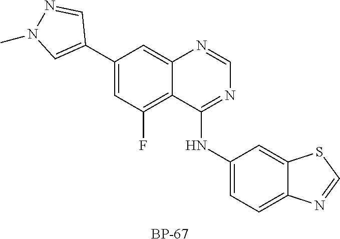 [SMILES: Cn1cc(-c2cc(F)c3c(Nc4ccc5ncsc5c4)ncnc3c2)cn1 ; "BP-67"] | | 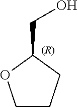 |
| 32 | 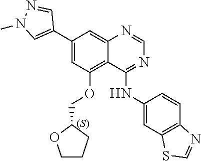 | 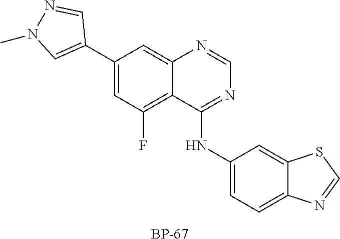 [SMILES: Cn1cc(-c2cc(F)c3c(Nc4ccc5ncsc5c4)ncnc3c2)cn1 ; "BP-67"] | | 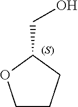 |
| 33 | 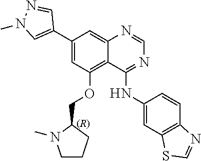 | 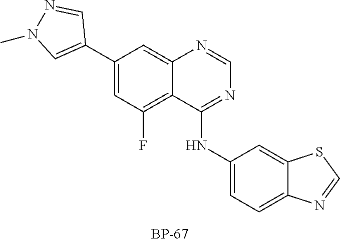 [SMILES: Cn1cc(-c2cc(F)c3c(Nc4ccc5ncsc5c4)ncnc3c2)cn1 ; "BP-67"] | | 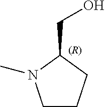 |
| 34 | 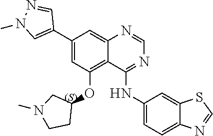 | 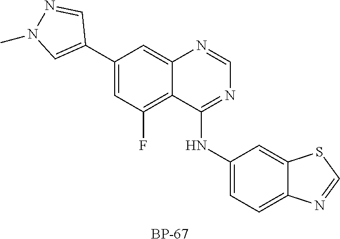 [SMILES: Cn1cc(-c2cc(F)c3c(Nc4ccc5ncsc5c4)ncnc3c2)cn1 ; "BP-67"] | | 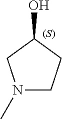 |
| 35 | 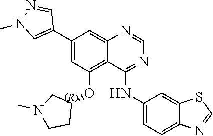 | 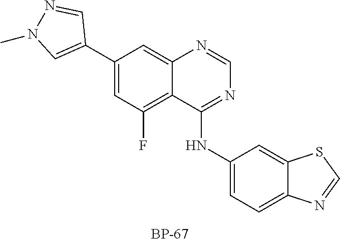 [SMILES: Cn1cc(-c2cc(F)c3c(Nc4ccc5ncsc5c4)ncnc3c2)cn1 ; "BP-67"] | | |
| 36 | 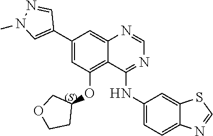 | 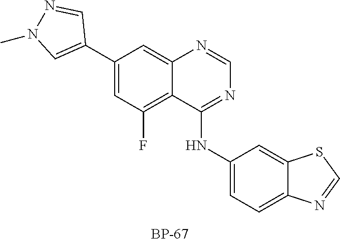 [SMILES: Cn1cc(-c2cc(F)c3c(Nc4ccc5ncsc5c4)ncnc3c2)cn1 ; "BP-67"] | | 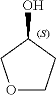 |
| 37 | 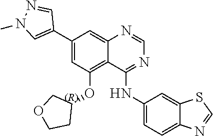 | 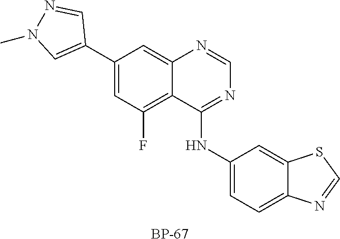 [SMILES: Cn1cc(-c2cc(F)c3c(Nc4ccc5ncsc5c4)ncnc3c2)cn1 ; "BP-67"] | | 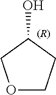 |
| 38 | 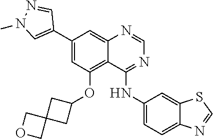 [SMILES: Cn1cc(-c2cc(OC3CC4(COC4)C3)c3c(Nc4ccc5ncsc5c4)ncnc3c2)cn1] | 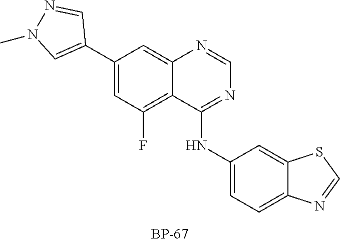 [SMILES: Cn1cc(-c2cc(F)c3c(Nc4ccc5ncsc5c4)ncnc3c2)cn1 ; "BP-67"] | | 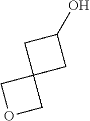 |
| 41 42 | 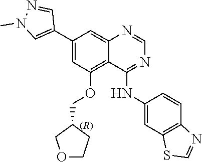 Or 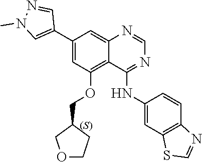 | 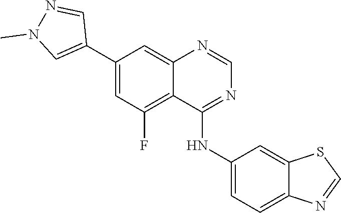 [SMILES: Cn1cc(-c2cc(F)c3c(Nc4ccc5ncsc5c4)ncnc3c2)cn1] BP-67 | | 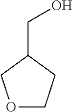 |
| 43 44 | 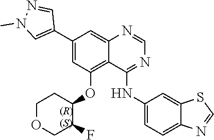 Or 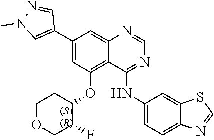 | 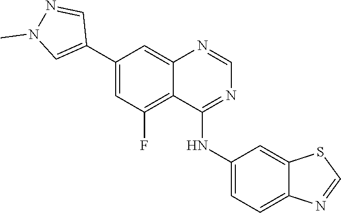 [SMILES: Cn1cc(-c2cc(F)c3c(Nc4ccc5ncsc5c4)ncnc3c2)cn1] BP-67 | | 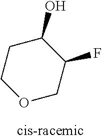 |
| 45 46 | 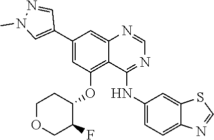 [SMILES: Cn1cc(-c2cc(O[C@H]3CCOC[C@@H]3F)c3c(Nc4ccc5ncsc5c4)ncnc3c2)cn1] Or 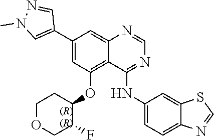 | 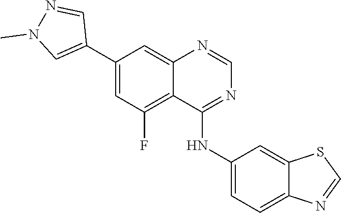 [SMILES: Cn1cc(-c2cc(F)c3c(Nc4ccc5ncsc5c4)ncnc3c2)cn1] BP-67 | | |
| 47 48 | 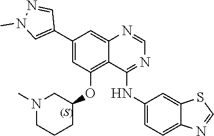 Or 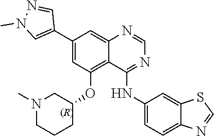 | 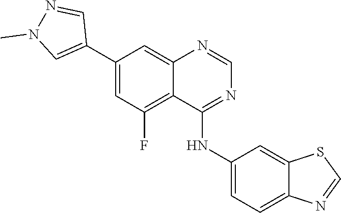 [SMILES: Cn1cc(-c2cc(F)c3c(Nc4ccc5ncsc5c4)ncnc3c2)cn1] BP-67 | | 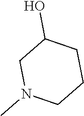 |
| 49 | 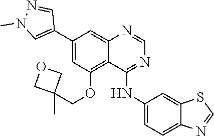 [SMILES: Cn1cc(-c2cc(OCC3(C)COC3)c3c(Nc4ccc5ncsc5c4)ncnc3c2)cn1] | 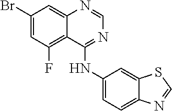 [SMILES: Fc1cc(Br)cc2ncnc(Nc3ccc4ncsc4c3)c12] BP-59 | | 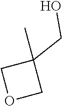 |
| 50 | 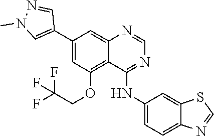 [SMILES: Cn1cc(-c2cc(OCC(F)(F)F)c3c(Nc4ccc5ncsc5c4)ncnc3c2)cn1] | 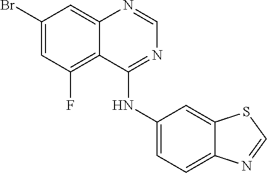 [SMILES: Fc1cc(Br)cc2ncnc(Nc3ccc4ncsc4c3)c12] BP-59 | | 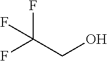 |
| 51 | 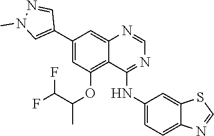 [SMILES: CC(Oc1cc(-c2cnn(C)c2)cc2ncnc(Nc3ccc4ncsc4c3)c12)C(F)F] | 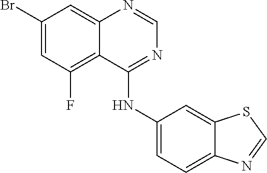 [SMILES: Fc1cc(Br)cc2ncnc(Nc3ccc4ncsc4c3)c12] BP-59 | | 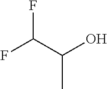 |
| 52 53 | 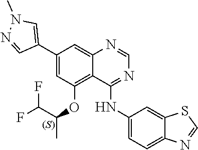 | Example 51 | | |
| | Or | | | |
| | 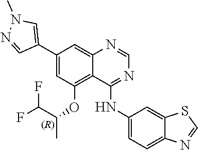 | | | |
| 54 | 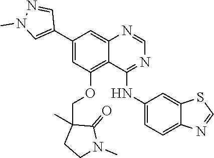 [SMILES: CN1CCC(C)(COc2cc(-c3cnn(C)c3)cc3ncnc(Nc4ccc5ncsc5c4)c23)C1=O] | 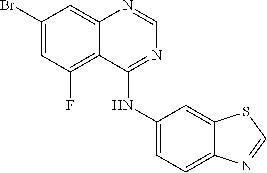 [SMILES: Fc1cc(Br)cc2ncnc(Nc3ccc4ncsc4c3)c12] BP-59 | | 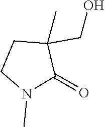 |
| 55 56 | 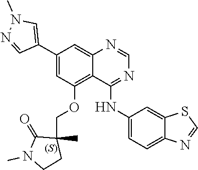 | Example 54 | | |
| | Or | | | |
| | 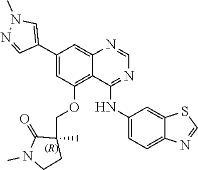 | | | |
| 57 | 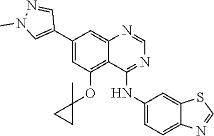 [SMILES: Cn1cc(-c2cc(OC3(C)CC3)c3c(Nc4ccc5ncsc5c4)ncnc3c2)cn1] | 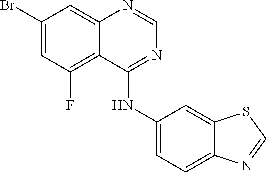 [SMILES: Fc1cc(Br)cc2ncnc(Nc3ccc4ncsc4c3)c12] | | 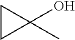 |
| | | BP-59 | | |
| 58 | 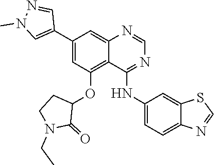 [SMILES: CCN1CCC(Oc2cc(-c3cnn(C)c3)cc3ncnc(Nc4ccc5ncsc5c4)c23)C1=O] | 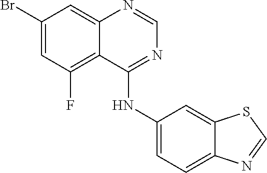 [SMILES: Fc1cc(Br)cc2ncnc(Nc3ccc4ncsc4c3)c12] | | 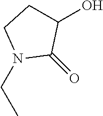 BP-5 |
| | | BP-59 | | |
| 59 60 | 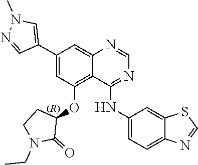 | Example 58 | | |
| | Or | | | |
| | 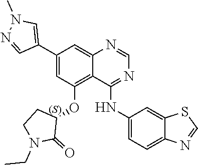 | | | |
| 61 | 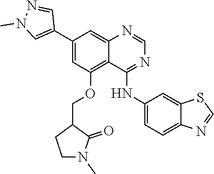 [SMILES: CN1CCC(COc2cc(-c3cnn(C)c3)cc3ncnc(Nc4ccc5ncsc5c4)c23)C1=O] | 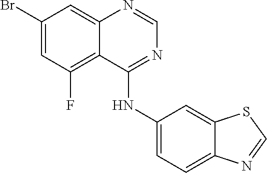 [SMILES: Fc1cc(Br)cc2ncnc(Nc3ccc4ncsc4c3)c12] | | 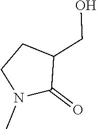 |
| | | BP-59 | | |
| 62 | 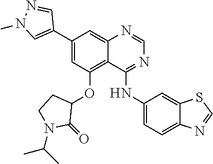 [SMILES: CC(C)N1CCC(Oc2cc(-c3cnn(C)c3)cc3ncnc(Nc4ccc5ncsc5c4)c23)C1=O] | 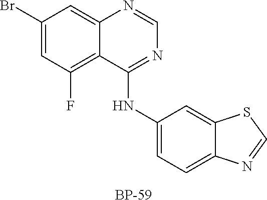 [SMILES: Fc1cc(Br)cc2ncnc(Nc3ccc4ncsc4c3)c12 ; "BP-59"] | | 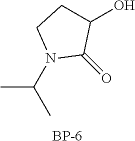 BP-6 |
| 63 64 | 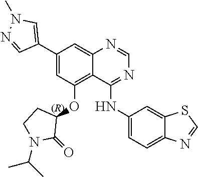 | Example 62 | | — |
| | Or | | | |
| | 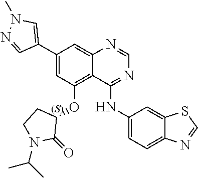 | | | |
| 65 | 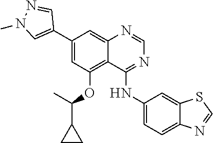 [SMILES: C[C@@H](Oc1cc(-c2cnn(C)c2)cc2ncnc(Nc3ccc4ncsc4c3)c12)C1CC1] | 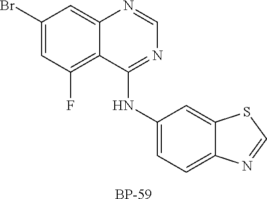 [SMILES: Fc1cc(Br)cc2ncnc(Nc3ccc4ncsc4c3)c12 ; "BP-59"] | | 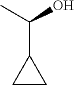 |
| 66 | 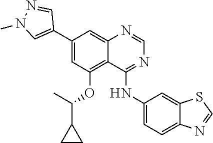 [SMILES: C[C@H](Oc1cc(-c2cnn(C)c2)cc2ncnc(Nc3ccc4ncsc4c3)c12)C1CC1] | 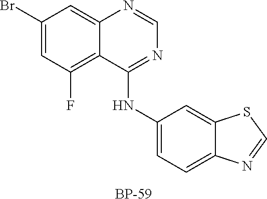 [SMILES: Fc1cc(Br)cc2ncnc(Nc3ccc4ncsc4c3)c12 ; "BP-59"] | | 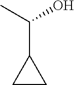 |
| 67 | 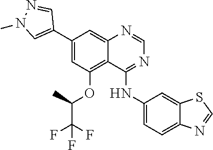 [SMILES: C[C@@H](Oc1cc(-c2cnn(C)c2)cc2ncnc(Nc3ccc4ncsc4c3)c12)C(F)(F)F] | 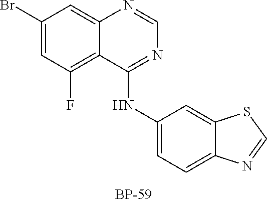 [SMILES: Fc1cc(Br)cc2ncnc(Nc3ccc4ncsc4c3)c12 ; "BP-59"] | | 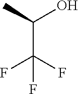 |
| 68 | 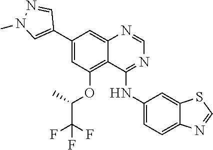 [SMILES: C[C@H](Oc1cc(-c2cnn(C)c2)cc2ncnc(Nc3ccc4ncsc4c3)c12)C(F)(F)F] | 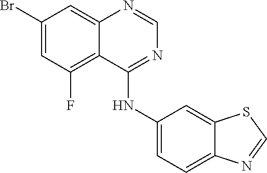 [SMILES: Fc1cc(Br)cc2ncnc(Nc3ccc4ncsc4c3)c12] | | 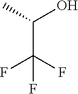 |
| | | BP-59 | | |
| 69 | 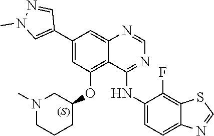 | 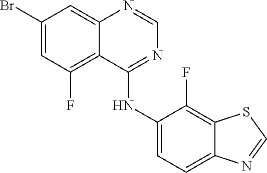 [SMILES: Fc1c(Nc2ncnc3cc(Br)cc(F)c23)ccc2ncsc12] | | 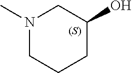 |
| | | BP-60 | | |
| 70 71 | 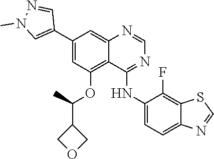 [SMILES: C[C@@H](Oc1cc(-c2cnn(C)c2)cc2ncnc(Nc3ccc4ncsc4c3F)c12)C1COC1] | 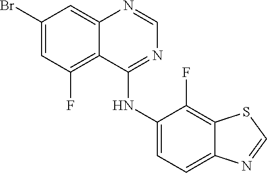 [SMILES: Fc1c(Nc2ncnc3cc(Br)cc(F)c23)ccc2ncsc12] | | 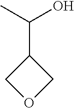 |
| | Or | | | |
| | | BP-60 | | |
| | 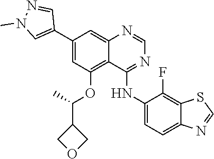 [SMILES: C[C@H](Oc1cc(-c2cnn(C)c2)cc2ncnc(Nc3ccc4ncsc4c3F)c12)C1COC1] | | | |
| 72 73 | 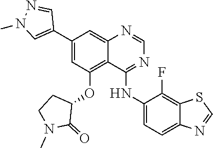 [SMILES: CN1CC[C@H](Oc2cc(-c3cnn(C)c3)cc3ncnc(Nc4ccc5ncsc5c4F)c23)C1=O] Or 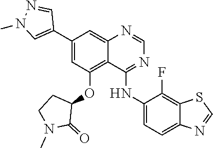 [SMILES: CN1CC[C@@H](Oc2cc(-c3cnn(C)c3)cc3ncnc(Nc4ccc5ncsc5c4F)c23)C1=O] | 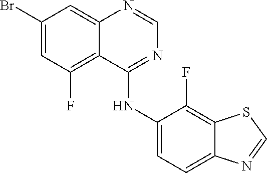 [SMILES: Fc1c(Nc2ncnc3cc(Br)cc(F)c23)ccc2ncsc12] BP-60 | | 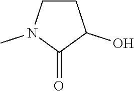 |
| 74 75 | 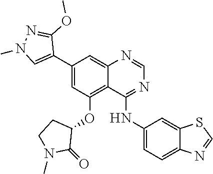 [SMILES: COc1nn(C)cc1-c1cc(O[C@H]2CCN(C)C2=O)c2c(Nc3ccc4ncsc4c3)ncnc2c1] Or | 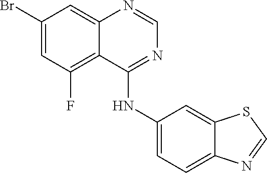 [SMILES: Fc1cc(Br)cc2ncnc(Nc3ccc4ncsc4c3)c12] BP-59 | | 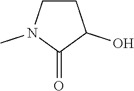 |
| | 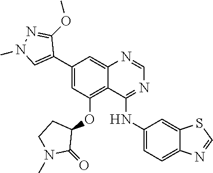 [SMILES: COc1nn(C)cc1-c1cc(O[C@@H]2CCN(C)C2=O)c2c(Nc3ccc4ncsc4c3)ncnc2c1] | | | |
| 76 | 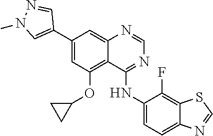 [SMILES: Cn1cc(-c2cc(OC3CC3)c3c(Nc4ccc5ncsc5c4F)ncnc3c2)cn1] | 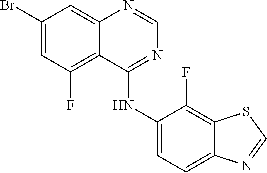 [SMILES: Fc1c(Nc2ncnc3cc(Br)cc(F)c23)ccc2ncsc12] | | 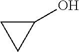 |
| | | BP-60 | | |
| 77 | 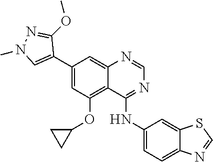 [SMILES: COc1nn(C)cc1-c1cc(OC2CC2)c2c(Nc3ccc4ncsc4c3)ncnc2c1] | 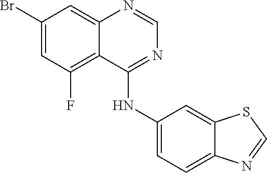 [SMILES: Fc1cc(Br)cc2ncnc(Nc3ccc4ncsc4c3)c12] | | 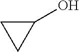 |
| | | BP-59 | | |
| 78 79 | 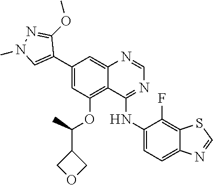 [SMILES: COc1nn(C)cc1-c1cc(O[C@H](C)C2COC2)c2c(Nc3ccc4ncsc4c3F)ncnc2c1] Or 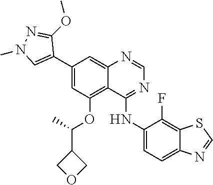 [SMILES: COc1nn(C)cc1-c1cc(O[C@@H](C)C2COC2)c2c(Nc3ccc4ncsc4c3F)ncnc2c1] | 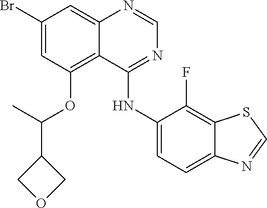 [SMILES: CC(Oc1cc(Br)cc2ncnc(Nc3ccc4ncsc4c3F)c12)C1COC1] BP-9 | | 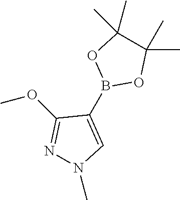 BP-1 |
| 80 81 | 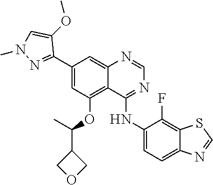 [SMILES: COc1cn(C)nc1-c1cc(O[C@H](C)C2COC2)c2c(Nc3ccc4ncsc4c3F)ncnc2c1] Or 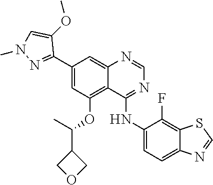 [SMILES: COc1cn(C)nc1-c1cc(O[C@@H](C)C2COC2)c2c(Nc3ccc4ncsc4c3F)ncnc2c1] | 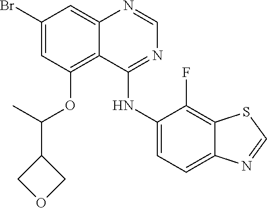 [SMILES: CC(Oc1cc(Br)cc2ncnc(Nc3ccc4ncsc4c3F)c12)C1COC1] BP-9 | | 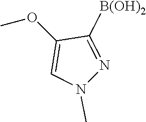 BP-4 |
| 82 83 | 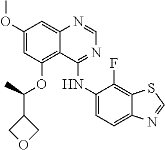 [SMILES: COc1cc(O[C@H](C)C2COC2)c2c(Nc3ccc4ncsc4c3F)ncnc2c1] Or 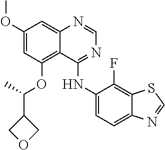 [SMILES: COc1cc(O[C@@H](C)C2COC2)c2c(Nc3ccc4ncsc4c3F)ncnc2c1] | 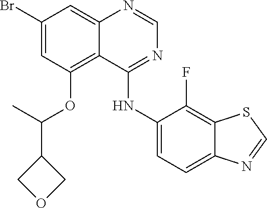 [SMILES: CC(Oc1cc(Br)cc2ncnc(Nc3ccc4ncsc4c3F)c12)C1COC1] BP-9 | | |
| 84 85 | 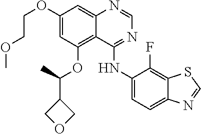 [SMILES: COCCOc1cc(O[C@H](C)C2COC2)c2c(Nc3ccc4ncsc4c3F)ncnc2c1] Or 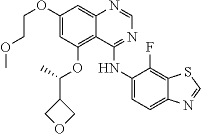 [SMILES: COCCOc1cc(O[C@@H](C)C2COC2)c2c(Nc3ccc4ncsc4c3F)ncnc2c1] | 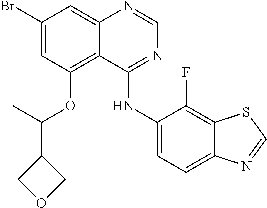 [SMILES: CC(Oc1cc(Br)cc2ncnc(Nc3ccc4ncsc4c3F)c12)C1COC1] BP-9 | | 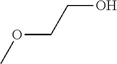 |
| 86 87 | 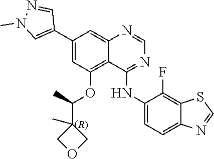 Or | 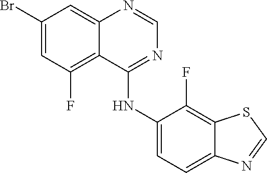 [SMILES: Fc1c(Nc2ncnc3cc(Br)cc(F)c23)ccc2ncsc12] BP-60 | | |
| | 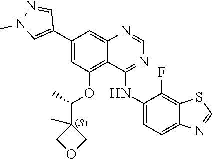 | | | |
| 88 89 | 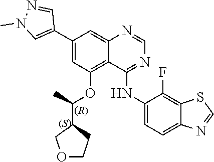 Or | 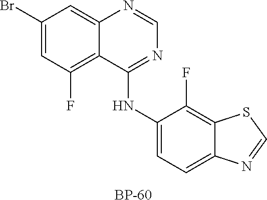 [SMILES: Fc1c(Nc2ncnc3cc(Br)cc(F)c23)ccc2ncsc12 ; "BP-60"] | | |
| | 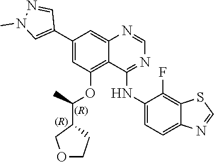 | | | |
| 90 | 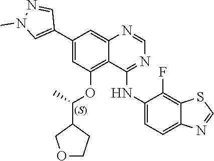 | 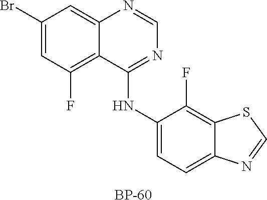 [SMILES: Fc1c(Nc2ncnc3cc(Br)cc(F)c23)ccc2ncsc12 ; "BP-60"] | | 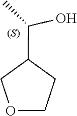 |
| 91 | 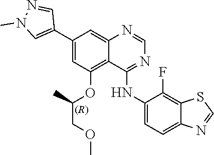 | 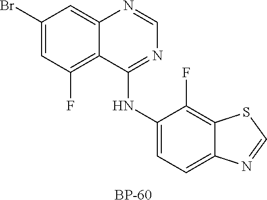 [SMILES: Fc1c(Nc2ncnc3cc(Br)cc(F)c23)ccc2ncsc12 ; "BP-60"] | | 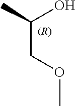 |
| 92 93 | 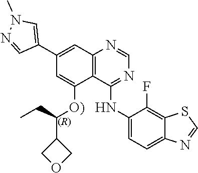 Or | 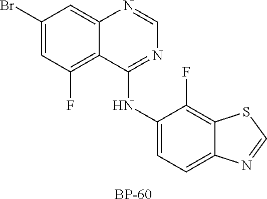 [SMILES: Fc1c(Nc2ncnc3cc(Br)cc(F)c23)ccc2ncsc12 ; "BP-60"] | | 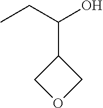 |
| | 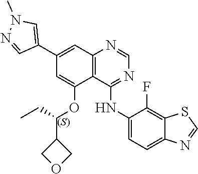 | | | |
| 94 95 | 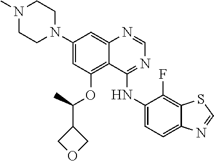 [SMILES: C[C@@H](Oc1cc(N2CCN(C)CC2)cc2ncnc(Nc3ccc4ncsc4c3F)c12)C1COC1] Or 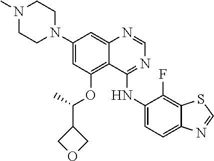 [SMILES: C[C@H](Oc1cc(N2CCN(C)CC2)cc2ncnc(Nc3ccc4ncsc4c3F)c12)C1COC1] | 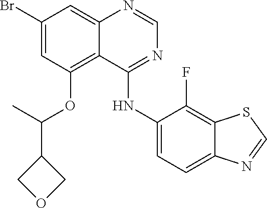 [SMILES: CC(Oc1cc(Br)cc2ncnc(Nc3ccc4ncsc4c3F)c12)C1COC1] BP-9 | | 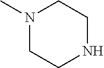 |
| 96 97 | 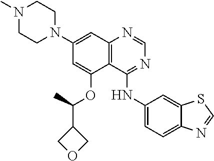 [SMILES: C[C@@H](Oc1cc(N2CCN(C)CC2)cc2ncnc(Nc3ccc4ncsc4c3)c12)C1COC1] Or 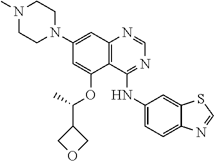 [SMILES: C[C@H](Oc1cc(N2CCN(C)CC2)cc2ncnc(Nc3ccc4ncsc4c3)c12)C1COC1] | 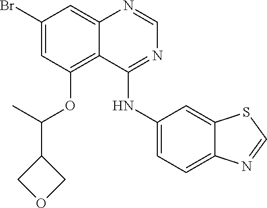 [SMILES: CC(Oc1cc(Br)cc2ncnc(Nc3ccc4ncsc4c3)c12)C1COC1] BP-8 | | 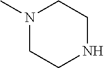 |
| 98 99 | 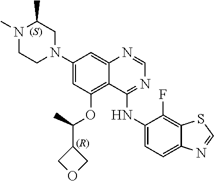 Or 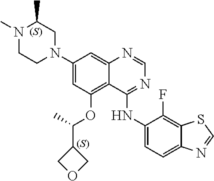 | 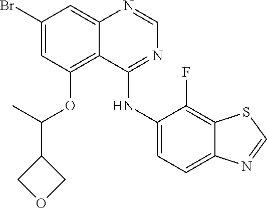 [SMILES: CC(Oc1cc(Br)cc2ncnc(Nc3ccc4ncsc4c3F)c12)C1COC1] BP-9 | | 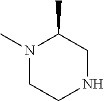 |
| 100 101 | 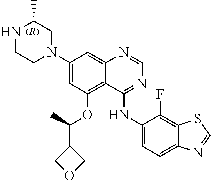 Or 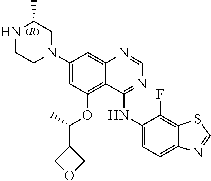 | 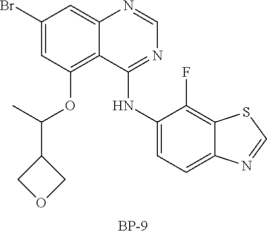 [SMILES: CC(Oc1cc(Br)cc2ncnc(Nc3ccc4ncsc4c3F)c12)C1COC1 ; "BP-9"] | | 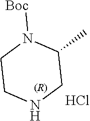 |
| 102 103 | 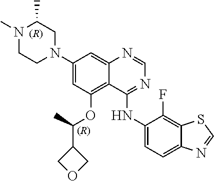 Or | 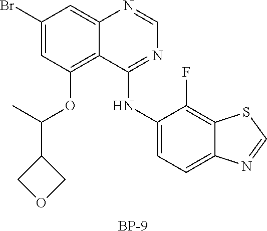 [SMILES: CC(Oc1cc(Br)cc2ncnc(Nc3ccc4ncsc4c3F)c12)C1COC1 ; "BP-9"] | | 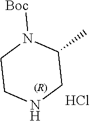 |
| | 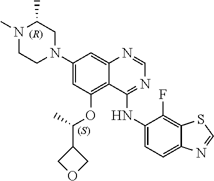 | | | |
| 104 105 | 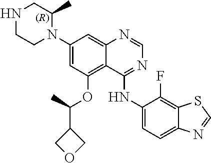 Or 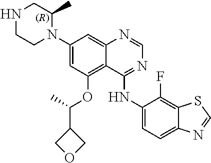 | - 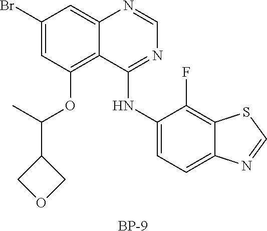 [SMILES: CC(Oc1cc(Br)cc2ncnc(Nc3ccc4ncsc4c3F)c12)C1COC1 ; "BP-9"] | | 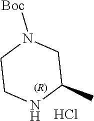 |
| 106 107 | 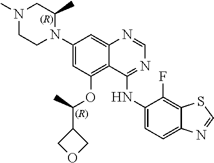 Or 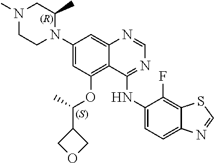 | 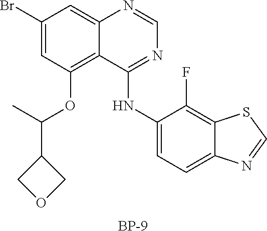 [SMILES: CC(Oc1cc(Br)cc2ncnc(Nc3ccc4ncsc4c3F)c12)C1COC1 ; "BP-9"] | | 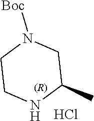 |
| 108 109 | 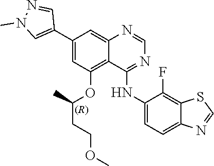 Or | 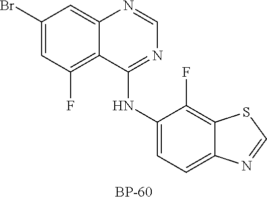 [SMILES: Fc1c(Nc2ncnc3cc(Br)cc(F)c23)ccc2ncsc12 ; "BP-60"] | | 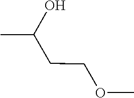 |
| | 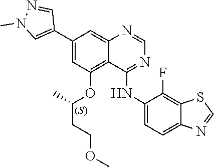 | | | |
| 110 111 | 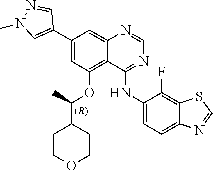 or | 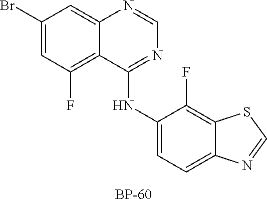 [SMILES: Fc1c(Nc2ncnc3cc(Br)cc(F)c23)ccc2ncsc12 ; "BP-60"] | | |
| | 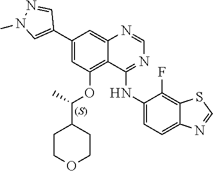 | | | |
| 112 | 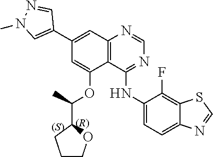 | 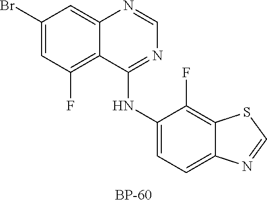 [SMILES: Fc1c(Nc2ncnc3cc(Br)cc(F)c23)ccc2ncsc12 ; "BP-60"] | | 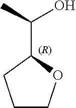 |
| 113 | 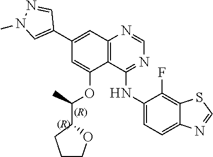 | 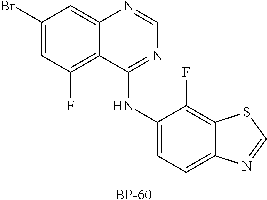 [SMILES: Fc1c(Nc2ncnc3cc(Br)cc(F)c23)ccc2ncsc12 ; "BP-60"] | | 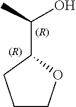 |
| 114 | 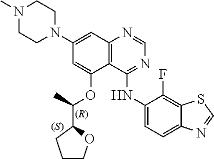 | 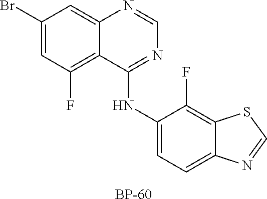 [SMILES: Fc1c(Nc2ncnc3cc(Br)cc(F)c23)ccc2ncsc12 ; "BP-60"] | | 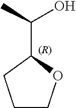 |
| 115 | 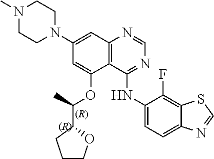 | 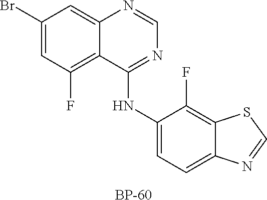 [SMILES: Fc1c(Nc2ncnc3cc(Br)cc(F)c23)ccc2ncsc12 ; "BP-60"] | | 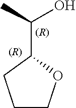 |
| 116 117 | 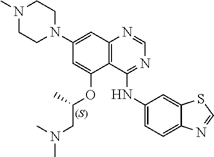 Or | 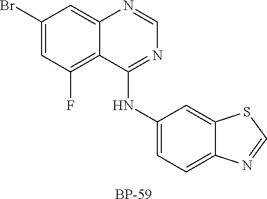 [SMILES: Fc1cc(Br)cc2ncnc(Nc3ccc4ncsc4c3)c12 ; "BP-59"] | | 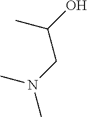 |
| | 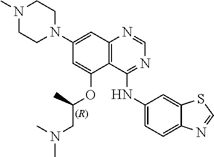 | | | |
| 118 119 | 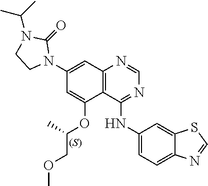 . | 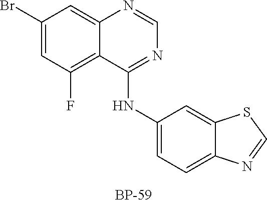 [SMILES: Fc1cc(Br)cc2ncnc(Nc3ccc4ncsc4c3)c12 ; "BP-59"] | | |
| | Or | | | |
| | 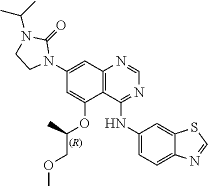 | | | |
| 120 121 | 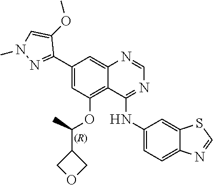 Or | 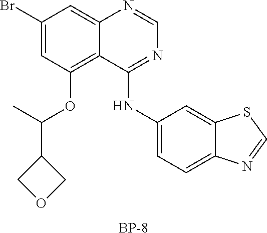 [SMILES: CC(Oc1cc(Br)cc2ncnc(Nc3ccc4ncsc4c3)c12)C1COC1 ; "BP-8"] | | 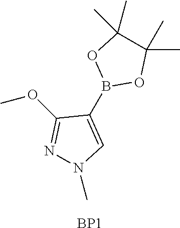 |
| | 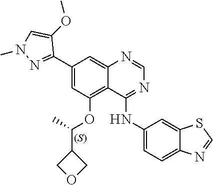 | | | |
| 122 123 | 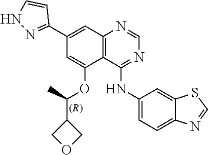 Or 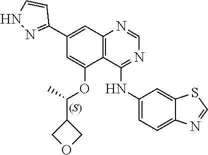 | 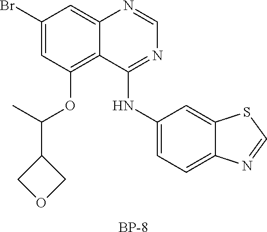 [SMILES: CC(Oc1cc(Br)cc2ncnc(Nc3ccc4ncsc4c3)c12)C1COC1 ; "BP-8"] | | 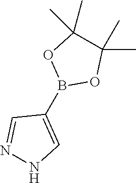 |
| 124 | 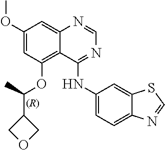 | 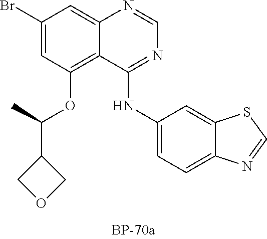 [SMILES: C[C@@H](Oc1cc(Br)cc2ncnc(Nc3ccc4ncsc4c3)c12)C1COC1] | | Methanol |
| 125 | 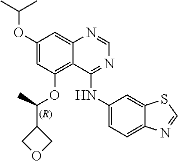 | 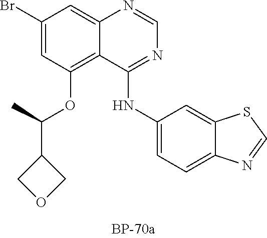 [SMILES: C[C@@H](Oc1cc(Br)cc2ncnc(Nc3ccc4ncsc4c3)c12)C1COC1] | | IPA |
| 126 | 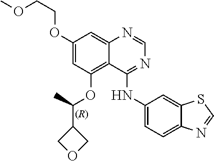 | 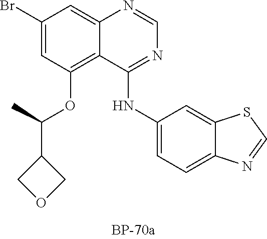 [SMILES: C[C@@H](Oc1cc(Br)cc2ncnc(Nc3ccc4ncsc4c3)c12)C1COC1] | | 2-methoxyethanol |
| 127 128 | 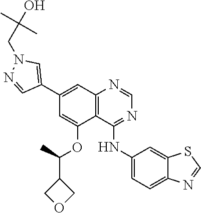 [SMILES: C[C@@H](Oc1cc(-c2cnn(CC(C)(C)O)c2)cc2ncnc(Nc3ccc4ncsc4c3)c12)C1COC1] | 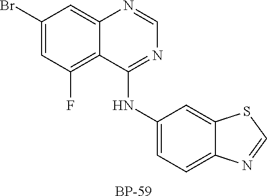 [SMILES: Fc1cc(Br)cc2ncnc(Nc3ccc4ncsc4c3)c12 ; "BP-59"] | | 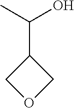 |
| | Or | | | |
| | 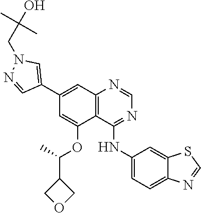 [SMILES: C[C@H](Oc1cc(-c2cnn(CC(C)(C)O)c2)cc2ncnc(Nc3ccc4ncsc4c3)c12)C1COC1] | | | |
| 129 | 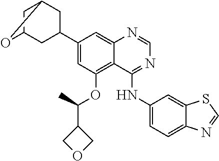 [SMILES: C[C@@H](Oc1cc(C2CC3CC(C2)O3)cc2ncnc(Nc3ccc4ncsc4c3)c12)C1COC1] | 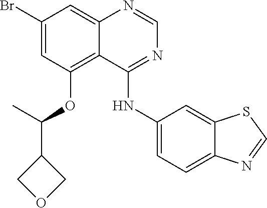 [SMILES: C[C@@H](Oc1cc(Br)cc2ncnc(Nc3ccc4ncsc4c3)c12)C1COC1] | | 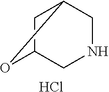 |
| | | BP-70a | | |
| 130 | 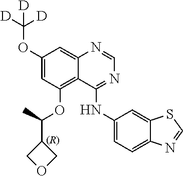 | 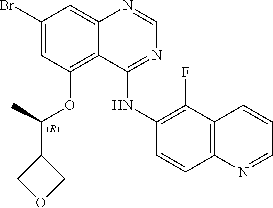 | | 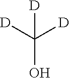 |
| | | BP-70a | | |
| 131 | 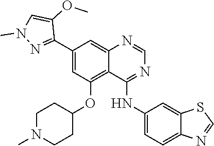 [SMILES: COc1cn(C)nc1-c1cc(OC2CCN(C)CC2)c2c(Nc3ccc4ncsc4c3)ncnc2c1] | 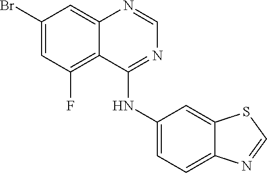 [SMILES: Fc1cc(Br)cc2ncnc(Nc3ccc4ncsc4c3)c12] | | 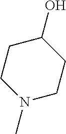 |
| | | BP-59 | | |
| 132 | 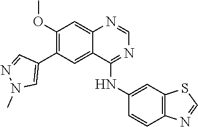 [SMILES: COc1cc2ncnc(Nc3ccc4ncsc4c3)c2cc1-c1cnn(C)c1] | 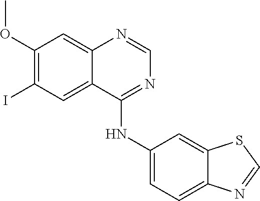 [SMILES: COc1cc2ncnc(Nc3ccc4ncsc4c3)c2cc1I] | | 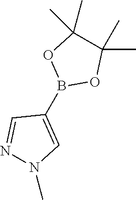 |
| | | BP-44 | | |
| 133 | 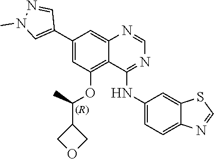 | 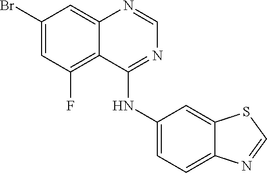 [SMILES: Fc1cc(Br)cc2ncnc(Nc3ccc4ncsc4c3)c12] | | 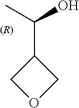 |
| | | BP-59 | | |
| 134 | 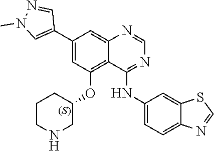 | 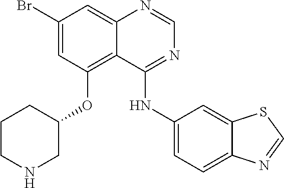 [SMILES: Brc1cc(O[C@H]2CCCNC2)c2c(Nc3ccc4ncsc4c3)ncnc2c1] | | 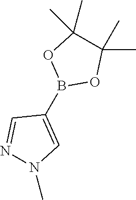 |
| | | BP-10 | | |
| 135 | 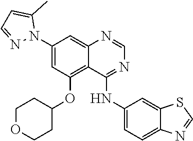 [SMILES: Cc1ccnn1-c1cc(OC2CCOCC2)c2c(Nc3ccc4ncsc4c3)ncnc2c1] | 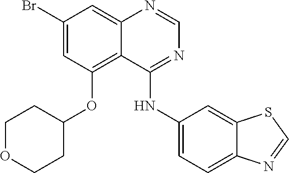 [SMILES: Brc1cc(OC2CCOCC2)c2c(Nc3ccc4ncsc4c3)ncnc2c1] | | 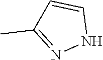 |
| 136 | 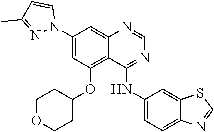 [SMILES: Cc1ccn(-c2cc(OC3CCOCC3)c3c(Nc4ccc5ncsc5c4)ncnc3c2)n1] | BP-011 | | |
| 139 | 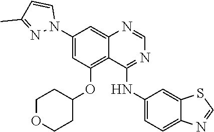 [SMILES: Cc1ccn(-c2cc(OC3CCOCC3)c3c(Nc4ccc5ncsc5c4)ncnc3c2)n1] | 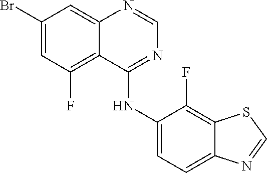 [SMILES: Fc1c(Nc2ncnc3cc(Br)cc(F)c23)ccc2ncsc12] | | 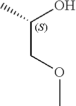 |
| | | BP-60 | | |
| 140 | 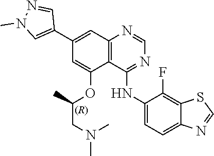 | 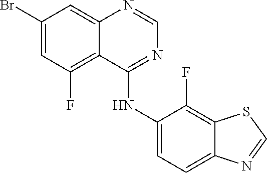 [SMILES: Fc1c(Nc2ncnc3cc(Br)cc(F)c23)ccc2ncsc12] | | 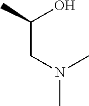 |
| | | BP-60 | | |
| 141 | 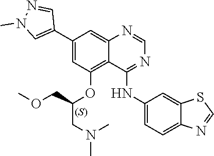 | 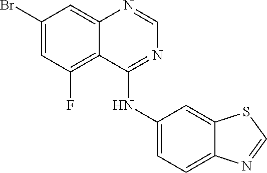 [SMILES: Fc1cc(Br)cc2ncnc(Nc3ccc4ncsc4c3)c12] | | 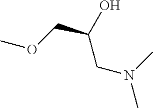 |
| | | BP-59 | | |
| 142 143 | 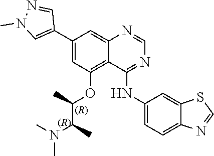 Or | 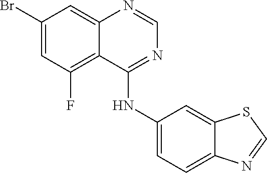 [SMILES: Fc1cc(Br)cc2ncnc(Nc3ccc4ncsc4c3)c12] BP-59 | | 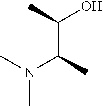 BP-13 racemic |
| | 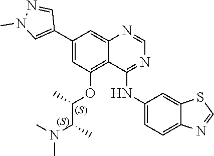 | | | |
| 144 145 | 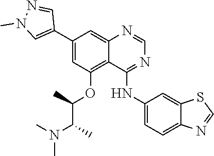 [SMILES: C[C@@H](Oc1cc(-c2cnn(C)c2)cc2ncnc(Nc3ccc4ncsc4c3)c12)[C@H](C)N(C)C] | 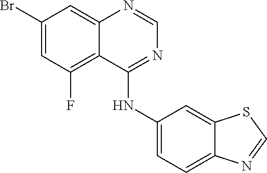 [SMILES: Fc1cc(Br)cc2ncnc(Nc3ccc4ncsc4c3)c12] | | 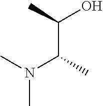 BP-14 racemic |
| | Or | | | |
| | | BP-59 | | |
| | 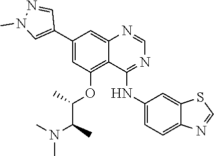 [SMILES: C[C@H](Oc1cc(-c2cnn(C)c2)cc2ncnc(Nc3ccc4ncsc4c3)c12)[C@@H](C)N(C)C] | | | |
| 146 | 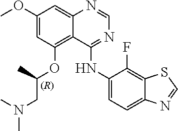 | 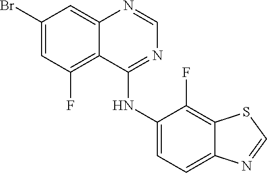 [SMILES: Fc1c(Nc2ncnc3cc(Br)cc(F)c23)ccc2ncsc12] BP-60 | | 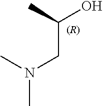 |
| 147 148 | 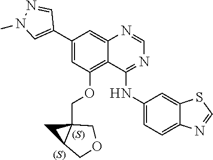 Or | 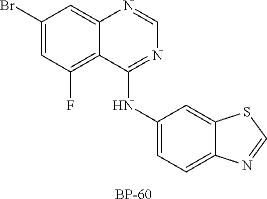 [SMILES: Fc1cc(Br)cc2ncnc(Nc3ccc4ncsc4c3)c12 ; "BP-60"] | | 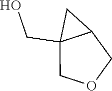 |
| | 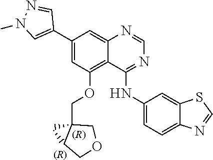 | | | |
| 149 150 | 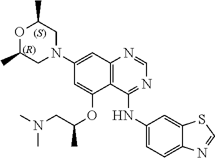 Or | 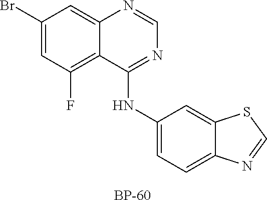 [SMILES: Fc1cc(Br)cc2ncnc(Nc3ccc4ncsc4c3)c12 ; "BP-60"] | | 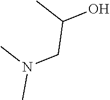 |
| | 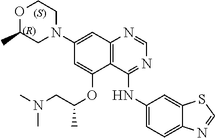 | | | |
| 151 152 | 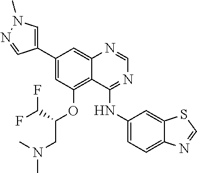 [SMILES: CN(C)C[C@@H](Oc1cc(-c2cnn(C)c2)cc2ncnc(Nc3ccc4ncsc4c3)c12)C(F)F] Or | 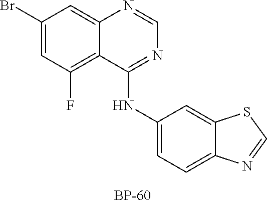 [SMILES: Fc1cc(Br)cc2ncnc(Nc3ccc4ncsc4c3)c12 ; "BP-60"] | | 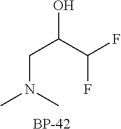 |
| | 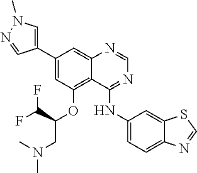 [SMILES: CN(C)C[C@H](Oc1cc(-c2cnn(C)c2)cc2ncnc(Nc3ccc4ncsc4c3)c12)C(F)F] | | | |
| 153 154 | 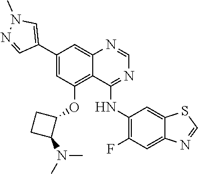 [SMILES: CN(C)[C@H]1CC[C@@H]1Oc1cc(-c2cnn(C)c2)cc2ncnc(Nc3cc4scnc4cc3F)c12] Or | 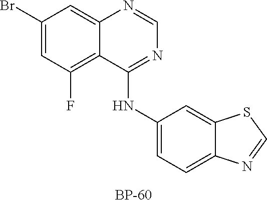 [SMILES: Fc1cc(Br)cc2ncnc(Nc3ccc4ncsc4c3)c12 ; "BP-60"] | | 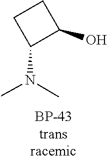 |
| | 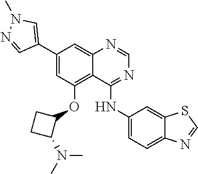 | | | |
| 155 156 | 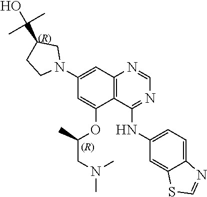 Or | 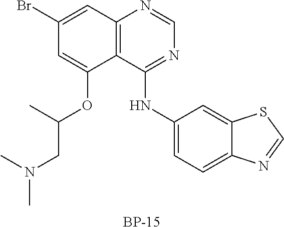 [SMILES: CC(CN(C)C)Oc1cc(Br)cc2ncnc(Nc3ccc4ncsc4c3)c12 ; "BP-15"] | | 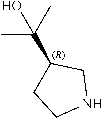 |
| | 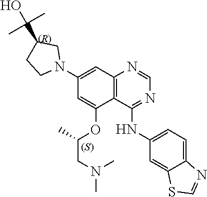 | | | |
| 157 158 | 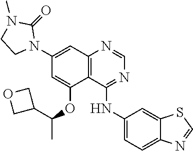 [SMILES: C[C@H](Oc1cc(N2CCN(C)C2=O)cc2ncnc(Nc3ccc4ncsc4c3)c12)C1COC1] Or 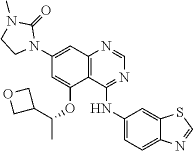 [SMILES: C[C@@H](Oc1cc(N2CCN(C)C2=O)cc2ncnc(Nc3ccc4ncsc4c3)c12)C1COC1] | 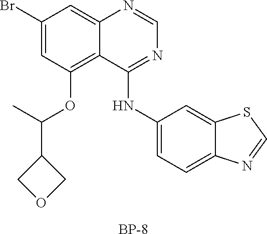 [SMILES: CC(Oc1cc(Br)cc2ncnc(Nc3ccc4ncsc4c3)c12)C1COC1 ; "BP-8"] | | 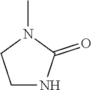 |
| 159 160 | 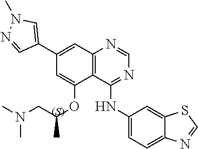 Or 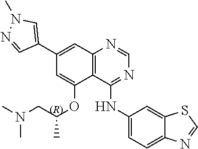 | 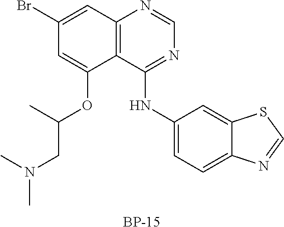 [SMILES: CC(CN(C)C)Oc1cc(Br)cc2ncnc(Nc3ccc4ncsc4c3)c12 ; "BP-15"] | | 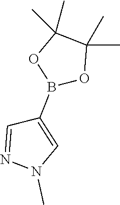 |
| 161 162 | 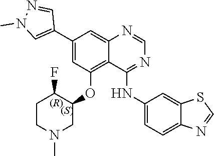 Or 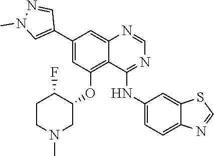 [SMILES: CN1CC[C@H](F)[C@H](Oc2cc(-c3cnn(C)c3)cc3ncnc(Nc4ccc5ncsc5c4)c23)C1] | 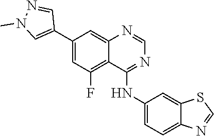 [SMILES: Cn1cc(-c2cc(F)c3c(Nc4ccc5ncsc5c4)ncnc3c2)cn1] BP-67 | | 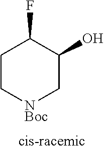 [SMILES: CC(C)(C)OC(=O)N1CC[C@@H](F)[C@@H](O)C1] |
| 163 | 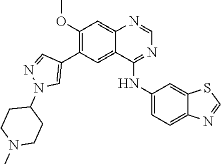 [SMILES: COc1cc2ncnc(Nc3ccc4ncsc4c3)c2cc1-c1cnn(C2CCN(C)CC2)c1] | 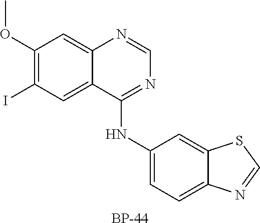 [SMILES: COc1cc2ncnc(Nc3ccc4ncsc4c3)c2cc1I ; "BP-44"] | | 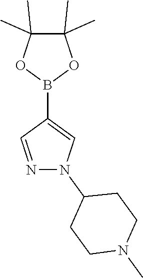 |
| 164 | 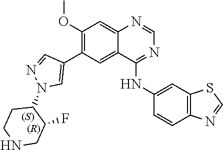 cis-racemic | 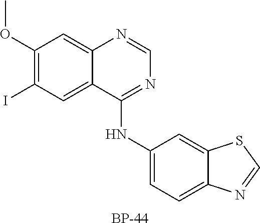 [SMILES: COc1cc2ncnc(Nc3ccc4ncsc4c3)c2cc1I ; "BP-44"] | | 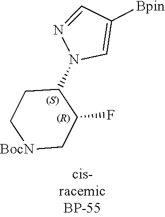 |
| 165 | 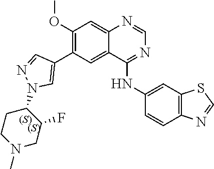 cis-racemic | 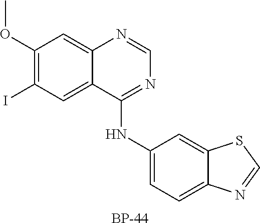 [SMILES: COc1cc2ncnc(Nc3ccc4ncsc4c3)c2cc1I ; "BP-44"] | | 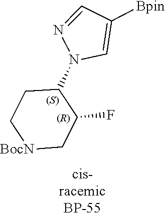 |
| 166 167 | 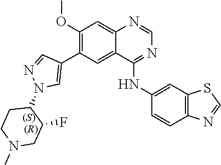 | Example 165 | | |
| | Or | | | |
| | 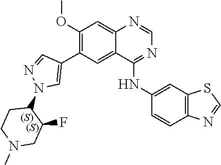 | | | |
| 168 | 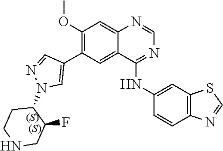 trans-racemic | 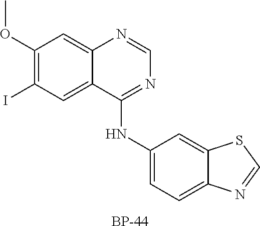 [SMILES: COc1cc2ncnc(Nc3ccc4ncsc4c3)c2cc1I ; "BP-44"] | | 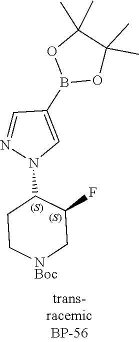 |
| 169 | 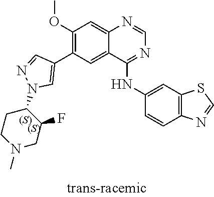 | Example 168 | | |
| 170 171 | 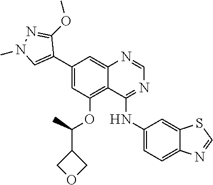 [SMILES: COc1nn(C)cc1-c1cc(O[C@H](C)C2COC2)c2c(Nc3ccc4ncsc4c3)ncnc2c1] Or | 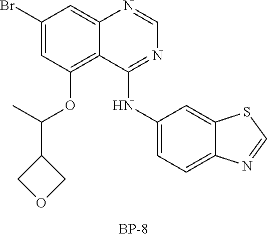 [SMILES: CC(Oc1cc(Br)cc2ncnc(Nc3ccc4ncsc4c3)c12)C1COC1 ; "BP-8"] | | 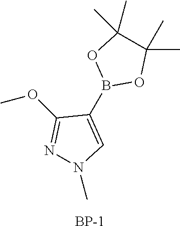 |
| | 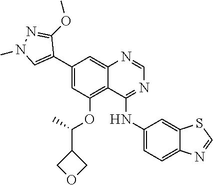 [SMILES: COc1nn(C)cc1-c1cc(O[C@@H](C)C2COC2)c2c(Nc3ccc4ncsc4c3)ncnc2c1] | | | |
| 172 173 | 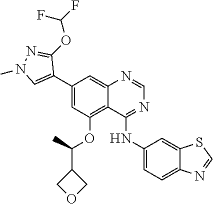 [SMILES: C[C@@H](Oc1cc(-c2cn(C)nc2OC(F)F)cc2ncnc(Nc3ccc4ncsc4c3)c12)C1COC1] Or | 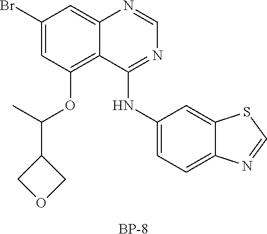 [SMILES: CC(Oc1cc(Br)cc2ncnc(Nc3ccc4ncsc4c3)c12)C1COC1 ; "BP-8"] | | 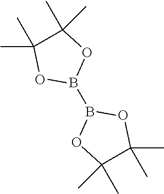 |
| | 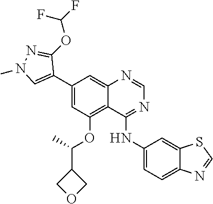 [SMILES: C[C@H](Oc1cc(-c2cn(C)nc2OC(F)F)cc2ncnc(Nc3ccc4ncsc4c3)c12)C1COC1] | | | |
| 174 175 | 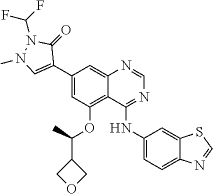 [SMILES: C[C@@H](Oc1cc(-c2cn(C)n(C(F)F)c2=O)cc2ncnc(Nc3ccc4ncsc4c3)c12)C1COC1] Or | 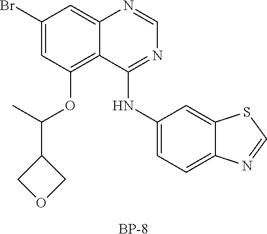 [SMILES: CC(Oc1cc(Br)cc2ncnc(Nc3ccc4ncsc4c3)c12)C1COC1 ; "BP-8"] | | 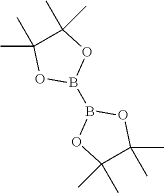 |
| | 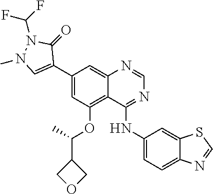 [SMILES: C[C@H](Oc1cc(-c2cn(C)n(C(F)F)c2=O)cc2ncnc(Nc3ccc4ncsc4c3)c12)C1COC1] | | | |
| 176 | 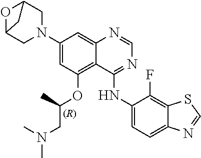 | 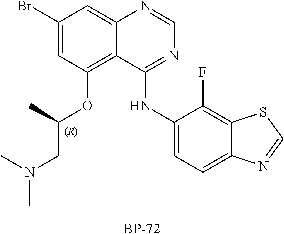 | | 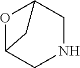 |
| 177 | 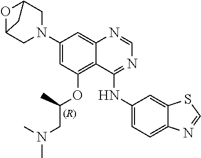 | 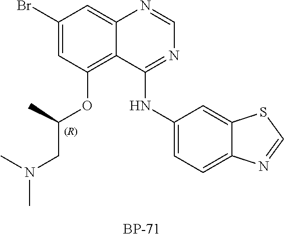 | | 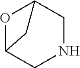 |
| 178 179 | 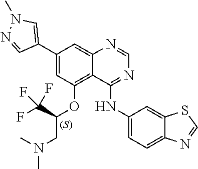 Or | 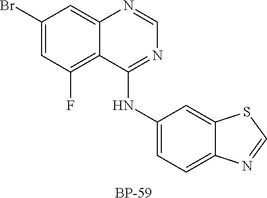 [SMILES: Fc1cc(Br)cc2ncnc(Nc3ccc4ncsc4c3)c12 ; "BP-59"] | | 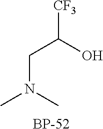 |
| | 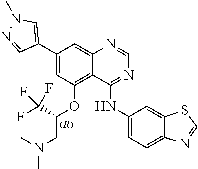 | | | |
| 180 | 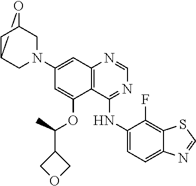 [SMILES: C[C@@H](Oc1cc(N2CC3CC(C2)O3)cc2ncnc(Nc3ccc4ncsc4c3F)c12)C1COC1] | 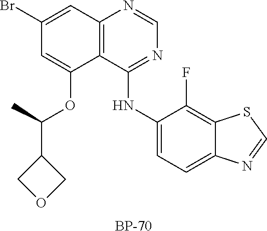 [SMILES: C[C@@H](Oc1cc(Br)cc2ncnc(Nc3ccc4ncsc4c3F)c12)C1COC1 ; "BP-70"] | | 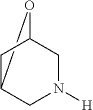 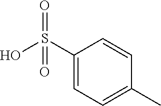 |
| 181 | 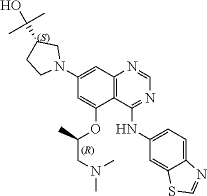 | 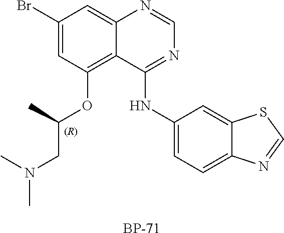 | | 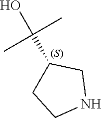 |
| 182 | 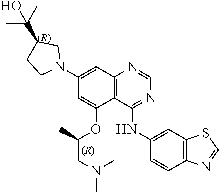 | 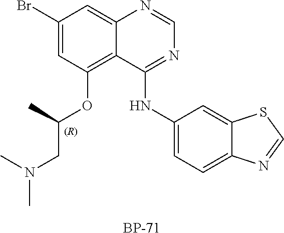 | | 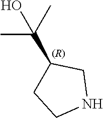 |
| 183 | 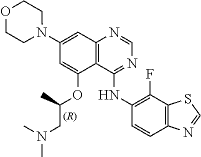 | 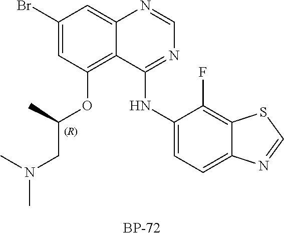 | | 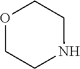 |
| 184 | 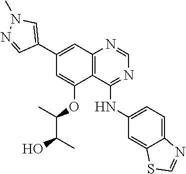 [SMILES: C[C@@H](O)[C@@H](C)Oc1cc(-c2cnn(C)c2)cc2ncnc(Nc3ccc4ncsc4c3)c12] | 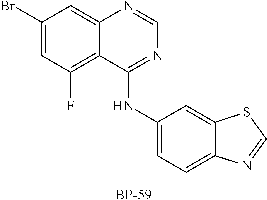 [SMILES: Fc1cc(Br)cc2ncnc(Nc3ccc4ncsc4c3)c12 ; "BP-59"] | | 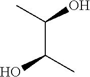 |
| 185 | 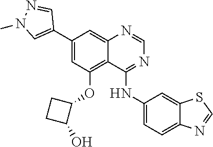 [SMILES: Cn1cc(-c2cc(O[C@H]3CC[C@H]3O)c3c(Nc4ccc5ncsc5c4)ncnc3c2)cn1] | 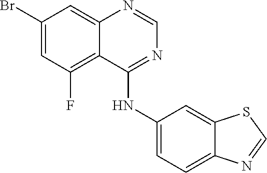 [SMILES: Fc1cc(Br)cc2ncnc(Nc3ccc4ncsc4c3)c12] | | 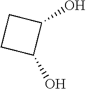 |
| | | BP-59 | | |
| 186 | 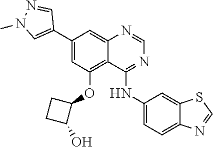 [SMILES: Cn1cc(-c2cc(O[C@@H]3CC[C@H]3O)c3c(Nc4ccc5ncsc5c4)ncnc3c2)cn1] | 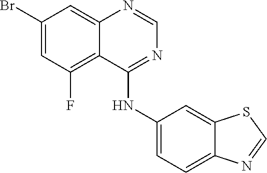 [SMILES: Fc1cc(Br)cc2ncnc(Nc3ccc4ncsc4c3)c12] | | 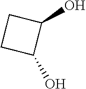 |
| | | BP-59 | | |
| 187 | 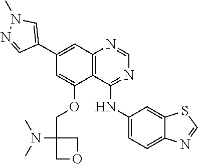 [SMILES: CN(C)C1(COc2cc(-c3cnn(C)c3)cc3ncnc(Nc4ccc5ncsc5c4)c23)COC1] | 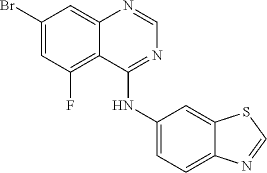 [SMILES: Fc1cc(Br)cc2ncnc(Nc3ccc4ncsc4c3)c12] | | 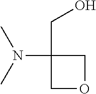 |
| | | BP-59 | | |
| 188 | 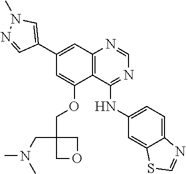 [SMILES: CN(C)CC1(COc2cc(-c3cnn(C)c3)cc3ncnc(Nc4ccc5ncsc5c4)c23)COC1] | 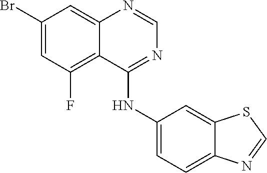 [SMILES: Fc1cc(Br)cc2ncnc(Nc3ccc4ncsc4c3)c12] BP-59 | | 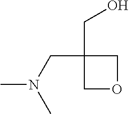 |
| 189 | 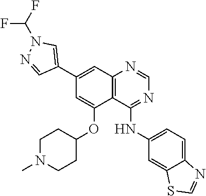 [SMILES: CN1CCC(Oc2cc(-c3cnn(C(F)F)c3)cc3ncnc(Nc4ccc5ncsc5c4)c23)CC1] | 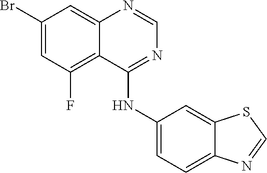 [SMILES: Fc1cc(Br)cc2ncnc(Nc3ccc4ncsc4c3)c12] BP-59 | | 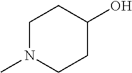 |
| 190 | 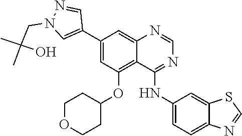 [SMILES: CC(C)(O)Cn1cc(-c2cc(OC3CCOCC3)c3c(Nc4ccc5ncsc5c4)ncnc3c2)cn1] | 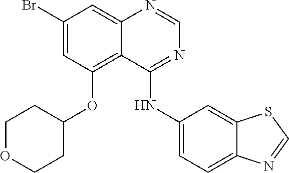 [SMILES: Brc1cc(OC2CCOCC2)c2c(Nc3ccc4ncsc4c3)ncnc2c1] BP-11 | | 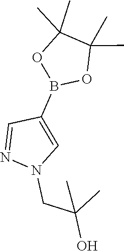 [SMILES: CC(C)(O)Cn1cc(B2OC(C)(C)C(C)(C)O2)cn1] |
| 191 | 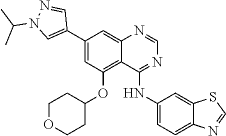 [SMILES: CC(C)n1cc(-c2cc(OC3CCOCC3)c3c(Nc4ccc5ncsc5c4)ncnc3c2)cn1] | 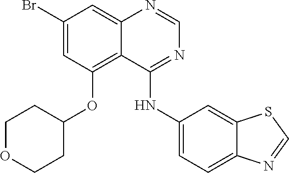 [SMILES: Brc1cc(OC2CCOCC2)c2c(Nc3ccc4ncsc4c3)ncnc2c1] BP-11 | | 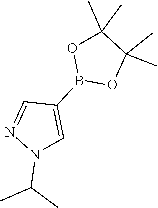 [SMILES: CC(C)n1cc(B2OC(C)(C)C(C)(C)O2)cn1] |
| 192 | 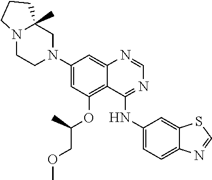 [SMILES: COC[C@@H](C)Oc1cc(N2CCN3CCC[C@]3(C)C2)cc2ncnc(Nc3ccc4ncsc4c3)c12] | 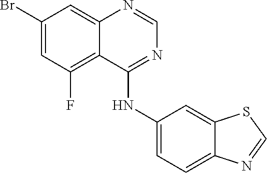 [SMILES: Fc1cc(Br)cc2ncnc(Nc3ccc4ncsc4c3)c12] BP-59 | | 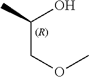 |
| 193 | 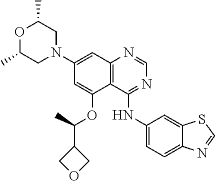 [SMILES: C[C@@H]1CN(c2cc(O[C@H](C)C3COC3)c3c(Nc4ccc5ncsc5c4)ncnc3c2)C[C@H](C)O1] | 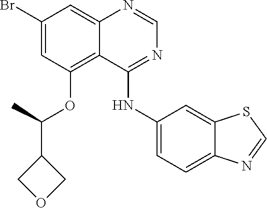 [SMILES: C[C@@H](Oc1cc(Br)cc2ncnc(Nc3ccc4ncsc4c3)c12)C1COC1] | | 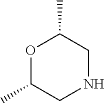 |
| | | BP-70a | | |
| 194 | 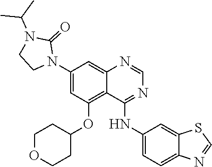 [SMILES: CC(C)N1CCN(c2cc(OC3CCOCC3)c3c(Nc4ccc5ncsc5c4)ncnc3c2)C1=O] | 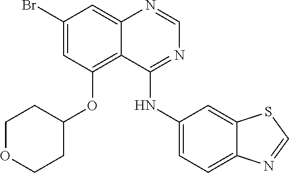 [SMILES: Brc1cc(OC2CCOCC2)c2c(Nc3ccc4ncsc4c3)ncnc2c1] | | 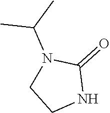 |
| | | BP-11 | | |
| 195 | 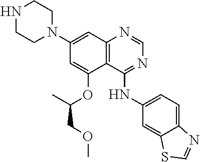 [SMILES: COC[C@@H](C)Oc1cc(N2CCNCC2)cc2ncnc(Nc3ccc4ncsc4c3)c12] | 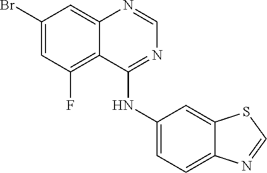 [SMILES: Fc1cc(Br)cc2ncnc(Nc3ccc4ncsc4c3)c12] | | 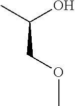 |
| | | BP-59 | | |
| 196 | 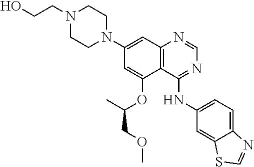 [SMILES: COC[C@@H](C)Oc1cc(N2CCN(CCO)CC2)cc2ncnc(Nc3ccc4ncsc4c3)c12] | 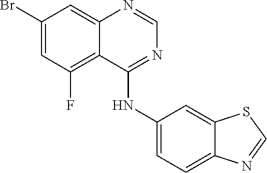 [SMILES: Fc1cc(Br)cc2ncnc(Nc3ccc4ncsc4c3)c12] | | 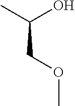 |
| | | BP-59 | | |
| 197 | 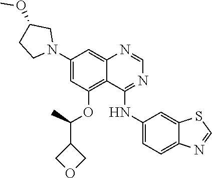 [SMILES: CO[C@H]1CCN(c2cc(O[C@H](C)C3COC3)c3c(Nc4ccc5ncsc5c4)ncnc3c2)C1] | 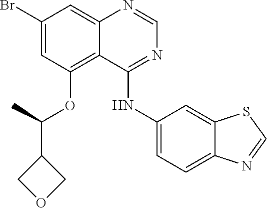 [SMILES: C[C@@H](Oc1cc(Br)cc2ncnc(Nc3ccc4ncsc4c3)c12)C1COC1] | | 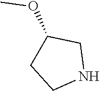 |
| | | BP-70a | | |
| 198 | 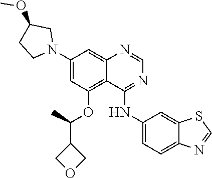 [SMILES: CO[C@@H]1CCN(c2cc(O[C@H](C)C3COC3)c3c(Nc4ccc5ncsc5c4)ncnc3c2)C1] | 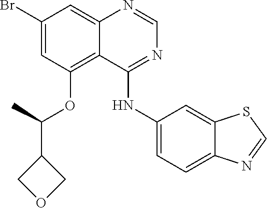 [SMILES: C[C@@H](Oc1cc(Br)cc2ncnc(Nc3ccc4ncsc4c3)c12)C1COC1] | | 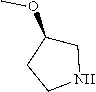 |
| | | BP-70a | | |
| 199 | 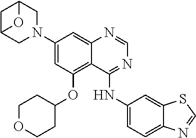 [SMILES: c1nc(Nc2ccc3ncsc3c2)c2c(OC3CCOCC3)cc(N3CC4CC(C3)O4)cc2n1] | 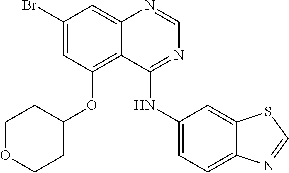 [SMILES: Brc1cc(OC2CCOCC2)c2c(Nc3ccc4ncsc4c3)ncnc2c1] | | 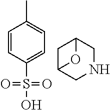 |
| | | BP-11 | | |
| 200 | 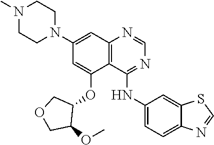 [SMILES: CO[C@H]1COC[C@@H]1Oc1cc(N2CCN(C)CC2)cc2ncnc(Nc3ccc4ncsc4c3)c12] | 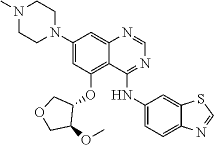 [SMILES: CO[C@H]1COC[C@@H]1Oc1cc(N2CCN(C)CC2)cc2ncnc(Nc3ccc4ncsc4c3)c12] | | 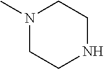 |
| | | BP-51 | | |
| 201 | 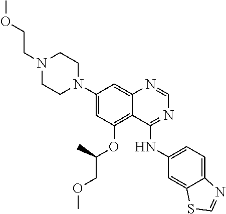 [SMILES: COCCN1CCN(c2cc(O[C@H](C)COC)c3c(Nc4ccc5ncsc5c4)ncnc3c2)CC1] | 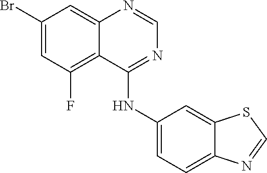 [SMILES: Fc1cc(Br)cc2ncnc(Nc3ccc4ncsc4c3)c12] BP-59 | | 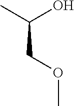 |
| 202 | 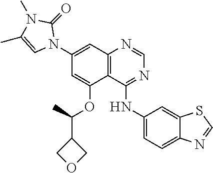 [SMILES: Cc1cn(-c2cc(O[C@H](C)C3COC3)c3c(Nc4ccc5ncsc5c4)ncnc3c2)c(=O)n1C] | 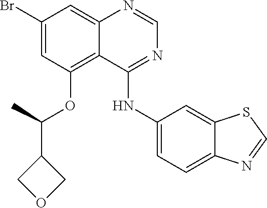 [SMILES: C[C@@H](Oc1cc(Br)cc2ncnc(Nc3ccc4ncsc4c3)c12)C1COC1] | | 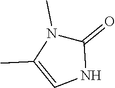 |
| | | BP-70a | | |
| 203 | 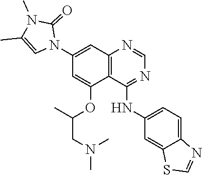 [SMILES: Cc1cn(-c2cc(OC(C)CN(C)C)c3c(Nc4ccc5ncsc5c4)ncnc3c2)c(=O)n1C] | 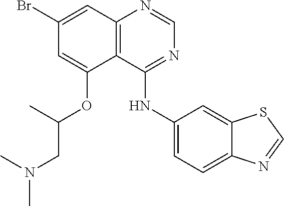 [SMILES: CC(CN(C)C)Oc1cc(Br)cc2ncnc(Nc3ccc4ncsc4c3)c12] | | 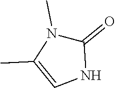 |
| | | BP-15 | | |
| 204 | 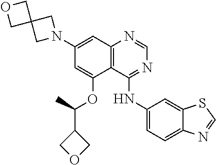 [SMILES: C[C@@H](Oc1cc(N2CC3(COC3)C2)cc2ncnc(Nc3ccc4ncsc4c3)c12)C1COC1] | 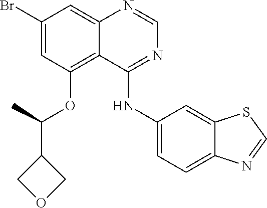 [SMILES: C[C@@H](Oc1cc(Br)cc2ncnc(Nc3ccc4ncsc4c3)c12)C1COC1] | | 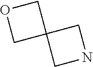 |
| | | BP-70a | | |
| 205 206 | 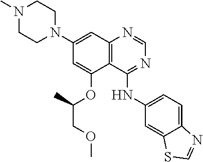 [SMILES: COC[C@@H](C)Oc1cc(N2CCN(C)CC2)cc2ncnc(Nc3ccc4ncsc4c3)c12] OR | 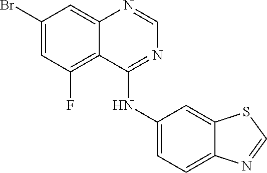 [SMILES: Fc1cc(Br)cc2ncnc(Nc3ccc4ncsc4c3)c12] BP-59 | | 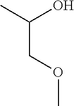 |
| | 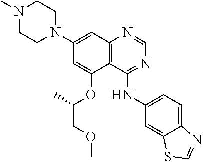 [SMILES: COC[C@H](C)Oc1cc(N2CCN(C)CC2)cc2ncnc(Nc3ccc4ncsc4c3)c12] | | | |
| 207 208 | 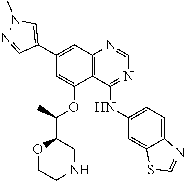 [SMILES: C[C@@H](Oc1cc(-c2cnn(C)c2)cc2ncnc(Nc3ccc4ncsc4c3)c12)[C@H]1CNCCO1] Or | 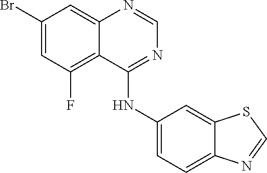 [SMILES: Fc1cc(Br)cc2ncnc(Nc3ccc4ncsc4c3)c12] BP-59 | | 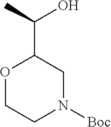 [SMILES: C[C@@H](O)C1CN(C(=O)OC(C)(C)C)CCO1] |
| | 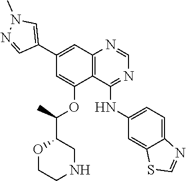 [SMILES: C[C@@H](Oc1cc(-c2cnn(C)c2)cc2ncnc(Nc3ccc4ncsc4c3)c12)[C@@H]1CNCCO1] | | | |
| 209 210 | 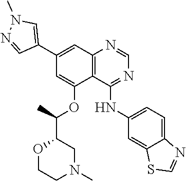 [SMILES: C[C@@H](Oc1cc(-c2cnn(C)c2)cc2ncnc(Nc3ccc4ncsc4c3)c12)[C@@H]1CN(C)CCO1] OR | 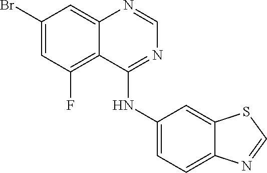 [SMILES: Fc1cc(Br)cc2ncnc(Nc3ccc4ncsc4c3)c12] BP-59 | | 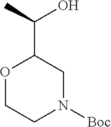 [SMILES: C[C@@H](O)C1CN(C(=O)OC(C)(C)C)CCO1] |
| | 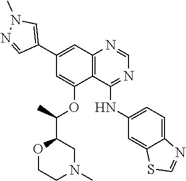 [SMILES: C[C@@H](Oc1cc(-c2cnn(C)c2)cc2ncnc(Nc3ccc4ncsc4c3)c12)[C@H]1CN(C)CCO1] | | | |
| 211 212 | 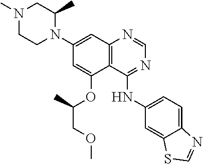 [SMILES: COC[C@@H](C)Oc1cc(N2CCN(C)C[C@H]2C)cc2ncnc(Nc3ccc4ncsc4c3)c12] OR | 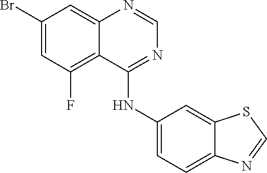 [SMILES: Fc1cc(Br)cc2ncnc(Nc3ccc4ncsc4c3)c12] BP-59 | | 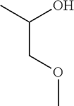 |
| | 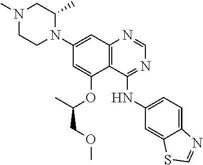 [SMILES: COC[C@@H](C)Oc1cc(N2CCN(C)C[C@@H]2C)cc2ncnc(Nc3ccc4ncsc4c3)c12] | | | |
| 226 | 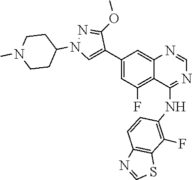 [SMILES: COc1nn(C2CCN(C)CC2)cc1-c1cc(F)c2c(Nc3ccc4ncsc4c3F)ncnc2c1] | 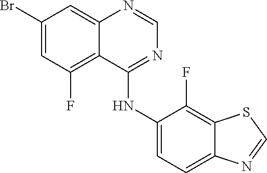 [SMILES: Fc1c(Nc2ncnc3cc(Br)cc(F)c23)ccc2ncsc12] BP-59 | | 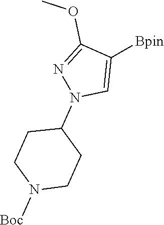 |
| | | | | BP-17 |
| 227 | 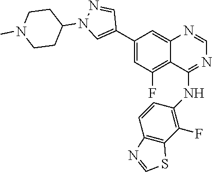 [SMILES: CN1CCC(n2cc(-c3cc(F)c4c(Nc5ccc6ncsc6c5F)ncnc4c3)cn2)CC1] | 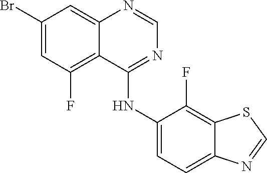 [SMILES: Fc1c(Nc2ncnc3cc(Br)cc(F)c23)ccc2ncsc12] | | 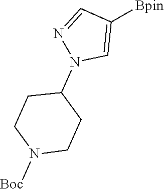 |
| | | BP-59 | | |
| 240 | 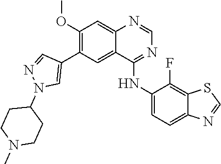 [SMILES: COc1cc2ncnc(Nc3ccc4ncsc4c3F)c2cc1-c1cnn(C2CCN(C)CC2)c1] | 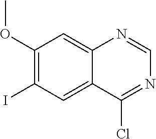 | | 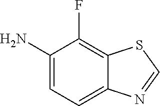 [SMILES: Nc1ccc2ncsc2c1F] BP-23 |
| 241 | 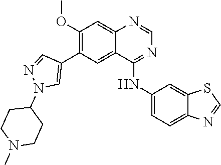 [SMILES: COc1cc2ncnc(Nc3ccc4ncsc4c3)c2cc1-c1cnn(C2CCN(C)CC2)c1] | 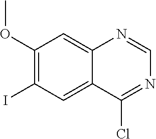 | | 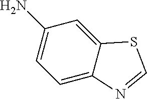 |
| 242 | 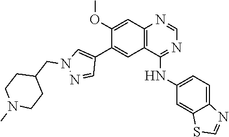 [SMILES: COc1cc2ncnc(Nc3ccc4ncsc4c3)c2cc1-c1cnn(CC2CCN(C)CC2)c1] | 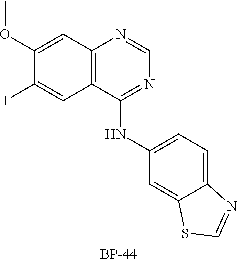 [SMILES: COc1cc2ncnc(Nc3ccc4ncsc4c3)c2cc1I ; "BP-44"] | | 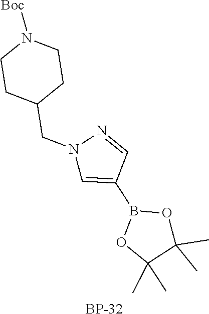 [SMILES: CC(C)(C)OC(=O)N1CCC(Cn2cc(B3OC(C)(C)C(C)(C)O3)cn2)CC1 ; "BP-32"] |
| 243 | 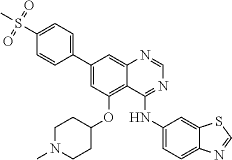 [SMILES: CN1CCC(Oc2cc(-c3ccc(S(C)(=O)=O)cc3)cc3ncnc(Nc4ccc5ncsc5c4)c23)CC1] | 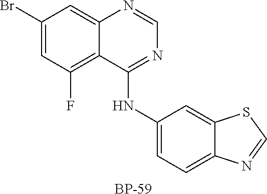 [SMILES: Fc1cc(Br)cc2ncnc(Nc3ccc4ncsc4c3)c12 ; "BP-59"] | | 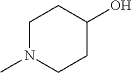 |
| 245 | 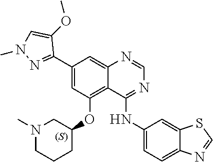 | 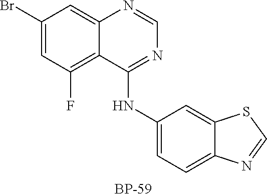 [SMILES: Fc1cc(Br)cc2ncnc(Nc3ccc4ncsc4c3)c12 ; "BP-59"] | | 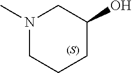 |
| 246 | 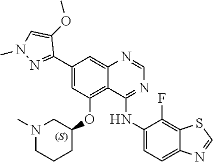 | 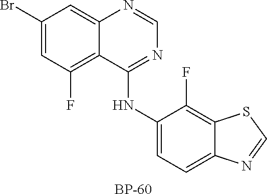 [SMILES: Fc1c(Nc2ncnc3cc(Br)cc(F)c23)ccc2ncsc12 ; "BP-60"] | | 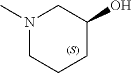 |
| 247 | 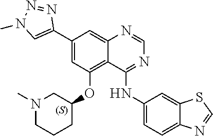 | 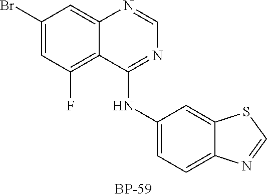 [SMILES: Fc1cc(Br)cc2ncnc(Nc3ccc4ncsc4c3)c12 ; "BP-59"] | | 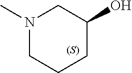 |
| 248 | 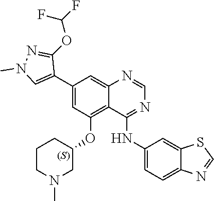 | 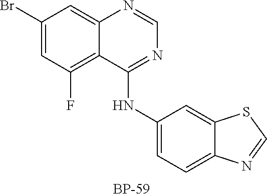 [SMILES: Fc1cc(Br)cc2ncnc(Nc3ccc4ncsc4c3)c12 ; "BP-59"] | | 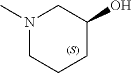 |
| 249 | 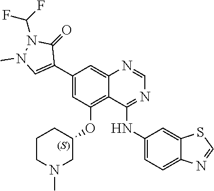 | 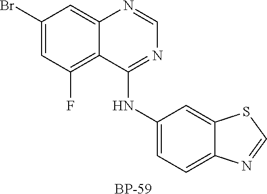 [SMILES: Fc1cc(Br)cc2ncnc(Nc3ccc4ncsc4c3)c12 ; "BP-59"] | | 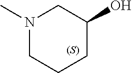 |
| 250 | 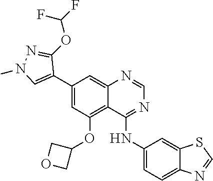 [SMILES: Cn1cc(-c2cc(OC3COC3)c3c(Nc4ccc5ncsc5c4)ncnc3c2)c(OC(F)F)n1] | 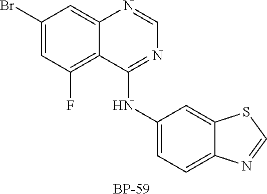 [SMILES: Fc1cc(Br)cc2ncnc(Nc3ccc4ncsc4c3)c12 ; "BP-59"] | | 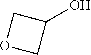 |
| 251 | 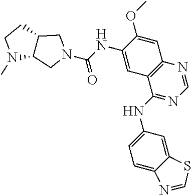 [SMILES: COc1cc2ncnc(Nc3ccc4ncsc4c3)c2cc1NC(=O)N1C[C@H]2CCN(C)[C@H]2C1] | 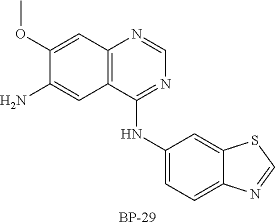 [SMILES: COc1cc2ncnc(Nc3ccc4ncsc4c3)c2cc1N ; "BP-29"] | | 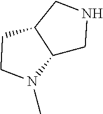 |
| 252 | 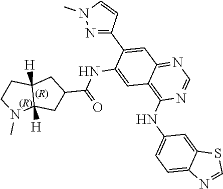 | 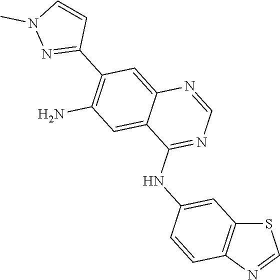 [SMILES: Cn1ccc(-c2cc3ncnc(Nc4ccc5ncsc5c4)c3cc2N)n1] | | 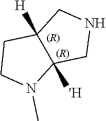 |
| 253 | 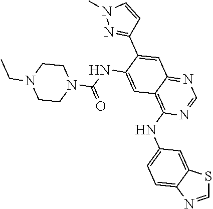 [SMILES: CCN1CCN(C(=O)Nc2cc3c(Nc4ccc5ncsc5c4)ncnc3cc2-c2ccn(C)n2)CC1] | 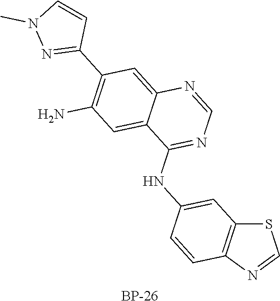 [SMILES: Cn1ccc(-c2cc3ncnc(Nc4ccc5ncsc5c4)c3cc2N)n1 ; "BP-26"] | | 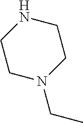 |
| 254 | 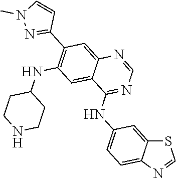 [SMILES: Cn1ccc(-c2cc3ncnc(Nc4ccc5ncsc5c4)c3cc2NC2CCNCC2)n1] | 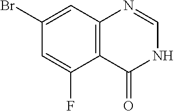 [SMILES: O=c1[nH]cnc2cc(Br)cc(F)c12] BP-28 | | 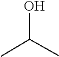 |
| 255 | 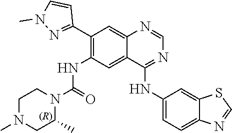 | 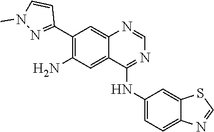 [SMILES: Cn1ccc(-c2cc3ncnc(Nc4ccc5ncsc5c4)c3cc2N)n1] BP-26 | | 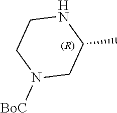 |
| 256 | 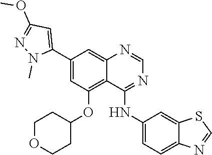 [SMILES: COc1cc(-c2cc(OC3CCOCC3)c3c(Nc4ccc5ncsc5c4)ncnc3c2)n(C)n1] | 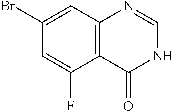 [SMILES: O=c1[nH]cnc2cc(Br)cc(F)c12] BP-28 | | 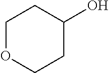 Merthod A1 |
| 257 | 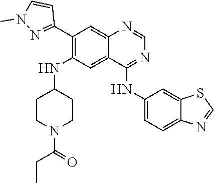 [SMILES: CCC(=O)N1CCC(Nc2cc3c(Nc4ccc5ncsc5c4)ncnc3cc2-c2ccn(C)n2)CC1] | 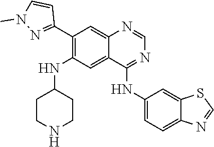 [SMILES: Cn1ccc(-c2cc3ncnc(Nc4ccc5ncsc5c4)c3cc2NC2CCNCC2)n1] BP-30 | | 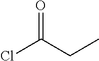 |
| 258 | 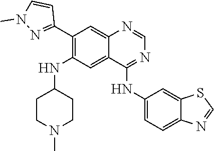 [SMILES: CN1CCC(Nc2cc3c(Nc4ccc5ncsc5c4)ncnc3cc2-c2ccn(C)n2)CC1] | 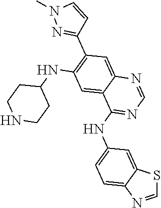 [SMILES: Cn1ccc(-c2cc3ncnc(Nc4ccc5ncsc5c4)c3cc2NC2CCNCC2)n1] | | — |
| | | BP-30 | | |
| 259 | 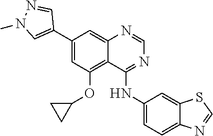 [SMILES: Cn1cc(-c2cc(OC3CC3)c3c(Nc4ccc5ncsc5c4)ncnc3c2)cn1] | 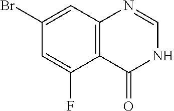 [SMILES: O=c1[nH]cnc2cc(Br)cc(F)c12] BP-28 | | 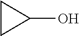 |
| 260 | 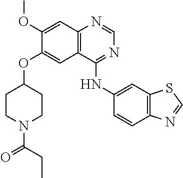 [SMILES: CCC(=O)N1CCC(Oc2cc3c(Nc4ccc5ncsc5c4)ncnc3cc2OC)CC1] | 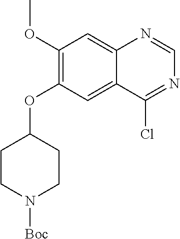 [SMILES: COc1cc2ncnc(Cl)c2cc1OC1CCN(C(=O)OC(C)(C)C)CC1] | | 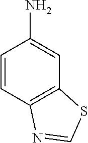 |
| | | BP-19 | | |
| 261 | 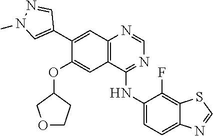 [SMILES: Cn1cc(-c2cc3ncnc(Nc4ccc5ncsc5c4F)c3cc2OC2CCOC2)cn1] | 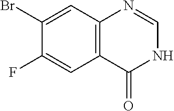 [SMILES: O=c1[nH]cnc2cc(Br)c(F)cc12] BP-45a | | 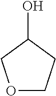 |
| 262 | 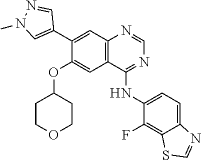 [SMILES: Cn1cc(-c2cc3ncnc(Nc4ccc5ncsc5c4F)c3cc2OC2CCOCC2)cn1] | 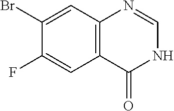 [SMILES: O=c1[nH]cnc2cc(Br)c(F)cc12] BP-45a | | 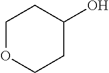 |
| 263 | 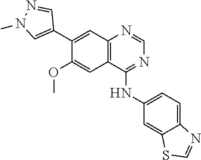 [SMILES: COc1cc2c(Nc3ccc4ncsc4c3)ncnc2cc1-c1cnn(C)c1] | 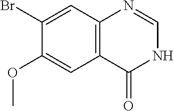 | | 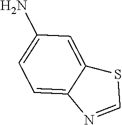 |
| 264 | 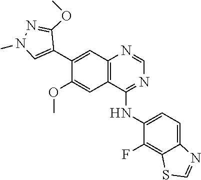 [SMILES: COc1cc2c(Nc3ccc4ncsc4c3F)ncnc2cc1-c1cn(C)nc1OC] | 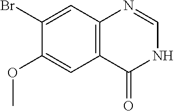 | | 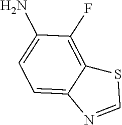 [SMILES: Nc1ccc2ncsc2c1F] BP-23 |
| 265 | 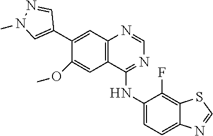 [SMILES: COc1cc2c(Nc3ccc4ncsc4c3F)ncnc2cc1-c1cnn(C)c1] | 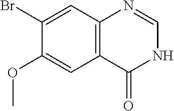 | | 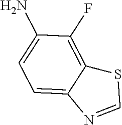 [SMILES: Nc1ccc2ncsc2c1F] |
| | | | | BP-23 |
| 266 | 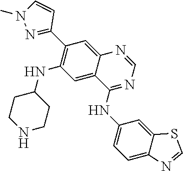 [SMILES: Cn1ccc(-c2cc3ncnc(Nc4ccc5ncsc5c4)c3cc2NC2CCNCC2)n1] | Int BP-30 = Example 266 | | |
| | Int BP-30 | | | |
| 270 | 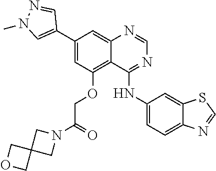 [SMILES: Cn1cc(-c2cc(OCC(=O)N3CC4(COC4)C3)c3c(Nc4ccc5ncsc5c4)ncnc3c2)cn1] | 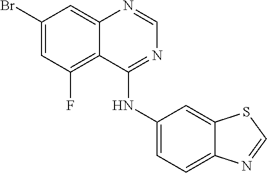 [SMILES: Fc1cc(Br)cc2ncnc(Nc3ccc4ncsc4c3)c12] | | 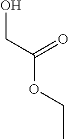 |
| | | BP-59 | | |
| 271 | 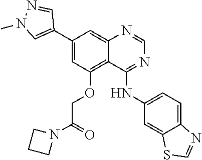 [SMILES: Cn1cc(-c2cc(OCC(=O)N3CCC3)c3c(Nc4ccc5ncsc5c4)ncnc3c2)cn1] | 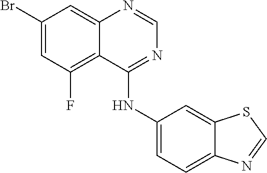 [SMILES: Fc1cc(Br)cc2ncnc(Nc3ccc4ncsc4c3)c12] | | 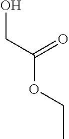 |
| | | BP-59 | | |
| 272 | 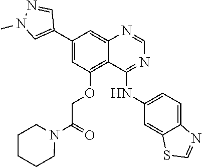 [SMILES: Cn1cc(-c2cc(OCC(=O)N3CCCCC3)c3c(Nc4ccc5ncsc5c4)ncnc3c2)cn1] | 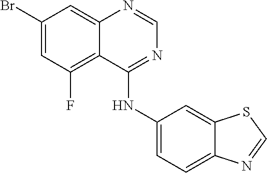 [SMILES: Fc1cc(Br)cc2ncnc(Nc3ccc4ncsc4c3)c12] | | 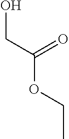 |
| | | BP-59 | | |
| 273 | 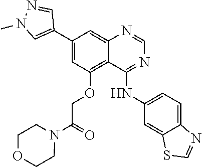 [SMILES: Cn1cc(-c2cc(OCC(=O)N3CCOCC3)c3c(Nc4ccc5ncsc5c4)ncnc3c2)cn1] | 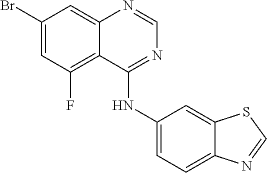 [SMILES: Fc1cc(Br)cc2ncnc(Nc3ccc4ncsc4c3)c12] | | 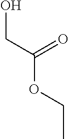 |
| | | BP-59 | | |
| 274 | 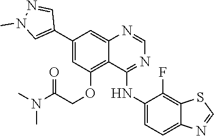 [SMILES: CN(C)C(=O)COc1cc(-c2cnn(C)c2)cc2ncnc(Nc3ccc4ncsc4c3F)c12] | 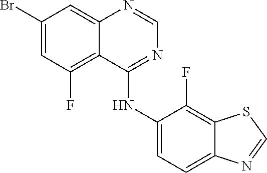 [SMILES: Fc1c(Nc2ncnc3cc(Br)cc(F)c23)ccc2ncsc12] | | 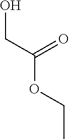 |
| | | BP-60 | | |
| 275 | 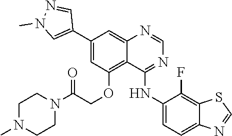 [SMILES: CN1CCN(C(=O)COc2cc(-c3cnn(C)c3)cc3ncnc(Nc4ccc5ncsc5c4F)c23)CC1] | 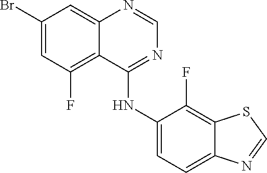 [SMILES: Fc1c(Nc2ncnc3cc(Br)cc(F)c23)ccc2ncsc12] | | 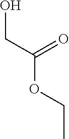 |
| | | BP-60 | | |
| 276 | 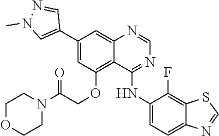 [SMILES: Cn1cc(-c2cc(OCC(=O)N3CCOCC3)c3c(Nc4ccc5ncsc5c4F)ncnc3c2)cn1] | 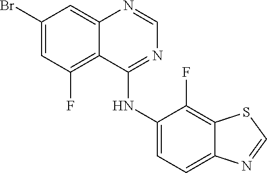 [SMILES: Fc1c(Nc2ncnc3cc(Br)cc(F)c23)ccc2ncsc12] | | 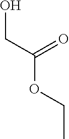 |
| | | BP-60 | | |
| 277 | 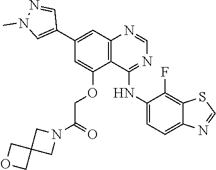 [SMILES: Cn1cc(-c2cc(OCC(=O)N3CC4(COC4)C3)c3c(Nc4ccc5ncsc5c4F)ncnc3c2)cn1] | 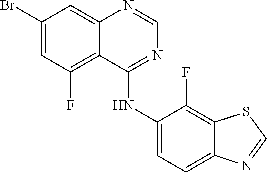 [SMILES: Fc1c(Nc2ncnc3cc(Br)cc(F)c23)ccc2ncsc12] | | 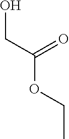 |
| | | BP-60 | | |
| 278 | 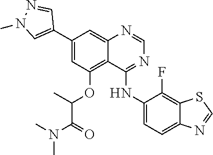 [SMILES: CC(Oc1cc(-c2cnn(C)c2)cc2ncnc(Nc3ccc4ncsc4c3F)c12)C(=O)N(C)C] | 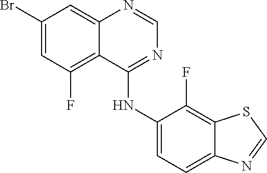 [SMILES: Fc1c(Nc2ncnc3cc(Br)cc(F)c23)ccc2ncsc12] | | |
| | | BP-60 | | |
| 279 280 | 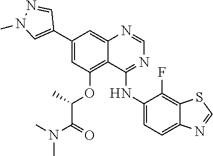 [SMILES: C[C@H](Oc1cc(-c2cnn(C)c2)cc2ncnc(Nc3ccc4ncsc4c3F)c12)C(=O)N(C)C] | Example 278 | | |
| | 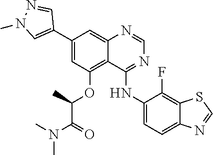 [SMILES: C[C@@H](Oc1cc(-c2cnn(C)c2)cc2ncnc(Nc3ccc4ncsc4c3F)c12)C(=O)N(C)C] | | | |
| 281 | 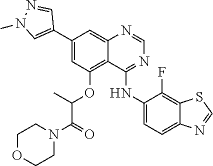 [SMILES: CC(Oc1cc(-c2cnn(C)c2)cc2ncnc(Nc3ccc4ncsc4c3F)c12)C(=O)N1CCOCC1] | 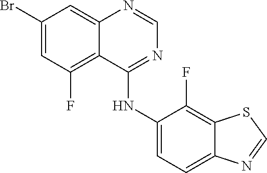 [SMILES: Fc1c(Nc2ncnc3cc(Br)cc(F)c23)ccc2ncsc12] | | 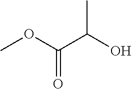 |
| | | BP-60 | | |
| 282 283 | 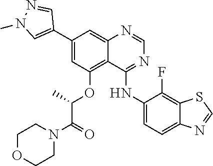 [SMILES: C[C@H](Oc1cc(-c2cnn(C)c2)cc2ncnc(Nc3ccc4ncsc4c3F)c12)C(=O)N1CCOCC1] | Example 281 | | |
| | OR | | | |
| | 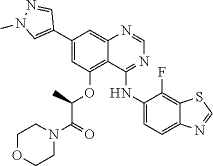 [SMILES: C[C@@H](Oc1cc(-c2cnn(C)c2)cc2ncnc(Nc3ccc4ncsc4c3F)c12)C(=O)N1CCOCC1] | | | |
| 284 | 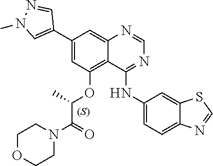 | 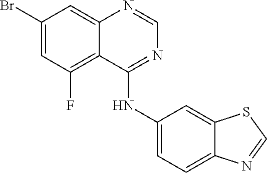 [SMILES: Fc1cc(Br)cc2ncnc(Nc3ccc4ncsc4c3)c12] | | 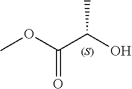 |
| | | BP-59 | | |
| 285 | 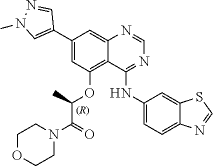 | 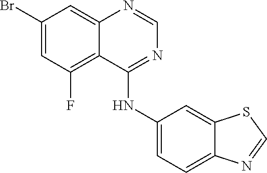 [SMILES: Fc1cc(Br)cc2ncnc(Nc3ccc4ncsc4c3)c12] | | 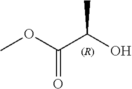 |
| | | BP-59 | | |
| 286 | 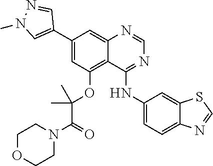 [SMILES: Cn1cc(-c2cc(OC(C)(C)C(=O)N3CCOCC3)c3c(Nc4ccc5ncsc5c4)ncnc3c2)cn1] | 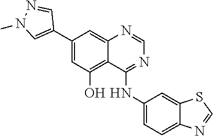 [SMILES: Cn1cc(-c2cc(O)c3c(Nc4ccc5ncsc5c4)ncnc3c2)cn1] BP-62 | | 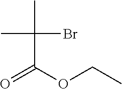 |
| 287 288 | 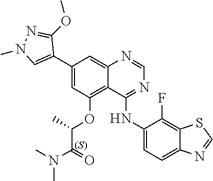 OR | 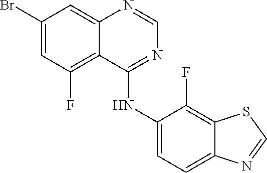 [SMILES: Fc1c(Nc2ncnc3cc(Br)cc(F)c23)ccc2ncsc12] BP-60 | | 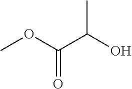 |
| | 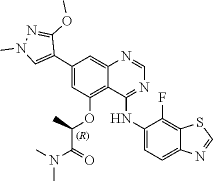 | | | |
| 289 290 | 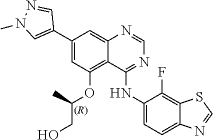 OR 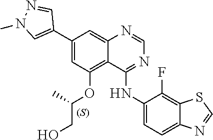 | 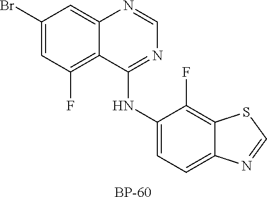 [SMILES: Fc1c(Nc2ncnc3cc(Br)cc(F)c23)ccc2ncsc12 ; "BP-60"] | | 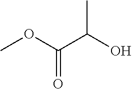 |
| 291 292 | 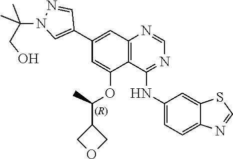 OR 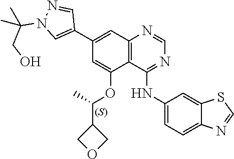 | 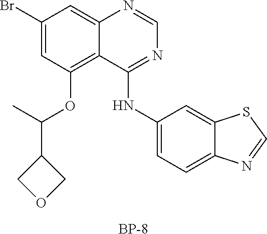 [SMILES: CC(Oc1cc(Br)cc2ncnc(Nc3ccc4ncsc4c3)c12)C1COC1 ; "BP-8"] | | 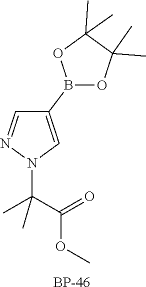 |
| 293 294 | 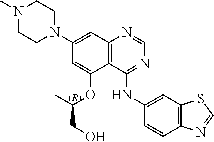 OR 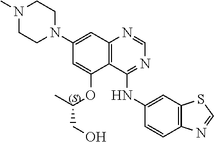 | 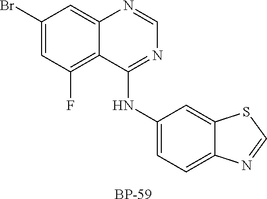 [SMILES: Fc1cc(Br)cc2ncnc(Nc3ccc4ncsc4c3)c12 ; "BP-59"] | | 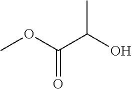 |
| 295 296 | 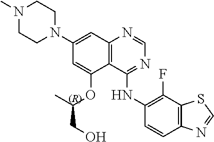 OR 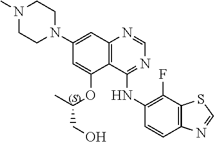 | 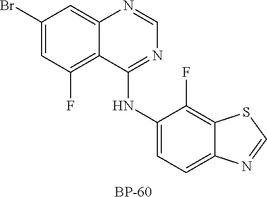 [SMILES: Fc1c(Nc2ncnc3cc(Br)cc(F)c23)ccc2ncsc12 ; "BP-60"] | | 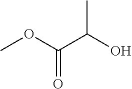 |
| 297 298 | 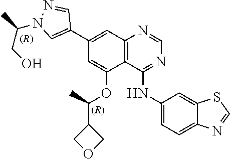 OR 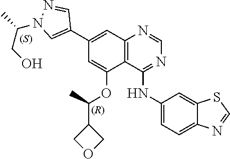 | 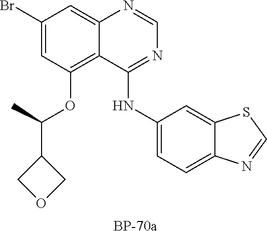 [SMILES: C[C@@H](Oc1cc(Br)cc2ncnc(Nc3ccc4ncsc4c3)c12)C1COC1 ; "BP-70a"] | | 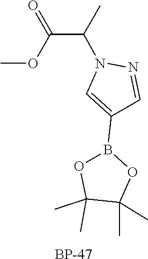 |
| 299 300 | 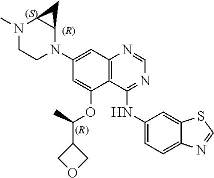 OR | 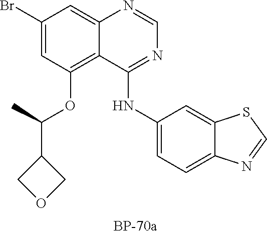 [SMILES: C[C@@H](Oc1cc(Br)cc2ncnc(Nc3ccc4ncsc4c3)c12)C1COC1 ; "BP-70a"] | | 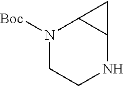 [SMILES: CC(C)(C)OC(=O)N1CCNC2CC21] |
| | 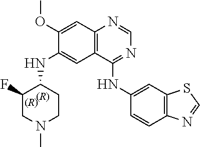 | | | |
| 301 | 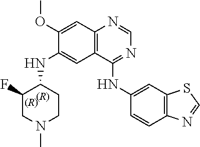 | 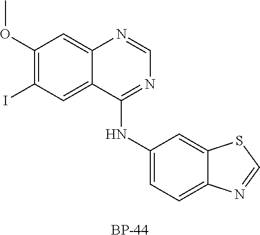 [SMILES: COc1cc2ncnc(Nc3ccc4ncsc4c3)c2cc1I ; "BP-44"] | | 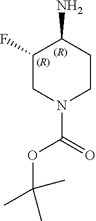 |
| 305 | 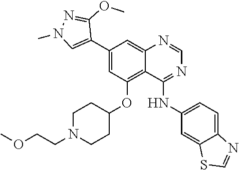 [SMILES: COCCN1CCC(Oc2cc(-c3cn(C)nc3OC)cc3ncnc(Nc4ccc5ncsc5c4)c23)CC1] | 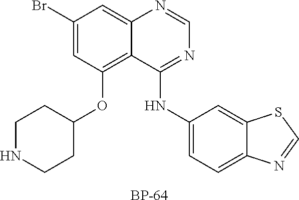 [SMILES: Brc1cc(OC2CCNCC2)c2c(Nc3ccc4ncsc4c3)ncnc2c1 ; "BP-64"] | | 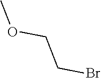 |
| 306 | 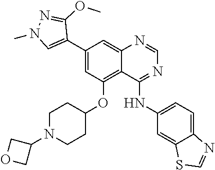 [SMILES: COc1nn(C)cc1-c1cc(OC2CCN(C3COC3)CC2)c2c(Nc3ccc4ncsc4c3)ncnc2c1] | 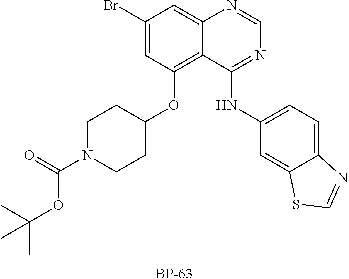 [SMILES: CC(C)(C)OC(=O)N1CCC(Oc2cc(Br)cc3ncnc(Nc4ccc5ncsc5c4)c23)CC1 ; "BP-63"] | | 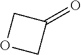 |
| 307 | 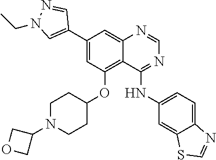 [SMILES: CCn1cc(-c2cc(OC3CCN(C4COC4)CC3)c3c(Nc4ccc5ncsc5c4)ncnc3c2)cn1] | 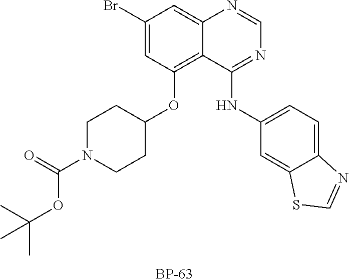 [SMILES: CC(C)(C)OC(=O)N1CCC(Oc2cc(Br)cc3ncnc(Nc4ccc5ncsc5c4)c23)CC1 ; "BP-63"] | | 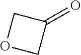 |
| 308 | 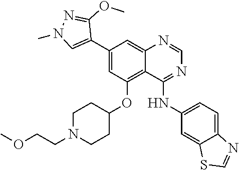 [SMILES: COCCN1CCC(Oc2cc(-c3cn(C)nc3OC)cc3ncnc(Nc4ccc5ncsc5c4)c23)CC1] | 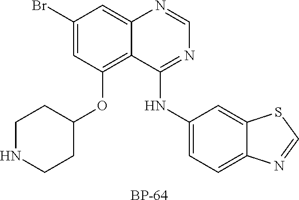 [SMILES: Brc1cc(OC2CCNCC2)c2c(Nc3ccc4ncsc4c3)ncnc2c1 ; "BP-64"] | | 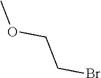 |
| 309 | 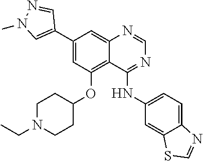 [SMILES: CCN1CCC(Oc2cc(-c3cnn(C)c3)cc3ncnc(Nc4ccc5ncsc5c4)c23)CC1] | 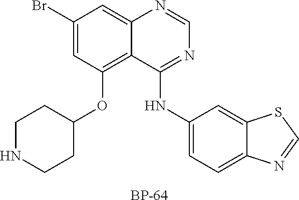 [SMILES: Brc1cc(OC2CCNCC2)c2c(Nc3ccc4ncsc4c3)ncnc2c1 ; "BP-64"] | | 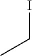 |
| 310 | 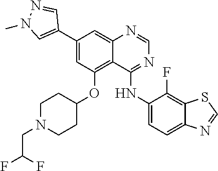 [SMILES: Cn1cc(-c2cc(OC3CCN(CC(F)F)CC3)c3c(Nc4ccc5ncsc5c4F)ncnc3c2)cn1] | 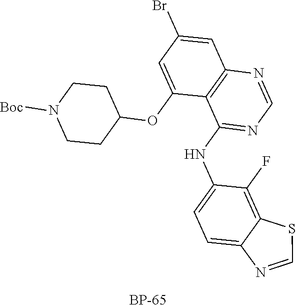 [SMILES: CC(C)(C)OC(=O)N1CCC(Oc2cc(Br)cc3ncnc(Nc4ccc5ncsc5c4F)c23)CC1 ; "BP-65"] | | 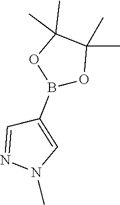 |
| 311 | 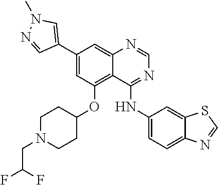 [SMILES: Cn1cc(-c2cc(OC3CCN(CC(F)F)CC3)c3c(Nc4ccc5ncsc5c4)ncnc3c2)cn1] | 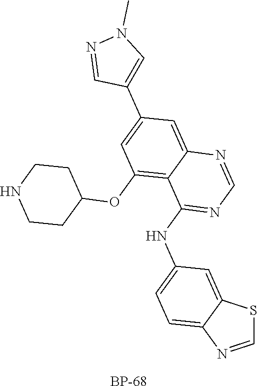 [SMILES: Cn1cc(-c2cc(OC3CCNCC3)c3c(Nc4ccc5ncsc5c4)ncnc3c2)cn1 ; "BP-68"] | | 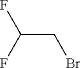 |
| 312 | 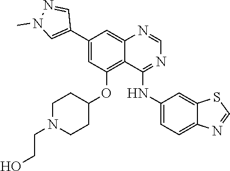 [SMILES: Cn1cc(-c2cc(OC3CCN(CCO)CC3)c3c(Nc4ccc5ncsc5c4)ncnc3c2)cn1] | 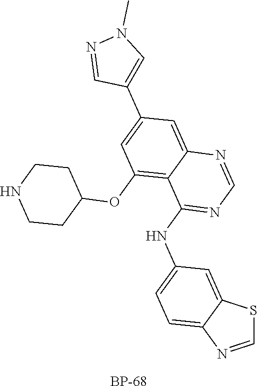 [SMILES: Cn1cc(-c2cc(OC3CCNCC3)c3c(Nc4ccc5ncsc5c4)ncnc3c2)cn1 ; "BP-68"] | | 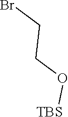 |
| 313 | 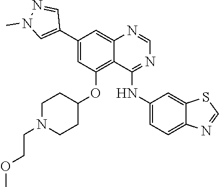 [SMILES: COCCN1CCC(Oc2cc(-c3cnn(C)c3)cc3ncnc(Nc4ccc5ncsc5c4)c23)CC1] | 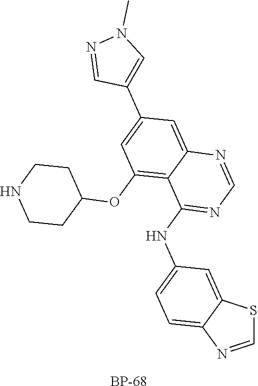 [SMILES: Cn1cc(-c2cc(OC3CCNCC3)c3c(Nc4ccc5ncsc5c4)ncnc3c2)cn1 ; "BP-68"] | | 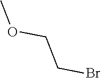 |
| 314 | 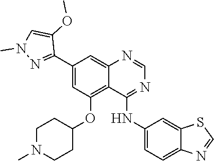 [SMILES: COc1cn(C)nc1-c1cc(OC2CCN(C)CC2)c2c(Nc3ccc4ncsc4c3)ncnc2c1] | 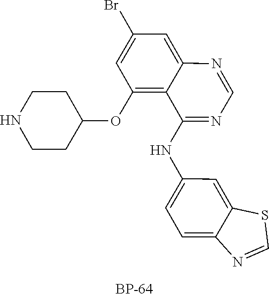 [SMILES: Brc1cc(OC2CCNCC2)c2c(Nc3ccc4ncsc4c3)ncnc2c1 ; "BP-64"] | | 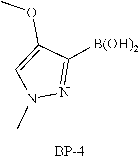 |
| 315 | 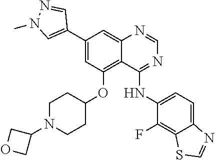 [SMILES: Cn1cc(-c2cc(OC3CCN(C4COC4)CC3)c3c(Nc4ccc5ncsc5c4F)ncnc3c2)cn1] | 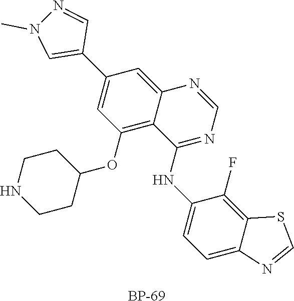 [SMILES: Cn1cc(-c2cc(OC3CCNCC3)c3c(Nc4ccc5ncsc5c4F)ncnc3c2)cn1 ; "BP-69"] | | 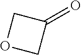 |
| 316 | 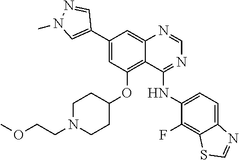 [SMILES: COCCN1CCC(Oc2cc(-c3cnn(C)c3)cc3ncnc(Nc4ccc5ncsc5c4F)c23)CC1] | 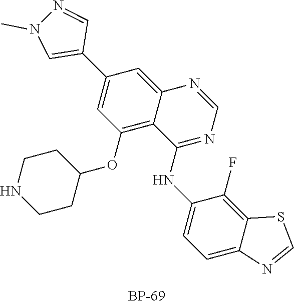 [SMILES: Cn1cc(-c2cc(OC3CCNCC3)c3c(Nc4ccc5ncsc5c4F)ncnc3c2)cn1 ; "BP-69"] | | 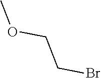 |
| 317 | 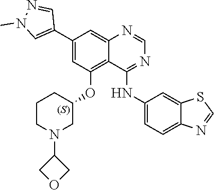 | 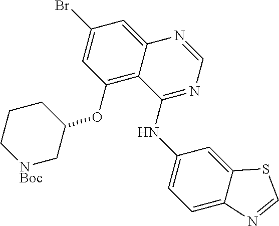 | | 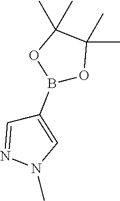 |
| 318 | 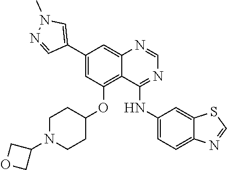 [SMILES: Cn1cc(-c2cc(OC3CCN(C4COC4)CC3)c3c(Nc4ccc5ncsc5c4)ncnc3c2)cn1] | 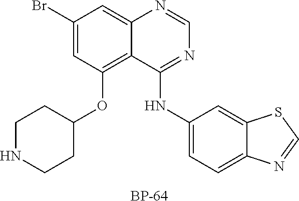 [SMILES: Brc1cc(OC2CCNCC2)c2c(Nc3ccc4ncsc4c3)ncnc2c1 ; "BP-64"] | | 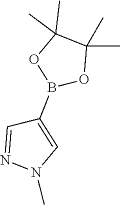 |
| 319 | 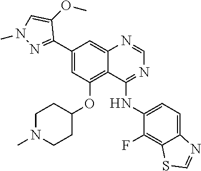 [SMILES: COc1cn(C)nc1-c1cc(OC2CCN(C)CC2)c2c(Nc3ccc4ncsc4c3F)ncnc2c1] | 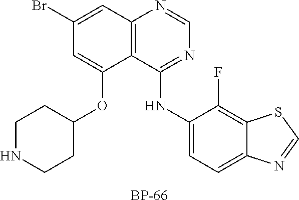 [SMILES: Fc1c(Nc2ncnc3cc(Br)cc(OC4CCNCC4)c23)ccc2ncsc12 ; "BP-66"] | | 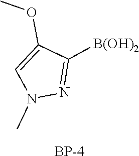 |
| 320 | 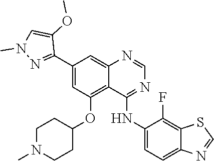 [SMILES: COc1cn(C)nc1-c1cc(OC2CCN(C)CC2)c2c(Nc3ccc4ncsc4c3F)ncnc2c1] | 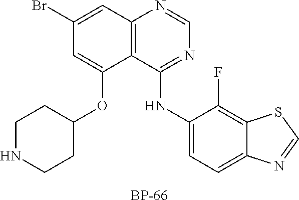 [SMILES: Fc1c(Nc2ncnc3cc(Br)cc(OC4CCNCC4)c23)ccc2ncsc12 ; "BP-66"] | | 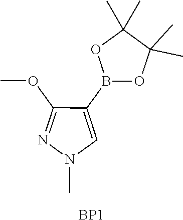 |
| 321 322 | 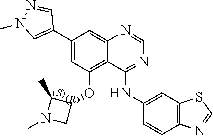 OR 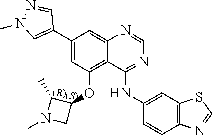 | 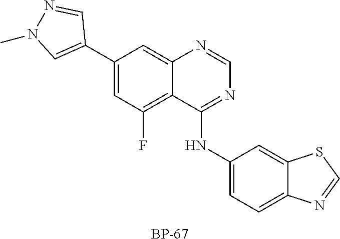 [SMILES: Cn1cc(-c2cc(F)c3c(Nc4ccc5ncsc5c4)ncnc3c2)cn1 ; "BP-67"] | | 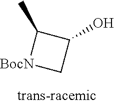 |
| 325 326 | 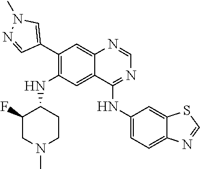 [SMILES: CN1CC[C@@H](Nc2cc3c(Nc4ccc5ncsc5c4)ncnc3cc2-c2cnn(C)c2)[C@H](F)C1] | 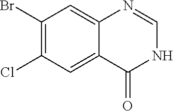 [SMILES: O=c1[nH]cnc2cc(Br)c(Cl)cc12] | | 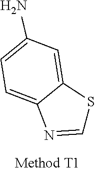 |
| | OR | | | |
| | 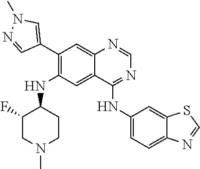 [SMILES: CN1CC[C@H](Nc2cc3c(Nc4ccc5ncsc5c4)ncnc3cc2-c2cnn(C)c2)[C@@H](F)C1] | | | |
| 327 328 | 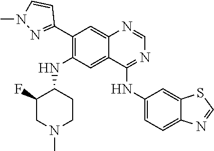 [SMILES: CN1CC[C@@H](Nc2cc3c(Nc4ccc5ncsc5c4)ncnc3cc2-c2ccn(C)n2)[C@H](F)C1] OR | 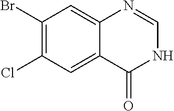 [SMILES: O=c1[nH]cnc2cc(Br)c(Cl)cc12] | | 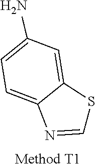 |
| | 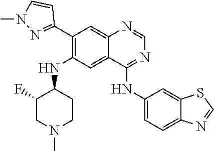 [SMILES: CN1CC[C@H](Nc2cc3c(Nc4ccc5ncsc5c4)ncnc3cc2-c2ccn(C)n2)[C@@H](F)C1] | | | |
| 329 | 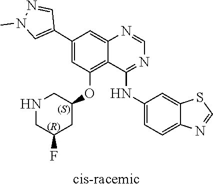 | 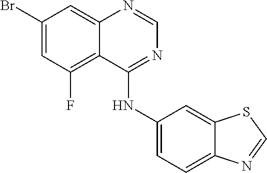 [SMILES: Fc1cc(Br)cc2ncnc(Nc3ccc4ncsc4c3)c12] | | 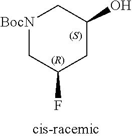 BP-40 |
| | | BP-67 | | |
| 331 | 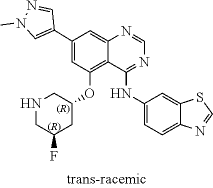 | 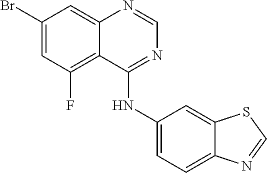 [SMILES: Fc1cc(Br)cc2ncnc(Nc3ccc4ncsc4c3)c12] | | 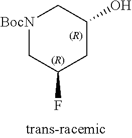 BP-41 |
| | | BP-59 | | |
| 332 333 | 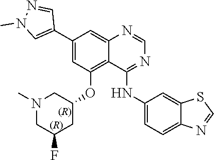 | Example 331 | | HCHO |
| | OR | | | |
| | 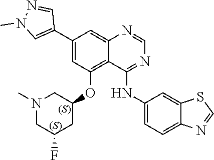 | | | |
| 334 335 | 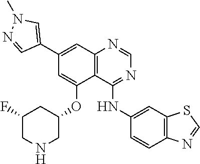 [SMILES: Cn1cc(-c2cc(O[C@@H]3CNC[C@H](F)C3)c3c(Nc4ccc5ncsc5c4)ncnc3c2)cn1] | Example 329 | | |
| | OR | | | |
| | 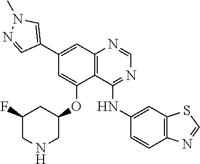 [SMILES: Cn1cc(-c2cc(O[C@H]3CNC[C@@H](F)C3)c3c(Nc4ccc5ncsc5c4)ncnc3c2)cn1] | | | |
| 336 337 | 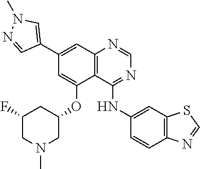 [SMILES: CN1C[C@H](F)C[C@H](Oc2cc(-c3cnn(C)c3)cc3ncnc(Nc4ccc5ncsc5c4)c23)C1] | Example 329 | | HCHO |
| | Or | | | |
| | 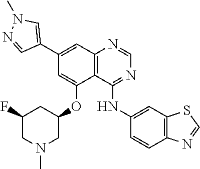 [SMILES: CN1C[C@@H](F)C[C@@H](Oc2cc(-c3cnn(C)c3)cc3ncnc(Nc4ccc5ncsc5c4)c23)C1] | | | |
| 339 | 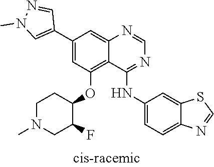 [SMILES: CN1CC[C@@H](Oc2cc(-c3cnn(C)c3)cc3ncnc(Nc4ccc5ncsc5c4)c23)[C@@H](F)C1] | 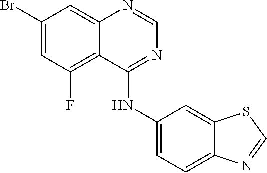 [SMILES: Fc1cc(Br)cc2ncnc(Nc3ccc4ncsc4c3)c12] | | 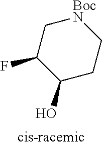 [SMILES: CC(C)(C)OC(=O)N1CC[C@@H](O)[C@@H](F)C1] |
| 340 341 | 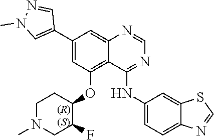 | Example 339 | | |
| 343 | 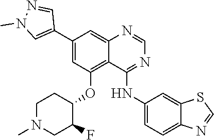 [SMILES: CN1CC[C@H](Oc2cc(-c3cnn(C)c3)cc3ncnc(Nc4ccc5ncsc5c4)c23)[C@@H](F)C1] | 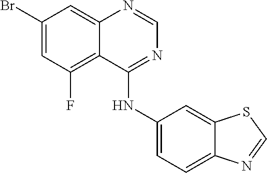 [SMILES: Fc1cc(Br)cc2ncnc(Nc3ccc4ncsc4c3)c12] | | 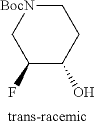 [SMILES: CC(C)(C)OC(=O)N1CC[C@H](O)[C@@H](F)C1] |
| | | BP-59 | | |
| 344 345 | 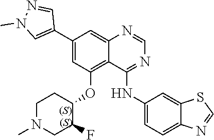 | Example 343 | | |
| | Or | | | |
| | 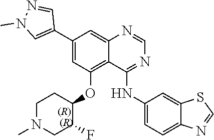 | | | |
| 346 347 | 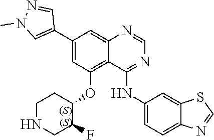 OR | 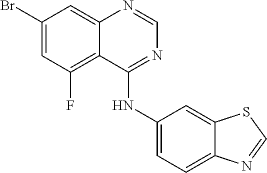 [SMILES: Fc1cc(Br)cc2ncnc(Nc3ccc4ncsc4c3)c12] | | |
| | 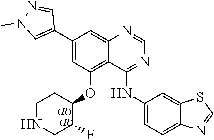 | BP-59 | | |
| 348 | 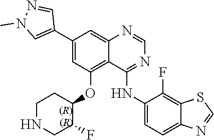 | 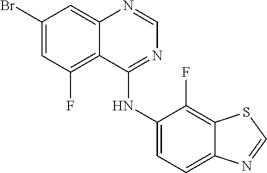 [SMILES: Fc1c(Nc2ncnc3cc(Br)cc(F)c23)ccc2ncsc12] | | 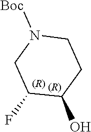 |
| | | BP-60 | | |
| 349 | 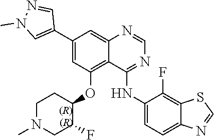 | Example 348 | | HCHO |
| 350 | 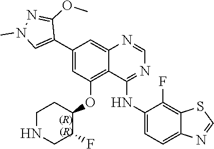 | 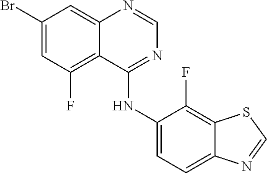 [SMILES: Fc1c(Nc2ncnc3cc(Br)cc(F)c23)ccc2ncsc12] | | 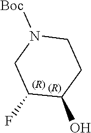 |
| | | BP-60 | | |
| 351 | 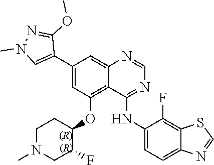 | Example 350 | | HCHO |
| 352 | 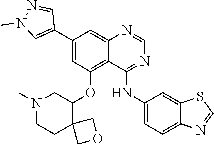 [SMILES: CN1CCC2(COC2)C(Oc2cc(-c3cnn(C)c3)cc3ncnc(Nc4ccc5ncsc5c4)c23)C1] | 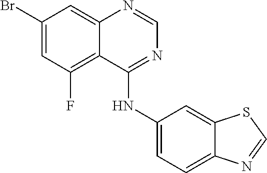 [SMILES: Fc1cc(Br)cc2ncnc(Nc3ccc4ncsc4c3)c12] | | 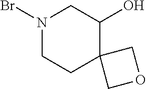 [SMILES: OC1CN(Br)CCC12COC2] |
| | | BP-59 | | |
| 353 354 | 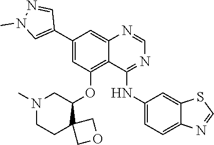 [SMILES: CN1CCC2(COC2)[C@H](Oc2cc(-c3cnn(C)c3)cc3ncnc(Nc4ccc5ncsc5c4)c23)C1] | Example 352 | | |
| | OR | | | |
| | 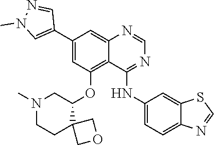 [SMILES: CN1CCC2(COC2)[C@@H](Oc2cc(-c3cnn(C)c3)cc3ncnc(Nc4ccc5ncsc5c4)c23)C1] | | | |
| 356 | 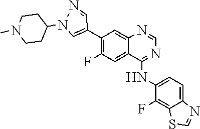 [SMILES: CN1CCC(n2cc(-c3cc4ncnc(Nc5ccc6ncsc6c5F)c4cc3F)cn2)CC1] | 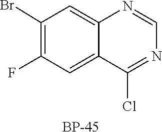 [SMILES: Fc1cc2c(Cl)ncnc2cc1Br ; "BP-45"] | | 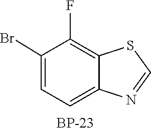 |
| 358 359 | 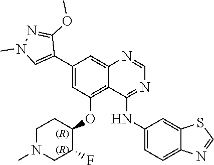 OR | 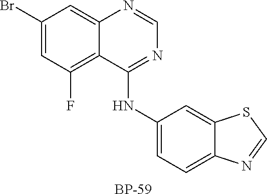 [SMILES: Fc1cc(Br)cc2ncnc(Nc3ccc4ncsc4c3)c12 ; "BP-59"] | | 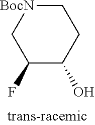 [SMILES: CC(C)(C)OC(=O)N1CC[C@H](O)[C@@H](F)C1] |
| | 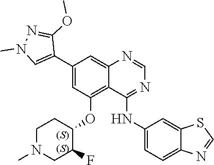 | | | |
| 360 361 | 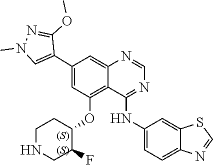 OR | 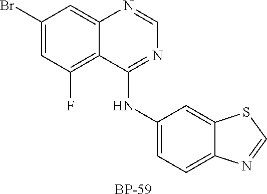 [SMILES: Fc1cc(Br)cc2ncnc(Nc3ccc4ncsc4c3)c12 ; "BP-59"] | | 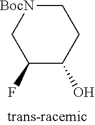 [SMILES: CC(C)(C)OC(=O)N1CC[C@H](O)[C@@H](F)C1] |
| | 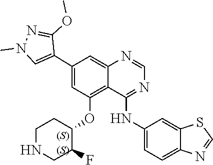 | | | |
| 362 | 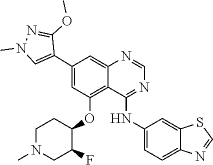 [SMILES: COc1nn(C)cc1-c1cc(O[C@@H]2CCN(C)C[C@@H]2F)c2c(Nc3ccc4ncsc4c3)ncnc2c1] | Example 363 | | |
| 363 | 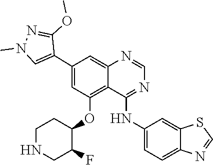 [SMILES: COc1nn(C)cc1-c1cc(O[C@@H]2CCNC[C@@H]2F)c2c(Nc3ccc4ncsc4c3)ncnc2c1] | 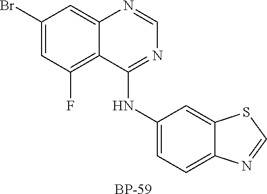 [SMILES: Fc1cc(Br)cc2ncnc(Nc3ccc4ncsc4c3)c12 ; "BP-59"] | | 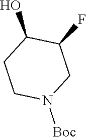 [SMILES: CC(C)(C)OC(=O)N1CC[C@@H](O)[C@@H](F)C1] |
| 364 | 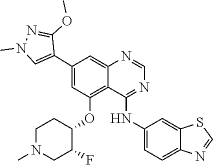 [SMILES: COc1nn(C)cc1-c1cc(O[C@H]2CCN(C)C[C@H]2F)c2c(Nc3ccc4ncsc4c3)ncnc2c1] | Example 365 | | |
| 365 | 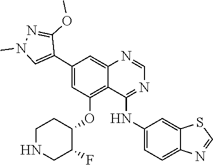 [SMILES: COc1nn(C)cc1-c1cc(O[C@H]2CCNC[C@H]2F)c2c(Nc3ccc4ncsc4c3)ncnc2c1] | 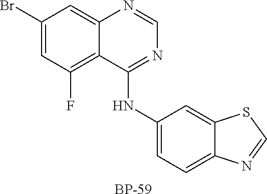 [SMILES: Fc1cc(Br)cc2ncnc(Nc3ccc4ncsc4c3)c12 ; "BP-59"] | | 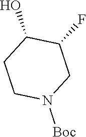 [SMILES: CC(C)(C)OC(=O)N1CC[C@H](O)[C@H](F)C1] |
| 366 | 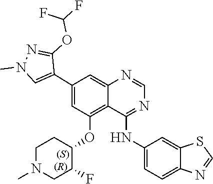 | 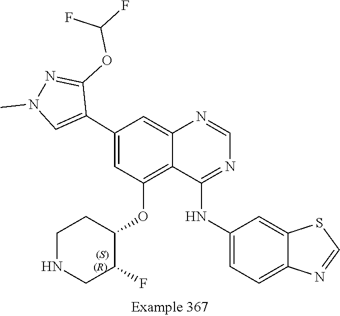 | | |
| 367 | 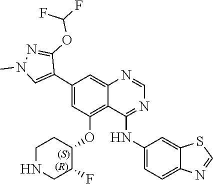 | 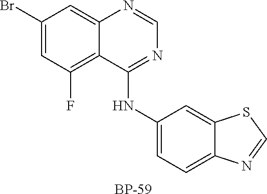 [SMILES: Fc1cc(Br)cc2ncnc(Nc3ccc4ncsc4c3)c12 ; "BP-59"] | | 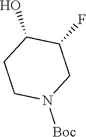 [SMILES: CC(C)(C)OC(=O)N1CC[C@H](O)[C@H](F)C1] |
| 368 369 | 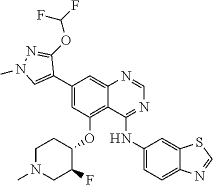 [SMILES: CN1CC[C@H](Oc2cc(-c3cn(C)nc3OC(F)F)cc3ncnc(Nc4ccc5ncsc5c4)c23)[C@@H](F)C1] | Example 370 | | HCHO |
| | OR | | | |
| | 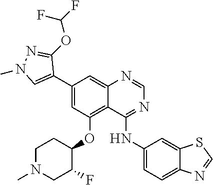 [SMILES: CN1CC[C@@H](Oc2cc(-c3cn(C)nc3OC(F)F)cc3ncnc(Nc4ccc5ncsc5c4)c23)[C@H](F)C1] | | | |
| 370 | 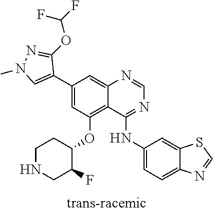 [SMILES: Cn1cc(-c2cc(O[C@H]3CCNC[C@@H]3F)c3c(Nc4ccc5ncsc5c4)ncnc3c2)c(OC(F)F)n1] | 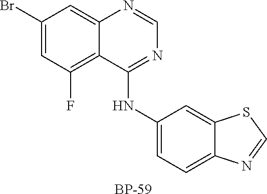 [SMILES: Fc1cc(Br)cc2ncnc(Nc3ccc4ncsc4c3)c12 ; "BP-59"] | | 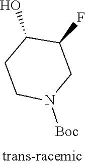 [SMILES: CC(C)(C)OC(=O)N1CC[C@H](O)[C@@H](F)C1] |
| 371 | 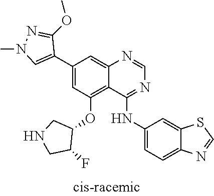 [SMILES: COc1nn(C)cc1-c1cc(O[C@H]2CNC[C@H]2F)c2c(Nc3ccc4ncsc4c3)ncnc2c1] | 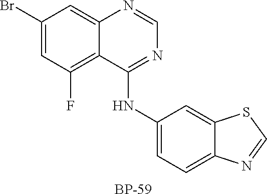 [SMILES: Fc1cc(Br)cc2ncnc(Nc3ccc4ncsc4c3)c12 ; "BP-59"] | | 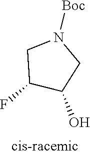 [SMILES: CC(C)(C)OC(=O)N1C[C@@H](F)[C@@H](O)C1] |
| 373 374 | 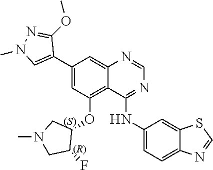 | Example 371 | | |
| | OR | | | |
| | 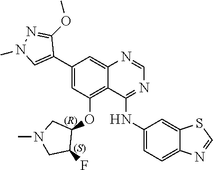 | | | |
| 375 | 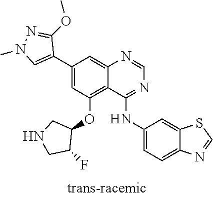 [SMILES: COc1nn(C)cc1-c1cc(O[C@@H]2CNC[C@H]2F)c2c(Nc3ccc4ncsc4c3)ncnc2c1] | 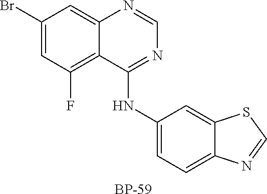 [SMILES: Fc1cc(Br)cc2ncnc(Nc3ccc4ncsc4c3)c12 ; "BP-59"] | | 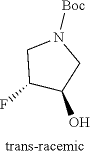 [SMILES: CC(C)(C)OC(=O)N1C[C@@H](O)[C@H](F)C1] |
| 376 | 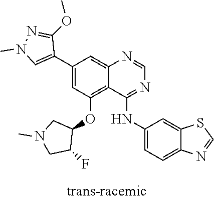 [SMILES: COc1nn(C)cc1-c1cc(O[C@@H]2CN(C)C[C@H]2F)c2c(Nc3ccc4ncsc4c3)ncnc2c1] | Example 375 | | |
| 377 378 | 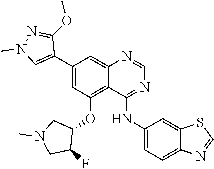 [SMILES: COc1nn(C)cc1-c1cc(O[C@H]2CN(C)C[C@@H]2F)c2c(Nc3ccc4ncsc4c3)ncnc2c1] | Example 376 | | |
| | OR | | | |
| | 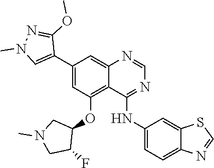 [SMILES: COc1nn(C)cc1-c1cc(O[C@@H]2CN(C)C[C@H]2F)c2c(Nc3ccc4ncsc4c3)ncnc2c1] | | | |
| 379 | 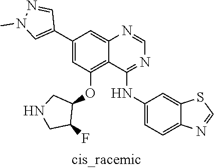 [SMILES: Cn1cc(-c2cc(O[C@@H]3CNC[C@@H]3F)c3c(Nc4ccc5ncsc5c4)ncnc3c2)cn1] | 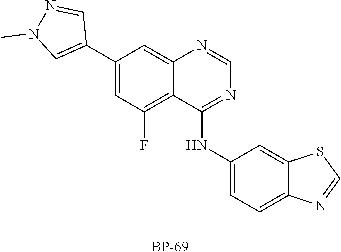 [SMILES: Cn1cc(-c2cc(F)c3c(Nc4ccc5ncsc5c4)ncnc3c2)cn1 ; "BP-69"] | | 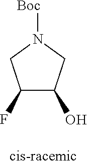 [SMILES: CC(C)(C)OC(=O)N1C[C@@H](O)[C@@H](F)C1] |
| 380 381 | 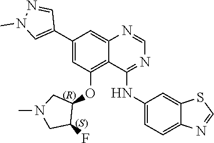 | Example 379 | | |
| | OR | | | |
| | 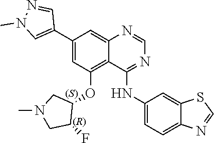 | | | |
| 382 | 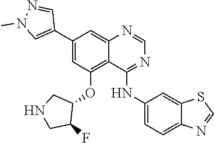 [SMILES: Cn1cc(-c2cc(O[C@H]3CNC[C@@H]3F)c3c(Nc4ccc5ncsc5c4)ncnc3c2)cn1] | 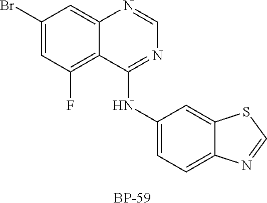 [SMILES: Fc1cc(Br)cc2ncnc(Nc3ccc4ncsc4c3)c12 ; "BP-59"] | | 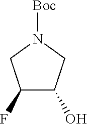 [SMILES: CC(C)(C)OC(=O)N1C[C@H](O)[C@@H](F)C1] |
| 383 | 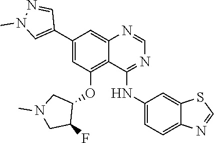 [SMILES: CN1C[C@H](Oc2cc(-c3cnn(C)c3)cc3ncnc(Nc4ccc5ncsc5c4)c23)[C@@H](F)C1] | Example 382 | | |
| 384 385 | 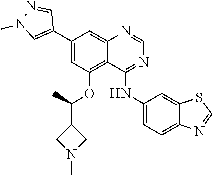 [SMILES: C[C@@H](Oc1cc(-c2cnn(C)c2)cc2ncnc(Nc3ccc4ncsc4c3)c12)C1CN(C)C1] OR | 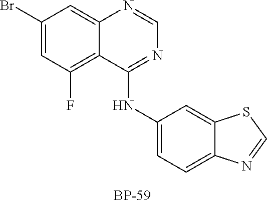 [SMILES: Fc1cc(Br)cc2ncnc(Nc3ccc4ncsc4c3)c12 ; "BP-59"] | | |
| | 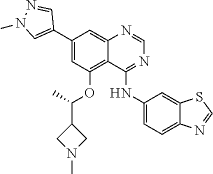 [SMILES: C[C@H](Oc1cc(-c2cnn(C)c2)cc2ncnc(Nc3ccc4ncsc4c3)c12)C1CN(C)C1] | | | |
| 386 | 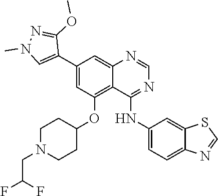 [SMILES: COc1nn(C)cc1-c1cc(OC2CCN(CC(F)F)CC2)c2c(Nc3ccc4ncsc4c3)ncnc2c1] | 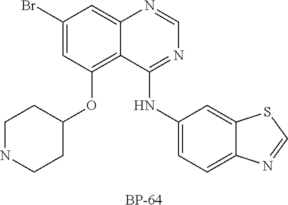 [SMILES: Brc1cc(OC2CCNCC2)c2c(Nc3ccc4ncsc4c3)ncnc2c1 ; "BP-64"] | | 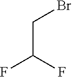 |
| 387 388 | 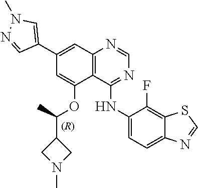 OR | 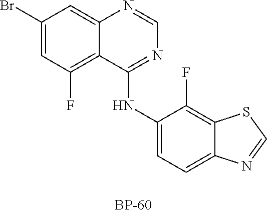 [SMILES: Fc1c(Nc2ncnc3cc(Br)cc(F)c23)ccc2ncsc12 ; "BP-60"] | | 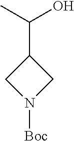 |
| | 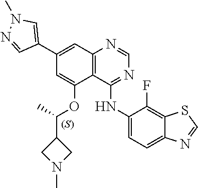 | | | |
| 389 390 | 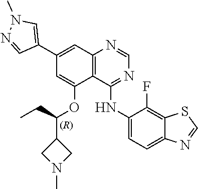 | 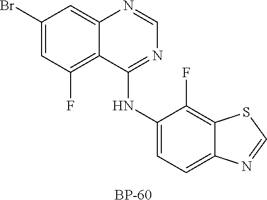 [SMILES: Fc1c(Nc2ncnc3cc(Br)cc(F)c23)ccc2ncsc12 ; "BP-60"] | | 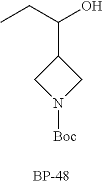 |
| | OR | | | |
| | 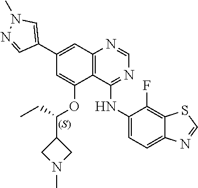 | | | |
| 393 | 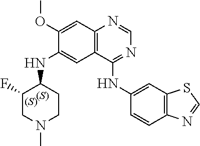 | 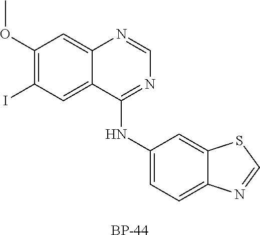 [SMILES: COc1cc2ncnc(Nc3ccc4ncsc4c3)c2cc1I ; "BP-44"] | | 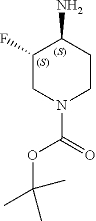 |
| 394 | 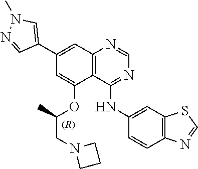 | 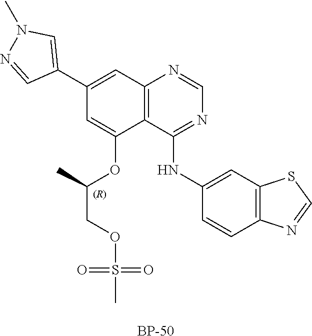 | | 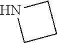 |
| 395 | 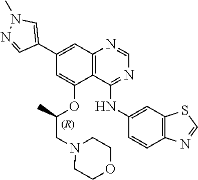 | 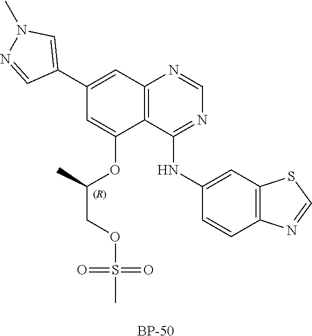 | | 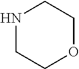 |
| 396 | 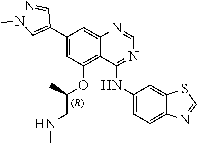 | 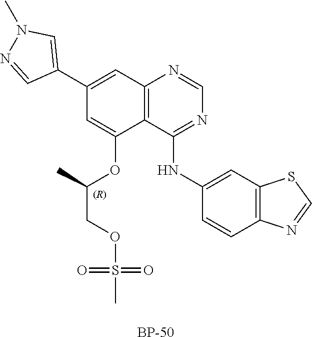 | | 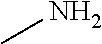 |
| 397 | 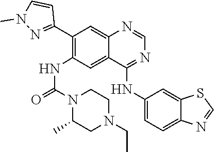 [SMILES: CCN1CCN(C(=O)Nc2cc3c(Nc4ccc5ncsc5c4)ncnc3cc2-c2ccn(C)n2)[C@@H](C)C1] | 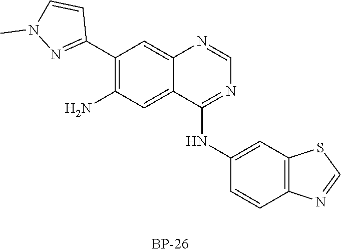 [SMILES: Cn1ccc(-c2cc3ncnc(Nc4ccc5ncsc5c4)c3cc2N)n1 ; "BP-26"] | | 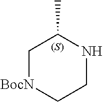 |
| 398 | 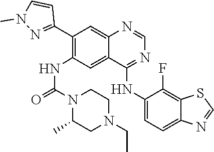 [SMILES: CCN1CCN(C(=O)Nc2cc3c(Nc4ccc5ncsc5c4F)ncnc3cc2-c2ccn(C)n2)[C@@H](C)C1] | 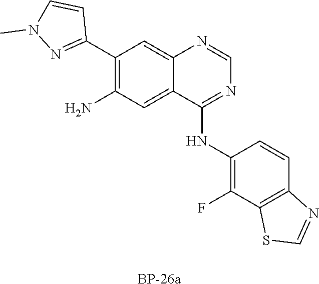 [SMILES: Cn1ccc(-c2cc3ncnc(Nc4ccc5ncsc5c4F)c3cc2N)n1 ; "BP-26a"] | | 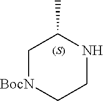 |
| 399 | 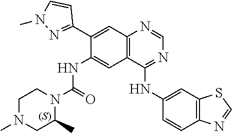 | 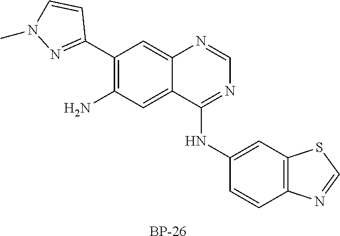 [SMILES: Cn1ccc(-c2cc3ncnc(Nc4ccc5ncsc5c4)c3cc2N)n1 ; "BP-26"] | | 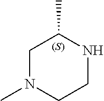 |
| 400 | 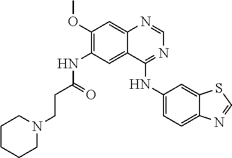 [SMILES: COc1cc2ncnc(Nc3ccc4ncsc4c3)c2cc1NC(=O)CCN1CCCCC1] | 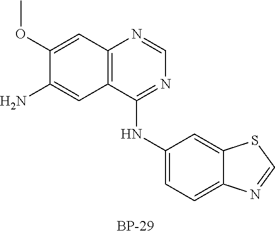 [SMILES: COc1cc2ncnc(Nc3ccc4ncsc4c3)c2cc1N ; "BP-29"] | | 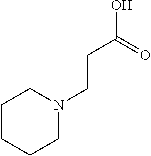 [SMILES: O=C(O)CCN1CCCCC1] |
| 401 | 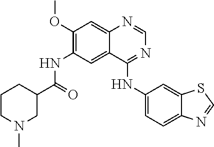 [SMILES: COc1cc2ncnc(Nc3ccc4ncsc4c3)c2cc1NC(=O)C1CCCN(C)C1] | 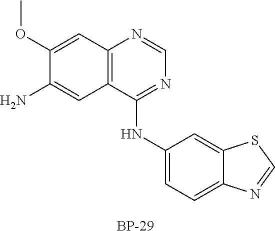 [SMILES: COc1cc2ncnc(Nc3ccc4ncsc4c3)c2cc1N ; "BP-29"] | | 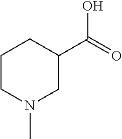 |
| 402 | 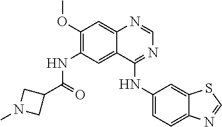 [SMILES: COc1cc2ncnc(Nc3ccc4ncsc4c3)c2cc1NC(=O)C1CN(C)C1] | 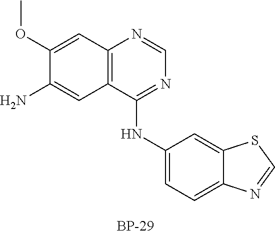 [SMILES: COc1cc2ncnc(Nc3ccc4ncsc4c3)c2cc1N ; "BP-29"] | | 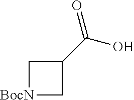 [SMILES: CC(C)(C)OC(=O)N1CC(C(=O)O)C1] |
| 405 | 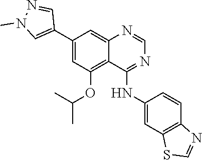 [SMILES: CC(C)Oc1cc(-c2cnn(C)c2)cc2ncnc(Nc3ccc4ncsc4c3)c12] | 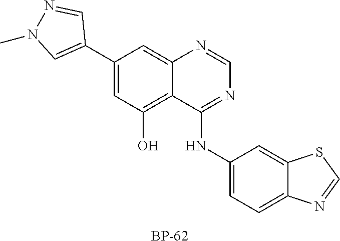 [SMILES: Cn1cc(-c2cc(O)c3c(Nc4ccc5ncsc5c4)ncnc3c2)cn1 ; "BP-62"] | | 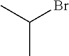 |
| 406 | 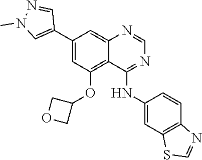 [SMILES: Cn1cc(-c2cc(OC3COC3)c3c(Nc4ccc5ncsc5c4)ncnc3c2)cn1] | 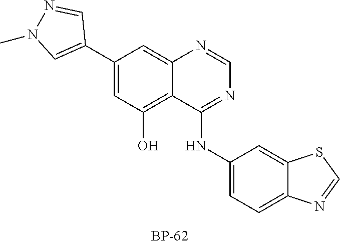 [SMILES: Cn1cc(-c2cc(O)c3c(Nc4ccc5ncsc5c4)ncnc3c2)cn1 ; "BP-62"] | | 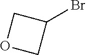 |
| 407 | 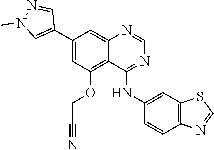 [SMILES: Cn1cc(-c2cc(OCC#N)c3c(Nc4ccc5ncsc5c4)ncnc3c2)cn1] | 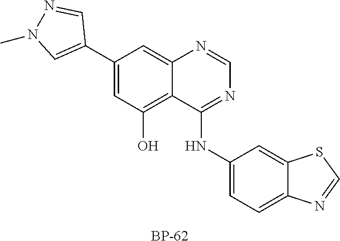 [SMILES: Cn1cc(-c2cc(O)c3c(Nc4ccc5ncsc5c4)ncnc3c2)cn1 ; "BP-62"] | | 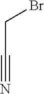 |
| 409 | 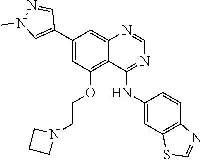 [SMILES: Cn1cc(-c2cc(OCCN3CCC3)c3c(Nc4ccc5ncsc5c4)ncnc3c2)cn1] | 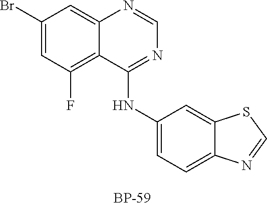 [SMILES: Fc1cc(Br)cc2ncnc(Nc3ccc4ncsc4c3)c12 ; "BP-59"] | | 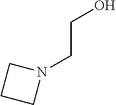 |
| 410 | 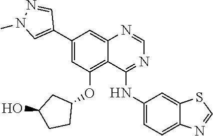 [SMILES: Cn1cc(-c2cc(O[C@@H]3CC[C@@H](O)C3)c3c(Nc4ccc5ncsc5c4)ncnc3c2)cn1] | 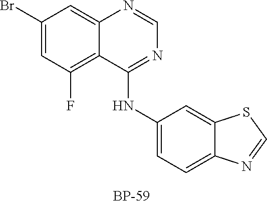 [SMILES: Fc1cc(Br)cc2ncnc(Nc3ccc4ncsc4c3)c12 ; "BP-59"] | | 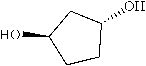 |
| 411 | 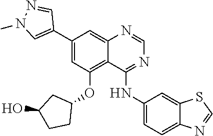 [SMILES: Cn1cc(-c2cc(O[C@@H]3CC[C@@H](O)C3)c3c(Nc4ccc5ncsc5c4)ncnc3c2)cn1] | 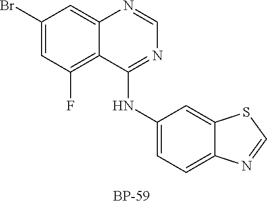 [SMILES: Fc1cc(Br)cc2ncnc(Nc3ccc4ncsc4c3)c12 ; "BP-59"] | | 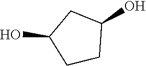 |
| 412 | 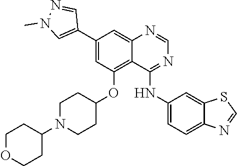 [SMILES: Cn1cc(-c2cc(OC3CCN(C4CCOCC4)CC3)c3c(Nc4ccc5ncsc5c4)ncnc3c2)cn1] | 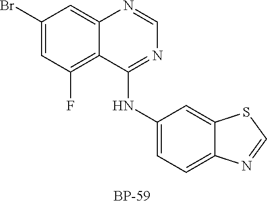 [SMILES: Fc1cc(Br)cc2ncnc(Nc3ccc4ncsc4c3)c12 ; "BP-59"] | | 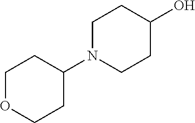 [SMILES: OC1CCN(C2CCOCC2)CC1] |
| 413 | 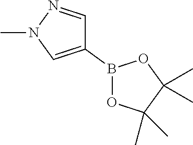 | 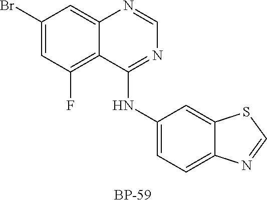 [SMILES: Fc1cc(Br)cc2ncnc(Nc3ccc4ncsc4c3)c12 ; "BP-59"] | | 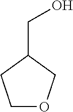 |
| 414 | 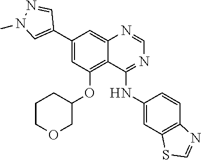 [SMILES: Cn1cc(-c2cc(OC3CCCOC3)c3c(Nc4ccc5ncsc5c4)ncnc3c2)cn1] | 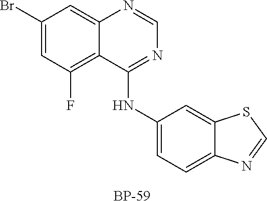 [SMILES: Fc1cc(Br)cc2ncnc(Nc3ccc4ncsc4c3)c12 ; "BP-59"] | | 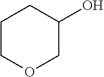 |
| 415 | 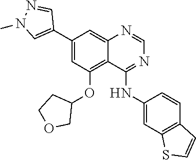 [SMILES: Cn1cc(-c2cc(OC3CCOC3)c3c(Nc4ccc5ccsc5c4)ncnc3c2)cn1] | 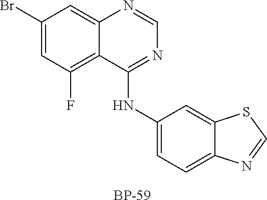 [SMILES: Fc1cc(Br)cc2ncnc(Nc3ccc4ncsc4c3)c12 ; "BP-59"] | | 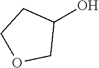 |
| | | | | Methods |
| | Example # | Reactant 2 | | (In order used) |
| | 1 | 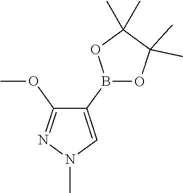 | | A1, B1 |
| | | BP-1 | | |
| | 2 | 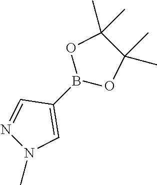 | | A1, B1 |
| | 3 | 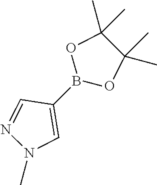 | | A2 (methanol as solvent), B1 |
| | 4 | 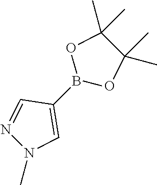 | | A3, B1 |
| | 5 | 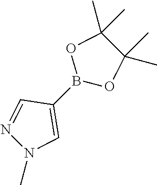 | | A3, B2 |
| | 6 7 | 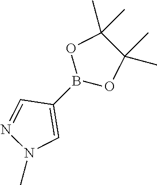 | | A3, B1 Chiral Separation R or S |
| | 8 | 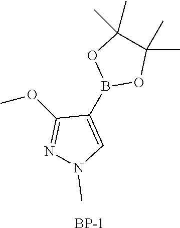 | | A1, B2 |
| | 9 | 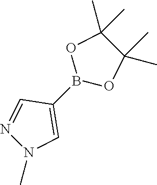 | | A3, B1 isomers separated by prep-TLC |
| | 10 | | | |
| | 11 | 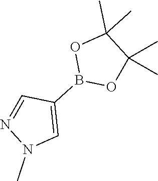 | | A3, B2 |
| | 12 | 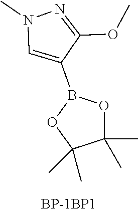 | | A1, B1 |
| | 13 | 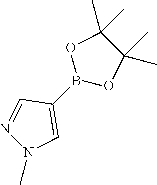 | | A3, B2 |
| | 14 | 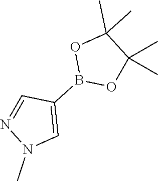 | | A1, B2 |
| | 15 | | | B1 |
| | 16 | 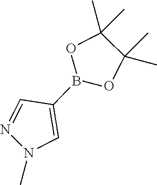 | | A3, B1 |
| | 17 | 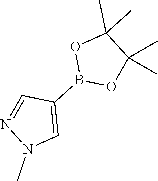 | | A1, B1 |
| | 18 | 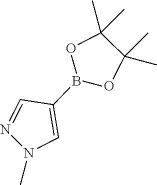 | | A1, B1 |
| | 19 | 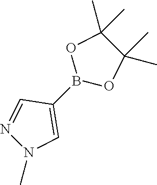 | | A3, B1, D1 |
| | 20 | 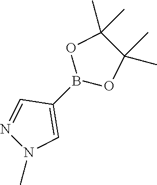 | | A3, B1, D1 |
| | 21 | — | | A1 |
| | 22 | 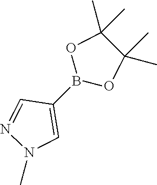 | | A3, B1 |
| | 23 24 | 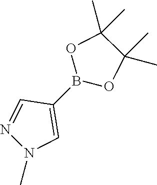 | | A1, B1, Chiral Separation |
| | 25 | 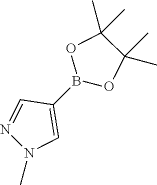 | | A3, B1 isomers separated by prep-TLC |
| | 26 | | | |
| | 27 | — | | A1 |
| | 28 | — | | A1 |
| | 29 | — | | A1, D2 |
| | 30 | — | | A1 |
| | 31 | — | | A1 |
| | 32 | — | | A1 |
| | 33 | — | | A1 |
| | 34 | — | | A1 |
| | 35 | — | | A1 |
| | 36 | — | | A1 |
| | 37 | — | | A1 |
| | 38 | — | | A1 |
| | 41 42 | — | | A1 Chiral Separation |
| | 43 44 | — | | A1 Chiral Separation |
| | 45 46 | — | | A1 Chiral Separation |
| | 47 48 | — | | A1 Chiral Separation |
| | 49 | 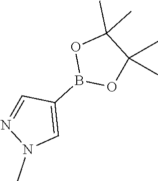 | | A1, B1 |
| | 50 | 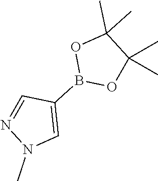 | | A1, B1 |
| | 51 | 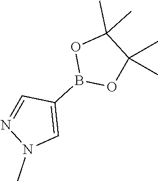 | | A1, B1 |
| | 52 | | | Chiral |
| | 53 | | | Separation |
| | 54 | 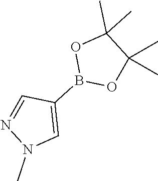 | | A1, B1 |
| | 55 | | | Chiral |
| | 56 | | | Separation |
| | 57 | 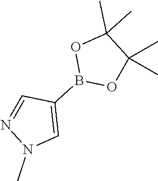 | | A1, B1 |
| | 58 | 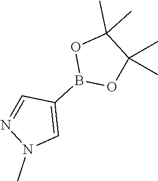 | | A1, B1 |
| | 59 | | | Chiral |
| | 60 | | | Separation |
| | 61 | 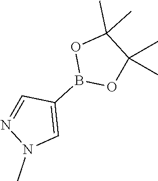 | | A1, B1 |
| | 62 | 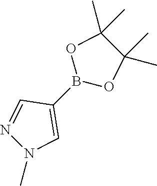 | | A1, B1 |
| | 63 | — | | Chiral |
| | 64 | | | Separation |
| | 65 | 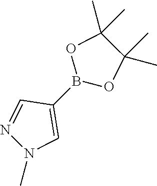 | | A1, B1 |
| | 66 | 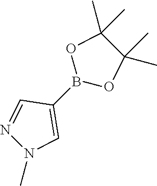 | | A1, B1 |
| | 67 | 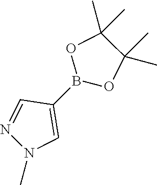 | | A1, B1 |
| | 68 | 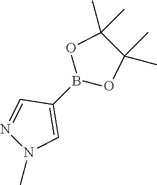 | | A1, B1 |
| | 69 | 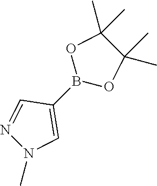 | | A1, B1 |
| | 70 71 | 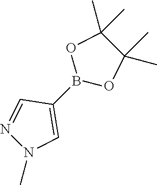 | | A3, B1 Chiral Separation |
| | 72 73 | 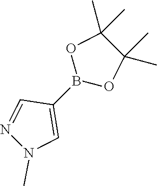 | | A1, B1 Chiral Separation |
| | 74 75 | 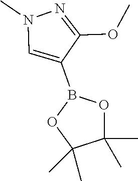 | | A1, B1 Chiral Separation |
| | | BP-1 | | |
| | 76 | 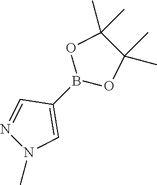 | | A2 (+ 1 eq NaI), B1 |
| | 77 | 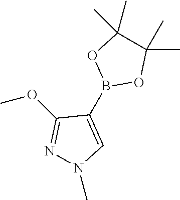 | | A2 (+ 1 eq NaI), B1 |
| | | BP-1 | | |
| | 78 79 | | | B1 Chiral Separation |
| | 80 81 | | | B1 Chiral Separation |
| | 82 83 | | | B3 Chiral Separation |
| | 84 85 | | | B3 Chiral Separation |
| | 86 87 | 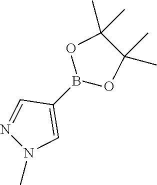 | | A3, B1 Chiral Separation |
| | 88 89 | 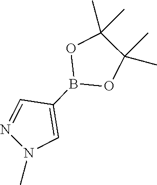 | | A1, B1 Chiral Separation |
| | 90 | 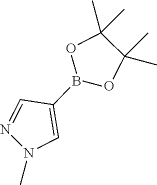 | | A1, B1 |
| | 91 | 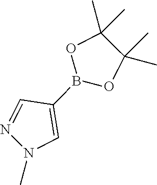 | | A3, B1 |
| | 92 93 | 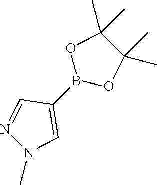 | | A1, B1 Chiral Separation |
| | 94 95 | | | B5a, Chiral Separation |
| | 96 97 | | | B5c, Chiral Separation |
| | 98 99 | | | B5a, Chiral Separation |
| | 100 101 | | | 5Bb, D2 Chiral Separation |
| | 102 103 | HCHO | | 5Bb, D2, C1, Chiral Separation |
| | 104 105 | | | B5b, D2 Chiral Separation |
| | 106 107 | HCHO | | B5b, D2, C1 Chiral Separation |
| | 108 109 | 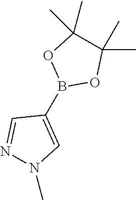 | | A1, B1 Chiral Separation |
| | 110 111 | 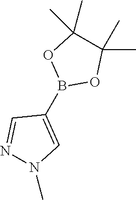 | | A3, B1 |
| | 112 | 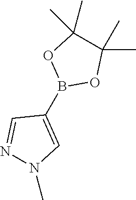 | | A3, B1 |
| | 113 | 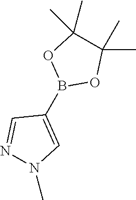 | | A3, B1 |
| | 114 | 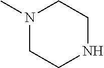 | | A3, B5b |
| | 115 | 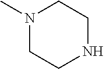 | | A3, B5b |
| | 116 117 | 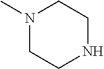 | | A3, B5c, Chiral Separation |
| | 118 119 | 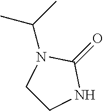 | | A3, B5d, Chiral Separation |
| | 120 | | | B1, |
| | 121 | | | Chiral |
| | | | | Separation |
| | 122 | | | B1 |
| | 123 | | | Chiral |
| | | | | Separation |
| | 124 | | | B3 |
| | 125 | | | B3 |
| | | | | (IPA as solvent) |
| | 126 | | | B3 |
| | 127 128 | 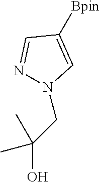 | | A3, B1, Chiral Spearation |
| | 129 | | | B5b |
| | 130 | | | B3 |
| | 131 | 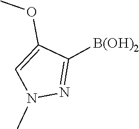 | | A1, B1 |
| | 132 | | | B1b |
| | 133 | 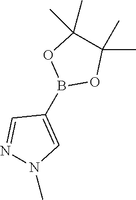 | | A3, B1 |
| | 134 | | | B1 |
| | 135 | | | B5f |
| | 136 | | | Prep-HPLC |
| | | | | separation |
| | | | | of isomers |
| | 139 | 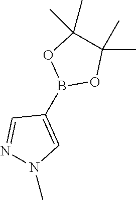 | | A3, B1 |
| | 140 | 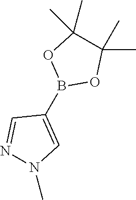 | | A1, B1 |
| | 141 | 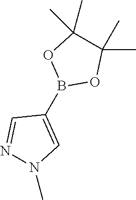 | | A3, B1 |
| | 142 143 | 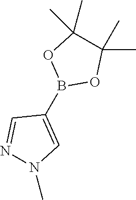 | | A1, B1 |
| | 144 145 | 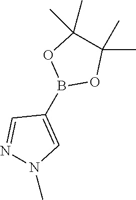 | | A3, B1 |
| | 146 | MeOH | | A1, B3 |
| | 147 148 | 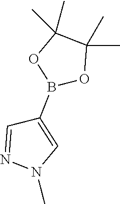 | | A3, B5e Chiral Separation |
| | 149 150 | 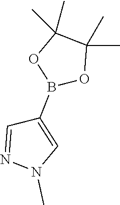 | | A3, B5c Chiral Separation |
| | 151 152 | 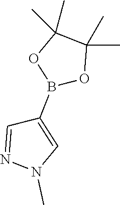 | | A3, B5e Chiral Separation |
| | 153 154 | 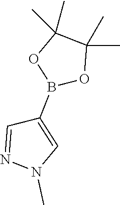 | | A3, B5e Chiral Separation |
| | 155 156 | — | | B5c, Chiral Separation |
| | 157 158 | | | B5d, Chiral Separation |
| | 159 160 | | | B5e, Chiral Separation |
| | 161 162 | | | A1, D2, T4 Chiral Separation |
| | 163 | | | B1b |
| | 164 | | | B1, D2 |
| | 165 | | | B1, D2, T4 |
| | 166 167 | | | Chiral Separation |
| | 168 | | | B1, D2 |
| | 169 | | | T4 |
| | 170 171 | | | B2 Chiral Separation |
| | 172 173 | 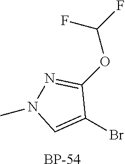 | | B7, B1 Chiral Separation |
| | 174 175 | 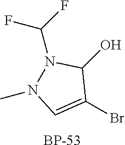 | | B7, B1 Chiral Separation |
| | 176 | | | B5b |
| | 177 | | | B5a |
| | 178 179 | 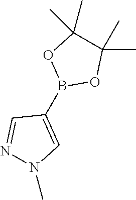 | | A3, B1e, Chiral Separation |
| | 180 | | | B5b |
| | 181 | | | B5b |
| | 182 | | | B5b |
| | 183 | | | B5b |
| | 184 | 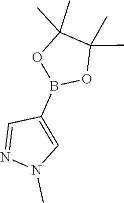 | | A3b, B5e |
| | 185 | 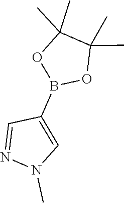 | | A3b, B5e |
| | 186 | 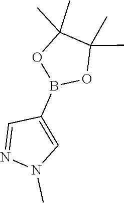 | | A3b, B5e |
| | 187 | 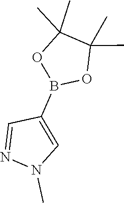 | | A3b, B5e |
| | 188 | 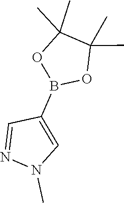 | | A3b, B5e |
| | 189 | 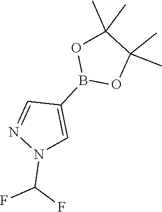 [SMILES: CC1(C)OB(c2cnn(C(F)F)c2)OC1(C)C] | | A1, B5e (MW@ 150° C 2 hr.) |
| | 190 | | | B5e |
| | 191 | | | B5e |
| | 192 | 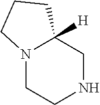 | | A3, B5c |
| | 193 | | | B5c |
| | 194 | | | B5d |
| | 195 | 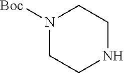 [SMILES: CC(C)(C)OC(=O)N1CCNCC1] | | A3, B5c, D2 |
| | 196 | 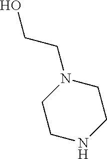 | | A3, B5c |
| | 197 | | | B5c |
| | 198 | | | B5c |
| | 199 | | | B5d |
| | 200 | | | B5i |
| | 201 | 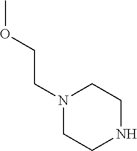 | | A3, B5c |
| | 202 | | | B5h |
| | 203 | | | B5h |
| | 204 | | | B5c |
| | 205 206 | 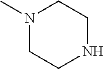 | | A3, B5c |
| | 207 208 | 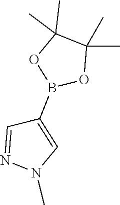 | | A3, B1, D2 Chiral Separation |
| | 209 210 | 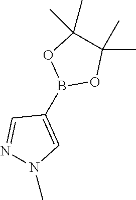 | | |
| | 211 212 | 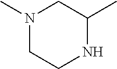 | | A3, B5c, Chiral Separation |
| | # | Reactant 2 | Reactant 3 | Methods in order of use |
| | 226 | | — | B1b, D2, T4c |
| | 227 | | — | B1b, D2, T4c |
| | 240 | 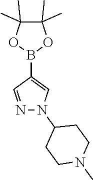 | | T1, B1b |
| | 241 | 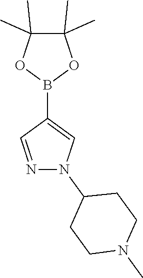 | | T1, B1b |
| | 242 | | | B2, D2, T4 |
| | 243 | 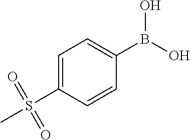 | | A1, B5e |
| | 245 | 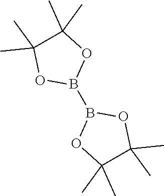 | 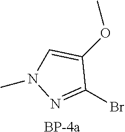 | A3, B7, B1 |
| | 246 | 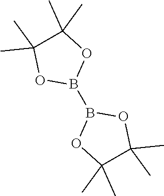 | 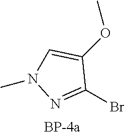 | A3, B7, B1 |
| | 247 | 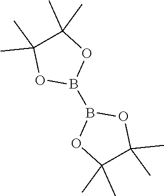 | 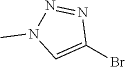 | A3, B7, B1 |
| | 248 | 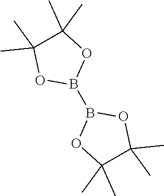 | 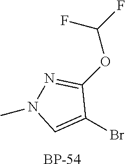 | A3, B7, B1 |
| | 249 | 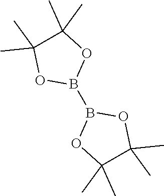 | 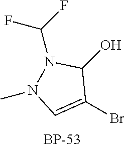 | A3, B7, B1 |
| | 250 | 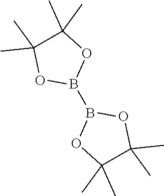 | 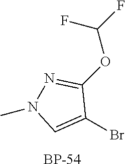 | A1, B7 (Pd(PPh₃)₄), B1 |
| | # | Reactant 2 | | reactant 3 |
| | 251 | — | | — |
| | 252 | — | | — |
| | 253 | — | | — |
| | 254 | 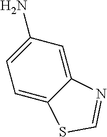 | | 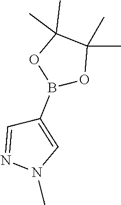 |
| | 255 | | | — |
| | 256 | 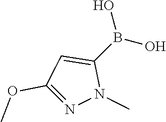 | | 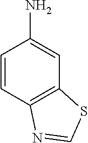 |
| | 257 | — | | — |
| | 258 | — | | — |
| | 259 | 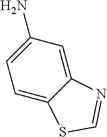 | | 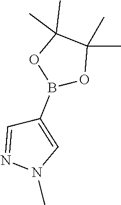 |
| | 260 | 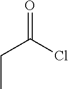 | | — |
| | 261 | 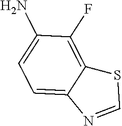 [SMILES: Nc1ccc2ncsc2c1F] | | 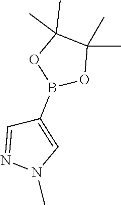 |
| | 262 | 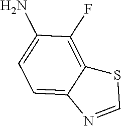 [SMILES: Nc1ccc2ncsc2c1F] | | 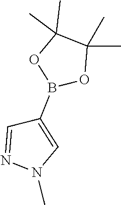 |
| | 263 | 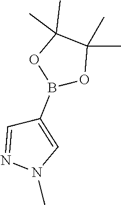 | | — |
| | 264 | 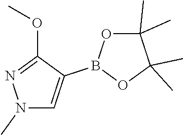 | | — |
| | 265 | 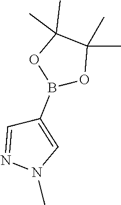 | | |
| | # | Reactant 2 | Reactant 3 | Methods in order of use |
| | 270 | 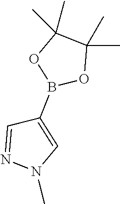 | 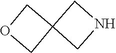 | A3, B1, E1, E2 |
| | 271 | 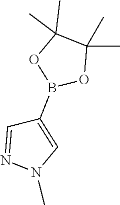 | 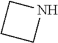 | A3, B1, E1, E2 |
| | 272 | 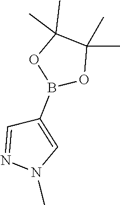 | 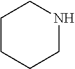 | A3, B1, E1, E2 |
| | 273 | 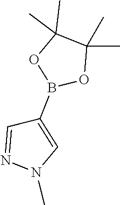 | 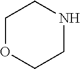 | A3, B1, E1, E2 |
| | 274 | 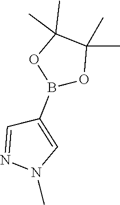 | 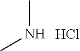 | A3, B1, E1, E2 |
| | 275 | 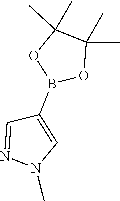 | 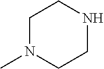 | A3, B1, E1, E2 |
| | 276 | 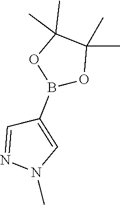 | 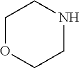 | A3, B1, E1, E2 |
| | 277 | 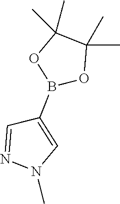 | 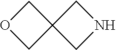 | A3, B1, E1, E2 |
| | 278 | 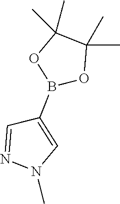 | | A3, B1 (ester hydrolyzed in this step), E2 |
| | 279 | | | Chiral Separation |
| | 280 | | | |
| | 281 | 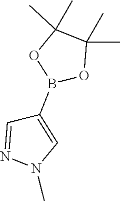 | 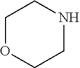 | A3, B1, E1, E2 |
| | 282 | | | Chiral Separation |
| | 283 | | | |
| | 284 | 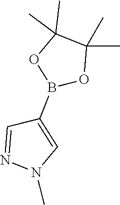 | 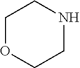 | A3, B1 (ester hydrolized in this step), E2 |
| | 285 | 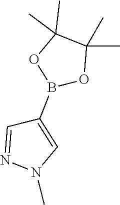 | 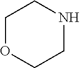 | A3, B1 (ester hydrolized in this step), E2 |
| | 286 | 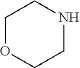 | — | A5, E1, E2 |
| | 287 288 | 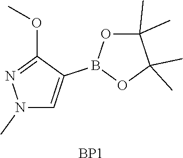 | 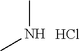 | A3, B1, E1, E2, Chiral Separation |
| | 289 290 | 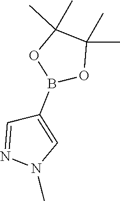 | | A3, E5, B1, Chiral Separation |
| | 291 | | | B1, E5, |
| | 292 | | | Chiral Separation |
| | 293 294 | 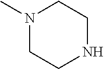 | | A1, E5, B5b, Chiral Separation |
| | 295 296 | 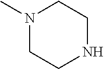 | | A1, E5, B5b, Chiral Separation |
| | 297 | | | B1, E5, |
| | 298 | | | Chiral Separation |
| | 299 | HCHO | | B5b, D2, C1, |
| | 300 | | | Chiral Separation |
| | 301 | HCHO | | B5g, D2, C1 |
| | # | Reactant 2 | | Methods (In reaction order) |
| | 305 | 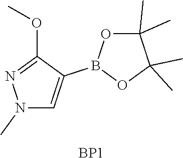 | | A4, B1 |
| | 306 | 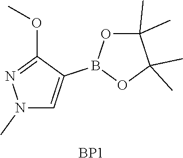 | | D2, C1, B1 |
| | 307 | 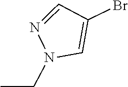 | | D2, C1, B1 |
| | 308 | 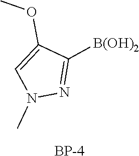 | | A4, B1 |
| | 309 | 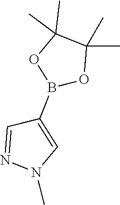 | | A4, B1 |
| | 310 | 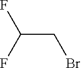 | | B1, D2, A4, |
| | 311 | | | A4 |
| | 312 | | | A4, D2 |
| | 313 | | | A4 |
| | 314 | | | B1, T4 |
| | 315 | | | T4b |
| | 316 | | | A4 |
| | 317 | 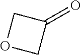 | | B1, D2, C1 |
| | 318 | 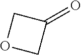 | | B1, C1 |
| | 319 | | | T4, B1 |
| | 320 | | | T4, B1 |
| | 321 | | | A1, D2, |
| | 322 | | | T4 Chiral Separation |
| | # | Reagent 2 | Reagent 3 | Methods (In reaction order) |
| | 325 326 | 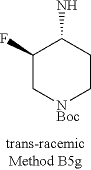 [SMILES: CC(C)(C)OC(=O)N1CC[C@@H](N)[C@H](F)C1] | 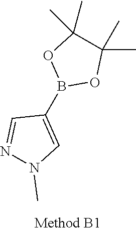 | C2, T1, B5g, B1, D2, T4, Chiral Separation |
| | 327 328 | 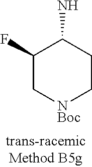 [SMILES: CC(C)(C)OC(=O)N1CC[C@@H](N)[C@H](F)C1] | 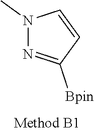 | C2, T1, B5g, B1, D2, T4, Chiral Separation |
| | 329 | 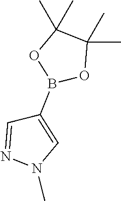 | | A1, B1, D2 |
| | 331 | 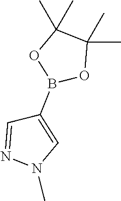 | | A3, B1, D2 |
| | 332 | | | C1, Chiral Separation |
| | 333 | | | |
| | 334 | | | Chiral Separation |
| | 335 | | | |
| | 336 | | | C1, Chiral Separation |
| | 337 | | | |
| | 339 | 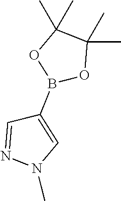 | | A1, B1b, D2, T4 |
| | 340 | | | Chiral Separation |
| | 341 | | | |
| | 343 | 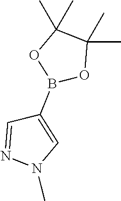 | | A1, B1, D2, T4 |
| | 344 | | | Chiral Separation |
| | 345 | | | |
| | 346 347 | 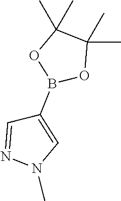 | | A1, B1, D2, Chiral Separation |
| | 348 | 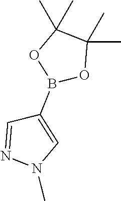 | | A1, B1, D2 |
| | 349 | | | C1 |
| | 350 | 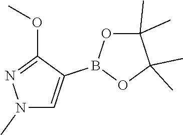 | | A1, B1, D2 |
| | 351 | | | C1 |
| | 352 | 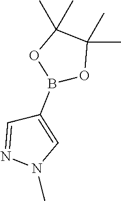 | HCHO | A3, B1, D2, C1 |
| | 353 | | | Chiral Separation |
| | 354 | | | |
| | 356 | 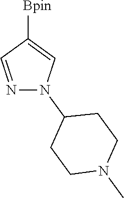 | | T1d, B1 |
| | 358 359 | 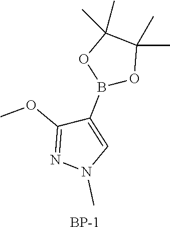 | | A1, B1, D2, T4, Chiral Separation |
| | 360 361 | 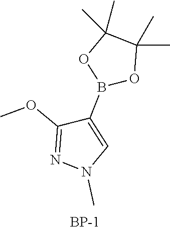 | | A1, B1, D2 Chiral Separation |
| | 362 | | | T4 |
| | 363 | 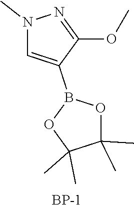 | | A1, B1, D2 |
| | 364 | | | T4 |
| | 365 | 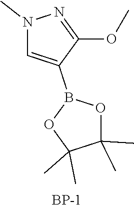 | | A1, B1, D2 |
| | 366 | | | T4 |
| | 367 | 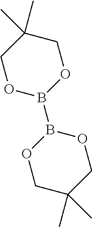 [SMILES: CC1(C)COB(B2OCC(C)(C)CO2)OC1] | 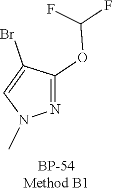 | A1, B6, B1, D2 |
| | 368 | | | C1, Chiral Separation |
| | 369 | | | |
| | 370 | 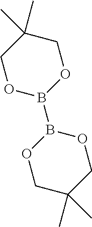 [SMILES: CC1(C)COB(B2OCC(C)(C)CO2)OC1] | 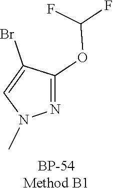 | A1, B6, B1, D2 |
| | 371 | 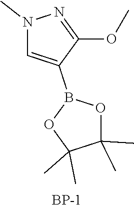 | | A1, B1, D2 |
| | 373 | | | T4, Chiral Separation |
| | 374 | | | |
| | 375 | | | A1, B1, D2 |
| | 376 | | | T4 |
| | 377 | | | Chiral Separation |
| | 378 | | | |
| | 379 | | | A1, D2 |
| | 380 | | | T4, Chiral Separation |
| | 381 | | | |
| | 382 | 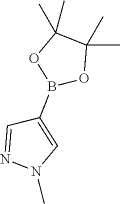 | | A1, B1, D2 |
| | 383 | | | T4 |
| | 384 385 | 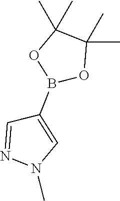 | | A1, B1, D2, T4, Chiral Separation |
| | 386 | 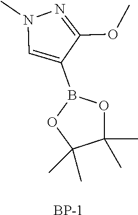 | | A4, B1 |
| | 387 388 | 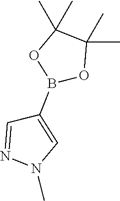 | HCHO | A1, B1, D2, C1, Chiral Separation |
| | 389 390 | 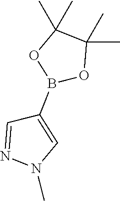 | | A1, B1, D2, T4, Chiral Separation |
| | 393 | HCHO | | B5g, D2, C1 |
| | 394 | | | A4b |
| | 395 | | | A4b |
| | 396 | | | A4b |
| | 397 | 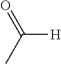 | | T7b, D2, T4b |
| | 398 | 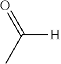 | | T7b, D2, T4b |
| | 399 | | | T7b |
| | 400 | | | E4 |
| | 401 | | | E4 |
| | 402 | | | E4, D2, T4 |
| | 405 | | | A4 |
| | 406 | | | A4 |
| | 407 | | | A4 |
| | 409 | 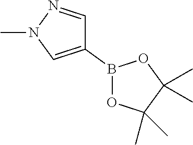 | | A1, B1b |
| | 410 | 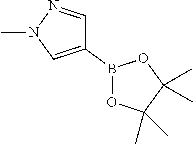 | | A1, B1b |
| | 411 | 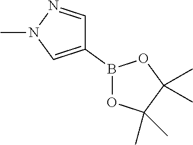 | | A1, B1b |
| | 412 | 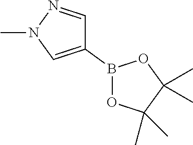 | | A1, B1b |
| | 413 | 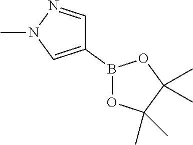 | | A1, B1b |
| | 414 | 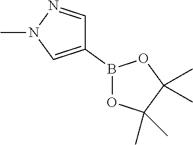 | | A1, B1b |
| | 415 | 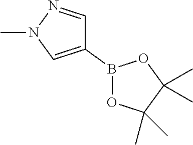 | | A1, B1b |

### Example 70: (R)-7-fluoro-N-(7-(1-methyl-1H-pyrazol-4-yl)-5-(1-(oxetan-3-yl)ethoxy)quinazolin-4-yl)benzo[d]thiazol-6-amine or(S)-7-fluoro-N-(7-(1-methyl-1H-pyrazol-4-yl)-5-(1-(oxetan-3-yl)ethoxy)quinazolin-4-yl)benzo[d]thiazol-6-amine

### Example 71: (S)-7-fluoro-N-(7-(1-methyl-1H-pyrazol-4-yl)-5-(1-(oxetan-3-yl)ethoxy)quinazolin-4-yl)benzo[d]thiazol-6-amine or (R)-7-fluoro-N-(7-(1-methyl-1H-pyrazol-4-yl)-5-(1-(oxetan-3-yl)ethoxy)quinazolin-4-yl)benzo[d]thiazol-6-amine

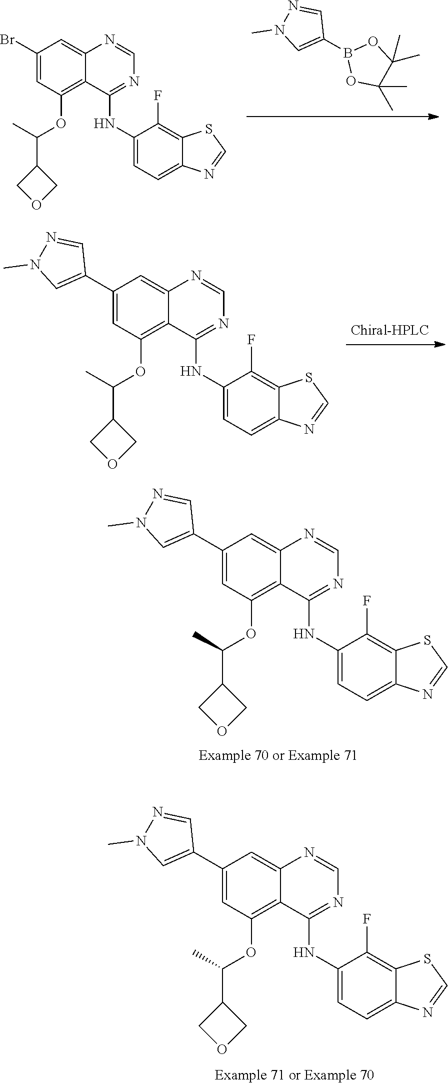

### Step 1: 7-fluoro-N-(7-(1-methyl-1H-pyrazol-4-yl)-5-(1-(oxetan-3-yl)ethoxy)quinazolin-4-yl)benzo[d]thiazol-6-amine

K₂CO₃ (60.8 mg, 441 μmol) was added to Pd(dppf)Cl₂·DCM (24.4 mg, 29.4 mmol). Intermediate BP-70 (140 mg, 294 μmol) and 1-methyl-4-(4,4,5,5-tetramethyl-1,3,2-dioxaborolan-2-yl)-1-1-pyrazole (73.2 mg, 352 μmol) in dioxane/H₂O (10 mL/2 mL) at rt. The resulting mixture was stirred at 100 degrees for 3 h. The mixture was diluted with EA 100 ml and washed with brine 50 mL*2, the organic layer was dried with Na₂SO₄ and concentrated under vacuum. The residue was purified by Prep-HPLC as following conditions: Column: XBridge Prep OBD C18 Column, 30-150 mm Sum; Mobile Phase A: Water (10 mmol/L NH₄HCO₃+0.1% NH₃·H₂O), Mobile Phase B. ACN, Flow rate: 60 mL/min; Gradient: 33 B to 53 B in 7 min; 254/220 nm to afford the title compound (40 mg) as a white solid. LC-MS: (ES, m/z): RT=0.581 min, m/z=477 [M+1].

### Step 2: Chiral Separation

The product of step 1 (40 mg, 83.9 μmol) in MeOH 10 mL was purified by Chiral-HPLC as following conditions: Column: CHIRALPAK IA, 2*25 cm, 5 μm; Mobile Phase A: Hex:DCM=3:1 (0.5% 2M NH₃-MeOH)-HPLC, Mobile Phase B: DCM:EtOH=9:1-HPLC; Flow rate: 20 mL/min; Gradient: 10% B to 10% B in 13 min; Wave Length: 220/254 nm; RT1 (min): 10.196; RT2 (min): 11.675 to afford:

Example 71, first eluting isomer, 13 mg as a white solid. LC-MS: (ES, m/z): RT=1.017 min, m/z=477 [M+1], chiral-HPLC: R=2.252, 1H NMR (400 MHz, DMSO-d6) δ 10.08 (s, 1H), 9.43 (d, J=1.4 Hz, 1H), 8.49 (d, J=7.4 Hz, 2H), 8.43 (t, J=8.3 Hz, 1H), 8.19 (s, 1H), 8.00 (d, J=8.8 Hz, 1H), 7.60 (s, 1H), 7.50 (s, 1H), 5.44 (p, J=6.1 Hz, 1H), 4.82 (td, J=8.1, 7.2, 2.5 Hz, 2H), 4.59 (t, J=5.9 Hz, 1H), 4.50 (t, J=5.9 Hz, 1H), 3.92 (s, 3H), 3.48-3.40 (m, 1H), 1.40 (d, J=5.9 Hz, 3H),

Example 70, second eluting isomer, 13 mg as a white solid. LC-MS: (ES, m/z): RT=1.018 min, m/z=477 [M+1], chiral-HPLC: R=2.772, 1H NMR (400 MHz, DMSO-d6) δ 10.08 (s, 1H), 9.43 (d, J=1.5 Hz, 1H), 8.49 (d, J=7.9 Hz, 2H), 8.43 (dd, J=8.8, 7.8 Hz, 1H), 8.19 (d, J=0.8 Hz, 1H), 8.00 (d, J=8.8 Hz, 1H), 7.60 (d, J=1.4 Hz, 1H), 7.50 (s, 1H), 5.47-5.40 (m, 1H), 4.86-4.77 (m, 2H), 4.59 (t, J=5.9 Hz, 1H), 4.50 (t, J=5.9 Hz, 1H), 3.92 (s, 3H), 3.43-3.33 (m, 1H), 1.40 (d, J=5.9 Hz, 3H),

### Example 133: (R)—N-(7-(1-methyl-1H-pyrazol-4-yl)-5-(1-(oxetan-3-yl)ethoxy)quinazolin-4-yl)benzo[d]thiazol-6-amine

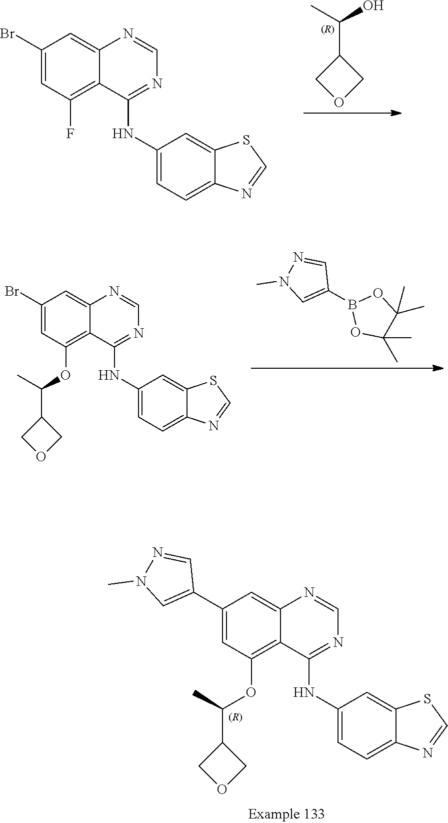

### Step 1: (R)—N-(7-bromo-5-(1-(oxetan-3-yl)ethoxy)quinazolin-4-yl)benzo[d]thiazol-6-amine

t-BuOK (1.96 g, 175 mmol) was added to Intermediate BP-59 (3.6 g, 9.59 mmol) and (1R)-1-(oxetan-3-yl)ethan-1-ol (1.95 g, 19.1 mmol) in THF at rt. The mixture was stirred at 80 degrees for 16 h. The mixture was diluted with EA 1000 mL and washed with brine 500 mL*2, the organic layer was dried with Na₂SO₄ and concentrated under vacuum. The residue was purified by a silica gel column with DCM:MeOH=15:1 to afford 2.35 g N-(1,3-benzothiazol-6-yl)-7-bromo-5-[(1R)-1-(oxetan-3-yl)ethoxy]quinazolin-4-amine as a white solid. LC-MS: (ES, m/z): RT=0.689 min, LCMS: m/z=457 [M+1]

### Step 2: (R)—N-(7-(1-methyl-1H-pyrazol-4-yl)-5-(1-(oxetan-3-yl)ethoxy)quinazolin-4-yl)benzo[d]thiazol-6-amine

K₂CO₃ (1.62 g, 11.8 mmol) was added to Pd(dppf)Cl₂·DCM (0.481 g, 0.59 mmol), the product of Step 1 (2.7 g, 5.90 mmol) and 1-methyl-4-(4,4,5,5-tetramethyl-1,3,2-dioxaborolan-2-yl)-1H-pyrazole (1.84 g, 8.85 mmol) in dioxane/H₂O (50 mL/15 mL) at rt. The mixture was heated to 80 degrees and stirred at this temperature under N₂ for 3 h. The mixture was diluted with EA 1000 mL and washed with brine 500 mL*2, the organic layer was dried with Na₂SO₄ and concentrated under vacuum. The residue was purified by a silica gel column with DCM:MeOH=15:1 to afford the title compound (1.3 g) as a white solid. LC-MS: (ES, m/z): RT=0.820 min, LCMS: m/z=459 [M+1]; 1H NMR (400 MHz, DMSO-d6) δ 10.17 (s, 1H), 9.33 (s, 1H), 8.76 (d, J=2.2 Hz, 1H), 8.51 (d, J=17.0 Hz, 2H), 8.18 (d, J=0.8 Hz, 1H), 8.09 (d, J=8.8 Hz, 1H), 7.79 (dd, J=8.9, 2.1 Hz, 1H), 7.58 (d, J=1.4 Hz, 1H), 7.48 (d, J=1.6 Hz, 1H), 5.41 (p, J=6.1 Hz, 1H), 4.90-4.80 (m, 2H), 4.65 (t, J=6.1 Hz, 1H), 4.56 (t, J=6.0 Hz, 1H), 3.92 (s, 3H), 3.54 (qd, J=7.9, 6.9, 2.0 Hz, 1H), 1.42 (d, J=6.0 Hz, 3H),

### Example 159: (S)—N-(5-((1-(dimethylamino)propan-2-yl)oxy)-7-(1-methyl-1H-pyrazol-4-yl)quinazolin-4-yl)benzo[d]thiazol-6-amine or (R)—N-(5-((1-(dimethylamino)propan-2-yl)oxy)-7-(1-methyl-1H-pyrazol-4-yl)quinazolin-4-yl)benzo[d]thiazol-6-amine

### Example 160: (R)—N-(5-((1-(dimethylamino)propan-2-yl)oxy)-7-(1-methyl-1H-pyrazol-4-yl)quinazolin-4-yl)benzo[d]thiazol-6-amine or (S)—N-(5-((1-(dimethylamino)propan-2-yl)oxy)-7-(1-methyl-1H-pyrazol-4-yl)quinazolin-4-yl)benzo[d]thiazol-6-amine

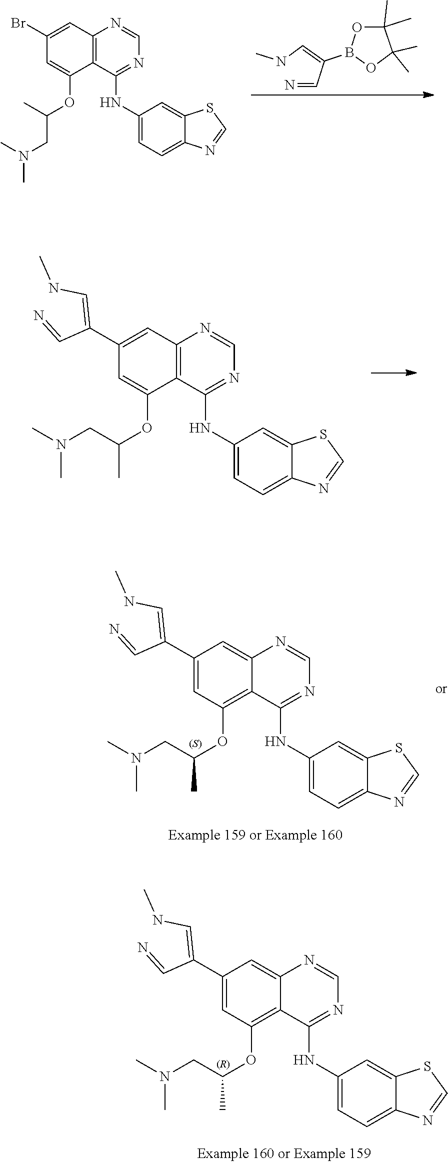

### Step 1. Rac-N-(5-((1-(dimethylamino)propan-2-yl)oxy)-7-(1-methyl-1H-pyrazol-4-yl)quinazolin-4-yl)benzo[d]thiazol-6-amine

To a 20 ml vial was charged with Methanesulfonato[9,9-dimethyl-4,5-bis(diphenylphosphino)xanthene][2′-amino-1,1′-biphenyl]palladium(II) dichloromethane adduct (8.80 mg, 9.27 μmol), 1-methyl-4-(4,4,5,5-tetramethyl-1,3,2-dioxaborolan-2-yl)-1H-pyrazole (38.6 mg, 0.185 mmol), Intermediate BP-70a (85 mg, 0.185 mmol) and Potassium phosphate (93 μl, 0.185 mmol). Sparged with nitrogen. Heated the reaction to 100 C. Filtered to remove insolubles and rinsed with min amount of DMSO. Purified by Prep-HPLC using a 0-40% 0.01% TFA Water/ACN gradient. To afford the title compound (78 mg, 0.161 mmol, 87% yield).

### Step 2: Chiral Separation

The product of Step 1 was Purified by Prep-Chiral-HPLC with following condition: Column: CHIRALPAK IH, 2*25 cm, 5 μm; Mobile Phase A: MTBE (0.5% 2M NH₃-MeOH)-HPLC, Mobile Phase B: EtOH-HPLC; Flow rate: 20 mL/min; Gradient: 10% B to 10% B in 12 min; Wave Length: 220/254 nm; RT1 (min): 8.146; RT2 (min): 10.738; Sample Solvent: EtOH-HPLC to afford:

Example 159, first Eluting isomer (149 mg) as an off-white solid. LC-MS: (ES, m/z): RT=0.630 min, LCMS: m/z=460.35 [M+1], Chiral-HPLC (ES): RT=1.962, 1H NMR (300 MHz, DMSO-d6) δ 10.62 (s, 1H), 9.32 (s, 1H), 8.83 (dd, J=5.1, 2.1 Hz, 1H), 8.53 (d, J=2.0 Hz, 2H), 8.18-8.06 (m, 2H), 7.80 (dd, J=8.8, 2.1 Hz, 1H), 7.55 (d, J=1.4 Hz, 1H), 7.39 (d, J=1.5 Hz, 1H), 5.11 (s, 1H), 3.92 (s, 3H), 3.34 (s, 1H), 3.10-2.96 (m, 1H), 2.26 (s, 6H), 1.51 (d, J=5.9 Hz, 3H),

Example 160, second eluting isomer (149.4 mg) as an off-white solid. LC-MS: (ES, m/z): RT=0.628 min, LCMS: m/z=460.35 [M+1], Chiral-HPLC (ES): RT=2.696, 1H NMR (300 MHz, DMSO-d6) δ 10.61 (s, 1H), 9.32 (s, 1H), 8.83 (dd, J=5.1, 2.1 Hz, 1H), 8.53 (s, 2H), 8.18-8.07 (m, 2H), 7.80 (dt, J=8.8, 1.6 Hz, 1H), 7.55 (d, J=1.4 Hz, 1H), 7.39 (d, J=1.6 Hz, 1H), 5.12 (s, 1H), 3.92 (s, 3H), 3.37 (s, 1H), 3.04 (s, 1H), 2.27 (d, J=4.2 Hz, 6H), 1.51 (d, J=5.9 Hz, 3H),

### Example A3: N-(7-(1-methyl-1H-pyrazol-4-yl)-5-((1-methylpiperidin-4-yl)oxy)quinazolin-4-yl)benzo[d]thiazol-5-amine

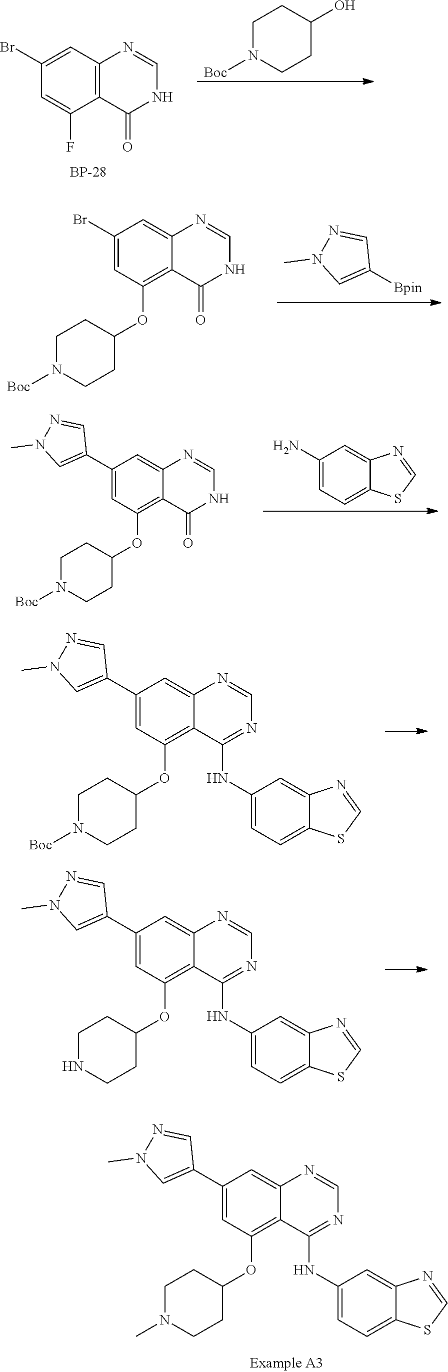

### Step 1: tert-butyl 4-((7-bromo-4-oxo-3,4-dihydroquinazolin-5-yl)oxy)piperidine-1-carboxylate

To a solution of tert-butyl 4-hydroxypiperidine-1-carboxylate (496.88 mg, 2.47 mmol, 1.2 eq) in DMF (2 mL) was added NaH (164.59 mg, 4.11 mmol, 60% purity, 2 eq) at 0° C. Intermediate BP-28 (500 mg, 2.06 mmol, 1 eq) was added and the reaction heated at 60° C. for 2 hrs. The reaction mixture was added into the H₂O (10 mL), filtered and the filter cake was collected to afford the title compound (900 mg, crude) as a yellow solid. LC-MS: (ES, m/z): RT=1.229 min, LC-MS: m/z=423.0 [M+1].

### Step 2: tert-butyl 4-((7-(1-methyl-1H-pyrazol-4-yl)-4-oxo-3,4-dihydroquinazolin-5-yl)oxy)piperidine-1-carboxylate

To a solution of tert-butyl 4-((7-bromo-4-oxo-3,4-dihydroquinazolin-5-yl)oxy)piperidine-1-carboxylate (700 mg, 1.65 mmol, 1 eq) and 1-methyl-4-(4,4,5,5-tetramethyl-1,3,2-dioxaborolan-2-yl)pyrazole (343.27 mg, 1.65 mmol, 1 eq) in DMSO (10 mL) and H₂O (2 mL) was added XPhos Pd G₂ (129.81 mg, 164.98 umol, 0.1 eq) and K₃PO₄ (700.40 mg, 3.30 mmol, 2 eq) at rt. The reaction stirred at 80° C. for 12 hrs under N₂. The mixture was concentrated in vacuum. The mixture was purified by prep-HPLC (column: Kromasil C18 (250*50 mm*10 um); mobile phase: [water (0.04% NH₃H₂O+10 mM NH₄HCO₃)-ACN]; B %: 20%-50%, 10 min) to afford the title compound (210 mg, 493.56 umol, 29.92% yield) as a yellow solid. LC-MS: (ES, m/z): RT=1.935 min, LC-MS: m/z=425.2 [M+1].

### Step 3: tert-butyl 4-((4-(benzo[d]thiazol-5-ylamino)-7-(1-methyl-1H-pyrazol-4-yl)quinazolin-5-yl)oxy)piperidine-1-carboxylate

To a solution of tert-butyl 4-((7-(1-methyl-1H-pyrazol-4-yl)-4-oxo-3,4-dihydroquinazolin-5-yl)oxy)piperidine-1-carboxylate (60 mg, 141.02 umol, 1 eq) in DCE (1 mL) was added PPh₃ (110.96 mg, 423.05 umol, 3 eq) and CCl₄ (130.15 mg, 846.10 umol, 81.34 uL, 6 eq). The reaction was heated at 75° C. for 2 hrs under N₂. 1,3-benzothiazol-5-amine (21.18 mg, 141.02 umol, 1 eq) was added to the reaction mixture at rt, then stirred at rt for 1 hr. The mixture was concentrated in vacuum. The residue was purified by prep-HPLC (column: Waters Xbridge Prep OBD C18 150*40 mm*10 um; mobile phase: [water (0.05% NH₃H₂O+10 mM NH₄HCO₃)-ACN]; B %: 35%-65%, 8 min) to afford the title compound (10 mg, 17.93 umol, 12.72% yield) as a white solid. LC-MS: (ES, m/z): RT=2.701 min, LC-MS: m/z=557.2 [M+1].

### Step 4: N-(7-(1-methyl-1H-pyrazol-4-yl)-5-(piperidin-4-yloxy)quinazolin-4-yl)benzo[d]thiazol-5-amine

A solution of tert-butyl 4-((4-(benzo[d]thiazol-5-ylamino)-7-(1-methyl-1H-pyrazol-4-yl)quinazolin-5-yl)oxy)piperidine-1-carboxylate (10 mg, 17.93 umol, 1 eq) in DCM (1 mL) and TFA (1 mL) was stirred at rt for 0.5 hr. The mixture was concentrated to afford the title compound (10 mg, crude, TFA Salt) as a yellow oil. LC-MS: (ES, m/z): RT=1.921 min, LC-MS: m/z=457.1 [M+1].

### Step 5: N-(7-(1-methyl-1H-pyrazol-4-yl)-5-((1-methylpiperidin-4-yl)oxy)quinazolin-4-yl)benzo[d]thiazol-5-amine

A solution of N-(7-(1-methyl-1H-pyrazol-4-yl)-5-(piperidin-4-yloxy)quinazolin-4-yl)benzo[d]thiazol-5-amine (10 mg, 17.50 umol, TFA Salt) in MeOH (3 mL) was adjusted to pH=8 with DIPEA (2.26 mg, 17.50 umol, 3.05 uL, 1 eq) then adjusted Ph=5 with AcOH (1.05 mg, 17.50 umol, 1.00 uL, 1 eq). HCHO (4.26 mg, 52.49 umol, 3.91 uL, 37% purity, 3 eq) was added to the reaction mixture, and stirred at 25° C. for 0.5 hr. NaBH₃CN (3.30 mg, 52.49 umol, 3 eq) was added to the reaction. The reaction mixture was stirred at 25° C. for 1 hr. The mixture was concentrated under vacuum and the residue purified by prep-HPLC (column: Waters Xbridge Prep OBD C18 150*40 mm*10 um; mobile phase: [water (0.05% NH₃H₂O+10 mM NH₄HCO₃)-ACN]; B %: 20%-50%, 8 min) to afford the title compound (2.1 mg, 4.27 umol, 24.38% yield) as an off-white solid. LC-MS: (ES, m/z): RT=1.570 min, LC-MS: m/z=471.1 [M+1]. ¹H NMR (400 MHz, DMSO-d6) δ 10.30 (s, 1H), 9.43 (s, 1H), 8.82 (s, 1H), 8.56 (s, 1H), 8.46 (s, 1H), 8.17 (t, 2H, J=8.8 Hz), 7.73 (d, 1H, J=8.8 Hz), 7.57 (s, 1H), 7.41 (s, 1H), 5.02 (s, 1H), 3.91 (s, 3H), 2.67 (s, 2H), 2.34 (s, 2H), 2.20 (s, 5H), 1.99-1.90 (m, 2H).

### Example A5: N4-(benzo[d]thiazol-6-yl)-N6-((3R,4S)-3-fluoro-1-methylpiperidin-4-yl)-7-(1-methyl-1H-pyrazol-3-yl)quinazoline-4,6-diamine or N4-(benzo[d]thiazol-6-yl)-N6-((3S,4R)-3-fluoro-1-methylpiperidin-4-yl)-7-(1-methyl-1H-pyrazol-3-yl)quinazoline-4,6-diamine

### Example A6: N4-(benzo[d]thiazol-6-yl)-N6-((3S,4R)-3-fluoro-1-methylpiperidin-4-yl)-7-(1-methyl-1H-pyrazol-3-yl)quinazoline-4,6-diamine or N4-(benzo[d]thiazol-6-yl)-N6-((3R,4S)-3-fluoro-1-methylpiperidin-4-yl)-7-(1-methyl-1H-pyrazol-3-yl)quinazoline-4,6-diamine

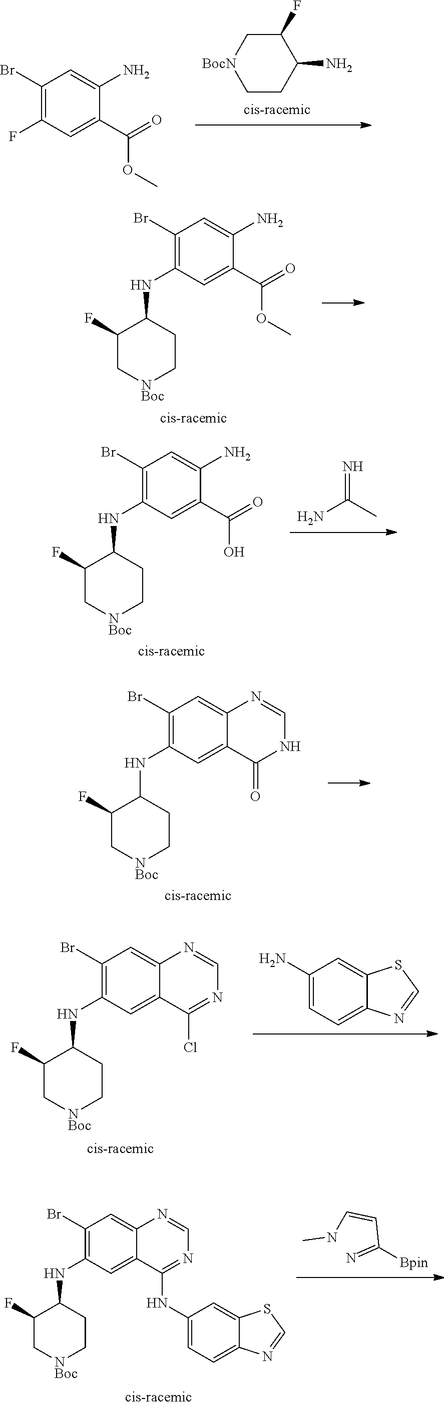

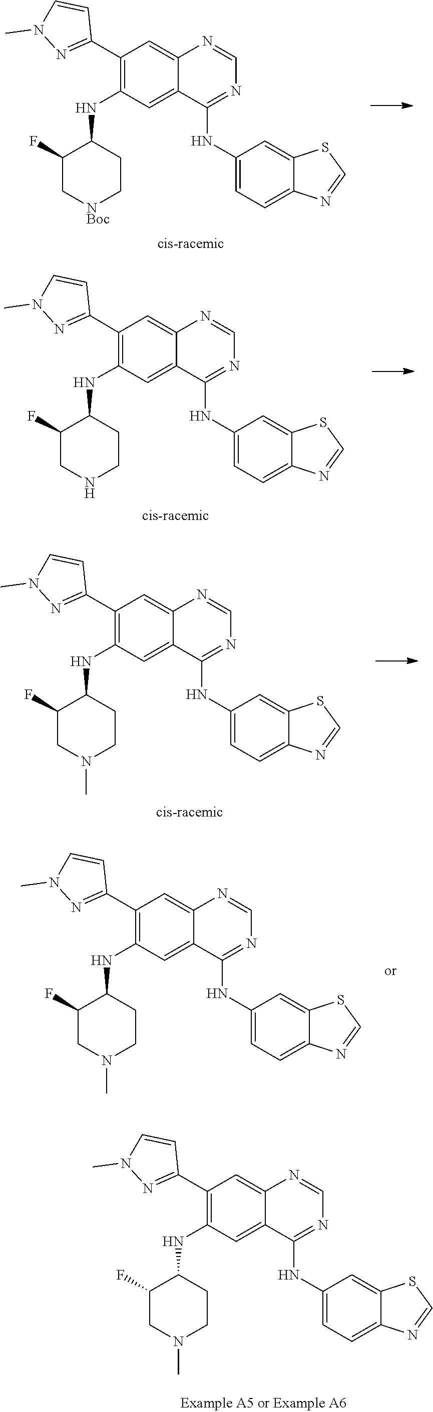 [SMILES: Cn1ccc(-c2cc3ncnc(Nc4ccc5ncsc5c4)c3cc2N[C@H]2CCN(C(=O)OC(C)(C)C)C[C@H]2F)n1 ; Cn1ccc(-c2cc3ncnc(Nc4ccc5ncsc5c4)c3cc2N[C@H]2CCNC[C@H]2F)n1 ; CN1CC[C@H](Nc2cc3c(Nc4ccc5ncsc5c4)ncnc3cc2-c2ccn(C)n2)[C@H](F)C1 ; CN1CC[C@H](Nc2cc3c(Nc4ccc5ncsc5c4)ncnc3cc2-c2ccn(C)n2)[C@H](F)C1 ; CN1CC[C@@H](Nc2cc3c(Nc4ccc5ncsc5c4)ncnc3cc2-c2ccn(C)n2)[C@@H](F)C1]

### Step 1: cis-rac-tert-butyl (3R,4S)-4-((4-amino-2-bromo-5-(methoxycarbonyl)phenyl)amino)-3-fluoropiperidine-1-carboxylate

Into a 20-mL pressure tank reactor purged and maintained with an inert atmosphere of nitrogen, was placed methyl 4-bromo-5-fluoro-2-nitrobenzoate (1000.00 mg, 3.597 mmol, 1.00 eq), cis-rac-tert-butyl (3R,4S)-4-amino-3-fluoropiperidine-1-carboxylate (785.06 mg, 3.597 mmol, 1.00 eq). TEA (1091.85 mg, 10.790 mmol, 3 eq), IPA (5.00 mL). The resulting solution was stirred for 12 hr at 100° C. The reaction was then quenched by the addition of 1 (0 mL of water/ice. The solids were filtered out. Mie resulting solution was extracted with 3×10 mL of EA concentrated under vacuum. The residue was purified by silica gel column, eluting with DCM/MeOH (20/1) to afford the title compound (1000 mg, 58.37%) as a light yellow solid. LC-MS: (ES, m/z): RT=0.931 min, LCMS: m/z=446 [M+1]

### Step 2: cis-rac-2-amino-4-bromo-5-(((3R,4S)-1-(tert-butoxycarbonyl)-3-fluoropiperidin-4-yl)amino)benzoic acid

Into a 20-mL pressure tank reactor purged and maintained with an inert atmosphere of nitrogen, was placed cis-rac-tert-butyl (3R,4S)-4-((4-amino-2-bromo-5-(methoxycarbonyl)phenyl)amino)-3-fluoropiperidine-1-carboxylate (1000.00 mg, 2.241 mmol, 1.00 eq), LiOH (107.31 mg, 4.481 mmol, 2 eq), MeOH (5.00 mL), H₂O (1.00 mL). The resulting solution was stirred for 1 hr. at 60° C. The pH value of the solution was adjusted to 3 with HCL (1 mol/L). The solids were filtered out. The resulting solution was extracted with 3×10 mL of EA concentrated under vacuum. The residue was purified by silica gel column, eluting with DCM/MeOH (20/1) to afford the title compound (900 mg) as a light yellow solid. LC-MS: (ES, m/z): RT=1.029 min, LCMS: m/z=432 [M+1]

### Step 3: cis-rac-tert-butyl (3R,4S)-4-((7-bromo-4-oxo-3,4-dihydroquinazolin-6-yl)amino)-3-fluoropiperidine-1-carboxylate

Into a 20-mL pressure tank reactor purged and maintained with an inert atmosphere of nitrogen, was placed cis-rac-2-amino-4-bromo-5-(((3R,4S)-1-(tert-butoxycarbonyl)-3-fluoropiperidin-4-yl)amino)benzoic acid (1000.00 mg, 2.313 mmol, 1.00 eq), ethyl alcohol (532.85 mg, 11.566 mmol, 5 eq) and formamide (5 mL). The resulting solution was stirred for 3 hr. at 80° C. The reaction was then quenched by the addition of 10 mL of water/ice. The solids were filtered out. The resulting solution was extracted with 3×10 mL of EA and concentrated under vacuum. The residue was purified by silica gel column, eluting with DCM/MeOH (20/1) to afford the title compound (900 mg, 88.16%) as a light yellow solid. LC-MS: (ES, m/z): RT=1.016 min, LCMS: m/z=441 [M+1]

### Step 4: cis-rac-tert-butyl (3R,4S)-4-((7-bromo-4-chloroquinazolin-6-yl)amino)-3-fluoropiperidine-1-carboxylate

Into a 20-mL pressure tank reactor purged and maintained with an inert atmosphere of nitrogen, was placed cis-rac-tert-butyl (3R,4S)-4-((7-bromo-4-oxo-3,4-dihydroquinazolin-6-yl)amino)-3-fluoropiperidine-1-carboxylate (400.00 mg, 0.906 mmol, 1.00 eq), ACN (5.00 mL). POCl₃ (694.91 mg, 4.532 mmol, 5 eq) and TEA (1375.80 mg, 13.596 mmol, 15 eq). The resulting solution was stirred for 1 hr. at 80° C. The reaction was then quenched by the addition of 10 mL of water/ice. The resulting solution was extracted with 3×10 mL of EA, concentrated under vacuum to afford the title compound (200 mg, 47.99%) as a yellow solid, LC-MS: (ES, m/z): RT=1.360 min, LCMS: m/z=460 [M+1]

### Step 5: cis-rac-tert-butyl (3R,4S)-4-((4-(benzo[d]thiazol-6-ylamino)-7-bromoquinazolin-6-yl)amino)-3-fluoropiperidine-1-carboxylate

Into a 20-mL pressure tank reactor purged and maintained with an inert atmosphere of nitrogen, was placed cis-rac-tert-butyl (3R,4S)-4-((7-bromo-4-chloroquinazolin-6-yl)amino)-3-fluoropiperidine-1-carboxylate (300.00 mg, 0.653 mmol, 1.00 eq), 6-benzothiazolamine (98.01 mg, 0.653 mmol, 1 eq), tert-butoxypotassium (146.45 mg, 1.305 mmol, 2 eq), THF (5.00 mL). The resulting solution was stirred for 1 hr. at 80° C. The reaction was then quenched by the addition of 10 mL of water/ice The solids were filtered away. The filtrate was extracted with EA (3×10 ml) and concentrated under vacuum. The residue was purified by silica gel chromatography (DCM/MeOH, 20/1) to afford the title compound (200 mg, 53.44%) as a light yellow solid. LC-MS: (ES, m/z): RT=1.656 min, LCMS: m/z=573 [M+1]

### Step 6: cis-rac-N4-(benzo[d]thiazol-6-yl)-N6-((3R,4S)-3-fluoropiperidin-4-yl)-7-(l-methyl-1H-pyrazol-3-yl)quinazoline-4,6-diamine

Into a 20-mL pressure tank reactor purged and maintained with an inert atmosphere of nitrogen, was placed cis-rac-tert-butyl (3R,4S)-4-((4-(benzo[d]thiazol-6-ylamino)-7-bromoquinazolin-6-yl)amino)-3-fluoropiperidine-1-carboxylate (200.00 mg, 0.349 mmol, 1.00 eq), potassium methaneperoxoate potassium (97.10 mg, 0.697 mmol, 2 eq), water (1.00 mL), Pd(dppf)Cl₂ (25.52 mg. 0.035 mmol, 0.1 eq), dioxane (5.00 mL). The resulting solution was stirred for 1 hr. at 80° C. The reaction was then quenched by the addition of 10 mL of water/ice. The solids were filtered out. The resulting solution was extracted with 3×10 mL of EA concentrated under vacuum The residue was purified by silica gel column, eluting with DCM/MeOH (20/1) to afford the title compound (150 mg. 74.84%) of tert-butyl (3R,4S)-4-[4-(1,3-benzothiazol-6-ylamino)-74I I-methylpyrazol-3-yl)quinazolin-6-yl]amino]-3-fluoropiperidine-1-carboxylate as a light yellow solid. LC-MS: (ES, m/z): RT=1.242 min, LCMS: m/z=575 [M+1]

### Step 7: cis-rac-N4-(benzo[d]thiazol-6-yl)-N6-((3R,4S)-3-fluoropiperidin-4-yl)-7-(1-methyl-1H-pyrazol-3-yl)quinazoline-4,6-diamine

Into a 20-mL pressure tank reactor purged and maintained with an inert atmosphere of nitrogen, was placed cis-rac-tert-butyl (3R,4S)-4-((4-(benzo[d]thiazol-6-ylamino)-7-(1-methyl-IH-pyrazol-3-yl)quinazolin-6-yl)amino)-3-fluoropiperidine-1-carboxylate (150.00 mg, 0.261 mmol, 1.00 eq), DCM (5.00 mL) and TFA (2.00 mL). The resulting solution was stirred for 1 hr. at 0° C. The resulting solution was concentrated under vacuum to afford the title compound (150 mg) as a solid. LC-MS: (ES, m/z): LCMS: m/z=475 [M+1]

### Step 8: cis-rac-N4-(benzo[d]thiazol-6-yl)-N6-((3R,4S)-3-fluoro-1-methylpiperidin-4-yl)-7-(1-methyl-1H-pyrazol-3-yl)quinazoline-4,6-diamine

Into a 20-mL pressure tank reactor purged and maintained with an inert atmosphere of nitrogen, was placed cis-rac-N4-(benzo[d]thiazol-6-yl)-N6-((3R,4S)-3-fluoropiperidin-4-yl)-7-(1-methyl-1H-pyrazol-3-yl)quinazoline-4,6-diamine (150.00 mg, 0.316 mmol, 1.00 eq), methanol (5.00 mL), HCHO (1.00 mL) and STAB (200.97 mg, 0.948 mmol, 3 eq). The resulting solution was stirred for 1 hr at 0° C. The reaction was quenched by the addition of 10 mL of water/ice. The solids were filtered out. The resulting solution was extracted with 3×10 mL of EA concentrated under vacuum. The residue was purified by silica gel column, eluting with DCM/MeOH (20/1) to afford the title compound (80 mg, 51.80%) as light yellow oil. LC-MS: (ES, m/z): RT=1.242 min, LCMS: m/z=575 [M+1]

### Step 9: Chiral Separation of Isomers

The isomers from step 8 were further purified by prep-HPLC prior to chiral separation: HPLC Column: XBridge Prep OBD C18 Column, 30*150 mm, 5 μm; Flow rate: 60 mL/min; Gradient: 25% B to 40% B in 8 min, 40% B; Wave Length: 254 nm; RT1 (min): 6.3 afforded cis-rac-N4-(1,3-benzothiazol-6-yl)-N6-[(3R,4S)-3-fluoro-1-methylpiperidin-4-yl]-7-(1-methyl-1H-pyrazol-3-yl)quinazoline-4,6-diamine (34.2 mg) as a yellow solid.

The isomers were separated by chiral HPLC: HPLC Column: CHIRAL ART Cellulose-SB, 4.6*100 mm, 3 μm: Mobile Phase A MtBE (0.1% DEA): MeOH=85:15: Flow rate: 1 mL/min: Gradient 0% B to 0% B; Injection Volume: 5 ul mL afforded:

Example A5, First Eluting Isomer: N4-(benzo[d]thiazol-6-yl)-N6-((3R,4S)-3-fluoro-1-methylpiperidin-4-yl)-7-(1-methyl-1H-pyrazol-3-yl)quinazoline-4,6-diamine or N4-(benzo[d]thiazol-6-yl)-N6-((3S,4R)-3-fluoro-1-methylpiperidin-4-yl)-7-(1-methyl-1H-pyrazol-3-yl)quinazoline-4,6-diamine (12.7 mg as a yellow solid. LC-MS: (ES, m/z): RT=0.990 min, LCMS: m/z=489[M+1], ¹H NMR (300 MHz, DMSO-d₆) δ 9.58 (s, 1H), 9.33 (s, 1H), 8.74 (d, J=2.0 Hz, 1H), 8.38 (d, J=17.3 Hz, 2H), 8.17-8.06 (m, 2H), 7.90 (dd, J=7.5, 2.3 Hz, 2H), 7.49 (s, 1H), 7.06 (d, J=2.4 Hz, 1H), 4.97 (s, OH), 3.97 (s, 4H), 2.36 (s, 3H), 2.06 (s, 2H), 1.84 (d, J=11.9 Hz, 2H),

Example A6, Second Eluting Isomer: N4-(benzo[d]thiazol-6-yl)-N6-((3S,4R)-3-fluoro-1-methylpiperidin-4-yl)-7-(1-methyl-1H-pyrazol-3-yl)quinazoline-4,6-diamine or N4-(benzo[d]thiazol-6-yl)-N6-((3R,4S)-3-fluoro-1-methylpiperidin-4-yl)-7-(1-methyl-1H-pyrazol-3-yl)quinazoline-4,6-diamine (12.8 mg) as a yellow % solid. LC-MS: (ES, m/z): RT=2.900 min, LCMS: m/z=489[M+1], ¹H NMR (300 MHz, DMSO-d₆) δ 9.58 (s, 1H), 9.33 (s, 1H), 8.74 (d, J=2.0 Hz, 1H), 8.38 (d, J=17.3 Hz, 2H), 8.17-8.06 (m, 2H), 7.90 (dd, J=7.5, 2.3 Hz, 2H), 7.49 (s, 1H), 7.06 (d, J=2.4 Hz, 1H), 4.97 (s, OH), 3.97 (s, 4H), 2.56 (s, 3H), 2.36 (s, 3H), 2.06 (s, 2H), 1.84 (d, J=11.9 Hz, 1H),

### Example A7: ((2S,4S)-4-((4-(benzo[d]thiazol-6-ylamino)-7-(1-methyl-1H-pyrazol-4-yl)quinazolin-5-yl)oxy)-1-methylpiperidin-2-yl)methanol

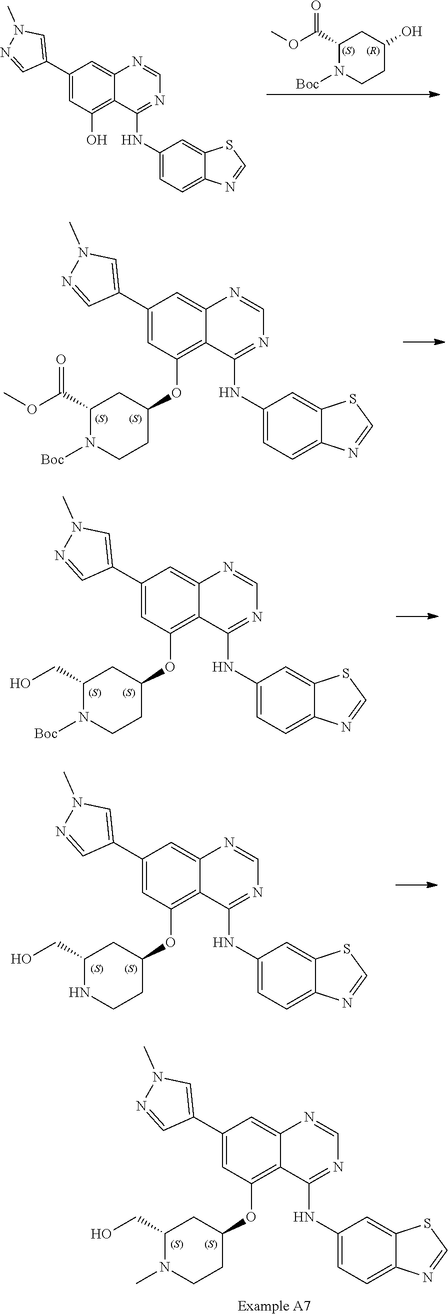

### Step 1: 1-(tert-butyl) 2-methyl (2S,4S)-4-((4-(benzo[d]thiazol-6-ylamino)-7-(1-methyl-1H-pyrazol-4-yl)quinazolin-5-yl)oxy)piperidine-1,2-dicarboxylate

To a solution of Intermediate BP-62 (200 mg, 534 μmol). 1-tert-butyl 2-methyl (2S)-4-hydroxypiperidine-1,2-dicarboxylate (207 mg, 801 umol) and PPh₃ (209 mg, 801 umol) in THF was added DBAD (184 mg, 801 umol) at 0° C. under N₂. The reaction was stirred for 2 hr. at rt. The mixture was diluted with water and extracted with EA. The organic layers were combined and dried over Na₂SO₄ and concentrated. The residue was purified by prep-TLC (DCM/MeOH (20:1)) to afford the title compound (210 mg, 64%) as a yellow solid. LC-MS: (ES, m/z): RT=0.729 min, LCMS: m/z=616.4 [M+1].

### Step 2: tert-butyl (2S,4S)-4-((4-(benzo[d]thiazol-6-ylamino)-7-(1-methyl-1H-pyrazol-4-yl)quinazolin-5-yl)oxy)-2-(hydroxymethyl)piperidine-1-carboxylate

To a solution of 1-(tert-butyl) 2-methyl (2S,4S)-4-((4-(benzo[d]thiazol-6-ylamino)-7-(1-methyl-1H-pyrazol-4-yl)quinazolin-5-yl)oxy)piperidine-1,2-dicarboxylate (210 mg, 341 μmol) in THF was added LiAlH₄ (12.9 mg, 341 umol) at 0° C. under N₂. The mixture was stirred for 2 hr. at 0° C. The mixture was diluted with water and extracted with EA. The organic layers were combined and dried over Na₂SO₄ and concentrated. The residue was purified by purified by Prep-TLC with DCM/MeOH (20:1) to afford the title compound (150 mg, 75%) as a yellow solid. LC-MS: (ES, m/z): RT=0.678 min, LCMS: m/z=588.4 [M+1],

### Step 3: ((2S,4S)-4-((4-(benzo[d]thiazol-6-ylamino)-7-(1-methyl-1H-pyrazol-4-yl)quinazolin-5-yl)oxy)piperidin-2-yl)methanol

To a solution of tert-butyl (2S,4S)-4-((4-(benzo[d]thiazol-6-ylamino)-7-(1-methyl-1H-pyrazol-4-yl)quinazolin-5-yl)oxy)-2-(hydroxymethyl)piperidine-1-carboxylate (150 mg, 255 μmol) in DCM (3 mL) was added TFA (1 mL) at RT. The mixture was stirred for 2 hr. at rt. The reaction was concentrated under vacuum. The crude title compound was used directly for next step (100 mg, 81%) as a brown solid. LC-MS: (ES, m/z): RT=0.519 min, LCMS: m/z=488.3 [M+1],

### Step 4: ((2S,4S)-4-((4-(benzo[d]thiazol-6-ylamino)-7-(1-methyl-1H-pyrazol-4-yl)quinazolin-5-yl)oxy)-1-methylpiperidin-2-yl)methanol

Into a reaction vessel was added ((2S,4S)-4-((4-(benzo[d]thiazol-6-ylamino)-7-(1-methyl-1H-pyrazol-4-yl)quinazolin-5-yl)oxy)piperidin-2-yl)methanol (50 mg, 102 umol), HCHO (15.2 mg, 510 mmol) and MeOH. The mixture was stirred for 30 min at rt. NaBH₃CN (32.1 mg, 510 mmol) was added into the reaction and stirred for 1 hr. at RT. The mixture was diluted with water and extracted with EA. The organic layers were combined and dried over Na₂SO₄ and concentrated. The residue was purified by prep-HPLC: Column: XBridge Shield RP18 OBD Column, 30*150 mm, 5 um; Mobile Phase A: Water (0.05% NH₃H₂O), Mobile Phase B: ACN; Flow rate: 60 mL/min; Gradient: 26 B to 34 B in 7 min; 254/220 nm to afford the title compound (10 mg) as a white solid. LC-MS: (ES, m/z): RT=1.122 min, LCMS: m/z=520.4 [M+1], 1H NMR (300 MHz, DMSO-d6) δ 10.18 (s, 1H), 9.32 (s, 1H), 8.81 (d, J=2.1 Hz, 1H), 8.53 (s, 1H), 8.44 (s, 1H), 8.16-8.05 (m, 2H), 7.79 (dd, J=8.9, 2.1 Hz, 1H), 7.55 (d, J=1.4 Hz, 1H), 7.38 (s, 1H), 5.32 (s, 1H), 4.56 (t, J=5.3 Hz, 1H), 3.90 (s, 3H), 2.74 (d, J=11.5 Hz, 1H), 2.21 (s, OH), 2.17 (s, 3H), 2.11 (s, 1H), 2.01 (d, J=11.3 Hz, 1H), 1.93-1.80 (m, 1H),

### Example A8: [(2R,4S)-4-({4-[(1,3-benzothiazol-6-yl)amino]-7-(1-methyl-1H-pyrazol-4-yl)quinazolin-5-yl}oxy)-1-methylpiperidin-2-yl]methanol

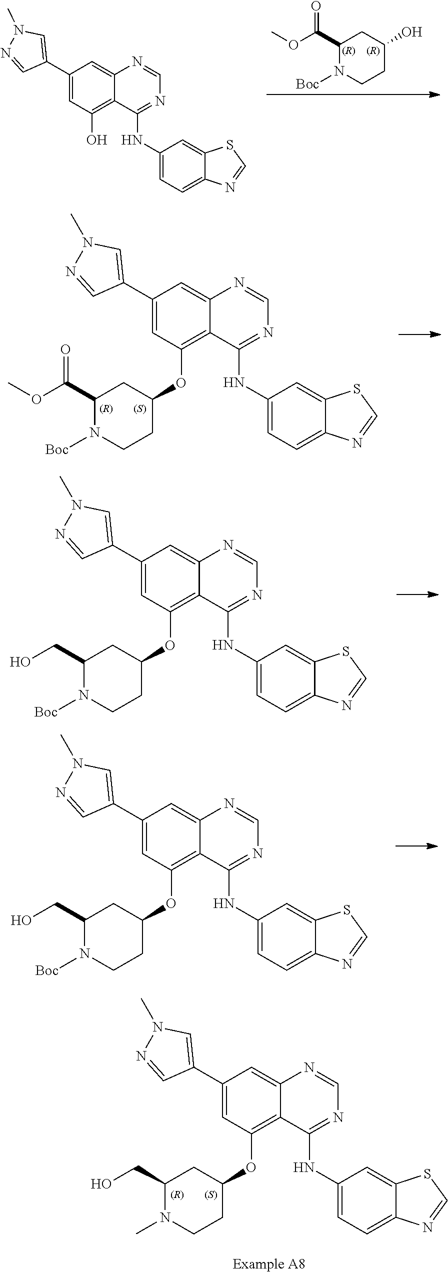

### Step I: 1-(tert-butyl) 2-methyl (2R,4S)-4-((4-(benzo[d]thiazol-6-ylamino)-7-(1-methyl-1H-pyrazol-4-yl)quinazolin-5-yl)oxy)piperidine-1,2-dicarboxylate

To a solution of Intermediate BP-62 (200 mg, 534 μmol), 1-tert-butyl 2-methyl (2R,4R)-4-hydroxypiperidine-1,2-dicarboxylate (207 mg, 801 umol) and PPh₃ (209 mg, 801 umol) in THF was added DTAD (184 mg, 801 umol) at 0° C. under N₂. The reaction was stirred for 2 hr. at rt. The mixture was diluted with water and extracted with EA. The organic layers were combined, dried over Na₂SO₄ and concentrated. The residue was purified by prep-TLC with DCM/MeOH (20:1) to afford the title compound (210 mg, 64%) as a yellow solid. LC-MS: (ES, m/z): RT=0.624 min, LCMS: m/z=616.15 [M+1].

### Step 2: tert-butyl (2R,4S)-4-((4-(benzo[d]thiazol-6-ylamino)-7-(1-methyl-1H-pyrazol-4-yl)quinazolin-5-yl)oxy)-2-(hydroxymethyl)piperidine-1-carboxylate

To a solution of 1-(tert-butyl) 2-methyl (2R,4S)-4-((4-(benzo[d]thiazol-6-ylamino)-7-(1-methyl-1H-pyrazol-4-yl)quinazolin-5-yl)oxy)piperidine-1,2-dicarboxylate (210 mg, 341 μmol) in THF was added LiAlH₄ (12.9 mg, 341 umol) at 0° C. under N₂. The reaction was stirred for 2 hr. at 0° C. The reaction was diluted with water and extracted with EA. The organic layers were combined and dried over Na₂SO₄ and concentrated. The residue was purified by Prep-TLC with DCM/MeOH (20:1) to afford the title compound (150 mg, 75%) of a yellow solid. LC-MS: (ES, m/z): RT=0.931 min, LCMS: m/z=588.2 [M+1],

### Step 3: tert-butyl (2R,4S)-4-((4-(benzo[d]thiazol-6-ylamino)-7-(1-methyl-1H-pyrazol-4-yl)quinazolin-5-yl)oxy)-2-(hydroxymethyl)piperidine-1-carboxylate

To a solution of tert-butyl (2R,4S)-4-((4-(benzo[d]thiazol-6-ylamino)-7-(1-methyl-1H-pyrazol-4-yl)quinazolin-5-yl)oxy)-2-(hydroxymethyl)piperidine-1-carboxylate (150 mg, 255 μmol) in DCM (3 mL) was added TFA (1 mL) at RT. The reaction was stirred for 2 hr. at rt. The resulting mixture was concentrated under vacuum to afford the crude product which was used directly for next step. LC-MS: (ES, m/z): RT=0.527 min, LCMS: m/z=488.3 [M+1].

### Step 4: ((2R,4S)-4-((4-(benzo[d]thiazol-6-ylamino)-7-(1-methyl-1H-pyrazol-4-yl)quinazolin-5-yl)oxy)-1-methylpiperidin-2-yl)methanol

Into a reaction tube was added tert-butyl (2R,4S)-4-((4-(benzo[d]thiazol-6-ylamino)-7-(1-methyl-1H-pyrazol-4-yl)quinazolin-5-yl)oxy)-2-(hydroxymethyl)piperidine-1-carboxylate (50 mg, 102 umol) HCHO (15.2 mg, 510 mmol) and MeOH at rt. The mixture was stirred for 30 min at rt. NaBH₃CN (32.1 mg, 510 mmol) was added into the mixture and stirred for 1 hr. at rt. The mixture was diluted with water and extracted with EA. The organic layers were combined and dried over Na₂SO₄ and concentrated. The residue was purified by prep-HPLC: Column: XBridge Prep OBD C18 Column, 30×150 mm Sum; Mobile Phase A: Water (10 mmol/L NH₄HCO₃+0.1% NH₃·H₂O), Mobile Phase B: ACN; Flow rate: 60 mL/min; Gradient: 23 B to 33 B in 7 min; 254/220 nm to afford the title compound (20 mg) as a light-yellow solid. LC-MS: (ES, m/z): RT=1.172 min, LCMS: m/z=502.4 [M+1], 1H NMR (300 MHz, DMSO-d6) δ 10.39 (s, 1H), 9.31 (s, 1H), 8.93 (d, J=2.1 Hz, 1H), 8.56 (s, 1H), 8.45 (s, 1H), 8.15-8.09 (m, 2H), 7.57 (s, 1H), 7.56 (s, 1H), 7.44 (s, 1H), 5.02-4.94 (m, 1H), 4.52 (t, J=5.5 Hz, 1H), 3.91 (s, 3H), 3.58-3.50 (m, 1H), 3.50-3.39 (m, 2H), 2.95-2.91 (m, 1H), 2.49-2.48 (m, 5H), 2.25-2.12 (m, 1H), 1.91-1.89 (m, 2H),

### Example A9, N-(5-(((1s,4s)-4-aminocyclohexyl)oxy)-7-(1-methyl-1H-pyrazol-4-yl)quinazolin-4-yl)benzo[d]thiazol-6-amine

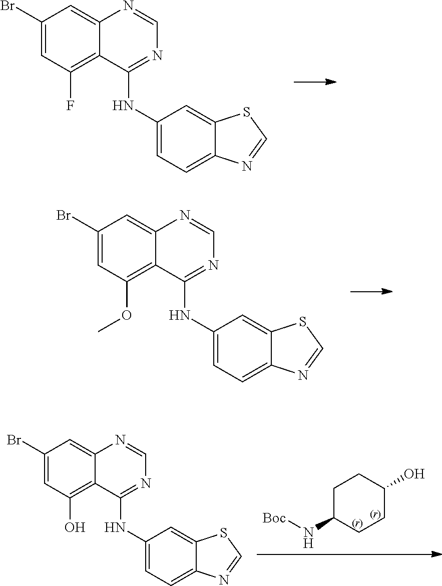

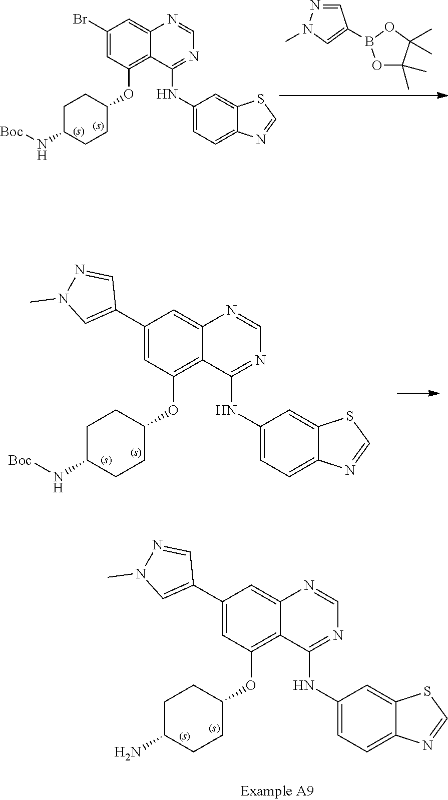

### Step 1: N-(7-bromo-5-methoxyquinazolin-4-yl)benzo[d]thiazol-6-amine

To a stirred solution of Intermediate BP-59 in MeOH/1,4-dioxane (20 mL) was added Cs₂CO₃ (2.7 g, 10.6 mmol) under N₂. The mixture was stirred at 110° C. for 2 hr. After cooling to rt, the reaction was extracted with DCM and concentrated under vacuum to afford the title compound (1.5 g, 72%) as yellow solid.

### Step 2: 4-(benzo[d]thiazol-6-ylamino)-7-bromoquinazolin-5-ol

To a stirred solution of N-(7-bromo-5-methoxyquinazolin-4-yl)benzo[d]thiazol-6-amine (100 mg, 258 μmol) in Pyridine (2 mL) was added pyridine hydrochloride (147 mg, 1.28 mmol) under nitrogen atmosphere. The resulting mixture was stirred at 110° C. for 2 hr. After cooling to rt, the reaction was filtered and the filter cake was collected. The crude was dissolved in MeOH and adjust PH=7.5 with TEA, then extracted with DCM to afford the title compound (80 mg, 83%) as yellow solid. LC-MS: (ES, m/z): RT=0.614 min, LCMS: m/z=373 [M+1].

### Step 3: tert-butyl ((1s,4s)-4-((4-(benzo[d]thiazol-6-ylamino)-7-bromoquinazolin-5-yl)oxy)cyclohexyl)carbamate

To a solution of 4-(benzo[d]thiazol-6-ylamino)-7-bromoquinazolin-5-ol (200 mg, 535 μmol) and tert-butyl N-[(1r,4r)-4-hydroxycyclohexyl]carbamate (230 mg, 1.07 mmol) in THF (3 mL) were added di-tert-butyl (E)-diazene-1,2-dicarboxylate (246 mg, 1.07 mmol) and triphenylphosphane (168 mg, 642 μmol) at 0° C. under nitrogen atmosphere. The mixture was stirred at 25° C. for 16 hr. The reaction was extracted with DCM and H₂O. The organic layer was concentrated under vacuum and the residue was purified by prep-TLC (7% MeOH in DCM) to afford the title compound (250 mg, 81%) as yellow solid. LC-MS: (ES, m/z): RT=0.800 min, LCMS: m/z=570 [M+1]

### Step 4: tert-butyl((1s,4s)-4-((4-(benzo[d]thiazol-6-ylamino)-7-(1-methyl-1H-pyrazol-4-yl)quinazolin-5-yl)oxy)cyclohexyl)carbamate

tert-butyl ((1s,4s)-4-((4-(benzo[d]thiazol-6-ylamino)-7-bromoquinazolin-5-yl)oxy)cyclohexyl)carbamate (90 mg, 157 μmol), 1-methyl-4-(4,4,5,5-tetramethyl-1,3,2-dioxaborolan-2-yl)-1H-pyrazole (39.1 mg, 188 μmol), 1,4-dioxane/H₂O (2 mL), tetrakis(triphenylphosphane) palladium (9.07 mg, 7.85 μmol) and dipotassium carbonate (43.3 mg, 314 μmol) were combined in a reaction vessel under nitrogen atmosphere. The mixture was stirred at 100° C. for 2 hr. The reaction was extracted with DCM and purified by Prep-TLC (DCM/MeOH=10:1) to afford the title compound (50 mg, 55%) as yellow solid. LC-MS: (ES, m/z): RT=0.658 min, LCMS: m/z=471 [M+1]

### Step 5: N-(5-(((1s,4s)-4-aminocyclohexyl)oxy)-7-(1-methyl-1H-pyrazol-4-yl)quinazolin-4-yl)benzo[d]thiazol-6-amine

To tert-butyl((1s,4s)-4-((4-(benzo[d]thiazol-6-ylamino)-7-(1-methyl-1H-pyrazol-4-yl)quinazolin-5-yl)oxy)cyclohexyl)carbamate (80 mg, 139 μmol) in DCM (1 mL) was added TFA (1 mL). The resulting mixture was stirred at 25° C. for 2 hrs. The reaction was concentrated under vacuum and purified by Prep-HPLC (Column: XBridge Shield RP18 OBD Column, 30*150 mm, Sum; Mobile Phase A: Water (0.05% NH₃H₂O), Mobile Phase B: ACN; Flow rate: 60 mL/min; Gradient: 30% B to 37% B in 8 min; Wave Length: 254/220 nm) to afford the title compound (30 mg, 46%) as white solid. LC-MS: (ES, m/z): RT=1.015 min, LCMS: m/z=472 [M+1], ¹H NMR (300 MHz, DMSO-d6) δ 10.28 (s, 1H), 9.34 (s, 1H), 8.89 (d, J=2.3 Hz, 1H), 8.54 (s, 1H), 8.47 (s, 1H), 8.18-8.07 (m, 2H), 7.81 (dd, J=8.8, 2.2 Hz, 1H), 7.56 (d, J=1.4 Hz, 1H), 7.38 (s, 1H), 5.11 (s, 1H), 3.92 (s, 3H), 2.91 (s, 1H), 2.24 (d, J=24.8 Hz, 2H), 1.87 (s, 2H), 1.74 (s, 2H), 1.48 (d, J=10.9 Hz, 2H),

### Example A10: N-(5-(((3S,4R)-4-fluorotetrahydrofuran-3-yl)oxy)-7-(1-methyl-1H-pyrazol-4-yl)quinazolin-4-yl)benzo[d]thiazol-6-amine

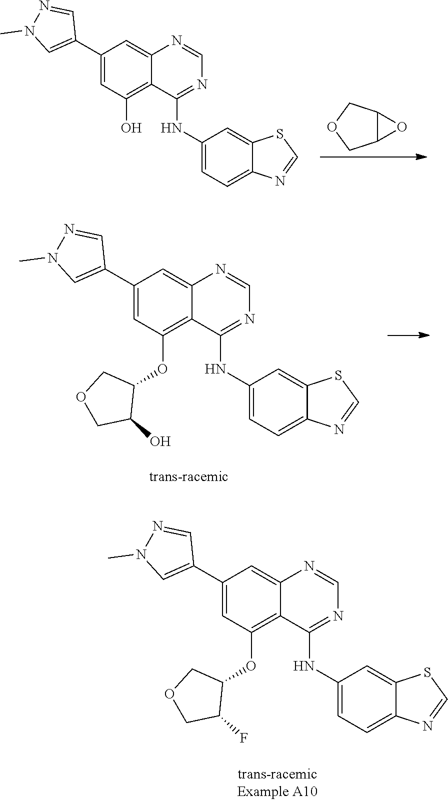

### Step 1: rac-(3S,4S)-4-((4-(benzo[d]thiazol-6-ylamino)-7-(1-methyl-1H-pyrazol-4-yl)quinazolin-5-yl)oxy)tetrahydrofuran-3-ol

To a mixture of Intermediate BP-62, 3,6-dioxabicyclo[3.1.0]hexane (91.2 mg, 1.06 mmol), and ACN (8 mL) was added 2 N aqueous NaOH (2 mL). The mixture was heated to 100° C. for 12 h. After cooling to rt, the mixture was concentrated. The residue was purified by prep-TLC (DCM:MeOH=10:1) to afford the title compound (150 mg) as a light yellow solid. LC-MS: (ES, m/z): RT=0.712 min, LCMS: m/z=461 [M+1].

### Step 2: rac-N-(5-(((3S,4R)-4-fluorotetrahydrofuran-3-yl)oxy)-7-(1-methyl-1H-pyrazol-4-yl)quinazolin-4-yl)benzo[d]thiazol-6-amine

To a solution of rac-(3S,4S)-4-((4-(benzo[d]thiazol-6-ylamino)-7-(1-methyl-1H-pyrazol-4-yl)quinazolin-5-yl)oxy)tetrahydrofuran-3-ol (100 mg, 217 μmol) in DCM (8 mL), was added DAST (69.9 mg, 434 μmol) at −78° C. The mixture was stirred at −78° C. for 0.5 hr. then slowly warmed to 40° C. After 12 hr. the mixture was quenched with NaHCO₃ (30 mL), extracted with DCM (20 mL*3) and washed with brine (10 mL). The organic layer was dried with Na₂SO₄ and concentrated under vacuum. The residue was purified by prep-HPLC (Column: XBridge Shield RP18 OBD Column, 30*150 mm, 5 μm; Mobile Phase A: Water (10 mmol/L NH₄HCO₃₀+0.1% NH₃·H₂O), Mobile Phase B: ACN; Flow rate: 60 mL/min; Gradient: 50% B to 85% B in 7 min, 85% B; Wave Length: 254/220 nm) to afford the title compound (18 mg, 18%) as an off-white solid. LC-MS: (ES, m/z): RT=1.239 min, LCMS: m/z=463 [M+1], ¹H NMR (300 MHz, DMSO-d6) δ ppm 10.20 (s, 1H), 9.31 (s, 1H), 8.94 (t, J=2.6 Hz, 1H), 8.59 (d, J=1.1 Hz, 1H), 8.48 (s, 1H), 8.18 (s, 1H), 8.10 (d, J=8.8 Hz, 1H), 7.72 (dd, J=8.9, 2.2 Hz, 1H), 7.62 (d, J=1.4 Hz, 1H), 7.39 (s, 1H), 5.83 (t, J=4.0 Hz, 1H), 5.64 (tq, J=11.1, 5.8 Hz, 1H), 4.44 (dd, J=9.3, 6.4 Hz, 1H), 4.26 (d, J=11.7 Hz, 1H), 4.22-4.07 (m, 1H), 4.06-3.93 (m, 1H), 3.91 (s, 3H),

### Example A11: 4-[(1,3-benzothiazol-6-yl)amino]-5-(oxan-4-yloxy)quinazolin-7-yl 4-methylpiperazine-1-carboxylate

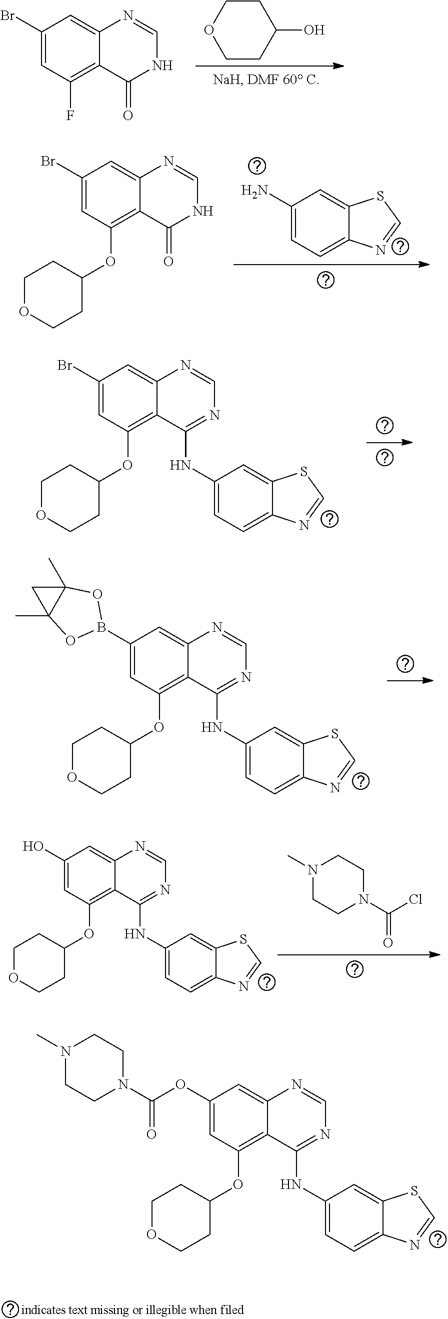

### Step 1: 7-bromo-5-(oxan-4-yloxy)-3,4-dihydroquinazolin-4-one

To a solution of Intermediate BP-28 (335.8 mg, 3.29 mmol) in DMF (10 ml) was added NaH (131.6 mg, 3.29 mmol) at 0° C. and stirred at rt for 1 h. 7-bromo-5-fluoroquinazolin-4(3H)-one (400 mg, 1.65 mmol) was added at rt and warmed to 60° C. for 4 h. The reaction mixture was diluted with water, and extracted with EA and saturated brine. The organic layer was dried over Na₂SO₄, filtered, evaporated, and purified by column chromatography (PE:EA=2:1) to afford the title compound (300 mg, 56.1%) as a yellow solid. LC-MS: (ES, m/z): RT=1.393 min, LCMS: m/z=325[M+1].

### Step 2: N-(1,3-benzothiazol-6-yl)-7-bromo-5-(oxan-4-yloxy)quinazolin-4-amine

To a solution of 7-bromo-5-(oxan-4-yloxy)-3,4-dihydroquinazolin-4-one (300 mg, 922 umol) in DCE (5 mL) was added PPh₃ (414 mg, 2.76 mmol) and CCl₄ (851 mg, 5.53 mmol). The mixture was stirred at 80° C. for 2 hrs under N₂. The reaction mixture was cooled to 25° C. A solution of 1,3-benzothiazol-6-amine (414 mg, 2.76 mmol) in DCE (1 mL) was added, the reaction was stirred at 25° C. for 1 hr. under N₂. The mixture was diluted with H₂O and EA. A solid was collected by filtration and the filter cake was dried under reduced pressure to afford the title (160 mg, 38.0%) as yellow solid. LC-MS: (ES, m/z): RT=2.128 min, LCMS: m/z=457[M+1].

### Step 3: N-(5-((tetrahydro-2H-pyran-4-yl)oxy)-7-(4,4,5,5-tetramethyl-1,3,2-dioxaborolan-2-yl)quinazolin-4-yl)benzo[d]thiazol-6-amine

To a solution of N-(1,3-benzothiazol-6-yl)-7-bromo-5-(oxan-4-yloxy)quinazolin-4-amine (160 mg, 349 umol), 4,4,5,5-tetramethyl-2-(4,4,5,5-tetramethyl-1,3,2-dioxaborolan-2-yl)-1,3,2-dioxaborolane (177 mg, 698 umol), Pd(dppf)Cl₂ (28.4 mg, 34.9 umol), KOAc (101 mg, 1.04 mmol) in DMSO (5 ml) were added under nitrogen and warmed to 80° C. for 3 h. The reaction mixture was diluted with water, filtered and the filter cake was dried under reduced pressure to afford the title compound N-(5-((tetrahydro-2H-pyran-4-yl)oxy)-7-(4,4,5,5-tetramethyl-1,3,2-dioxaborolan-2-yl)quinazolin-4-yl)benzo[d]thiazol-6-amine (90 mg, 61.2%) as a brown solid. LC-MS: (ES, m/z): RT=0.831 min, LCMS: m/z=423[M+1].

### Step 4: 4-[(1,3-benzothiazol-6-yl)amino]-5-(oxan-4-yloxy)quinazolin-7-ol

To a solution of N-(5-((tetrahydro-2H-pyran-4-yl)oxy)-7-(4,4,5,5-tetramethyl-1,3,2-dioxaborolan-2-yl)quinazolin-4-yl)benzo[d]thiazol-6-amine (90 mg, 213 umol) in H₂O (2 mL) was added H₂O₂ (0.2 ml) at 0° C. The mixture was stirred at rt for 2 hrs. The reaction mixture was filtered, and the filter cake was concentrated under reduced pressure to afford the title compound (50 mg, 59.5%) as grey solid. LC-MS: (ES, m/z): RT=0.938 min, LCMS: m/z=395[M+1].

### Step 5: 4-(benzo[d]thiazol-6-ylamino)-5-((tetrahydro-2H-pyran-4-yl)oxy)quinazolin-7-yl 4-methylpiperazine-1-carboxylate

To a solution of 4-[(1,3-benzothiazol-6-yl)amino]-5-(oxan-4-yloxy)quinazolin-7-ol (50 mg, 126 umol), Cs₂CO₃ (82.1 mg, 252 umol) in acetone (3 ml), 4-methylpiperazine-1-carbonyl chloride (20.4 mg, 126 umol) was added at 0° C. and warmed to RT for 3 h. The reaction mixture was diluted with H₂O, extracted with EA and washed with brine. The organic layer was dried over Na₂SO₄, filtered, evaporated, and purified by column chromatography (DCM:MeOH=10:1) and Prep-HPLC (Column: XBridge Shield RP18 OBD Column, 30*150 mm, 5 μm; Mobile Phase A: Water (0.05% NH₃H₂O), Mobile Phase B: ACN; Flow rate: 60 mL/min; Gradient: 31% B to 39% B in 8 min; Wave Length: 254/220 nm, to afford 4-[(1,3-benzothiazol-6-yl)amino]-5-(oxan-4-yloxy)quinazolin-7-yl 4-methylpiperazine-1-carboxylate (13 mg, 19.8%) as a white solid. LC-MS: (ES, m/z): RT=1.570 min, LCMS: m/z=521 [M+1], ¹H NMR (400 MHz, DMSO-d6) ¹H δ 10.25 (s, 1H), 9.33 (s, 1H), 8.90 (d, J=2.2 Hz, 1H), 8.59 (s, 1H), 8.12 (d, J=8.8 Hz, 1H), 7.79 (dd, J=8.8, 2.2 Hz, 1H), 7.16 (dd, J=20.6, 2.1 Hz, 2H), 5.04 (tt, J=8.7, 3.9 Hz, 1H), 3.95 (dt, J=11.9, 4.1 Hz, 2H), 3.68-3.52 (m, 4H), 3.50 (d, J=16.2 Hz, 2H), 2.42-2.38 (m, 4H), 2.26-2.20 (m, 5H), 1.99-1.87 (m, 2H),

### Example A16: N-(7-(2-(4-methylpiperazin-1-yl)ethoxy)-5-((tetrahydro-2H-pyran-4-yl)oxy)quinazolin-4-yl)benzo[d]thiazol-6-amine

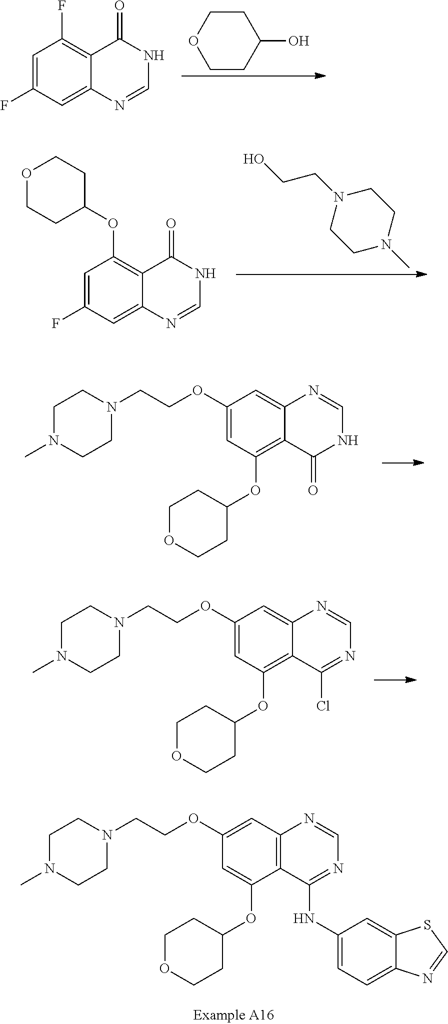

### Step 1: 7-fluoro-5-(oxan-4-yloxy)-3,4-dihydroquinazolin-4-one

To a mixture of oxan-4-ol (5.01 g, 49.1 mmol, 3.00 eq) in DMF (50 mL) was added NaH (981 mg, 40.9 mmol, 2.50 eq) at 0° C. The reaction mixture was stirred at 0° C. for 15 min, then 5,7-difluoro-3,4-dihydroquinazolin-4-one (3 g, 16.4 mmol, 1.00 eq) was added to the reaction mixture. The reaction was stirred at 25° C. for 16 hrs. The reaction mixture was added to ice water. The resulting solution was extracted with 2×50 mL of EA and the organic layers were washed with 20 mL of brine. The mixture was dried over anhydrous sodium sulfate and concentrated under vacuum. The crude product was purified by column chromatography (EA in PE=0% to 60%), to afford the title compound (4 g, 92.3%) as a yellow solid. LC-MS: (ES, m/z): RT=0.438 min, LCMS: m/z=265 [M+1]

### Step 2: 7-(2-(4-methylpiperazin-1-yl)ethoxy)-5-((tetrahydro-2H-pyran-4-yl)oxy)quinazolin-4(3H)-one

To a mixture of 7-fluoro-5-(oxan-4-yloxy)-3,4-dihydroquinazolin-4-one (4 g, 15.1 mmol, 1.00 eq) in THF (30 mL) was added 2-(4-methylpiperazin-1-yl)ethan-1-ol (4.35 g, 30.2 mmol, 2.00 eq) and BuOK (5.07 g, 45.3 mmol, 3.00 eq). The reaction was stirred at 80° C. for 24 hr. then cooled to room temperature. The resulting solution was diluted with 40 mL of water. The resulting solution was extracted with 3×50 mL of EA and the organic layers combined. The resulting mixture was washed with 30 mL of brine, dried over anhydrous sodium sulfate and concentrated under vacuum. The crude product was purified by column chromatography (DCM in MeOH=0% to 10%), to afford the title as a off-white solid. LC-MS: (ES, m/z): RT=1.202 min, LCMS: m/z=389 [M+1]

### Step 3: 4-chloro-7-(2-(4-methylpiperazin-1-yl)ethoxy)-5-((tetrahydro-2H-pyran-4-yl)oxy)quinazoline

To a solution of 7-(2-(4-methylpiperazin-1-yl)ethoxy)-5-((tetrahydro-2H-pyran-4-yl)oxy)quinazolin-4(3H)-one (1.5 g, 3.86 mmol, 1.00 eq) in DCE was added DIEA (2.98 g, 23.1 mmol, 6.00 eq) and POCl₃ (2.95 g, 19.3 mmol, 5.00 eq) under nitrogen. The mixture was stirred at 80° C. for 3 hr. The reaction mixture was cooled to room temperature and adjusted to pH=8 by slow addition of 5 N NaHCO₃. The aqueous solution was extracted with DCM (3×50 mL), the combined organic layers were washed with brine, dried over Na₂SO₄, filtered and concentrated. The resulting dark oil was purified by column chromatography eluting with 100% DCM to afford the title compound (800 mg, yield: 50.9%) as a off-white solid.

### Step 4: N-(7-(2-(4-methylpiperazin-1-yl)ethoxy)-5-((tetrahydro-2H-pyran-4-yl)oxy)quinazolin-4-yl)benzo[d]thiazol-6-amine

To a mixture of 4-chloro-7-(2-(4-methylpiperazin-1-yl)ethoxy)-5-((tetrahydro-2H-pyran-4-yl)oxy)quinazoline (500 mg, 921 μmol) in IPA (15 mL) was added 1,3-benzothiazol-6-amine (138 mg, 921 μmol) and TsOH (158 mg, 921 μmol). The mixture was stirred at 100° C. for 2 hr. The mixture was extracted with DCM (20 mL*3) and washed with brine (10 mL). The organic layer was dried with Na₂SO₄ and concentrated under vacuum. The residue was purified by prep. HPLC (Column: XBridge Prep OBD C18 Column, 30×150 mm 5 um; Mobile Phase A: Water (10 mmol/L NH₄HCO₃+0.1% NH₃·H₂O), Mobile Phase B: ACN; Flow rate: 60 mL/min; Gradient: 26 B to 34 B in 7 min; 254/220 nm) to afford the title compound (25 mg, 18.9%) as a white solid. LC-MS: (ES, m/z): RT=1.241 min, LCMS: m/z=521 [M+1], ¹H NMR (300 MHz, DMSO-d6) δ ppm 10.15 (s, 1H), 9.31 (s, 1H), 8.88 (d, J=2.2 Hz, 1H), 8.52 (s, 1H), 8.09 (d, J=8.8 Hz, 1H), 7.75 (dd, J=8.8, 2.2 Hz, 1H), 6.87 (dd, J=12.7, 2.2 Hz, 2H), 5.04 (dt, J=9.5, 4.9 Hz, 1H), 4.23 (t, J=5.7 Hz, 2H), 3.94 (dt, J=11.6, 4.0 Hz, 2H), 3.65-3.51 (m, 2H), 3.34 (m, 2H), 2.73 (t, J=5.7 Hz, 2H), 2.52 (m, 2H), 2.33 (s, 3H), 2.23 (d, J=12.5 Hz, 3H), 2.16 (s, 3H), 1.99-1.81 (m, 2H),

### Example A17: 4-(4-(benzo[d]thiazol-6-ylamino)-5-((1-methylpiperidin-4-yl)oxy)quinazolin-7-yl)-2-(difluoromethyl)-1-methyl-1,2-dihydro-3H-pyrazol-3-one

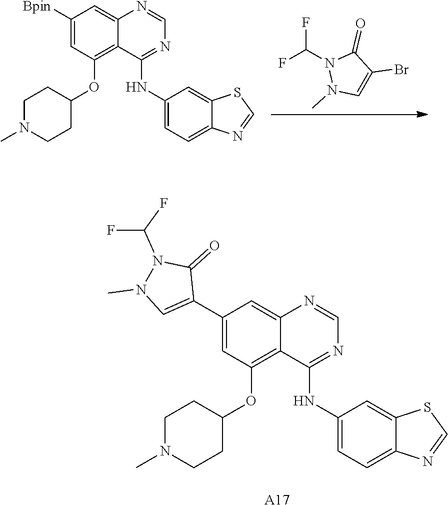

To a solution of Intermediate BP-053 (65.7 mg, 0.2898 mmol, 1.00 eq) in 1,4-dioxane/H2O was added intermediate BP-73 (150 mg, 0.2898 mmol, 1.00 eq), K₂CO₃ (79.9 mg, 0.57 mmol, 2.00 eq), Pd(dppf)Cl₂ (9.43 mg, 0.01 mmol, 0.05 eq) under nitrogen. The mixture was stirred at 80° C. for 3 hr. then cooled to room temperature. The resulting solution was diluted with 20 mL of water, extracted with 2×20 mL of EA, the organic layers combined and washed with 20 mL of brine. The mixture was dried over anhydrous sodium sulfate and concentrated under vacuum. The crude product was purified by Prep-HPLC with the following conditions: Column: XBridge Prep OBD C18 Column, 30×150 mm Sum; Mobile Phase A: Water (10 mmol/L NH₄HCO₃+0.1% NH₃·H₂O), Mobile Phase B: ACN; Flow rate: 60 mL/min; Gradient: 30 B to 40 B in 7 min; Wave Length: 254/220 nm to afford the title compound (20 mg, 12.9%) as a white solid. LC-MS: (ES, m/z): RT=1.287 min, LCMS: m/z=538 [M+1]. ¹H NMR (300 MHz, DMSO-d₆) δ 10.28 (s, 1H), 9.33 (s, 1H), 8.99-8.88 (m, 2H), 8.56 (s, 1H), 8.12 (d, J=8.8 Hz, 1H), 7.93-7.84 (m, 1H), 7.75 (dd, J=8.9, 2.2 Hz, 1H), 7.70-7.46 (m, 2H), 4.83 (s, 1H), 3.67 (s, 3H), 2.70 (s, 2H), 2.24 (d, J=24.4 Hz, 7H), 1.99 (d, J=10.2 Hz, 2H),

### Example A18: N-(7-(3-(difluoromethoxy)-1-methyl-1H-pyrazol-4-yl)-5-((1-methylpiperidin-4-yl)oxy)quinazolin-4-yl)benzo[d]thiazol-6-amine

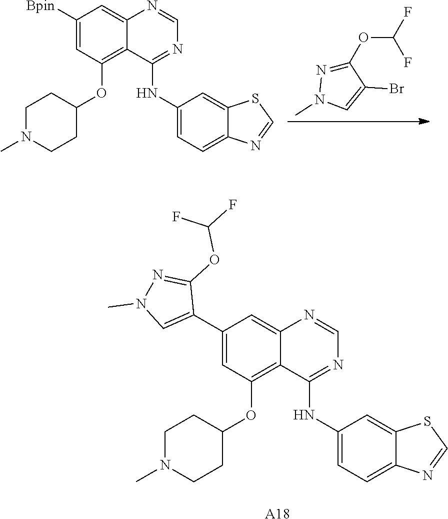

To a solution of Intermediate BP-54 (87.7 mg, 0.3865 mmol, 1.00 eq) in 1,4-dioxane/H₂O was added Intermediate BP-73 (200 mg, 0.3865 mmol, 1.00 eq), K₂CO₃ (106 mg, 0.77 mmol, 2.00 eq), Pd(dppf)Cl₂ (12.5 mg, 0.02 mmol, 0.05 eq) under nitrogen. The mixture was stirred at 80° C. for 3 hr. The reaction mixture was cooled to room temperature, diluted with 20 mL of water, extracted with 2×20 mL of EA, the organic layers combined and washed with 20 mL of brine. The solution was dried over anhydrous sodium sulfate and concentrated under vacuum. The crude product was purified by Prep-HPLC: Column: XBridge Shield RP18 OBD Column, 30*150 mm, 5 um; Mobile Phase A: Water (0.05% NH₃H₂O), Mobile Phase B: ACN; Flow rate: 60 mL/min; Gradient: 43 B to 51 B in 8 min; Wave Length: 254/220 nm to afford the title compound (20 mg, 12.9%) as a white solid. LC-MS: (ES, m/z): RT=2.195 min, LCMS: m/z=538 [M+1]. ¹H NMR (300 MHz, DMSO-d₆) δ 10.24 (s, 1H), 9.31 (s, 1H), 8.89 (d, J=2.2 Hz, 1H), 8.52 (d, J=23.7 Hz, 2H), 8.10 (d, J=8.8 Hz, 1H), 7.78-7.66 (m, 1H), 7.60-7.16 (m, 3H), 4.85 (s, 1H), 3.82 (s, 3H), 2.79 (s, 2H), 2.24 (s, 7H), 2.01 (s, 2H),

### Example A19: (R)-1-(1-(4-((7-fluorobenzo[d]thiazol-6-yl)amino)-5-(((R)-1-methoxypropan-2-yl)oxy)quinazolin-7-yl)-1H-pyrazol-4-yl)ethan-1-ol OR (S)-1-(1-(4-((7-fluorobenzo[d]thiazol-6-yl)amino)-5-(((R)-1-methoxypropan-2-yl)oxy)quinazolin-7-yl)-1H-pyrazol-4-yl)ethan-1-ol

### Example A20: (S)-1-(1-(4-((7-fluorobenzo[d]thiazol-6-yl)amino)-5-(((R)-1-methoxypropan-2-yl)oxy)quinazolin-7-yl)-1H-pyrazol-4-yl)ethan-1-ol, OR (R)-1-(1-(4-((7-fluorobenzo[d]thiazol-6-yl)amino)-5-(((R)-1-methoxypropan-2-yl)oxy)quinazolin-7-yl)-1H-pyrazol-4-yl)ethan-1-ol

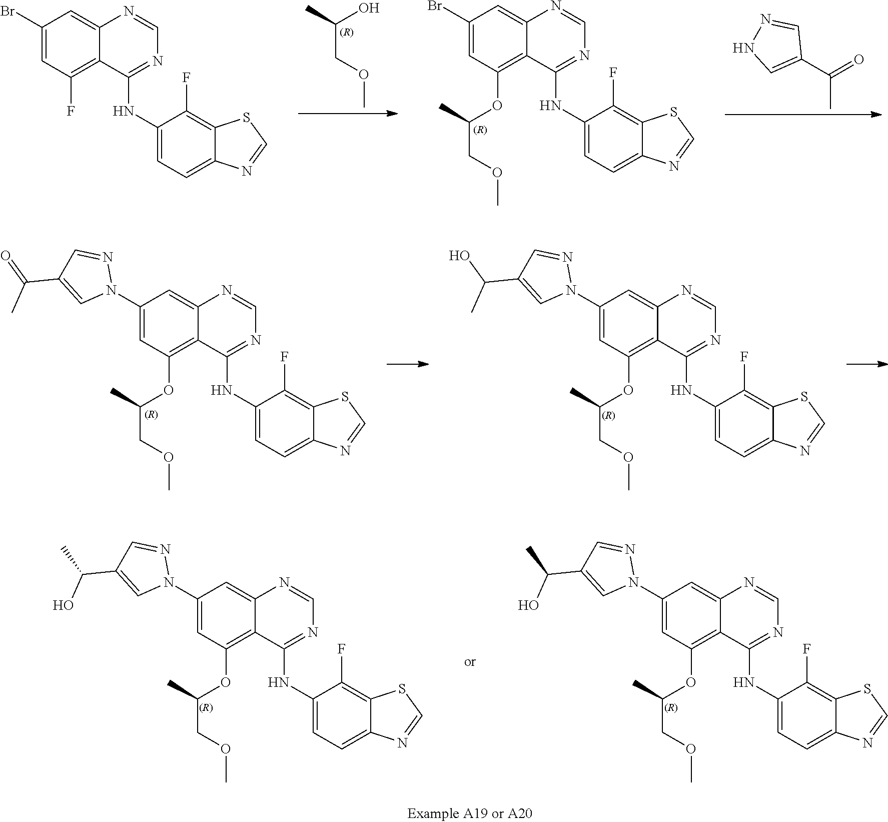

### Step 1: (R)—N-(7-bromo-5-((1-methoxypropan-2-yl)oxy)quinazolin-4-yl)-7-fluorobenzo[d]thiazol-6-amine

A mixture Intermediate BP-60 (200 mg, 508 μmol), (2R)-1-methoxypropan-2-ol (136 mg, 1.52 mmol), tBuOK (113 mg, 1.01 mmol) in THF (15 mL) was stirred at 80° C. for 16 hours. Concentrated to dryness. The residue was purified on prep-TLC with EA:PE=1:1 to afford the title compound (95 mg, yield: 40%) as an off-white solid. LC-MS: (ES, m/z): RT=1.328 min, LCMS: m/z=463,465 [M+1]

### Step 2: (R)—N-(7-bromo-5-((1-methoxypropan-2-yl)oxy)quinazolin-4-yl)-7-fluorobenzo[d]thiazol-6-amine

A mixture of (R)—N-(7-bromo-5-((1-methoxypropan-2-yl)oxy)quinazolin-4-yl)-7-fluorobenzo[d]thiazol-6-amine (70 mg, 151 μmol), 1-(1H-pyrazol-4-yl)ethan-1-one (49.8 mg, 453 μmol), EPhos Pd G4 (13.8 mg, 15.1 μmol), Cs₂CO₃ (73.6 mg, 226 μmol) in Dioxane (10 mL) was stirred at 100° C. for 4 hr. Concentrated to dryness. The residue was purified on prep-TLC with DCM:MeOH=25:1 to afford the title compound (50 mg, yield: 67%) as a yellow solid. LC-MS: (ES, m/z): RT=0.832 min, LCMS: m/z=493 [M+1]

### Step 3: 1-(1-(4-((7-fluorobenzo[d]thiazol-6-yl)amino)-5-(((R)-1-methoxypropan-2-yl)oxy)quinazolin-7-yl)-1H-pyrazol-4-yl)ethan-1-ol

To a solution of (R)—N-(7-bromo-5-((1-methoxypropan-2-yl)oxy)quinazolin-4-yl)-7-fluorobenzo[d]thiazol-6-amine (80 mg, 162 μmol) in MeOH (10 mL) was added NaBH₄ (18.3 mg, 485 μmol). Stirred at 25° C. for 1 hour. Quenched with water. Concentrated to dryness. The residue was purified on prep-HPLC to afford the a racemic mixture.

### Step 4: Chiral Separation

The racemates was separated by prep-Chiral-HPLC, Column: CHIRALPAK IE, 2*25 cm, 5 μm; Mobile Phase A: MTBE (0.5% 2M NH₃-MeOH), Mobile Phase B: IPA; Flow rate: 16 mL/min; Gradient: 40% B to 40% B in 23 min; Wave Length: 220/254 nm; RT1 (min): 13.865; RT2 (min): 18.617.

Example A19: First Eluting Compound: (R)-1-(1-(4-((7-fluorobenzo[d]thiazol-6-yl)amino)-5-(((R)-1-methoxypropan-2-yl)oxy)quinazolin-7-yl)-1H-pyrazol-4-yl)ethan-1-ol OR (S)-1-(1-(4-((7-fluorobenzo[d]thiazol-6-yl)amino)-5-(((R)-1-methoxypropan-2-yl)oxy)quinazolin-7-yl)-1H-pyrazol-4-yl)ethan-1-ol. (5.1 mg) as an off-white solid. LC-MS: (ES, m/z): RT=1.071 min, LCMS: m/z=495 [M+1]. CHIRAL-HPLC: RT=2.610 min. ¹H NMR (400 MHz, Chloroform-d) δ 10.37 (s, 1H), 8.96 (d, J=1.5 Hz, 1H), 8.70-8.61 (m, 2H), 8.09 (s, 1H), 8.01 (d, J=8.9 Hz, 1H), 7.77 (s, 1H), 7.71 (d, J=2.0 Hz, 1H), 7.60 (d, J=2.0 Hz, 1H), 5.11-4.98 (m, 1H), 3.76 (d, J=4.5 Hz, 2H), 3.38 (s, 3H), 1.60 (dd, J=6.4, 3.8 Hz, 6H),

Example A20: Second Eluting Compound (S)-1-(1-(4-((7-fluorobenzo[d]thiazol-6-yl)amino)-5-(((R)-1-methoxypropan-2-yl)oxy)quinazolin-7-yl)-1H-pyrazol-4-yl)ethan-1-ol, OR (R)-1-(1-(4-((7-fluorobenzo[d]thiazol-6-yl)amino)-5-(((R)-1-methoxypropan-2-yl)oxy)quinazolin-7-yl)-1H-pyrazol-4-yl)ethan-1-ol (4.5 mg) as an off-white solid. LC-MS: (ES, m/z): RT=1.069 min, LCMS: m/z=495 [M+1], CHIRAL-HPLC: RT=3.550 min, ¹H NMR (400 MHz, Chloroform-d) δ 10.37 (s, 1H), 8.96 (d, J=1.5 Hz, 1H), 8.70-8.61 (m, 2H), 8.09 (s, 1H), 8.01 (d, J=8.9 Hz, 1H), 7.77 (s, 1H), 7.71 (d, J=2.0 Hz, 1H), 7.60 (d, J=2.0 Hz, 1H), 5.11-4.98 (m, 1H), 3.76 (d, J=4.5 Hz, 2H), 3.38 (s, 3H), 1.60 (dd, J=6.4, 3.8 Hz, 6H).

### Example A22: (1R,2S,4s)-4-((4-(benzo[d]thiazol-6-ylamino)-7-(1-methyl-1H-pyrazol-4-yl)quinazolin-5-yl)oxy)cyclopentane-1,2-diol OR (1R,2S,4r)-4-((4-(benzo[d]thiazol-6-ylamino)-7-(1-methyl-1H-pyrazol-4-yl)quinazolin-5-yl)oxy)cyclopentane-1,2-diol

### Example A23: (1R,2S,4r)-4-((4-(benzo[d]thiazol-6-ylamino)-7-(1-methyl-1H-pyrazol-4-yl)quinazolin-5-yl)oxy)cyclopentane-1,2-diol OR (1R,2S,4s)-4-((4-(benzo[d]thiazol-6-ylamino)-7-(1-methyl-1H-pyrazol-4-yl)quinazolin-5-yl)oxy)cyclopentane-1,2-diol

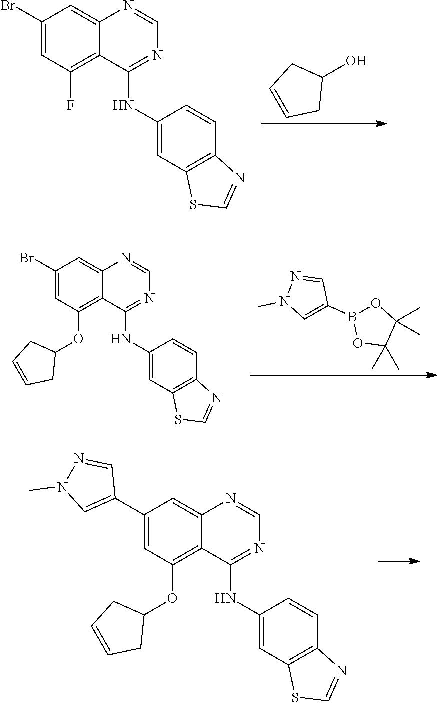

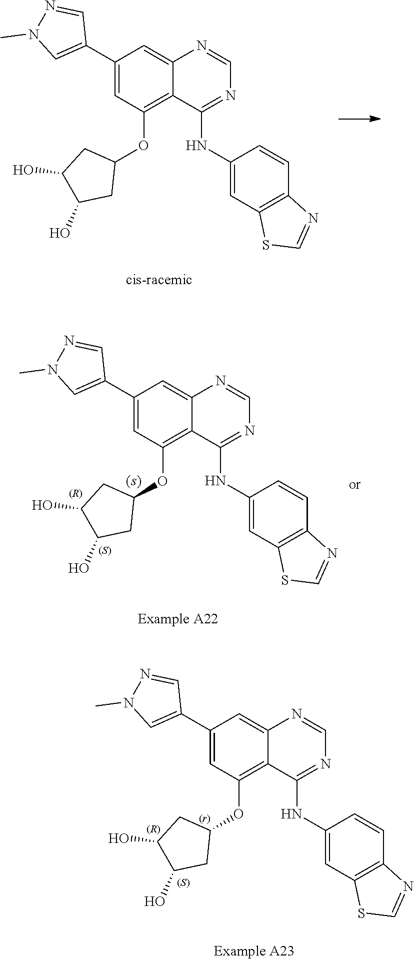

### Step 1: N-(7-bromo-5-(cyclopent-3-en-1-yloxy)quinazolin-4-yl)benzo[d]thiazol-6-amine

To the solution of cyclopent-3-en-1-ol (201 mg, 2.39 mmol) in DMF (10 ml) at 0C was added NaH (63.5 mg, 1.59 mmol). The mixture was stirred at 0° C. for 0.5 h. Intermediate BP-59 (300 mg, 799 umol) was added, stirred at 100° C. for 12 h. The reaction mixture was diluted with water (50 mL), and extracted with EA (80 mL×3), the organic layers washed saturated brine (10 mL×1), and evaporated. The crude product was purified by column chromatography (DCM:MeOH=20:1) to afford the title compound (210 mg, 59.8%) as a yellow solid. LC-MS: (ES, nm/z): RT=1.886 min, LCMS: nm/z=439 [M+1].

### Step 2: N-(5-(cyclopent-3-en-1-yloxy)-7-(1-methyl-1H-pyrazol-4-yl)quinazolin-4-yl)benzo[d]thiazol-6-amine

In a 8 ml sealed tube, N-(7-bromo-5-(cyclopent-3-en-1-yloxy)quinazolin-4-yl)benzo[d]thiazol-6-amine (210 mug, 478 umol), 1-methyl-4-(4,4,5,5-tetramethyl-1,3,2-dioxaborolan-2-yl)-1H-pyrazole (119 mg, 573 umol), K₂CO₃ (131 mug, 956 umol), Pd(dppf)Cl₂ (34.9 mg, 47.8 umol) in dioxane (3 ml) and H2O (1 ml) were added and heated to 80° C. for 3 h under nitrogen. The reaction mixture was diluted with water (100 mL), extracted with EA (100 mL×3) and washed with saturated brine (100 mL×1). The organic layer was dried over Na₂SO₄, and concentrated. Purified by column chromatography (DCM:MeOH=15:1) to afford the title compound (190 mg, 90.4%) as a yellow solid. LC-MS: (ES, nm/z): RT=1.559 min LCMS: nm/z=441 [M+1].

### Step 3: 4-((4-(benzo[d]thiazol-6-ylamino)-7-(1-methyl-1H-pyrazol-4-yl)quinazolin-5-yl)oxy)cyclopentane-1,2-diol

To a solution of N-(5-(cyclopent-3-en-1-yloxy)-7-(1-methyl-1H-pyrazol-4-yl)quinazolin-4-yl)benzo[d]thiazol-6-amine (190 mg, 431 umol) in THF (3 ml) and H₂O (1 ml) was added NMO (150 mg, 1.29 mmol) and K₂OsO₄ (2.86 mg, 8.62 umol). Stirred at rt for 12 h. The reaction mixture was filtered the solid was washed by THF and H2O. The filtrate was evaporated to afford the title compound (85 mg, 41.6%) as a grey solid. LC-MS: (ES, m/z): RT=1.210 min, LCMS: m/z=475 [M+1].

### Step 4: Chiral Separation

The crude product 4-((4-(benzo[d]thiazol-6-ylamino)-7-(1-methyl-1H-pyrazol-4-yl)quinazolin-5-yl)oxy)cyclopentane-1,2-diol (80 mg, 168 μmol) was purified by preparative Chiral HPLC (Column: CHIRALPAK IA, 2*25 cm, 5 μm; Mobile Phase A: Hex (0.5% 2M NH3-MeOH)-HPLC, Mobile Phase B: EtOH-HPLC; Flow rate: 18 mL/min; Gradient: 50% B to 50% B in 12.5 min; Wave Length: 220/254 nm; RT1 (min): 7.628; RT2 (min): 10.897; Sample Solvent: MeOH:DCM=1:1; Injection Volume: 1 mL; to afford:

Example A22: First Eluting Peak (1R,2S,4s)-4-((4-(benzo[d]thiazol-6-ylamino)-7-(1-methyl-1H-pyrazol-4-yl)quinazolin-5-yl)oxy)cyclopentane-1,2-diol OR (1R,2S,4r)-4-((4-(benzo[d]thiazol-6-ylamino)-7-(1-methyl-1H-pyrazol-4-yl)quinazolin-5-yl)oxy)cyclopentane-1,2-diol (23 mg, 28.8%), as a light yellow solid. LC-MS: (ES, m/z): RT=1.089 min, LCMS: m/z=475 [M+1], Chiral-HPLC (ES): RT=1.495, ¹H NMR (300 MHz, DMSO-d6) δ 10.49 (s, 1H), 9.31 (s, 1H), 8.87 (d, J=2.0 Hz, 1H), 8.52 (s, 1H), 8.46 (s, 1H), 8.14 (s, 1H), 8.08-7.99 (m, 2H), 7.53 (d, J=1.5 Hz, 1H), 7.23 (d, J=1.6 Hz, 1H), 5.28 (d, J=7.3 Hz, 1H), 4.96 (d, J=4.1 Hz, 2H), 4.03 (d, J=4.1 Hz, 2H), 3.92 (s, 3H), 2.48-2.42 (m, 2H), 1.98 (d, J=14.6 Hz, 2H),

Example A23: (1R,2S,4r)-4-((4-(benzo[d]thiazol-6-ylamino)-7-(1-methyl-1H-pyrazol-4-yl)quinazolin-5-yl)oxy)cyclopentane-1,2-diol OR (1R,2S,4s)-4-((4-(benzo[d]thiazol-6-ylamino)-7-(1-methyl-1H-pyrazol-4-yl)quinazolin-5-yl)oxy)cyclopentane-1,2-diol. (6 mg, 7.52%) as a light yellow solid. LC-MS: (ES, m/z): RT=1.081 min, LCMS: m/z=475 [M+1], Chiral-HPLC (ES): RT=2.279, ¹H NMR (300 MHz, DMSO-d6) δ 10.21 (s, 1H), 9.32 (s, 1H), 8.93 (d, J=2.1 Hz, 1H), 8.57 (s, 1H), 8.50 (s, 1H), 8.17-8.10 (m, 2H), 7.73 (dd, J=8.9, 2.2 Hz, 1H), 7.56 (d, J=1.5 Hz, 1H), 7.23 (d, J=1.5 Hz, 1H), 5.39 (s, 1H), 4.70 (d, J=3.9 Hz, 2H), 4.19 (s, 2H), 3.92 (s, 3H), 2.39 (dt, J=13.3, 6.3 Hz, 2H), 2.15 (d, J=14.5 Hz, 2H),

### Example A24: 7-fluoro-N-(7-(3-(4-methylpiperazin-1-yl)prop-1-yn-1-yl)-5-((tetrahydro-2H-pyran-4-yl)oxy)quinazolin-4-yl)benzo[d]thiazol-6-amine

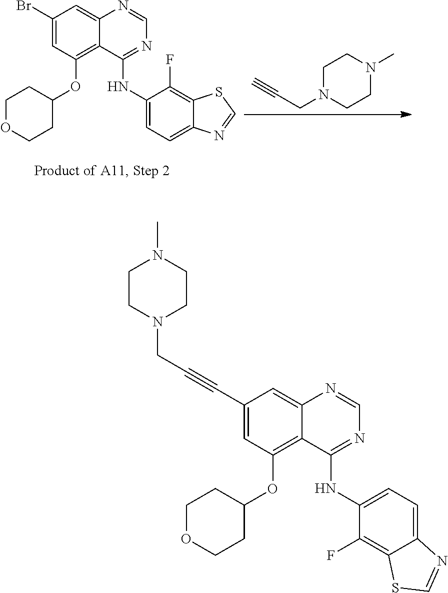

To a reaction vessel was added: TEA (168 mg, 1.67 mmol) was added to Pd(dppf)Cl₂·THF (12.8 mg, 16.8 μmol), CuI (6.45 mg, 33.6 μmol), N-(7-bromo-5-((tetrahydro-2H-pyran-4-yl)oxy)quinazolin-4-yl)-7-fluorobenzo[d]thiazol-6-amine (product of A11, Step 2, 80 mg, 168 μmol), 1-methyl-4-(prop-2-yn-1-yl)piperazine (69.6 mg, 504 μmol) and DMF at rt. The reaction was heated to 100° C. for 16 h. The reaction was diluted with EA (50 mL) and washed with brine (50 mL*2). The organic layer was dried with Na₂SO₄ and concentrated under vacuum. The residue was purified by prep-HPLC: Column: XBridge Prep OBD C18 Column, 30×150 mm Sum; Mobile Phase A: Water (10 mmol/L NH₄HCO₃+0.1% NH₃·H₂O), Mobile Phase B: ACN; Flow rate: 60 mL/min; Gradient: 27 B 10 to 51 B in 7 min; 254/220 nm to afford the title compound (20 mg) as a white solid. LC-MS: (ES, m/z): RT=1.999 min, m/z=533 [M+1], ¹H NMR (300 MHz, DMSO-d6) δ 10.21 (s, 1H), 9.44 (d, J=1.6 Hz, 1H), 8.76-8.64 (m, 1H), 8.60 (s, 1H), 8.04 (d, J=8.9 Hz, 1H), 7.43 (d, J=1.3 Hz, 1H), 7.35 (s, 1H), 5.15 (s, 1H), 3.95 (d, J=11.9 Hz, 2H), 3.58-3.50 (m, 4H), 2.58-2.55 (s, 3H), 2.55-2.38 (m, 4H), 2.18 (s, 6H), 1.90-1.85 (m, 2H),

### Example A25: N-(1,3-benzothiazol-6-yl)-5-{[(3S)-1-methylpiperidin-3-yl]oxy}-7-[3-(morpholin-4-yl)prop-1-yn-1-yl]quinazolin-4-amine

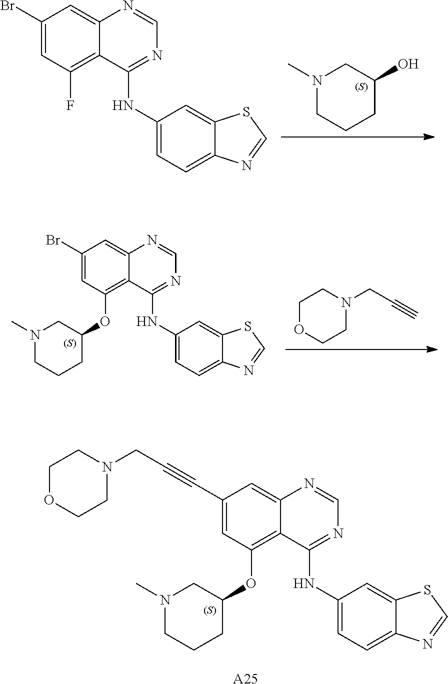

### Step 1: (S)—N-(7-bromo-5-((1-methylpiperidin-3-yl)oxy)quinazolin-4-yl)benzo[d]thiazol-6-amine

t-BuOK (118 mg, 1.06 mmol) was added to Intermediate BP-59 (200 mg, 533 μmol) and (3S)-1-methylpiperidin-3-ol (122 mg, 1.06 mmol) in THF (5 mL) at rt. The reaction mixture was heated to 80° C. for 2 h. The reaction mixture was diluted with water, and extracted with EA and saturated brine. The organic layer was dried over Na₂SO₄, filtered, evaporated, and purified by column chromatography (DCM:MeOH=25:1) to afford the title compound (200 mg, 79.8%) as a yellow solid. LC-MS: (ES, m/z): RT=0.615 min, LCMS: m/z=470[M+1].

### Step 2: (S)—N-(5-((1-methylpiperidin-3-yl)oxy)-7-(3-morpholinoprop-1-yn-1-yl)quinazolin-4-yl)benzo[d]thiazol-6-amine

To a reaction vessel under nitrogen was added: (S)—N-(7-bromo-5-((1-methylpiperidin-3-yl)oxy)quinazolin-4-yl)benzo[d]thiazol-6-amine (100 mg, 212 umol), 4-(prop-2-yn-1-yl)morpholine (53.0 mg, 424 umol), Pd(dppf)Cl₂ (15.4 mg, 21.2 umol), TEA (213 mg, 2.11 mmol), CuI (8.05 mg, 42.4 umol) and DMF (3 ml). The reaction was heated at 100° C. for 12 h. The reaction mixture was diluted with water, extracted with EA and washed with saturated brine. The organic layer was dried over Na₂SO₄, filtered, evaporated, and purified by column chromatography (DCM:MeOH=15:1) then further purified by Prep-HPLC (Column: XBridge Prep OBD C18 Column, 30*150 mm, 5 μm; Mobile Phase A: Water (10 MMOL/L NH₄HCO₃+0.1% NH₃·H₂O), Mobile Phase B: ACN; Flow rate: 60 mL/min; Gradient: 44% B to 59% B in 7 min; Wave Length: 254/220 nm, to afford the title compound (17.7 mg, 15.3%) as a white solid. LC-MS: (ES, m/z): RT=1.264 min, LCMS: m/z=515 [M+1], ¹H NMR (400 MHz, DMSO-d6) ¹H δ 10.93 (s, 1H), 9.32 (s, 1H), 8.89 (d, J=2.1 Hz, 1H), 8.60 (s, 1H), 8.12 (d, J=8.8 Hz, 1H), 7.98 (dd, J=8.9, 2.1 Hz, 1H), 7.39 (d, J=1.4 Hz, 1H), 7.21 (d, J=1.5 Hz, 1H), 5.21-5.18 (m, 1H), 3.64 (t, J=4.7 Hz, 4H), 3.59 (s, 2H), 3.16 (d, J=12.2 Hz, 1H), 2.86 (d, J=11.1 Hz, 1H), 2.57 (t, J=4.7 Hz, 4H), 2.30 (s, 4H), 2.07 (t, J=12.0 Hz, 2H), 1.73 (d, J=12.4 Hz, 1H), 1.55 (q, J=13.3, 12.4 Hz, 2H),

### Example A26: (R)-2-(1-(4-((7-fluorobenzo[d]thiazol-6-yl)amino)-5-((1-methoxypropan-2-yl)oxy)quinazolin-7-yl)-1H-pyrazol-4-yl)propan-2-ol

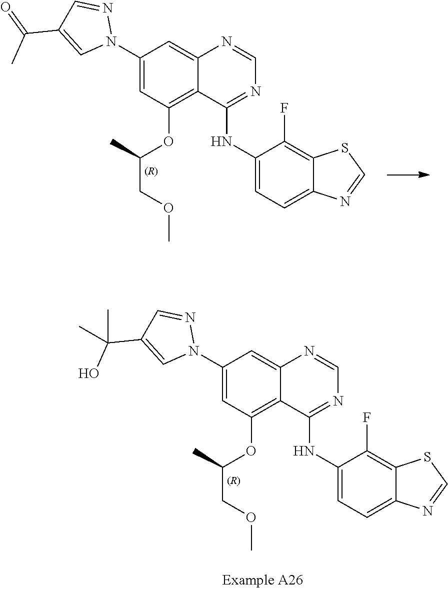

To a solution of (R)-1-(1-(4-((7-fluorobenzo[d]thiazol-6-yl)amino)-5-((1-methoxypropan-2-yl)oxy)quinazolin-7-yl)-1H-pyrazol-4-yl)ethan-1-one (Example A20 step 2, 70 mg, 142 μmol) in THF (10 mL) was added MeMgBr (1 M, 0.7 mL, 709 μmol) at 0° C. Stirred at 25° C. for 1 hour. The reaction was quenched with water and concentrated to dryness. The residue was purified on prep-HPLC, Column: YMC-Actus Triart C18 ExRS, 30 mm×150 mm, Sum; Mobile Phase A: Water (10 mmol/L NH₄HCO₃+0.1% NH₃·H₂O), Mobile Phase B: ACN; Flow rate: 60 mL/min; Gradient: 30% B to 64% B in 7 min; 254/220 nm; RT1: 6.92 min, to afford the title compound (13.4 mg) as a white solid. LC-MS: (ES, m/z): RT=1.104 min, LCMS: m/z=509 [M+1], ¹H NMR (300 MHz, DMSO-d₆) δ 10.19 (s, 1H), 9.43 (d, J=1.6 Hz, 1H), 8.66 (s, 1H), 8.52 (s, 1H), 8.48-8.36 (m, 1H), 8.02 (d, J=8.9 Hz, 1H), 7.82-7.76 (m, 2H), 7.73 (s, 1H), 5.26 (s, 1H), 5.02 (s, 1H), 3.77-3.68 (m, 2H), 1.47 (d, J=7.2 Hz, 9H),

### Example A27: (S)-7-fluoro-N-(6-fluoro-7-(1-methyl-1H-pyrazol-4-yl)-5-(1-(oxetan-3-yl)ethoxy)quinazolin-4-yl)benzo[d]thiazol-6-amine OR (R)-7-fluoro-N-(6-fluoro-7-(1-methyl-1H-pyrazol-4-yl)-5-(1-(oxetan-3-yl)ethoxy)quinazolin-4-yl)benzo[d]thiazol-6-amine

### Example A28: (R)-7-fluoro-N-(6-fluoro-7-(i-methyl-1H-pyrazol-4-yl)-5-(1-(oxetan-3-yl)ethoxy)quinazolin-4-yl)benzo[d]thiazol-6-amine OR (S)-7-fluoro-N-(6-fluoro-7-(1-methyl-1H-pyrazol-4-yl)-5-(1-(oxetan-3-yl)ethoxy)quinazolin-4-yl)benzo[d]thiazol-6-amine

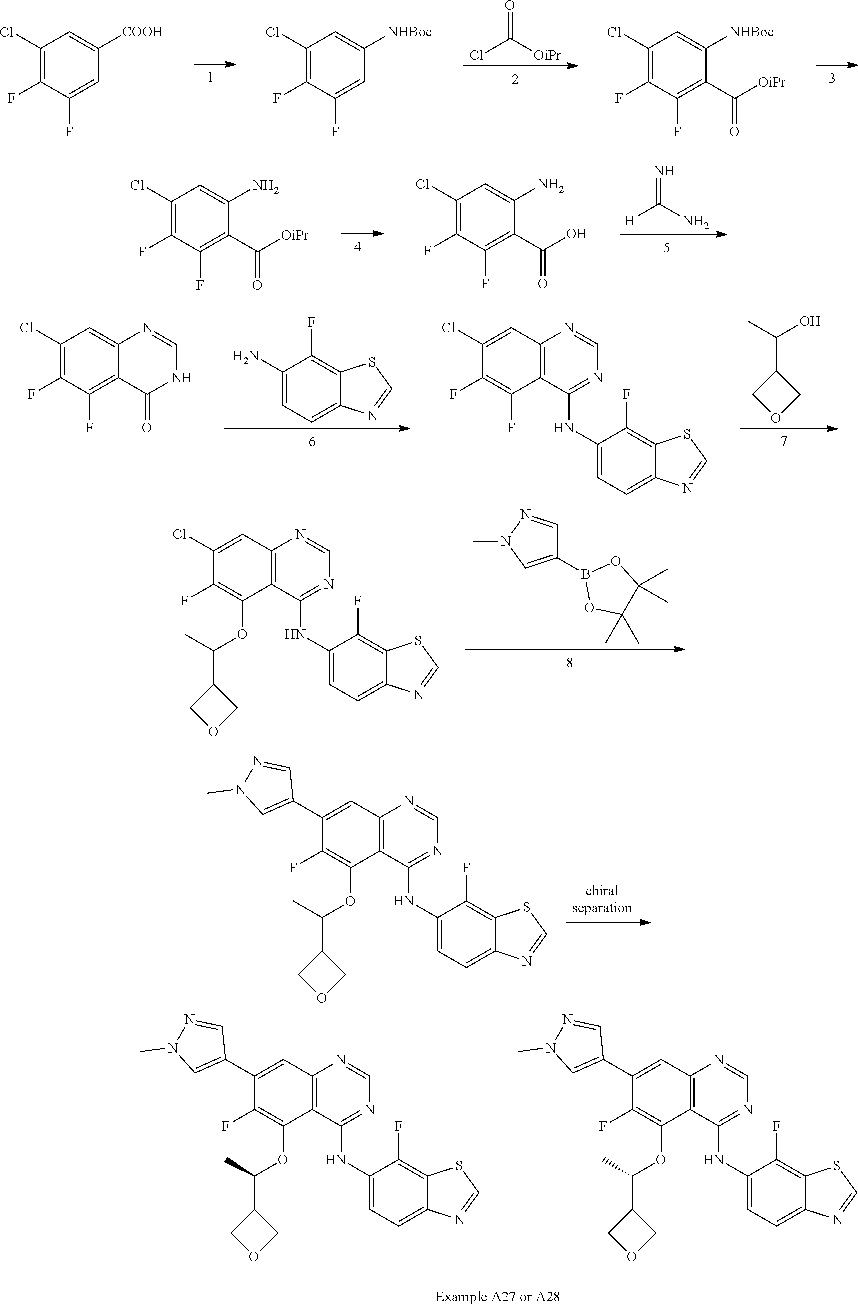

### Step 1: tert-butyl N-(3-chloro-4,5-difluorophenyl)carbamate

TEA (1.56 g, 15.5 mmol) was added to the reaction mixture of 3-chloro-4,5-difluorobenzoic acid (1 g, 5.19 mmol), DPPA (2.83 g) in t-BuOH (5 mL) at 0° C. under N2, then heated to reflux and stirred for 10 h. After completion of the reaction, concentrated and purified by gel silica chromatography with PE/EA=1:1. The resulted in 1 g tert-butyl 3-chloro-4,5-difluorophenylcarbamate as a white solid. LC-MS: (ES, m/z): RT=0.834 min, LCMS: m/z=262 [M−1]

### Step 2: tert-butyl (3-chloro-4,5-difluorophenyl)carbamate

s-BuLi (2.96 mL, 2.96 mmol) in THF was added to tert-butyl N-(3-chloro-4,5-difluorophenyl)carbamate (523 mg, 1.98 mmol) in THF (8 mL) at −78° C. under N₂, and stirred for 30 min at this temperature. Then propan-2-yl carbonochloridate (362 mg, 2.96 mmol) in THF (2 mL) was added the mixture at −78° C., then stirred at rt for 10 h. After quenched with saturated NH₄C1, concentrated. The residue was purified by prep-TLC with PE:EA=2:1. The resulted in 400 mg isopropyl 6-(tert-butoxycarbonylamino)-4-chloro-2,3-difluorobenzoate as an off-white solid. LC-MS: (ES, m/z): RT=0.920 min, LCMS: m/z=347 [M−1]

### Step 3: isopropyl 6-amino-4-chloro-2,3-difluorobenzoate

HCl in dioxane (1 mL, 4M) was added to a solution of tert-butyl (3-chloro-4,5-difluorophenyl)carbamate (280 mg) in EA (1 mL) was stirred for 2 h. The solid was collected by filtration. The resulted in 200 mg propan-2-yl 6-amino-4-chloro-2,3-difluorobenzoate as a yellow solid. LC-MS: (ES, m/z): RT=0.807 min, LCMS: m/z=247 [M−1]

### Step 4: 6-amino-4-chloro-2,3-difluorobenzoic acid

LiOH (23.9 mg, 1 mmol) was added isopropyl 6-amino-4-chloro-2,3-difluorobenzoate (247 mg, 0.1 mmol) THF (2 mL), and H2O (0.5 mL) was followed, then stirred for 10 h. After completion of the reaction, the reaction acidified by HCl (1 M), then extracted with DCM, and dried by Na₂SO₄, and concentrate. The resulted in 200 mg 6-amino-4-chloro-2,3-difluorobenzoic acid as a white solid. LC-MS: (ES, m/z): RT=0.366 min, LCMS: m/z=208 [M+1]206[M−1]

### Step 5: 7-chloro-5,6-difluoroquinazolin-4(3H)-one

6-amino-4-chloro-2,3-difluorobenzoic acid (207 mg, 1 mmol) and methanimidamide AcOH (517 mg, 5 mmol) was dissolved into EtOH (5 mL), and heated to reflux, and stirred for 10 h. After completion of the reaction. The reaction solution was cooled down to rt, and the product was collected by filter. The resulted in 200 mg 7-chloro-5,6-difluoro-3,4-dihydroquinazolin-4-one as a white solid. LC-MS: (ES, m/z): RT=0.514 min, LCMS: m/z=214.9 [M−1]

### Step 6: N-(7-chloro-5,6-difluoroquinazolin-4-yl)-7-fluorobenzo[d]thiazol-6-amine

PPh₃ (1.92 g, 7.32 mmol) and CCl₄ (2.23 g, 14.6 mmol) were added to 7-chloro-5,6-difluoroquinazolin-4(3H)-one (530 mg, 2.44 mmol) in DCE (20 mL) at rt. The mixture was stirred at 80° C. for 2 h. The reaction mixture was cooled to rt. Then a solution of 7-fluoro-1,3-benzothiazol-6-amine (1.23 g, 7.32 mmol) was added, the reaction mixture was heated to 50° C. for 1 h. The mixture was filtered and the filter cake was collected. The resulted in 660 mg, 73%) 7-chloro-5,6-difluoro-N-(7-fluoro-1,3-benzothiazol-6-yl)quinazolin-4-amine as a yellow solid. LC-MS: (ES, m/z): RT=1.034 min, LCMS: m/z=367 [M+1]

### Step 7: N-(7-chloro-6-fluoro-5-(1-(oxetan-3-yl)ethoxy)quinazolin-4-yl)-7-fluorobenzo[d]thiazol-6-amine

t-BuOK (400 mg, 3.58 mmol) was added to N-(7-chloro-5,6-difluoroquinazolin-4-yl)-7-fluorobenzo[d]thiazol-6-amine (660 mg, 1.79 mmol) and (1R)-1-(oxetan-3-yl)ethan-1-ol (365 mg, 3.58 mmol) in THF (10 mL) at rt. The reaction mixture was heated to 80° C. for 2 h. The resulting solution was extracted with 3×50 mL of EA. The organic layer was dried with Na₂SO₄ and concentrated under vacuum. The crude product was purified by prep-TLC with DCM:MeOH=25:1. The resulted in 300 mg, 37%) N-(7-chloro-6-fluoro-5-(1-(oxetan-3-yl)ethoxy)quinazolin-4-yl)-7-fluorobenzo[d]thiazol-6-amine as a yellow solid. LC-MS: (ES, m/z): RT=0.867 min, LCMS: m/z=448 [M+1]

### Step 8: 7-fluoro-N-(6-fluoro-7-(1-methyl-1H-pyrazol-4-yl)-5-(1-(oxetan-3-yl)ethoxy)quinazolin-4-yl)benzo[d]thiazol-6-amine

Pd(dppf)Cl₂ (25.6 mg, 33.4 umol) and K₂CO₃ (69.0 mg, 500 umol) were added to N-(7-chloro-6-fluoro-5-(1-(oxetan-3-yl)ethoxy)quinazolin-4-yl)-7-fluorobenzo[d]thiazol-6-amine (150 mg, 334 μmol) and 1-methyl-4-(4,4,5,5-tetramethyl-1,3,2-dioxaborolan-2-yl)-1H-pyrazole (104 mg, 500 μmol) in H₂O (0.5 mL) and dioxane (2 mL) at rt. The reaction mixture was heated to 80° C. for 2 h under N₂. The resulting solution was extracted with 3×40 mL of EA. The organic layer was dried with Na₂SO₄ and concentrated under vacuum. The crude product was purified by Prep-HPLC as following condition:

Column: XBridge Shield RP18 OBD Column, 30*150 mm, 5 um; Mobile Phase A: Water (10 mmol/L NH₄HCO₃+0.1% NH₃·H₂O), Mobile Phase B: ACN; Flow rate: 60 mL/min; Gradient: 37 B to 47 B in 8 min; Wave Length: 254/220 nm; The resulted in 50 mg, 30%) 6-fluoro-N-(7-fluoro-1,3-benzothiazol-6-yl)-7-(1-methyl-1H-pyrazol-4-yl)-5-[1-(oxetan-3-yl)ethoxy]quinazolin-4-amine as a white solid. LC-MS: (ES, m/z): RT=0.592 min, LCMS: m/z=495 [M+1]

### Step 9: Chiral Separation

6-fluoro-N-(7-fluoro-1,3-benzothiazol-6-yl)-7-(1-methyl-1H-pyrazol-4-yl)-5-[1-(oxetan-3-yl)ethoxy]quinazolin-4-amine (50 mg, 101 μmol) in MeOH was purified by Prep-Chiral-HPLC as following condition: Column: CHIRAL ART Cellulose-SB, 2*25 cm, 5 μm; Mobile Phase A: MTBE (0.5% 2M NH₃-MeOH)-HPLC, Mobile Phase B: EtOH-HPLC; Flow rate: 20 mL/min; Gradient: 10% B to 10% B in 15 min; Wave Length: 220/254 nm; RT1 (min): 11.1; RT2 (min): 12.8; Sample Solvent: MTBE (0.5% 2M NH₃-MeOH)-HPLC; to afford:

Example A27, First eluting compound: (S)-7-fluoro-N-(6-fluoro-7-(1-methyl-1H-pyrazol-4-yl)-5-(1-(oxetan-3-yl)ethoxy)quinazolin-4-yl)benzo[d]thiazol-6-amine OR (R)-7-fluoro-N-(6-fluoro-7-(1-methyl-1H-pyrazol-4-yl)-5-(1-(oxetan-3-yl)ethoxy)quinazolin-4-yl)benzo[d]thiazol-6-amine (15.9 mg, 31%) as an off-white solid. LC-MS: (ES, m/z): RT=0.864 min, LCMS: m/z=495 [M+1], chiral-HPLC: (ES, m/z): R=4.634 min, ¹H NMR (400 MHz, DMSO-d₆) δ 9.86-9.81 (m, 1H), 9.45 (d, J=1.5 Hz, 1H), 8.56-8.47 (m, 2H), 8.44 (d, J=2.7 Hz, 1H), 8.17 (d, J=1.7 Hz, 1H), 8.03 (d, J=8.9 Hz, 1H), 7.94 (d, J=6.8 Hz, 1H), 5.17-5.05 (m, 1H), 4.74-4.64 (m, 2H), 4.52 (t, J=6.2 Hz, 1H), 4.45 (t, J=6.2 Hz, 1H), 3.95 (s, 3H), 3.49 (q, J=7.3 Hz, 1H), 1.32 (d, J=6.1 Hz, 3H),

Example A28: Second eluting compound (R)-7-fluoro-N-(6-fluoro-7-(1-methyl-1H-pyrazol-4-yl)-5-(1-(oxetan-3-yl)ethoxy)quinazolin-4-yl)benzo[d]thiazol-6-amine OR (S)-7-fluoro-N-(6-fluoro-7-(1-methyl-1H-pyrazol-4-yl)-5-(1-(oxetan-3-yl)ethoxy)quinazolin-4-yl)benzo[d]thiazol-6-amine, (15.4 mg 30%) as an off-white solid. LC-MS: (ES, m/z): RT=0.854 min, LCMS: m/z=495 [M+1], chiral-HPLC: (ES, m/z): R=5.356 min, 1H NMR (400 MHz, DMSO-d6) δ 9.83 (s, 1H), 9.45 (d, J=1.5 Hz, 1H), 8.56-8.24 (m, 3H), 8.17 (d, J=1.7 Hz, 1H), 8.03 (d, J=8.9 Hz, 1H), 7.95 (d, J=6.8 Hz, 1H), 5.10 (dt, J=12.5, 6.3 Hz, 1H), 4.74-4.64 (m, 2H), 4.52 (t, J=6.2 Hz, 1H), 4.45 (t, J=6.3 Hz, 1H), 3.95 (s, 3H), 3.49 (q, J=7.2 Hz, 1H), 1.32 (d, J=6.1 Hz, 3H),

### Example A29: (3S,4S)-4-((4-(benzo[d]thiazol-6-ylamino)-7-(1-methyl-1H-pyrazol-4-yl)quinazolin-5-yl)oxy)tetrahydrofuran-3-ol, OR (3R,4R)-4-((4-(benzo[d]thiazol-6-ylamino)-7-(1-methyl-1H-pyrazol-4-yl)quinazolin-5-yl)oxy)tetrahydrofuran-3-ol

### Example A30: (3R,4R)-4-((4-(benzo[d]thiazol-6-ylamino)-7-(1-methyl-1H-pyrazol-4-yl)quinazolin-5-yl)oxy)tetrahydrofuran-3-ol OR (3S,4S)-4-((4-(benzo[d]thiazol-6-ylamino)-7-(1-methyl-1H-pyrazol-4-yl)quinazolin-5-yl)oxy)tetrahydrofuran-3-ol

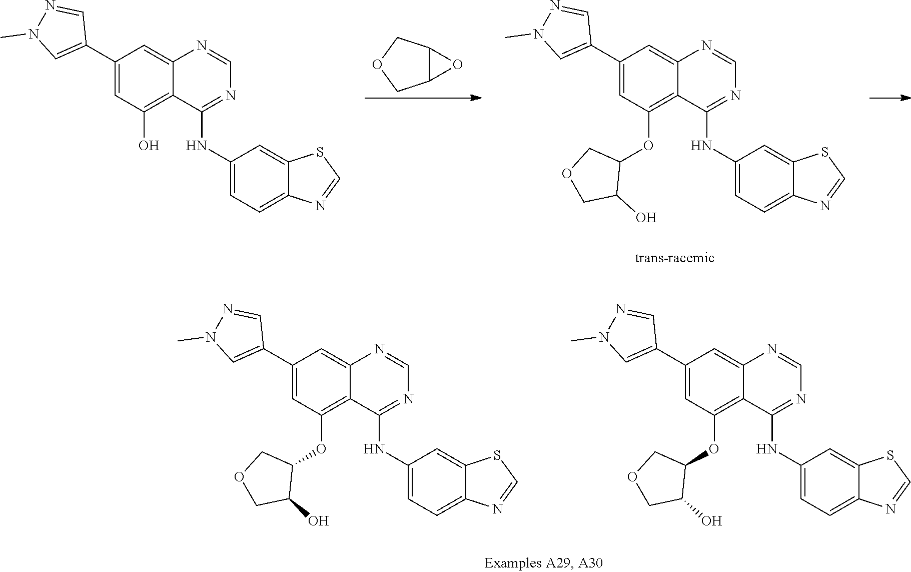 [SMILES: Cn1cc(-c2cc(O)c3c(Nc4ccc5ncsc5c4)ncnc3c2)cn1 ; C1OCC2OC12 ; Cn1cc(-c2cc(OC3COCC3O)c3c(Nc4ccc5ncsc5c4)ncnc3c2)cn1 ; Cn1cc(-c2cc(O[C@H]3COC[C@@H]3O)c3c(Nc4ccc5ncsc5c4)ncnc3c2)cn1 ; Cn1cc(-c2cc(O[C@@H]3COC[C@H]3O)c3c(Nc4ccc5ncsc5c4)ncnc3c2)cn1]

### Step 1: 4-((4-(benzo[d]thiazol-6-ylamino)-7-(1-methyl-1H-pyrazol-4-yl)quinazolin-5-yl)oxy)tetrahydrofuran-3-ol

To Intermediate BP-62 (200 mg, 534 μmol) and 3,6-dioxabicyclo[3.1.0]hexane (137 mg, 1.60 mmol) in 1,4-dioxane (2 mL) was added NaOH (64.0 mg, 1.60 mmol) in H₂O (2 mL) under N₂. The mixture was stirred at 65° C. for 2 days. The reaction was extracted with DCM and purified by Prep-TLC (DCM/MeOH=10:1) to afford the title compound (50 mg, 20%) as yellow solid. LC-MS: (ES, m/z): RT=0.937 min, LCMS: m/z=461 [M+1].

### Step 2: Chiral Separation

4-((4-(benzo[d]thiazol-6-ylamino)-7-(1-methyl-1H-pyrazol-4-yl)quinazolin-5-yl)oxy)tetrahydrofuran-3-ol (10 mg, 21.7 μmol) was purified by Prep-chiral-HPLC: (Column: CHIRALPAK AD-H, 2*25 cm, 5 um; Mobile Phase A: Hex (0.5% 2M NH₃-MeOH)-HPLC, Mobile Phase B: EtOH-HPLC; Flow rate: 18 mL/min; Gradient: 50% B to 50% B in 22 min; 220/254 nm; RTL: 10.789; RT2:19.918) afforded:

Example A29, First Eluting isomer: (3S,4S)-4-((4-(benzo[d]thiazol-6-ylamino)-7-(1-methyl-1H-pyrazol-4-yl)quinazolin-5-yl)oxy)tetrahydrofuran-3-ol, OR (3R,4R)-4-((4-(benzo[d]thiazol-6-ylamino)-7-(1-methyl-1H-pyrazol-4-yl)quinazolin-5-yl)oxy)tetrahydrofuran-3-ol, (4.3 mg, 43%) as a white solid.

LC-MS: (ES, m/z): RT=0.744 min, LCMS: m/z=461 [M+1], Chiral-HPLC (ES): RT=3.941, ¹H NMR (300 MHz, DMSO-d6) δ 10.15 (s, 1H), 9.32 (s, 1H), 9.00 (d, J=2.1 Hz, 1H), 8.60 (s, 1H), 8.46 (s, 1H), 8.17 (s, 1H), 8.11 (d, J=8.8 Hz, 1H), 7.75 (dd, J=8.9, 2.2 Hz, 1H), 7.62 (d, J=1.4 Hz, 1H), 7.48 (s, 1H), 5.81 (d, J=3.7 Hz, 1H), 5.30 (s, 1H), 4.50 (s, 1H), 4.32-4.12 (m, 3H), 3.93 (s, 3H), 3.68 (dd, J=9.8, 2.1 Hz, 1H)

Example A30, Second Eluting isomer: (3R,4R)-4-((4-(benzo[d]thiazol-6-ylamino)-7-(1-methyl-1H-pyrazol-4-yl)quinazolin-5-yl)oxy)tetrahydrofuran-3-ol OR (3S,4S)-4-((4-(benzo[d]thiazol-6-ylamino)-7-(1-methyl-1H-pyrazol-4-yl)quinazolin-5-yl)oxy)tetrahydrofuran-3-ol. (4.6 mg, 46%) as a white solid. LC-MS: (ES, m/z): RT=0.744 min, LCMS: m/z=461 [M+1], Chiral-HPLC (ES): RT=6.713, ¹H NMR (300 MHz, DMSO-d6) δ 10.15 (s, 1H), 9.32 (s, 1H), 9.00 (s, 1H), 8.60 (s, 1H), 8.47 (s, 1H), 8.17 (s, 1H), 8.11 (d, J=8.8 Hz, 1H), 7.76 (d, J=8.1 Hz, 1H), 7.62 (s, 1H), 7.48 (s, 1H), 5.81 (d, J=3.9 Hz, 1H), 5.31 (s, 1H), 4.50 (s, 1H), 4.30-4.11 (m, 3H), 3.93 (s, 3H), 3.68 (d, J=9.5 Hz, 1H),

### Example A31: N-(5-(((3R,4R)-4-methoxytetrahydrofuran-3-yl)oxy)-7-(1-methyl-1H-pyrazol-4-yl)quinazolin-4-yl)benzo[d]thiazol-6-amine OR N-(5-(((3S,4S)-4-methoxytetrahydrofuran-3-yl)oxy)-7-(1-methyl-1H-pyrazol-4-yl)quinazolin-4-yl)benzo[d]thiazol-6-amine

### Example A32: N-(5-(((3S,4S)-4-methoxytetrahydrofuran-3-yl)oxy)-7-(1-methyl-1H-pyrazol-4-yl)quinazolin-4-yl)benzo[d]thiazol-6-amine OR N-(5-(((3R,4R)-4-methoxytetrahydrofuran-3-yl)oxy)-7-(1-methyl-1H-pyrazol-4-yl)quinazolin-4-yl)benzo[d]thiazol-6-amine

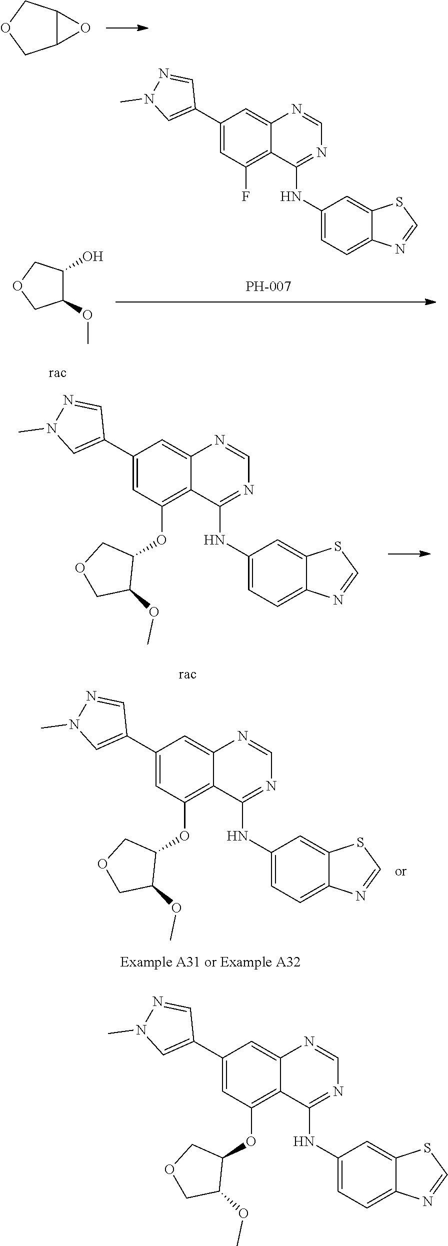

### Step 1: rac-(3S,4S)-4-methoxyoxolan-3-ol

A mixture of 3,6-dioxabicyclo[3.1.0]hexane (500 mg, 5.80 mmol, 1.00 eq) in MeOH (10 mL) was added ceric ammonium nitrate (318 mg, 0.580 mmol, 0.1 eq). The solvent was removed under vacuum. The crude product was purified by column chromatography (EA in PE=0% to 50%), to afford the title compound (300 mg, 51.5%) as a colorless oil.

### Step 2: rac-N-(5-(((3S,4S)-4-methoxytetrahydrofuran-3-yl)oxy)-7-(1-methyl-1H-pyrazol-4-yl)quinazolin-4-yl)benzo[d]thiazol-6-amine

To a mixture of rac-(3S,4S)-4-methoxyoxolan-3-ol (250 mg, 2.12 mmol, 4.00 eq) in DMF (5 mL) was added NaH (38.1 mg, 1.59 mmol, 3.00 eq) at 0° C., the reaction mixture was stirred at 0° C. for 15 min. Intermediate BP-67 (200 mg, 0.5313 mmol, 1.00 eq) was added and the reaction was stirred at 80° C. for 16 hrs. The reaction mixture was added to the ice water. The resulting solution was extracted with 2×30 mL of EA, the organic layers combined and washed with 20 mL of brine. The solution was dried over anhydrous sodium sulfate and concentrated under vacuum. The crude product was purified by column chromatography (EA in PE=0% to 60%), to afford the title compound (120 mg, 47.8%) as a yellow solid. LC-MS: (ES, m/z): RT=1.202 min, LCMS: m/z=475 [M+1].

### Step 3: Chiral Separation

rac-N-(5-(((3S,4S)-4-methoxytetrahydrofuran-3-yl)oxy)-7-(1-methyl-1H-pyrazol-4-yl)quinazolin-4-yl)benzo[d]thiazol-6-amine (120 mg, 0.2528 mmol) was separated by chiral-prep-HPLC: Column: CHIRALPAK IA, 2*25 cm, 5 um; Mobile Phase A: Hex:DCM=3:1 (0.5% 2M NH₃-MeOH)-HPLC, Mobile Phase B: IPA-HPLC; Flow rate: 20 mL/min; Gradient: 15 B to 15 B in 12 min; Wave Length: 220/254 nm to afford:

Example A31, First eluting isomer: N-(5-(((3R,4R)-4-methoxytetrahydrofuran-3-yl)oxy)-7-(1-methyl-1H-pyrazol-4-yl)quinazolin-4-yl)benzo[d]thiazol-6-amine OR N-(5-(((3S,4S)-4-methoxytetrahydrofuran-3-yl)oxy)-7-(1-methyl-1H-pyrazol-4-yl)quinazolin-4-yl)benzo[d]thiazol-6-amine, (38.7 mg). LC-MS: (ES, m/z): RT=1.258 min, LCMS: m/z=475 [M+1]. ¹H NMR (400 MHz, DMSO-d₆) δ 10.07 (s, 1H), 9.32 (s, 1H), 8.98 (d, J=2.2 Hz, 1H), 8.60 (s, 1H), 8.46 (s, 1H), 8.15 (s, 1H), 8.12 (s, OH), 7.79 (dd, J=8.9, 2.2 Hz, 1H), 7.65 (d, J=1.5 Hz, 1H), 7.58 (d, J=1.6 Hz, 1H), 5.63 (d, J=3.3 Hz, 1H), 4.26 (d, J=10.9 Hz, 2H), 4.19 (dd, J=10.1, 5.2 Hz, 1H), 4.09 (dd, J=10.7, 3.4 Hz, 1H), 3.93 (s, 3H), 3.82-3.75 (m, 1H), 3.45 (s, 3H),

Example A32, Second eluting isomer: N-(5-(((3S,4S)-4-methoxytetrahydrofuran-3-yl)oxy)-7-(1-methyl-1H-pyrazol-4-yl)quinazolin-4-yl)benzo[d]thiazol-6-amine OR N-(5-(((3R,4R)-4-methoxytetrahydrofuran-3-yl)oxy)-7-(1-methyl-1H-pyrazol-4-yl)quinazolin-4-yl)benzo[d]thiazol-6-amine (37.4 mg). LC-MS: (ES, m/z): RT=1.258 min, LCMS: m/z=475 [M+1]. ¹H NMR (400 MHz, DMSO-d₆) δ 10.07 (s, 1H), 9.32 (s, 1H), 8.97 (d, J=2.1 Hz, 1H), 8.60 (s, 1H), 8.46 (s, 1H), 8.18-8.06 (m, 2H), 7.79 (dd, J=8.9, 2.2 Hz, 1H), 7.61 (dd, J=25.8, 1.6 Hz, 2H), 5.63 (d, J=3.3 Hz, 1H), 4.30-4.16 (m, 3H), 4.09 (dd, J=10.7, 3.3 Hz, 1H), 3.93 (s, 3H), 3.77 (dd, J=10.1, 2.1 Hz, 1H), 3.45 (s, 3H),

### Example A33: (3S,4S)-3-((4-(benzo[d]thiazol-6-ylamino)-7-(1-methyl-1H-pyrazol-4-yl)quinazolin-5-yl)oxy)-1-methylpiperidin-4-ol OR (3R,4R)-3-((4-(benzo[d]thiazol-6-ylamino)-7-(1-methyl-1H-pyrazol-4-yl)quinazolin-5-yl)oxy)-1-methylpiperidin-4-ol

### Example A34: (3R,4R)-3-((4-(benzo[d]thiazol-6-ylamino)-7-(1-methyl-1H-pyrazol-4-yl)quinazolin-5-yl)oxy)-1-methylpiperidin-4-ol OR (3S,4S)-3-((4-(benzo[d]thiazol-6-ylamino)-7-(1-methyl-1H-pyrazol-4-yl)quinazolin-5-yl)oxy)-1-methylpiperidin-4-ol

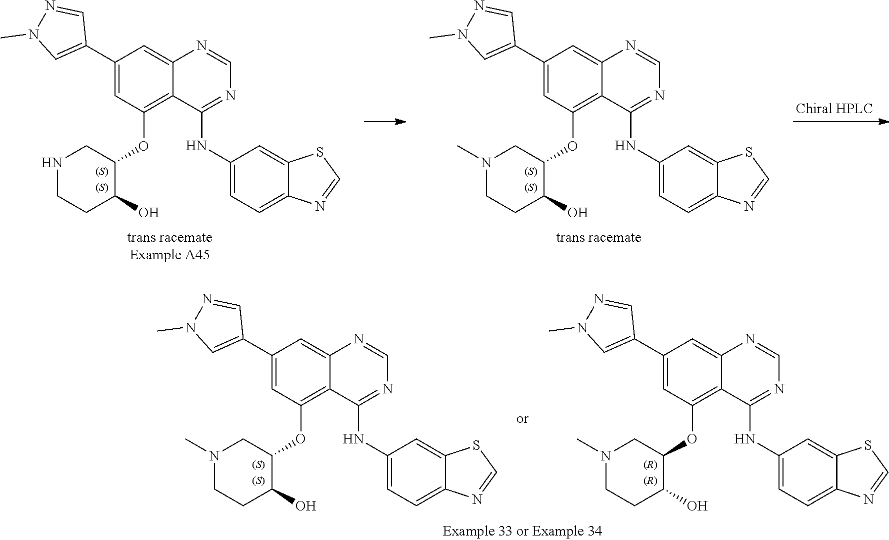

### Step 1: rac-(3S,4S)-3-((4-(benzo[d]thiazol-6-ylamino)-7-(1-methyl-1H-pyrazol-4-yl)quinazolin-5-yl)oxy)-1-methylpiperidin-4-ol

To a reaction vessel was added Example A45 (47.3 mg, 100 μmol, 1 eq), HCHO (30 mg, 1 mmol, 10 eq), MeOH (5 mL) and 1 drop of HOAc. The mixture was stirred at rt for 3 h. NaBH(OAc)₃ (42.3 mg, 200 μmol, 2 eq) was added. The reaction was stirred at rt for 18 h. The mixture was concentrated. The residue was purified by prep-TLC (DCM/MeOH=9:1) to afford the title compound (40 mg, 82.1%) as a white solid. LC-MS: (ES, m/z): RT=0.924 min, LCMS: m/z=488 [M+1].

### Step 2: Chiral Separation

rac-(3S,4S)-3-((4-(benzo[d]thiazol-6-ylamino)-7-(1-methyl-1H-pyrazol-4-yl)quinazolin-5-yl)oxy)-1-methylpiperidin-4-ol (50 mg, trans racemate) was purified by chiral HPLC: Column: CHIRALPAK IC, 2*25 cm, 5 μm; Mobile Phase A: Hex:DCM=3:1 (0.5% 2M NH₃-MeOH)-HPLC, Mobile Phase B: EtOH-HPLC; Flow rate: 20 mL/min; Gradient: 10% B to 10% B in 23.5 min; Wave Length: 220/254 nm; RT1 (min): 17.468; RT2 (min): 20.44; Sample Solvent: EtOH-HPLC afforded

Example A33: First eluting isomer, (3S,4S)-3-((4-(benzo[d]thiazol-6-ylamino)-7-(1-methyl-1H-pyrazol-4-yl)quinazolin-5-yl)oxy)-1-methylpiperidin-4-ol OR (3R,4R)-3-((4-(benzo[d]thiazol-6-ylamino)-7-(1-methyl-1H-pyrazol-4-yl)quinazolin-5-yl)oxy)-1-methylpiperidin-4-ol, (18.7 mg, 37.4%), as an off-white solid. LCMS: (ES, m/z): RT=0.974 min, LCMS: m/z=488 [M+1]; chiral-HPLC: R=4.898 min; ¹H NMR (400 MHz, DMSO-d6) δ 10.75 (s, 1H), 9.31 (s, 1H), 8.96 (d, J=2.1 Hz, 1H), 8.58 (s, 1H), 8.45 (s, 1H), 8.17-8.08 (m, 2H), 7.99 (dd, J=8.8, 2.1 Hz, 1H), 7.57 (d, J=1.4 Hz, 1H), 7.41 (d, J=1.5 Hz, 1H), 5.35 (d, J=4.0 Hz, 1H), 4.86 (s, 1H), 3.92 (s, 4H), 2.84 (s, 2H), 2.32 (s, 3H), 1.99 (dd, J=10.5, 6.1 Hz, 1H), 1.68-1.59 (m, 1H),

Example A34: Second eluting isomer, (3R,4R)-3-((4-(benzo[d]thiazol-6-ylamino)-7-(1-methyl-1H-pyrazol-4-yl)quinazolin-5-yl)oxy)-1-methylpiperidin-4-ol OR (3S,4S)-3-((4-(benzo[d]thiazol-6-ylamino)-7-(1-methyl-1H-pyrazol-4-yl)quinazolin-5-yl)oxy)-1-methylpiperidin-4-ol, (15.4 mg, 30.8%) as an off-white solid. LCMS: (ES, m/z): RT=0.974 min, LCMS: m/z=488 [M+1]; chiral-HPLC: R=6.120 min; ¹H NMR (400 MHz, DMSO-d6) δ 10.75 (s, 1H), 9.31 (s, 1H), 8.96 (d, J=2.1 Hz, 1H), 8.58 (s, 1H), 8.45 (s, 1H), 8.17-8.08 (m, 2H), 7.99 (dd, J=8.9, 2.2 Hz, 1H), 7.57 (d, J=1.4 Hz, 1H), 7.41 (d, J=1.7 Hz, 1H), 5.35 (d, J=3.9 Hz, 1H), 4.86 (d, J=4.2 Hz, 1H), 3.92 (s, 4H), 2.85 (s, 2H), 2.32 (s, 3H), 2.04-1.96 (m, 1H), 1.68-1.59 (m, 1H),

### Example A35: (3R,4R)-4-((4-(benzo[d]thiazol-6-ylamino)-7-(1-methyl-1H-pyrazol-4-yl)quinazolin-5-yl)oxy)-1-methylpiperidin-3-ol OR (3S,4S)-4-((4-(benzo[d]thiazol-6-ylamino)-7-(1-methyl-1H-pyrazol-4-yl)quinazolin-5-yl)oxy)-1-methylpiperidin-3-ol

### Example A36: (3S,4S)-4-((4-(benzo[d]thiazol-6-ylamino)-7-(1-methyl-1H-pyrazol-4-yl)quinazolin-5-yl)oxy)-1-methylpiperidin-3-ol OR (3R,4R)-4-((4-(benzo[d]thiazol-6-ylamino)-7-(1-methyl-1H-pyrazol-4-yl)quinazolin-5-yl)oxy)-1-methylpiperidin-3-ol

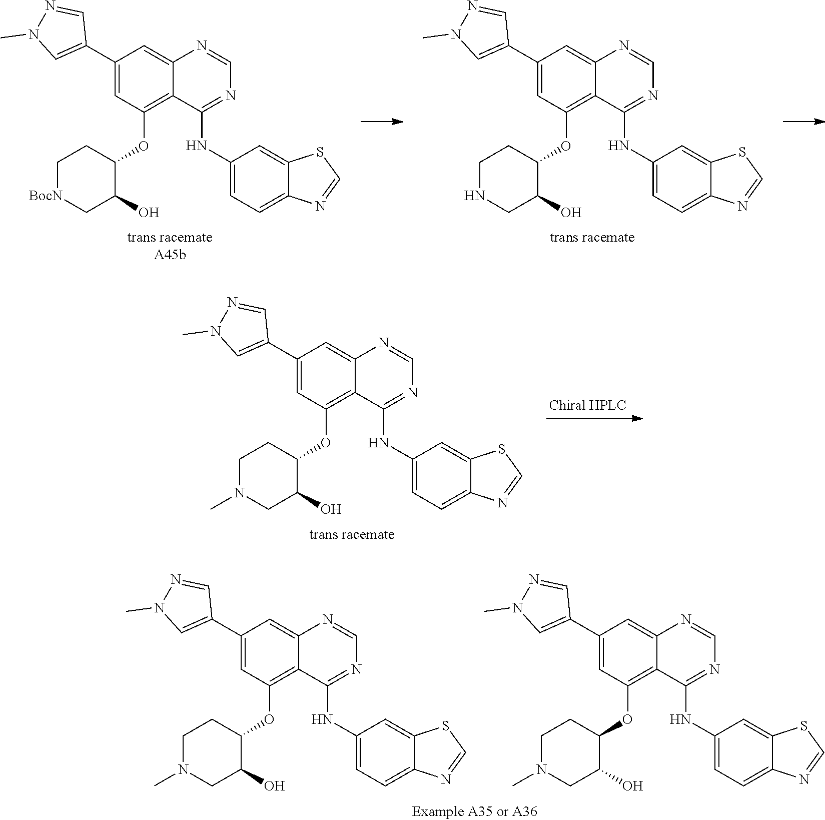 [SMILES: Cn1cc(-c2cc(O[C@H]3CCN(C(=O)OC(C)(C)C)C[C@@H]3O)c3c(Nc4ccc5ncsc5c4)ncnc3c2)cn1 ; Cn1cc(-c2cc(O[C@H]3CCNC[C@@H]3O)c3c(Nc4ccc5ncsc5c4)ncnc3c2)cn1 ; CN1CC[C@H](Oc2cc(-c3cnn(C)c3)cc3ncnc(Nc4ccc5ncsc5c4)c23)[C@@H](O)C1 ; CN1CC[C@H](Oc2cc(-c3cnn(C)c3)cc3ncnc(Nc4ccc5ncsc5c4)c23)[C@@H](O)C1 ; CN1CC[C@@H](Oc2cc(-c3cnn(C)c3)cc3ncnc(Nc4ccc5ncsc5c4)c23)[C@H](O)C1]

### Step 1: rac-(3S,4S)-4-((4-(benzo[d]thiazol-6-ylamino)-7-(1-methyl-1H-pyrazol-4-yl)quinazolin-5-yl)oxy)piperidin-3-ol

A solution of Example A45b (143 mg, 250 μmol, 1 eq), DCM (3 mL) and TFA (1 mL) was stirred at rt for 3 h. The mixture was concentrated, the residue was purified by prep-HPLC: Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 11% B to 41% B in 7 min; Wave Length: 254/220 nm to afford the title compound (60 mg, 50.8%) as a light yellow solid. LCMS (ESI, m/z): RT=1.044 min, LCMS: m/z=474 [M+1]; ¹H NMR (400 MHz, DMSO-d6) δ10.52 (s, 1H), 9.32 (s, 1H), 8.96 (d, J=2.1 Hz, 1H), 8.57 (s, 1H), 8.49 (s, 1H), 8.17 (d, J=0.9 Hz, 1H), 8.12 (d, J=8.8 Hz, 1H), 7.83 (dd, J=8.8, 2.2 Hz, 1H), 7.57 (d, J=1.4 Hz, 1H), 7.42 (d, J=1.7 Hz, 1H), 5.47 (d, J=5.3 Hz, 1H), 4.69 (s, 1H), 3.92 (s, 3H), 3.83 (tt, J=9.7, 5.1 Hz, 1H), 3.31 (s, OH), 3.22-3.13 (m, 1H), 2.95 (d, J=12.8 Hz, 1H), 2.72 (d, J=12.2 Hz, 1H), 2.49-2.42 (m, 1H), 2.37 (s, 1H), 1.57 (qd, J=11.9, 4.3 Hz, 1H),

### Step 2: rac-(3S,4S)-4-((4-(benzo[d]thiazol-6-ylamino)-7-(1-methyl-1H-pyrazol-4-yl)quinazolin-5-yl)oxy)-1-methylpiperidin-3-ol

To a mixture of rac-(3S,4S)-4-((4-(benzo[d]thiazol-6-ylamino)-7-(1-methyl-1H-pyrazol-4-yl)quinazolin-5-yl)oxy)piperidin-3-ol (47.3 mg, 100 μmol, 1 eq) and HCHO (30 mg, 1 mmol, 10 eq) in MeOH (5 mL) was added 1 drop of HOAc. The mixture was stirred at rt for 3 h then NaBH(OAc)₃ (42.3 mg, 200 μmol, 2 eq) was added to the mixture at rt. The reaction was stirred at rt for 18 h. The mixture was concentrated. The residue was purified by prep-TLC (DCM/MeOH=9:1) to afford the title compound (40 mg, 82.1%) as a white solid. LC-MS: (ES, m/z): RT=0.928 min, LCMS: m/z=488 [M+1].

### Step 3: Chiral Separation

rac-(3S,4S)-4-((4-(benzo[d]thiazol-6-ylamino)-7-(1-methyl-1H-pyrazol-4-yl)quinazolin-5-yl)oxy)-1-methylpiperidin-3-ol: (50 mg) was purified by chiral HPLC: Column: CHIRALPAK IF, 2*25 cm, 5 μm; Mobile Phase A: Hex:DCM=3:1 (0.5% 2M NH₃-MeOH)-HPLC, Mobile Phase B: DCM:EtOH=9:1-HPLC; Flow rate: 20 mL/min; Gradient: 30% B to 30% B in 18 min; Wave Length: 220/254 nm; RT1 (min): 6.552; RT2 (min): 14.56; to afford:

Example A35: (3R,4R)-4-((4-(benzo[d]thiazol-6-ylamino)-7-(1-methyl-1H-pyrazol-4-yl)quinazolin-5-yl)oxy)-1-methylpiperidin-3-ol OR (3S,4S)-4-((4-(benzo[d]thiazol-6-ylamino)-7-(1-methyl-1H-pyrazol-4-yl)quinazolin-5-yl)oxy)-1-methylpiperidin-3-ol. (20 mg, 40%) as a white solid. LCMS: (ES, m/z): RT=0.867 min, LCMS: m/z=488 [M+1]; chiral-HPLC: R=1.226 min; ¹H NMR (400 MHz, DMSO-d6) δ10.46 (s, 1H), 9.31 (s, 1H), 8.94 (d, J=2.2 Hz, 1H), 8.56 (s, 1H), 8.48 (s, 1H), 8.17 (s, 1H), 8.11 (d, J=8.8 Hz, 1H), 7.82 (dd, J=8.9, 2.2 Hz, 1H), 7.57 (d, J=1.5 Hz, 1H), 7.41 (d, J=1.6 Hz, 1H), 5.55 (d, J=5.5 Hz, 1H), 4.95-4.40 (m, 1H), 3.99 (tt, J=9.9, 5.1 Hz, 1H), 3.91 (s, 3H), 3.03 (ddd, J=10.8, 4.7, 1.9 Hz, 1H), 2.78 (d, J=11.6 Hz, 1H), 2.39-2.32 (m, 1H), 2.25 (s, 4H), 2.03 (t, J=10.5 Hz, 1H), 1.74 (qd, J=12.3, 4.2 Hz, 1H),

Example A36: (3S,4S)-4-((4-(benzo[d]thiazol-6-ylamino)-7-(1-methyl-1H-pyrazol-4-yl)quinazolin-5-yl)oxy)-1-methylpiperidin-3-ol OR (3R,4R)-4-((4-(benzo[d]thiazol-6-ylamino)-7-(1-methyl-1H-pyrazol-4-yl)quinazolin-5-yl)oxy)-1-methylpiperidin-3-ol (20 mg, 40%) as a white solid. LCMS: (ES, m/z): RT=0.862 min, LCMS: m/z=488 [M+1]; chiral-HPLC: R=2.271 min; ¹H NMR (400 MHz, DMSO-d6) δ 10.46 (s, 1H), 9.31 (s, 1H), 8.94 (d, J=2.1 Hz, 1H), 8.57 (s, 1H), 8.48 (s, 1H), 8.17 (d, J=0.8 Hz, 1H), 8.11 (d, J=8.8 Hz, 1H), 7.82 (dd, J=8.9, 2.2 Hz, 1H), 7.57 (d, J=1.4 Hz, 1H), 7.41 (d, J=1.6 Hz, 1H), 5.55 (d, J=5.5 Hz, 1H), 4.60 (td, J=9.9, 4.7 Hz, 1H), 3.99 (tt, J=9.9, 5.1 Hz, 1H), 3.92 (s, 3H), 3.08-2.99 (m, 1H), 2.78 (d, J=11.5 Hz, 1H), 2.35 (d, J=12.1 Hz, 1H), 2.25 (s, 4H), 2.03 (t, J=10.5 Hz, 1H), 1.74 (qd, J=11.9, 4.1 Hz, 1H),

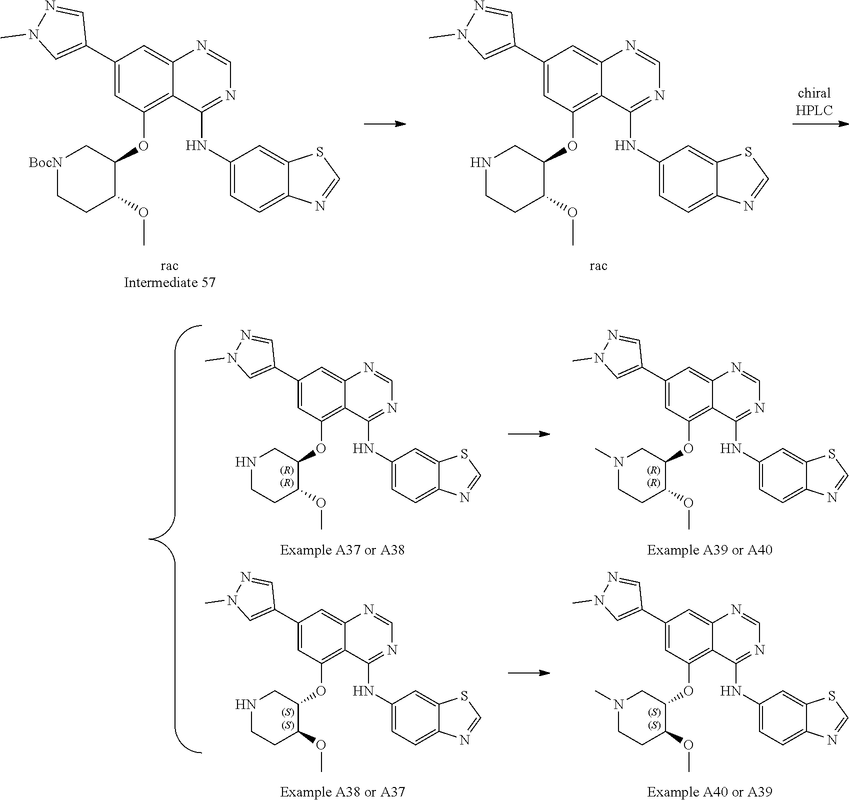

Example A37: N-(5-(((3S,4S)-4-methoxypiperidin-3-yl)oxy)-7-(1-methyl-1H-pyrazol-4-yl)quinazolin-4-yl)benzo[d]thiazol-6-amine ORN-(5-(((3R,4R)-4-methoxypiperidin-3-yl)oxy)-7-(1-methyl-1H-pyrazol-4-yl)quinazolin-4-yl)benzo[d]thiazol-6-amine.

Example A38: N-(5-(((3S,4S)-4-methoxypiperidin-3-yl)oxy)-7-(1-methyl-1H-pyrazol-4-yl)quinazolin-4-yl)benzo[d]thiazol-6-amine ORN-(5-(((3R,4R)-4-methoxypiperidin-3-yl)oxy)-7-(1-methyl-1H-pyrazol-4-yl)quinazolin-4-yl)benzo[d]thiazol-6-amine.

Into a 20-mL pressure tank reactor purged and maintained with an inert atmosphere of nitrogen, was placed Intermediate BP-57 (200.00 mg, 0.340 mmol, 1.00 eq), DCM (2.00 mL, 31.460 mmol, 92.45 eq), TFA (2.00 mL, 26.926 mmol, 79.12 eq). The resulting solution was stirred for 3 hr. at room temperature. TCL show SM disappear. The reaction was then concentrated under vacuum to afford 200 mg of a light yellow solid as a mixture of Examples A37 and A38. LC-MS: (ES, m/z): LCMS: m/z=488 [M+1]

Chiral Separation: The diastereomers were separated using chiral HPLC Column: CHIRALPAK IH-3, 4.6*50 mm, 3.0 um; Mobile Phase A: MtBE (0.1% DEA):EtOH=80:20; Flow rate: 1 mL/min; Gradient: 0% B to 0% B; Injection Volume: Sul mL to afford:

Example A37: First Eluting Peak, N-(5-(((3R,4R)-4-methoxypiperidin-3-yl)oxy)-7-(1-methyl-1H-pyrazol-4-yl)quinazolin-4-yl)benzo[d]thiazol-6-amine, OR N-(5-(((3S,4S)-4-methoxypiperidin-3-yl)oxy)-7-(1-methyl-1H-pyrazol-4-yl)quinazolin-4-yl)benzo[d]thiazol-6-amine (30.2 mg) as a white solid. (ES, m/z): RT=0.940 min, LCMS: m/z=488 [M+1]¹H NMR (400 MHz, DMSO-d6) δ 10.57 (s, 1H), 9.32 (s, 1H), 8.91 (d, J=2.1 Hz, 1H), 8.55 (s, 1H), 8.46 (s, 1H), 8.15 (d, J=0.8 Hz, 1H), 8.10 (d, J=8.8 Hz, 1H), 7.94 (dd, J=8.9, 2.2 Hz, 1H), 7.58 (d, J=1.5 Hz, 1H), 7.47 (d, J=1.7 Hz, 1H), 4.88 (s, 1H), 3.92 (s, 3H), 3.78 (s, 1H), 3.36 (s, 3H), 3.30 (s, 2H), 2.96 (s, 2H), 2.76 (s, 1H), 2.07 (s, 1H), 1.60 (s, 1H),

Example A38: Second Eluting Peak: N-(5-(((3S,4S)-4-methoxypiperidin-3-yl)oxy)-7-(1-methyl-1H-pyrazol-4-yl)quinazolin-4-yl)benzo[d]thiazol-6-amine OR N-(5-(((3R,4R)-4-methoxypiperidin-3-yl)oxy)-7-(1-methyl-1H-pyrazol-4-yl)quinazolin-4-yl)benzo[d]thiazol-6-amine (21.4 mg) as a white solid. LC-MS (ES, m/z): RT=0.957 min, LCMS: m/z=488 [M+1], ¹H NMR (400 MHz, DMSO-d6) δ 10.63 (s, 1H), 9.32 (s, 1H), 8.91 (d, J=2.1 Hz, 1H), 8.55 (s, 1H), 8.46 (s, 1H), 8.15 (d, J=0.8 Hz, 1H), 8.09 (d, J=8.8 Hz, 1H), 7.95 (dd, J=8.8, 2.2 Hz, 1H), 7.58 (d, J=1.5 Hz, 1H), 7.47 (d, J=1.7 Hz, 1H), 4.91-4.82 (m, 1H), 3.92 (s, 3H), 3.75 (td, J=6.7, 4.0 Hz, 1H), 3.36 (s, 3H), 2.92 (td, J=14.1, 12.6, 7.3 Hz, 2H), 2.73-2.63 (m, 1H), 2.05 (s, 1H), 2.10-2.00 (m, 1H), 1.57 (s, 1H)

### Example A39: N-(5-(((3R,4R)-4-methoxy-1-methylpiperidin-3-yl)oxy)-7-(1-methyl-1H-pyrazol-4-yl)quinazolin-4-yl)benzo[d]thiazol-6-amine OR N-(5-(((3S,4S)-4-methoxy-1-methylpiperidin-3-yl)oxy)-7-(1-methyl-1H-pyrazol-4-yl)quinazolin-4-yl)benzo[d]thiazol-6-amine

Into a 20-mL pressure tank reactor purged and maintained with an inert atmosphere of nitrogen, was placed Example A37 (18.04 mg, 0.287 mmol, 2 eq). The resulting solution was stirred for 1 hr. at 0° C. The reaction was then quenched by the addition of 10 mL of water/ice. The solids were filtered out. The resulting solution was extracted with 3×10 mL of EA and concentrated under vacuum. The residue was purified by silica gel column, eluting with DCM/MeOH (20/1). The crude product was purified by Flash-Prep-HPLC Column: XBridge Prep OBD C18 Column, 30*150 mm, 5 μm; Mobile Phase A: Water (10 MMOL/L NH₄HCO₃+0.1% NH₃·H₂O), Mobile Phase B: ACN; Flow rate: 60 mL/min; Gradient: 36% B to 56% B in 7 min, 56% B; Wave Length: 254; 220 nm; RT1 (min): 5.93; to afford the title compound as a white solid. LC-MS (ES, m/z): RT=1.107 min. LCMS: m/z=502 [M+1], ¹H NMR (300 MHz, DMSO-d6) δ 10.73 (s, 1H), 9.32 (s, 1H), 8.93 (dd, J=5.1, 2.1 Hz, 1H), 8.57 (d, J=1.6 Hz, 1H), 8.47 (s, 1H), 8.17-8.08 (m, 2H), 7.96 (dt, J=8.9, 2.8 Hz, 1H), 7.59 (d, J=1.4 Hz, 1H), 7.50 (d, J=1.6 Hz, 1H), 5.16 (s, 1H), 3.92 (s, 3H), 3.67-3.60 (m, 1H), 2.95 (d, J=12.4 Hz, 1H), 2.69 (s, 1H), 2.30 (s, 3H), 1.96 (t. J=12.6 Hz, 1H), 1.77 (d, J=14.0 Hz, 1H).

### Example A40: N-(5-(((3S,4S)-4-methoxy-1-methylpiperidin-3-yl)oxy)-7-(1-methyl-1H-pyrazol-4-yl)quinazolin-4-yl)benzo[d]thiazol-6-amine OR N-(5-(((3R,4R)-4-methoxy-1-methylpiperidin-3-yl)oxy)-7-(1-methyl-1H-pyrazol-4-yl)quinazolin-4-yl)benzo[d]thiazol-6-amine

Into a 20-mL pressure tank reactor purged and maintained with an inert atmosphere of nitrogen, was placed Example A38 (70.00 mg, 0.144 mmol, 1.00 eq), HCHO (43.11 mg, 1.436 mmol, 10 eq), CH3OH (5.00 mL). The resulting solution was stirred for 1 hr. at 0° C. The reaction was then quenched by the addition of 10 mL of water/ice The solids were filtered out The resulting solution was extracted with 3×10 mL of EA concentrated under vacuum. The residue was purified by silica gel column, eluting with DCM/MeOH (20/1). The product was further purified by Flash-Prep-HPLC Column: XBridge Prep OBD C18 Column, 30*150 mm, 5 μm; Mobile Phase A: Water (10 MMOL/L NH₄HCO₃+0.1% NH₃·H₂O), Mobile Phase B: ACN; Flow rate: 60 mL/min; Gradient: 36% B to 56% B in 7 min, Wave Length: 254; 220 nm to afford the title compound (31.5 mg, 43.73%) as a white solid. LC-MS: (ES, m/z): RT=1.090 min, LCMS: m/z=502 [M+1], ¹H NMR (300 MHz, DMSO-d6) δ 10.72 (s, 1H), 9.32 (s, 1H), 8.93 (dd, J=5.0, 2.0 Hz, 1H), 8.57 (d, J=1.7 Hz, 1H), 8.46 (s, 1H), 8.17-8.08 (m, 2H), 7.96 (ddd, J=8.9, 3.5, 2.1 Hz, 1H), 7.59 (d, J=1.5 Hz, 1H), 7.50 (d, J=1.6 Hz, 1H), 5.16 (s, 1H), 3.92 (s, 3H), 3.64 (d, J=3.9 Hz, 1H), 2.95 (d, J=11.5 Hz, 1H), 2.71 (d, J=13.1 Hz, 1H), 2.58 (s, 1H), 2.37 (d, J=10.8 Hz, 1H), 2.30 (s, 3H), 1.96 (dd, J=14.5, 10.5 Hz, 1H), 1.77 (d, J=13.9 Hz, 1H),

### Examples A41-A44

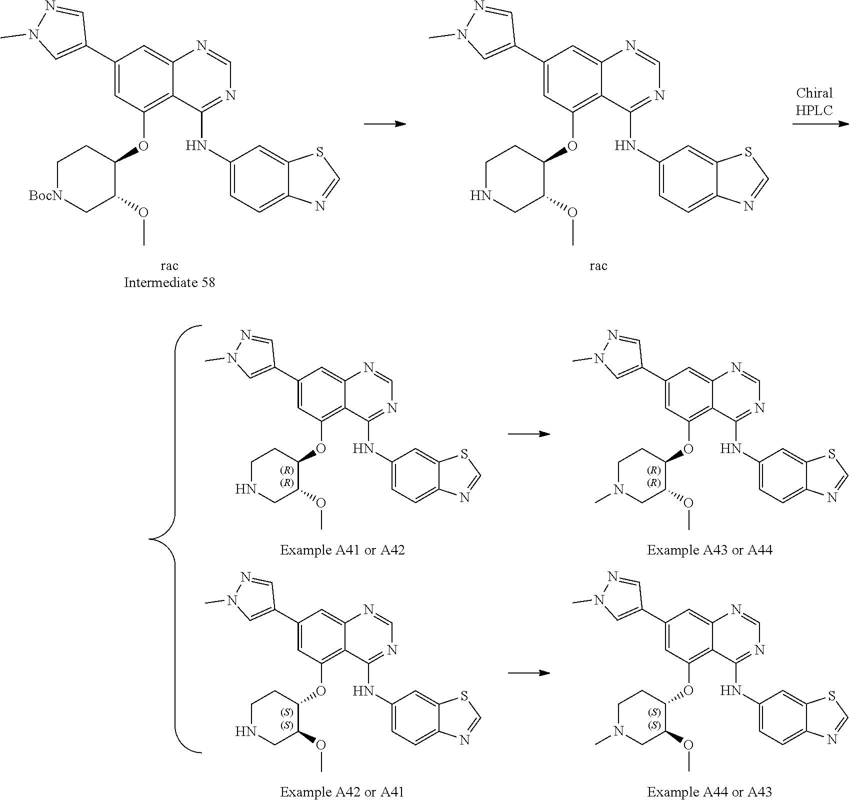

### Example A41: N-(5-(((3R,4R)-3-methoxypiperidin-4-yl)oxy)-7-(1-methyl-1H-pyrazol-4-yl)quinazolin-4-yl)benzo[d]thiazol-6-amine OR N-(5-(((3S,4S)-3-methoxypiperidin-4-yl)oxy)-7-(1-methyl-1H-pyrazol-4-yl)quinazolin-4-yl)benzo[d]thiazol-6-amine

### Example A42: N-(5-(((3S,4S)-3-methoxypiperidin-4-yl)oxy)-7-(1-methyl-1H-pyrazol-4-yl)quinazolin-4-yl)benzo[d]thiazol-6-amine OR N-(5-(((3R,4R)-3-methoxypiperidin-4-yl)oxy)-7-(1-methyl-1H-pyrazol-4-yl)quinazolin-4-yl)benzo[d]thiazol-6-amine

Step 1: Into a 20-mL pressure tank reactor purged and maintained with an inert atmosphere of nitrogen, was placed Intermediate BP-58 (200.00 mg, 0.340 mmol, 1.00 eq), DCM (2.00 mL, 31.460 mmol, 92.45 eq), TFA (2.00 mL, 26.926 mmol, 79.12 eq). The resulting solution was stirred for 3 hr. at room temperature. The reaction was then concentrated under vacuum to afford 200 mg of a light yellow solid as a mixture of Examples A41 and A42. LC-MS: (ES, m/z): LCMS: m/z=488 [M+1]

### Step 2: Chiral Separation

The diastereomers were separated using chiral HPLC Column: CHIRALPAK IF-3, 4.6*50 mm. 3.0 um; Mobile Phase A. MtBE (0.1% DEA): MeOH=90:10. Flow rate. 1 mL/min: Gradient: 0% B to 0% B: Injection Volume: 5 ul mL to afford:

Example A41: First eluting isomer, N-(5-(((3R,4R)-3-methoxypiperidin-4-yl)oxy)-7-(1-methyl-1H-pyrazol-4-yl)quinazolin-4-yl)benzo[d]thiazol-6-amine OR N-(5-(((3S,4S)-3-methoxypiperidin-4-yl)oxy)-7-(1-methyl-1H-pyrazol-4-yl)quinazolin-4-yl)benzo[d]thiazol-6-amine as a white solid. LC-MS: (ES, m/z): RT=0.799 min, LCMS: m/z=488 [M+1], ¹H NMR (400 MHz, DMSO-d6) δ 10.25 (s, 1H), 9.34 (s, 1H), 8.91 (d, J=2.2 Hz, 1H), 8.57 (d, J=1.8 Hz, 1H), 8.46 (s, 1H), 8.18-8.10 (m, 21H), 7.79 (dd, J=8.8, 2.2 Hz, 1H), 7.60 (d, J=1.5 Hz, 1H), 7.47 (d, J=1.6 Hz, 1H), 4.95 (s, 1H), 3.92 (s, 3H), 3.76 (s, 1H), 3.06 (s, 2H), 2.83 (s, 1H), 2.37 (s, 1H), 1.85 (s, 1H), 1.24 (s, 1H),

Example A42: Second eluting isomer, N-(5-(((3S,4S)-3-methoxypiperidin-4-yl)oxy)-7-(1-methyl-1H-pyrazol-4-yl)quinazolin-4-yl)benzo[d]thiazol-6-amine OR N-(5-(((3R,4R)-3-methoxypiperidin-4-yl)oxy)-7-(1-methyl-1H-pyrazol-4-yl)quinazolin-4-yl)benzo[d]thiazol-6-amine (2.5 mg) as a white solid. LCMS (ES, m/z): RT=0.799 min, LCMS: m/z=488 [M+1]¹H NMR (400 MHz, DMSO-d6) δ 10.33 (s, 1H), 9.33 (s, 1H), 8.93 (dd, J=4.8, 2.1 Hz, 1H), 8.57 (s, 1H), 8.47 (s, 1H), 8.18-8.10 (m, 2H), 7.79 (dd. J=8.8, 2.2 Hz, 1H), 7.59 (d, J=1.4 Hz, 1H), 7.46 (d, J=1.6 Hz, 1H), 4.90 (td. J=9.3, 4.7 Hz, 1H), 3.92 (s, 31H), 3.70 (td, J=8.7, 4.5 Hz, 1H), 3.44-3.33 (m, 1H), 3.34 (s, 3H), 3.01 (d, J=12.8 Hz, 1H), 2.75 (t, J=11.2 Hz, 1H), 2.35 (s, OH), 1.79 (d, J=11.3 Hz, 1H),

### Example A43: N-(5-(((3R,4R)-3-methoxy-1-methylpiperidin-4-yl)oxy)-7-(1-methyl-1H-pyrazol-4-yl)quinazolin-4-yl)benzo[d]thiazol-6-amine OR N-(5-(((3S,4S)-3-methoxy-1-methylpiperidin-4-yl)oxy)-7-(1-methyl-1H-pyrazol-4-yl)quinazolin-4-yl)benzo[d]thiazol-6-amine

Into a 8-mL pressure tank reactor purged and maintained with an inert atmosphere of nitrogen, was placed Example A41 (10.00 mg, 0.021 mmol, 1.00 eq), methanol (2.00 mL), HCHO (1.85 mg, 0.063 mmol, 3.00 eq). STAB (13.04 mg, 0.063 mmol, 3.00 eq) The resulting solution was stirred for 1 hr at 0° C. The reaction was then quenched by the addition of 10 mL of water/ice. The solids were filtered out. The resulting solution was extracted with 3×10 mL of EA concentrated under vacuum. The residue was purified by silica gel column, eluting with DCM/MeOH (20/1). The crude product was purified by Flash-Prep-HPLC to afford the title compound (6.1 ng, 59.29%) as a white solid. LC-MS: (ES, m/z): RT=0.990 min, LCMS: m/z=502[M+1], ¹H NMR (400 MHz, DMSO-d6) δ 10.33 (s, 2H), 9.33 (s, 2H), 8.92 (d, J=2.2 Hz, 2H), 8.57 (s, 2H), 8.47 (s, 2H), 8.19-8.10 (m, 4H), 7.83 (dd, J=8.8, 2.2 Hz, 2H), 7.58 (d, J=1.4 Hz, 2H), 7.45 (d, J=1.6 Hz, 2H), 4.78 (s, 1H), 4.78 (dd, J=19.1, 4.8 Hz, 1H), 3.92 (s, 6H), 3.90-3.77 (m, 2H), 3.31 (d, J=7.1 Hz, 6H), 3.20 (s, 1H), 2.79 (d, J=11.2 Hz, 2H), 2.28 (s, 61H), 2.23 (d, J=12.1 Hz, 1H), 1.99 (t, J=10.4 Hz, 2H), 1.95-1.83 (m, 1H),

### Example A44: N-(5-(((3S,4S)-3-methoxy-1-methylpiperidin-4-yl)oxy)-7-(1-methyl-1H-pyrazol-4-yl)quinazolin-4-yl)benzo[d]thiazol-6-amine OR N-(5-(((3R,4R)-3-methoxy-1-methylpiperidin-4-yl)oxy)-7-(1-methyl-1H-pyrazol-4-yl)quinazolin-4-yl)benzo[d]thiazol-6-amine

Into a 8-mL pressure tank reactor purged and maintained with an inert atmosphere of nitrogen, was placed Example A42 (10.00 mg, 0.021 mmol, 1.00 eq), methanol (2.00 mL), HCHO (1.85 mg, 0.062 mmol, 3 eq), STAB (13.04 mg, 0.062 mmol, 3 eq). The resulting solution was stirred for 1 hr. at 0° C. The reaction was then quenched by the addition of 10 mL of water/ice. The solids were filtered out. The resulting solution was extracted with 3×10 mL of EA concentrated under vacuum. The residue was purified by silica gel column, eluting with DCM/MeOH (20/1). The product was further purified by Flash-Prep-HPLC to afford the title compound (6.9 Ing, 67.07%) as a white solid. LC-MS: (ES, m/z): RT=0.973 min, LCMS: m/z=502 [M+1], ¹H NMR (400 MHz, DMSO-d6) δ 10.33 (s, 1H), 9.33 (s, 1H), 8.92 (d, J=2.2 Hz, 1H), 8.57 (s, 1H), 8.47 (s, 1H), 8.19-8.10 (m, 2H), 7.83 (dd, J=8.9, 2.2 Hz, 1H), 7.58 (d, J=1.5 Hz, 1H), 7.45 (d, J=1.6 Hz, 1H), 4.78 (s, 1H), 3.92 (s, 3H), 3.82 (td, J=9.1, 4.6 Hz, 1H), 3.32 (s, 4H), 2.79 (d, J=11.4 Hz, 1H), 2.32 (s, 1H), 2.28 (s, 3H), 2.23 (d, J=11.8 Hz, 1H), 1.99 (t, J=10.3 Hz, 1H), 1.89 (qd, J=11.2, 4.1 Hz, 1H),

### Example A45: (3S,4S)-3-((4-(benzo[d]thiazol-6-ylamino)-7-(1-methyl-1H-pyrazol-4-yl)quinazolin-5-yl)oxy)piperidin-4-ol

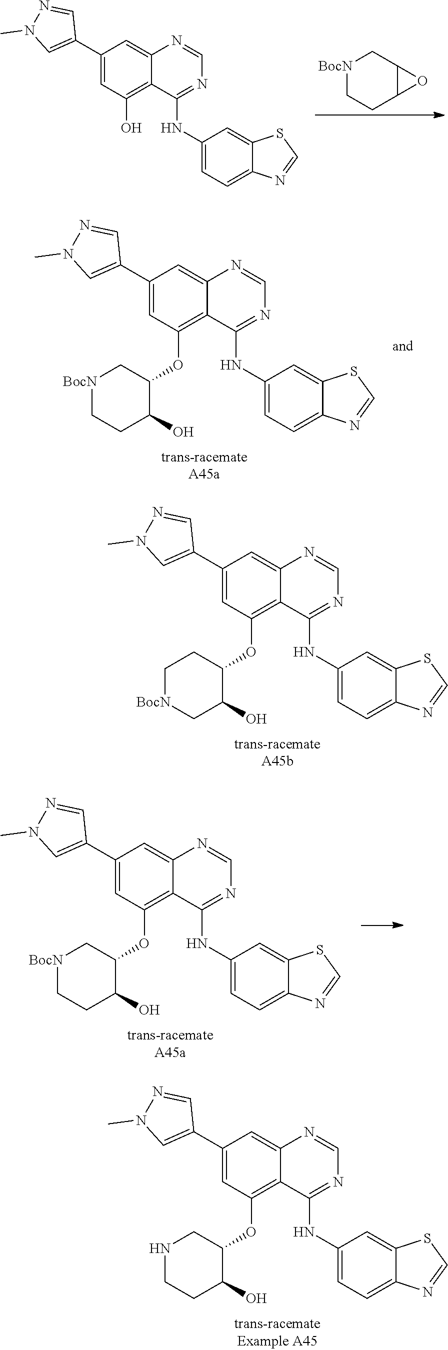 [SMILES: Cn1cc(-c2cc(O)c3c(Nc4ccc5ncsc5c4)ncnc3c2)cn1 ; CC(C)(C)OC(=O)N1CCC2OC2C1 ; Cn1cc(-c2cc(O[C@H]3CN(C(=O)OC(C)(C)C)CC[C@@H]3O)c3c(Nc4ccc5ncsc5c4)ncnc3c2)cn1 ; Cn1cc(-c2cc(O[C@H]3CCN(C(=O)OC(C)(C)C)C[C@@H]3O)c3c(Nc4ccc5ncsc5c4)ncnc3c2)cn1 ; Cn1cc(-c2cc(O[C@H]3CN(C(=O)OC(C)(C)C)CC[C@@H]3O)c3c(Nc4ccc5ncsc5c4)ncnc3c2)cn1 ; Cn1cc(-c2cc(O[C@H]3CNCC[C@@H]3O)c3c(Nc4ccc5ncsc5c4)ncnc3c2)cn1]

### Step 1: Mixture of tert-butyl (3S,4S)-3-((4-(benzo[d]thiazol-6-ylamino)-7-(1-methyl-1H-pyrazol-4-yl)quinazolin-5-yl)oxy)-4-hydroxypiperidine-1-carboxylate and tert-butyl (3S,4S)-4-((4-(benzo[d]thiazol-6-ylamino)-7-(1-methyl-1H-pyrazol-4-yl)quinazolin-5-yl)oxy)-3-hydroxypiperidine-1-carboxylate

A mixture of intermediate BP-62 (748 mg, 2 mmol, 1 eq) and tert-butyl 7-oxa-3-azabicyclo[4.1.0]heptane-3-carboxylate (1.99 g, 10 mmol, 5 eq) in dioxane (12 mL) was added NaOH (3 mL, 6 mmol, 3 eq, 2M). The mixture was heated to 100° C. for 18 h. After cooling to rt, the mixture was concentrated to afford a mixture of A45a and A45b as a yellow syrup. The isomers were separated by prep-HPLC: Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 35% B to 55% B in 7 min; Wave Length: 254/220 nm to afford: A45a: tert-butyl (3S,4S)-3-((4-(benzo[d]thiazol-6-ylamino)-7-(1-methyl-1H-pyrazol-4-yl)quinazolin-5-yl)oxy)-4-hydroxypiperidine-1-carboxylate (120 mg, 10.5%) as a light yellow solid. LC-MS: (ES, m/z): RT=1.279 min, LCMS: m/z=574 [M+1]

A45b: tert-butyl (3S,4S)-4-((4-(benzo[d]thiazol-6-ylamino)-7-(1-methyl-1H-pyrazol-4-yl)quinazolin-5-yl)oxy)-3-hydroxypiperidine-1-carboxylate (300 mg, 26.3%) as a light yellow solid. LC-MS: (ES, m/z): RT=1.320 min, LCMS: m/z=574 [M+1]

### Step 2: (3S,4S)-3-((4-(benzo[d]thiazol-6-ylamino)-7-(1-methyl-1H-pyrazol-4-yl)quinazolin-5-yl)oxy)piperidin-4-ol

A solution of A45a (114 mg, 200 μmol, 1 eq) in DCM (3 mL) and TFA (1 mL) was stirred at rt for 3 h. The mixture was concentrated, and the residue was purified by prep-HPLC: Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 12% B to 42% B in 7 min; Wave Length: 254/220 nm to afford the resulted in the title compound (50 mg, 53.2%) as a light yellow solid. LC-MS: (ES, m/z): RT=3.895 min, LCMS: m/z=474 [M+1]; ¹H NMR (400 MHz, DMSO-d6) δ 10.65 (s, 1H), 9.31 (s, 1H), 8.96 (d, J=2.1 Hz, 1H), 8.56 (s, 1H), 8.45 (s, 1H), 8.15 (d, J=0.8 Hz, 1H), 8.09 (d, J=8.8 Hz, 1H), 7.96 (dd, J=8.9, 2.2 Hz, 1H), 7.56 (d, J=1.4 Hz, 1H), 7.41 (d, J=1.6 Hz, 1H), 5.37 (d, J=5.1 Hz, 1H), 4.59 (d, J=3.5 Hz, 1H), 3.95-3.92 (m, 1H), 3.92 (s, 3H), 3.42 (dd, J=12.4, 3.5 Hz, 1H), 3.31 (s, 1H), 3.00-2.92 (m, 1H), 2.71 (dd, J=12.5, 7.9 Hz, 1H), 2.65-2.44 (m, 1H), 2.00 (d, J=12.6 Hz, 1H), 1.59-1.48 (m, 1H),

### Example A47: N-(4-(benzo[d]thiazol-6-ylamino)-7-(1-methyl-1H-pyrazol-3-yl)quinazolin-6-yl)-3-(piperidin-1-yl)propanamide

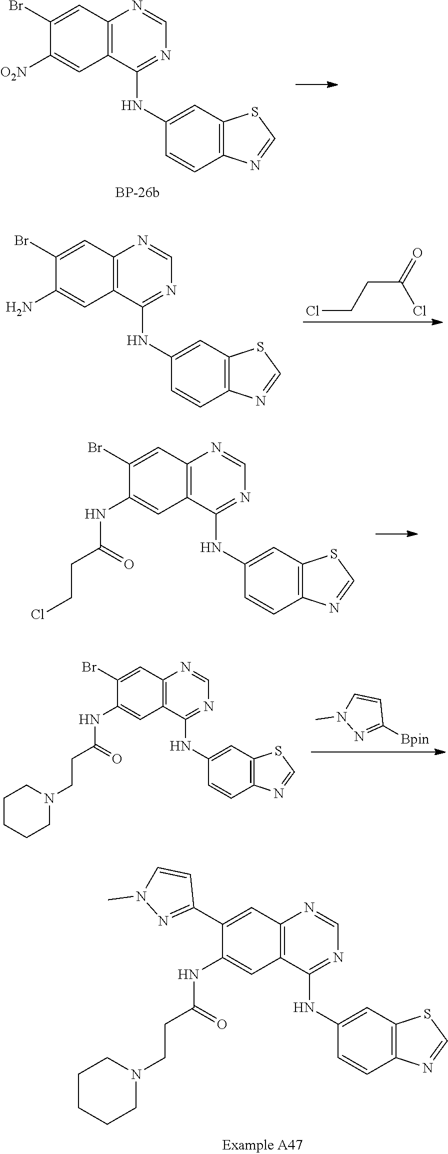

### Step 1: N4-(benzo[d]thiazol-6-yl)-7-bromoquinazoline-4,6-diamine

Ammonium chloride (212 mg, 3.97 mmol) and iron (221 mg, 3.97 mmol) was added to Intermediate BP-26b (200 mg, 497 umol) in EtOH/H₂O (4 ml/1 ml) at rt. The resulting mixture was heated to 80° C. for 1 hour. The reaction was filtered and the filtrate was concentrated under vacuum. The residue was purified by TLC (DCM:MeOH=15:1) to afford the title compound (120 mg) as a light yellow solid.

### Step 2: N-(4-(benzo[d]thiazol-6-ylamino)-7-bromoquinazolin-6-yl)-3-chloropropanamide

A solution of N4-(benzo[d]thiazol-6-yl)-7-bromoquinazoline-4,6-diamine (100 mg, 268 umol) in 3-chloropropanoyl chloride (2 ml) was heated to 50° C. for 1 hour. The reaction was extracted with EA (10 mL×3). The organic layers were combined, dried over anhydrous sodium sulfate and concentrated under vacuum to afford the title compound (100 mg) as a white solid. LC-MS: (ES, m/z): RT=2.008 min, LCMS: m/z=462 [M−1]

### Step 3: N-(4-(benzo[d]thiazol-6-ylamino)-7-bromoquinazolin-6-yl)-3-(piperidin-1-yl)propanamide

A solution of N-(4-(benzo[d]thiazol-6-ylamino)-7-bromoquinazolin-6-yl)-3-chloropropanamide (100 mg, 216 umol) in piperidine (2 ml) was heated to 60° C. for 1 hour. The reaction was concentrated under vacuum and purified by prep-TLC (MeOH:DCM=1:10) to afford the title compound (90 mg) as a light yellow solid. LC-MS: (ES, m/z): RT=2.394 min, LCMS: m/z=511 [M−1]

### Step 4: N-(4-(benzo[d]thiazol-6-ylamino)-7-(1-methyl-1H-pyrazol-3-yl)quinazolin-6-yl)-3-(piperidin-1-yl)propanamide

K₂CO₃ (49 mg, 350 umol) and Pd(dppf)Cl₂ (14.2 mg, 17.5 umol) were added to N-(4-(benzo[d]thiazol-6-ylamino)-7-bromoquinazolin-6-yl)-3-(piperidin-1-yl)propanamide (90 mg, 175 umol) and 1-methyl-3-(4,4,5,5-tetramethyl-1,3,2-dioxaborolan-2-yl)-1H-pyrazole (36.4 mg, 175 umol) in dioxane/water (3 mL/0.8 mL) at rt. The reaction was heated at 100° C. The reaction was concentrated under vacuum and purified by Pre-HPLC: Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 32% B to 47% B in 7 min; Wave Length: 254/220 nm; to afford the title compound 18.5 mg as an off-white solid. LC-MS: (ES, m/z): RT=1.556 min, LCMS: m/z=513 [M+1]; H-NMR-PH-BPM-B2343-0: 1H NMR (400 MHz, DMSO-d6) δ 11.31 (s, 1H), 10.11 (s, 1H), 9.31 (s, 1H), 9.21 (s, 1H), 8.69 (d, J=2.1 Hz, 1H), 8.57 (s, 1H), 8.19 (s, 1H), 8.09 (d, J=8.8 Hz, 1H), 7.98-7.88 (m, 2H), 7.09 (d, J=2.4 Hz, 1H), 4.02 (s, 3H), 2.69 (s, 2H), 2.64 (d, J=6.4 Hz, 2H), 2.40 (s, 4H), 1.44 (s, 4H), 1.36 (s, 2H),

### Example 48: (3S,4S)-4-((4-(benzo[d]thiazol-6-ylamino)-7-(3-methoxy-1-methyl-1H-pyrazol-4-yl)quinazolin-5-yl)oxy)tetrahydrofuran-3-ol OR (3R,4R)-4-((4-(benzo[d]thiazol-6-ylamino)-7-(3-methoxy-1-methyl-1H-pyrazol-4-yl)quinazolin-5-yl)oxy)tetrahydrofuran-3-ol

### Example 49: (3R,4R)-4-((4-(benzo[d]thiazol-6-ylamino)-7-(3-methoxy-1-methyl-1H-pyrazol-4-yl)quinazolin-5-yl)oxy)tetrahydrofuran-3-ol OR (3S,4S)-4-((4-(benzo[d]thiazol-6-ylamino)-7-(3-methoxy-1-methyl-1H-pyrazol-4-yl)quinazolin-5-yl)oxy)tetrahydrofuran-3-ol

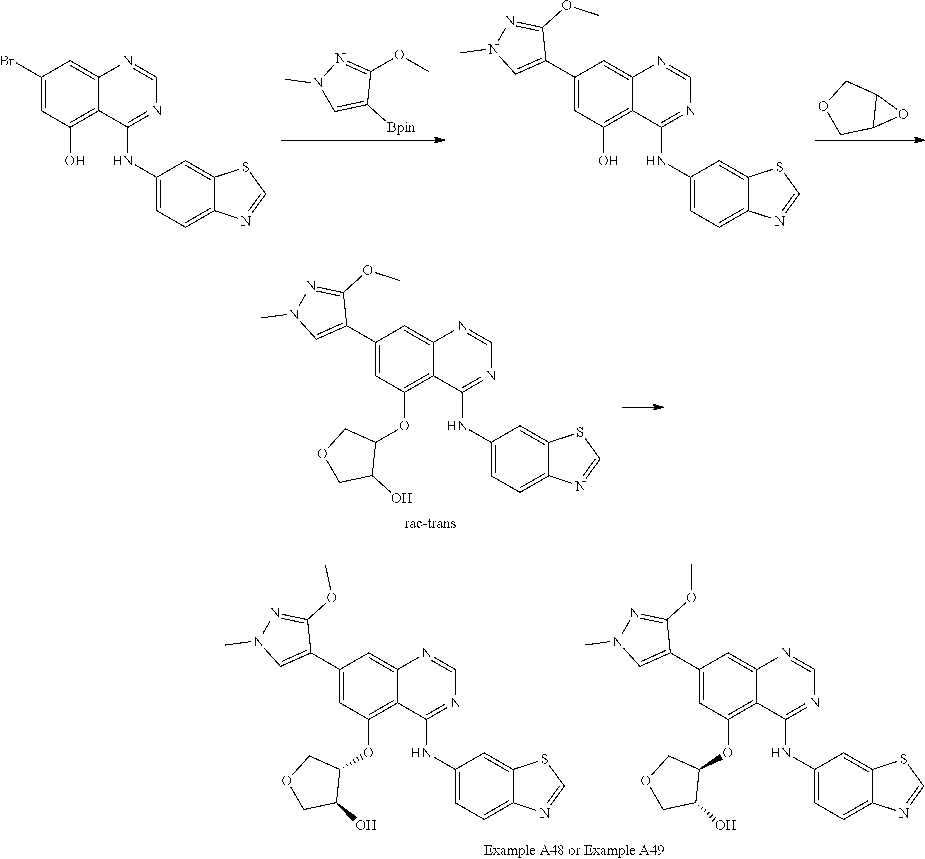

### Step 1: 4-[(1,3-benzothiazol-6-yl)amino]-7-(3-methoxy-1-methyl-1H-pyrazol-4-yl)quinazolin-5-ol

To a solution of 4 Intermediate BP-61 (100 mg, 267 μmol) and 3-methoxy-1-methyl-4-(4,4,5,5-tetramethyl-1,3,2-dioxaborolan-2-yl)-1H-pyrazole (69.7 mg, 293 μmol) in 1,4-dioxane/H2O (3 mL) was added Pd(dppf)Cl₂ (9.65 mg, 13.3 μmol) and K₂CO₃ (73.6 mg, 534 μmol) under N₂. The mixture was stirred at 100° C. for 2 hr. After cooling to rt, the reaction was extracted with DCM and washed with brine. The crude was purified by Prep-TLC (DCM/MeOH=10:1) to afford the title compound (80 mg, 74%) 4 as yellow solid. LC-MS: (ES, m/z): RT=0.559 min, LCMS: m/z=405 [M+1].

### Step 2: 4-((4-(benzo[d]thiazol-6-ylamino)-7-(3-methoxy-1-methyl-1H-pyrazol-4-yl)quinazolin-5-yl)oxy)tetrahydrofuran-3-ol

To 4-[(1,3-benzothiazol-6-yl)amino]-7-(3-methoxy-1-methyl-1H-pyrazol-4-yl)quinazolin-5-ol (100 mg, 247 μmol) and 3,6-dioxabicyclo[3.1.0]hexane (42.5 mg, 494 μmol) in ACN (2 mL) was added NaOH (19.7 mg, 494 μmol) and H₂O (2 mL) under N₂. The mixture was stirred at 80° C. for 2 days. The reaction was extracted with DCM and purified by Prep-TLC (DCM/MeOH=10:1) to afford the title compound (80 mg, 66%) as a yellow solid. LC-MS: (ES, m/z): RT=0.555 min, LCMS: m/z=491 [M+1].

### Step 3: Chiral Separation

4-((4-(benzo[d]thiazol-6-ylamino)-7-(3-methoxy-1-methyl-1H-pyrazol-4-yl)quinazolin-5-yl)oxy)tetrahydrofuran-3-ol (40 mg, 81 μmol) was purified by Prep-chiral-HPLC: Column: CHIRALPAK IH, 2*25 cm, 5 μm; Mobile Phase A: MTBE (0.5% 2M NH₃-MeOH)-HPLC, Mobile Phase B: IPA-HPLC; Flow rate: 20 mL/min; Gradient: 20% B to 20% B in 17 min; Wave Length: 220/254 nm; RT1 (min): 12.242; RT2 (min): 15.972; Sample Solvent: MEOH:DCM=1:2 (0.1% FA); to afford:

Example 48: First Eluting isomer, (3S,4S)-4-((4-(benzo[d]thiazol-6-ylamino)-7-(3-methoxy-1-methyl-1H-pyrazol-4-yl)quinazolin-5-yl)oxy)tetrahydrofuran-3-ol OR (3R,4R)-4-((4-(benzo[d]thiazol-6-ylamino)-7-(3-methoxy-1-methyl-1H-pyrazol-4-yl)quinazolin-5-yl)oxy)tetrahydrofuran-3-ol. (10.3 mg, 25%) as a yellow solid. LC-MS: (ES, m/z): RT=0.809 min, LCMS: m/z=491 [M+1], Chiral-HPLC (ES): RT=3.941, ¹H NMR (300 MHz, DMSO-d6) δ 10.11 (s, 1H), 9.32 (s, 1H), 8.99 (d, J=2.2 Hz, 1H), 8.58 (s, 1H), 8.34 (s, 1H), 8.17-8.06 (m, 1H), 7.74 (dd, J=14.9, 1.7 Hz, 2H), 7.46 (s, 1H), 5.76 (d, J=3.9 Hz, 1H), 5.21 (s, 1H), 4.50 (s, 1H), 4.28-4.12 (m, 3H), 3.99 (s, 3H), 3.79 (s, 3H), 3.68 (d, J=8.2 Hz, 1H), 1.24 (s, 1H),

Example 49: Second Eluting isomer, (3R,4R)-4-((4-(benzo[d]thiazol-6-ylamino)-7-(3-methoxy-1-methyl-1H-pyrazol-4-yl)quinazolin-5-yl)oxy)tetrahydrofuran-3-ol OR (3S,4S)-4-((4-(benzo[d]thiazol-6-ylamino)-7-(3-methoxy-1-methyl-1H-pyrazol-4-yl)quinazolin-5-yl)oxy)tetrahydrofuran-3-ol, (13.5 mg) as a yellow solid. LC-MS: (ES, m/z): RT=0.949 min, LCMS: m/z=491 [M+1], Chiral-HPLC (ES): RT=2.683, ¹H NMR (300 MHz, DMSO-d6) δ 10.12 (s, 1H), 9.32 (s, 1H), 8.99 (s, 1H), 8.58 (s, 1H), 8.34 (s, 1H), 8.11 (d, J=8.8 Hz, 1H), 7.79-7.68 (m, 2H), 7.46 (s, 1H), 5.78 (s, 1H), 5.21 (s, 1H), 4.50 (s, 1H), 4.19 (dd, J=9.8, 6.5 Hz, 3H), 3.99 (s, 3H), 3.79 (s, 3H), 3.68 (d, J=8.4 Hz, 1H), 1.24 (s, 1H),

### Example A50: N-(5-((1R,2S)-2-(dimethylamino)cyclobutoxy)-7-(1-methyl-1H-pyrazol-4-yl)quinazolin-4-yl)benzo[d]thiazol-6-amine

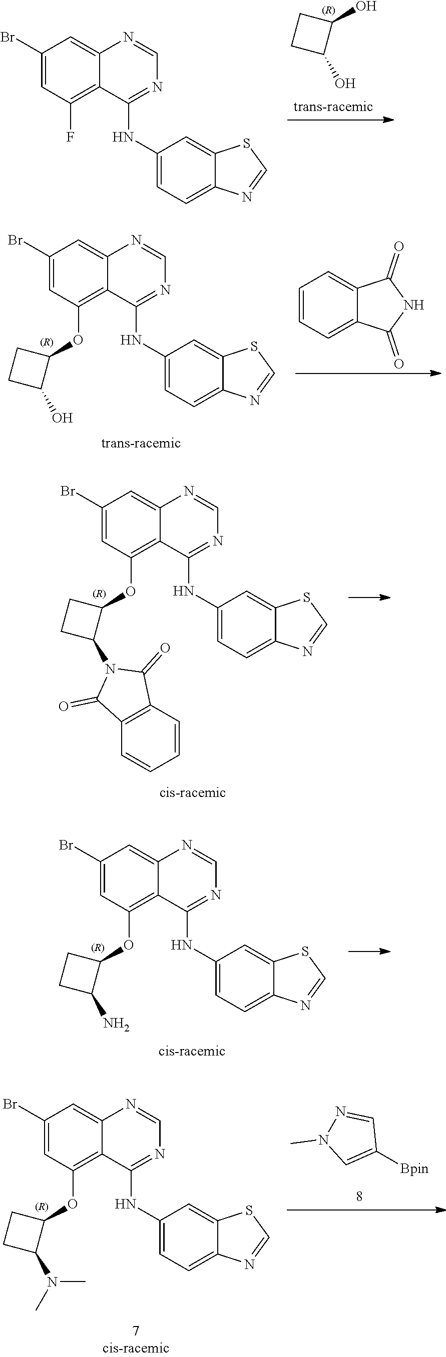

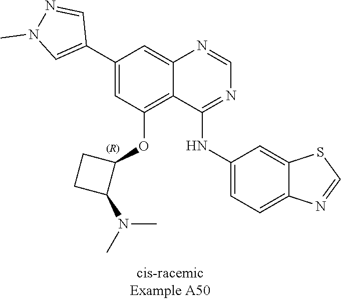

### Step 1: rac-(1R,2R)-2-((4-(benzo[d]thiazol-6-ylamino)-7-bromoquinazolin-5-yl)oxy)cyclobutan-1-ol

t-BuOK (595 mg, 5.32 mmol) was added to Intermediate 1 (1 g, 2.66 mmol) and (1R,2R)-cyclobutane-1,2-diol (234 mg, 2.66 mmol) in THF (60 mL) at rt. The reaction was stirred at 60° C. for 16 h. The mixture was concentrated under vacuum and the residue was purified by a silica gel column with DCM:MeOH (25:1) to afford the title compound (500 mg) LC-MS: (ES, mi/z): RT=0.883 min, mi/z=445 [M+1].

### Step 2: 2-((1S,2R)-2-((4-(benzo[d]thiazol-6-ylamino)-7-bromoquinazolin-5-yl)oxy)cyclobutyl)isoindoline-1,3-dione

Diisopropyl azodicarboxylate (905 mg, 4.48 mmol) was added dropwise to triphenylphosphine (1.46 g, 5.60 mmol), rac-(1R,2R)-2-((4-(benzo[d]thiazol-6-ylamino)-7-bromoquinazolin-5-yl)oxy)cyclobutan-1-ol (500 mg, 1.12 mmol) and 2,3-dihydro-1H-isoindole-1,3-dione (494 mg, 3.36 mmol) in THF (20 mL) at −30° C. The reaction was stirred at rt for 16 h. The resulting mixture was diluted with EA 100 mL and washed with brine 50 mL*2, the organic layer was dried with Na₂SO₄ and concentrated under vacuum. The residue was purified by a prep-TLC with PE:EA=5:1 to afford the title compound (300 mg) as a yellow solid. LC-MS: (ES, m/z): RT=0.983 min, m/z=574 [M+1].

### Step 3: N-(5-((1R,2S)-2-aminocyclobutoxy)-7-bromoquinazolin-4-yl)benzo[d]thiazol-6-amine

N₂H₄ (10 mL 80% aq) was added to 2-((1S,2R)-2-((4-(benzo[d]thiazol-6-ylamino)-7-bromoquinazolin-5-yl)oxy)cyclobutyl)isoindoline-1,3-dione (300 mg, 524 μmol) in EtOH (20 mL) at rt. The resulting mixture was stirred at rt for 16 h. The mixture was diluted with DCM 100 mL and washed with brine 50 mL*2, the organic layer was dried with Na₂SO₄ and concentrated under vacuum. The residue was purified by a prep-TLC with DCM:MeOH=20:1 to afford the title compound (38 mg) as a yellow solid. LC-MS: (ES, m/z): RT=0.757 min, m/z=442 [M+1].

### Step 4: N-(7-bromo-5-((1R,2S)-2-(dimethylamino)cyclobutoxy)quinazolin-4-yl)benzo[d]thiazol-6-amine

STAB (36.2 mg, 171 μmol) was added batch wise to formaldehyde (25.7 mg, 859 μmol) and N-(5-((1R,2S)-2-aminocyclobutoxy)-7-bromoquinazolin-4-yl)benzo[d]thiazol-6-amine (38 mg, 85.9 μmol) in DCM (5 mL) at rt. The resulting mixture was stirred at rt for 4 h. The mixture was concentrated under vacuum and the residue was purified by Prep-TCL with DCM:MeOH=30:1 to 5 afford the title compound, as a white solid. LC-MS: (ES, m/z): RT=1.338 min, m/z=472 [M+1].

### Step 5: N-(5-((1R,2S)-2-(dimethylamino)cyclobutoxy)-7-(1-methyl-1H-pyrazol-4-yl)quinazolin-4-yl)benzo[d]thiazol-6-amine

K₂CO₃ (3.50 mg, 25.4 μmol) was added to Pd(dppf)Cl₂·DCM (3.45 mg, 4.23 μmol), N-(7-bromo-5-((1R,2S)-2-(dimethylamino)cyclobutoxy)quinazolin-4-yl)benzo[d]thiazol-6-amine (10 mg, 21.2 μmol) and 1-methyl-4-(4,4,5,5-tetramethyl-1,3,2-dioxaborolan-2-yl)-1H-pyrazole (6.59 mg, 31.7 μmol) in dioxane/H₂O (1/0.3 mL) at rt. The resulting mixture was stirred at 80° C. for 3 h. The mixture was diluted with EA 10 mL and washed with brine 2 mL*2, the organic layer was dried with Na₂SO₄ and concentrated under vacuum. The residue was purified with Prep-HPLC as following conditions: Column: XBridge Shield RP18 OBD Column, 30*150 mm, 5 μm; Mobile Phase A: Water (10 mmol/L NH₄HCO₃+0.1% NH3·H₂O), Mobile Phase B: ACN; Flow rate: 60 mL/min; Gradient: 50% B to 60% B in 8 min, 60% B; Wave Length: 254; 220 nm to afford the title compound as a yellow solid. LC-MS: (ES, m/z): RT=1.403 min, m/z=472[M+1], 1H NMR (400 MHz, DMSO-d6) δ 10.93 (s, 1H), 9.33 (s, 1H), 8.82 (d, J=2.0 Hz, 1H), 8.56 (s, 1H), 8.43 (s, 1H), 8.12 (d, J=7.5 Hz, 2H), 7.97 (dd, J=8.7, 2.2 Hz, 1H), 7.55 (s, 1H), 7.03 (s, 1H), 5.24 (s, 1H), 3.91 (s, 3H), 3.07 (s, 1H), 2.42-2.06 (m, 9H), 1.90 (s, 1H),

### Example A51: (1S,2R)-2-((4-(benzo[d]thiazol-6-ylamino)-7-(1-methyl-1H-pyrazol-4-yl)quinazolin-5-yl)oxy)cyclobutan-1-ol OR (1R,2S)-2-((4-(benzo[d]thiazol-6-ylamino)-7-(1-methyl-1H-pyrazol-4-yl)quinazolin-5-yl)oxy)cyclobutan-1-ol

### Example A52: (1R,2S)-2-((4-(benzo[d]thiazol-6-ylamino)-7-(1-methyl-1H-pyrazol-4-yl)quinazolin-5-yl)oxy)cyclobutan-1-ol OR (1S,2R)-2-((4-(benzo[d]thiazol-6-ylamino)-7-(1-methyl-1H-pyrazol-4-yl)quinazolin-5-yl)oxy)cyclobutan-1-ol

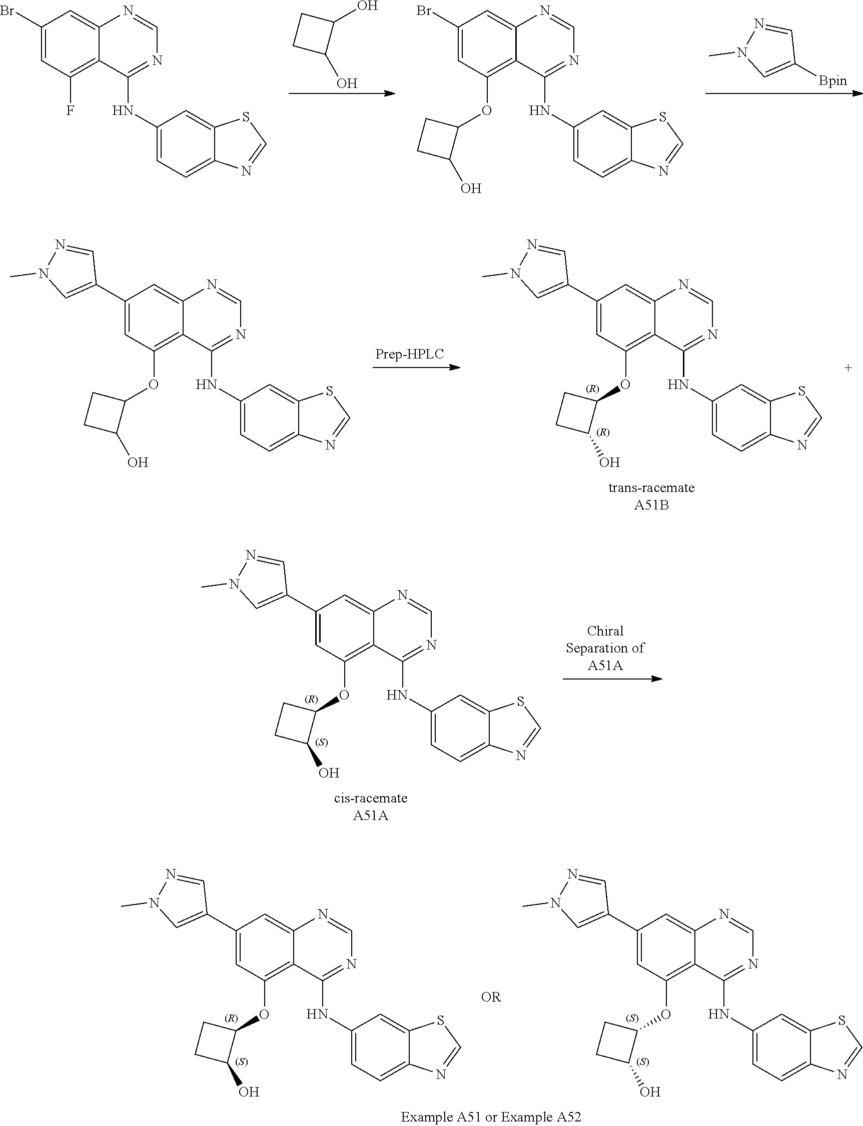

### Step 1: 2-((4-(benzo[d]thiazol-6-ylamino)-7-bromoquinazolin-5-yl)oxy)cyclobutan-1-ol

t-BuOK (297 mg, 2.66 mmol) was added to Intermediate BP-59 (500 mg, 1.33 mmol) and cyclobutane-1,2-diol (234 mg, 2.66 mmol) in THF (20 ml) at rt. The resulting mixture was heated to 100° C. for 16 h. The reaction mixture was diluted with DCM (100 mL), washed sequentially with water (100 mL*3) and saturated brine (100 mL*1). The organic layer was dried over Na₂SO₄, filtered and evaporated to afford crude product. The crude product was purified by prep-TLC with DCM:MeOH=25:1 to afford the title compound (100 mg, 16.9%) as a light yellow solid. LC-MS: (ES, m/z): RT=0.792 min, LCMS: m/z=443 [M+1],

### Step 2: 2-((4-(benzo[d]thiazol-6-ylamino)-7-(1-methyl-1H-pyrazol-4-yl)quinazolin-5-yl)oxy)cyclobutan-1-ol

To a reaction vessel was added: Pd(dppf)Cl₂ (16.4 mg, 22.5 μmol), K₂CO₃ (46.5 mg, 337 μmol), 2-((4-(benzo[d]thiazol-6-ylamino)-7-bromoquinazolin-5-yl)oxy)cyclobutan-1-ol (100 mg, 225 μmol). 1-methyl-4-(4,4,5,5-tetramethyl-1,3,2-dioxaborolan-2-yl)-1H-pyrazole (56.1 mg, 270 μmol) in dioxane/H₂O (6 ml/2 ml) at rt. The reaction was heated to 100° C. for 16 hr then was concentrated under vacuum. The crude product was purified by prep-TLC with DCM:MeOH=20:1 to afford the title compound (70 mg, 70.0%) as an off-white solid. LC-MS: (ES, m/z): RT=0.676 min, LCMS: m/z=445 [M+1].

Step 3: 2-((4-(benzo[d]thiazol-6-ylamino)-7-(1-methyl-1H-pyrazol-4-yl)quinazolin-5-yl)oxy)cyclobutan-1-ol (70 mg) was purified by prep-HPLC: Column: XBridge Prep OBD C18 Column, 30*150 mm, 5 μm; Mobile Phase A: Water (10 mmol/L NH₄HCO₃+0.1% NH₃·H₂O), Mobile Phase B: MeOH-HPLC; Flow rate: 60 mL/min; Gradient: 50% B to 70% B in 8 min; Wave Length: 254; 220 nm to afford

A51A, First eluting isomer: cis-(1S,2R)-2-((4-(benzo[d]thiazol-6-ylamino)-7-(1-methyl-1H-pyrazol-4-yl)quinazolin-5-yl)oxy)cyclobutan-1-ol (21.6 mg), white solid.

A51B, Second eluting isomer: trans-(1R,2R)-2-((4-(benzo[d]thiazol-6-ylamino)-7-(1-methyl-1H-pyrazol-4-yl)quinazolin-5-yl)oxy)cyclobutan-1-ol (36 mg), white solid.

### Step 4: Chiral Separation of Isomer A51A

Isomer A51A (cis-rac) (21.6 mg, 44.9 μmol) in MeOH was Purified by Prep-Chiral-HPLC with following condition: Column: CHIRAL ART Cellulose-SC, 2*25 cm, 5 μm; Mobile Phase A: Hex:DCM=1:1 (0.5% 2M NH₃-MeOH)-HPLC, Mobile Phase B: IPA-HPLC; Flow rate: 20 mL/min; Gradient: 10% B to 10% B in 20 min; Wave Length: 220/254 nm; RT1 (min): 13.118; RT2 (min): 16.344; Sample Solvent: EtOH-HPLC; to afford:

Example A51: (1S,2R)-2-((4-(benzo[d]thiazol-6-ylamino)-7-(1-methyl-1H-pyrazol-4-yl)quinazolin-5-yl)oxy)cyclobutan-1-ol OR (1R,2S)-2-((4-(benzo[d]thiazol-6-ylamino)-7-(1-methyl-1H-pyrazol-4-yl)quinazolin-5-yl)oxy)cyclobutan-1-ol. First eluting isomer, white solid (4 mg, 20.1%) LC-MS: (ES, m/z): RT=1.253 min, LCMS: m/z=445 [M+1], HPLC: RT=3.517 min; 1H NMR (400 MHz, DMSO-d6) δ 10.82 (s, 1H), 9.29 (s, 1H), 8.96 (d, J=1.5 Hz, 1H), 8.55 (s, 1H), 8.42 (s, 1H), 8.09-8.01 (m, 3H), 7.54 (d, J=1.5 Hz, 1H), 7.06 (d, J=1.6 Hz, 1H), 6.12 (d, J=4.9 Hz, 1H), 5.18 (q, J=4.9, 4.4 Hz, 1H), 4.68-4.61 (m, 1H), 3.89 (s, 3H), 2.38 (s, 1H), 2.24 (d, J=7.7 Hz, 1H), 2.16-2.04 (m, 2H), 1.21 (s, 1H),

Example A52: (1R,2S)-2-((4-(benzo[d]thiazol-6-ylamino)-7-(1-methyl-1H-pyrazol-4-yl)quinazolin-5-yl)oxy)cyclobutan-1-ol OR (1S,2R)-2-((4-(benzo[d]thiazol-6-ylamino)-7-(1-methyl-1H-pyrazol-4-yl)quinazolin-5-yl)oxy)cyclobutan-1-ol. Second eluting isomer, white solid (0.7 mg, 3.51%). LC-MS: (ES, m/z): RT=0.797 min, LCMS: m/z=445 [M+1], HPLC: RT=4.356 min; 1H NMR (400 MHz, DMSO-d6) δ 10.82 (s, 1H), 9.29 (s, 1H), 8.96 (d, J=1.4 Hz, 1H), 8.55 (s, 1H), 8.41 (s, 1H), 8.09-8.05 (m, 3H), 7.53 (d, J=1.5 Hz, 1H), 7.06 (d, J=1.6 Hz, 1H), 6.12 (d, J=4.8 Hz, 1H), 5.18 (dt, J=6.0, 3.7 Hz, 1H), 4.64 (t, J=5.1 Hz, 1H), 3.89 (s, 3H), 2.38 (s, 1H), 2.24 (q, J=9.7 Hz, 1H), 2.16-2.03 (m, 2H), 1.21 (s, 1H),

### Example A53: (1R,2R)-2-((4-(benzo[d]thiazol-6-ylamino)-7-(1-methyl-1H-pyrazol-4-yl)quinazolin-5-yl)oxy)cyclobutan-1-ol. OR (1S,2S)-2-((4-(benzo[d]thiazol-6-ylamino)-7-(1-methyl-1H-pyrazol-4-yl)quinazolin-5-yl)oxy)cyclobutan-1-ol

### Example A54: (1S,2S)-2-((4-(benzo[d]thiazol-6-ylamino)-7-(1-methyl-1H-pyrazol-4-yl)quinazolin-5-yl)oxy)cyclobutan-1-ol OR (1R,2R)-2-((4-(benzo[d]thiazol-6-ylamino)-7-(1-methyl-1H-pyrazol-4-yl)quinazolin-5-yl)oxy)cyclobutan-1-ol

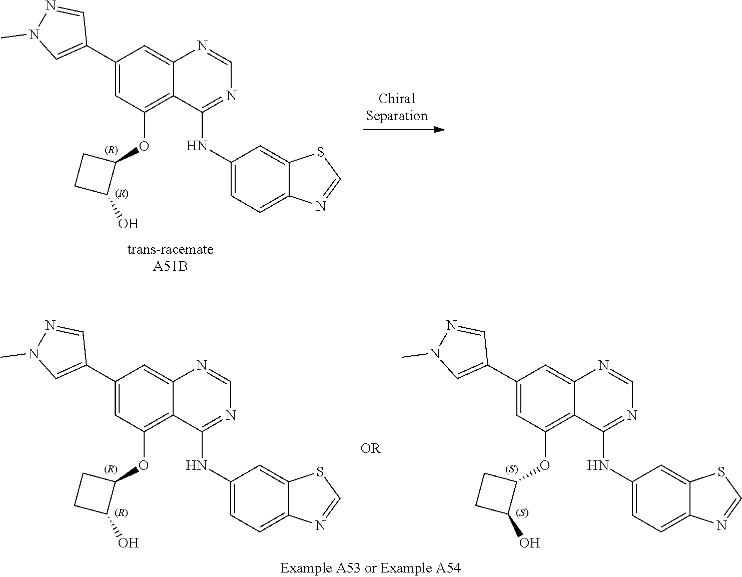

Isomers A51B (trans-ras) were separated by prep-Chiral-HPLC with following condition: Column: CHIRALPAK IH, 2*25 cm, 5 μm; Mobile Phase A: MTBE (0.5% 2M NH₃-MeOH)-HPLC, Mobile Phase B: EtOH-HPLC; Flow rate: 20 mL/min; Gradient: 20% B to 20% B in 11 min; Wave Length: 220/254 nm to afford:

Example A53: First eluting isomer, white solid (11.7 mg, 33.5%) LC-MS: (ES, m/z): RT=1.229 min, LCMS: m/z=445 [M+1], HPLC: RT=1.574 min; 1H NMR (400 MHz, DMSO-d6) δ 10.26 (d, J=3.1 Hz, 1H), 9.30 (d, J=1.0 Hz, 1H), 8.93 (d, J=2.2 Hz, 1H), 8.56 (d, J=1.5 Hz, 1H), 8.38 (d, J=1.6 Hz, 1H), 8.12-8.04 (m, 2H), 7.78 (dt, J=8.8, 1.9 Hz, 1H), 7.56 (d, J=1.5 Hz, 1H), 7.45 (d, J=1.6 Hz, 1H), 6.03 (d, J=6.8 Hz, 1H), 4.78 (q, J=7.6 Hz, 1H), 4.36 (q, J=7.4 Hz, 1H), 3.89 (s, 3H), 2.42 (t, J=9.2 Hz, 1H), 2.20 (q, J=9.4 Hz, 1H), 1.75 (p, J=10.0 Hz, 1H), 1.55 (tt, J=10.6, 8.8 Hz, 1H),

Example A54: Second eluting isomer, white solid (11.7 mg, 32.3%) LC-MS: (ES, m/z): RT=0.786 min, LCMS: m/z=445 [M+1], HPLC: RT=2.442 min; 1H NMR (400 MHz, DMSO-d6) δ 10.26 (d, J=3.2 Hz, 1H), 9.30 (d, J=1.1 Hz, 1H), 8.93 (d, J=2.2 Hz, 1H), 8.56 (d, J=1.7 Hz, 1H), 8.38 (s, 1H), 8.12-8.04 (m, 2H), 7.78 (dt, J=8.9, 1.9 Hz, 1H), 7.56 (d, J=1.5 Hz, 1H), 7.45 (d, J=1.6 Hz, 1H), 6.03 (d, J=6.9 Hz, 1H), 4.78 (q, J=7.5 Hz, 1H), 4.37 (p, J=7.8 Hz, 1H), 3.89 (s, 3H), 2.46-2.37 (m, 1H), 2.20 (q, J=9.4 Hz, 1H), 1.75 (p, J=10.0 Hz, 1H), 1.55 (tt, J=10.4, 8.8 Hz, 1H),

### Example A55: rac-(3R)-3-((4-(benzo[d]thiazol-6-ylamino)-7-(1-methyl-1H-pyrazol-4-yl)quinazolin-5-yl)oxy)-2-((dimethylamino)methyl)butan-1-ol

### Example A56: (R)-2-(1-((4-(benzo[d]thiazol-6-ylamino)-7-(1-methyl-1H-pyrazol-4-yl)quinazolin-5-yl)oxy)ethyl)propane-1,3-diol

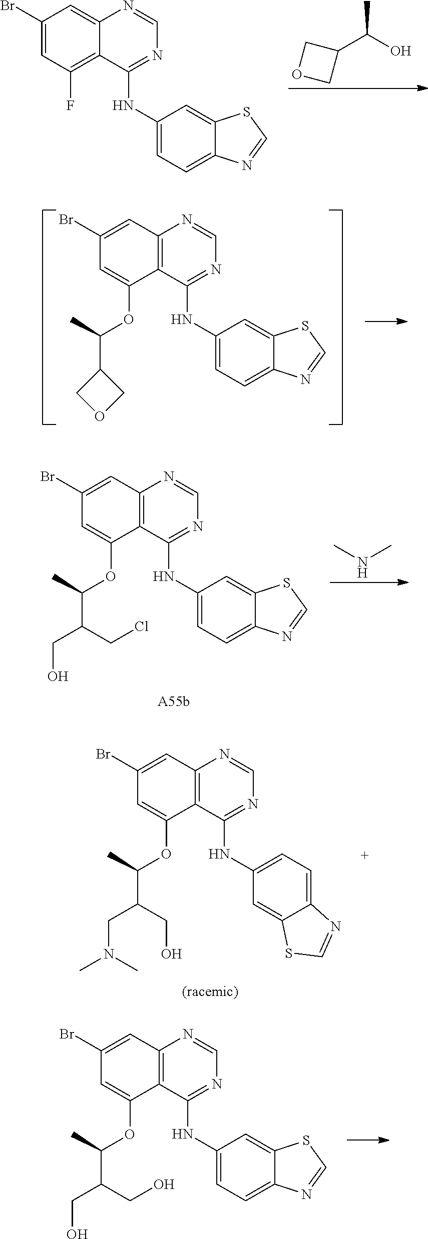

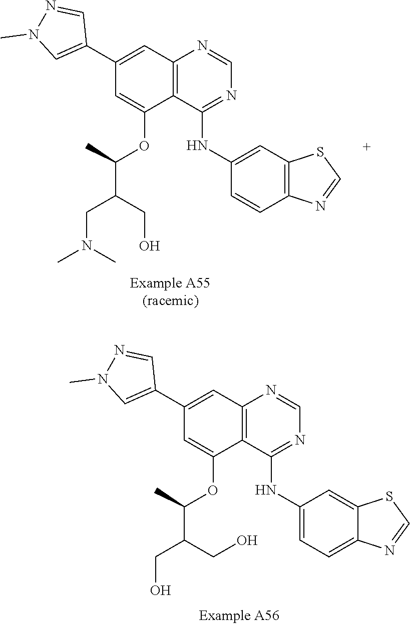 [SMILES: C[C@@H](Oc1cc(-c2cnn(C)c2)cc2ncnc(Nc3ccc4ncsc4c3)c12)C(CO)CN(C)C ; C[C@@H](Oc1cc(-c2cnn(C)c2)cc2ncnc(Nc3ccc4ncsc4c3)c12)C(CO)CO]

### Step 1: (3R)-3-((4-(benzo[d]thiazol-6-ylamino)-7-bromoquinazolin-5-yl)oxy)-2-(chloromethyl)butan-1-ol

To a reaction vessel was added: Intermediate BP-59 (500 mg, 1.333 mmol), (R)-1-(oxetan-3-yl)ethan-1-ol (150 mg, 1.466 mmol), THF (5.33 ml) and potassium t-butoxide (2665 μl, 2.67 mmol). The reaction was stirred at rt for 16 hr. Additional (R)-1-(oxetan-3-yl)ethan-1-ol (150 mg, 1.466 mmol) and Potassium t-butoxide (2.67 ml, 2.67 mmol) was added to the reaction mixture. The reaction was stirred at rt for 4 hr. IN HCl was added and a solid precipitated. The solid was removed by filtration. The filtrate was concentrated and purified by prep-HPLC to obtain A55b, (3R)-3-((4-(benzo[d]thiazol-6-ylamino)-7-bromoquinazolin-5-yl)oxy)-2-(chloromethyl)butan-1-ol (428.2 mg, 0.867 mmol, 65.1% yield) LC-MS: m/z=494 [M+1]

### Step 2: Mixture of (3R)-3-((4-(benzo[d]thiazol-6-ylamino)-7-bromoquinazolin-5-yl)oxy)-2-((dimethylamino)methyl)butan-1-ol and (R)-2-(1-((4-(benzo[d]thiazol-6-ylamino)-7-bromoquinazolin-5-yl)oxy)ethyl)propane-1,3-diol

(3R)-3-((4-(benzo[d]thiazol-6-ylamino)-7-bromoquinazolin-5-yl)oxy)-2-(chloromethyl)butan-1-ol (50 mg, 0.101 mmol), dimethylamine (2000 μl, 4.00 mmol) and THF (2 ml) was heated to 100 C in a sealed MW vial for 16 hr. The reaction was concentrated to afford a mixture that was taken to the next step. rac-(3R)-3-((4-(benzo[d]thiazol-6-ylamino)-7-bromoquinazolin-5-yl)oxy)-2-((dimethylamino)methyl)butan-1-ol. LC-MS: m/z=503 [M+1] and (R)-2-(1-((4-(benzo[d]thiazol-6-ylamino)-7-bromoquinazolin-5-yl)oxy)ethyl)propane-1,3-diol LC-MS: m/z=475 [M+1].

### Step 3: rac-(3R)-3-((4-(benzo[d]thiazol-6-ylamino)-7-(1-methyl-1H-pyrazol-4-yl)quinazolin-5-yl)oxy)-2-((dimethylamino)methyl)butan-1-ol and (R)-2-(1-((4-(benzo[d]thiazol-6-ylamino)-7-(1-methyl-1H-pyrazol-4-yl)quinazolin-5-yl)oxy)ethyl)propane-1,3-diol

To a reaction vessel was added a mixture of rac-(3R)-3-((4-(benzo[d]thiazol-6-ylamino)-7-bromoquinazolin-5-yl)oxy)-2-((dimethylamino)methyl)butan-1-ol and (R)-2-(1-((4-(benzo[d]thiazol-6-ylamino)-7-bromoquinazolin-5-yl)oxy)ethyl)propane-1,3-diol, 1-Methyl-1H-pyrazole-4-boronic acid, pinacol ester (25.3 mg, 0.122 mmol), XantPhos Pd G3 (4.82 mg, 5.08 μmol), potassium phosphate (127 μl, 0.254 mmol) and DMF (1.0 ml). The reaction was sparged with nitrogen. Heated at 90° C. for 16 hr. The reaction was filtered through celite and concentrated. The residue was purified the two products separated by prep-HPLC to afford:

Example A55: rac-(3R)-3-((4-(benzo[d]thiazol-6-ylamino)-7-(1-methyl-1H-pyrazol-4-yl)quinazolin-5-yl)oxy)-2-((dimethylamino)methyl)butan-1-ol (25.4 mg, 0.050 mmol, 49.7% yield). LC-MS: (ES, m/z): RT=2.06 min, LCMS: m/z=504 [M+1]. 1H NMR (MeOD) δ: 8.57 (d, J=2.3 Hz, OH), 8.38 (d, J=2.5 Hz, OH), 8.28 (d, J=2.9 Hz, OH), 8.21-8.13 (m, 1H), 7.63 (d, J=5.2 Hz, OH), 7.50 (d, J=12.7 Hz, OH), 5.44-5.39 (m, OH), 4.23 (d, J=11.6 Hz, OH), 3.94-3.83 (m, OH), 3.71 (dd, J=13.6, 8.3 Hz, OH), 3.33 (s, 4H), 2.53 (dd, J=8.0, 4.3 Hz, OH), 1.69 (dd, J=6.3, 2.1 Hz, 1H)

Example A56: (R)-2-(1-((4-(benzo[d]thiazol-6-ylamino)-7-(1-methyl-1H-pyrazol-4-yl)quinazolin-5-yl)oxy)ethyl)propane-1,3-diol (9.0 mg, 0.019 mmol, 18.61% yield). LC-MS: (ES, m/z): RT=2.343 min, LCMS: m/z=477 [M+1]. 1H NMR (MeOD) δ: 9.33 (s, 1H), 8.56 (d, J=2.1 Hz, 1H), 8.36 (s, 1H), 8.32 (s, 1H), 8.16 (d, J=8.1 Hz, 1H), 8.12 (d, J=2.2 Hz, 1H), 7.71 (d, J=8.8 Hz, 1H), 7.62 (s, 1H), 7.42 (d, J=2.1 Hz, 1H), 5.45 (s, 1H), 4.02 (d, J=2.2 Hz, 3H), 3.96 (d, J=11.0 Hz, 1H), 3.94-3.85 (m, 2H), 2.96 (s, 3H), 2.20 (s, 1H), 1.66 (d, J=6.2 Hz, 3H)

### Example A57: 3-(4-(4-(benzo[d]thiazol-6-ylamino)-5-((tetrahydro-2H-pyran-4-yl)oxy)quinazolin-7-yl)-1H-pyrazol-1-yl)-1,1,1-trifluoropropan-2-ol

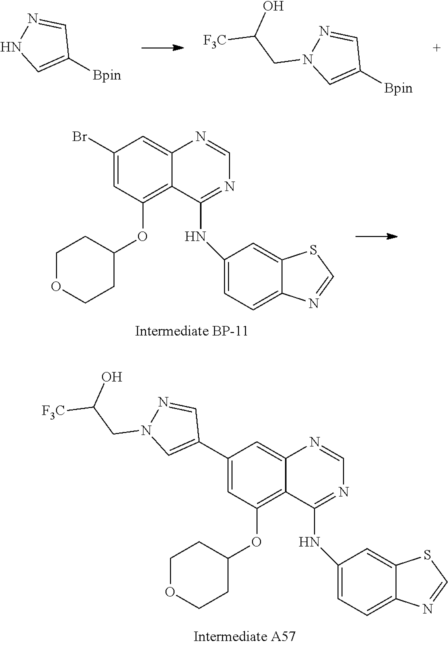

(4,4,5,5-tetramethyl-1,3,2-dioxaborolan-2-yl)-1H-pyrazole (47.7 mg, 0.246 mmol), DMF (2 ml), 2-(trifluoromethyl)oxirane (27.6 mg, 0.246 mmol) and 1,1,1,3,3,3-Hexafluoro-2-propanol (276 mg, 1.640 mmol) were combined in a MW vial and sealed. Heated to 100° C. for 20 min. The reaction was concentrated. To the residue was added XantPhos Pd G3 (7.78 mg, 8.20 μmol), Intermediate BP-11 (75 mg, 0.164 mmol), Potassium phosphate (410 μl, 0.820 mmol) and DMF (3.0 ml). The reaction was heated at 100° C. for 16 hr. Cooled to RT and removed solids by filtration. The filtrate was concentrated then purified by prep-HPLC to afford the title compound (34.3 mg, 0.062 mmol, 37.6% yield). LCMS: m/z=557 [M+1]. 1H NMR (DMSO) δ: 10.84 (s, 1H), 9.44 (s, 1H), 8.84 (s, 1H), 8.70 (d, J=2.3 Hz, 1H), 8.64 (s, 1H), 8.29 (s, 1H), 8.21 (d, J=8.8 Hz, 1H), 7.83 (dd, J=8.8, 2.2 Hz, 1H), 7.64 (s, 1H), 7.55 (s, 1H), 6.84 (s, 1H), 5.30 (tt, J=9.1, 4.3 Hz, 1H), 4.54-4.47 (m, 2H), 4.34 (dd, J=14.6, 9.5 Hz, 1H), 3.97 (dq, J=13.2, 9.3, 6.8 Hz, 2H), 3.62 (t, J=11.1 Hz, 2H), 2.26 (d, J=12.8 Hz, 2H), 2.02 (tt, J=14.4, 7.9 Hz, 2H)

### Example A58: N-(5-((tetrahydro-2H-pyran-4-yl)oxy)-7-(3,3,4-trimethylpiperazin-1-yl)quinazolin-4-yl)benzo[d]thiazol-6-amine

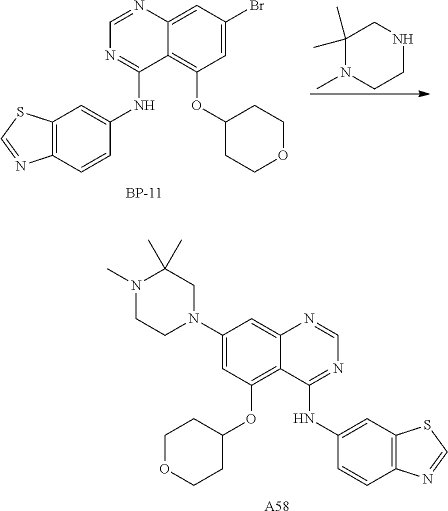

Intermediate BP-11 (128 mg, 0.280 mmol, 1.00 equiv.), 1,2,2-trimethylpiperazine (53.8 mg, 0.420 mmol, 1.50 equiv.), Sphos Pd G3 (12.3 mg, 0.014 mmol, 0.05 equiv.) and t-BuOLi (67.2 mg, 0.84 mmol, 3.00 equiv.) were combined in Dioxane and DMA at rt. The resulting mixture was heated to 100° C. for 16 hrs under N₂. The reaction mixture was diluted with EtOAc (4.00 mL), washed with water (3.00 mL×3). The organic layer was dried over Na₂SO₄, filtered and evaporated to afford crude product. The crude product was purified by prep-HPLC. Fractions were concentrated to afford the title compound (2.47 mg, 0.00490 mmol, 17.5% yield). LC-MS: (ES, m/z): RT=2.057 min, LC-MS: m/z=504.60 [M+1].

### Example A59: N-(5-((1,3-dioxan-5-yl)oxy)-7-(1-methyl-1H-pyrazol-4-yl)quinazolin-4-yl)benzo[d]thiazol-6-amine

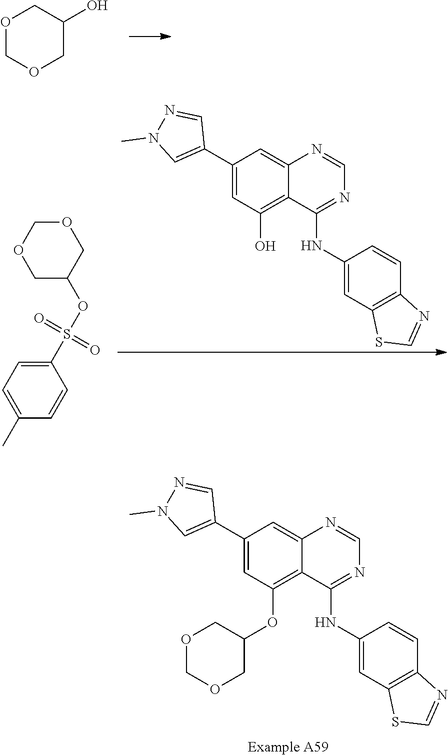 [SMILES: OC1COCOC1 ; Cc1ccc(S(=O)(=O)OC2COCOC2)cc1 ; Cn1cc(-c2cc(O)c3c(Nc4ccc5ncsc5c4)ncnc3c2)cn1 ; Cn1cc(-c2cc(OC3COCOC3)c3c(Nc4ccc5ncsc5c4)ncnc3c2)cn1]

### Step 1: 1,3-dioxan-5-yl 4-methylbenzenesulfonate

1,3-dioxan-5-ol (101 mg, 0.975 mmol, 3 equiv), 4-methylbenzene-1-sulfonyl chloride (185 mg, 0.975 mmol, 3 equiv), and TEA (270 μl, 1.95 mmol, 8 equiv) were combined in DCM (5 mL) and stirred at rt. The resulting mixture was stirred at 30° C. for 16 h. The reaction mixture was concentrated to afford crude product. The crude product was used into the next step without further purification.

### Step 2: N-(5-((1,3-dioxan-5-yl)oxy)-7-(1-methyl-1H-pyrazol-4-yl)quinazolin-4-yl)benzo[d]thiazol-6-amine

Intermediate BP-62 (121 mg, 0.325 mmol, 1 equiv), 1,3-dioxan-5-yl 4-methylbenzenesulfonate (crude, 0.975 mmol, 3 equiv), and Cs₂CO₃ (424 mg, 1.30 mmol, 4 equiv) were combined in DMF (4 mL) at rt. The resulting mixture was heated to 100° C. for 16 h. The reaction mixture was filtered and concentrated. The residue was purified by prep-HPLC. Fractions were concentrated to afford the title compound (17.71 mg, 0.039 mmol, 11.8% yield). LC-MS: (ES, m/z): RT=1.937 min, LC-MS: m/z=461.10 [M+1].

### Example A60: N-(5-(2-(dimethylamino)ethoxy)-7-(1-methyl-1H-pyrazol-4-yl)quinazolin-4-yl)benzo[d]thiazol-6-amine

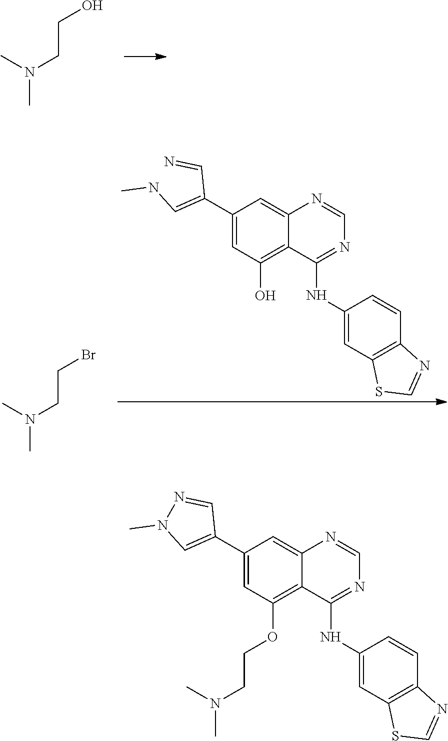

### Step 1: 2-bromo-N,N-dimethylethanamine

2-(dimethylamino)ethanol (86.7 mg, 0.975 mmol, 3 equiv), PS-PPh₃ (1.53 g, 4.59 mmol, 14 equiv, 3 mmol/g) under N₂ atmosphere. The mixture was stirred at 30° C. for 0.5 h. CCl₃Br (964 mg, 4.87 mmol, 15 equiv) was added to the reaction mixture under N₂ atmosphere. The resulting mixture was stirred at 30° C. for 16 h. The reaction mixture was filtered and concentrated to afford crude product which was taken to the next step without further purification.

### Step 2: N-(5-(2-(dimethylamino)ethoxy)-7-(1-methyl-3H-pyrazol-4-yl)quinazolin-4-yl)benzo[d]thiazol-6-amine

Intermediate BP-62: (121 mg, 0.325 mmol, 1 equiv), 2-bromo-N,N-dimethylethanamine (crude, 0.975 mmol, 3 equiv), NaI (292 mg, 1.95 mmol, 6 equiv) and Cs₂CO₃ (424 mg, 1.30 mmol, 4 equiv) were combined in DMF (4 mL) at rt. The resulting mixture was heated to 100° C. for 16 h. The reaction mixture was filtered and concentrated and the residue was purified by prep-HPLC. Fractions were concentrated to afford the title compound (12.74 mg, 0.029 mmol, 8.8% yield). LC-MS: (ES, m/z): RT=2.418 mm, LC-MS: m/z=446.20 [M+1].

TABLE 6

| | | Chemical Name |
|---|---|---|
| # | Structure | Analytical Data |
| 1 | 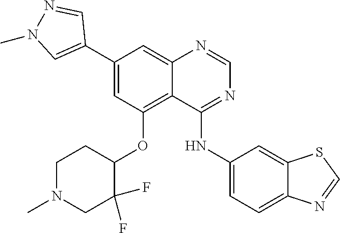 | N-(5-((3,3-difluoro-1-methylpiperidin-4-yl)oxy)-7-(3-methoxy-1- methyl-1H-pyrazol-4-yl)quinazolin-4-yl)benzo[d]thiazol-6-amine LC-MS: (ES, m/z): RT = 1.486 min, LCMS: m/z = 538 [M + 1], ¹H NMR (300 MHz, DMSO-d6) δ 10.11 (s, 1H), 9.33 (s, 1H), 8.95 (d, J = 2.2 Hz, 1H), 8.58 (s, 1H), 8.33 (s, 1H), 8.12 (d, J = 8.8 Hz, 1H), 7.75 (d, J = 1.4 Hz, 1H), 7.66 (dd, J = 8.9, 2.1 Hz, 1H), 7.50 (s, 1H), 5.33 (s, 1H), 3.99 (s, 3H), 3.80 (s, 3H), 2.89 (m, 1H), 2.62 (m, 1H), 2.40 (m, 2H), 2.33 (s, 3H), 2.08-1.95 (m, 1H). |
| 2 | 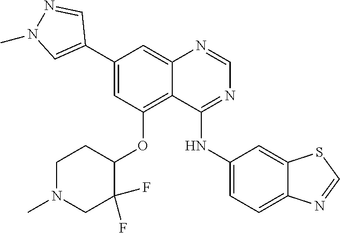 [SMILES: CN1CCC(Oc2cc(-c3cnn(C)c3)cc3ncnc(Nc4ccc5ncsc5c4)c23)C(F)(F)C1] | N-(5-((3,3-difluoro-1-methylpiperidin-4-yl)oxy)-7-(1-methyl-1H- pyrazol-4-yl)quinazolin-4-yl)benzo[d]thiazol-6-amine LC-MS: (ES, m/z): RT = 1.361 min, LCMS: m/z = 508 [M + 1]. ¹H NMR (300 MHz, DMSO-d6) δ 10.16 (s, 1H), 9.33 (s, 1H), 8.95 (d, J = 2.1 Hz, 1H), 8.60 (s, 1H), 8.46 (s, 1H), 8.18 (s, 1H), 8.13 (d, J = 8.8 Hz, 1H), 7.72-7.61 (m, 2H), 7.55 (d, J = 1.5 Hz, 1H), 5.44 (m, 1H), 3.93 (s, 3H), 2.89 (m, 1H), 2.61 (m, 1H), 2.42 (m, 3H), 2.33 (s, 3H), 1.99 (m, 1H). |
| 3 | 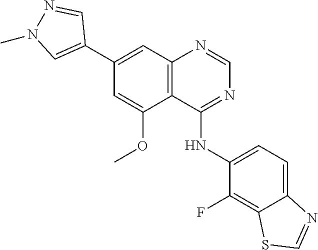 [SMILES: COc1cc(-c2cnn(C)c2)cc2ncnc(Nc3ccc4ncsc4c3F)c12] | 7-fluoro-N-(5-methoxy-7-(1-methyl-1H-pyrazol-4-yl)quinazolin-4- yl)benzo[d]thiazol-6-amine LC-MS: (ES, m/z): RT = 1.299 min, LCMS: m/z = 407.2, [M + 1], ¹H NMR (300 MHz, DMSO-d6) δ 10.05 (s, 1H), 9.43 (d, J = 1.5 Hz, 1H), 8.45 (d, J = 4.8 Hz, 2H), 8.33-8.28 (m, 1H), 8.04 (d, J = 8.4 Hz, 1H), 7.57 (s, 1H), 7.34 (s, 1H), 4.17 (s, 3H), 3.91 (s, 3H). |
| 4 | 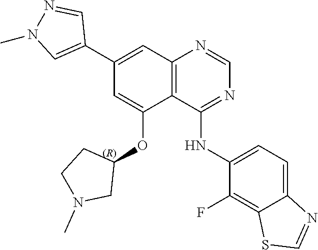 | (R)-7-fluoro-N-(7-(1-methyl-1H-pyrazol-4-yl)-5-((1- methylpyrrolidin-3-yl)oxy)quinazolin-4-yl)benzo[d]thiazol-6-amine LC-MS: (ES, m/z): RT = 2.273 min, LCMS: m/z = 476.3, [M + 1], ¹H NMR (300 MHz, DMSO-d6) δ 10.25 (s, 1H), 9.41 (s, J = 1.2 Hz, 1H), 8.60-8.46 (m, 3H), 8.14 (s, 1H), 8.00 (d, J = 9 Hz, 1H), 7.58 (s, 1H), 7.32 (s, 1H), 5.43 (s, 1H), 3.90 (s, 3H), 2.96-2.77 (m, 3H), 2.44-2.27 (m, 5H), 2.08-2.04 (m, 1H). |
| 5 | 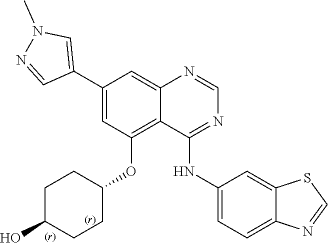 | (1r,4r)-4-((4-(benzo[d]thiazol-6-ylamino)-7-(1-methyl-1H-pyrazol- 4-yl)quinazolin-5-yl)oxy)cyclohexan-1-ol LC-MS: (ES, m/z): RT = 2.273 min, LCMS: m/z = 4732 [M + 1], ¹H NMR (300 MHz, DMSO-d6) δ 10.35 (s, 1H), 9.32 (s, 1H), 8.92 (d, J = 2.3 Hz, 1H), 8.56 (d, J = 1.2 Hz, 1H), 8.49 (s, 1H), 8.17 (s, 1H), 8.12 (d, J = 8.8 Hz, 1H), 7.75 (dd, J = 8.8, 2.3 Hz, 1H), 7.56 (d, J = 1.5 Hz, 1H), 7.41 (d, J = 1.4 Hz, 1H), 4.96 (s, 1H), 4.70 (d, J = 4.1 Hz, 1H), 3.92 (s, 3H), 3.65 (s, 1H), 2.28 (s, 2H), 2.08 (s, 1H), 1.94 (d, J = 12.6 Hz, 2H), 1.81-1.68 (m, 2H), 1.51 (q, J = 10.4 Hz, 2H). |
| 6 7 | 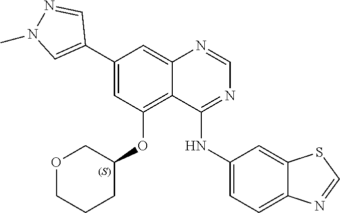 or 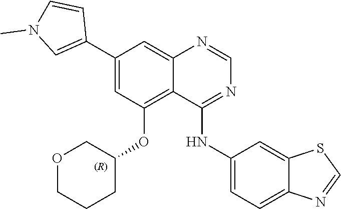 | (R)-N-(7-(1-methyl-1H-pyrazol-4-yl)-5-((tetrahydro-2H-pyran-3- yl)oxy)quinazolin-4-yl)benzo[d]thiazol-6-amine OR (S)-N-(7-(1- methyl-1H-pyrazol-4-yl)-5-((tetrahydro-2H-pyran-3- yl)oxy)quinazolin-4-yl)benzo[d]thiazol-6-amine First Eluting Isomer: LC-MS: (ES, m/z): RT = 2.415 min, LCMS: m/z = 459 [M + 1], ¹H NMR (300 MHz, DMSO-d6) δ 10.69 (s, 1H), 9.31 (s, 1H), 9.09 (d, J = 2.2 Hz, 1H), 8.61 (s, 1H), 8.47 (s, 1H), 8.16 (d, J = 0.8 Hz, 1H), 8.10 (d, J = 8.9 Hz, 1H), 7.94 (dd, J = 8.9, 2.1 Hz, 1H), 7.59 (d, J = 1.4 Hz, 1H), 7.43 (s, 1H), 5.17 (s, 1H), 4.20 (d, J = 12.3 Hz, 1H), 3.93 (t, J = 13.2 Hz, 2H), 3.92 (s, 3H), 3.65 (t, J = 10.4 Hz, 1H), 2.20 (d, J = 13.8 Hz, 1H), 1.94 (d, J = 12.9 Hz, 1H), 1.82 (d, J = 12.9 Hz, 1H), 1.51 (d, J = 13.7 Hz, 1H), 1.15 (s, 1H). Second Eluting Isomer: LC-MS: (ES, m/z): RT = 1.357 min, LCMS: m/z = 459 [M + 1], ¹H NMR (300 MHz, DMSO-d6) δ 10.69 (s, 1H), 9.31 (s, 1H), 9.09 (d, J = 2.2 Hz, 1H), 8.61 (s, 1H), 8.47 (s, 1H), 8.16 (d, J = 0.8 Hz, 1H), 8.10 (d, J = 8.9 Hz, 1H), 7.94 (dd, J = 8.9, 2.1 Hz, 1H), 7.59 (d, J = 1.4 Hz, 1H), 7.43 (s, 1H), 5.17 (s, 1H), 4.20 (d, J = 12.3 Hz, 1H), 3.93 (t, J = 13.2 Hz, 2H), 3.92 (s, 3H), 3.65 (t, J = 10.4 Hz, 1H), 2.20 (d, J = 13.9 Hz, 1H), 1.94 (d, J = 12.8 Hz, 1H), 1.82 (d, J = 13.0 Hz, 1H), 1.51 (d, J = 13.9 Hz, 1H), 1.15 (s, 1H). |
| 8 | 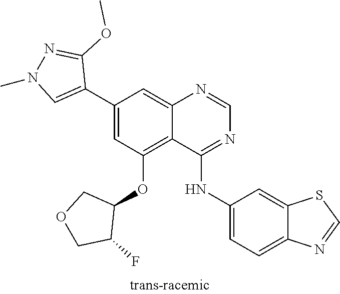 [SMILES: COc1nn(C)cc1-c1cc(O[C@@H]2COC[C@H]2F)c2c(Nc3ccc4ncsc4c3)ncnc2c1] | trans-N-(5-((-4-fluorotetrahydrofuran-3-yl)oxy)-7-(3-methoxy-1- methyl-1H-pyrazol-4-yl)quinazolin-4-yl)benzo[d]thiazol-6-amine LC-MS: (ES, m/z): RT = 1.557 min, LCMS: m/z = 493 [M + 1], ¹H NMR (300 MHz, DMSO-d6) δ 9.88 (s, 1H), 9.32 (s, 1H), 8.93 (d, J = 2.1 Hz, 1H), 8.58 (s, 1H), 8.37 (s, 1H), 8.10 (d, J = 8.8 Hz, 1H), 7.78 (dd, J = 8.8, 2.2 Hz, 1H), 7.72 (d, J = 1.4 Hz, 1H), 7.53 (d, J = 1.6 Hz, 1H), 5.78-5.52 (m, 2H), 4.37-4.01 (m, 4H), 3.98 (s, 3H), 3.79 (s, 3H). |
| 9 | 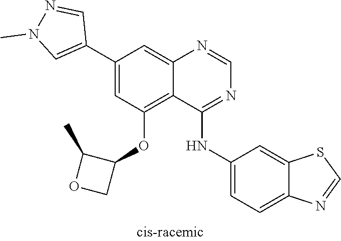 [SMILES: C[C@@H]1OC[C@@H]1Oc1cc(-c2cnn(C)c2)cc2ncnc(Nc3ccc4ncsc4c3)c12] | cis-N-(7-(1-methyl-1H-pyrazol-4-yl)-5-((-2-methyloxetan-3- yl)oxy)quinazolin-4-yl)benzo[d]thiazol-6-amine LC-MS: (ES, m/z): RT = 5.409 min, LCMS: m/z = 445 [M + 1], ¹H NMR (300 MHz, DMSO-d6) δ 9.98 (s, 1H), 9.34 (s, 1H), 8.82 (d, J = 2.2 Hz, 1H), 8.56 (s, 1H), 8.47 (s, 1H), 8.19-8.07 (m, 2H), 7.85 (dd, J = 8.8, 2.2 Hz, 1H), 7.61 (d, J = 1.4 Hz, 1H), 6.95 (d, J = 1.6 Hz, 1H), 5.75 (q, J = 5.9 Hz, 1H), 5.36 (p, J = 6.3 Hz, 1H), 5.07 (dd, J = 7.7, 6.2 Hz, 1H), 4.80 (dd, J = 7.7, 5.2 Hz, 1H), 3.91 (s, 3H), 1.44 (d, J = 6.4 Hz, 3H). |
| 10 | 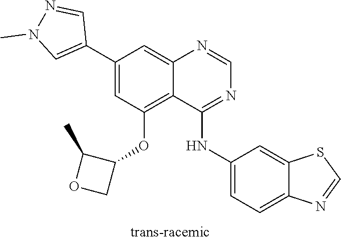 [SMILES: C[C@@H]1OC[C@H]1Oc1cc(-c2cnn(C)c2)cc2ncnc(Nc3ccc4ncsc4c3)c12] | trans-N-(7-(1-methyl-1H-pyrazol-4-yl)-5-(((2S,3S)-2-methyloxetan- 3-yl)oxy)quinazolin-4-yl)benzo[d]thiazol-6-amine LC-MS: (ES, m/z): RT = 1.072 min, LCMS: m/z = 445 [M + 1], ¹H NMR (400 MHz, DMSO-d6) δ 10.09 (s, 1H), 9.33 (s, 1H), 8.92 (d, J = 2.1 Hz, 1H), 8.59 (s, 1H), 8.44 (s, 1H), 8.12 (d, J = 8.4 Hz, 2H), 7.85 (dd, J = 8.8, 2.2 Hz, 1H), 7.61 (d, J = 1.5 Hz, 1H), 6.93 (d, J = 1.5 Hz, 1H), 5.40 (q, J = 5.3 Hz, 1H), 5.23-5.16 (m, 1H), 5.03 (t, J = 6.6 Hz, 1H), 4.65-4.57 (m, 1H), 3.92 (s, 3H), 1.60 (d, J = 6.3 Hz, 3H). |
| 11 | 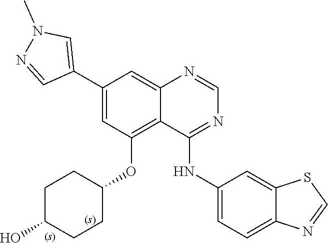 [SMILES: Cn1cc(-c2cc(O[C@H]3CC[C@@H](O)CC3)c3c(Nc4ccc5ncsc5c4)ncnc3c2)cn1] | (1s,4s)-4-((4-(benzo[d]thiazol-6-ylamino)-7-(1-methyl-1H-pyrazol- 4-yl)quinazolin-5-yl)oxy)cyclohexan-1-ol LC-MS: (ES, m/z): RT = 2.023 min, LCMS: m/z = 473 [M + 1], ¹H NMR (300 MHz, DMSO-d6) δ 10.34 (s, 1H), 9.33 (s, 1H), 8.90 (d, J = 2.2 Hz, 1H), 8.55 (s, 1H), 8.47 (s, 1H), 8.18-8.08 (m, 2H), 7.74 (dd, J = 8.9, 2.2 Hz, 1H), 7.56 (d, J = 1.4 Hz, 1H), 7.40 (s, 1H), 5.06 (s, 1H), 4.65 (d, J = 3.7 Hz, 1H), 3.92 (s, 3H), 3.77 (s, 1H), 2.10 (d, J = 14.0 Hz, 2H), 1.95 (s, 2H), 1.72 (s, 4H). |
| 12 | 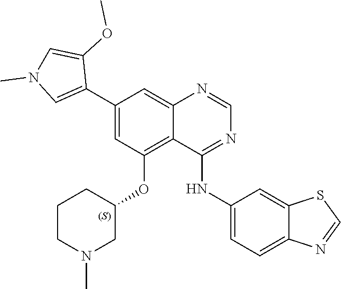 | (S)-N-(7-(3-methoxy-1-methyl-1H-pyrazol-4-yl)-5-((1- methylpiperidin-3-yl)oxy)quinazolin-4-yl)benzo[d]thiazol-6-amine LC-MS: (ES, m/z): RT = 1.787 min, LCMS: m/z = 502 [M + 1], ¹H NMR (400 MHz, DMSO-d6) ¹H δ 10.86 (s, 1H), 9.31 (s, 1H), 8.93 (d, J = 2.1 Hz, 1H), 8.54 (s, 1H), 8.35 (s, 1H), 8.11 (d, J = 8.8 Hz, 1H), 8.00 (dd, J = 8.9, 2.1 Hz, 1H), 7.65 (d, J = 1.4 Hz, 1H), 7.38 (d, J = 1.6 Hz, 1H), 5.17 (s, 1H), 3.98 (s, 3H), 3.77 (s, 3H), 3.18 (d, J = 12.1 Hz, 1H), 2.86 (d, J = 11.2 Hz, 1H), 2.42 (d, J = 12.1 Hz, 1H), 2.32 (s, 3H), 2.11 (t, J = 10.3 Hz, 2H), 1.74 (t, J = 12.2 Hz, 1H), 1.67- 1.45 (m, 2H). |
| 13 | 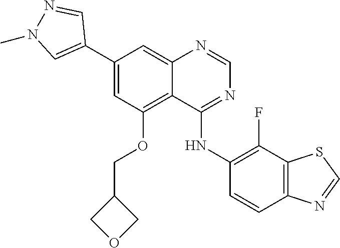 [SMILES: Cn1cc(-c2cc(OCC3COC3)c3c(Nc4ccc5ncsc5c4F)ncnc3c2)cn1] | 7-fluoro-N-(7-(1-methyl-1H-pyrazol-4-yl)-5-(oxetan-3- ylmethoxy)quinazolin-4-yl)benzo[d]thiazol-6-amine LC-MS: (ES, m/z): RT = 1.183 min, LCMS: m/z = 463 [M + 1], ¹H NMR (300 MHz, DMSO-d6) δ 10.04 (s, 1H), 9.45 (s, 1H), 8.49 (d, J = 7.8 Hz, 2H), 8.25 (s, 1H), 8.19 (s, 1H), 8.00 (d, J = 9.1 Hz, 1H), 7.62 (s, 1H), 7.44 (s, 1H),4.90 (t, J = 6.8 Hz, 2H), 4.65 (d, J = 5.1 Hz, 2H), 4.57 (t, J = 5.6 Hz, 2H), 3.93 (s, 3H),3.50 (s, 1H). |
| 14 | 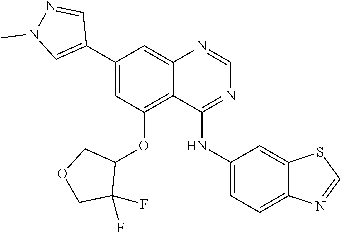 [SMILES: Cn1cc(-c2cc(OC3COCC3(F)F)c3c(Nc4ccc5ncsc5c4)ncnc3c2)cn1] | N-(5-((4,4-difluorotetrahydrofuran-3-yl)oxy)-7-(1-methyl-1H- pyrazol-4-yl)quinazolin-4-yl)benzo[d]thiazol-6-amine LC-MS: (ES, m/z): RT = 1.540 min, LCMS: m/z = 481 [M + 1], ¹H NMR (300 MHz, DMSO-d6) δ 9.94 (s, 1H), 9.33 (s, 1H), 8.92 (d, J = 2.1 Hz, 1H), 8.61 (s, 1H), 8.48 (s, 1H), 8.17 (d, J = 0.7 Hz, 1H), 8.11 (d, J = 8.8 Hz, 1H), 7.72 (dd, J = 8.9, 2.2 Hz, 1H), 7.66 (d, J = 1.4 Hz, 1H), 7.39 (d, J = 1.5 Hz, 1H), 5.76 (d, J = 13.8 Hz, 1H), 4.67 (dd, J = 10.7, 5.4 Hz, 1H), 4.36-4.16 (m, 3H), 3.92 (s, 3H). |
| 15 | 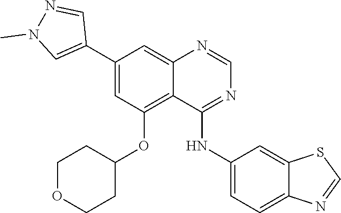 [SMILES: Cn1cc(-c2cc(OC3CCOCC3)c3c(Nc4ccc5ncsc5c4)ncnc3c2)cn1] | N-(7-(1-methyl-1H-pyrazol-4-yl)-5-((tetrahydro-2H-pyran-4- yl)oxy)quinazolin-4-yl)benzo[d]thiazol-6-amine LC-MS: (ES, m/z): RT = 1.299 min, LCMS: m/z = 459 [M + 1]. ¹H NMR (400 MHz, DMSO-d6) 1H δ 10.30 (s, 1H), 9.32 (s, 1H), 8.91 (d, J = 2.2 Hz, 1H), 8.57 (s, 1H), 8.47 (s, 1H), 8.17 (s, 1H), 8.12 (d, J = 8.8 Hz, 1H), 7.78 (dd, J = 8.9, 2.2 Hz, 1H), 7.58 (d, J = 1.4 Hz, 1H), 7.44 (d, J = 1.6 Hz, 1H), 5.19 (dq, J = 9.3, 4.5 Hz, 1H), 3.98 (dt, J = 11.7, 4.0 Hz, 2H), 3.92 (s, 3H), 3.62 (td, J = 11.8, 11.1, 2.4 Hz, 2H), 2.29 (d, J = 12.6 Hz, 2H), 2.02-1.86 (m, 2H). |
| 16 | 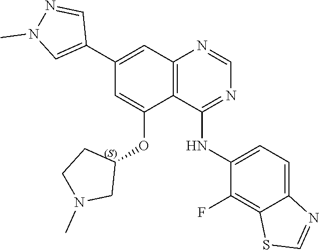 | (S)-7-fluoro-N-(7-(1-methyl-1H-pyrazol-4-yl)-5-((1- methylpyrrolidin-3-yl)oxy)quinazolin-4-yl)benzo[d]thiazol-6-amine LC-MS: (ES, m/z): RT = 1.387 min, LCMS: m/z = 476.3, [M + 1], 1H NMR (300 MHz, DMSO-d6) δ 10.25 (s, 1H), 9.42 (s, 1H), 9.41- 8.47 (m, 3H), 8.15 (s, 1H), 8.01 (d, J = 9 Hz, 1H), 7.59 (s, 1H), 7.33 (s, 1H), 5.44 (s, 1H), 3.90 (s, 3H), 2.96-2.80 (m, 3H), 2.49-2.27 (m, 6H). |
| 17 | 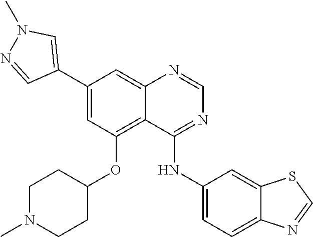 [SMILES: CN1CCC(Oc2cc(-c3cnn(C)c3)cc3ncnc(Nc4ccc5ncsc5c4)c23)CC1] | N-(7-(1-methyl-1H-pyrazol-4-yl)-5-((1-methylpiperidin-4- yl)oxy)quinazolin-4-yl)benzo[d]thiazol-6-amine LC-MS: (ES, m/z): RT = 2.050 min, LCMS: m/z = 472 [M + 1], ¹H NMR (300 MHz, DMSO-d6) δ 10.31 (s, 1H), 9.33 (s, 1H), 8.91 (d, J = 2.1 Hz, 1H), 8.56 (s, 1H), 8.48 (s, 1H), 8.20-8.08 (m, 2H), 7.75 (dd, J = 8.8, 2.2 Hz, 1H), 7.57 (d, J = 1.4 Hz, 1H), 7.41 (d, J = 1.6 Hz, 1H), 5.02 (d, J = 4.2 Hz, 1H), 3.92 (s, 3H), 2.70 (d, J = 11.4 Hz, 2H), 2.20 (s, 7H), 1.96 (d, J = 9.6 Hz, 2H). |
| 18 | 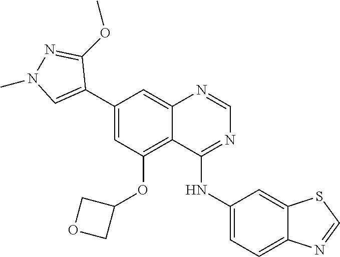 [SMILES: COc1nn(C)cc1-c1cc(OC2COC2)c2c(Nc3ccc4ncsc4c3)ncnc2c1] | N-(7-(3-methoxy-1-methyl-1H-pyrazol-4-yl)-5-(oxetan-3- yloxy)quinazolin-4-yl)benzo[d]thiazol-6-amine LC-MS: (ES, m/z): RT = 1.063 min, LCMS: m/z = 461 [M + 1], ¹H NMR (300 MHz, Methanol-d4) δ 9.24 (s, 1H), 8.58 (s, 1H), 8.12 (d, J = 8.8 Hz, 1H), 8.05 (d, J = 2.2 Hz, 1H), 7.78 (s, 1H), 7.72 (d, J = 1.7 Hz, 1H), 7.48 (dd, J = 8.8, 2.2 Hz, 1H), 7.28 (d, J = 1.6 Hz, 1H), 4.88 (s, 1H), 4.38 (dd, J = 14.5, 2.3 Hz, 1H), 4.18 (dd, J = 14.5, 8.6 Hz, 1H), 3.99-3.82 (m, 2H), 3.71 (d, J = 3.6 Hz, 6H). |
| 19 | 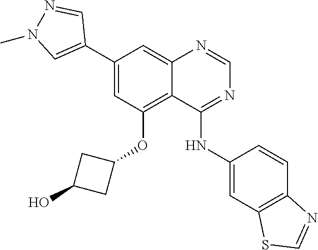 [SMILES: Cn1cc(-c2cc(O[C@H]3C[C@H](O)C3)c3c(Nc4ccc5ncsc5c4)ncnc3c2)cn1] | trans-3-((4-(benzo[d]thiazol-6-ylamino)-7-(1-methyl-1H-pyrazol-4- yl)quinazolin-5-yl)oxy)cyclobutan-1-ol LC-MS: (ES, m/z): RT = 1.310 min, m/z = 445 [M + 1], ¹H NMR (400 MHz, DMSO-d6) δ 10.26 (s, 1H), 9.32 (s, 1H), 8.95 (d, J = 2.1 Hz, 1H), 8.59 (s, 1H), 8.46 (s, 1H), 8.12 (dd, J = 4.8, 4.0 Hz, 2H), 7.78 (dd, J = 8.8, 2.2 Hz, 1H), 7.57 (d, J = 1.5 Hz, 1H), 7.05 (d, J = 1.6 Hz, 1H), 5.39-5.29 (m, 2H), 4.56 (td, J = 6.8, 3.3 Hz, 1H), 3.91 (s, 3H), 2.69-2.52 (m, 3H). |
| 20 | 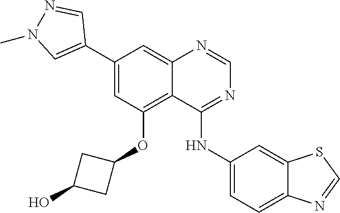 [SMILES: Cn1cc(-c2cc(O[C@H]3C[C@@H](O)C3)c3c(Nc4ccc5ncsc5c4)ncnc3c2)cn1] | cis-3-((4-(benzo[d]thiazol-6-ylamino)-7-(1-methyl-1H-pyrazol-4- yl)quinazolin-5-yl)oxy)cyclobutan-1-ol LC-MS: (ES, m/z): RT = 1.209 min, LCMS: m/z = 445 [M + 1], ¹H NMR (400 MHz, DMSO-d6) δ 10.30 (s, 1H), 9.31 (s, 1H), 8.95 (d, J = 2.1 Hz, 1H), 8.57 (s, 1H), 8.44 (s, 1H), 8.15-8.08 (m, 2H), 7.74 (dd, J = 8.8, 2.2 Hz, 1H), 7.55 (d, J = 1.5 Hz, 1H), 7.10 (d, J = 1.6 Hz, 1H), 5.35 (d, J = 6.9 Hz, 1H), 4.75 (p, J = 6.8 Hz, 1H), 3.97 (d, J = 7.0 Hz, 1H), 3.89 (s, 3H), 3.10 (m, 2H), 2.20 (m, 2H). |
| 21 | 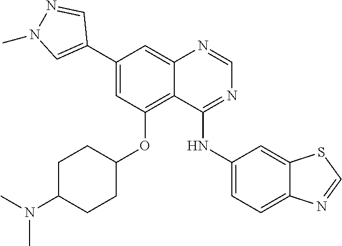 [SMILES: CN(C)C1CCC(Oc2cc(-c3cnn(C)c3)cc3ncnc(Nc4ccc5ncsc5c4)c23)CC1] | N-(5-((4-(dimethylamino)cyclohexyl)oxy)-7-(1-methyl-1H-pyrazol- 4-yl)quinazolin-4-yl)benzo[d]thiazol-6-amine LC-MS: (ES, m/z): RT = 1.508 min, LCMS: m/z = 500 [M + 1], ¹H NMR (300 MHz, DMSO-d6) δ 10.38 (s, 0H), 9.31 (s, 1H), 8.94 (t, J = 2.5 Hz, 1H), 8.56 (s, 1H), 8.46 (s, 1H), 8.19-8.06 (m, 2H), 7.74 (dd, J = 8.9, 2.2 Hz, 1H), 7.55 (s, 1H), 7.38 (s, 1H), 4.88 (s, 1H), 3.92 (s, 3H), 2.43-2.33 (m, 3H), 2.23 (s, 6H), 1.93 (d, J = 12.2 Hz, 2H), 1.75 (d, J = 12.2 Hz, 1H), 1.67 (d, J = 11.7 Hz, 1H), 1.52 (q, J = 12.4 Hz, 2H) |
| 22 | 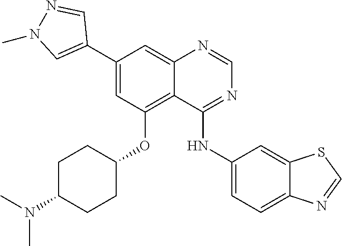 [SMILES: Cn1cc(-c2cc(O[C@H]3CC[C@@H](N(C)C)CC3)c3c(Nc4ccc5ncsc5c4)ncnc3c2)cn1] | N-(5-(((1s,4s)-4-(dimethylamino)cyclohexyl)oxy)-7-(1-methyl-1H- pyrazol-4-yl)quinazolin-4-yl)benzo[d]thiazol-6-amine LC-MS: (ES, m/z): RT = 1.319 min, LCMS: m/z = 500 [M + 1], ¹H NMR (300 MHz, DMSO-d6) δ 10.23 (s, 1H), 9.34 (s, 1H), 8.80 (d, J = 2.1 Hz, 1H), 8.50 (d, J = 16.4 Hz, 2H), 8.19-8.07 (m, 2H), 7.84 (dd, J = 8.8, 2.2 Hz, 1H), 7.57 (d, J = 1.4 Hz, 1H), 7.43 (d, J = 1.6 Hz, 1H), 5.22 (s, 1H), 3.92 (s, 3H), 2.23 (d, J = 11.8 Hz, 3H), 2.07 (s, 6H), 1.79 (dd, J = 27.3, 13.4 Hz, 4H), 1.59 (d, J = 11.7 Hz, 2H). |
| 23 24 | 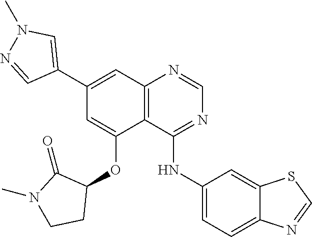 [SMILES: CN1CC[C@H](Oc2cc(-c3cnn(C)c3)cc3ncnc(Nc4ccc5ncsc5c4)c23)C1=O] or 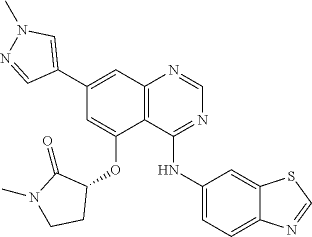 [SMILES: CN1CC[C@@H](Oc2cc(-c3cnn(C)c3)cc3ncnc(Nc4ccc5ncsc5c4)c23)C1=O] | (R)-3-((4-(benzo[d]thiazol-6-ylamino)-7-(1-methyl-1H-pyrazol-4- yl)quinazolin-5-yl)oxy)-1-methylpyrrolidin-2-one OR (S)-3-((4- (benzo[d]thiazol-6-ylamino)-7-(1-methyl-1H-pyrazol-4- yl)quinazolin-5-yl)oxy)-1-methylpyrrolidin-2-one First Eluting Isomer: LC-MS: (ES, m/z): RT = 1.200 min, LCMS: m/z = 472 [M + 1], Chiral-HPLC (ES): RT = 1.510, ¹H NMR (300 MHz,DMSO-d6) δ 10.51 (s, 1H), 9.29 (s, 1H), 9.04 (d, J = 2.1 Hz, 1H), 8.57 (d, J = 1.3 Hz, 1H), 8.45 (s, 1H), 8.14 (s, 1H), 8.08 (d, J = 8.8 Hz, 1H), 7.91 (dd, J = 8.8, 2.2 Hz, 1H), 7.60 (d, J = 1.4 Hz, 1H), 7.30 (d, J = 1.5 Hz, 1H), 5.37 (t, J = 8.4 Hz, 1H), 3.90 (s, 3H), 3.49 (dd, J = 9.1, 4.1 Hz, 2H), 3.10 (td, J = 8.3, 4.3 Hz, 1H), 2.93 (s, 3H), 2.15 (dd, J = 12.4, 9.0 Hz, 1H) Second Eluting Isomer: LC-MS: (ES, m/z): RT = 1.197 min, LCMS: m/z = 472 [M + 1], Chiral-HPLC (ES): RT = 2.536, ¹H NMR (300 MHz, DMSO-d6) δ 10.51 (s, 1H), 9.29 (s, 1H), 9.03 (t, J = 3.0 Hz, 1H), 8.57 (d, J = 1.2 Hz, 1H), 8.45 (s, 1H), 8.14 (s, 1H), 8.08 (d, J = 8.9 Hz, 1H), 7.91 (dd, J = 8.8, 2.2 Hz, 1H), 7.60 (d, J = 1.4 Hz, 1H), 7.30 (d, J = 1.5 Hz, 1H), 5.37 (t, J = 8.4 Hz, 1H), 3.90 (s, 3H), 3.49 (dd, J = 9.1, 4.1 Hz, 2H), 3.10 (td, J = 8.3, 4.1 Hz, 1H), 2.93 (s, 3H), 2.15 (dd, J = 12.5, 8.9 Hz, 1H) |
| 25 | 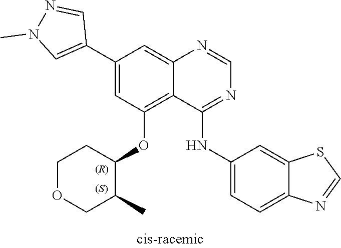 | cis-racemic N-(7-(1-methyl-1H-pyrazol-4-yl)-5-(((3S,4R)-3- methyltetrahydro-2H-pyran-4-yl)oxy)quinazolin-4- yl)benzo[d]thiazol-6-amine LC-MS: (ES, m/z): RT = 1.380 min, LCMS: m/z = 473 [M + 1]. ¹H NMR (300 MHz,DMSO-d6) & 10.13 (s, 1H), 9.33 (s, 1H), 8.83 (d, J = 2.1 Hz, 1H), 8.51 (d, J = 22.8 Hz, 2H), 8.27-8.03 (m, 2H), 7.85- 7.70 (m, 1H), 7.58 (s, 1H), 7.46 (s, 1H), 5.32 (s, 1H), 3.92 (s, 4H), 3.77 (d, J = 9.0 Hz, 1H), 3.69-3.53 (m, 2H), 2.37 (s, 1H), 2.15 (s, 1H), 2.05 (s, 1H), 1.05 (d, J = 6.9 Hz, 3H). |
| 26 | 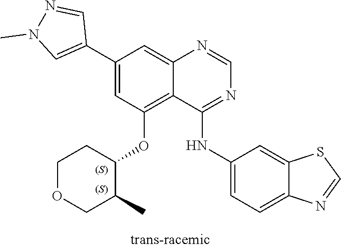 | trans-racemic N-(7-(1-methyl-1H-pyrazol-4-yl)-5-(((3S,4S)-3- methyltetrahydro-2H-pyran-4-yl)oxy)quinazolin-4- yl)benzo[d]thiazol-6-amine LC-MS: (ES, m/z): RT = 1.434 min, LCMS: m/z = 473 [M + 1], ¹H NMR (300 MHz, DMSO-d6) δ 10.35 (s, 1H), 9.31 (s, 1H), 8.93 (d, J = 2.1 Hz, 1H), 8.57 (s, 1H), 8.46 (s, 1H), 8.17 (d, J = 0.8 Hz, 1H), 8.12 (d, J = 8.8 Hz, 1H), 7.74 (dd, J = 8.8, 2.2 Hz, 1H), 7.58 (d, J = 1.4 Hz, 1H), 7.43 (d, J = 1.6 Hz, 1H), 4.83 (td, J = 10.0, 4.3 Hz, 1H), 3.92 (s, 5H), 3.64 (s, 1H), 3.24 (s, 1H), 2.40-2.22 (m, 2H), 1.81 (t, J = 9.7 Hz, 1H), 1.05 (d, J = 6.6 Hz, 3H). |
| 27 | 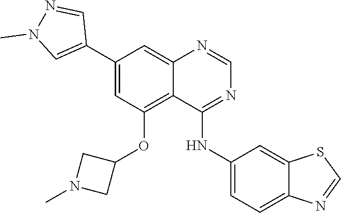 [SMILES: CN1CC(Oc2cc(-c3cnn(C)c3)cc3ncnc(Nc4ccc5ncsc5c4)c23)C1] | N-(7-(1-methyl-1H-pyrazol-4-yl)-5-((1-methylazetidin-3- yl)oxy)quinazolin-4-yl)benzo[d]thiazol-6-amine LC-MS: (ES, m/z): RT = 2.068 min, LCMS: m/z = 444 [M + 1], ¹H NMR (300 MHz, DMSO-d6) δ 10.18 (s, 1H), 9.32 (s, 1H), 8.96 (d, J = 2.2 Hz, 1H), 8.58 (s, 1H), 8.46 (s, 1H), 8.16-8.07 (m, 2H), 7.77 (dd, J = 8.8, 2.2 Hz, 1H), 7.58 (d, J = 1.4 Hz, 1H), 7.04 (d, J = 1.5 Hz, 1H), 5.24 (p, J = 5.3 Hz, 1H), 3.98-3.89 (m, 5H), 3.33-3.25 (m, 2H), 2.39 (s, 3H). |
| 28 | 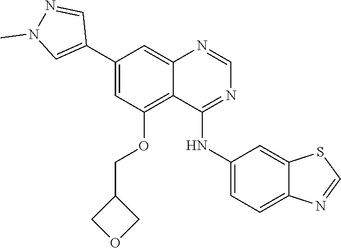 [SMILES: Cn1cc(-c2cc(OCC3COC3)c3c(Nc4ccc5ncsc5c4)ncnc3c2)cn1] | N-(7-(1-methyl-1H-pyrazol-4-yl)-5-(oxetan-3- ylmethoxy)quinazolin-4-yl)benzo[d]thiazol-6-amine LC-MS: (ES, m/z): RT = 2.253 min, LCMS: m/z = 445 [M + 1], ¹H NMR (300 MHz, DMSO-d6) δ 9.32 (s, 1H), 8.76 (d, J = 2.2 Hz, 1H), 8.51 (d, J = 17.7 Hz, 2H), 8.17 (s, 1H), 8.08 (d, J = 8.8 Hz, 1H), 7.75 (dd, J = 8.8, 2.2 Hz, 1H), 7.60 (d, J = 1.4 Hz, 1H), 7.42 (d, J = 1.5 Hz, 1H), 4.92 (dd, J = 7.8, 6.2 Hz, 2H), 4.64 (t, J = 5.8 Hz, 4H), 3.92 (s, 3H), 3.66 (d, J = 14.0 Hz, 1H). |
| 29 | 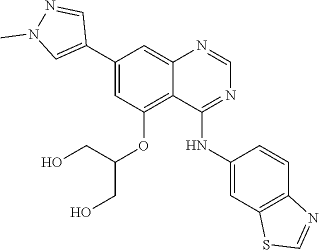 [SMILES: Cn1cc(-c2cc(OC(CO)CO)c3c(Nc4ccc5ncsc5c4)ncnc3c2)cn1] | 2-((4-(benzo[d]thiazol-6-ylamino)-7-(1-methyl-1H-pyrazol-4- yl)quinazolin-5-yl)oxy)propane-1,3-diol LC-MS: (ES, m/z): RT = 0.880 min, LCMS: m/z = 449 [M + 1], ¹H NMR (300 MHz, DMSO-d6) δ 10.69 (s, 1H), 9.30 (s, 1H), 8.98 (t, J = 2.7 Hz, 1H), 8.57 (d, J = 1.1 Hz, 1H), 8.44 (s, 1H), 8.15-8.05 (m, 2H), 7.93 (dd, J = 8.8, 2.2 Hz, 1H), 7.54 (dd, J = 17.6, 1.5 Hz, 2H), 5.28 (t, J = 5.3 Hz, 1H), 4.96 (t, J = 4.6 Hz, 1H), 3.92 (s, 4H), 3.89 (d, J = 4.6 Hz, 4H) |
| 30 | 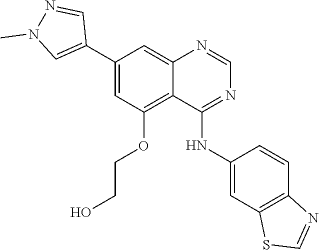 [SMILES: Cn1cc(-c2cc(OCCO)c3c(Nc4ccc5ncsc5c4)ncnc3c2)cn1] | 2-((4-(benzo[d]thiazol-6-ylamino)-7-(1-methyl-1H-pyrazol-4- yl)quinazolin-5-yl)oxy)ethan-1-ol LC-MS: (ES, m/z): RT = 1.987 min, LCMS: m/z = 419 [M + 1], ¹H NMR (400 MHz, DMSO-d6) δ 10.62 (s, 1H), 9.30 (s, 1H), 9.00 (d, J = 2.1 Hz, 1H), 8.59 (s, 1H), 8.46 (s, 1H), 8.15 (d, J = 0.8 Hz, 1H), 8.07 (d, J = 8.8 Hz, 1H), 7.97 (dd, J = 8.8, 2.2 Hz, 1H), 7.58 (d, J = 1.5 Hz, 1H), 7.39 (d, J = 1.6 Hz, 1H), 5.43 (d, J = 4.8 Hz, 1H), 4.50- 4.43 (m, 2H), 4.03 (d, J = 4.5 Hz, 2H), 3.91 (s, 3H). |
| 31 | 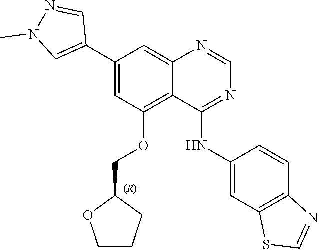 | (R)-N-(7-(1-methyl-1H-pyrazol-4-yl)-5-((tetrahydrofuran-2- yl)methoxy)quinazolin-4-yl)benzo[d]thiazol-6-amine LC-MS: (ES, m/z): RT = 1.475 min, m/z = 459 [M + 1], ¹H NMR (300 MHz, DMSO-d6) δ10.50 (s, 1H), 9.31 (s, 1H), 9.01 (t, J = 2.7 Hz, 1H), 8.58 (s, 1H), 8.44 (s, 1H), 8.17-8.05 (m, 2H), 7.85 (dd, J = 8.8, 2.2 Hz, 1H), 7.58 (d, J = 1.4 Hz, 1H), 7.39 (d, J = 1.6 Hz, 1H), 4.64-4.44 (m, 2H), 4.16 (t, J = 9.3 Hz, 1H), 3.91 (s, 3H), 4.00- 3.81 (m, 2H), 2.13 (dt, J = 12.8, 6.9 Hz, 1H), 1.97 (p, J = 6.9 Hz, 2H), 1.82-1.64 (m, 1H). |
| 32 | 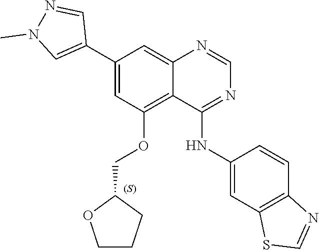 | (S)-N-(7-(1-methyl-1H-pyrazol-4-yl)-5-((tetrahydrofuran-2- yl)methoxy)quinazolin-4-yl)benzo[d]thiazol-6-amine LC-MS: (ES, m/z): RT = 1.492 min, m/z = 459 [M + 1], ¹H NMR (300 MHz, DMSO-d6) δ 10.50 (s, 1H), 9.31 (s, 1H), 9.01 (t, J = 2.6 Hz, 1H), 8.58 (s, 1H), 8.44 (s, 1H), 8.17-8.05 (m, 2H), 7.85 (dd, J = 8.8, 2.2 Hz, 1H), 7.57 (d, J = 1.4 Hz, 1H), 7.38 (d, J = 1.5 Hz, 1H), 4.64-4.33 (m, 2H), 4.16 (t, J = 9.3 Hz, 1H), 3.91 (s, 3H), 4.00- 3.81 (m, 2H), 2.21-2.06 (m, 1H), 1.97 (p, J = 6.9 Hz, 2H), 1.82- 1.64 (m, 1H) |
| 33 | 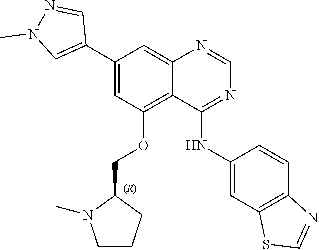 | (R)-N-(7-(1-methyl-1H-pyrazol-4-yl)-5-((1-methylpyrrolidin-2- yl)methoxy)quinazolin-4-yl)benzo[d]thiazol-6-amine LC-MS: (ES, m/z): RT = 1.494 min, LCMS: m/z = 472[M + 1], ¹H NMR (300 MHz, DMSO-d6) δ 9.31 (s, 1H), 8.80 (dd, J = 5.6, 2.1 Hz, 1H), 8.53 (d, J = 2.0 Hz, 1H), 8.45 (s, 1H), 8.12 (d, J = 9.6 Hz, 2H), 7.83 (dt, J = 8.7, 2.6 Hz, 1H), 7.56 (d, J = 1.4 Hz, 1H), 7.41 (d, J = 1.6 Hz, 1H), 4.62-4.52 (m, 1H), 4.37 (dd, J = 10.3, 3.7 Hz, 1H), 3.91 (s, 3H), 3.07 (s, 2H), 2.54 (s, 4H), 2.40 (s, 1H), 2.29 (s, 1H), 1.67 (s, 2H). |
| 34 | 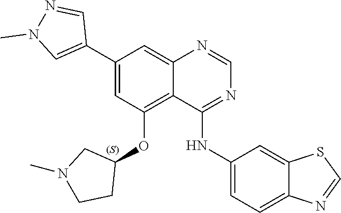 | (S)-N-(7-(1-methyl-1H-pyrazol-4-yl)-5-((1-methylpyrrolidin-3- yl)oxy)quinazolin-4-yl)benzo[d]thiazol-6-amine LC-MS: (ES, m/z): RT = 1.395 min, LCMS: m/z = 458 [M + 1], ¹H NMR (300 MHz, DMSO-d6) δ 10.62 (s, 1H), 9.31 (s, 1H), 9.01- 8.93 (m, 1H), 8.58 (d, J = 1.2 Hz, 1H), 8.46 (s, 1H), 8.18-8.07 (m, 2H), 7.87 (dt, J = 8.8, 1.7 Hz, 1H), 7.58 (d, J = 1.4 Hz, 1H), 7.39 (d, J = 1.5 Hz, 1H), 5.52 (s, 1H), 3.91 (s, 3H), 3.24 (m, 1H), 3.11-3.02 (m, 1H), 2.67-2.58 (m, 2H), 2.43 (s, 4H), 2.06-1.96 (m, 1H). |
| 35 | 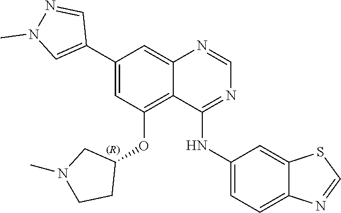 | (R)-N-(7-(1-methyl-1H-pyrazol-4-yl)-5-((1-methylpyrrolidin-3- yl)oxy)quinazolin-4-yl)benzo[d]thiazol-6-amine LC-MS: (ES, m/z): RT = 1.392 min, LCMS: m/z = 458 [M + 1], ¹H NMR (300 MHz, DMSO-d6) δ 10.62 (s, 1H), 9.31 (s, 1H), 9.01- 8.93 (m, 1H), 8.58 (d, J = 1.2 Hz, 1H), 8.46 (s, 1H), 8.18-8.07 (m, 2H), 7.87 (dt, J = 8.8, 1.7 Hz, 1H), 7.58 (d, J = 1.4 Hz, 1H), 7.39 (d, J = 1.5 Hz, 1H), 5.52 (s, 1H), 3.91 (s, 3H), 3.24 (m, 1H), 3.11-3.02 (m, 1H), 2.67-2.58 (m, 2H), 2.43 (s, 4H), 2.06-1.96 (m, 1H) |
| 36 | 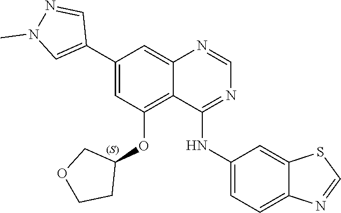 | (S)-N-(7-(1-methyl-1H-pyrazol-4-yl)-5-((tetrahydrofuran-3- yl)oxy)quinazolin-4-yl)benzo[d]thiazol-6-amine LC-MS: (ES, m/z): RT = 1.328 min, LCMS: m/z = 445 [M + 1],¹H NMR (300 MHz, DMSO-d6) 1H δ 10.26 (s, 1H), 9.30 (s, 1H), 8.98 (s, 1H), 8.57 (s, 1H), 8.46 (s, 1H), 8.15 (s, 1H), 8.09 (d, J = 8.8 Hz, 1H), 7.73 (d, J = 8.7 Hz, 1H), 7.58 (s, 1H), 7.40 (s, 1H), 5.68-5.60 (m, 1H), 4.25 (d, J = 10.6 Hz, 1H), 3.98 (t, J = 11.0 Hz, 2H), 3.89- 3.81 (m, 4H), 2.28-2.16 (m, 2H). |
| 37 | 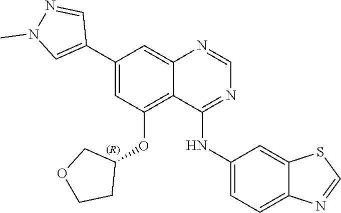 | (R)-N-(7-(1-methyl-1H-pyrazol-4-yl)-5-((tetrahydrofuran-3- yl)oxy)quinazolin-4-yl)benzo[d]thiazol-6-amine LC-MS: (ES, m/z): RT = 1.332 min, LCMS: m/z = 445 [M + 1], ¹H NMR (300 MHz, DMSO-d6) 1H δ 10.30 (s, 1H), 9.30 (s, 1H), 8.96 (d, J = 2.1 Hz, 1H), 8.58 (s, 1H), 8.47 (s, 1H), 8.15 (s, 1H), 8.09 (d, J = 8.8 Hz, 1H), 7.73 (dd, J = 8.8, 2.2 Hz, 1H), 7.57 (d, J = 1.4 Hz, 1H), 7.41 (d, J = 1.5 Hz, 1H), 5.68-5.62 (m, 1H), 4.25 (d, J = 10.6 Hz, 1H), 4.03-3.93 (m, 2H), 4.02-3.78 (m, 4H), 2.40 (dd, J = 14.0, 8.5 Hz, 1H), 2.23 (d, J = 13.9 Hz, 1H). |
| 38 | 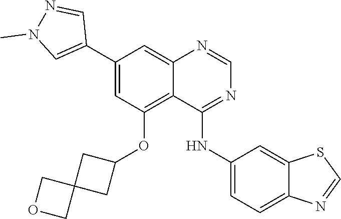 [SMILES: Cn1cc(-c2cc(OC3CC4(COC4)C3)c3c(Nc4ccc5ncsc5c4)ncnc3c2)cn1] | N-(5-((2-oxaspiro[3.3]heptan-6-yl)oxy)-7-(1-methyl-1H-pyrazol-4- yl)quinazolin-4-yl)benzo[d]thiazol-6-amine LC-MS: (ES, m/z): RT = 2.334 min, LCMS: m/z = 471 [M + 1], ¹H NMR (300 MHz, DMSO-d6) δ 10.17 (s, 1H), 9.32 (s, 1H), 8.94 (d, J = 2.1 Hz, 1H), 8.57 (s, 1H), 8.45 (s, 1H), 8.18-8.08 (m, 2H), 7.79 (dd, J = 8.8, 2.2 Hz, 1H), 7.57 (d, J = 1.4 Hz, 1H), 7.08 (s, 1H), 5.09- 4.98 (m, 1H), 4.75 (s, 2H), 4.63 (s, 2H), 3.92 (s, 3H),3.02 (d, J = 8.6 Hz, 2H), 2.61 (d, J = 9.4 Hz, 2H) |
| 41 42 | 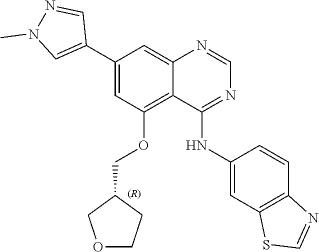 or 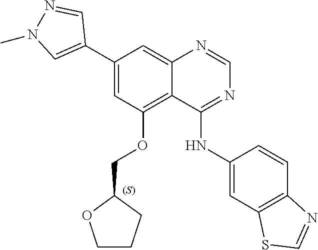 | (S)-N-(7-(1-methyl-1H-pyrazol-4-yl)-5-((tetrahydrofuran-3- yl)methoxy)quinazolin-4-yl)benzo[d]thiazol-6-amine OR (R)-N-(7- (1-methyl-1H-pyrazol-4-yl)-5-((tetrahydrofuran-3- yl)methoxy)quinazolin-4-yl)benzo[d]thiazol-6-amine First Eluting Isomer: LC-MS: (ES, m/z): RT = 1.324 min, LCMS: m/z = 459 [M + 1], ¹H NMR (300 MHz, DMSO-d6) δ 10.08 (s, 1H), 9.32 (s, 1H), 8.83 (dd, J = 4.6, 2.1 Hz, 1H), 8.54 (d, J = 1.8 Hz, 1H), 8.47 (s, 1H), 8.16 (s, 1H), 8.11 (d, J = 8.8 Hz, 1H), 7.73 (dd, J = 9.1, 2.2 Hz, 1H), 7.58 (d, J = 1.4 Hz, 1H), 7.38 (d, J = 1.6 Hz, 1H), 4.38 (d, J = 6.7 Hz, 2H), 3.91 (s, 3H), 3.82-3.52 (m, 4H), 3.11-2.96 (m, 1H), 2.20-2.08 (m, 1H), 1.86 (dd, J = 12.5, 5.4 Hz, 1H). Second Eluting Isomer: LC-MS: (ES, m/z): RT = 1.327 min, LCMS: m/z = 459 [M + 1], ¹H NMR (300 MHz, DMSO-d6) δ 10.06 (s, 1H), 9.32 (s, 1H), 8.82 (dd, J = 4.6, 2.1 Hz, 1H), 8.53 (d, J = 1.7 Hz, 1H), 8.45 (s, 1H), 8.15 (s, 1H), 8.10 (d, J = 8.8 Hz, 1H), 7.71 (dd, J = 8.8, 2.2 Hz, 1H), 7.57 (d, J = 1.4 Hz, 1H), 7.36 (d, J = 1.6 Hz, 1H), 4.36 (d, J = 6.7 Hz, 2H), 3.91 (s, 3H), 3.84 (dt, J = 8.1, 3.7 Hz, 2H), 3.71 (d, J = 7.5 Hz, 1H), 3.67 (s, 1H), 3.11-2.96 (m, 1H), 2.20-2.08 (m, 1H), 1.92-1.80 (m, 1H). |
| 43 44 | 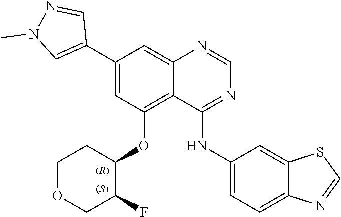 or 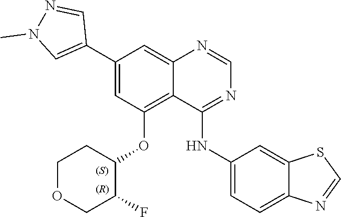 | N-(5-(((3S,4R)-3-fluorotetrahydro-2H-pyran-4-yl)oxy)-7-(1-methyl- 1H-pyrazol-4-yl)quinazolin-4-yl)benzo[d]thiazol-6-amine OR N-(5- (((3R,4S)-3-fluorotetrahydro-2H-pyran-4-yl)oxy)-7-(1-methyl-1H- pyrazol-4-yl)quinazolin-4-yl)benzo[d]thiazol-6-amine First Eluting Isomer: LC-MS: (ES, m/z): RT = 1.320 min, LCMS: m/z = 477 [M + 1], chiral-HPLC RT = 4.757, ¹H NMR (300 MHz, DMSO-d6) δ 10.37 (s, 1H), 9.31 (s, 1H), 8.97 (t, J = 2.5 Hz, 1H), 8.60 (s, 1H), 8.44 (s, 1H), 8.20-7.99 (m, 2H), 7.73 (dd, J = 8.8, 2.2 Hz, 1H), 7.57 (dd, J = 25.5, 1.5 Hz, 2H), 5.47-4.85 (m, 2H), 4.21 (t, J = 12.8 Hz, 1H), 4.02 (dd, J = 11.8, 4.3 Hz, 1H), 3.92 (s, 3H), 3.82- 3.58 (m, 2H), 2.24-1.80 (m, 2H). Second Eluting Isomer: LC-MS: (ES, m/z): RT = 1.316 min, LCMS: m/z = 477 [M + 1], chiral-HPLC RT = 5.810, ¹H NMR (300 MHz, DMSO-d6) δ 10.35 (s, 1H), 9.31 (s, 1H), 8.97 (t, J = 2.5 Hz, 1H), 8.59 (s, 1H), 8.44 (s, 1H), 8.20-7.98 (m, 2H), 7.73 (dd, J = 8.8, 2.2 Hz, 1H), 7.61 (d, J = 1.4 Hz, 1H), 7.57 (s, 1H), 7.52 (d, J = 1.6 Hz, 1H), 5.47-5.10 (m, 2H), 4.21 (t, J = 12.9 Hz, 1H), 4.02 (dd, J = 11.6, 4.3 Hz, 1H), 3.92 (s, 3H), 3.82-3.58 (m, 2H), 2.24-1.99 (m, 2H). |
| 45 46 | 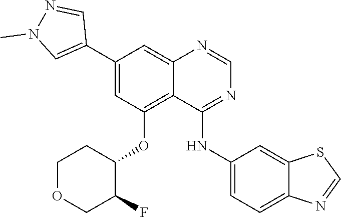 [SMILES: Cn1cc(-c2cc(O[C@H]3CCOC[C@@H]3F)c3c(Nc4ccc5ncsc5c4)ncnc3c2)cn1] or 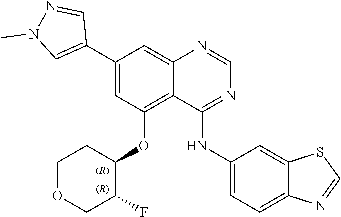 | N-(5-(((3S,4S)-3-fluorotetrahydro-2H-pyran-4-yl)oxy)-7-(1-methyl- 1H-pyrazol-4-yl)quinazolin-4-yl)benzo[d]thiazol-6-amine OR N- (5-(((3R,4R) 3-fluorotetrahydro-2H-pyran-4-yl)oxy)-7-(1-methyl- 1H-pyrazol-4-yl)quinazolin-4-yl)benzo[d]thiazol-6-amine First Eluting Isomer: LC-MS: (ES, m/z): RT = 1.297 min, LCMS: m/z = 477 [M + 1], chiral-HPLC RT = 3.017 min, 1H NMR (300 MHz, DMSO-d6) δ 10.11 (s, 0H), 9.33 (s, 1H), 8.87 (dd, J = 3.8, 2.1 Hz, 1H), 8.57 (d, J = 1.5 Hz, 1H), 8.46 (s, 1H), 8.20-8.07 (m, 2H), 7.86 (dt, J = 8.9, 1.7 Hz, 1H), 7.61 (d, J = 1.4 Hz, 1H), 7.50 (d, J = 1.6 Hz, 1H), 5.39-4.97 (m, 2H), 4.19 (ddd, J = 11.4, 6.9, 4.8 Hz, 1H), 3.93 (s, 4H), 3.73-3.49 (m, 2H), 2.55-2.51 (m, 1H), 2.01- 1.95 (m, 1H). Second Eluting Isomer: LC-MS: (ES, m/z): RT = 1.292 min, LCMS: m/z = 477 [M + 1], chiral-HPLC RT = 3.984 min, ¹H NMR (300 MHz, DMSO-d6) δ 10.11 (s, 1H), 9.33 (s, 1H), 8.87 (dd, J = 3.8, 2.1 Hz, 1H), 8.57 (d, J = 1.5 Hz, 1H), 8.46 (s, 1H), 8.20-8.07 (m, 2H), 7.87 (dt, J = 8.9, 1.8 Hz, 1H), 7.62 (d, J = 1.4 Hz, 1H), 7.50 (d, J = 1.6 Hz, 1H), 5.59-4.85 (m, 2H), 4.19 (dt, J = 11.7, 5.5 Hz, 1H), 3.93 (s, 4H), 3.73-3.49 (m, 2H), 2.55-2.50 (s, 1H), 2.04- 1.99 (m, 1H). |
| 47 48 | 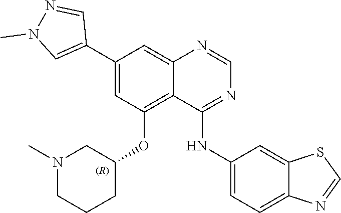 or 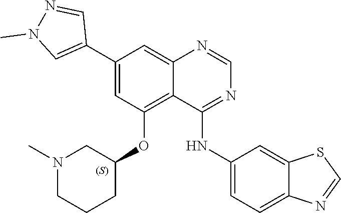 | (S)-N-(7-(1-methyl-1H-pyrazol-4-yl)-5-((1-methylpiperidin-3- yl)oxy)quinazolin-4-yl)benzo[d]thiazol-6-amine OR (R)-N-(7-(1- methyl-1H-pyrazol-4-yl)-5-((1-methylpiperidin-3-yl)oxy)quinazolin- 4-yl)benzo[d]thiazol-6-amine First Eluting Isomer: LC-MS: (ES, m/z): RT = 0.958 min, LCMS: m/z = 472 [M + 1], Chiral-HPLC (ES): RT = 1.78, ¹H NMR (400 MHz, DMSO-d6) δ 10.90 (s, 1H), 9.31 (s, 1H), 8.93 (d, J = 2.1 Hz, 1H), 8.56 (s, 1H), 8.46 (s, 1H), 8.17-8.08 (m, 2H), 8.00 (dd, J = 8.9, 2.1 Hz, 1H), 7.55 (d, J = 1.5 Hz, 1H), 7.42 (d, J = 1.6 Hz, 1H), 5.29 (s, 1H), 3.91 (s, 3H), 3.19 (d, J = 12.1 Hz, 1H), 2.87 (d, J = 11.0 Hz, 1H), 2.41 (d, J = 12.1 Hz, 1H), 2.33 (s, 3H), 2.09 (d, J = 12.3 Hz, 2H), 1.80-1.69 (m, 1H), 1.57 (q, J = 13.7 Hz, 2H) Second Eluting Isomer: LC-MS: (ES, m/z): RT = 1.064 min, LCMS: m/z = 476 [M + 1], Chiral-HPLC (ES): RT = 2.545, ¹H NMR (400 MHz, DMSO-d6) δ 10.90 (s, 1H), 9.31 (s, 1H), 8.93 (d, J = 2.1 Hz, 1H), 8.56 (s, 1H), 8.46 (s, 1H), 8.17-8.08 (m, 2H), 8.00 (dd, J = 8.9, 2.1 Hz, 1H), 7.55 (d, J = 1.5 Hz, 1H), 7.42 (d, J = 1.6 Hz, 1H), 5.29 (s, 1H), 3.91 (s, 3H), 3.19 (d, J = 12.1 Hz, 1H), 2.87 (d, J = 11.0 Hz, 1H), 2.41 (d, J = 12.1 Hz, 1H), 2.33 (s, 3H), 2.09 (d, J = 12.3 Hz, 2H), 1.80-1.69 (m, 1H), 1.57 (q, J = 13.7 Hz, 2H). |
| 49 | 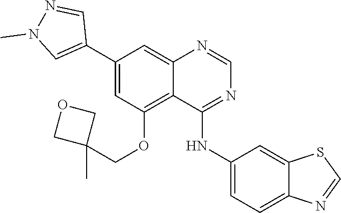 [SMILES: Cn1cc(-c2cc(OCC3(C)COC3)c3c(Nc4ccc5ncsc5c4)ncnc3c2)cn1] | N-(7-(1-methyl-1H-pyrazol-4-yl)-5-((3-methyloxetan-3- yl)methoxy)quinazolin-4-yl)benzo[d]thiazol-6-amine LC-MS: (ES, m/z): RT = 2.619 min. LC-MS: m/z = 459 [M + 1]; ¹H NMR (500 MHz, DMSO-d₆) δ 10.09 (s, 1H), 9.34 (s, 1H), 8.62 (s, 1H), 8.51 (d, J = 6.7 Hz, 2H), 8.19 (s, 1H), 8.07 (d, J = 8.7 Hz, 1H), 7.71 (d, J = 8.7 Hz, 1H), 7.62 (s, 1H), 7.44 (s, 1H), 4.84-4.68 (m, 2H), 4.58 (d, J = 6.0 Hz, 2H), 4.43 (s, 2H), 3.94 (s, 3H), 1.43 (s, 3H). |
| 50 | 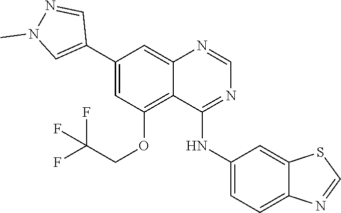 [SMILES: Cn1cc(-c2cc(OCC(F)(F)F)c3c(Nc4ccc5ncsc5c4)ncnc3c2)cn1] | N-(7-(1-methyl-1H-pyrazol-4-yl)-5-(2,2,2- trifluoroethoxy)quinazolin-4-yl)benzo[d]thiazol-6-amine LC-MS: (ES, m/z): RT = 2.907, LC-MS: m/z = 457 [M + 1]; ¹H NMR (500 MHz, DMSO-d6) δ 9.81 (s, 1H), 9.35 (s, 1H), 8.97 (s, 1H), 8.64 (s, 1H), 8.49 (s, 1H), 8.20 (s, 1H), 8.16 (d, J = 8.4 Hz, 1H), 7.71 (s, 1H), 7.63 (d, J = 8.6 Hz, 1H), 7.56 (s, 1H), 5.33 (d, J = 8.9 Hz, 2H), 3.94 (s, 3H). |
| 51 | 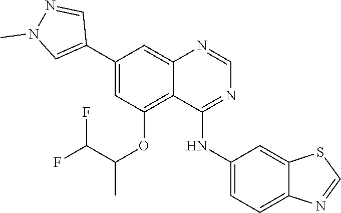 [SMILES: CC(Oc1cc(-c2cnn(C)c2)cc2ncnc(Nc3ccc4ncsc4c3)c12)C(F)F] | (rac)-N-(5-((1,1-difluoropropan-2-yl)oxy)-7-(1-methyl-1H-pyrazol- 4-yl)quinazolin-4-yl)benzo[d]thiazol-6-amine LC-MS: (ES, m/z): RT = 2.752, LC-MS: m/z = 453 [M + 1]; ¹H NMR (500 MHz, DMSO-d6) δ 10.10 (s, 1H), 9.34 (d, J = 6.3 Hz, 1H), 8.96 (s, 1H), 8.61 (d, J = 3.4 Hz, 1H), 8.51 (s, 1H), 8.20 (s, 1H), 8.14 (d, J = 8.4 Hz, 1H), 7.73 (d, J = 8.7 Hz, 1H), 7.66 (s, 1H), 7.55 (s, 1H), 6.67 (t, J = 54.4 Hz, 1H), 5.51 (s, 1H), 3.94 (d, J = 3.3 Hz, 3H), 1.59 (d, J = 6.4 Hz, 3H). |
| 52 53 | 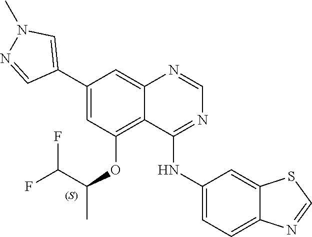 or 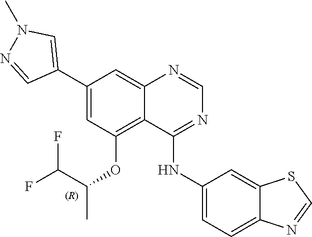 | (R)-N-(5-((1,1-difluoropropan-2-yl)oxy)-7-(1-methyl-1H-pyrazol-4- yl)quinazolin-4-yl)benzo[d]thiazol-6-amine OR (S)-N-(5-((1,1- difluoropropan-2-yl)oxy)-7-(1-methyl-1H-pyrazol-4-yl)quinazolin- 4-yl)benzo[d]thiazol-6-amine First Eluting isomer: LC-MS: (ES, m/z): RT = 0.808 min, LCMS: m/z = 453.4 [M + 1], Chiral-HPLC (ES): RT = 4.45 min, 1H NMR (400 MHz, DMSO-d6) δ 10.08 (s, 1H), 9.33 (s, 1H), 8.95 (d, J = 2.1 Hz, 1H), 8.60 (s, 1H), 8.50 (s, 1H), 8.19 (d, J = 0.8 Hz, 1H), 8.12 (d, J = 8.8 Hz, 1H), 7.72 (dd, J = 8.9, 2.2 Hz, 1H), 7.64 (d, J = 1.4 Hz, 1H), 7.53 (d, J = 1.6 Hz, 1H), 6.65 (d, J = 2.1 Hz, 0H), 5.56-5.44 (m, 1H), 3.92 (s, 3H), 1.57 (d, J = 6.3 Hz, 3H). Second Eluting isomer: LC-MS: (ES, m/z): RT = 0.825 min, LCMS: m/z = 453.4 [M + 1], Chiral-HPLC (ES): RT = 5.29 min, 1H NMR (300 MHz, DMSO-d6) δ 10.07 (s, 1H), 9.32 (s, 1H), 8.93 (s, 1H), 8.57 (d, J = 10.7 Hz, 2H), 8.18 (s, 2H), 7.71 (d, J = 8.7 Hz, 1H), 7.63 (s, 2H), 6.64 (s, 1H), 5.48 (s, 1H), 3.91 (s, 3H), 1.56 (d, J = 6.3 Hz, 3H). |
| 54 | 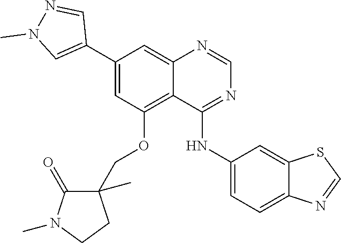 [SMILES: CN1CCC(C)(COc2cc(-c3cnn(C)c3)cc3ncnc(Nc4ccc5ncsc5c4)c23)C1=O] | 3-(((4-(benzo[d]thiazol-6-ylamino)-7-(1-methyl-1H-pyrazol-4- yl)quinazolin-5-yl)oxy)methyl)-1,3-dimethylpyrrolidin-2-one LC-MS: (ES, m/z): RT = 2.584 min, LC-MS: m/z = 500 [M + 1]; ¹H NMR (500 MHz, DMSO-d6) δ 10.17 (s, 1H), 9.33 (s, 1H), 8.74 (d, J = 2.1 Hz, 1H), 8.47 (d, J = 13.9 Hz, 2H), 8.15 (s, 1H), 8.10 (d, J = 8.8 Hz, 1H), 7.92 (dd, J = 8.8, 2.1 Hz, 1H), 7.56 (d, J = 1.4 Hz, 1H), 7.34 (d, J = 1.6 Hz, 1H), 4.38 (d, J = 9.6 Hz, 1H), 4.30 (d, J = 9.6 Hz, 1H), 3.92 (s, 3H), 3.40 (ddt, J = 9.5, 5.7, 3.5 Hz, 2H), 2.71 (s, 3H), 2.25-2.16 (m, 1H), 1.97 (dt, J = 13.6, 7.5 Hz, 1H), 1.33 (s, 3H). |
| 55 56 | 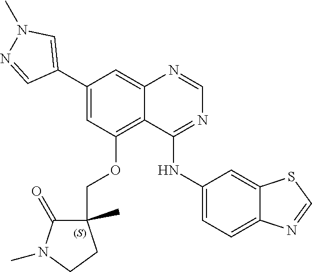 or 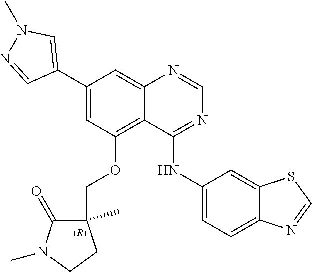 | (R)-3-(((4-(benzo[d]thiazol-6-ylamino)-7-(1-methyl-1H-pyrazol-4- yl)quinazolin-5-yl)oxy)methyl)-1,3-dimethylpyrrolidin-2-one, OR , (S)-3-(((4-(benzo[d]thiazol-6-ylamino)-7-(1-methyl-1H-pyrazol-4- yl)quinazolin-5-yl)oxy)methyl)-1,3-dimethylpyrrolidin-2-one First Eluting isomer: LC-MS: (ES, m/z): RT = 0.964 min, LCMS: m/z = 500.35 [M + 1], Chiral-HPLC (ES): RT = 1.812 min, 1H NMR (300 MHz, DMSO-d6) δ 10.17 (s, 1H), 9.34 (s, 1H), 8.75 (d, J = 2.0 Hz, 1H), 8.48 (d, J = 5.9 Hz, 2H), 8.16 (s, 2H), 7.92 (dd, J = 8.8, 2.1 Hz, 1H), 7.57 (s, 1H), 7.35 (s, 1H), 4.39 (d, J = 9.7 Hz, 2H), 3.92 (s, 3H), 3.40 (s, 2H), 2.71 (s, 3H), 2.23(s, 1H), 1.97(s,1H), 1.33 (s, 3H). First Eluting isomer: LC-MS: (ES, m/z): RT = 0.957 min, LCMS: m/z = 500.35 [M + 1], Chiral-HPLC (ES): RT = 2.235 min, 1H NMR (300 MHz, DMSO-d6) 8 10.17 (s, 1H), 9.34 (s, 1H), 8.75 (d, J = 2.1 Hz, 1H), 8.48 (d, J = 6.0 Hz, 2H), 8.16 (s, 1H), 8.10 (d, J = 8.8 Hz, 1H), 7.92 (dd, J = 8.9, 2.1 Hz, 1H), 7.57 (d, J = 1.4 Hz, 1H), 7.35 (d, J = 1.6 Hz, 1H), 4.39 (d, J = 9.6 Hz, 2H), 3.92 (s, 3H), 3.40 (t, J = 7.1 Hz, 2H), 2.71 (s, 3H), 2.19 (q, J = 6.9 Hz, 1H), 1.97 (dt, J = 13.3, 7.1 Hz, 1H), 1.33 (s, 3H). |
| 57 | 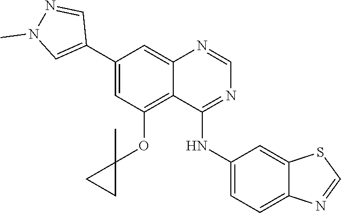 [SMILES: Cn1cc(-c2cc(OC3(C)CC3)c3c(Nc4ccc5ncsc5c4)ncnc3c2)cn1] | N-(7-(1-methyl-1H-pyrazol-4-yl)-5-(1- methylcyclopropoxy)quinazolin-4-yl)benzo[d]thiazol-6-amine LC-MS: (ES, m/z): RT = 2.469 min, LC-MS: m/z = 429 [M + 1]; ¹H NMR (500 MHz, DMSO-d6) δ 10.10 (s, 1H), 9.34 (s, 1H), 8.89 (d, J = 2.2 Hz, 1H), 8.58 (s, 1H), 8.47 (s, 1H), 8.13 (d, J = 8.3 Hz, 2H), 7.79 (dd, J = 8.8, 2.2 Hz, 1H), 7.60 (d, J = 1.5 Hz, 1H), 7.49 (d, J = 1.5 Hz, 1H), 3.95 (s, 3H), 1.76 (s, 3H), 1.35-1.29 (m, 2H), 1.13- 1.02 (m, 2H). |
| 58 | 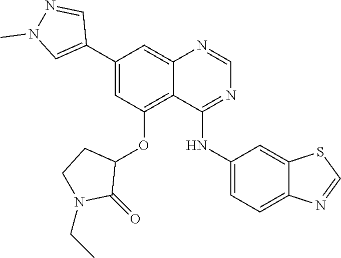 [SMILES: CCN1CCC(Oc2cc(-c3cnn(C)c3)cc3ncnc(Nc4ccc5ncsc5c4)c23)C1=O] | (rac)-3-((4-(benzo[d]thiazol-6-ylamino)-7-(1-methyl-1H-pyrazol-4- yl)quinazolin-5-yl)oxy)-1-ethylpyrrolidin-2-one LC-MS: (ES, m/z): RT = 2.626 min, LC-MS: m/z = 486 [M + 1]; ¹H NMR (500 MHz, DMSO-d6) δ 10.56 (s, 1H), 9.31 (s, 1H), 9.04 (d, J = 2.1 Hz, 1H), 8.60 (s, 1H), 8.48 (s, 1H), 8.16 (s, 1H), 8.10 (d, J = 8.8 Hz, 1H), 7.94 (dd, J = 8.9, 2.2 Hz, 1H), 7.63 (s, 1H), 7.33 (s, 1H), 5.42 (t, J = 8.4 Hz, 1H), 3.93 (s, 3H), 3.61-3.36 (m, 4H), 3.13 (dt, J = 13.5, 7.1 Hz, 1H), 2.28-2.11 (m, 1H), 1.17 (t, J = 7.3 Hz, 3H). |
| 59 60 | 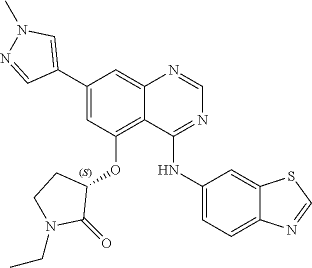 or 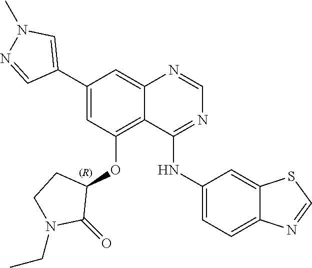 | (S)-3-((4-(benzo[d]thiazol-6-ylamino)-7-(1-methyl-1H-pyrazol-4- yl)quinazolin-5-yl)oxy)-1-ethylpyrrolidin-2-one or (R)-3-((4- (benzo[d]thiazol-6-ylamino)-7-(1-methyl-1H-pyrazol-4- yl)quinazolin-5-yl)oxy)-1-ethylpyrrolidin-2-one First Eluting Isomer: LC-MS: (ES, m/z): RT = 1.623 min, LCMS: m/z = 486.35 [M + 1], Chiral-HPLC (ES): RT = 1.726 min, 1H NMR (300 MHz, DMSO-d6) δ 10.54 (s, 1H), 9.31 (s, 1H), 9.04 (d, J = 2.1 Hz, 1H), 8.59 (s, 1H), 8.46 (s, 1H), 8.16 (d, J = 0.8 Hz, 1H), 8.09 (d, J = 8.9 Hz, 1H), 7.93 (dd, J = 8.9, 2.1 Hz, 1H), 7.62 (d, J = 1.4 Hz, 1H), 7.31 (d, J = 1.5 Hz, 1H), 5.41 (t, J = 8.4 Hz, 1H), 3.92 (s, 3H), 3.48 (dtt, J = 28.1, 13.8, 7.6 Hz, 4H), 3.22-3.08 (m, 1H), 2.26- 2.05 (m, 1H), 1.16 (t, J = 7.2 Hz, 3H). Second Eluting Isomer: LC-MS: (ES, m/z): RT = 1.627 min, LCMS: m/z = 486.35 [M + 1], Chiral-HPLC (ES): RT = 2.167 min, 1H NMR (300 MHz, DMSO-d6) δ 10.55 (s, 1H), 9.31 (s, 1H), 9.05 (d, J = 2.1 Hz, 1H), 8.59 (s, 1H), 8.47 (s, 1H), 8.16 (s, 1H), 8.09 (d, J = 8.8 Hz, 1H), 7.93 (dd, J = 8.9, 2.2 Hz, 1H), 7.62 (d, J = 1.4 Hz, 1H), 7.32 (d, J = 1.6 Hz, 1H), 5.41 (t, J = 8.4 Hz, 1H), 3.92 (s, 3H), 3.47 (ddt, J = 28.2, 13.9, 7.1 Hz, 4H), 3.21-3.06 (m, 1H), 2.26-2.07 (m, 1H), 1.16 (t, J = 7.2 Hz, 3H). |
| 61 | 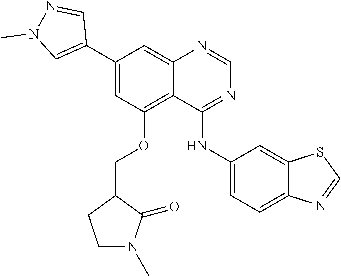 [SMILES: CN1CCC(COc2cc(-c3cnn(C)c3)cc3ncnc(Nc4ccc5ncsc5c4)c23)C1=O] | 3-(((4-(benzo[d]thiazol-6-ylamino)-7-(1-methyl-1H-pyrazol-4- yl)quinazolin-5-yl)oxy)methyl)-1-methylpyrrolidin-2-one LC-MS: (ES, m/z): RT = 2.518 min, LC-MS: m/z = 486 [M + 1]; ¹H NMR (500 MHz, DMSO-d6) δ 10.24 (s, 1H), 9.34 (d, J = 1.2 Hz, 1H), 8.78 (d, J = 2.1 Hz, 1H), 8.50 (d, J = 21.2 Hz, 2H), 8.17 (s, 1H), 8.10 (d, J = 8.8 Hz, 1H), 7.85 (dd, J = 8.8, 2.2 Hz, 1H), 7.59 (s, 1H), 7.39 (s, 1H), 4.59 (dd, J = 9.8, 5.3 Hz, 1H), 4.55-4.44 (m, 1H), 3.93 (s, 3H), 3.41 (dd, J = 8.0, 6.2 Hz, 2H), 3.19 (p, J = 7.8 Hz, 1H), 2.75 (s, 3H), 2.41-2.27 (m, 1H), 2.00 (dd, J = 13.0, 8.0 Hz, 1H). |
| 62 | 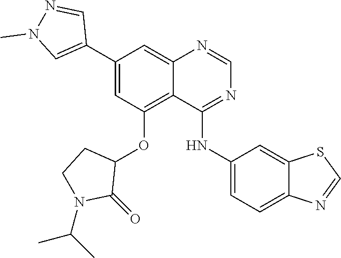 [SMILES: CC(C)N1CCC(Oc2cc(-c3cnn(C)c3)cc3ncnc(Nc4ccc5ncsc5c4)c23)C1=O] | (rac)-3-((4-(benzo[d]thiazol-6-ylamino)-7-(1-methyl-1H-pyrazol-4- yl)quinazolin-5-yl)oxy)-1-isopropylpyrrolidin-2-one LC-MS: (ES, m/z): RT = 2.337 min, LC-MS: m/z = 500 [M + 1]; ¹H NMR (500 MHz, DMSO-d6) δ 10.59 (s, 1H), 9.33 (s, 1H), 9.05 (d, J = 2.0 Hz, 1H), 8.62 (s, 1H), 8.49 (s, 1H), 8.18 (s, 1H), 8.11 (d, J = 8.8 Hz, 1H), 7.96 (dd, J = 8.8, 2.1 Hz, 1H), 7.65 (s, 1H), 7.35 (s, 1H), 5.43 (t, J = 8.5 Hz, 1H), 4.34 (p, J = 6.8 Hz, 1H), 3.94 (s, 3H), 3.66-3.50 (m, 1H), 3.42 (td, J = 9.3, 6.7 Hz, 1H), 3.14 (dt, J = 13.5, 7.3 Hz, 1H), 2.33-2.04 (m, 1H), 1.23 (dd, J = 8.3, 6.8 Hz, 6H). |
| 63 64 | 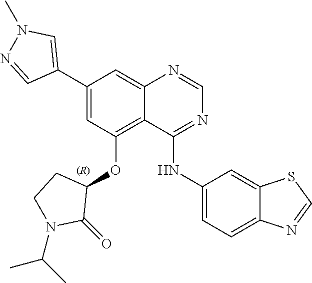 or 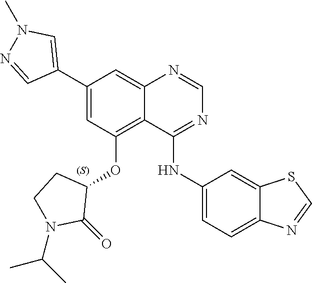 | (R)-3-((4-(benzo[d]thiazol-6-ylamino)-7-(1-methyl-1H-pyrazol-4- yl)quinazolin-5-yl)oxy)-1-isopropylpyrrolidin-2-one OR (S)-3-((4- (benzo[d]thiazol-6-ylamino)-7-(1-methyl-1H-pyrazol-4- yl)quinazolin-5-yl)oxy)-1-isopropylpyrrolidin-2-one First Eluting Isomer: LC-MS: (ES, m/z): RT = 1.034 min, LCMS: m/z = 500.10 [M + 1], Chiral-HPLC (ES): RT = 1.717, ¹H NMR (400 MHz, DMSO-d6) δ 10.58 (s, 1H), 9.31 (s, 1H), 9.04 (d, J = 2.1 Hz, 1H), 8.60 (s, 1H), 8.48 (s, 1H), 8.16 (d, J = 0.9 Hz, 1H), 8.09 (d, J = 8.8 Hz, 1H), 7.94 (dd, J = 8.9, 2.2 Hz, 1H), 7.63 (d, J = 1.5 Hz, 1H), 7.35-7.29 (m, 1H), 5.42 (t, J = 8.5 Hz, 1H), 4.33 (p, J = 6.7 Hz, 1H), 3.93 (s, 3H), 3.56 (s, 1H), 3.32 (s,1H), 3.18 (s,1H), 2.21-2.08 (m, 1H), 1.21 (t, J = 6.7 Hz, 6H). Second Eluting Isomer: LC-MS: (ES, m/z): RT = 1.027 min, LCMS: m/z = 500.10 [M + 1], Chiral-HPLC (ES): RT = 2.417, ¹H NMR (400 MHz, DMSO-d6) δ 10.58 (s, 1H), 9.31 (s, 1H), 9.04 (d, J = 2.1 Hz, 1H), 8.60 (s, 1H), 8.47 (s, 1H), 8.16 (d, J = 0.8 Hz, 1H), 8.09 (d, J = 8.9 Hz, 1H), 7.94 (dd, J = 8.9, 2.1 Hz, 1H), 7.63 (d, J = 1.5 Hz, 1H), 7.33 (d, J = 1.6 Hz, 1H), 5.42 (t, J = 8.5 Hz, 1H), 4.33 (p, J = 6.7 Hz, 1H), 3.93 (s, 3H), 3.56 (s, 1H), 3.31 (s,2H), 2.20-2.08 (m, 1H), 1.21 (t, J = 6.7 Hz, 6H). |
| 65 | 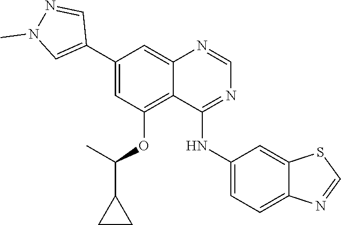 [SMILES: C[C@@H](Oc1cc(-c2cnn(C)c2)cc2ncnc(Nc3ccc4ncsc4c3)c12)C1CC1] | (R)-N-(5-(1-cyclopropylethoxy)-7-(1-methyl-1H-pyrazol-4- yl)quinazolin-4-yl)benzo[d]thiazol-6-amine LC-MS: (ES, m/z): RT = 2.885 min, LC-MS: m/z = 443 [M + 1]; ¹H NMR (500 MHz, DMSO-d6) δ 10.69 (s, 1H), 9.33 (d, J = 1.9 Hz, 1H), 8.99 (d, J = 2.3 Hz, 1H), 8.61 (s, 1H), 8.47 (s, 1H), 8.21-8.05 (m, 2H), 7.81 (dt, J = 8.9, 2.1 Hz, 1H), 7.57 (s, 1H), 7.37 (s, 1H), 4.57 (t, J = 7.1 Hz, 1H), 3.93 (d, J = 1.7 Hz, 3H), 1.63-1.50 (m, 4H), 0.74-0.63 (m, 2H), 0.53 (q, J = 4.2 Hz, 2H). |
| 66 | 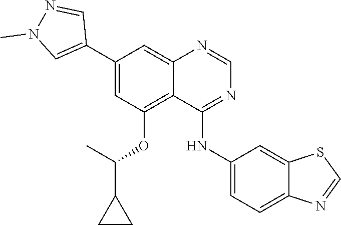 [SMILES: C[C@H](Oc1cc(-c2cnn(C)c2)cc2ncnc(Nc3ccc4ncsc4c3)c12)C1CC1] | (S)-N-(5-(1-cyclopropylethoxy)-7-(1-methyl-1H-pyrazol-4- yl)quinazolin-4-yl)benzo[d]thiazol-6-amine LC-MS: (ES, m/z): RT = 2.884 min, LC-MS: m/z = 443 [M + 1]; ¹H NMR (500 MHz, DMSO-d6) 10.69 (s, 1H), 9.33 (s, 1H), 8.99 (s, 1H), 8.61 (s, 1H), 8.47 (s, 1H), 8.23-8.04 (m, 2H), 7.81 (d, J = 8.8 Hz, 1H), 7.57 (s, 1H), 7.37 (s, 1H), 4.57 (t, J = 7.1 Hz, 1H), 3.93 (s, 3H), 1.59 (d, J = 6.0 Hz, 4H), 0.71 (dd, J = 7.9, 4.3 Hz, 2H), 0.54 (d, J = 5.7 Hz, 2H). |
| 67 | 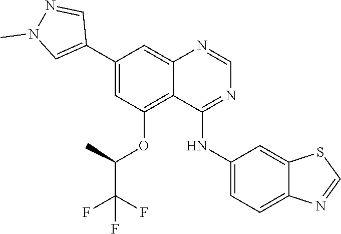 [SMILES: C[C@@H](Oc1cc(-c2cnn(C)c2)cc2ncnc(Nc3ccc4ncsc4c3)c12)C(F)(F)F] | (R)-N-(7-(1-methyl-1H-pyrazol-4-yl)-5-((1,1,1-trifluoropropan-2- yl)oxy)quinazolin-4-yl)benzo[d]thiazol-6-amine LC-MS: (ES, m/z): RT = 2.501 min, LC-MS: m/z = 471 [M + 1]; ¹H NMR (500 MHz, DMSO-d6) δ 9.85 (s, 1H), 9.34 (d, J = 1.4 Hz, 1H), 8.92 (s, 1H), 8.62 (s, 1H), 8.48 (s, 1H), 8.20 (s, 1H), 8.13 (d, J = 8.8 Hz, 1H), 7.74-7.58 (m, 3H), 5.97 (dt, J = 13.1, 6.6 Hz, 1H), 3.93 (s, 3H), 1.72 (d, J = 6.3 Hz, 3H). |
| 68 | 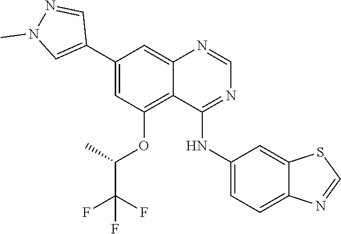 [SMILES: C[C@H](Oc1cc(-c2cnn(C)c2)cc2ncnc(Nc3ccc4ncsc4c3)c12)C(F)(F)F] | (S)-N-(7-(1-methyl-1H-pyrazol-4-yl)-5-((1,1,1-trifluoropropan-2- yl)oxy)quinazolin-4-yl)benzo[d]thiazol-6-amine LC-MS: (ES, m/z): RT = 2.512 min, LC-MS: m/z = 471 [M + 1]; ¹H NMR (500 MHz, DMSO-d6) δ 9.84 (s, 1H), 9.34 (s, 1H), 8.93 (d, J = 2.0 Hz, 1H), 8.62 (s, 1H), 8.48 (s, 1H), 8.20 (s, 1H), 8.13 (d, J = 8.8 Hz, 1H), 7.72-7.59 (m, 3H), 6.06-5.87 (m, 1H), 3.93 (s, 3H), 1.72 (d, J = 6.3 Hz, 3H). |
| 69 | 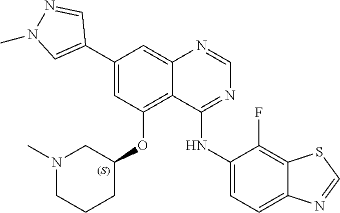 | (S)-7-fluoro-N-(7-(1-methyl-1H-pyrazol-4-yl)-5-((1- methylpiperidin-3-yl)oxy)quinazolin-4-yl)benzo[d]thiazol-6-amine LC-MS: (ES, m/z): RT = 1.866 min, LCMS: m/z = 490 [M + 1]. ¹H NMR (400 MHz, DMSO-d6) δ 10.67 (s, 1H), 9.46 (d, J = 1.5 Hz, 1H), 8.43 (d, J = 21.4 Hz, 2H), 8.15 (s, 1H), 8.02 (d, J = 8.7 Hz, 1H), 7.92 (t, J = 8.1 Hz, 1H), 7.56 (d, J = 1.5 Hz, 1H), 7.44 (d, J = 1.6 Hz, 1H), 5.20 (s, 1H), 3.91 (s, 3H), 2.96 (d, J = 11.4 Hz, 1H), 2.57 (d, J = 10.0 Hz, 1H), 2.41 (d, J = 11.6 Hz, 1H), 2.12 (d, J = 8.8 Hz, 2H), 2.07 (s, 3H), 1.77 (d, J = 12.6 Hz, 2H), 1.64 (t, J = 13.0 Hz, 1H). |
| 70 71 | 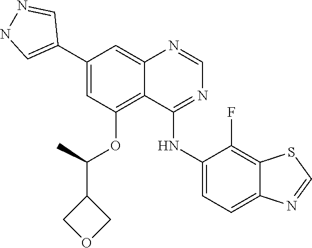 or 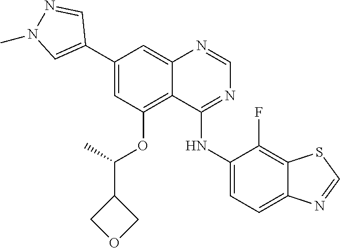 [SMILES: C[C@H](Oc1cc(-c2cnn(C)c2)cc2ncnc(Nc3ccc4ncsc4c3F)c12)C1COC1] | (S)-7-fluoro-N-(7-(1-methyl-1H-pyrazol-4-yl)-5-(1-(oxetan-3- yl)ethoxy)quinazolin-4-yl)benzo[d]thiazol-6-amine OR (R)-7- fluoro-N-(7-(1-methyl-1H-pyrazol-4-yl)-5-(1-(oxetan-3- yl)ethoxy)quinazolin-4-yl)benzo[d]thiazol-6-amine First Eluting Isomer: LC-MS: (ES, m/z): RT = 1.017 min, m/z = 477 [M + 1], chiral-HPLC : R = 2.252, ¹H NMR (400 MHz, DMSO-d6) δ 10.08 (s, 1H), 9.43 (d, J = 1.4 Hz, 1H), 8.49 (d, J = 7.4 Hz, 2H), 8.43 (t, J = 8.3 Hz, 1H), 8.19 (s, 1H), 8.00 (d, J = 8.8 Hz, 1H), 7.60 (s, 1H), 7.50 (s, 1H), 5.44 (p, J = 6.1 Hz, 1H), 4.82 (td, J = 8.1, 7.2, 2.5 Hz, 2H), 4.59 (t, J = 5.9 Hz, 1H), 4.50 (t, J = 5.9 Hz, 1H), 3.92 (s, 3H), 3.48-3.40 (m, 1H), 1.40 (d, J = 5.9 Hz, 3H). Second Eluting Isomer: LC-MS: (ES, m/z): RT = 1.018 min, m/z = 477 [M + 1], chiral-HPLC : R = 2.772, ¹H NMR (400 MHz, DMSO- d6) δ 10.08 (s, 1H), 9.43 (d, J = 1.5 Hz, 1H), 8.49 (d, J = 7.9 Hz, 2H), 8.43 (dd, J = 8.8, 7.8 Hz, 1H), 8.19 (d, J = 0.8 Hz, 1H), 8.00 (d, J = 8.8 Hz, 1H), 7.60 (d, J = 1.4 Hz, 1H), 7.50 (s, 1H), 5.47-5.40 (m, 1H), 4.86-4.77 (m, 2H), 4.59 (t, J = 5.9 Hz, 1H), 4.50 (t, J = 5.9 Hz, 1H), 3.92 (s, 3H), 3.43-3.33 (m,1H), 1.40 (d, J = 5.9 Hz, 3H). |
| 72 73 | 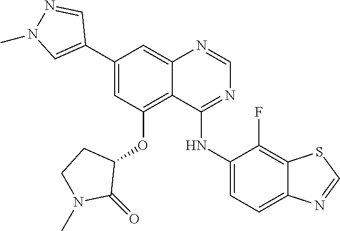 [SMILES: CN1CC[C@H](Oc2cc(-c3cnn(C)c3)cc3ncnc(Nc4ccc5ncsc5c4F)c23)C1=O] or 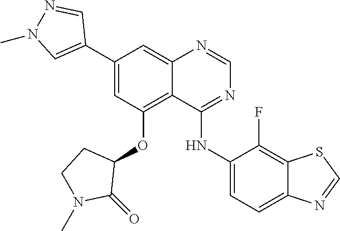 [SMILES: CN1CC[C@@H](Oc2cc(-c3cnn(C)c3)cc3ncnc(Nc4ccc5ncsc5c4F)c23)C1=O] | (R)-3-((4-((7-fluorobenzo[d]thiazol-6-yl)amino)-7-(1-methyl-1H- pyrazol-4-yl)quinazolin-5-yl)oxy)-1-methylpyrrolidin-2-one OR (S)-3-((4-((7-fluorobenzo[d]thiazol-6-yl)amino)-7-(1-methyl-1H- pyrazol-4-yl)quinazolin-5-yl)oxy)-1-methylpyrrolidin-2-one First Eluting Isomer: LC-MS: (ES, m/z): RT = 1.311 min, LCMS: m/z = 490 [M + 1], Chiral-HPLC (ES): RT = 3.122, ¹HNMR (300 MHz,DMSO-d6) δ 10.21 (s, 1H), 9.46 (d, J = 1.5 Hz, 1H), 8.47 (d, J = 1.7 Hz, 2H), 8.23-8.14 (m, 2H), 8.03 (d, J = 8.8 Hz, 1H), 7.64 (d, J = 1.4 Hz, 1H), 7.42 (d, J = 1.6 Hz, 1H), 5.43 (t, J = 8.3 Hz, 1H), 3.92 (s, 3H), 3.58-3.43 (m, 2H), 3.09-2.97 (m, 1H), 2.87 (s, 3H), 2.26 (dd, J = 12.5, 8.7 Hz, 1H). Second Eluting Isomer: LC-MS: (ES, m/z): RT = 1.308 min, LCMS: m/z = 490 [M + 1], Chiral-HPLC (ES): RT = 3.880, ¹H NMR (300 MHz, DMSO-d6) δ 10.19 (s, 1H), 9.45 (s, 1H), 8.46 (d, J = 5.0 Hz, 2H), 8.26-8.09 (m, 2H), 8.02 (d, J = 8.8 Hz, 1H), 7.63 (s, 1H), 7.41 (s, 1H), 5.42 (t, J = 8.2 Hz, 1H), 3.92 (s, 3H), 3.49 (dq, J = 17.1, 9.3 Hz, 2H), 3.02 (dt, J = 14.1, 7.4 Hz, 1H), 2.86 (s, 3H), 2.26 (dq, J = 12.7, 8.8 Hz, 1H). |
| 74 75 | 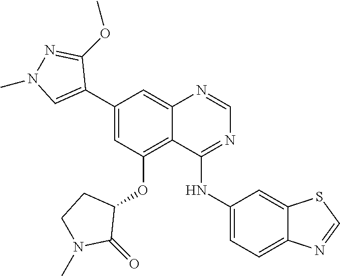 [SMILES: COc1nn(C)cc1-c1cc(O[C@H]2CCN(C)C2=O)c2c(Nc3ccc4ncsc4c3)ncnc2c1] or 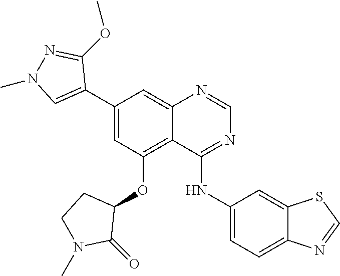 [SMILES: COc1nn(C)cc1-c1cc(O[C@@H]2CCN(C)C2=O)c2c(Nc3ccc4ncsc4c3)ncnc2c1] | (R)-3-((4-(benzo[d]thiazol-6-ylamino)-7-(3-methoxy-1-methyl-1H- pyrazol-4-yl)quinazolin-5-yl)oxy)-1-methylpyrrolidin-2-one OR (S)-3-((4-(benzo[d]thiazol-6-ylamino)-7-(3-methoxy-1-methyl-1H- pyrazol-4-yl)quinazolin-5-yl)oxy)-1-methylpyrrolidin-2-one First Eluting Isomer: LC-MS: (ES, m/z): RT = 1.438 min, LCMS: m/z = 502 [M + 1], Chiral-HPLC (ES): RT = 2.001, ¹H NMR (300 MHz,DMSO-d6) δ 10.54 (s, 1H), 9.31 (s, 1H), 9.06 (d, J = 2.1 Hz, 1H), 8.57 (s, 1H), 8.35 (s, 1H), 8.09 (d, J = 8.8 Hz, 1H), 7.95 (dd, J = 8.9, 2.2 Hz, 1H), 7.69 (d, J = 1.5 Hz, 1H), 7.38 (d, J = 1.6 Hz, 1H), 5.32 (t, J = 8.5 Hz, 1H), 4.00 (s, 3H), 3.79 (s, 3H), 3.53 (dd, J = 10.8, 3.8 Hz, 2H), 3.10-3.00 (m, 1H), 2.96 (s, 3H), 2.27-2.16 (m, 1H). Second Eluting Isomer: LC-MS: (ES, m/z): RT = 1.441 min, LCMS: m/z = 502 [M + 1], Chiral-HPLC (ES): RT = 4.702, ¹H NMR (300 MHz, DMSO-d6) δ 10.54 (s, 1H), 9.31 (s, 1H), 9.06 (d, J = 2.1 Hz, 1H), 8.57 (s, 1H), 8.35 (s, 1H), 8.09 (d, J = 8.8 Hz, 1H), 7.94 (dd, J = 8.9, 2.1 Hz, 1H), 7.69 (d, J = 1.5 Hz, 1H), 7.38 (d, J = 1.6 Hz, 1H), 5.32 (t, J = 8.5 Hz, 1H), 4.00 (s, 3H), 3.79 (s, 3H), 3.57-3.48 (m, 2H), 3.09-2.99 (m, 1H), 2.95 (s, 3H), 2.26-2.15 (m, 1H). |
| 76 | 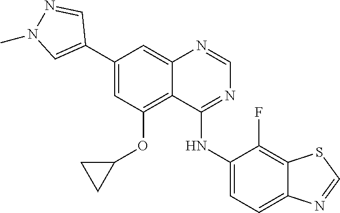 [SMILES: Cn1cc(-c2cc(OC3CC3)c3c(Nc4ccc5ncsc5c4F)ncnc3c2)cn1] | N-(5-cyclopropoxy-7-(1-methyl-1H-pyrazol-4-yl)quinazolin-4-yl)-7- fluorobenzo[d]thiazol-6-amine LC-MS: (ES, m/z): RT = 1.106 min, LCMS: m/z = 433 [M + 1]. ¹H NMR (300 MHz, DMSO-d6) δ 10.02-9.95 (m, 1H), 9.41 (d, J = 1.5 Hz, 1H), 8.81 (t, J = 8.4 Hz, 1H), 8.56 (s, 1H), 8.47 (s, 1H), 8.14 (s, 1H), 8.02 (d, J = 8.9 Hz, 1H), 7.64 (dd, J = 9.8, 1.5 Hz, 2H), 4.35 (d, J = 6.6 Hz, 1H), 3.93 (s, 3H), 1.05 (dd, J = 10.0, 3.6 Hz, 4H). |
| 77 | 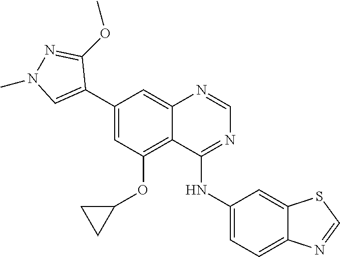 [SMILES: COc1nn(C)cc1-c1cc(OC2CC2)c2c(Nc3ccc4ncsc4c3)ncnc2c1] | N-(5-cyclopropoxy-7-(3-methoxy-1-methyl-1H-pyrazol-4- yl)quinazolin-4-yl)benzo[d]thiazol-6-amine LC-MS: (ES, m/z): RT = 1.143 min, LCMS: m/z = 445 [M + 1]. ¹H NMR (400 MHz, DMSO-d6) δ 9.92 (s, 1H), 9.31 (s, 1H), 8.87 (d, J = 2.2 Hz, 1H), 8.54 (s, 1H), 8.33 (s, 1H), 8.09 (d, J = 8.8 Hz, 1H), 7.76 (td, J = 4.7, 4.1, 2.2 Hz, 2H), 7.67 (d, J = 1.5 Hz, 1H), 4.25 (tt, J = 6.0, 3.2 Hz, 1H), 3.99 (s, 3H), 3.79 (s, 3H), 1.18-0.99 (m, 4H). |
| 78 79 | 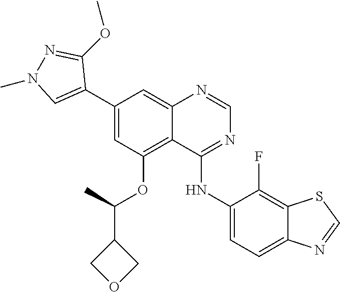 [SMILES: COc1nn(C)cc1-c1cc(O[C@H](C)C2COC2)c2c(Nc3ccc4ncsc4c3F)ncnc2c1] or 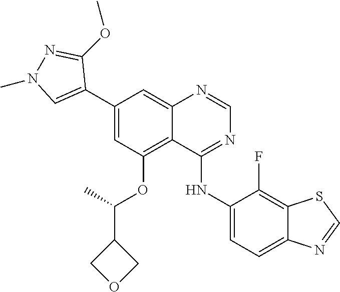 [SMILES: COc1nn(C)cc1-c1cc(O[C@@H](C)C2COC2)c2c(Nc3ccc4ncsc4c3F)ncnc2c1] | (S)-7-fluoro-N-(7-(3-methoxy-1-methyl-1H-pyrazol-4-yl)-5-(1- (oxetan-3-yl)ethoxy)quinazolin-4-yl)benzo[d]thiazol-6-amine OR (R)-7-fluoro-N-(7-(3-methoxy-1-methyl-1H-pyrazol-4-yl)-5-(1- (oxetan-3-yl)ethoxy)quinazolin-4-yl)benzo[d]thiazol-6-amine First Eluting Isomer: LC-MS: (ES, m/z): RT = 1.059 min, LCMS: m/z = 507.10 [M + 1], Chiral-HPLC (ES): RT = 1.514 min, ¹H NMR (400 MHz, DMSO-d6) δ 10.04 (s, 1H), 9.43 (d, J = 1.5 Hz, 1H), 8.46 (s, 1H), 8.43-8.34 (m, 2H), 7.99 (d, J = 8.8 Hz, 1H), 7.69 (d, J = 1.4 Hz, 1H), 7.47 (d, J = 1.7 Hz, 1H), 5.29 (p, J = 6.1 Hz, 1H), 4.82 (dd, J = 7.8, 6.4 Hz, 2H), 4.60 (t, J = 5.9 Hz, 1H), 4.50 (t, J = 5.9 Hz, 1H), 3.99 (s, 3H), 3.79 (s, 3H), 3.4(s,1H), 1.41 (d, J = 6.0 Hz, 3H). Second Eluting Isomer: LC-MS: (ES, m/z): RT = 1.089 min, LCMS: m/z = 507.10 [M + 1], Chiral-HPLC (ES): RT = 1.911 min, ¹H NMR (400 MHz, DMSO-d6) δ 10.03 (s, 1H), 9.42 (d, J = 1.5 Hz, 1H), 8.45 (s, 1H), 8.42-8.33 (m, 2H), 7.99 (d, J = 8.8 Hz, 1H), 7.68 (d, J = 1.4 Hz, 1H), 7.47 (d, J = 1.6 Hz, 1H), 5.29 (p, J = 6.1 Hz, 1H), 4.82 (dd, J = 7.8, 6.4 Hz, 2H), 4.60 (t, J = 5.9 Hz, 1H), 4.50 (t, J = 5.9 Hz, 1H), 3.99 (s, 3H), 3.79 (s, 3H), 3.4(s, 1H), 1.41 (d, J = 6.1 Hz, 3H). |
| 80 81 | 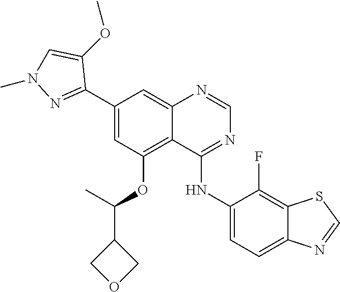 [SMILES: COc1cn(C)nc1-c1cc(O[C@H](C)C2COC2)c2c(Nc3ccc4ncsc4c3F)ncnc2c1] or 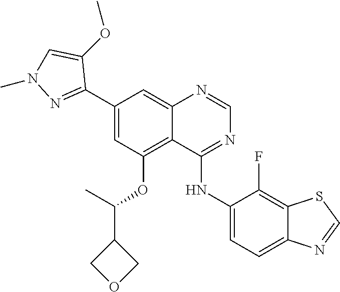 [SMILES: COc1cn(C)nc1-c1cc(O[C@@H](C)C2COC2)c2c(Nc3ccc4ncsc4c3F)ncnc2c1] | (S)-7-fluoro-N-(7-(4-methoxy-1-methyl-1H-pyrazol-3-yl)-5-(1- (oxetan-3-yl)ethoxy)quinazolin-4-yl)benzo[d]thiazol-6-amine OR (R)-7-fluoro-N-(7-(4-methoxy-1-methyl-1H-pyrazol-3-yl)-5-(1- (oxetan-3-yl)ethoxy)quinazolin-4-yl)benzo[d]thiazol-6-amine First Eluting Isomer: LC-MS: (ES, m/z): RT = 1.091 min, LCMS: m/z = 507 [M + 1], Chiral-HPLC: RT = 2.834 min, ¹H NMR (300 MHz, DMSO-d6) δ 10.04 (s, 1H), 9.42 (d, J = 1.6 Hz, 1H), 8.46 (s, 1H), 8.34-8.22 (m, 1H), 7.98 (d, J = 8.8 Hz, 1H), 7.89 (d, J = 1.3 Hz, 1H), 7.71 (s, 1H), 7.68 (s, 1H), 5.18 (t, J = 6.1 Hz, 1H), 4.79 (q, J = 6.6 Hz, 2H), 4.55 (dt, J = 19.1, 5.8 Hz, 2H), 3.86 (d, J = 8.2 Hz, 6H), 3.42-3.33 (m, 1H), 1.41 (d, J = 6.0 Hz, 3H). Second Eluting Isomer: LC-MS: (ES, m/z): RT = 1.087 min, LCMS: m/z = 507 [M + 1], Chiral-HPLC: RT = 4.162 min, ¹H NMR (300 MHz, DMSO-d6) δ 10.04 (s, 1H), 9.42 (d, J = 1.6 Hz, 1H), 8.46 (s, 1H), 8.34-8.22 (m, 1H), 7.98 (d, J = 8.8 Hz, 1H), 7.89 (d, J = 1.3 Hz, 1H), 7.71 (s, 1H), 7.68 (s, 1H), 5.18 (t, J = 6.1 Hz, 1H), 4.79 (q, J = 6.6 Hz, 2H), 4.55 (dt, J = 19.1, 5.8 Hz, 2H), 3.86 (d, J = 8.2 Hz, 6H), 3.42-3.33 (m, 1H), 1.41 (d, J = 6.0 Hz, 3H). |
| 82 83 | 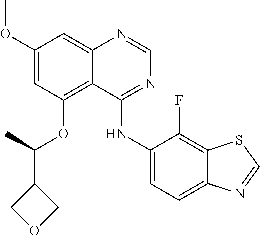 [SMILES: COc1cc(O[C@H](C)C2COC2)c2c(Nc3ccc4ncsc4c3F)ncnc2c1] or 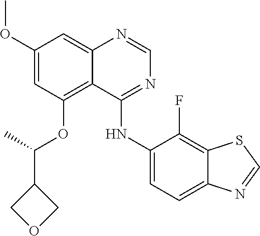 [SMILES: COc1cc(O[C@@H](C)C2COC2)c2c(Nc3ccc4ncsc4c3F)ncnc2c1] | (S)-7-fluoro-N-(7-methoxy-5-(1-(oxetan-3-yl)ethoxy)quinazolin-4- yl)benzo[d]thiazol-6-amine OR (R)-7-fluoro-N-(7-methoxy-5-(1- (oxetan-3-yl)ethoxy)quinazolin-4-yl)benzo[d]thiazol-6-amine First Eluting Isomer: LC-MS: (ES, m/z): RT = 1.065 min, LCMS: m/z = 427 [M + 1], chiral-HPLC: (ES, m/z): R = 1.472 min, ¹H NMR (300 MHz, DMSO-d6) δ 9.94 (s, 1H), 9.43 (d, J = 1.6 Hz, 1H), 8.44 (s, 1H), 8.32 (t, J = 8.3 Hz, 1H), 7.99 (d, J = 8.9 Hz, 1H), 6.93-6.83 (m, 2H), 5.27-5.17 (m, 1H), 4.85-4.74 (m, 2H), 4.56 (t, J = 5.9 Hz, 1H), 4.47 (t, J = 5.9 Hz, 1H), 3.94 (s, 3H), 3.41-3.36 (m, 1H), 1.37 (d, J = 5.9 Hz, 3H). Second Eluting Isomer: LC-MS: (ES, m/z): RT = 1.058 min, LCMS: m/z = 427 [M + 1], chiral-HPLC: (ES, m/z): R = 2.089 min, ¹H NMR (300 MHz, DMSO-d6) δ 9.94 (s, 1H), 9.43 (d, J = 1.6 Hz, 1H), 8.44 (s, 1H), 8.38-8.27 (m, 1H), 7.99 (d, J = 8.8 Hz, 1H), 6.93-6.83 (m, 2H), 5.22 (t, J = 6.2 Hz, 1H), 4.85-4.73 (m, 2H), 4.56 (t, J = 5.9 Hz, 1H), 4.47 (t, J = 5.8 Hz, 1H), 3.94 (s, 3H), 3.42-3.35 (m, 1H), 1.37 (d, J = 6.0 Hz, 3H). |
| 84 85 | 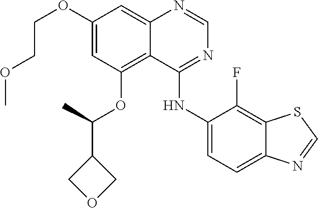 [SMILES: COCCOc1cc(O[C@H](C)C2COC2)c2c(Nc3ccc4ncsc4c3F)ncnc2c1] or 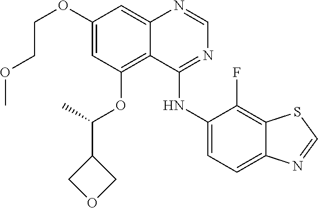 [SMILES: COCCOc1cc(O[C@@H](C)C2COC2)c2c(Nc3ccc4ncsc4c3F)ncnc2c1] | (S)-7-fluoro-N-(7-(2-methoxyethoxy)-5-(1-(oxetan-3- yl)ethoxy)quinazolin-4-yl)benzo[d]thiazol-6-amine OR (R)-7- fluoro-N-(7-(2-methoxyethoxy)-5-(1-(oxetan-3- yl)ethoxy)quinazolin-4-yl)benzo[d]thiazol-6-amine First Eluting Isomer: LC-MS: (ES, m/z): RT-0.870 min, LCMS: m/z = 471 [M + 1], chiral-HPLC: (ES, m/z): R = 2.209 min, ¹H NMR (300 MHz, DMSO-d6) δ 9.94 (s, 1H), 9.43 (d, J = 1.6 Hz, 1H), 8.44 (s, 1H), 8.40-8.28 (m, 1H), 7.99 (d, J = 8.8 Hz, 1H), 6.89 (dd, J = 19.5, 2.2 Hz, 2H), 5.31-5.20 (m, 1H), 4.84-4.73 (m, 2H), 4.52 (dt, J = 26.6, 5.9 Hz, 2H), 4.29 (t, J = 4.5 Hz, 2H), 3.74 (t, J = 4.5 Hz, 2H), 3.41-3.37 (m, 4H), 1.37 (d, J = 6.0 Hz, 3H). Second Eluting Isomer: LC-MS: (ES, m/z): RT-0.867 min, LCMS: m/z = 471 [M + 1], chiral-HPLC: (ES, m/z): R = 3.251 min, ¹H NMR (300 MHz, DMSO-d6) δ 9.94 (s, 1H), 9.43 (d, J = 1.5 Hz, 1H), 8.44 (s, 1H), 8.40-8.28 (m, 1H), 7.99 (d, J = 8.8 Hz, 1H), 6.89 (dd, J = 19.4, 2.2 Hz, 2H), 5.25 (t, J = 6.2 Hz, 1H), 4.85-4.73 (m, 2H), 4.52 (dt, J = 26.7, 5.9 Hz, 2H), 4.33-4.24 (m, 2H), 3.74 (t, J = 4.4 Hz, 2H), 3.42-3.38 (m, 4H), 1.37 (d, J = 6.0 Hz, 3H). |
| 86 87 | 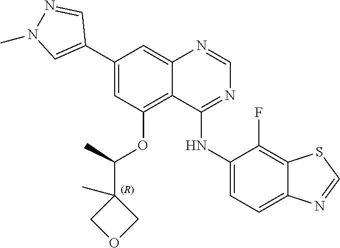 or 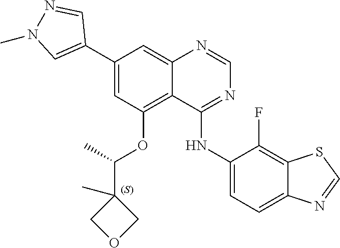 | (S)-7-fluoro-N-(7-(1-methyl-1H-pyrazol-4-yl)-5-(1-(3- methyloxetan-3-yl)ethoxy)quinazolin-4-yl)benzo[d]thiazol-6-amine OR (R)-7-fluoro-N-(7-(1-methyl-1H-pyrazol-4-yl)-5-(1-(3- methyloxetan-3-yl)ethoxy)quinazolin-4-yl)benzo[d]thiazol-6-amine First Eluting Isomer: LC-MS: (ES, m/z): RT = 0.875 min, LCMS: m/z = 491 [M + 1], chiral-HPLC: (ES, m/z): R = 4.634 min, ¹H NMR (400 MHz, DMSO-d6) δ 9.99 (d, J = 2.8 Hz, 1H), 9.46 (d, J = 1.5 Hz, 1H), 8.51 (s, 1H), 8.41 (s, 1H), 8.19 (d, J = 0.8 Hz, 1H), 7.98 (d, J = 8.7 Hz, 1H), 7.94-7.84 (m, 1H), 7.58 (d, J = 1.4 Hz, 1H), 7.44 (d, J = 1.5 Hz, 1H), 5.20 (q, J = 5.9 Hz, 1H), 4.73 (d, J = 6.3 Hz, 1H), 4.64 (d, J = 6.0 Hz, 1H), 4.50-4.41 (m, 2H), 3.93 (s, 3H), 1.42- 1.34 (m, 6H). Second Eluting Isomer: LC-MS: (ES, m/z): RT = 1.154 min, LCMS: m/z = 491 [M + 1], chiral-HPLC: (ES, m/z): R = 6.472 min, ¹H NMR (300 MHz, DMSO-d6) δ 9.97 (s, 1H), 9.46 (d, J = 1.5 Hz, 1H), 8.51 (s, 1H), 8.41 (d, J = 1.4 Hz, 1H), 8.19 (s, 1H), 7.98 (d, J = 8.7 Hz, 1H), 7.89 (dt, J = 8.7, 6.6 Hz, 1H), 7.59 (d, J = 1.3 Hz, 1H), 7.44 (d, J = 1.5 Hz, 1H), 5.20 (q, J = 5.9 Hz, 1H), 4.73 (d, J = 6.3 Hz, 1H), 4.64 (d, J = 6.0 Hz, 1H), 4.46 (t, J = 6.4 Hz, 2H), 3.93 (s, 3H), 1.43-1.33 (m, 6H). |
| 88 89 | 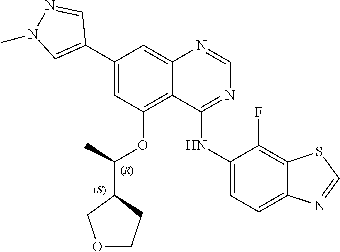 or 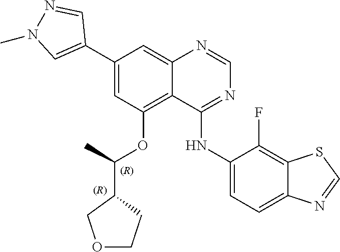 | 7-fluoro-N-(7-(1-methyl-1H-pyrazol-4-yl)-5-((R)-1-((R)- tetrahydrofuran-3-yl)ethoxy)quinazolin-4-yl)benzo[d]thiazol-6- amine OR 7-fluoro-N-(7-(1-methyl-1H-pyrazol-4-yl)-5-((R)-1- ((S)-tetrahydrofuran-3-yl)ethoxy)quinazolin-4-yl)benzo[d]thiazol-6- amine First Eluting Isomer: LC-MS: (ES, m/z): RT = 1.085 min, LCMS: m/z = 491 [M + 1], CHIRAL-HPLC: RT = 5.095 min, ¹H NMR (400 MHz, DMSO-d6) δ 10.07 (s, 1H), 9.42 (d, J = 1.5 Hz, 1H), 8.47 (s, 2H), 8.50-8.41 (m, 1H), 8.15 (s, 1H), 8.00 (d, J = 8.8 Hz, 1H), 7.56 (d, J = 1.4 Hz, 1H), 7.41 (d, J = 1.6 Hz, 1H), 5.03 (q, J = 6.2 Hz, 1H), 3.90 (s, 3H), 3.79 (dd, J = 6.3, 2.0 Hz, 2H), 3.60 (q, J = 8.0 Hz, 1H), 2.75 (m, 1H), 2.18-2.06 (m, 1H), 1.74 (dq, J = 14.8, 7.8, 7.8, 7.8 Hz, 1H), 1.44 (d, J = 6.0 Hz, 3H). Second Eluting Isomer: LC-MS: (ES, m/z): RT = 1.088 min, LCMS: m/z = 491 [M + 1]CHIRAL-HPLC: RT = 5.902 min, ¹H NMR (400 MHz, DMSO-d6) δ 10.16 (s, 1H), 9.42 (d, J = 1.6 Hz, 1H), 8.60 (t, J = 8.4 Hz, 1H), 8.50 (d, J = 14.3 Hz, 2H), 8.16 (d, J = 0.7 Hz, 1H), 8.02 (d, J = 8.9 Hz, 1H), 7.57 (d, J = 1.4 Hz, 1H), 7.44 (d, J = 1.6 Hz, 1H), 5.07-4.99 (m, 1H), 3.90 (s, 3H), 3.84 (t, J = 8.2 Hz, 1H), 3.75 (m, 1H), 3.67-3.56 (m, 2H), 2.79 (m, 1H), 2.18-2.06 (m, 1H), 1.88-1.74 (m, 1H), 1.41 (d, J = 5.9 Hz, 3H). |
| 90 | 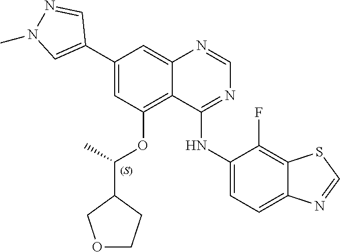 | 7-fluoro-N-(7-(1-methyl-1H-pyrazol-4-yl)-5-((1S)-1- (tetrahydrofuran-3-yl)ethoxy)quinazolin-4-yl)benzo[d]thiazol-6- amine LC-MS: (ES, m/z): RT = 1.370 min, LCMS: m/z = 491 [M + 1]. ¹H NMR (400 MHz, DMSO-d6) δ 10.11 (d, J = 31.6 Hz, 1H), 9.43 (t, J = 1.8 Hz, 1H), 8.65 (d, J = 8.5 Hz, 1H), 8.57-8.43 (m, 2H), 8.17 (d, J = 2.7 Hz, 1H), 8.02 (dd, J = 8.9, 5.1 Hz, 1H), 7.58 (dd, J = 5.0, 1.4 Hz, 1H), 7.46-7.35 (m, 1H), 5.05 (t, J = 7.1 Hz, 1H), 3.91 (s, 3H), 3.85 (d, J = 8.0 Hz, 2H), 3.69-3.57 (m, 2H), 2.87-2.70 (m, 1H), 2.14 (d, J = 7.5 Hz, 1H), 1.92-1.67 (m, 1H), 1.45 (dd, J = 13.1, 6.0 Hz, 3H). |
| 91 | 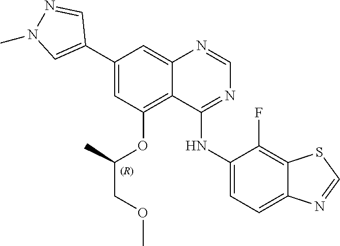 | (R)-7-fluoro-N-(5-((1-methoxypropan-2-yl)oxy)-7-(1-methyl-1H- pyrazol-4-yl)quinazolin-4-yl)benzo[d]thiazol-6-amine LC-MS: (ES, m/z): RT = 1.081 min, LCMS: m/z = 465 [M + 1], ¹H NMR (300 MHz, DMSO-d6) δ 10.24 (s, 1H), 9.42 (d, J = 1.5 Hz, 1H), 8.48 (d, J = 8.6 Hz, 3H), 8.15 (s, 1H), 8.02 (d, J = 8.8 Hz, 1H), 7.58 (d, J = 1.4 Hz, 1H), 7.46 (d, J = 1.5 Hz, 1H), 5.24 (d, J = 6.0 Hz, 1H), 3.91 (s, 3H), 3.76-3.68 (m, 2H), 3.26 (s, 3H), 1.46 (d, J = 6.2 Hz, 3H). |
| 92 93 | 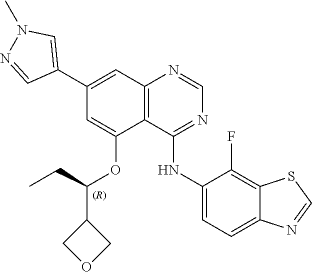 or 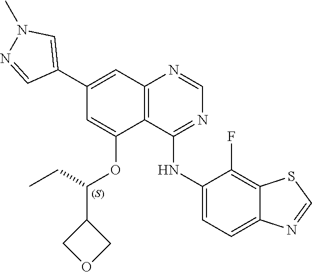 | (S)-7-fluoro-N-(7-(1-methyl-1H-pyrazol-4-yl)-5-(1-(oxetan-3- yl)propoxy)quinazolin-4-yl)benzo[d]thiazol-6-amine OR (R)-7- fluoro-N-(7-(1-methyl-1H-pyrazol-4-yl)-5-(1-(oxetan-3- yl)propoxy)quinazolin-4-yl)benzo[d]thiazol-6-amine First Eluting Isomer: LC-MS: (ES, m/z): RT = 0.864 min, LCMS: m/z = 491.1[M + 1], Chiral HPLC RT: 2.773, ¹H NMR (400 MHz, DMSO-d6) δ 10.16 (s, 1H), 9.43 (d, J = 1.5 Hz, 1H), 8.63-8.51 (m, 2H), 8.50 (s, 1H), 8.19 (s, 1H), 8.02 (d, J = 8.9 Hz, 1H), 7.60 (d, J = 1.4 Hz, 1H), 7.53 (d, J = 1.7 Hz, 1H), 5.43 (d, J = 6.8 Hz, 1H), 4.77 (td, J = 8.3, 6.3 Hz, 2H), 4.59 (t, J = 6.2 Hz, 1H), 4.52 (t, J = 6.2 Hz, 1H), 3.93 (s, 3H), 3.59 (q, J = 6.9 Hz, 1H), 1.82 (q, J = 7.1 Hz, 2H), 0.93 (t, J = 7.4 Hz, 3H) Second Eluting Isomer: LC-MS: (ES, m/z): RT = 0.866 min, LCMS: m/z = 491 [M + 1], Chiral HPLC: RT = 3.321, 1H NMR (300 MHz, DMSO-d6) δ 10.14 (s, 1H), 9.44 (d, J = 1.6 Hz, 1H), 8.58 (dd, J = 8.9, 7.9 Hz, 1H), 8.51 (d, J = 3.3 Hz, 2H), 8.20 (s, 1H), 8.02 (d, J = 8.8 Hz, 1H), 7.61 (d, J = 1.4 Hz, 1H), 7.53 (s, 1H), 5.47-5.37 (m, 1H), 4.84-4.71 (m, 2H), 4.60 (t, J = 6.2 Hz, 1H), 4.53 (t, J = 6.1 Hz, 1H), 3.93 (s, 3H), 3.60 (q, J = 7.0 Hz, 1H), 1.84 (p, J = 7.2 Hz, 2H), 0.94 (t, J = 7.4 Hz, 3H). |
| 94 95 | 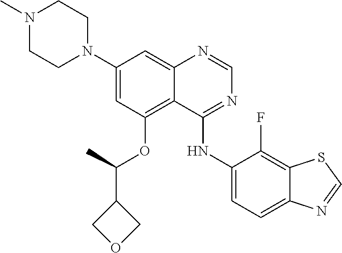 [SMILES: C[C@@H](Oc1cc(N2CCN(C)CC2)cc2ncnc(Nc3ccc4ncsc4c3F)c12)C1COC1] or 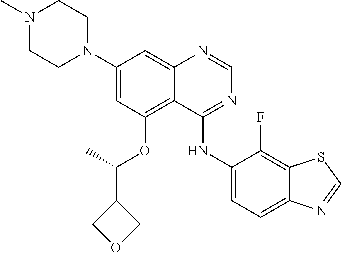 [SMILES: C[C@H](Oc1cc(N2CCN(C)CC2)cc2ncnc(Nc3ccc4ncsc4c3F)c12)C1COC1] | (S)-7-fluoro-N-(7-(4-methylpiperazin-1-yl)-5-(1-(oxetan-3- yl)ethoxy)quinazolin-4-yl)benzo[d]thiazol-6-amine. OR (R)-7- fluoro-N-(7-(4-methylpiperazin-1-yl)-5-(1-(oxetan-3- yl)ethoxy)quinazolin-4-yl)benzo[d]thiazol-6-amine First Eluting Isomer: LC-MS: (ES, m/z): RT = 0.667 min, LCMS: m/z = 495 [M + 1], Chiral-HPLC (ES): RT = 2.084, ¹H NMR (300 MHz,DMSO-d6) δ 9.90 (s, 1H), 9.41 (d, J = 1.6 Hz, 1H), 8.56-8.44 (m, 1H), 8.36 (s, 1H), 7.98 (d, J = 8.9 Hz, 1H), 6.96 (s, 1H), 6.68 (s, 1H), 5.42-5.31 (m, 1H), 4.79 (t, J = 6.8 Hz, 2H), 4.56 (t, J = 6.0 Hz, 1H), 4.47 (t, J = 5.9 Hz, 1H), 3.42 (s, 6H), 3.24 (m, 1H), 2.47 (m, 2H), 2.26 (s, 3H), 1.34 (d, J = 5.9 Hz, 3H). Second Eluting Isomer: LC-MS: (ES, m/z): RT = 0.501 min, LCMS: m/z = 495 [M + 1], Chiral-HPLC (ES): RT = 3.143, ¹H NMR (300 MHz, DMSO-d6) δ 9.90 (s, 1H), 9.41 (d, J = 1.6 Hz, 1H), 8.50 (t, J = 8.4 Hz, 1H), 8.36 (s, 1H), 7.98 (d, J = 8.9 Hz, 1H), 6.96 (s, 1H), 6.68 (s, 1H), 5.41-5.31 (m, 1H), 4.79 (t, J = 7.1 Hz, 2H), 4.56 (t, J = 6.0 Hz, 1H), 4.47 (t, J = 5.9 Hz, 1H), 3.42 (s, 6H), 3.24 (s, 6H), 2.48 (m, 2H), 2.26 (s, 3H), 1.34 (d, J = 5.9 Hz, 3H). |
| 96 97 | 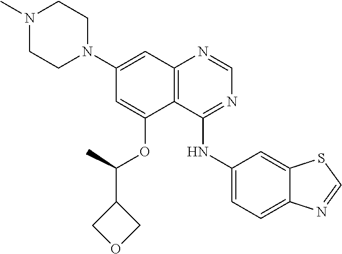 [SMILES: C[C@@H](Oc1cc(N2CCN(C)CC2)cc2ncnc(Nc3ccc4ncsc4c3)c12)C1COC1] or 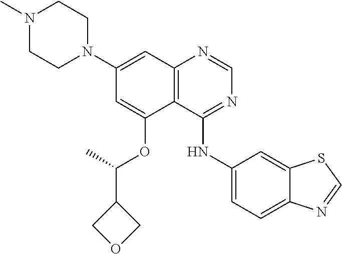 [SMILES: C[C@H](Oc1cc(N2CCN(C)CC2)cc2ncnc(Nc3ccc4ncsc4c3)c12)C1COC1] | (R)-N-(7-(4-methylpiperazin-1-yl)-5-(1-(oxetan-3- yl)ethoxy)quinazolin-4-yl)benzo[d]thiazol-6-amine OR (S)-N-(7- (4-methylpiperazin-1-yl)-5-(1-(oxetan-3-yl)ethoxy)quinazolin-4- yl)benzo[d]thiazol-6-amine First Eluting Isomer: LC-MS: (ES, m/z): RT = 0.957 min, LCMS: m/z = 477 [M + 1], CHIRAL-HPLC: RT = 1.339 min, ¹H NMR (300 MHz, DMSO-d6) δ 9.94 (s, 1H), 9.28 (s, 1H), 8.74 (d, J = 2.1 Hz, 1H), 8.38 (s, 1H), 8.04 (d, J = 8.8 Hz, 1H), 7.73 (dd, J = 8.8, 2.2 Hz, 1H), 6.91 (d, J = 2.2 Hz, 1H), 6.65 (d, J = 2.1 Hz, 1H), 5.31 (p, J = 6.1 Hz, 1H), 4.79 (dt, J = 8.0, 6.1 Hz, 2H), 4.58 (t, J = 6.0 Hz, 1H), 4.51 (t, J = 6.0 Hz, 1H), 3.48 (q, J = 7.0 Hz, 1H), 3.37 (s, 4H), 2.50 (s, 1H), 2.45 (s, 3H), 2.23 (s, 3H), 1.34 (d, J = 5.9 Hz, 3H). Second Eluting Isomer: LC-MS: (ES, m/z): RT = 0.952 min, LCMS: m/z = 477 [M + 1], CHIRAL-HPLC: RT = 1.720 min, ¹H NMR (300 MHz, DMSO-d6) δ 9.94 (s, 1H), 9.28 (s, 1H), 8.74 (d, J = 2.1 Hz, 1H), 8.38 (s, 1H), 8.04 (d, J = 8.8 Hz, 1H), 7.73 (dd, J = 8.8, 2.2 Hz, 1H), 6.91 (d, J = 2.2 Hz, 1H), 6.65 (d, J = 2.1 Hz, 1H), 5.31 (p, J = 6.1 Hz, 1H), 4.79 (dt, J = 8.0, 6.1 Hz, 2H), 4.58 (t, J = 6.0 Hz, 1H), 4.51 (t, J = 6.0 Hz, 1H), 3.48 (q, J = 7.0 Hz, 1H), 3.37 (s, 4H), 2.50 (s, 1H), 2.45 (s, 3H), 2.23 (s, 3H), 1.34 (d, J = 5.9 Hz, 3H). |
| 98 99 | 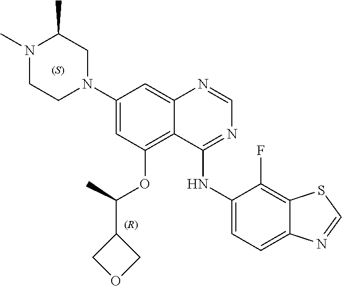 or 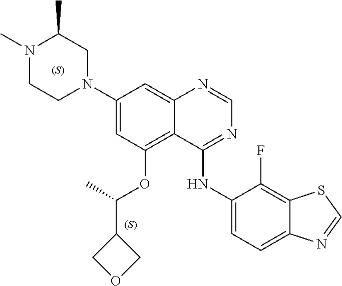 | N-(7-((S)-3,4-dimethylpiperazin-1-yl)-5-((S)-1-(oxetan-3- yl)ethoxy)quinazolin-4-yl)-7-fluorobenzo[d]thiazol-6-amine OR N- (7-((S)-3,4-dimethylpiperazin-1-yl)-5-((R)-1-(oxetan-3- yl)ethoxy)quinazolin-4-yl)-7-fluorobenzo[d]thiazol-6-amine First Eluting Isomer: LC-MS: (ES, m/z): RT = 0.681 min, LCMS: m/z = 509 [M + 1], Chiral-HPLC (ES): RT = 3.093, ¹H NMR (300 MHz,DMSO-d6) δ 9.89 (s, 1H), 9.41 (d, J = 1.6 Hz, 1H), 8.55-8.44 (m, 1H), 8.35 (s, 1H), 7.98 (d, J = 8.8 Hz, 1H), 6.95 (s, 1H), 6.68 (d, J = 2.0 Hz, 1H), 5.36 (t, J = 6.5 Hz, 1H), 4.79 (t, J = 7.0 Hz, 2H), 4.56 (t, J = 5.9 Hz, 1H), 4.48 (t, J = 5.9 Hz, 1H), 3.87 (t, J = 13.2 Hz, 2H), 3.41 (s, 1H), 2.92 (dd, J = 34.9, 12.2 Hz, 2H), 2.66-2.53 (m, 1H), 2.24 (s, 4H), 2.15 (s, 1H), 1.35 (d, J = 5.9 Hz, 3H), 1.11 (d, J = 6.2 Hz, 3H). Second Eluting Isomer: LC-MS: (ES, m/z): RT = 0.683 min, LCMS: m/z = 509 [M + 1], Chiral-HPLC (ES): RT = 3.980, ¹H NMR (300 MHz, DMSO-d6) δ 9.89 (s, 1H), 9.41 (d, J = 1.6 Hz, 1H), 8.55- 8.44 (m, 1H), 8.35 (s, 1H), 7.98 (d, J = 8.8 Hz, 1H), 6.95 (s, 1H), 6.68 (d, J = 2.0 Hz, 1H), 5.36 (t, J = 6.5 Hz, 1H), 4.79 (t, J = 7.0 Hz, 2H), 4.56 (t, J = 5.9 Hz, 1H), 4.48 (t, J = 5.9 Hz, 1H), 3.87 (t, J = 13.2 Hz, 2H), 3.41 (s, 1H), 2.92 (dd, J = 34.9, 12.2 Hz, 2H), 2.66- 2.53 (m, 1H), 2.24 (s, 4H), 2.15 (s, 1H), 1.35 (d, J = 5.9 Hz, 3H), 1.11 (d, J = 6.2 Hz, 3H). |
| 100 101 | 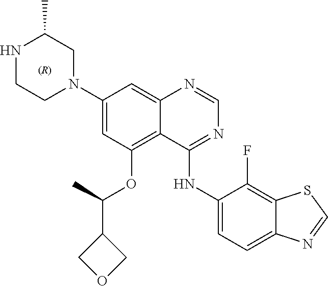 or 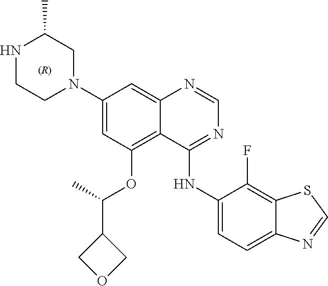 | 7-fluoro-N-(7-((R)-3-methylpiperazin-1-yl)-5-((S)-1-(oxetan-3- yl)ethoxy)quinazolin-4-yl)benzo[d]thiazol-6-amine OR 7-fluoro-N- (7-((R)-3-methylpiperazin-1-yl)-5-((R)-1-(oxetan-3- yl)ethoxy)quinazolin-4-yl)benzo[d]thiazol-6-amine First Eluting Isomer: LC-MS: (ES, m/z): RT = 0.609 min, LCMS: m/z = 495 [M + 1], HPLC: RT = 2.275 min; ¹H NMR (400 MHz, DMSO-d6) δ 9.90 (d, J = 1.5 Hz, 1H), 9.42 (d, J = 1.5 Hz, 1H), 8.48 (dt, J = 14.3, 8.3 Hz, 1H), 8.39 (s, 1H), 7.99 (d, J = 8.8 Hz, 1H), 6.99 (d, J = 2.3 Hz, 1H), 6.77 (d, J = 2.1 Hz, 1H), 5.39-5.28 (m, 1H), 4.81 (ddd, J = 7.7, 6.4, 4.2 Hz, 2H), 4.56 (t, J = 5.9 Hz, 1H), 4.47 (t, J = 5.9 Hz, 1H), 4.09 (t, J = 14.7 Hz, 2H), 3.41 (d, J = 6.9 Hz, 3H), 3.10 (t, J = 10.7 Hz, 2H), 2.82 (t, J = 12.1 Hz, 1H), 1.36 (d, J = 6.0 Hz, 3H), 1.28 (dd, J = 6.5, 1.9 Hz, 3H). Second Eluting Isomer: LC-MS: (ES, m/z): RT = 0.779 min, LCMS: m/z = 495 [M + 1], HPLC: RT = 3.214 min; ¹H NMR (400 MHz, DMSO-d6) δ 9.91 (s, 1H), 9.42 (d, J = 1.6 Hz, 1H), 8.49 (dt, J = 14.3, 8.3 Hz, 1H), 8.44 (s, 1H), 8.39 (s, 1H), 7.99 (d, J = 8.8 Hz, 1H), 7.00 (d, J = 2.3 Hz, 1H), 6.78 (d, J = 2.2 Hz, 1H), 5.36 (t, J = 6.5 Hz, 1H), 4.85-4.76 (m, 2H), 4.56 (t, J = 5.9 Hz, 1H), 4.47 (t, J = 5.9 Hz, 1H), 4.11 (dd, J = 11.1, 5.4 Hz, 2H), 3.40 (dd, J = 12.4, 4.3 Hz, 3H), 3.13 (h, J = 11.9, 11.3 Hz, 2H), 2.87 (dd, J = 13.4, 10.4 Hz, 1H), 1.36 (d, J = 5.9 Hz, 3H), 1.29 (d, J = 6.4 Hz, 3H). |
| 102 103 | 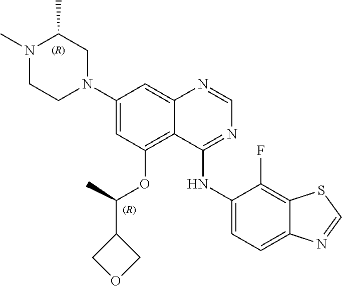 or 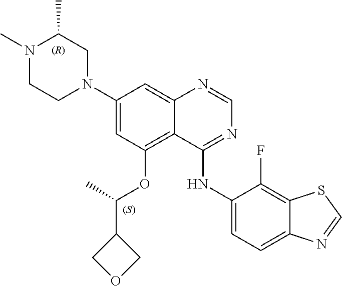 | N-(7-((R)-3,4-dimethylpiperazin-1-yl)-5-((R)-1-(oxetan-3- yl)ethoxy)quinazolin-4-yl)-7-fluorobenzo[d]thiazol-6-amine OR N- (7-((R)-3,4-dimethylpiperazin-1-yl)-5-((S)-1-(oxetan-3- yl)ethoxy)quinazolin-4-yl)-7-fluorobenzo[d]thiazol-6-amine First Eluting isomer: LC-MS: (ES, m/z): RT = 0.702 min, LCMS: m/z = 509 [M + 1], HPLC: RT = 1.729 min, ¹H NMR (300 MHz, DMSO-d6) δ 9.89 (s, 1H), 9.40 (d, J = 1.6 Hz, 1H), 8.47 (t, J = 8.4 Hz, 1H), 8.35 (s, 1H), 7.98 (d, J = 8.9 Hz, 1H), 6.98-6.91 (m, 1H), 6.68 (d, J = 2.1 Hz, 1H), 5.41-5.31 (m, 1H), 4.85-4.74 (m, 2H), 4.56 (t, J = 5.9 Hz, 1H), 4.47 (t, J = 5.9 Hz, 1H), 3.88 (t, J = 13.0 Hz, 2H), 3.55-3.45 (s, 1H) 2.88 (d, J = 12.7 Hz, 2H), 2.59 (t, J = 11.1 Hz, 1H), 2.25-2.18 (m, 5H), 1.34 (d, J = 5.9 Hz, 3H), 1.23 (s, 1H), 1.11 (d, J = 6.1 Hz, 3H). Second Eluting isomer: LC-MS: (ES, m/z): RT = 0.684 min, LCMS: m/z = 509 [M + 1], HPLC: RT = 2.173 min, ¹H NMR (300 MHz, DMSO-d6) δ 9.89 (s, 1H), 9.41 (d, J = 1.6 Hz, 1H), 8.58-8.43 (m, 1H), 8.35 (s, 1H), 7.98 (d, J = 8.9 Hz, 1H), 6.95 (d, J = 2.2 Hz, 1H), 6.68 (d, J = 2.1 Hz, 1H), 5.36 (t, J = 6.5 Hz, 1H), 4.90-4.70 (m, 2H), 4.52 (dt, J = 24.5, 6.0 Hz, 2H), 3.87 (t, J = 13.3 Hz, 2H), 3.49- 3.38 (s, 1H), 3.07-2.83 (m, 2H), 2.62 (d, J = 10.9 Hz, 1H), 2.25 (s, 5H), 1.35 (d, J = 5.9 Hz, 3H), 1.23 (s, 1H), 1.11 (d, J = 6.2 Hz, 3H). |
| 104 105 | 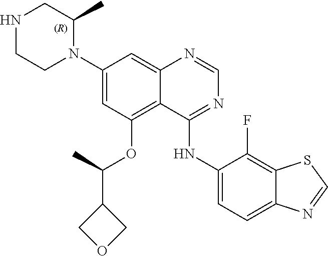 or 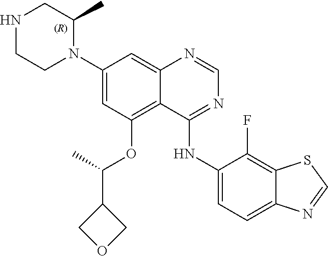 | 7-fluoro-N-(7-((R)-2-methylpiperazin-1-yl)-5-((S)-1-(oxetan-3- yl)ethoxy)quinazolin-4-yl)benzo[d]thiazol-6-amine OR 7-fluoro-N- (7-((R)-2-methylpiperazin-1-yl)-5-((R)-1-(oxetan-3- yl)ethoxy)quinazolin-4-yl)benzo[d]thiazol-6-amine First Eluting Isomer: LC-MS: (ES, m/z): RT = 0.789 min, LCMS: m/z = 495 [M + 1], HPLC: RT = 1.936 min; ¹H NMR (400 MHz, DMSO-d6) δ 9.89-9.84 (m, 1H), 9.40 (d, J = 1.5 Hz, 1H), 8.48 (t, J = 8.4 Hz, 1H), 8.33 (s, 1H), 7.98 (d, J = 8.9 Hz, 1H), 6.90 (d, J = 2.3 Hz, 1H), 6.60 (d, J = 2.2 Hz, 1H), 5.35 (p, J = 6.2 Hz, 1H), 4.83- 4.75 (m, 2H), 4.55-4.47 (m,2H), 4.26 (s, 1H), 3.58-3.45 (m, 3H) 3.08-2.83 (m, 4H), 2.77-2.67 (m, 1H), 1.34 (d, J = 5.9 Hz, 3H), (d, J = 6.5 Hz, 3H). Second Eluting Isomer: LC-MS: (ES, m/z): RT = 0.686 min, LCMS: m/z = 495 [M + 1], HPLC: RT = 2.441 min; ¹H NMR (400 MHz, DMSO-d6) δ 9.91-9.83 (m, 1H), 9.41 (d, J = 1.5 Hz, 1H), 8.58-8.44 (m, 1H), 8.34 (s, 1H), 7.98 (d, J = 8.9 Hz, 1H), 6.90 (d, J = 2.3 Hz, 1H), 6.61 (d, J = 2.1 Hz, 1H), 5.34 (p, J = 6.1 Hz, 1H), 4.78 (ddd, J = 8.0, 6.3, 2.2 Hz, 2H), 4.56 (t, J = 6.0 Hz, 1H), 4.48 (t, J = 6.0 Hz, 1H), 4.23 (dd, J = 13.6, 7.1 Hz, 1H), 3.63-3.54 (m, 1H), 3.53-3.46 (s, 1H), 3.11-2.86 (m, 4H), 2.79-2.67 (m, 1H), 1.34 (d, J = 6.0 Hz, 3H), 1.22 (d, J = 9.1 Hz, 1H), 1.15 (d, J = 6.6 Hz, 3H). |
| 106 107 | 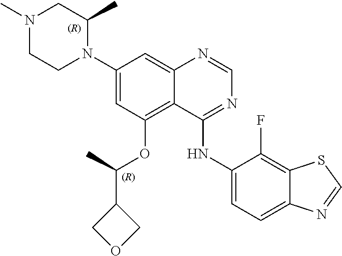 or 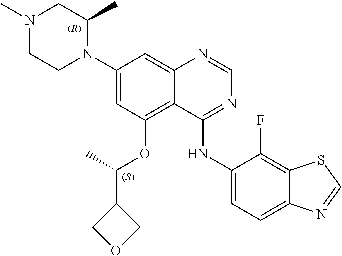 | N-(7-((R)-2,4-dimethylpiperazin-1-yl)-5-((R)-1-(oxetan-3- yl)ethoxy)quinazolin-4-yl)-7-fluorobenzo[d]thiazol-6-amine or N- (7-((R)-2,4-dimethylpiperazin-1-yl)-5-((S)-1-(oxetan-3- yl)ethoxy)quinazolin-4-yl)-7-fluorobenzo[d]thiazol-6-amine First Eluting Isomer: LC-MS: (ES, m/z): RT = 0.891 min, LCMS: m/z = 509 [M + 1], HPLC: RT = 1.509 min; ¹H NMR (400 MHz, DMSO-d6) δ 9.88 (dd, J = 6.0, 1.6 Hz, 1H), 9.41 (d, J = 1.5 Hz, 1H), 8.49 (td, J = 8.2, 6.4 Hz, 1H), 8.34 (s, 1H), 7.98 (d, J = 8.8 Hz, 1H), 6.91 (dd, J = 4.4, 2.3 Hz, 1H), 6.62 (d, J = 2.1 Hz, 1H), 5.36 (p, J = 5.7 Hz, 1H), 4.79 (dtd, J = 7.7, 6.0, 2.0 Hz, 2H), 4.56 (td, J = 6.0, 2.3 Hz, 1H), 4.48 (td, J = 6.0, 2.9 Hz, 1H), 3.46-3.39 (s, 1H), 4.38 (s, 1H), 3.64 (d, J = 12.0 Hz, 1H), 3.17-3.05 (m, 1H), 2.91 (d, J = 11.0 Hz, 1H), 2.78 (d, J = 10.9 Hz, 1H), 2.24 (s, 4H), 2.05 (t, J = 11.2 Hz, 1H), 1.34 (d, J = 5.9 Hz, 3H), 1.16 (d, J = 6.5 Hz, 3H). Second Eluting Isomer: LC-MS: (ES, m/z): RT = 0.893 min, LCMS: m/z = 509 [M + 1], HPLC: RT = 1.926 min ¹H NMR (400 MHz, DMSO-d6) δ 9.88 (dd, J = 6.0, 1.6 Hz, 1H), 9.41 (d, J = 1.5 Hz, 1H), 8.49 (td, J = 8.2, 6.4 Hz, 1H), 8.34 (s, 1H), 7.98 (d, J = 8.8 Hz, 1H), 6.91 (dd, J = 4.4, 2.3 Hz, 1H), 6.62 (d, J = 2.1 Hz, 1H), 5.36 (p, J = 5.7 Hz, 1H), 4.79 (dtd, J = 7.7, 6.0, 2.0 Hz, 2H), 4.56 (td, J = 6.0, 2.3 Hz, 1H), 4.48 (td, J = 6.0, 2.9 Hz, 1H), 4.38 (s, 1H), 3.46-3.39 (s,1H), 3.64 (d, J = 12.0 Hz, 1H), 3.17-3.05 (m, 1H), 2.91 (d, J = 11.0 Hz, 1H), 2.78 (d, J = 10.9 Hz, 1H), 2.24 (s, 4H), 2.05 (t, J = 11.2 Hz, 1H), 1.34 (d, J = 5.9 Hz, 3H), 1.16 (d, J = 6.5 Hz, 3H). |
| 108 109 | 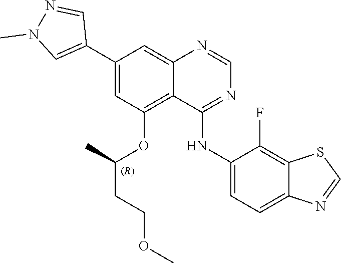 or 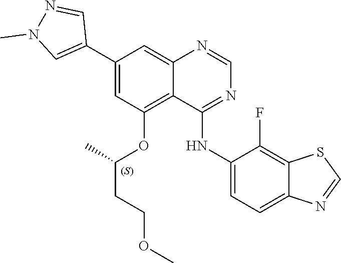 | (S)-7-fluoro-N-(5-((4-methoxybutan-2-yl)oxy)-7-(1-methyl-1H- pyrazol-4-yl)quinazolin-4-yl)benzo[d]thiazol-6-amine OR (R)-7- fluoro-N-(5-((4-methoxybutan-2-yl)oxy)-7-(1-methyl-1H-pyrazol-4- yl)quinazolin-4-yl)benzo[d]thiazol-6-amine First Eluting Isomer: LC-MS: (ES, m/z): RT = 0.958 min, LCMS: m/z = 479 [M + 1], HPLC: RT = 3.016 min; ¹H NMR (300 MHz, DMSO-d6) δ 10.30 (s, 1H), 9.42 (d, J = 1.6 Hz, 1H), 8.71 (dd, J = 8.9, 7.9 Hz, 1H), 8.50 (d, J = 17.1 Hz, 2H), 8.14 (d, J = 0.8 Hz, 1H), 8.03 (d, J = 8.9 Hz, 1H), 7.58 (d, J = 1.4 Hz, 1H), 7.41 (s, 1H), 5.19 (q, J = 6.1 Hz, 1H), 3.91 (s, 3H), 3.45-3.65 (m, 2H), 3.16 (s, 3H), 2.25-2.06 (m, 2H), 1.51 (d, J = 6.0 Hz, 3H). Second Eluting Isomer: LC-MS: (ES, m/z): RT = 0.955 min, LCMS: m/z = 479 [M + 1], HPLC: RT = 3.558 min; ¹H NMR (300 MHz, DMSO-d6) δ 10.29 (d, J = 1.8 Hz, 1H), 9.41 (d, J = 1.5 Hz, 1H), 8.76-8.64 (m, 1H), 8.49 (d, J = 17.7 Hz, 2H), 8.14 (d, J = 0.8 Hz, 1H), 8.02 (d, J = 8.9 Hz, 1H), 7.57 (d, J = 1.4 Hz, 1H), 7.41 (d, J = 1.4 Hz, 1H), 5.19 (q, J = 6.0 Hz, 1H), 3.91 (s, 3H),3.47-3.65 (m, 2H) 3.15 (s, 3H), 2.25-2.06 (m, 2H), 1.51 (d, J = 6.0 |
| 110 111 | 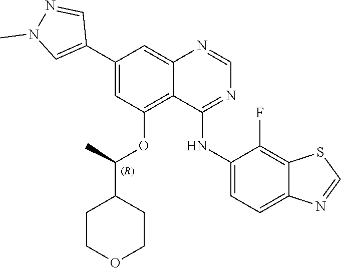 or 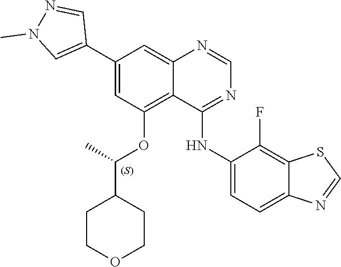 | (R)-7-fluoro-N-(7-(1-methyl-1H-pyrazol-4-yl)-5-(1-(tetrahydro-2H- pyran-4-yl)ethoxy)quinazolin-4-yl)benzo[d]thiazol-6-amine OR (S)-7-fluoro-N-(7-(1-methyl-1H-pyrazol-4-yl)-5-(1-(tetrahydro-2H- pyran-4-yl)ethoxy)quinazolin-4-yl)benzo[d]thiazol-6-amine First Eluting Isomer: LC-MS: (ES, m/z): RT = 1.128 min, LCMS: m/z = 505 [M + 1], HPLC: RT = 2.105 min; ¹H NMR (400 MHz, DMSO-d6) δ 10.27-10.22 (m, 1H), 9.42 (d, J = 1.6 Hz, 1H), 8.77 (dd, J = 8.9, 7.9 Hz, 1H), 8.54 (s, 1H), 8.49 (s, 1H), 8.17 (d, J = 0.8 Hz, 1H), 8.03 (d, J = 8.9 Hz, 1H), 7.58 (d, J = 1.5 Hz, 1H), 7.44 (d, J = 1.6 Hz, 1H), 4.97 (p, J = 6.2 Hz, 1H), 3.92-3.88 (m, 5H) 3.31 (s, 2H), 2.27-2.05 (m, 1H), 1.91 (d, J = 13.0 Hz, 1H), 1.68 (d, J = 12.9 Hz, 1H), 1.52-1.32 (m, 5H). Second Eluting Isomer: LC-MS: (ES, m/z): RT = 1.130 min, LCMS: m/z = 505 [M + 1], HPLC: RT = 3.465 min; ¹H NMR (400 MHz, DMSO-d6) δ 10.25 (d, J = 2.0 Hz, 1H), 9.42 (d, J = 1.5 Hz, 1H), 8.76 (dd, J = 9.0, 7.9 Hz, 1H), 8.54 (s, 1H), 8.48 (s, 1H), 8.17 (d, J = 0.8 Hz, 1H), 8.03 (d, J = 8.9 Hz, 1H), 7.58 (d, J = 1.4 Hz, 1H), 7.44 (d, J = 1.6 Hz, 1H), 4.96 (p, J = 6.3 Hz, 1H), 3.92 (s, 5H), 3.31 (s, 2H), 2.19-2.05 (m, 1H), 1.91 (d, J = 13.1 Hz, 1H), 1.68 (d, J = 12.8 Hz, 1H), 1.49-1.34 (m, 5H). |
| 112 | 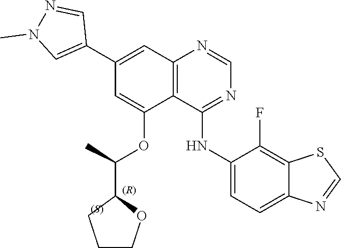 | 7-fluoro-N-(7-(1-methyl-1H-pyrazol-4-yl)-5-((R)-1-((S)- tetrahydrofuran-2-yl)ethoxy)quinazolin-4-yl)benzo[d]thiazol-6- amine LC-MS: (ES, m/z): RT = 1.446 min, LCMS: m/z = 491 [M + 1], ¹H NMR (400 MHz, DMSO-d6) δ 10.16 (s, 1H), 9.44 (d, J = 1.5 Hz, 1H), 8.48 (d, J = 3.6 Hz, 2H), 8.42 (dd, J = 8.8, 7.8 Hz, 1H), 8.16 (d, J = 0.8 Hz, 1H), 8.03 (d, J = 8.8 Hz, 1H), 7.59 (d, J = 1.4 Hz, 1H), 7.51 (d, J = 1.7 Hz, 1H), 5.18-5.07 (m, 1H), 4.24 (td, J = 7.1, 4.4 Hz, 1H), 3.92 (s, 3H), 3.78 (dt, J = 8.2, 6.5 Hz, 1H), 3.68 (dt, J = 8.1, 6.6 Hz, 1H), 2.15-2.03 (m, 1H), 1.92-1.72 (m, 3H), 1.44 (d, J = 6.1 Hz, 3H). |
| 113 | 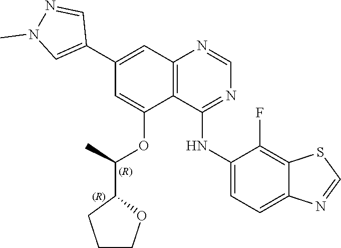 | 7-fluoro-N-(7-(1-methyl-1H-pyrazol-4-yl)-5-((R)-1-((R)- tetrahydrofuran-2-yl)ethoxy)quinazolin-4-yl)benzo[d]thiazol-6- amine LC-MS: (ES, m/z): RT = 1.364 min, LCMS: m/z = 491[M + 1]; ¹H NMR (300 MHz, DMSO-d6) δ 10.26 (s, 1H), 9.45 (d, J = 1.5 Hz, 1H), 8.53-8.45 (m, 2H), 8.35 (t, J = 8.3 Hz, 1H), 8.17 (s, 1H), 8.03 (d, J = 8.8 Hz, 1H), 7.58 (d, J = 1.4 Hz, 1H), 7.46 (s, 1H), 5.02 (q, J = 6.0 Hz, 1H), 4.12 (q, J = 6.8 Hz, 1H), 3.92 (s, 3H), 3.73-3.55 (m, 2H), 2.15-1.99 (m, 1H), 1.92-1.64 (m, 2H), 1.60-1.51 (m, 1H), 1.47 (d, J = 6.1 Hz, 3H). |
| 114 | 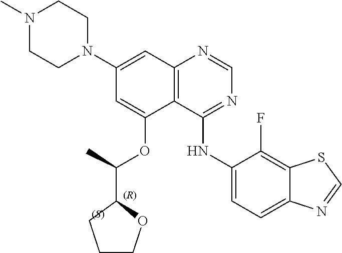 | 7-fluoro-N-(7-(4-methylpiperazin-1-yl)-5-((R)-1-((S)- tetrahydrofuran-2-yl)ethoxy)quinazolin-4-yl)benzo[d]thiazol-6- amine LC-MS: (ES, m/z): RT = 1.428 min, LCMS: m/z = 509 [M + 1], ¹H NMR (400 MHz, DMSO-d6) δ 9.98 (d, J = 1.4 Hz, 1H), 9.41 (d, J = 1.5 Hz, 1H), 8.53-8.42 (m, 1H), 8.35 (s, 1H), 8.00 (d, J = 8.8 Hz, 1H), 6.98 (d, J = 2.3 Hz, 1H), 6.67 (d, J = 2.2 Hz, 1H), 5.07-4.97 (m, 1H), 4.19 (td, J = 7.1, 4.3 Hz, 1H), 3.76 (dt, J = 8.1, 6.4 Hz, 1H), 3.66 (dt, J = 8.1, 6.6 Hz, 1H), 3.40 (d, J = 10.2 Hz, 4H), 3.31-3.28 (m, 4H), 2.26 (s, 3H), 2.12-1.95 (m, 1H), 1.90-1.71 (m, 3H), 1.38 (d, J = 6.1 Hz, 3H). |
| 115 | 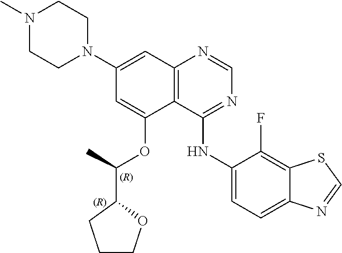 | 7-fluoro-N-(7-(4-methylpiperazin-1-yl)-5-((R)-1-((R)- tetrahydrofuran-2-yl)ethoxy)quinazolin-4-yl)benzo[d]thiazol-6- amine LC-MS: (ES, m/z): RT = 1.329 min, LCMS: m/z = 509 [M + 1]; ¹H NMR (300 MHz, DMSO-d6) δ 10.04 (s, 1H), 9.42 (d, J = 1.6 Hz, 1H), 8.49-8.27 (m, 2H), 8.00 (d, J = 8.8 Hz, 1H), 6.92 (s, 1H), 6.66 (d, J = 2.1 Hz, 1H), 4.93 (t, J = 6.1 Hz, 1H), 4.06 (t, J = 6.6 Hz, 1H), 3.63 (q, J = 6.4 Hz, 2H), 3.51-3.37 (m,4H), 3.32-3.24 (m, 4H), 2.62-2.53 (m, 2H), 2.30 (s, 3H), 2.09-1.96 (m, 1H), 1.85 (t, J = 6.9 Hz, 2H), 1.72 (d, J = 10.9 Hz, 1H), 1.41 (d, J = 6.1 Hz, 3H). |
| 116 117 | 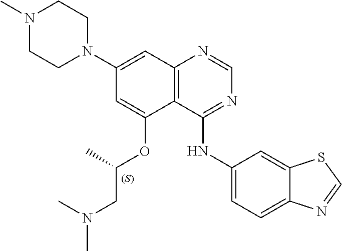 or 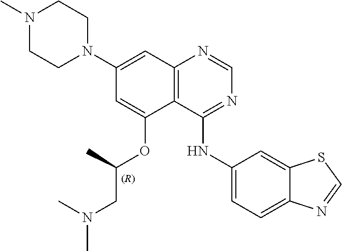 | (R)-N-(5-((1-(dimethylamino)propan-2-yl)oxy)-7-(4- methylpiperazin-1-yl)quinazolin-4-yl)benzo[d]thiazol-6-amine OR (S)-N-(5-((1-(dimethylamino)propan-2-yl)oxy)-7-(4- methylpiperazin-1-yl)quinazolin-4-yl)benzo[d]thiazol-6-amine First Eluting Isomer: LC-MS: (ES, m/z): RT = 1.015 min, LCMS: m/z = 478.15 [M + 1], Chiral-HPLC (ES): RT = 1.426, ¹H NMR (400 MHz, DMSO-d6) δ 10.40 (s, 1H), 9.29 (s, 1H), 8.80 (d, J = 2.1 Hz, 1H), 8.40 (s, 1H), 8.08 (d, J = 8.8 Hz, 1H), 7.76 (dd, J = 8.8, 2.2 Hz, 1H), 6.87 (d, J = 2.3 Hz, 1H), 6.65 (d, J = 2.1 Hz, 1H), 5.03 (s, 1H), 3.42 (s, 4H), 3.00 (s, 1H), 2.57 (s, 4H), 2.42-2.51 (m, 1H), 2.32 (s, 3H), 2.26 (s, 6H), 1.46 (d, J = 5.9 Hz, 3H). Second Eluting Isomer: LC-MS: (ES, m/z): RT = 1.076 min, LCMS: m/z = 478.15 [M + 1], Chiral-HPLC (ES): RT = 1.887, ¹H NMR (400 MHz, DMSO-d6) δ 10.42 (s, 1H), 9.28 (s, 1H), 8.80 (d, J = 2.1 Hz, 1H), 8.39 (s, 1H), 8.07 (d, J = 8.8 Hz, 1H), 7.76 (dd, J = 8.8, 2.2 Hz, 1H), 6.85 (d, J = 2.3 Hz, 1H), 6.63 (d, J = 2.2 Hz, 1H), 5.01 (ddd, J = 9.3, 6.1, 4.0 Hz, 1H), 3.52-3.63 (m, 4H), 2.91-2.98 (m, 1H), 2.51- 2.40 (m, 5H), 2.24 (d, J = 6.5 Hz, 9H), 1.46 (d, J = 5.9 Hz, 3H). |
| 118 119 | 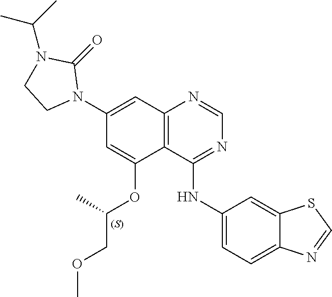 or 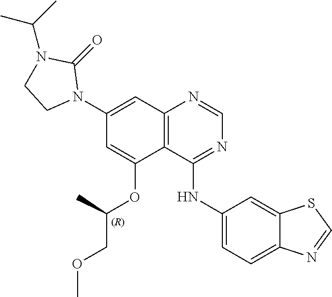 | (R)-1-(4-(benzo[d]thiazol-6-ylamino)-5-((1-methoxypropan-2- yl)oxy)quinazolin-7-yl)-3-isopropylimidazolidin-2-one OR (S)-1- (4-(benzo[d]thiazol-6-ylamino)-5-((1-methoxypropan-2- yl)oxy)quinazolin-7-yl)-3-isopropylimidazolidin-2-one First Eluting Isomer: LC-MS: (ES, m/z): RT = 1.188 min, LCMS: m/z = 493.10 [M + 1], Chiral-HPLC (ES): RT = 2.408,¹H NMR (400 MHz, DMSO-d6) 8 10.41 (s, 1H), 9.30 (s, 1H), 8.92 (d, J = 2.1 Hz, 1H), 8.52 (s, 1H), 8.15-8.07 (m, 2H), 7.77 (dd, J = 8.9, 2.2 Hz, 1H), 7.06 (d, J = 2.0 Hz, 1H), 4.96 (s, 1H), 4.10 (p, J = 6.7 Hz, 1H), 3.94 (t, J = 8.3 Hz, 2H), 3.84 (dd, J = 10.8, 2.9 Hz, 1H), 3.77 (d, J = 4.5 Hz, 1H), 3.52-3.54 (m, 4H), 1.50 (d, J = 6.3 Hz, 3H), 1.16 (dd, J = 6.8, 1.7 Hz, 6H). Second Eluting Isomer: LC-MS: (ES, m/z): RT = 1.188 min, LCMS: m/z = 493.10 [M + 1], Chiral-HPLC (ES): RT = 2.408, ¹H NMR (400 MHz, DMSO-d6) δ 10.41 (s, 1H), 9.30 (s, 1H), 8.92 (d, J = 2.1 Hz, 1H), 8.52 (s, 1H), 8.15-8.07 (m, 2H), 7.77 (dd, J = 8.9, 2.2 Hz, 1H), 7.06 (d, J = 2.0 Hz, 1H), 4.96 (s, 1H), 4.10 (p, J = 6.7 Hz, 1H), 3.94 (t, J = 8.3 Hz, 2H), 3.84 (dd, J = 10.8, 2.9 Hz, 1H), 3.77 (d, J = 4.5 Hz, 1H), 3.52-3.54 (m, 4H), 1.50 (d, J = 6.3 Hz, 3H), 1.16 (dd, J = 6.8, 1.7 Hz, 6H). |
| 120 121 | 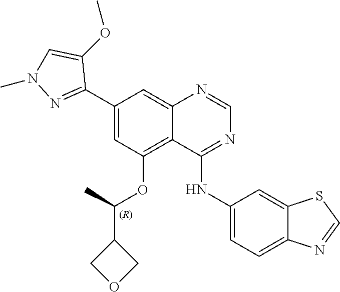 or 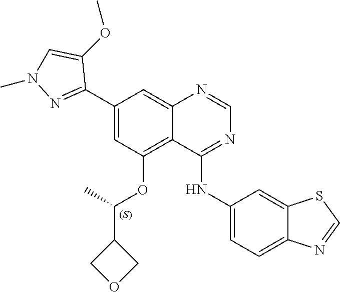 | (S)-N-(7-(4-methoxy-1-methyl-1H-pyrazol-3-yl)-5-(1-(oxetan-3- yl)ethoxy)quinazolin-4-yl)benzo[d]thiazol-6-amine OR (R)-N-(7- (4-methoxy-1-methyl-1H-pyrazol-3-yl)-5-(1-(oxetan-3- yl)ethoxy)quinazolin-4-yl)benzo[d]thiazol-6-amine First Eluting Isomer: LC-MS: (ES, m/z): RT = 1.059 min, LCMS: m/z = 489 [M + 1], Chiral-HPLC: tR = 3.184 min, ¹H NMR (300 MHz, DMSO-d6) δ 10.14 (s, 1H), 9.31 (s, 1H), 8.71 (d, J = 2.1 Hz, 1H), 8.52 (s, 1H), 8.07 (d, J = 8.8 Hz, 1H), 7.87 (d, J = 1.3 Hz, 1H), 7.76 (dd, J = 8.8, 2.1 Hz, 1H), 7.70 (s, 1H), 7.66 (d, J = 1.5 Hz, 1H), 5.15 (q, J = 6.0 Hz, 1H), 4.81 (ddd, J = 10.3, 7.9, 6.4 Hz, 2H), 4.61 (dt, J = 18.9, 6.0 Hz, 2H), 3.85 (d, J = 7.8 Hz, 6H), 1.42 Second Eluting Isomer: LC-MS: (ES, m/z): RT = 1.058 min, LCMS: m/z = 489 [M + 1], Chiral-HPLC: tR = 4.007 min, ¹H NMR (300 MHz, DMSO-d6) δ 10.14 (s, 1H), 9.31 (s, 1H), 8.71 (d, J = 2.1 Hz, 1H), 8.52 (s, 1H), 8.07 (d, J = 8.8 Hz, 1H), 7.87 (d, J = 1.3 Hz, 1H), 7.76 (dd, J = 8.8, 2.1 Hz, 1H), 7.70 (s, 1H), 7.66 (d, J = 1.5 Hz, 1H), 5.15 (q, J = 6.0 Hz, 1H), 4.81 (ddd, J = 10.3, 7.9, 6.4 Hz, 2H), 4.61 (dt, J = 18.9, 6.0 Hz, 2H), 3.85 (d, J = 7.8 Hz, 6H), 1.42 (d, J = 6.0 Hz, 3H). |
| 122 123 | 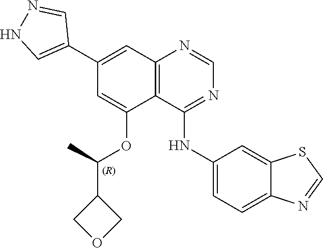 or 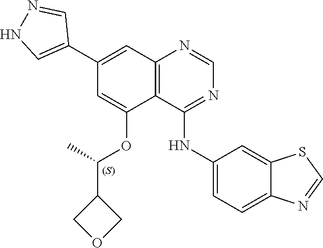 | (R)-N-(5-(1-(oxetan-3-yl)ethoxy)-7-(1H-pyrazol-4-yl)quinazolin-4- yl)benzo[d]thiazol-6-amine OR (S)-N-(5-(1-(oxetan-3-yl)ethoxy)- 7-(1H-pyrazol-4-yl)quinazolin-4-yl)benzo[d]thiazol-6-amine First Eluting Isomer: LC-MS: (ES, m/z): RT = 0.924 min, LCMS: m/z = 445 [M + 1], Chiral-HPLC: RT = 2.030 min, ¹H NMR (300 MHz, DMSO-d6) δ 10.20 (s, 1H), 9.33 (s, 1H), 8.76 (d, J = 2.2 Hz, 1H), 8.55 (s, 3H), 8.10 (d, J = 8.8 Hz, 1H), 7.79 (dd, J = 8.9, 2.2 Hz, 1H), 7.64 (d, J = 1.4 Hz, 1H), 7.53 (s, 1H), 5.43 (p, J = 6.0 Hz, 1H), 4.85 (dt, J = 8.1, 6.2 Hz, 2H), 4.65 (t, J = 6.1 Hz, 1H), 4.57 (t, J = 6.0 Hz, 1H), 3.60-3.49 (m, 1H), 1.42 (d, J = 6.0 Hz, 3H), 1.24 (s, 1H). Second Eluting Isomer: LC-MS: (ES, m/z): RT = 0.930 min, LCMS: m/z = 445 [M + 1], Chiral-HPLC: RT = 2.537 min, ¹H NMR (300 MHz, DMSO-d6) δ 10.20 (s, 1H), 9.33 (s, 1H), 8.76 (d, J = 2.2 Hz, 1H), 8.55 (s, 3H), 8.10 (d, J = 8.8 Hz, 1H), 7.79 (dd, J = 8.9, 2.2 Hz, 1H), 7.64 (d, J = 1.4 Hz, 1H), 7.53 (s, 1H), 5.43 (p, J = 6.0 Hz, 1H), 4.85 (dt, J = 8.1, 6.2 Hz, 2H), 4.65 (t, J = 6.1 Hz, 1H), 4.57 (t, J = 6.0 Hz, 1H), 3.60-3.49 (m, 1H), 1.42 (d, J = 6.0 Hz, 3H), 1.24 (s, 1H). |
| 124 | 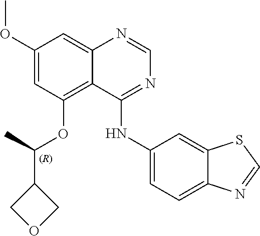 | (R)-N-(7-methoxy-5-(1-(oxetan-3-yl)ethoxy)quinazolin-4- yl)benzo[d]thiazol-6-amine LC-MS: (ES, m/z): RT = 0.851 min, LCMS: m/z = 409 [M + 1]; ¹H NMR (400 MHz, DMSO-d6) δ 10.00 (s, 1H), 9.31 (s, 1H), 8.70 (d, J = 2.1 Hz, 1H), 8.48 (s, 1H), 8.07 (d, J = 8.8 Hz, 1H), 7.75 (dd, J = 8.8, 2.2 Hz, 1H), 6.89-6.82 (m, 2H), 5.19 (p, J = 6.0 Hz, 1H), 4.81 (ddd, J = 12.1, 8.0, 6.3 Hz, 2H), 4.62 (t, J = 6.1 Hz, 1H), 4.53 (t, J = 5.9 Hz, 1H), 3.92 (s, 3H), 3.49-3.39 (s, 1H), 1.37 (d, J = 6.0 Hz, 3H). |
| 125 | 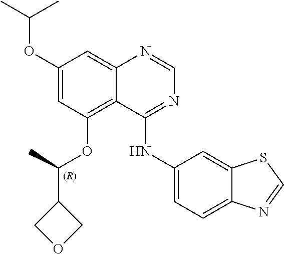 | (R)-N-(7-isopropoxy-5-(1-(oxetan-3-yl)ethoxy)quinazolin-4- yl)benzo[d]thiazol-6-amine LC-MS: (ES, m/z): RT = 0.866 min, LCMS: m/z = 437 [M + 1];¹H NMR (400 MHz, DMSO-d6) δ 10.00 (s, 1H), 9.31 (s, 1H), 8.71 (d, J = 2.1 Hz, 1H), 8.47 (s, 1H), 8.07 (d, J = 8.8 Hz, 1H), 7.75 (dd, J = 8.8, 2.2 Hz, 1H), 6.82 (q, J = 2.3 Hz, 2H), 5.20 (p, J = 6.0 Hz, 1H), 4.93-4.75 (m, 3H), 4.61 (t, J = 6.1 Hz, 1H), 4.53 (t, J = 6.0 Hz, 1H), 3.48 (dd, J = 8.0, 5.8 Hz, 1H), 1.40-1.32 (m, 9H). |
| 126 | 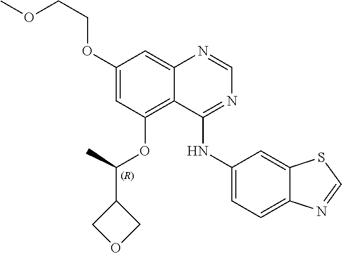 | (R)-N-(7-(2-methoxyethoxy)-5-(1-(oxetan-3-yl)ethoxy)quinazolin- 4-yl)benzo[d]thiazol-6-amine LC-MS: (ES, m/z): RT = 0.844 min, LCMS: m/z = 453 [M + 1]; ¹H NMR (400 MHz, DMSO-d6) δ 10.01 (s, 1H), 9.31 (s, 1H), 8.70 (d, J = 2.1 Hz, 1H), 8.48 (s, 1H), 8.07 (d, J = 8.7 Hz, 1H), 7.75 (dd, J = 8.8, 2.2 Hz, 1H), 6.89 (d, J = 2.4 Hz, 1H), 6.84 (d, J = 2.3 Hz, 1H), 5.22 (p, J = 6.0 Hz, 1H), 4.81 (ddd, J = 12.1, 8.0, 6.3 Hz, 2H), 4.61 (t, J = 6.1 Hz, 1H), 4.52 (t, J = 5.9 Hz, 1H), 4.31-4.24 (m, 2H), 3.77- 3.70 (m, 2H), 3.49-3.42 (m, 1H), 3.34 (s, 3H), 1.37 (d, J = 6.0 Hz, 3H). |
| 127 128 | 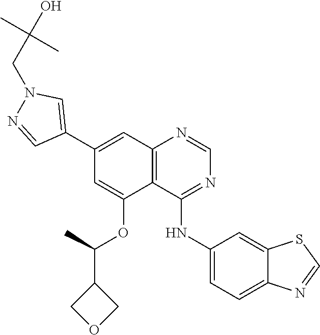 [SMILES: C[C@@H](Oc1cc(-c2cnn(CC(C)(C)O)c2)cc2ncnc(Nc3ccc4ncsc4c3)c12)C1COC1] or 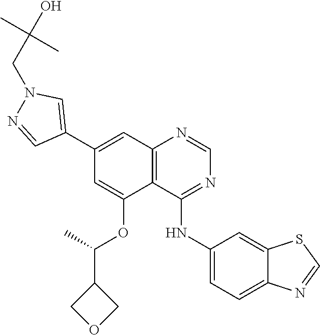 [SMILES: C[C@H](Oc1cc(-c2cnn(CC(C)(C)O)c2)cc2ncnc(Nc3ccc4ncsc4c3)c12)C1COC1] | (R)-1-(4-(4-(benzo[d]thiazol-6-ylamino)-5-(1-(oxetan-3- yl)ethoxy)quinazolin-7-yl)-1H-pyrazol-1-yl)-2-methylpropan-2-ol, OR (S)-1-(4-(4-(benzo[d]thiazol-6-ylamino)-5-(1-(oxetan-3- yl)ethoxy)quinazolin-7-yl)-1H-pyrazol-1-yl)-2-methylpropan-2-ol First Eluting Isomer: LC-MS: (ES, m/z): RT = 0.803 min, LCMS: m/z = 517 [M + 1], HPLC: RT = 3.370 min; ¹H NMR (400 MHz, DMSO-d6) δ 10.18 (s, 1H), 9.33 (d, J = 2.5 Hz, 1H), 8.76 (d, J = 2.2 Hz, 1H), 8.54 (d, J = 1.5 Hz, 1H), 8.46 (s, 1H), 8.20 (s, 1H), 8.10 (d, J = 8.8 Hz, 1H), 7.80 (dd, J = 8.8, 2.2 Hz, 1H), 7.60 (d, J = 1.5 Hz, 1H), 7.50 (s, 1H), 5.45 (q, J = 6.2 Hz, 1H), 4.90-4.79 (m, 3H), 4.66 (t, J = 6.1 Hz, 1H), 4.57 (t, J = 6.0 Hz, 1H), 4.09 (s, 2H), 3.53 (q, J = 6.8 Hz, 1H), 1.42 (d, J = 6.0 Hz, 3H), 1.14 (d, J = 2.7 Hz, 6H). Second Eluting Isomer: LC-MS: (ES, m/z): RT = 0.803 min, LCMS: m/z = 517 [M + 1], HPLC: RT = 4.200 min; ¹H NMR (400 MHz, DMSO-d6) δ 10.16 (s, 1H), 9.33 (s, 1H), 8.77 (d, J = 2.1 Hz, 1H), 8.53 (s, 1H), 8.45 (s, 1H), 8.20 (s, 1H), 8.09 (d, J = 8.8 Hz, 1H), 7.80 (dd, J = 8.9, 2.2 Hz, 1H), 7.60 (d, J = 1.4 Hz, 1H), 7.50 (d, J = 1.6 Hz, 1H), 5.44 (p, J = 6.0 Hz, 1H), 4.90-4.79 (m, 3H), 4.65 (t, J = 6.1 Hz, 1H), 4.57 (t, J = 6.0 Hz, 1H), 4.08 (s, 2H), 3.54 (q, J = 6.8, 6.4 Hz, 1H), 1.42 (d, J = 6.0 Hz, 3H), 1.14 (d, J = 2.7 Hz, 6H). |
| 129 | 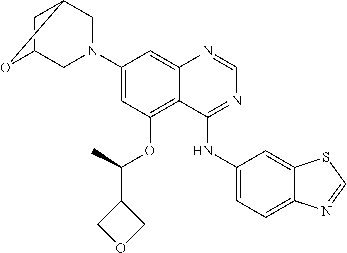 [SMILES: C[C@@H](Oc1cc(N2CC3CC(C2)O3)cc2ncnc(Nc3ccc4ncsc4c3)c12)C1COC1] | N-(7-(6-oxa-3-azabicyclo[3.1.1]heptan-3-yl)-5-((R)-1-(oxetan-3- yl)ethoxy)quinazolin-4-yl)benzo[d]thiazol-6-amine LC-MS: (ES, m/z): RT = 0.857 min, LCMS: m/z = 476 [M + 1], ¹H NMR (300 MHz, DMSO-d6) δ 9.83 (s, 1H), 9.17 (s, 1H), 8.65 (d, J = 2.1 Hz, 1H), 8.26 (s, 1H), 7.94 (d, J = 8.8 Hz, 1H), 7.64 (dd, J = 8.7, 2.1 Hz, 1H), 6.60 (d, J = 2.3 Hz, 1H), 6.42 (d, J = 2.1 Hz, 1H), 5.20 (q, J = 6.3 Hz, 1H), 4.69 (dt, J = 13.1, 6.6 Hz, 4H), 4.50 (t, J = 6.1 Hz, 1H), 4.42 (t, J = 6.0 Hz, 1H), 3.69-3.55 (m, 2H), 3.45 (ddd, J = 29.1, 12.7, 6.8 Hz, 3H), 3.06 (q, J = 7.4 Hz, 1H), 1.82 (d, J = 8.8 Hz, 1H), 1.29 (d, J = 5.9 Hz, 3H). |
| 130 | 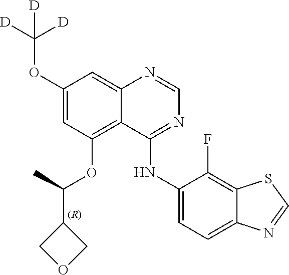 | (R)-7-fluoro-N-(7-(methoxy-d3)-5-(1-(oxetan-3- yl)ethoxy)quinazolin-4-yl)benzo[d]thiazol-6-amine LC-MS: (ES, m/z): RT-0.880 min, LCMS: m/z = 430 [M + 1], ¹H NMR (400 MHz, DMSO-d6) δ 9.91 (s, 1H), 9.41 (d, J = 1.5 Hz, 1H), 8.42 (d, J = 3.1 Hz, 1H), 8.31 (dt, J = 8.9, 7.1 Hz, 1H), 7.97 (d, J = 8.8 Hz, 1H), 6.85 (dd, J = 16.0, 2.3 Hz, 2H), 5.19 (q, J = 6.1 Hz, 1H), 4.77 (ddd, J = 7.8, 6.3, 3.5 Hz, 2H), 4.54 (t, J = 5.9 Hz, 1H), 4.45 (t, J = 5.9 Hz, 1H), 3.36 (d, J = 6.3 Hz, 1H), 1.34 (d, J = 6.0 Hz, 3H). |
| 131 | 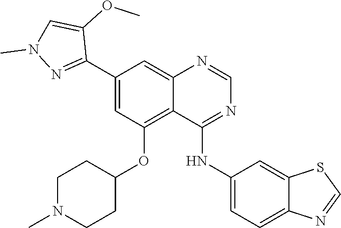 [SMILES: COc1cn(C)nc1-c1cc(OC2CCN(C)CC2)c2c(Nc3ccc4ncsc4c3)ncnc2c1] | N-(7-(4-methoxy-1-methyl-1H-pyrazol-3-yl)-5-((1-methylpiperidin- 4-yl)oxy)quinazolin-4-yl)benzo[d]thiazol-6-amine LC-MS: (ES, m/z): RT = 1.057 min, LCMS: m/z = 502 [M + 1], 1H NMR (400 MHz, DMSO-d6) δ 10.28 (s, 1H), 9.32 (s, 1H), 8.94- 8.84 (m, 1H), 8.57 (s, 1H), 8.12 (d, J = 8.8 Hz, 1H), 7.87 (d, J = 1.3 Hz, 1H), 7.76 (dd, J = 8.8, 2.2 Hz, 1H), 7.71 (s, 1H), 7.65 (d, J = 1.5 Hz, 1H), 4.83 (s, 1H), 3.86 (d, J = 10.7 Hz, 6H), 2.75 (d, J = 11.5 Hz, 2H), 2.46-2.32 (m, 2H), 2.24 (s, 5H), 2.02 (d, J = 10.6 Hz, 2H). |
| 132 | 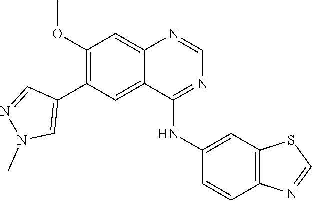 [SMILES: COc1cc2ncnc(Nc3ccc4ncsc4c3)c2cc1-c1cnn(C)c1] | N-(7-methoxy-6-(1-methyl-1H-pyrazol-4-yl)quinazolin-4- yl)benzo[d]thiazol-6-amine LC-MS: (ES, m/z): RT = 2.081 min, LCMS: m/z = 389 [M + 1], ¹H- NMR 1H NMR (400 MHz, DMSO-d6) δ 9.31 (s, 1H), 8.80-8.72 (m, 2H), 8.55 (d, J = 2.1 Hz, 1H), 8.25 (s, 1H), 8.16-8.08 (m, 2H), 7.92 (ddd, J = 8.8, 4.2, 2.2 Hz, 1H), 7.28 (s, 1H), 4.05 (s, 3H), 3.93 (s, 3H). |
| 133 | 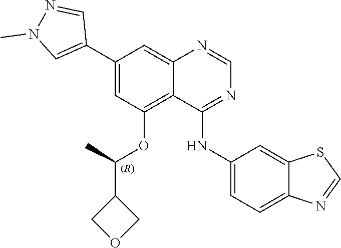 | (R)-N-(7-(1-methyl-1H-pyrazol-4-yl)-5-(1-(oxetan-3- yl)ethoxy)quinazolin-4-yl)benzo[d]thiazol-6-amine LC-MS: (ES, m/z): RT = 0.820 min, LCMS: m/z = 459 [M + 1]; ¹H NMR (400 MHz, DMSO-d6) δ 10.17 (s, 1H), 9.33 (s, 1H), 8.76 (d, J = 2.2 Hz, 1H), 8.51 (d, J = 17.0 Hz, 2H), 8.18 (d, J = 0.8 Hz, 1H), 8.09 (d, J = 8.8 Hz, 1H), 7.79 (dd, J = 8.9, 2.1 Hz, 1H), 7.58 (d, J = 1.4 Hz, 1H), 7.48 (d, J = 1.6 Hz, 1H), 5.41 (p, J = 6.1 Hz, 1H), 4.90- 4.80 (m, 2H), 4.65 (t, J = 6.1 Hz, 1H), 4.56 (t, J = 6.0 Hz, 1H), 3.92 (s, 3H), 3.54 (qd, J = 7.9, 6.9, 2.0 Hz, 1H), 1.42 (d, J = 6.0 Hz, 3H). |
| 134 | 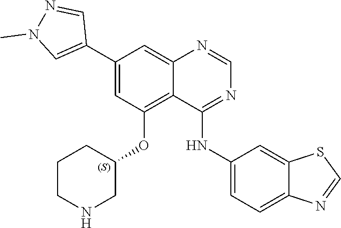 | (S)-N-(7-(1-methyl-1H-pyrazol-4-yl)-5-(piperidin-3- yloxy)quinazolin-4-yl)benzo[d]thiazol-6-amine LC-MS: (ES, m/z): RT = 0.966 min, LCMS: m/z = 458 [M + 1], ¹H NMR (300 MHz, DMSO-d6) δ 10.97 (s, 1H), 9.31 (s, 1H), 8.94 (s, 1H), 8.54 (s, 1H), 8.46 (s, 1H), 8.15 (s, 1H), 8.07 (d, J = 1.3 Hz, 2H), 7.55 (d, J = 1.4 Hz, 1H), 7.42 (s, 1H), 5.16 (s, 1H), 3.91 (s, 3H), 3.20 (s, 1H), 2.95-3.02 (m, 2H), 2.93 (s, 1H), 2.07 (s, 1H), 1.79 (m , 2H), 1.50 (s, 1H). |
| 135 | 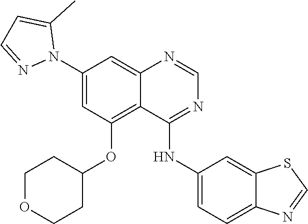 [SMILES: Cc1ccnn1-c1cc(OC2CCOCC2)c2c(Nc3ccc4ncsc4c3)ncnc2c1] | N-(7-(5-methyl-1H-pyrazol-1-yl)-5-((tetrahydro-2H-pyran-4- yl)oxy)quinazolin-4-yl)benzo[d]thiazol-6-amine LC-MS: (ES, m/z): RT = 1.077 min, m/z = 459 [M + 1], ¹H NMR (400 MHz, DMSO-d6) δ 10.30 (s, 1H), 9.34 (s, 1H), 8.91 (d, J = 2.1 Hz, 1H), 8.64 (s, 1H), 8.13 (d, J = 8.8 Hz, 1H), 7.81 (dd, J = 8.8, 2.2 Hz, 1H), 7.70 (d, J = 1.7 Hz, 1H), 7.51-7.45 (m, 2H), 6.39 (t, J = 1.3 Hz, 1H), 5.13 (tt, J = 9.0, 4.2 Hz, 1H), 3.95 (dt, J = 11.9, 4.2 Hz, 2H), 3.59 (ddd, J = 11.9, 10.1, 2.5 Hz, 2H), 2.54-2.48 (m, 3H), 2.30- 2.22 (m, 2H), 2.04-1.90 (m, 2H). |
| 136 | 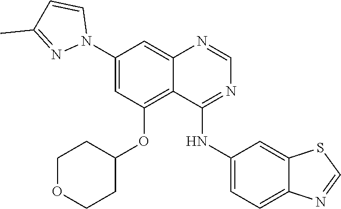 [SMILES: Cc1ccn(-c2cc(OC3CCOCC3)c3c(Nc4ccc5ncsc5c4)ncnc3c2)n1] | N-(7-(3-methyl-1H-pyrazol-1-yl)-5-((tetrahydro-2H-pyran-4- yl)oxy)quinazolin-4-yl)benzo[d]thiazol-6-amine LC-MS: (ES, m/z): RT = 1.166 min, m/z = 459 [M + 1], ¹H NMR (400 MHz, DMSO-d6) δ 10.23 (s, 1H), 9.33 (s, 1H), 8.89 (d, J = 2.1 Hz, 1H), 8.72 (d, J = 2.5 Hz, 1H), 8.59 (s, 1H), 8.12 (d, J = 8.7 Hz, 1H), 7.82-7.73 (m, 2H), 7.66 (d, J = 2.2 Hz, 1H), 6.46 (d, J = 2.5 Hz, 1H), 5.18 (tt, J = 9.0, 4.3 Hz, 1H), 3.97 (dt, J = 11.8, 4.2 Hz, 2H), 3.62 (ddd, J = 11.9, 10.0, 2.6 Hz, 2H), 2.33 (s, 3H), 2.33-2.23 (m, 2H), 1.96 (dtd, J = 13.5, 9.6, 4.2 Hz, 2H). |
| 139 | 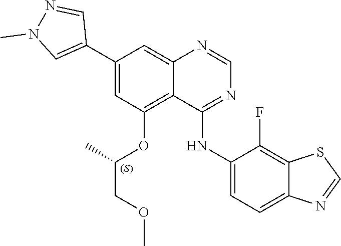 | (S)-7-fluoro-N-(5-((1-methoxypropan-2-yl)oxy)-7-(1-methyl-1H- pyrazol-4-yl)quinazolin-4-yl)benzo[d]thiazol-6-amine LC-MS: (ES, m/z): RT = 1.078 min, LCMS: m/z = 465 [M + 1]¹H NMR (400 MHz, DMSO-d6) δ 10.25 (s, 1H), 9.44 (d, J = 1.6 Hz, 1H), 8.55-8.46 (m, 3H), 8.16 (d, J = 0.8 Hz, 1H), 8.03 (d, J = 8.9 Hz, 1H), 7.59 (d, J = 1.4 Hz, 1H), 7.47 (d, J = 1.6 Hz, 1H), 5.30- 5.21 (m, 1H), 3.92 (s, 3H), 3.79-3.67 (m, 2H), 3.28 (s, 3H), 1.47 (d, J = 6.2 Hz, 3H). |
| 140 | 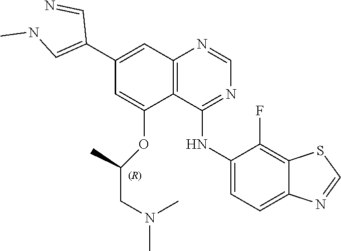 | (R)-N-(5-((1-(dimethylamino)propan-2-yl)oxy)-7-(1-methyl-1H- pyrazol-4-yl)quinazolin-4-yl)-7-fluorobenzo[d]thiazol-6-amine LC-MS: (ES, m/z): RT = 0.698 min, LCMS: m/z = 478 [M + 1], ¹HNMR 1H NMR (300 MHz, DMSO-d6) δ 10.57 (s, 1H), 8.92 (dd, J = 4.2, 1.6 Hz, 1H), 8.71 (dt, J = 13.1, 8.8 Hz, 1H), 8.54-8.43 (m, 3H), 8.14 (s, 1H), 7.93 (d, J = 9.3 Hz, 1H), 7.67-7.53 (m, 2H), 7.41 (d, J = 1.6 Hz, 1H), 5.11 (s, 1H), 3.90 (s, 3H), 3.32(m,1H)2.95-2.82 (m, 1H), 2.17 (s, 6H), 1.50 (d, J = 5.9 Hz, 3H). |
| 141 | 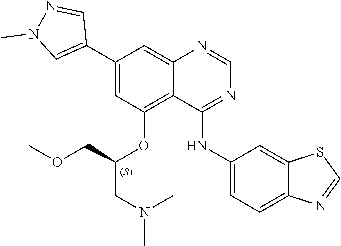 | (S)-N-(5-((1-(dimethylamino)-3-methoxypropan-2-yl)oxy)-7-(1- methyl-1H-pyrazol-4-yl)quinazolin-4-yl)benzo[d]thiazol-6-amine LC-MS: (ES, m/z): RT = 1.101 min, LCMS: m/z = 490 [M + 1]. ¹H NMR (400 MHz, DMSO-d6) δ 10.57 (s, 1H), 9.32 (s, 1H), 8.92 (dd, J = 5.8, 2.2 Hz, 1H), 8.56 (d, J = 2.2 Hz, 1H), 8.41 (s, 1H), 8.16- 8.08 (m, 2H), 7.76 (dd, J = 8.8, 2.1 Hz, 1H), 7.60-7.53 (m, 2H), 5.21 (s, 1H), 3.92 (s, 3H), 3.83 (m, 2H), 3.36 (s, 3H), 2.89 (s, 1H), 2.77-2.63 (m, 1H), 2.27 (s, 6H) |
| 142 143 | 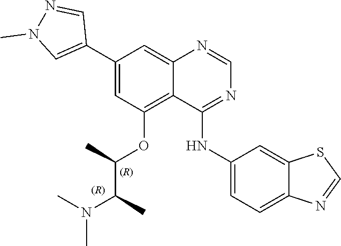 or 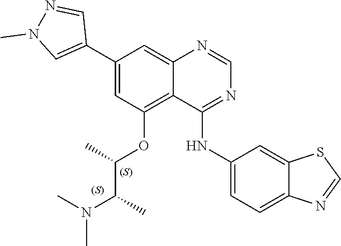 | N-(5-(((2S,3S)-3-(dimethylamino)butan-2-yl)oxy)-7-(1-methyl-1H- pyrazol-4-yl)quinazolin-4-yl)benzo[d]thiazol-6-amine OR N-(5- (((2R,3R)-3-(dimethylamino)butan-2-yl)oxy)-7-(1-methyl-1H- pyrazol-4-yl)quinazolin-4-yl)benzo[d]thiazol-6-amine First Eluting Isomer: LC-MS: (ES, m/z): RT = 0.929 min, m/z = 474 [M + 1], chiral-HPLC: R = 2.136 min, ¹H NMR (300 MHz, DMSO-d6) δ 10.89 (s, 1H), 9.33 (s, 1H), 8.72 (d, J = 2.1 Hz, 1H), 8.48 (d, J = 6.4 Hz, 2H), 8.18-8.06 (m, 2H), 7.82-7.73 (m, 1H), 7.53 (s, 1H), 7.34 (s, 1H), 4.83 (s, 1H), 3.92 (s, 3H), 3.17 (s, 1H), 2.15 (s, 6H), 1.51 (d, J = 5.7 Hz, 3H), 1.04 (s, 3H). Second Eluting Isomer: LC-MS: (ES, m/z): RT = 1.134 min, m/z = 474 [M + 1], chiral-HPLC: R = 2.875 min, ¹H NMR (300 MHz, DMSO-d6) δ 10.90 (s, 1H), 9.33 (s, 1H), 8.72 (s, 1H), 8.48 (d, J = 6.1 Hz, 2H), 8.18-8.06 (m, 2H), 7.77 (d, J = 8.9 Hz, 1H), 7.53 (s, 1H), 7.33 (s, 1H), 4.82 (s, 1H), 3.92 (s, 3H), 3.16 (s, 1H), 2.14 (s, 6H), 1.51 (d, J = 5.8 Hz, 3H), 1.03 (s, 3H). |
| 144 145 | 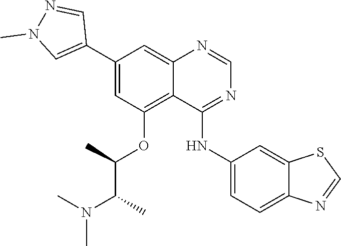 [SMILES: C[C@@H](Oc1cc(-c2cnn(C)c2)cc2ncnc(Nc3ccc4ncsc4c3)c12)[C@H](C)N(C)C] or 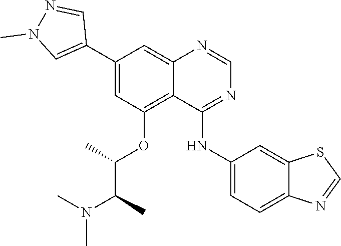 [SMILES: C[C@H](Oc1cc(-c2cnn(C)c2)cc2ncnc(Nc3ccc4ncsc4c3)c12)[C@@H](C)N(C)C] | N-(5-(((2S,3R)-3-(dimethylamino)butan-2-yl)oxy)-7-(1-methyl-1H- pyrazol-4-yl)quinazolin-4-yl)benzo[d]thiazol-6-amine OR N-(5- (((2R,3S)-3-(dimethylamino)butan-2-yl)oxy)-7-(1-methyl-1H- pyrazol-4-yl)quinazolin-4-yl)benzo[d]thiazol-6-amine First Eluting Isomer: LC-MS: (ES, m/z): RT = 0.792 min, LCMS: m/z = 474.25 [M + 1], Chiral-HPLC (ES): RT = 1.838, ¹H NMR (400 MHz, DMSO-d6) δ 10.48 (s, 1H), 9.33 (s, 1H), 8.83 (d, J = 2.1 Hz, 1H), 8.50 (d, J = 16.9 Hz, 2H), 8.17 (d, J = 0.8 Hz, 1H), 8.12 (d, J = 8.8 Hz, 1H), 7.76 (dd, J = 8.8, 2.2 Hz, 1H), 7.56 (d, J = 1.5 Hz, 1H), 7.44 (d, J = 1.6 Hz, 1H), 5.07 (s, 1H), 3.92 (s, 3H), 2.90 (s, 1H), 2.28 (s, 6H), 1.50 (d, J = 6.1 Hz, 3H), 1.15 (d, J = 5.7 Hz, 3H). Second Eluting Isomer: LC-MS: (ES, m/z): RT = 0.792 min, LCMS: m/z = 474.25 [M + 1], Chiral-HPLC (ES): RT = 2.267, ¹H NMR (400 MHz, DMSO-d6) δ 10.48 (s, 1H), 9.33 (s, 1H), 8.83 (d, J = 2.2 Hz, 1H), 8.51 (d, J = 16.9 Hz, 2H), 8.17 (d, J = 0.8 Hz, 2H), 7.76 (dd, J = 8.8, 2.2 Hz, 1H), 7.56 (d, J = 1.4 Hz, 1H), 7.44 (d, J = 1.6 Hz, 1H), 5.07 (s, 1H), 3.92 (s, 3H), 2.90 (s, 1H), 2.28 (s, 6H), 1.50 (d, J = 6.1 Hz, 3H), 1.16 (s, 3H). |
| 146 | 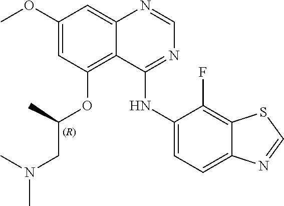 | (R)-N-(5-((1-(dimethylamino)propan-2-yl)oxy)-7- methoxyquinazolin-4-yl)-7-fluorobenzo[d]thiazol-6-amine LC-MS: (ES, m/z): RT = 0.971 min, LCMS: m/z = 428 [M + 1]; ¹H NMR (400 MHz, DMSO-d6) δ 10.37 (s, 1H), 9.41 (d, J = 1.5 Hz, 1H), 8.40 (d, J = 4.4 Hz, 1H), 8.35-8.21 (m, 1H), 7.98 (d, J = 8.8 Hz, 1H), 6.81 (q, J = 2.4 Hz, 2H), 4.90 (s, 1H), 3.90 (s, 3H), 2.82 (s, 1H), 2.41 (d, J = 12.7 Hz, 1H), 2.13 (s, 6H), 1.43 (d, J = 6.0 Hz, 3H). |
| 147 148 | 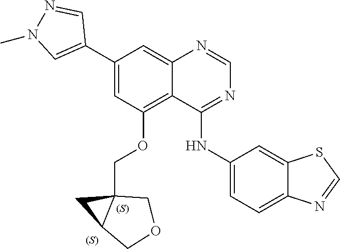 or 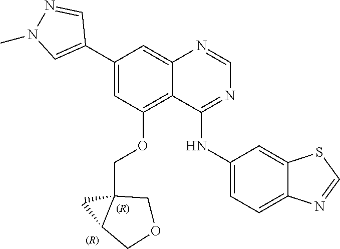 | N-(5-(((1S,5S)-3-oxabicyclo[3.1.0]hexan-1-yl)methoxy)-7-(1- methyl-1H-pyrazol-4-yl)quinazolin-4-yl)benzo[d]thiazol-6-amine OR N-(5-(((1R,5R)-3-oxabicyclo[3.1.0]hexan-1-yl)methoxy)-7-(1- methyl-1H-pyrazol-4-yl)quinazolin-4-yl)benzo[d]thiazol-6-amine First Eluting Isomer: LC-MS: (ES, m/z): RT = 0.821 min, LCMS: m/z = 471.30 [M + 1], Chiral-HPLC (ES): RT = 3.786 min, ¹H NMR (400 MHz, DMSO-d6) δ 10.67 (s, 1H), 9.42 (s, 1H), 8.76 (q, J = 3.0, 2.0 Hz, 2H), 8.51 (s, 1H), 8.23-8.15 (m, 2H), 7.71 (dd, J = 8.8, 2.2 Hz, 1H), 7.53 (d, J = 2.5 Hz, 2H), 4.87 (d, J = 10.7 Hz, 1H), 4.63 (d, J = 10.8 Hz, 1H), 3.93 (d, J = 8.3 Hz, 4H), 3.85 (d, J = 8.3 Hz, 1H), 3.77 (d, J = 8.4 Hz, 1H), 3.67 (dd, J = 8.4, 2.9 Hz, 1H), 1.89 (ddd, J = 7.7, 4.4, 2.8 Hz, 1H), 1.24 (s, 2H), 1.21 (s, 1H), 1.20-1.07 (m, 1H), 0.76 (t, J = 4.5 Hz, 1H),-0.06 (s, 0H). Second Eluting Isomer: LC-MS: (ES, m/z): RT = 0.813 min, LCMS: m/z = 471.30 [M + 1], Chiral-HPLC (ES): RT = 4.367 min, ¹H NMR (400 MHz, DMSO-d6) δ 10.24 (s, 1H), 9.33 (s, 1H), 8.91 (dd, J = 5.0, 2.1 Hz, 1H), 8.57 (d, J = 1.7 Hz, 1H), 8.47 (s, 1H), 8.18-8.10 (m, 2H), 7.66 (dd, J = 8.8, 2.2 Hz, 1H), 7.59 (d, J = 1.5 Hz, 1H), 7.38 (d, J = 1.6 Hz, 1H), 4.81 (d, J = 10.5 Hz, 1H), 4.53 (d, J = 10.4 Hz, 1H), 3.97 (d, J = 8.3 Hz, 1H), 3.91 (s, 3H), 3.87 (d, J = 8.3 Hz, 1H), 3.79 (d, J = 8.4 Hz, 1H), 3.72 (dd, J = 8.4, 2.9 Hz, 1H), 2.00 (q, J = 7.1, 6.7 Hz, 1H), 1.89 (ddd, J = 7.7, 4.4, 2.8 Hz, 1H), 1.24 (s, 3H), 1.12 (dd, J = 8.1, 4.4 Hz, 1H), 0.96-0.76 (m, 1H). |
| 149 150 | 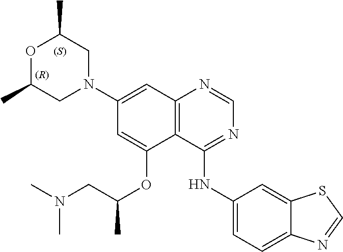 or 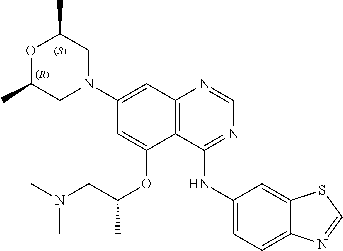 | N-(5-(((S)-1-(dimethylamino)propan-2-yl)oxy)-7-((2S,6R)-2,6- dimethylmorpholino)quinazolin-4-yl)benzo[d]thiazol-6-amine OR N-(5-(((R)-1-(dimethylamino)propan-2-yl)oxy)-7-((2S,6R)-2,6- dimethylmorpholino)quinazolin-4-yl)benzo[d]thiazol-6-amine First Eluting Isomer: LC-MS: (ES, m/z): RT = 0.707 min, LCMS: m/z = 493.40 [M + 1], Chiral-HPLC (ES): RT = 1.673, ¹H NMR (400 MHz, DMSO-d₆) δ 10.38 (s, 1H), 9.29 (s, 1H), 8.81 (d, J = 2.1 Hz, 1H), 8.41 (s, 1H), 8.08 (d, J = 8.8 Hz, 1H), 7.77 (dd, J = 8.8, 2.2 Hz, 1H), 6.88 (d, J = 2.2 Hz, 1H), 6.67 (d, J = 2.2 Hz, 1H), 5.06 (s, 1H), 3.89 (d, J = 12.2 Hz, 2H), 3.76-3.67 (m, 2H), 3.04 (s, 1H), 2.45 (ddd, J = 13.5, 10.5, 3.5 Hz, 2H), 2.29 (s, 6H), 1.47 (d, J = 5.9 Hz, 3H), 1.21 (d, J = 6.2 Hz, 6H). Second Eluting Isomer: LC-MS: (ES, m/z): RT = 0.703 min, LCMS: m/z = 493.40 [M + 1], Chiral-HPLC (ES): RT = 2.207, ¹H NMR (400 MHz, DMSO-d₆) δ 10.41 (s, 1H), 9.29 (s, 1H), 8.81 (d, J = 2.1 Hz, 1H), 8.41 (s, 1H), 8.08 (d, J = 8.8 Hz, 1H), 7.77 (dd, J = 8.8, 2.2 Hz, 1H), 6.88 (d, J = 2.2 Hz, 1H), 6.66 (d, J = 2.2 Hz, 1H), 5.04 (s, 1H), 3.82-4.00 (m, 2H), 3.89 (d, J = 12.2 Hz, 2H), 3.00 (s, 1H), 2.44 (ddd, J = 13.5, 10.5, 3.5 Hz, 3H), 2.26 (s, 6H), 1.47 (d, J = 5.9 Hz, 3H), 1.21 (d, J = 6.2 Hz, 6H). |
| 151 152 | 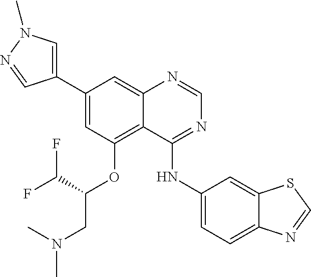 [SMILES: CN(C)C[C@@H](Oc1cc(-c2cnn(C)c2)cc2ncnc(Nc3ccc4ncsc4c3)c12)C(F)F] or 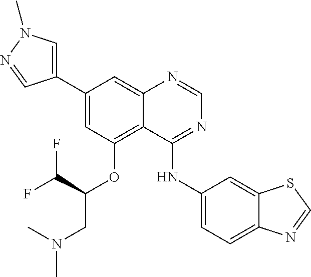 [SMILES: CN(C)C[C@H](Oc1cc(-c2cnn(C)c2)cc2ncnc(Nc3ccc4ncsc4c3)c12)C(F)F] | (R)-N-(5-((3-(dimethylamino)-1,1-difluoropropan-2-yl)oxy)-7-(1- methyl-1H-pyrazol-4-yl)quinazolin-4-yl)benzo[d]thiazol-6-amine, OR (S)-N-(5-((3-(dimethylamino)-1,1-difluoropropan-2-yl)oxy)-7- (1-methyl-1H-pyrazol-4-yl)quinazolin-4-yl)benzo[d]thiazol-6-amine First Eluting Isomer: LCMS: m/z = 496.15 [M + 1], Chiral-HPLC (ES): RT = 2.836 min, ¹H NMR (400 MHz, DMSO-d6) δ 10.25 (s, 1H), 9.33 (s, 1H), 8.89 (d, J = 2.1 Hz, 1H), 8.58 (s, 1H), 8.44 (s, 1H), 8.16-8.09 (m, 2H), 7.74 (dd, J = 8.9, 2.2 Hz, 1H), 7.66-7.61 (m, 2H), 6.64 (d, J = 2.3 Hz, 1H), 5.60 (s, 1H), 3.93 (s, 3H), 3.04 (s, 1H), 2.81 (d, J = 13.5 Hz, 1H), 2.30 (s, 6H), 2.08 (s, 1H). Second Eluting isomer. LC-MS: (ES, m/z): RT = 0.682 min, LCMS: m/z = 496.15 [M + 1], Chiral-HPLC (ES): RT = 3.776 min, ¹H NMR (400 MHz, DMSO-d6) δ 10.25 (s, 1H), 9.33 (s, 1H), 8.90 (d, J = 2.1 Hz, 1H), 8.58 (s, 1H), 8.44 (s, 1H), 8.17-8.09 (m, 2H), 7.74 (dd, J = 8.8, 2.2 Hz, 1H), 7.67-7.61 (m, 2H), 6.64 (d, J = 2.3 Hz, 1H), 5.61 (s, 1H), 3.93 (s, 3H), 3.06 (s, 1H), 2.84 (s, 1H), 2.31 (s, 6H), 2.08 (s, 1H). |
| 153 154 | 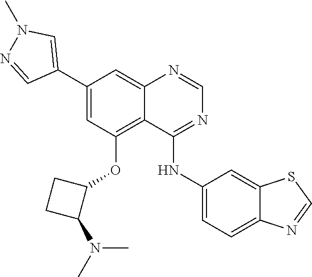 or 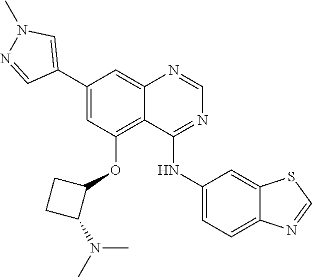 | N-(5-((1S,2S)-2-(dimethylamino)cyclobutoxy)-7-(1-methyl-1H- pyrazol-4-yl)quinazolin-4-yl)benzo[d]thiazol-6-amine OR N-(5- ((1R,2R)-2-(dimethylamino)cyclobutoxy)-7-(1-methyl-1H-pyrazol- 4-yl)quinazolin-4-yl)benzo[d]thiazol-6-amine First Eluting Compound: LC-MS: (ES, m/z): RT = 0.806 min, LCMS: m/z = 472.30 [M + 1], Chiral-HPLC (ES): RT = 1.416 min, ¹H NMR (400 MHz, DMSO-d6) δ 10.22 (s, 1H), 9.33 (s, 1H), 8.96 (d, J = 2.1 Hz, 1H), 8.57 (s, 1H), 8.47 (s, 1H), 8.17-8.11 (m, 2H), 7.79 (dd, J = 8.8, 2.2 Hz, 1H), 7.59 (d, J = 1.5 Hz, 1H), 7.27 (d, J = 1.6 Hz, 1H), 4.97 (q, J = 7.2 Hz, 1H), 3.92 (s, 3H), 2.62 (q, J = 8.9 Hz, 1H), 2.67 (s,1H), 2.26 (s, 6H), 2.08 (q, J = 9.4 Hz, 1H), 1.82 (p, J = 10.0 Hz, 1H), 1.60 (p, J = 9.6 Hz, 1H). Second Eluting Compound: LC-MS: (ES, m/z): RT = 0.806 min, LCMS: m/z = 472.30 [M + 1], Chiral-HPLC (ES): RT = 2.006 min, ¹H NMR (400 MHz, DMSO-d6) δ 10.20 (s, 1H), 9.33 (s, 1H), 8.95 (d, J = 2.1 Hz, 1H), 8.57 (s, 1H), 8.47 (s, 1H), 8.17-8.11 (m, 2H), 7.80 (dd, J = 8.8, 2.2 Hz, 1H), 7.59 (d, J = 1.4 Hz, 1H), 7.26 (d, J = 1.6 Hz, 1H), 4.98 (d, J = 7.4 Hz, 1H), 3.92 (s, 3H), 3.34 (s,1H), 2.63 (d, J = 9.4 Hz, 1H), 2.28 (s, 6H), 2.09 (q, J = 9.4 Hz, 1H), 1.82 (p, J = 10.0 Hz, 1H), 1.62 (t, J = 9.9 Hz, 1H). |
| 155 156 | 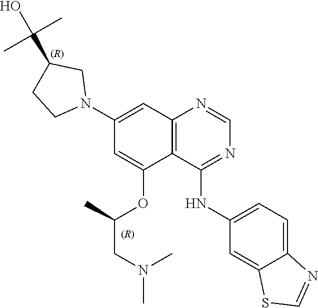 or 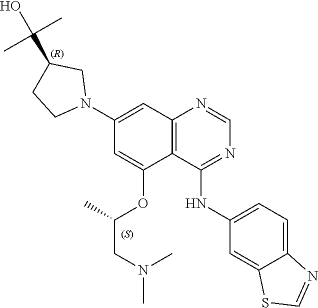 | 2-((R)-1-(4-(benzo[d]thiazol-6-ylamino)-5-(((R)-1- (dimethylamino)propan-2-yl)oxy)quinazolin-7-yl)pyrrolidin-3- yl)propan-2-01 OR 2-((R)-1-(4-(benzo[d]thiazol-6-ylamino)-5- (((S)-1-(dimethylamino)propan-2-yl)oxy)quinazolin-7-yl)pyrrolidin- 3-yl)propan-2-ol First Eluting Isomer: LC-MS: (ES, m/z): RT = 0.656 min, LCMS: m/z = 507.20 [M + 1], Chiral-HPLC (ES): RT = 3.930, ¹H NMR (400 MHz, DMSO-d6) δ 10.36 (s, 1H), 9.28 (s, 1H), 8.80 (d, J = 2.1 Hz, 1H), 8.34 (s, 1H), 8.07 (d, J = 8.8 Hz, 1H), 7.76 (dd, J = 8.8, 2.2 Hz, 1H), 6.46 (d, J = 2.1 Hz, 1H), 6.26 (d, J = 2.0 Hz, 1H), 5.01 (s, 1H), 4.42 (s, 1H), 3.52-3.61 (m, 1H), 3.57 (t, J = 9.0 Hz, 1H), 3.45 (t, J = 9.1 Hz, 2H), 3.06-2.96 (m, 1H), 2.37 (d, J = 9.4 Hz, 8H), 2.04- 1.96 (m, 2H), 1.49 (d, J = 5.9 Hz, 3H), 1.19 (d, J = 8.5 Hz, 6H). Second Eluting Isomer: LC-MS: (ES, m/z): RT = 1.532 min, LCMS: m/z = 507.20 [M + 1], Chiral-HPLC (ES): RT = 4.690, ¹H NMR (400 MHz, DMSO-d6) δ 10.36 (s, 1H), 9.28 (s, 1H), 8.81 (d, J = 2.1 Hz, 1H), 8.34 (s, 1H), 8.07 (d, J = 8.8 Hz, 1H), 7.76 (dd, J = 8.8, 2.2 Hz, 1H), 6.47 (d, J = 2.2 Hz, 1H), 6.26 (d, J = 2.1 Hz, 1H), 5.02-4.93 (m, 1H), 4.42 (s, 1H), 3.54 (t, J = 9.0 Hz, 1H), 3.46-3.37 (m, 2H), 2.98 (dd, J = 12.9, 8.5 Hz, 1H), 2.51-2.37 (m, 1H), 2.36 (s, 1H), 2.25 (s, 7H), 1.95 (dd, J = 20.4, 9.5 Hz, 2H), 1.50 (d, J = 6.0 Hz, 3H), 1.18 (d, J = 7.3 Hz, 6H). |
| 157 158 | 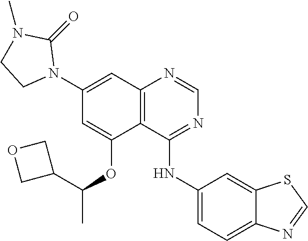 [SMILES: C[C@H](Oc1cc(N2CCN(C)C2=O)cc2ncnc(Nc3ccc4ncsc4c3)c12)C1COC1] or 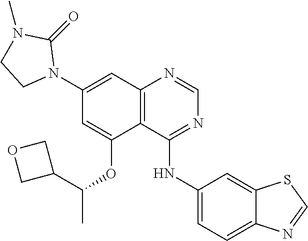 [SMILES: C[C@@H](Oc1cc(N2CCN(C)C2=O)cc2ncnc(Nc3ccc4ncsc4c3)c12)C1COC1] | (S)-1-(4-(benzo[d]thiazol-6-ylamino)-5-(1-(oxetan-3- yl)ethoxy)quinazolin-7-yl)-3-methylimidazolidin-2-one OR (R)-1- (4-(benzo[d]thiazol-6-ylamino)-5-(1-(oxetan-3- yl)ethoxy)quinazolin-7-yl)-3-methylimidazolidin-2-one First Eluting Isomer: LC-MS: (ES, m/z): RT = 0.984 min, LCMS: m/z = 477.35 [M + 1], Chiral-HPLC (ES): RT = 2.589, ¹H NMR (300 MHz, DMSO-d6) δ 10.02 (s, 1H), 9.32 (s, 1H), 8.73 (d, J = 2.1 Hz, 1H), 8.48 (s, 1H), 8.15 (d, J = 2.0 Hz, 1H), 8.07 (d, J = 8.7 Hz, 1H), 7.77 (dd, J = 8.8, 2.2 Hz, 1H), 7.05 (d, J = 2.0 Hz, 1H), 5.07 (t, J = 6.2 Hz, 1H), 4.83 (q, J = 7.3 Hz, 2H), 4.64 (t, J = 6.0 Hz, 2H), 3.95 (q, J = 7.4, 6.5 Hz, 2H), 3.53 (t, J = 7.9 Hz, 3H), 2.84 (s, 3H), 1.43 (d, J = 6.0 Hz, 3H). Second Eluting Isomer: LC-MS: (ES, m/z): RT = 0.979 min, LCMS: m/z = 477.35 [M + 1], Chiral-HPLC (ES): RT = 3.102, ¹H NMR (300 MHz, DMSO-d6) δ 10.02 (s, 1H), 9.32 (s, 1H), 8.73 (d, J = 2.2 Hz, 1H), 8.48 (s, 1H), 8.15 (d, J = 2.1 Hz, 1H), 8.07 (d, J = 8.7 Hz, 1H), 7.77 (dd, J = 8.8, 2.2 Hz, 1H), 7.05 (d, J = 2.0 Hz, 1H), 5.07 (t, J = 6.1 Hz, 1H), 4.83 (q, J = 7.6 Hz, 2H), 4.64 (t, J = 6.2 Hz, 2H), 4.02- 3.89 (m, 2H), 3.53 (t, J = 7.9 Hz, 3H), 2.84 (s, 3H), 1.43 (d, J = 6.0 Hz, 3H). |
| 159 160 | 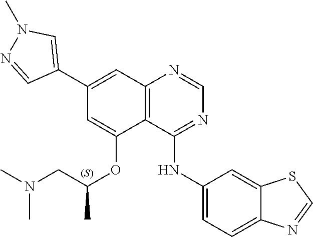 or 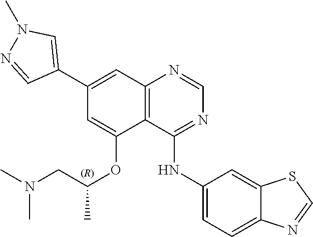 | (R)-N-(5-((1-(dimethylamino)propan-2-yl)oxy)-7-(1-methyl-1H- pyrazol-4-yl)quinazolin-4-yl)benzo[d]thiazol-6-amine OR (S)-N- (5-((1-(dimethylamino)propan-2-yl)oxy)-7-(1-methyl-1H-pyrazol-4- yl)quinazolin-4-yl)benzo[d]thiazol-6-amine First Eluting Isomer: LC-MS: (ES, m/z): RT-0.630 min, LCMS: m/z = 460.35 [M + 1], Chiral-HPLC (ES): RT = 1.962, ¹H NMR (300 MHz, DMSO-d6) δ 10.62 (s, 1H), 9.32 (s, 1H), 8.83 (dd, J = 5.1, 2.1 Hz, 1H), 8.53 (d, J = 2.0 Hz, 2H), 8.18-8.06 (m, 2H), 7.80 (dd, J = 8.8, 2.1 Hz, 1H), 7.55 (d, J = 1.4 Hz, 1H), 7.39 (d, J = 1.5 Hz, 1H), 5.11 (s, 1H), 3.92 (s, 3H), 3.34 (s,1H), 3.10-2.96 (m, 1H), 2.26 (s, 6H), 1.51 (d, J = 5.9 Hz, 3H). Second Eluting Isomer: LC-MS: (ES, m/z): RT = 0.628 min, LCMS: m/z = 460.35 [M + 1], Chiral-HPLC (ES): RT = 2.696, ¹H NMR (300 MHz, DMSO-d6) δ 10.61 (s, 1H), 9.32 (s, 1H), 8.83 (dd, J = 5.1, 2.1 Hz, 1H), 8.53 (s, 2H), 8.18-8.07 (m, 2H), 7.80 (dt, J = 8.8, 1.6 Hz, 1H), 7.55 (d, J = 1.4 Hz, 1H), 7.39 (d, J = 1.6 Hz, 1H), 5.12 (s, 1H), 3.92 (s, 3H), 3.37 (s,1H), 3.04 (s, 1H), 2.27 (d, J = 4.2 Hz, 6H), 1.51 (d, J = 5.9 Hz, 3H). |
| 161 162 | 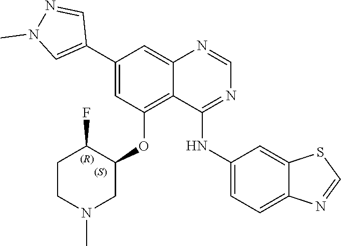 or 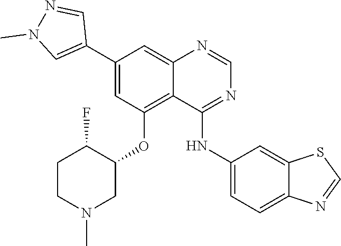 [SMILES: CN1CC[C@H](F)[C@H](Oc2cc(-c3cnn(C)c3)cc3ncnc(Nc4ccc5ncsc5c4)c23)C1] | N-(5-(((3R,4S)-4-fluoro-1-methylpiperidin-3-yl)oxy)-7-(1-methyl- 1H-pyrazol-4-yl)quinazolin-4-yl)benzo[d]thiazol-6-amine OR N- (5-(((3S,4R)-4-fluoro-1-methylpiperidin-3-yl)oxy)-7-(1-methyl-1H- pyrazol-4-yl)quinazolin-4-yl)benzo[d]thiazol-6-amine First Eluting Isomer: LC-MS: (ES, m/z): RT = 1.518 min, LCMS: m/z = 490 [M + 1], Chiral-HPLC (ES): RT = 2.431, ¹H NMR (400 MHz,DMSO-d6) δ 10.62 (s, 1H), 9.32 (s, 1H), 8.95 (d, J = 2.1 Hz, 1H), 8.57 (s, 1H), 8.45 (s, 1H), 8.12 (d, J = 9.9 Hz, 2H), 7.81 (dd, J = 8.9, 2.2 Hz, 1H), 7.64-7.52 (m, 2H), 5.48 (d, J = 14.3 Hz, 1H), 5.15 (d, J = 48.0 Hz, 1H), 3.92 (s, 3H),2.79 (d, J = 74.8 Hz, 3H), 2.47- 2.41 (m, 1H), 2.26 (s, 3H), 2.21-1.94 (m, 2H). Second Eluting Isomer: LC-MS: (ES, m/z): RT = 1.516 min, LCMS: m/z = 490 [M + 1], Chiral-HPLC (ES): RT = 2.915, ¹H NMR (400 MHz, DMSO-d6) δ 10.62 (s, 1H), 9.32 (s, 1H), 8.95 (d, J = 2.1 Hz, 1H), 8.57 (s, 1H), 8.45 (s, 1H), 8.13 (d, J = 9.8 Hz, 2H), 7.82 (dd, J = 8.9, 2.1 Hz, 1H), 7.63-7.52 (m, 2H), 5.48 (d, J = 14.5 Hz, 1H), 5.15 (d, J = 48.2 Hz, 1H), 3.92 (s, 3H), 2.79 (d, J = 76.8 Hz, 3H), 2.46- 2.41 (m, 1H), 2.26 (s, 3H), 2.25-1.94 (m, 2H). |
| 163 | 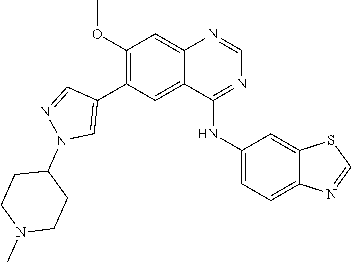 [SMILES: COc1cc2ncnc(Nc3ccc4ncsc4c3)c2cc1-c1cnn(C2CCN(C)CC2)c1] | N-(7-methoxy-6-(1-(1-methylpiperidin-4-yl)-1H-pyrazol-4- yl)quinazolin-4-yl)benzo[d]thiazol-6-amine LC-MS: (ES, m/z): RT = 1.222 min, LCMS: m/z = 472 [M + 1], ¹H- NMR 1H NMR (300 MHz, DMSO-d6) δ 9.83 (s, 1H), 9.30 (s, 1H), 8.81-8.65 (m, 2H), 8.53 (s, 1H), 8.28 (s, 1H), 8.18-8.04 (m, 2H), 7.90 (dd, J = 8.9, 2.2 Hz, 1H), 7.26 (s, 1H), 4.19 (s, 1H), 4.03 (s, 3H), 2.87 (d, J = 5.8 Hz, 2H), 2.21 (s, 3H), 2.10-1.94 (m, 6H). |
| 164 | 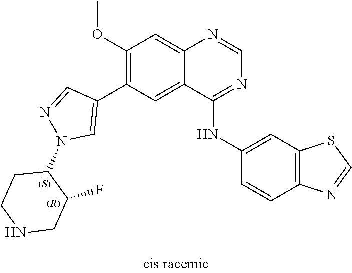 | cis-N-(6-(1-((3R,4S)-3-fluoropiperidin-4-yl)-1H-pyrazol-4-yl)-7- methoxyquinazolin-4-yl)benzo[d]thiazol-6-amine LC-MS: (ES, m/z): RT = 2.451 min, LCMS: m/z = 476.1 [M + 1], ¹H NMR (300 MHz, DMSO-d6) δ 9.86 (s, 1H), 9.31 (s, 2H), 8.80-8.71 (m, 4H), 8.55 (d, J = 1.4 Hz, 2H), 8.26 (s, 2H), 8.19 (s, 2H), 8.11 (d, J = 8.8 Hz, 2H), 7.92 (dt, J = 8.9, 2.6 Hz, 2H), 7.29 (s, 2H), 5.01 (s, 1H), 4.84 (s, 1H), 4.69-4.58 (m, 1H), 4.53 (ddd, J = 12.4, 4.6, 2.0 Hz, 1H), 4.05 (s, 6H), 3.22-3.06 (m, 4H), 2.91 (d, J = 14.5 Hz, 1H), 2.78 (d, J = 14.3 Hz, 1H), 2.68 (s, 2H), 2.54 (s, 33H), 2.20 (d, J = 4.3 Hz, 1H), 1.96 (dd, J = 12.8, 3.9 Hz, 2H). |
| 165 | 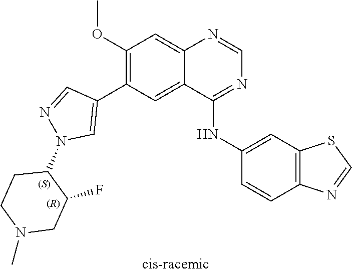 | cis-N-(6-(1-((3R,4S)-3-fluoro-1-methylpiperidin-4-yl)-1H-pyrazol- 4-yl)-7-methoxyquinazolin-4-yl)benzo[d]thiazol-6-amine LC-MS: (ES, m/z): RT = 2.641 min, LCMS: m/z = 490.1 [M + 1], ¹H NMR (300 MHz, DMSO-d6) δ 9.31 (s, 2H), 8.79-8.71 (m, 4H), 8.55 (d, J = 1.5 Hz, 2H), 8.28 (s, 2H), 8.19 (s, 2H), 8.11 (d, J = 8.8 Hz, 2H), 7.92 (dt, J = 8.5, 2.6 Hz, 2H), 7.29 (s, 2H), 5.13 (s, 1H), 4.96 (s, 1H), 4.52 (d, J = 12.6 Hz, 1H), 4.44 (s, 1H), 4.05 (s, 6H), 3.13 (t, J = 11.1 Hz, 3H), 2.97 (d, J = 11.3 Hz, 3H), 2.67 (s, 18H), 2.54 (d, J = 3.0 Hz, 17H), 2.24 (s, 6H), 2.18 (d, J = 11.6 Hz, 2H), 2.10-1.99 (m, 2H). |
| 166 167 | 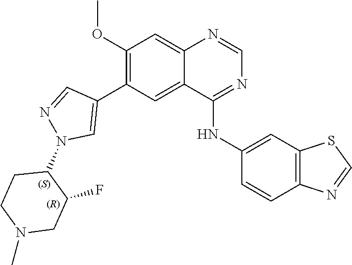 or 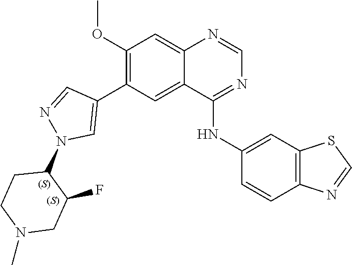 | N-(6-(1-((3R,4S)-3-fluoro-1-methylpiperidin-4-yl)-1H-pyrazol-4- yl)-7-methoxyquinazolin-4-yl)benzo[d]thiazol-6-amine OR N-(6- (1-((3S,4S)-3-fluoro-1-methylpiperidin-4-yl)-1H-pyrazol-4-yl)-7- methoxyquinazolin-4-yl)benzo[d]thiazol-6-amine First Eluting Isomer: LC-MS: (ES, m/z): RT = 1.354 min, LCMS: m/z = 490.1 [M + 1], ¹H NMR (400 MHz, Methanol-d4) δ 9.21 (s, 1H), 8.74-8.66 (m, 2H), 8.52 (s, 1H), 8.35 (d, J = 1.2 Hz, 1H), 8.23 (d, J = 0.6 Hz, 1H), 8.10 (d, J = 8.8 Hz, 1H), 7.88 (dd, J = 8.8, 2.1 Hz, 1H), 7.28 (s, 1H), 5.06 (d, J = 49.4 Hz, 1H), 4.54-4.30 (m, 1H), 4.11 (s, 3H), 3.13 (d, J = 11.2 Hz, 1H), 2.68-2.50 (m, 1H), 2.40 (dd, J = 29.5, 12.6 Hz, 1H), 2.40 (s, 3H), 2.16 (d, J = 11.8 Hz, 1H). Second Eluting Isomer: LC-MS: (ES, m/z): RT = 1.349 min, LCMS: m/z = 490.1 [M + 1], ¹H NMR (400 MHz, Methanol-d4) δ 9.21 (s, 1H), 8.74-8.65 (m, 2H), 8.51 (s, 1H), 8.35 (d, J = 1.1 Hz, 1H), 8.23 (d, J = 0.6 Hz, 1H), 8.10 (d, J = 8.8 Hz, 1H), 7.88 (dd, J = 8.8, 2.2 Hz, 1H), 7.27 (s, 1H), 5.06 (d, J = 49.2 Hz, 1H), 4.49 (dd, J = 30.2, 12.5 Hz, 1H), 4.11 (s, 3H), 3.13 (d, J = 12.2 Hz, 1H), 2.68- 2.50 (m, 2H), 2.40 (dd, J = 29.7, 12.9 Hz, 1H), 2.40 (s, 3H), 2.20- 2.12 (m, 1H). |
| 168 | 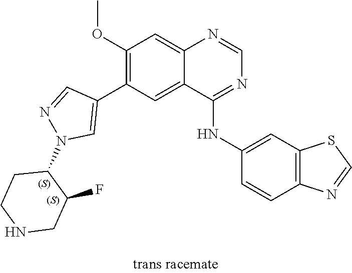 | trans-N-(6-(1-((3S,4S)-3-fluoropiperidin-4-yl)-1H-pyrazol-4-yl)-7- methoxyquinazolin-4-yl)benzo[d]thiazol-6-amine LC-MS: (ES, m/z): RT = 1.139 min, LCMS: m/z = 476 [M + 1]. ¹H NMR (300 MHz, DMSO-d6) δ 9.87 (s, 1H), 9.30 (s, 1H), 8.74 (d, J = 3.0 Hz, 2H), 8.53 (s, 1H), 8.33 (s, 1H), 8.20 (s, 1H), 8.10 (d, J = 8.8 Hz, 1H), 7.90 (dd, J = 8.8, 2.1 Hz, 1H), 7.28 (s, 1H), 5.00-4.59 (m, 1H), 4.45 (t, J = 9.1 Hz, 1H), 4.04 (s, 3H), 2.96 (d, J = 12.8 Hz, 1H), 2.55 (d, J = 10.7 Hz, 3H), 2.01 (d, J = 4.2 Hz, 2H). |
| 169 | 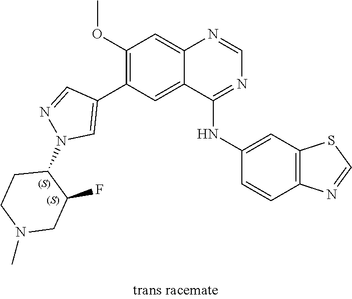 | trans-N-(6-(1-((3S,4S)-3-fluoro-1-methylpiperidin-4-yl)-1H- pyrazol-4-yl)-7-methoxyquinazolin-4-yl)benzo[d]thiazol-6-amine LC-MS: (ES, m/z): RT = 1.222 min, LCMS: m/z = 490 [M + 1]. ¹H NMR (400 MHz, DMSO-d6) δ 9.86 (s, 1H), 9.30 (s, 1H), 8.82-8.67 (m, 2H), 8.54 (s, 1H), 8.37 (s, 1H), 8.20 (s, 1H), 8.10 (d, J = 8.8 Hz, 1H), 7.91 (dd, J = 8.9, 2.2 Hz, 1H), 7.28 (s, 1H), 4.95 (dtd, J = 50.1, 9.8, 5.1 Hz, 1H), 4.52-4.35 (m, 1H), 4.05 (s, 3H), 3.27-3.19 (m, 1H), 2.84 (d, J = 8.7 Hz, 1H), 2.29 (s, 3H), 2.18-1.97 (m, 4H). |
| 170 171 | 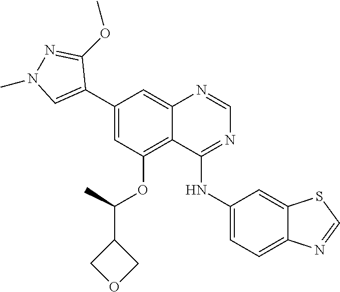 [SMILES: COc1nn(C)cc1-c1cc(O[C@H](C)C2COC2)c2c(Nc3ccc4ncsc4c3)ncnc2c1] or 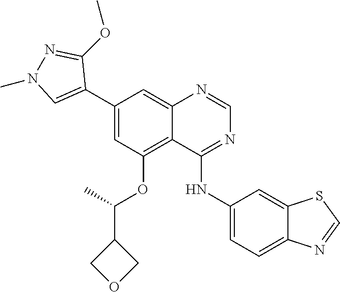 [SMILES: COc1nn(C)cc1-c1cc(O[C@@H](C)C2COC2)c2c(Nc3ccc4ncsc4c3)ncnc2c1] | (S)-N-(7-(3-methoxy-1-methyl-1H-pyrazol-4-yl)-5-(1-(oxetan-3- yl)ethoxy)quinazolin-4-yl)benzo[d]thiazol-6-amine OR (R)-N-(7- (3-methoxy-1-methyl-1H-pyrazol-4-yl)-5-(1-(oxetan-3- yl)ethoxy)quinazolin-4-yl)benzo[d]thiazol-6-amine First Eluting Isomer: LC-MS: (ES, m/z): RT = 1.081 min, LCMS: m/z = 489 [M + 1], Chiral-HPLC (ES): RT = 3.174, ¹H NMR (400 MHz, DMSO-d6) δ 10.27 (s, 1H), 9.34 (s, 1H), 8.69 (d, J = 2.1 Hz, 1H), 8.56 (s, 1H), 8.41 (s, 1H), 8.10 (d, J = 8.7 Hz, 1H), 7.80-7.64 (m, 2H), 7.49 (d, J = 1.5 Hz, 1H), 5.29 (p, J = 6.1 Hz, 1H), 4.85 (td, J = 8.2, 6.3 Hz, 2H), 4.61 (dt, J = 38.3, 6.0 Hz, 2H), 3.99 (s, 3H), 3.79 (s, 3H), 3.52 (p, J = 7.4, 6.9 Hz, 1H), 1.43 (d, J = 5.9 Hz, 3H). Second Eluting Isomer: LC-MS: (ES, m/z): RT = 1.072 min, LCMS: m/z = 489 [M + 1], Chiral-HPLC (ES): RT = 3.766, ¹H NMR (400 MHz, DMSO-d6) δ 10.27 (s, 1H), 9.34 (s, 1H), 8.69 (d, J = 2.1 Hz, 1H), 8.56 (s, 1H), 8.41 (s, 1H), 8.10 (d, J = 8.7 Hz, 1H), 7.80-7.64 (m, 2H), 7.49 (d, J = 1.5 Hz, 1H), 5.29 (p, J = 6.1 Hz, 1H), 4.85 (td, J = 8.2, 6.3 Hz, 2H), 4.61 (dt, J = 38.3, 6.0 Hz, 2H), 3.99 (s, 3H), 3.79 (s, 3H), 3.52 (p, J = 7.4, 6.9 Hz, 1H), 1.43 (d, J = 5.9 Hz, 3H). |
| 172 173 | 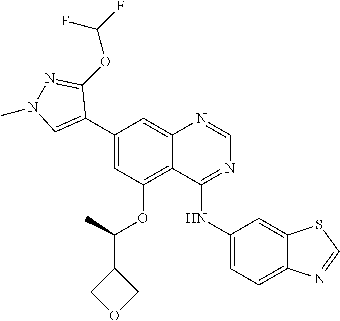 [SMILES: C[C@@H](Oc1cc(-c2cn(C)nc2OC(F)F)cc2ncnc(Nc3ccc4ncsc4c3)c12)C1COC1] or 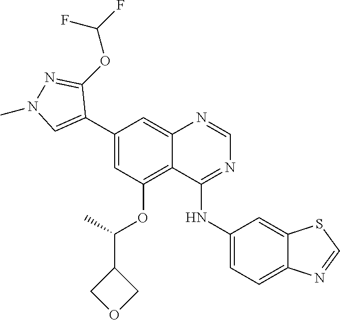 [SMILES: C[C@H](Oc1cc(-c2cn(C)nc2OC(F)F)cc2ncnc(Nc3ccc4ncsc4c3)c12)C1COC1] | (R)-N-(7-(3-(difluoromethoxy)-1-methyl-1H-pyrazol-4-yl)-5-(1- (oxetan-3-yl)ethoxy)quinazolin-4-yl)benzo[d]thiazol-6-amine OR (S)-N-(7-(3-(difluoromethoxy)-1-methyl-1H-pyrazol-4-yl)-5-(1- (oxetan-3-yl)ethoxy)quinazolin-4-yl)benzo[d]thiazol-6-amine First Eluting Isomer: LC-MS: (ES, m/z): RT = 1.197 min, LCMS: m/z = 525.15 [M + 1], Chiral-HPLC (ES): RT = 1.741, H-NMR 1H NMR (400 MHz, DMSO-d6) δ 10.13 (s, 1H), 9.34 (s, 1H), 8.74 (d, J = 2.1 Hz, 1H), 8.65-8.49 (m, 2H), 8.13-8.03 (m, 1H), 7.88-7.74 (m, 1H), 7.70-7.24 (m, 3H), 5.27 (p, J = 6.1 Hz, 1H), 4.85 (td, J = 8.1, 6.4 Hz, 2H), 4.61 (dt, J = 41.4, 6.0 Hz, 2H), 3.85 (d, J = 4.9 Hz, 3H), 3.54 (td, J = 7.6, 5.5 Hz, 1H), 1.44 (d, J = 6.0 Hz, 3H). Second Eluting Isomer: LC-MS: (ES, m/z): RT = 1.158 min, LCMS: m/z = 525.15 [M + 1], Chiral-HPLC (ES): RT = 1.862, ¹H-NMR 1H NMR (400 MHz, DMSO-d6) δ 10.13 (s, 1H), 9.33 (s, 1H), 8.74 (d, J = 2.1 Hz, 1H), 8.53 (d, J = 5.5 Hz, 2H), 8.09 (d, J = 8.8 Hz, 1H), 7.79 (dd, J = 8.8, 2.2 Hz, 1H), 7.67-7.24 (m, 3H), 5.27 (p, J = 6.1 Hz, 1H), 4.85 (td, J = 8.2, 6.4 Hz, 2H), 4.61 (dt, J = 41.4, 6.0 Hz, 2H), 3.86 (s, 3H), 3.54 (qd, J = 7.5, 7.1, 5.4 Hz, 1H), 1.44 (d, J = 6.0 Hz, 3H). |
| 174 175 | 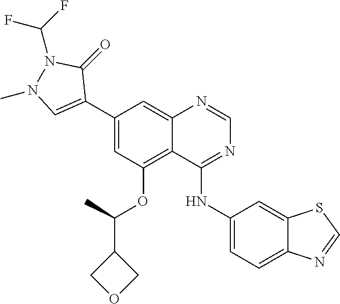 [SMILES: C[C@@H](Oc1cc(-c2cn(C)n(C(F)F)c2=O)cc2ncnc(Nc3ccc4ncsc4c3)c12)C1COC1] or 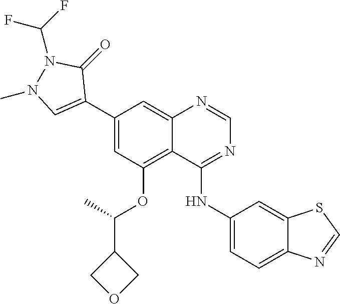 [SMILES: C[C@H](Oc1cc(-c2cn(C)n(C(F)F)c2=O)cc2ncnc(Nc3ccc4ncsc4c3)c12)C1COC1] | (R)-4-(4-(benzo[d]thiazol-6-ylamino)-5-(1-(oxetan-3- yl)ethoxy)quinazolin-7-yl)-2-(difluoromethyl)-1-methyl-1,2- dihydro-3H-pyrazol-3-one OR (S)-4-(4-(benzo[d]thiazol-6- ylamino)-5-(1-(oxetan-3-yl)ethoxy)quinazolin-7-yl)-2- (difluoromethyl)-1-methyl-1,2-dihydro-3H-pyrazol-3-one First Eluting Isomer: LC-MS: (ES, m/z): RT = 1.052 min, LCMS: m/z = 525 [M + 1], Chiral-HPLC (ES): RT = 2.004, ¹H NMR (300 MHz, DMSO-d6) δ 10.21 (s, 1H), 9.34 (s, 1H), 8.98 (s, 1H), 8.72 (s, 1H), 8.56 (s, 1H), 8.10 (d, J = 8.8 Hz, 1H), 7.94 (d, J = 1.4 Hz, 1H), 7.89- 7.45 (m, 3H), 5.24 (t, J = 6.1 Hz, 1H), 4.97-4.77 (m, 2H), 4.65 (t, J = 6.0 Hz, 1H), 4.55 (t, J = 6.0 Hz, 1H), 3.68 (s, 3H), 3.53 (d, J = 6.8 Hz, 1H), 1.44 (d, J = 6.0 Hz, 3H). Second Eluting Isomer: LC-MS: (ES, m/z): RT = 1.047 min, LCMS: m/z = 488 [M + 1], Chiral-HPLC (ES): RT = 2.690, ¹H NMR (300 MHz, DMSO-d6) δ 10.16 (s, 1H), 9.33 (s, 1H), 8.98 (s, 1H), 8.74 (d, J = 2.1 Hz, 1H), 8.54 (s, 1H), 8.09 (d, J = 8.8 Hz, 1H), 7.93 (d, J = 1.4 Hz, 1H), 7.89-7.45 (m, 3H), 5.24 (t, J = 6.2 Hz, 1H), 4.85 (dt, J = 7.9, 6.2 Hz, 2H), 4.65 (t, J = 6.1 Hz, 1H), 4.55 (t, J = 6.0 Hz, 1H), 3.68 (s, 3H), 3.53 (d, J = 6.5 Hz, 1H), 1.44 (d, J = 6.0 Hz, 3H). |
| 176 | 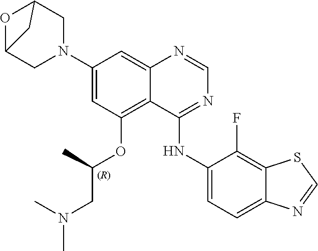 | N-(7-(6-oxa-3-azabicyclo[3.1.1]heptan-3-yl)-5-(((R)-1- (dimethylamino)propan-2-yl)oxy)quinazolin-4-yl)-7- fluorobenzo[d]thiazol-6-amine LC-MS: (ES, m/z): RT = 0.914 min, LCMS: m/z = 495.10 [M + 1]. ¹H NMR (400 MHz, DMSO-d6) δ 10.31 (s, 1H), 9.41 (d, J = 1.5 Hz, 1H), 8.41 (dd, J = 8.8, 7.8 Hz, 1H), 8.31 (s, 1H), 7.99 (d, J = 8.8 Hz, 1H), 6.70 (d, J = 2.3 Hz, 1H), 6.52 (d, J = 2.2 Hz, 1H), 5.02-4.95 (m, 1H), 4.77 (d, J = 6.4 Hz, 2H), 3.72 (dd, J = 11.7, 8.3 Hz, 2H), 3.61 (d, J = 11.6 Hz, 2H), 3.17 (q, J = 7.1 Hz, 1H), 2.84 (dd, J = 12.9, 8.1 Hz, 1H), 2.44 (dd, J = 12.9, 4.4 Hz, 1H), 2.17 (s, 6H), 1.93 (d, J = 8.8 Hz, 1H), 1.50 (d, J = 6.0 Hz, 3H). |
| 177 | 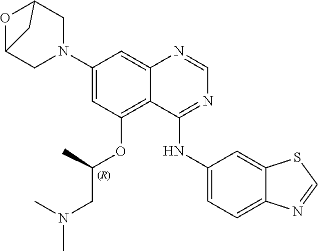 | N-(7-(6-oxa-3-azabicyclo[3.1.1]heptan-3-yl)-5-(((R)-1- (dimethylamino)propan-2-yl)oxy)quinazolin-4-yl)benzo[d]thiazol-6- amine LC-MS: (ES, m/z): RT = 0.629 min, LCMS: m/z = 477 [M + 1]. ¹H NMR (400 MHz, DMSO-d6) δ 10.41 (s, 1H), 9.29 (s, 1H), 8.82 (dd, J = 6.3, 2.1 Hz, 1H), 8.39 (d, J = 2.1 Hz, 1H), 8.08 (d, J = 8.8 Hz, 1H), 7.78 (dd, J = 8.8, 2.2 Hz, 1H), 6.68 (d, J = 2.2 Hz, 1H), 6.51 (d, J = 2.1 Hz, 1H), 5.04 (s, 1H), 4.78 (d, J = 6.4 Hz, 2H), 3.73 (dd, J = 18.1, 11.7 Hz, 4H), 3.66-3.57 (m, 1H), 3.18 (q, J = 7.3 Hz, 1H), 3.03 (s, 1H), 2.28 (s, 6H), 1.94 (d, J = 8.8 Hz, 1H), 1.51 (d, J = 5.9 Hz, 3H). |
| 178 179 | 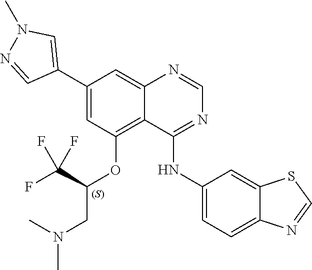 or 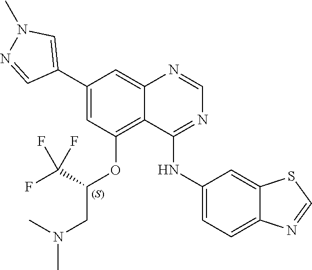 | (S)-N-(5-((3-(dimethylamino)-1,1,1-trifluoropropan-2-yl)oxy)-7-(1- methyl-1H-pyrazol-4-yl)quinazolin-4-yl)benzo[d]thiazol-6-amine OR (R)-N-(5-((3-(dimethylamino)-1,1,1-trifluoropropan-2-yl)oxy)- 7-(1-methyl-1H-pyrazol-4-yl)quinazolin-4-yl)benzo[d]thiazol-6- amine First Eluting Isomer: LC-MS: (ES, m/z): RT = 0.712 min, LCMS: m/z = 514.15 [M + 1], Chiral-HPLC (ES): RT = 2.333 min, ¹H NMR (400 MHz, DMSO-d6) δ 10.09 (s, 1H), 9.33 (s, 1H), 8.88 (d, J = 2.1 Hz, 1H), 8.59 (s, 1H), 8.44 (s, 1H), 8.18-8.09 (m, 2H), 7.75-7.66 (m, 3H), 6.06 (s, 1H), 3.93 (s, 3H), 3.22 (dd, J = 13.8, 8.2 Hz, 1H), 2.85 (dd, J = 13.8, 3.3 Hz, 1H), 2.28 (s, 6H). Second Eluting Isomer: LC-MS: (ES, m/z): RT = 0.714 min, LCMS: m/z = 514.35 [M + 1], Chiral-HPLC (ES): RT = 3.054 min, ¹H NMR (400 MHz, DMSO-d6) δ 10.09 (s, 1H), 9.33 (s, 1H), 8.88 (d, J = 2.1 Hz, 1H), 8.59 (s, 1H), 8.44 (s, 1H), 8.18-8.09 (m, 2H), 7.76-7.66 (m, 3H), 6.06 (s, 1H), 3.93 (s, 3H), 3.21 (dd, J = 13.8, 8.2 Hz, 1H), 2.85 (dd, J = 13.8, 3.3 Hz, 1H), 2.28 (s, 6H). |
| 180 | 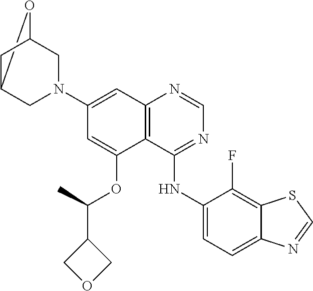 [SMILES: C[C@@H](Oc1cc(N2CC3CC(C2)O3)cc2ncnc(Nc3ccc4ncsc4c3F)c12)C1COC1] | N-(7-(6-oxa-3-azabicyclo[3.1.1]heptan-3-yl)-5-((R)-1-(oxetan-3- yl)ethoxy)quinazolin-4-yl)-7-fluorobenzo[d]thiazol-6-amine LC-MS: (ES, m/z): RT = 0.812 min, LCMS: m/z = 494.15 [M + 1], ¹H NMR (400 MHz, DMSO-d6) δ 9.92-9.87 (m, 1H), 9.41 (d, J = 1.5 Hz, 1H), 8.52 (dd, J = 8.9, 7.9 Hz, 1H), 8.34 (s, 1H), 7.98 (d, J = 8.9 Hz, 1H), 6.75 (d, J = 2.3 Hz, 1H), 6.55 (d, J = 2.1 Hz, 1H), 5.36 (p, J = 6.1 Hz, 1H), 4.80 (dd, J = 8.5, 6.3 Hz, 4H), 4.57 (t, J = 6.0 Hz, 1H), 4.49 (t, J = 5.9 Hz, 1H), 3.77 (d, J = 11.7 Hz, 2H), 3.63 (t, J = 10.6 Hz, 2H), 3.49-3.37 (m, 1H), 3.19 (q, J = 7.2 Hz, 1H), 1.95 (d, J = 8.8 Hz, 1H), 1.40 (d, J = 6.0 Hz, 3H), 1.23 (s, 1H). |
| 181 | 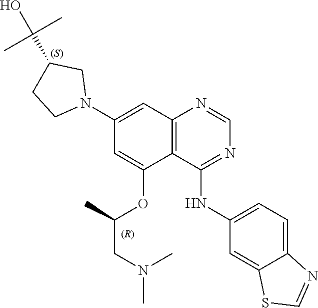 | 2-((S)-1-(4-(benzo[d]thiazol-6-ylamino)-5-(((R)-1- (dimethylamino)propan-2-yl)oxy)quinazolin-7-yl)pyrrolidin-3- yl)propan-2-ol LC-MS: (ES, m/z): RT = 0.792 min, LCMS: m/z = 507 [M + 1]; ¹H NMR (300 MHz, DMSO-d6) δ 10.37 (s, 1H), 9.28 (s, 1H), 8.81 (d, J = 2.1 Hz, 1H), 8.33 (s, 1H), 8.06 (d, J = 8.8 Hz, 1H), 7.75 (dd, J = 8.9, 2.2 Hz, 1H), 6.49-6.42 (m, 1H), 6.25 (d, J = 2.0 Hz, 1H), 4.99 (s, 1H), 4.42 (s, 1H), 3.57 (t, J = 8.8 Hz, 1H), 3.45 (t, J = 9.0 Hz, 1H), 3.39-3.34 (m, 1H), 3.30 (d, J = 9.1 Hz, 1H), 2.97 (dd, J = 12.9, 8.5 Hz, 1H), 2.49-2.29 (m, 2H), 2.24 (s, 6H), 1.97 (s, 2H), 1.49 (d, J = 5.9 Hz, 3H), 1.18 (d, J = 6.4 Hz, 6H). |
| 182 | 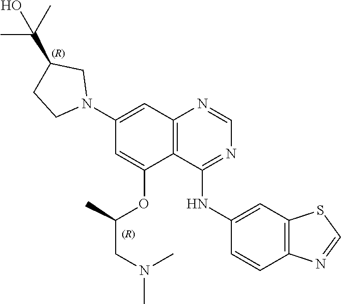 | 2-((R)-1-(4-(benzo[d]thiazol-6-ylamino)-5-(((R)-1- (dimethylamino)propan-2-yl)oxy)quinazolin-7-yl)pyrrolidin-3- yl)propan-2-ol LC-MS: (ES, m/z): RT = 0.795 min, LCMS: m/z = 507 [M + 1]; ¹H NMR (300 MHz, DMSO-d6) δ 10.37 (s, 1H), 9.28 (s, 1H), 8.81 (d, J = 2.1 Hz, 1H), 8.34 (s, 1H), 8.07 (d, J = 8.8 Hz, 1H), 7.76 (dd, J = 8.9, 2.2 Hz, 1H), 6.47 (d, J = 2.1 Hz, 1H), 6.26 (d, J = 1.9 Hz, 1H), 4.99 (d, J = 9.6 Hz, 1H), 4.42 (s, 1H), 3.54 (t, J = 8.9 Hz, 1H), 3.41 (t, J = 9.4 Hz, 1H), 3.30 (d, J = 9.3 Hz, 1H), 2.97 (dd, J = 12.9, 8.4 Hz, 1H), 2.47-2.30 (m, 2H), 2.24 (s, 6H), 2.03-1.87 (m, 2H), 1.50 (d, J = 5.9 Hz, 3H), 1.18 (d, J = 5.5 Hz, 6H). |
| 183 | 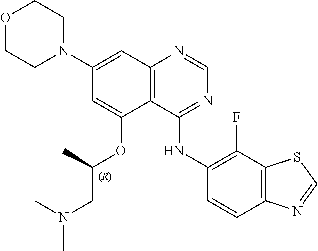 | (R)-N-(5-((1-(dimethylamino)propan-2-yl)oxy)-7- morpholinoquinazolin-4-yl)-7-fluorobenzo[d]thiazol-6-amine LC-MS: (ES, m/z): RT = 0.942 min, LCMS: m/z = 483 [M + 1], 1H NMR (400 MHz, DMSO-d6) δ 10.32 (s, 1H), 9.42 (d, J = 1.5 Hz, 1H), 8.39 (dd, J = 8.9, 7.8 Hz, 1H), 8.34 (s, 1H), 8.00 (d, J = 8.8 Hz, 1H), 6.90 (d, J = 2.3 Hz, 1H), 6.67 (d, J = 2.2 Hz, 1H), 5.00 (q, J = 6.7, 6.1 Hz, 1H), 3.78 (t, J = 4.9 Hz, 4H), 3.37 (dd, J = 6.1, 3.8 Hz, 4H), 2.82 (dd, J = 12.9, 8.2 Hz, 1H), 2.44 (dd, J = 12.9, 4.3 Hz, 1H), 2.16 (s, 6H), 1.45 (d, J = 5.9 Hz, 3H). |
| 184 | 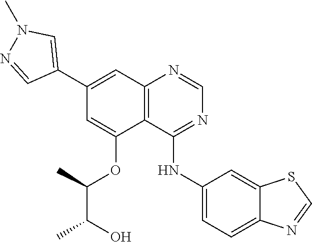 [SMILES: C[C@@H](O)[C@@H](C)Oc1cc(-c2cnn(C)c2)cc2ncnc(Nc3ccc4ncsc4c3)c12] | (2R,3R)-3-((4-(benzo[d]thiazol-6-ylamino)-7-(1-methyl-1H- pyrazol-4-yl)quinazolin-5-yl)oxy)butan-2-ol LC-MS: (ES, m/z): RT = 2.073 min, LCMS: m/z = 447 [M + 1]; ¹H NMR (DMSO) δ: 11.33 (s, 1H), 9.34 (s, 1H), 8.73 (s, 1H), 8.68 (s, 1H), 8.45 (s, 1H), 8.09 (d, J = 11.5 Hz, 2H), 7.91 (d, J = 8.8 Hz, 1H), 7.50 (s, 1H), 7.40 (s, 1H), 5.68 (s, 1H), 4.92 (d, J = 6.7 Hz, 1H), 3.93 (d, J = 6.9 Hz, 1H), 3.87 (s, 3H), 1.39 (d, J = 6.0 Hz, 3H), 1.22 (d, J = 6.3 Hz, 3H) |
| 185 | 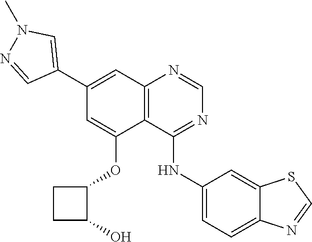 [SMILES: Cn1cc(-c2cc(O[C@H]3CC[C@H]3O)c3c(Nc4ccc5ncsc5c4)ncnc3c2)cn1] | (1R,2S)-2-((4-(benzo[d]thiazol-6-ylamino)-7-(1-methyl-1H- pyrazol-4-yl)quinazolin-5-yl)oxy)cyclobutan-1-ol LC-MS: (ES, m/z): RT = 2.121 min, LCMS: m/z = 445 [M + 1]; ¹H NMR (DMSO) δ: 11.47 (s, 1H), 9.41 (s, 1H), 8.80 (s, 1H), 8.74 (s, 1H), 8.50 (s, 1H), 8.19-8.12 (m, 2H), 7.98 (d, J = 8.8 Hz, 1H), 7.49 (s, 1H), 7.26 (s, 1H), 7.20 (s, 0H), 7.10 (s, 0H), 7.00 (s, 0H), 6.23 (s, 1H), 5.28 (d, J = 6.2 Hz, 1H), 4.67 (d, J = 5.8 Hz, 1H), 3.94 (s, 3H), 2.41 (s, 1H), 2.26 (d, J = 14.4 Hz, 1H), 2.18-2.07 (m, 2H) |
| 186 | 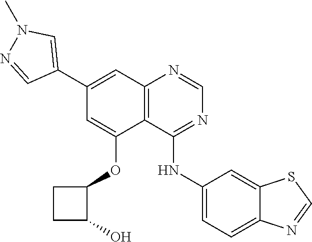 [SMILES: Cn1cc(-c2cc(O[C@@H]3CC[C@H]3O)c3c(Nc4ccc5ncsc5c4)ncnc3c2)cn1] | (1R,2R)-2-((4-(benzo[d]thiazol-6-ylamino)-7-(1-methyl-1H- pyrazol-4-yl)quinazolin-5-yl)oxy)cyclobutan-1-ol LC-MS: (ES, m/z): RT = 2.089 min, LCMS: m/z = 445 [M + 1], ¹H NMR (DMSO) δ: 10.62 (s, 1H), 9.34 (s, 1H), 8.70 (d, J = 5.7 Hz, 2H), 8.36 (s, 1H), 8.11 (d, J = 8.6 Hz, 1H), 8.02 (s, 1H), 7.77 (d, J = 8.7 Hz, 1H), 7.54 (s, 1H), 7.44 (s, 1H), 5.98 (s, 1H), 4.79 (q, J = 7.5 Hz, 1H), 4.35 (q, J = 7.9 Hz, 1H), 3.87 (s, 3H), 2.37 (d, J = 9.4 Hz, 1H), 2.15 (q, J = 9.6 Hz, 1H), 1.72 (q, J = 9.9 Hz, 1H), 1.51 (q, J = 9.7 Hz, 1H) |
| 187 | 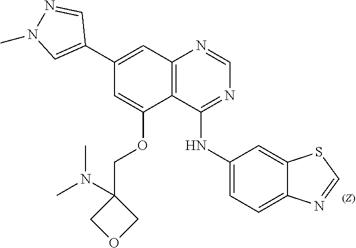 [SMILES: CN(C)C1(COc2cc(-c3cnn(C)c3)cc3ncnc(Nc4ccc5ncsc5c4)c23)COC1] | N-(5-((3-(dimethylamino)oxetan-3-yl)methoxy)-7-(1-methyl-1H- pyrazol-4-yl)quinazolin-4-yl)benzo[d]thiazol-6-amine LC-MS: (ES, m/z): RT = 2.153 min, LCMS: m/z = 488 [M + 1]; ¹H NMR (MeOD) δ: 9.27 (d, J = 2.1 Hz, 1H), 8.55 (s, 1H), 8.49 (s, 1H), 8.35 (s, 1H), 8.16 (s, 1H), 8.11 (d, J = 8.8 Hz, 1H), 7.76 (d, J = 8.8 Hz, 1H), 7.58 (s, 1H), 7.55 (s, 1H), 4.96 (d, J = 6.9 Hz, 2H), 4.81 (s, 2H), 4.71 (d, J = 6.9 Hz, 2H), 4.02 (d, J = 2.2 Hz, 3H), 2.48 (d, J = 2.2 Hz, 6H) |
| 188 | 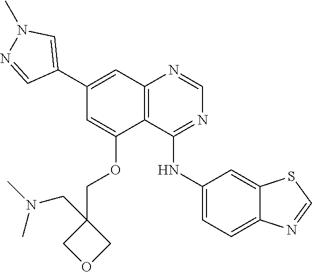 [SMILES: CN(C)CC1(COc2cc(-c3cnn(C)c3)cc3ncnc(Nc4ccc5ncsc5c4)c23)COC1] | N-(5-((3-((dimethylamino)methyl)oxetan-3-yl)methoxy)-7-(1- methyl-1H-pyrazol-4-yl)quinazolin-4-yl)benzo[d]thiazol-6-amine LC-MS: (ES, m/z): RT = 1.598 min, LCMS: m/z = 502 [M + 1]: ¹H NMR (MeOD) δ: 9.32 (s, 1H), 8.66 (s, 1H), 8.40 (s, 1H), 8.27 (d, J = 1.7 Hz, 1H), 8.15-8.07 (m, 2H), 7.71-7.63 (m, 2H), 7.58 (s, 1H), 4.96 (q, J = 6.8 Hz, 4H), 4.80 (s, 2H), 4.02 (s, 3H), 3.76 (s, 2H), 2.95 (s, 6H) |
| 189 | 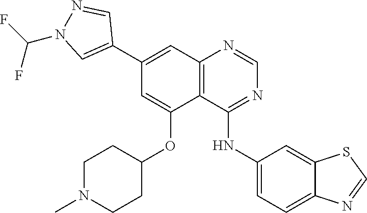 | N-(7-(1-(difluoromethyl)-1H-pyrazol-4-yl)-5-((1-methylpiperidin-4- yl)oxy)quinazolin-4-yl)benzo[d]thiazol-6-amine LC-MS: (ES, m/z): RT = 2.390 min, LCMS: m/z = 487 [M + 1]; ¹H NMR (500 MHz, DMSO-d6) δ 10.50-10.19 (m, 1H), 10.03-9.69 (m, 1H), 9.40 (s, 1H), 9.20-9.00 (m, 1H), 8.92-8.68 (m, 2H), 8.57 (s, 1H), 8.24-8.12 (m, 1H), 7.94 (t, J = 59.3 Hz, 1H), 7.81-7.68 (m, 2H), 7.68-7.49 (m, 1H), 5.36-5.09 (m, 1H), 3.75-3.43 (m, 2H), 3.31-3.13 (m, 3H), 2.98-2.78 (m, 3H), 2.71-2.57 (m, 1H), 2.35-2.00 (m, 2H). |
| 190 | 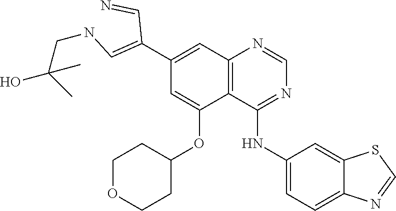 [SMILES: CC(C)(O)CN/C=C(\C=N)c1cc(OC2CCOCC2)c2c(Nc3ccc4ncsc4c3)ncnc2c1] | 1-(4-(4-(benzo[d]thiazol-6-ylamino)-5-((tetrahydro-2H-pyran-4- yl)oxy)quinazolin-7-yl)-1H-pyrazol-1-yl)-2-methylpropan-2-ol LC-MS: (ES, m/z): RT = 2.167 min, LCMS: m/z = 517 [M + 1]; ¹H NMR (CD3OD_SPE) δ: 9.29 (s, 1H), 8.76 (d, J = 6.2 Hz, 2H), 8.38 (s, 1H), 8.15 (d, J = 5.8 Hz, 2H), 7.79 (d, J = 8.8 Hz, 1H), 7.64 (s, 1H), 7.50 (s, 1H), 5.32 (s, 0H), 5.30 (dt, J = 10.7, 5.6 Hz, 1H), 4.18 (s, 2H), 4.07 (d, J = 11.8 Hz, 3H), 3.72 (t, J = 11.1 Hz, 2H), 3.30 (s, 7H), 2.38 (d, J = 12.8 Hz, 3H), 2.05 (d, J = 11.3 Hz, 3H), 1.23 (s, 6H) |
| 191 | 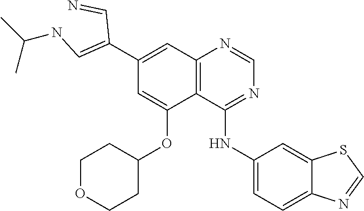 [SMILES: CC(C)N/C=C(\C=N)c1cc(OC2CCOCC2)c2c(Nc3ccc4ncsc4c3)ncnc2c1] | N-(7-(1-isopropyl-1H-pyrazol-4-yl)-5-((tetrahydro-2H-pyran-4- yl)oxy)quinazolin-4-yl)benzo[d]thiazol-6-amine LC-MS: (ES, m/z): RT = 2.390 min, LCMS: m/z = 487 [M + 1]; ¹H NMR (CD3OD_SPE) δ: 9.28 (d, J = 2.8 Hz, 1H), 8.79-8.72 (m, 2H), 8.48 (s, 1H), 8.15 (d, J = 8.3 Hz, 2H), 7.78 (d, J = 8.7 Hz, 1H), 7.64 (s, 1H), 7.49 (s, 1H), 5.29 (t, J = 7.7 Hz, 1H), 4.62 (dt, J = 14.8, 7.7 Hz, 1H), 4.07 (d, J = 11.7 Hz, 2H), 3.72 (t, J = 11.2 Hz, 2H), 3.30 (s, 5H), 2.38 (d, J = 12.6 Hz, 2H), 2.05 (q, J = 11.5 Hz, 3H), 1.60-1.54 (m, 7H) |
| 192 | 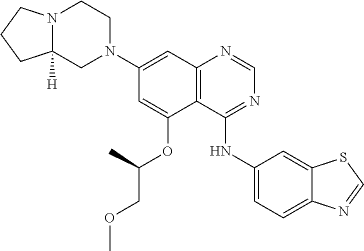 [SMILES: COC[C@@H](C)Oc1cc(N2CCN3CCC[C@@H]3C2)cc2ncnc(Nc3ccc4ncsc4c3)c12] | N-(7-((R)-hexahydropyrrolo[1,2-a]pyrazin-2(1H)-yl)-5-(((R)-1- methoxypropan-2-yl)oxy)quinazolin-4-yl)benzo[d]thiazol-6-amine LC-MS: (ES, m/z): RT = 1.81 min, LCMS: m/z = 491 [M + 1], ¹H NMR (500 MHz, Methanol-d₄) δ 9.18 (s, 1H), 8.58 (s, 1H), 8.49 (s, 1H), 8.09-8.00 (m, 1H), 7.76-7.65 (m, 1H), 6.86 (s, 1H), 6.54 (s, 1H), 5.11 (dt, J = 7.0, 3.7 Hz, 1H), 3.86-3.78 (m, 1H), 3.68-3.60 (m, 1H), 4.22-3.38 (m, 9H), 3.32 (s, 3H), 2.35-2.20 (m, 1H), 2.23- 1.96 (m, 2H), 1.95-1.73 (m, 1H), 1.54-1.41 (m, 3H). |
| 193 | 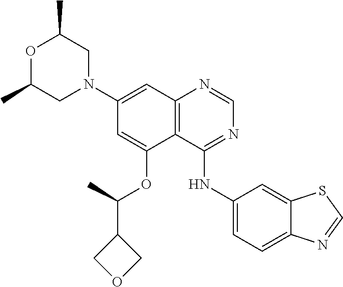 [SMILES: C[C@@H]1CN(c2cc(O[C@H](C)C3COC3)c3c(Nc4ccc5ncsc5c4)ncnc3c2)C[C@H](C)O1] | N-(7-((2S,6R)-2,6-dimethylmorpholino)-5-((R)-1-(oxetan-3- yl)ethoxy)quinazolin-4-yl)benzo[d]thiazol-6-amine LC-MS: (ES, m/z): RT = 1.807 min, LCMS: m/z = 493 [M + 1], ¹H NMR (MeOD) δ: 9.29 (s, 1H), 8.49 (s, 1H), 8.40 (s, 1H), 8.11 (d, J = 8.8 Hz, 1H), 7.71 (d, J = 9.0 Hz, 1H), 6.97 (s, 1H), 6.60 (s, 1H), 5.33 (t, J = 5.8 Hz, 1H), 5.02 (t, J = 7.4 Hz, 2H), 4.76 (t, J = 5.9 Hz, 1H), 4.63 (t, J = 5.7 Hz, 1H), 3.98 (t, J = 11.3 Hz, 2H), 3.80 (d, J = 9.7 Hz, 3H), 3.37 (s, 1H), 2.73 (t, J = 11.6 Hz, 2H), 1.49 (d, J = 6.1 Hz, 3H), 1.32 (d, J = 6.2 Hz, 6H) |
| 194 | 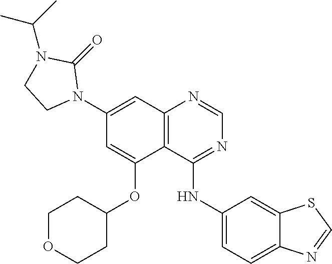 [SMILES: CC(C)N1CCN(c2cc(OC3CCOCC3)c3c(Nc4ccc5ncsc5c4)ncnc3c2)C1=O] | 1-(4-(benzo[d]thiazol-6-ylamino)-5-((tetrahydro-2H-pyran-4- yl)oxy)quinazolin-7-yl)-3-isopropylimidazolidin-2-one LC-MS: (ES, m/z): RT = 2.351 min, LCMS: m/z = 505 [M + 1], ¹H NMR (DMSO) δ: 10.55 (s, 1H), 9.34 (d, J = 2.8 Hz, 1H), 8.68 (s, 1H), 8.62 (s, 1H), 8.14-8.07 (m, 1H), 7.79 (s, 1H), 7.73 (d, J = 8.7 Hz, 1H), 7.36 (s, 1H), 4.98 (s, 1H), 4.05 (t, J = 7.8 Hz, 1H), 3.90 (q, J = 11.9, 10.1 Hz, 4H), 3.51 (t, J = 10.8 Hz, 2H), 3.45 (t, J = 8.1 Hz, 2H), 2.18 (d, J = 12.6 Hz, 2H), 1.93 (q, J = 12.0, 11.2 Hz, 2H), 1.10 (dd, J = 6.9, 2.8 Hz, 6H) |
| 195 | 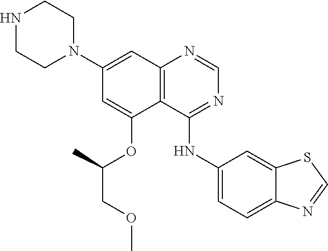 [SMILES: COC[C@@H](C)Oc1cc(N2CCNCC2)cc2ncnc(Nc3ccc4ncsc4c3)c12] | (R)-N-(5-((1-methoxypropan-2-yl)oxy)-7-(piperazin-1- yl)quinazolin-4-yl)benzo[d]thiazol-6-amine LC-MS: (ES, m/z): RT = 2.75 min, LCMS: m/z = 451 [M + 1]. ¹H NMR (500 MHz, Methanol-d₄) δ 9.30 (s, 1H), 8.70 (s, 1H), 8.64- 8.50 (m, 1H), 8.21-8.07 (m, 1H), 7.90-7.71 (m, 1H), 7.04 (s, 1H), 6.73 (s, 1H), 5.22 (s, 1H), 3.97-3.90 (m, 1H), 3.89-3.82 (m, 4H), 3.78-3.71 (m, 1H), 3.54-3.44 (m, 4H), 3.44 (s, 3H), 1.67-1.53 (m, 3H). |
| 196 | 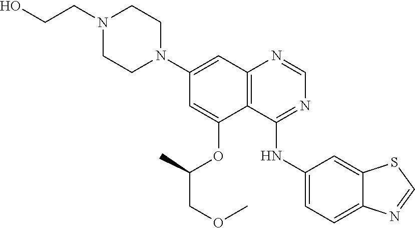 [SMILES: COC[C@@H](C)Oc1cc(N2CCN(CCO)CC2)cc2ncnc(Nc3ccc4ncsc4c3)c12] | (R)-2-(4-(4-(benzo[d]thiazol-6-ylamino)-5-((1-methoxypropan-2- yl)oxy)quinazolin-7-yl)piperazin-1-yl)ethan-1-ol LC-MS: (ES, m/z): RT = 1.77 min, LCMS: m/z = 495 [M + 1], ¹H NMR (500 MHz, Methanol-d₄) δ 9.30 (s, 1H), 8.70 (s, 1H), 8.62 (s, 1H), 8.14 (d, J = 8.8 Hz, 1H), 7.80 (d, J = 8.8 Hz, 1H), 7.04 (s, 1H), 6.75 (s, 1H), 5.21 (s, 1H), 4.12-3.79 (m, 4H), 3.79-3.72 (m, 1H), 3.72-3.49 (m, 4H), 3.49-3.38 (m, 6H), 1.65-1.52 (m, 3H). |
| 197 | 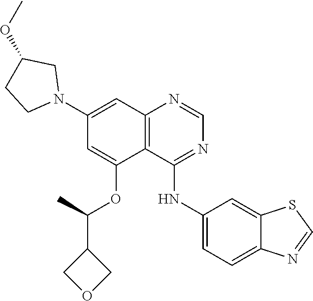 [SMILES: CO[C@H]1CCN(c2cc(O[C@H](C)C3COC3)c3c(Nc4ccc5ncsc5c4)ncnc3c2)C1] | N-(7-((S)-3-methoxypyrrolidin-1-yl)-5-((R)-1-(oxetan-3- yl)ethoxy)quinazolin-4-yl)benzo[d]thiazol-6-amine LC-MS: (ES, m/z): RT = 2.25 min, LCMS: m/z = 478 [M + 1], ¹H NMR (500 MHz, Methanol-d₄) δ 9.15 (s, 1H), 8.31 (s, 1H), 8.27 (s, 1H), 8.04-7.91 (m, 1H), 7.62-7.54 (m, 1H), 6.48 (s, 1H), 6.17 (s, 1H), 5.21-5.08 (m, 1H), 4.96-4.84 (m, 2H), 4.69-4.61 (m, 1H), 4.55-4.47 (m, 1H), 4.19-4.10 (m, 1H), 3.64-3.42 (m, 4H), 3.29- 3.23 (m, 2H), 3.21 (s, 3H), 2.28-2.16 (m, 1H), 2.15-2.02 (m, 1H), 1.46-1.35 (m, 3H). |
| 198 | 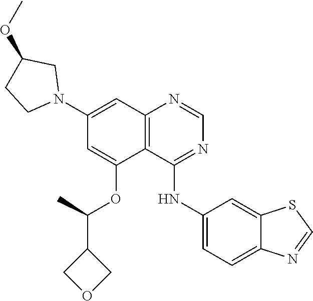 [SMILES: CO[C@@H]1CCN(c2cc(O[C@H](C)C3COC3)c3c(Nc4ccc5ncsc5c4)ncnc3c2)C1] | N-(7-((R)-3-methoxypyrrolidin-1-yl)-5-((R)-1-(oxetan-3- yl)ethoxy)quinazolin-4-yl)benzo[d]thiazol-6-amine LC-MS: (ES, m/z): RT = 2.25 min, LCMS: m/z = 478 [M + 1], ¹H NMR (500 MHz, Methanol-d4) δ 9.15 (s, 1H), 8.31 (s, 1H), 8.26 (s, 1H), 8.00-7.92 (m, 1H), 7.63-7.52 (m, 1H), 6.47 (s, 1H), 6.16 (s, 1H), 5.25-5.08 (m, 1H), 4.98-4.84 (m, 2H), 4.68-4.61 (m, 1H), 4.57-4.47 (m, 1H), 4.13 (s, 1H), 3.62-3.40 (m, 4H), 3.29-3.24 (m, 1H), 3.21 (s, 3H), 2.27-2.16 (m, 1H), 2.16-2.03 (m, 1H), 1.47- 1.32 (m, 3H). |
| 199 | 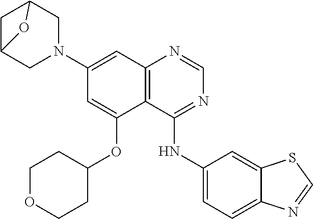 [SMILES: c1nc(Nc2ccc3ncsc3c2)c2c(OC3CCOCC3)cc(N3CC4CC(C3)O4)cc2n1] | N-(7-(6-oxa-3-azabicyclo[3.1.1]heptan-3-yl)-5-((tetrahydro-2H- pyran-4-yl)oxy)quinazolin-4-yl)benzo[d]thiazol-6-amine LC-MS: (ES, m/z): RT = 2.24 min, LCMS: m/z = 476 [M + 1], ¹H NMR (500 MHz, DMSO-d₆) δ 14.10 (s, 1H), 10.63 (s, 1H), 9.44 (s, 1H), 8.74 (s, 1H), 8.62 (s, 1H), 8.19 (d, J = 8.8 Hz, 1H), 7.80 (d, J = 8.8 Hz, 1H), 6.80 (s, 1H), 6.50 (s, 1H), 5.23 (s, 1H), 4.87-4.75 (m, 2H), 4.02-3.86 (m, 2H), 3.72 (t, J = 17.3 Hz, 3H), 3.64-3.52 (m, 2H), 3.28-3.16 (m, 1H), 2.31-2.12 (m, 2H), 2.07-1.80 (m, 3H). |
| 200 | 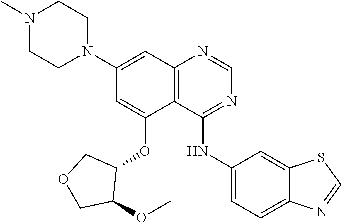 [SMILES: CO[C@H]1COC[C@@H]1Oc1cc(N2CCN(C)CC2)cc2ncnc(Nc3ccc4ncsc4c3)c12] | (3S,4S)-4-((4-(benzo[d]thiazol-6-ylamino)-7-(4-methylpiperazin-1- yl)quinazolin-5-yl)oxy)tetrahydrofuran-3-ol LC-MS: (ES, m/z): RT = 1.67 min, LCMS: m/z = 494 [M + 1], ¹H NMR (500 MHz, Methanol-d4) δ 9.29 (s, 1H), 8.70 (s, 1H), 8.62 (s, 1H), 8.13 (d, J = 8.8 Hz, 1H), 7.80 (d, J = 8.8 Hz, 1H), 7.03 (s, 1H), 6.74 (s, 1H), 5.27-5.16 (m, 1H), 3.97-3.91 (m, 1H), 3.86-3.79 (m, 2H), 3.78-3.71 (m, 1H), 3.69-3.52 (m, 8H), 3.52-3.48 (m, 2H), 3.47 (s, 3H), 3.45 (s, 3H), 1.60 (d, J = 6.3 Hz, 3H) |
| 201 | 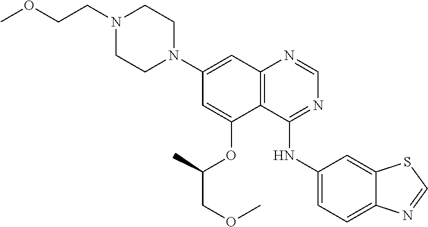 [SMILES: COCCN1CCN(c2cc(O[C@H](C)COC)c3c(Nc4ccc5ncsc5c4)ncnc3c2)CC1] | (R)-N-(7-(4-(2-methoxyethyl)piperazin-1-yl)-5-((1-methoxypropan- 2-yl)oxy)quinazolin-4-yl)benzo[d]thiazol-6-amine LC-MS: (ES, m/z): RT = 1.78 min, LCMS: m/z = 510 [M + 1], ¹H NMR (500 MHz, Methanol-d4) δ 9.29 (s, 1H), 8.70 (s, 1H), 8.62 (s, 1H), 8.13 (d, J = 8.8 Hz, 1H), 7.80 (d, J = 8.8 Hz, 1H), 7.03 (s, 1H), 6.74 (s, 1H), 5.27-5.16 (m, 1H), 3.97-3.91 (m, 1H), 3.86-3.79 (m, 2H), 3.78-3.71 (m, 1H), 3.69-3.52 (m, 8H), 3.52-3.48 (m, 2H), 3.47 (s, 3H), 3.45 (s, 3H), 1.60 (d, J = 6.3 Hz, 3H). |
| 202 | 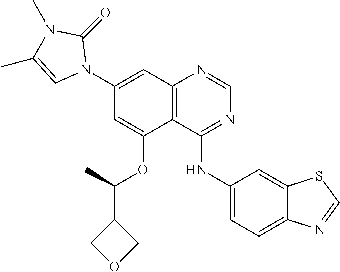 [SMILES: Cc1cn(-c2cc(O[C@H](C)C3COC3)c3c(Nc4ccc5ncsc5c4)ncnc3c2)c(=O)n1C] | (R)-1-(4-(benzo[d]thiazol-6-ylamino)-5-(1-(oxetan-3- yl)ethoxy)quinazolin-7-yl)-3,4-dimethyl-1,3-dihydro-2H-imidazol-2- one LC-MS: (ES, m/z): RT = 2.026 min, LCMS: m/z = 489 [M + 1], 1H NMR (DMSO) δ: 10.00 (s, 1H), 9.25 (d, J = 3.1 Hz, 1H), 8.65 (d, J = 3.2 Hz, 1H), 8.45 (d, J = 3.1 Hz, 1H), 8.01 (dd, J = 8.8, 3.0 Hz, 1H), 7.80 (s, 1H), 7.70 (d, J = 9.0 Hz, 1H), 7.65 (d, J = 3.1 Hz, 1H), 7.09 (s, 1H), 5.16 (h, J = 5.8 Hz, 1H), 4.76 (dq, J = 11.1, 6.5 Hz, 2H), 4.58 (p, J = 4.6 Hz, 1H), 4.49 (q, J = 5.5, 5.1 Hz, 1H), 3.48-3.42 (m, 1H), 3.11 (d, J = 3.2 Hz, 3H), 1.55 (d, J = 3.3 Hz, 3H), 1.39- 1.34 (m, 3H) |
| 203 | 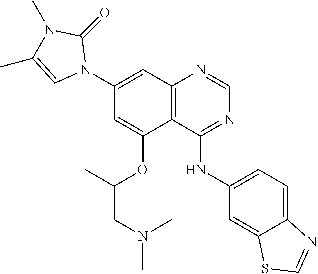 [SMILES: Cc1cn(-c2cc(OC(C)CN(C)C)c3c(Nc4ccc5ncsc5c4)ncnc3c2)c(=O)n1C] | 1-(4-(benzo[d]thiazol-6-ylamino)-5-((1-(dimethylamino)propan-2- yl)oxy)quinazolin-7-yl)-3,4-dimethyl-1,3-dihydro-2H-imidazol-2- one LC-MS: (ES, m/z): RT = 1.620 min, LCMS: m/z = 490 [M + 1], ¹H NMR (DMSO) δ: 10.52 (d, J = 3.5 Hz, 1H), 9.25 (t, J = 2.5 Hz, 1H), 8.71 (d, J = 3.4 Hz, 1H), 8.46 (d, J = 3.4 Hz, 1H), 8.03 (dd, J = 9.3, 3.4 Hz, 1H), 7.80 (d, J = 3.5 Hz, 1H), 7.75-7.70 (m, 1H), 7.58 (d, J = 3.4 Hz, 1H), 7.07 (s, 1H), 4.88 (dt, J = 10.3, 5.0 Hz, 1H), 2.95 (t, J = 10.0 Hz, 1H), 2.40 (d, J = 12.0 Hz, 1H), 2.16 (d, J = 3.4 Hz, 6H), 2.05 (d, J = 3.4 Hz, 3H), 1.65 (s, 3H), 1.48-1.43 (m, 3H) |
| 204 | 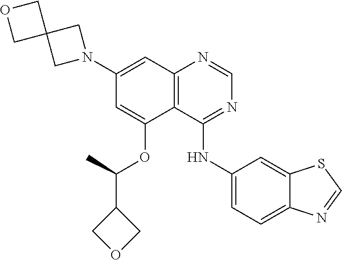 [SMILES: C[C@@H](Oc1cc(N2CC3(COC3)C2)cc2ncnc(Nc3ccc4ncsc4c3)c12)C1COC1] | (R)-N-(5-(1-(oxetan-3-yl)ethoxy)-7-(2-oxa-6-azaspiro[3.3]heptan-6- yl)quinazolin-4-yl)benzo[d]thiazol-6-amine LC-MS: (ES, m/z): RT = 2.084 min, LCMS: m/z = 476 [M + 1], ¹H NMR (DMSO) δ: 9.86 (s, 1H), 9.22 (s, 1H), 8.64 (s, 1H), 8.28 (s, 1H), 7.98 (d, J = 8.7 Hz, 1H), 7.66 (d, J = 8.8 Hz, 1H), 6.29 (s, 1H), 6.11 (s, 1H), 5.16 (s, 1H), 4.74 (d, J = 8.3 Hz, 2H), 4.69 (s, 4H), 4.47 (s, 1H), 4.45 (s, 1H), 4.13 (d, J = 3.3 Hz, 4H), 3.42 (s, 1H), 1.30 (t, J = 4.7 Hz, 3H) |
| 205 206 | 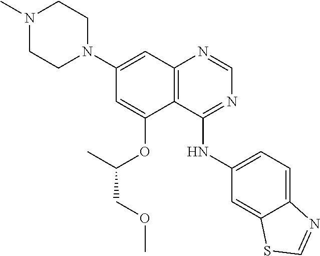 [SMILES: COC[C@H](C)Oc1cc(N2CCN(C)CC2)cc2ncnc(Nc3ccc4ncsc4c3)c12] or 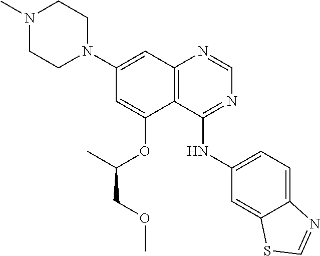 [SMILES: COC[C@@H](C)Oc1cc(N2CCN(C)CC2)cc2ncnc(Nc3ccc4ncsc4c3)c12] | (S)-N-(5-((1-methoxypropan-2-yl)oxy)-7-(4-methylpiperazin-1- yl)quinazolin-4-yl)benzo[d]thiazol-6-amine OR (R)-N-(5-((1- methoxypropan-2-yl)oxy)-7-(4-methylpiperazin-1-yl)quinazolin-4- yl)benzo[d]thiazol-6-amine First Eluting Isomer: LC-MS: (ES, m/z): RT = 1.066 min, LCMS: m/z = 465 [M + 1], Chiral-HPLC (ES): RT = 3.228 min; ¹H NMR (400 MHz, DMSO-d6) δ 10.41 (s, 1H), 9.31 (s, 1H), 8.89 (d, J = 2.1 Hz, 1H), 8.49 (s, 1H), 8.11 (d, J = 8.8 Hz, 1H), 7.75 (dd, J = 8.9, 2.1 Hz, 1H), 6.76 (d, J = 2.2 Hz, 1H), 6.97 (d, J = 2.3 Hz, 1H), 5.17 (dd, J = 6.6, 3.5 Hz, 1H), 3.83 (dd, J = 10.7, 2.9 Hz, 1H), 3.72 (dd, J = 10.7, 4.2 Hz, 1H), 3.63-3.43 (m, 4H), 3.38-3.06 (m, 7H), 2.83 (s, 3H), 1.46 (d, J = 6.2 Hz, 3H). Second Eluting Isomer: LC-MS: (ES, m/z): RT = 1.008 min, LCMS: m/z = 465 [M + 1], Chiral-HPLC (ES): RT = 3.949 min; ¹H NMR (400 MHz, DMSO-d6) δ 10.42 (s, 1H), 9.31 (s, 1H), 8.89 (d, J = 2.1 Hz, 1H), 8.50 (s, 1H), 8.12 (d, J = 8.8 Hz, 1H), 7.75 (dd, J = 8.9, 2.2 Hz, 1H), 6.97 (d, J = 2.2 Hz, 1H), 6.76 (d, J = 2.1 Hz, 1H), 5.18 (d, J = 6.8 Hz, 1H), 3.83 (dd, J = 10.7, 2.8 Hz, 1H), 3.74 (d, J = 4.2 Hz, 1H), 3.63-3.43 (s, 8H) ,3.33-3.23 (m, 3H), 2.85 (s, 3H), 1.46 (d, J = 6.2 Hz, 3H). |
| 207 208 | 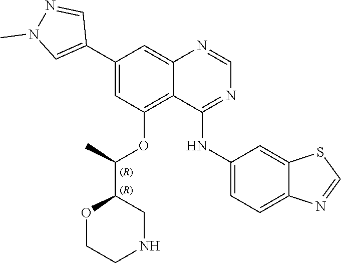 or 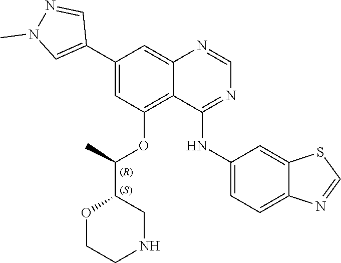 | N-(7-(1-methyl-1H-pyrazol-4-yl)-5-((R)-1-((S)-morpholin-2- yl)ethoxy)quinazolin-4-yl)benzo[d]thiazol-6-amine OR N-(7-(1- methyl-1H-pyrazol-4-yl)-5-((R)-1-((R)-morpholin-2- yl)ethoxy)quinazolin-4-yl)benzo[d]thiazol-6-amine First Eluting Isomer: LC-MS: (ES, m/z): RT = 1.236 min, LCMS: m/z = 488 [M + 1], Chiral-HPLC (ES): RT = 5.442; ¹H NMR (400 MHz, DMSO-d6) δ 10.63 (s, 1H), 9.31 (s, 1H), 8.95 (d, J = 2.1 Hz, 1H), 8.56 (s, 1H), 8.46 (s, 1H), 8.17-8.10 (m, 2H), 7.82 (dd, J = 8.9, 2.1 Hz, 1H), 7.56 (d, J = 1.4 Hz, 1H), 7.39 (d, J = 1.6 Hz, 1H), 5.05 (dd, J = 6.4, 2.8 Hz, 1H), 3.91 (m, 4H), 3.75-3.67 (m, 1H), 3.61 (td, J = 11.3, 2.6 Hz, 1H), 2.85 (d, J = 11.7 Hz, 1H), 2.75-2.60 (m, 3H), 1.49 (d, J = 6.2 Hz, 3H). Second Eluting Isomer: LC-MS: (ES, m/z): RT = 1.008 min, LCMS: m/z = 488 [M + 1], Chiral-HPLC (ES): RT = 6.459; ¹H NMR (400 MHz, DMSO-d6) δ 10.57 (s, 1H), 9.31 (s, 1H), 8.92 (d, J = 2.1 Hz, 1H), 8.56 (s, 1H), 8.46 (s, 1H), 8.25 (s, 1H), 8.17-8.09 (m, 2H), 7.75 (dd, J = 8.9, 2.2 Hz, 1H), 7.58 (d, J = 1.5 Hz, 1H), 7.47 (d, J = 1.6 Hz, 1H), 5.14 (dd, J = 6.3, 2.8 Hz, 1H), 3.89 (td, J = 11.7, 10.3, 2.1 Hz, 4H), 3.61-3.50 (m, 1H), 2.93 (dd, J = 12.1, 2.3 Hz, 1H), 2.76-2.68 (m, 2H), 2.60 (dd, J = 12.2, 10.5 Hz, 1H), 1.43 (d, J = 6.2 Hz, 3H). |
| 209 210 | 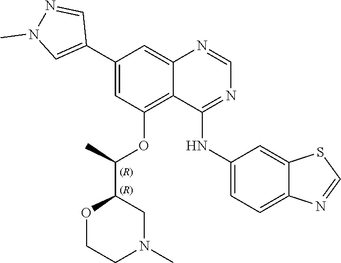 or 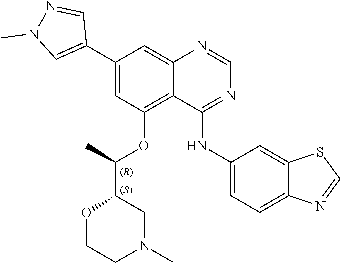 | N-(7-(1-methyl-1H-pyrazol-4-yl)-5-((R)-1-((S)-4-methylmorpholin- 2-yl)ethoxy)quinazolin-4-yl)benzo[d]thiazol-6-amine OR N-(7-(1- methyl-1H-pyrazol-4-yl)-5-((R)-1-((R)-4-methylmorpholin-2- yl)ethoxy)quinazolin-4-yl)benzo[d]thiazol-6-amine First Eluting Isomer: LC-MS: (ES, m/z): RT = 1.479 min, LCMS: m/z = 502 [M + 1], Chiral-HPLC (ES): RT = 3.463 min; ¹H NMR (400 MHz, DMSO-d6) δ 10.52 (s, 1H), 9.31 (s, 1H), 8.92 (d, J = 2.1 Hz, 1H), 8.56 (s, 1H), 8.45 (s, 1H), 8.17-8.09 (m, 2H), 7.75 (dd, J = 8.9, 2.2 Hz, 1H), 7.58 (d, J = 1.4 Hz, 1H), 7.47 (d, J = 1.6 Hz, 1H), 5.19 (dd, J = 6.3, 2.9 Hz, 1H), 4.03-3.93 (m, 1H), 3.91 (s, 4H), 3.68- 3.58 (m, 1H), 2.86 (d, J = 11.0 Hz, 1H), 2.70-2.62 (m, 1H), 2.21 (s, 3H), 2.04 (td, J = 11.4, 3.2 Hz, 1H), 1.94 (t, J = 10.8 Hz, 1H), 1.44 (d, J = 6.2 Hz, 3H). Second Eluting Isomer: LC-MS: (ES, m/z): RT = 1.066 min, LCMS: m/z = 502 [M + 1], Chiral-HPLC (ES): RT = 4.515 min; ¹H NMR (400 MHz, DMSO-d6) δ 10.58 (s, 1H), 9.31 (s, 1H), 8.95 (d, J = 2.1 Hz, 1H), 8.57 (s, 1H), 8.46 (s, 1H), 8.14 (d, J = 8.5 Hz, 2H), 7.80 (dd, J = 8.8, 2.2 Hz, 1H), 7.56 (s, 1H), 7.40 (s, 1H), 5.14-5.07 (m, 1H), 3.97 (d, J = 11.4 Hz, 1H), 3.91 (s, 3H), 3.82 (d, J = 10.2 Hz, 1H), 3.74-3.63 (m, 1H), 2.80 (d, J = 11.1 Hz, 1H), 2.60 (d, J = 11.5 Hz, 1H), 2.12 (s, 3H), 2.06 (t, J = 10.7 Hz, 1H), 1.85 (t, J = 10.6 Hz, 1H), 1.50 (d, J = 6.2 Hz, 3H). |
| 211 212 | 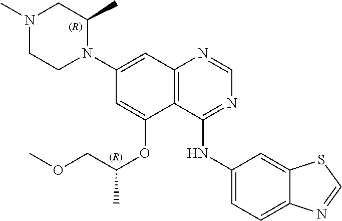 or 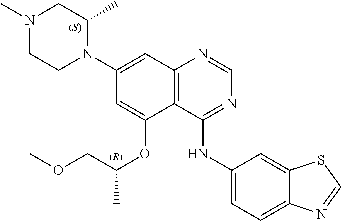 | N-(7-((S)-2,4-dimethylpiperazin-1-yl)-5-(((R)-1-methoxypropan-2- yl)oxy)quinazolin-4-yl)benzo[d]thiazol-6-amine OR N-(7-((R)-2,4- dimethylpiperazin-1-yl)-5-(((R)-1-methoxypropan-2- yl)oxy)quinazolin-4-yl)benzo[d]thiazol-6-amine First Eluting Isomer: LC-MS: (ES, m/z): RT = 1.150 min, LCMS: m/z = 479 [M + 1], Chiral-HPLC (ES): RT = 2.982 min; ¹H NMR (400 MHz, DMSO-d6) δ 10.33 (s, 1H), 9.28 (s, 1H), 8.91 (d, J = 2.1 Hz, 1H), 8.42 (s, 1H), 8.09 (d, J = 8.8 Hz, 1H), 7.74 (dd, J = 8.9, 2.2 Hz, 1H), 6.87 (d, J = 2.3 Hz, 1H), 6.60 (d, J = 2.2 Hz, 1H), 5.19- 5.11 (m, 1H), 4.35 (s, 1H), 3.82 (dd, J = 10.7, 2.9 Hz, 1H), 3.70 (dd, J = 10.7, 4.1 Hz, 1H), 3.57 (m, 1H), 3.26-3.10 (m, 1H), 2.99-2.90 (m, 1H), 2.90-2.79 (m, 1H), 2.27 (s, 3H), 2.20-2.10 (m, 1H), 1.45 (d, J = 6.2 Hz, 3H), 1.15 (d, J = 6.6 Hz, 3H). Second Eluting Isomer: LC-MS: (ES, m/z): RT = 1.451 min, LCMS: m/z = 479 [M + 1], Chiral-HPLC (ES): RT = 3.535 min; ¹H NMR (400 MHz, DMSO-d6) δ 10.76 (s, 1H), 9.40 (d, J = 8.4 Hz, 1H), 8.71 (d, J = 2.2 Hz, 1H), 8.66 (s, 1H), 8.21-8.10 (m, 1H), 7.79- 7.68 (m, 1H), 7.00 (d, J = 2.4 Hz, 1H), 6.67 (d, J = 2.2 Hz, 1H), 5.23 (dt, J = 7.0, 3.8 Hz, 1H), 4.68 (s, 1H), 4.10-4.00 (m, 1H), 3.82 (dd, J = 10.7, 2.9 Hz, 1H), 3.70 (dd, J = 10.7, 4.1 Hz, 1H), 3.57- 3.33 (m, 5H), 3.26-3.10 (m, 1H), 2.88 (s, 3H), 1.46 (d, J = 6.2 Hz, 3H), 1.25 (d, J = 7.0 Hz, 3H). |
| 226 | 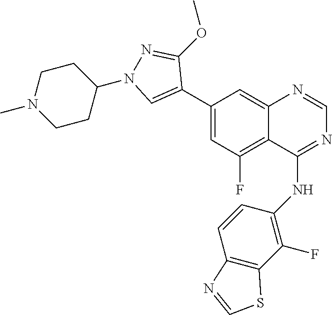 [SMILES: COc1nn(C2CCN(C)CC2)cc1-c1cc(F)c2c(Nc3ccc4ncsc4c3F)ncnc2c1] | 7-fluoro-N-(5-fluoro-7-(3-methoxy-1-(1-methylpiperidin-4-yl)-1H- pyrazol-4-yl)quinazolin-4-yl)benzo[d]thiazol-6-amine LC-MS: (ES, m/z): RT = 1.814 min, LC-MS: m/z = 508.2 [M + 1]. ¹H NMR (400 MHz, Chloroform-d) δ 9.48 (s, 1H), 9.27 (d, 1H, J = 12.8 Hz), 8.47 (d, 2H, J = 16.8 Hz), 8.02 (d, 1H, J = 8.4 Hz), 7.94 (s, 1H), 7.87 (t, 1H, J = 8.0 Hz), 7.77 (d, 1H, J = 14.8 Hz), 4.00 (s, 3H), 2.89 (d, 2H, J = 9.6 Hz), 2.24 (s, 3H), 2.06 (d, 4H, J = 11.6 Hz), 2.00-1.94 (m, 3H). |
| 227 | 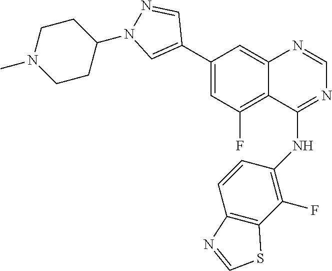 [SMILES: CN1CCC(n2cc(-c3cc(F)c4c(Nc5ccc6ncsc6c5F)ncnc4c3)cn2)CC1] | 7-fluoro-N-(5-fluoro-7-(1-(1-methylpiperidin-4-yl)-1H-pyrazol-4- yl)quinazolin-4-yl)benzo[d]thiazol-6-amine LC-MS: (ES, m/z): RT = 2.300 min, LC-MS: m/z = 478.1 [M + 1]. ¹H NMR (400 MHz, DMSO-d₆) δ 9.46 (s, 1H), 9.28 (s, 1H), 8.60 (s, 1H), 8.45 (s, 1H), 8.19 (s, 1H), 8.01 (d, 1H, J = 8.8 Hz), 7.87-7.82 (m, 2H), 7.79 (d, 1H, J = 13.2 Hz), 4.17-4.12 (m, 1H), 2.87 (d, 2H, J = 10.8 Hz), 2.22 (s, 3H), 2.10-1.98 (m, 6H). |
| 240 | 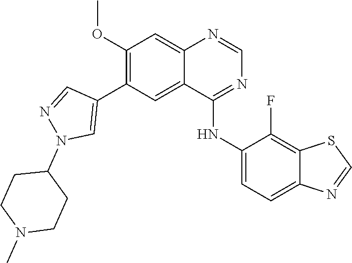 [SMILES: COc1cc2ncnc(Nc3ccc4ncsc4c3F)c2cc1-c1cnn(C2CCN(C)CC2)c1] | 7-fluoro-N-(7-methoxy-6-(1-(1-methylpiperidin-4-yl)-1H-pyrazol-4- yl)quinazolin-4-yl)benzo[d]thiazol-6-amine LC-MS: (ES, m/z): RT = 1.202 min, LCMS: m/z = 490 [M + 1]. ¹H- NMR 1H NMR (400 MHz, DMSO-d6) δ 9.88 (s, 1H), 9.48 (d, J = 1.5 Hz, 1H), 8.70 (s, 1H), 8.39 (s, 1H), 8.29 (s, 1H), 8.11 (s, 1H), 8.03 (d, J = 8.6 Hz, 1H), 7.74 (dd, J = 8.7, 7.5 Hz, 1H), 7.28 (s, 1H), 4.25-4.15 (m, 1H), 4.05 (s, 3H), 2.89 (d, J = 8.2 Hz, 2H), 2.22 (d, J = 1.4 Hz, 3H), 2.13-1.97 (m, 6H). |
| 241 | 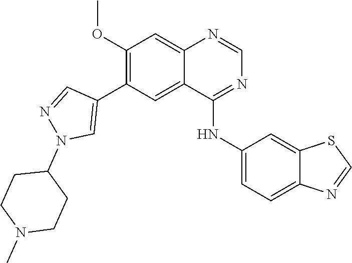 [SMILES: COc1cc2ncnc(Nc3ccc4ncsc4c3)c2cc1-c1cnn(C2CCN(C)CC2)c1] | N-(7-methoxy-6-(1-(1-methylpiperidin-4-yl)-1H-pyrazol-4- yl)quinazolin-4-yl)benzo[d]thiazol-6-amine LC-MS: (ES, m/z): RT = 1.222 min, LCMS: m/z = 472 [M + 1]. ¹H- NMR 1H NMR (300 MHz, DMSO-d6) δ 9.83 (s, 1H), 9.30 (s, 1H), 8.81-8.65 (m, 2H), 8.53 (s, 1H), 8.28 (s, 1H), 8.18-8.04 (m, 2H), 7.90 (dd, J = 8.9, 2.2 Hz, 1H), 7.26 (s, 1H), 4.19 (s, 1H), 4.03 (s, 3H), 2.87 (d, J = 5.8 Hz, 2H), 2.21 (s, 3H), 2.10-1.94 (m, 6H). |
| 242 | 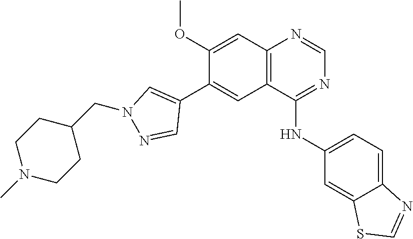 [SMILES: COc1cc2ncnc(Nc3ccc4ncsc4c3)c2cc1-c1cnn(CC2CCN(C)CC2)c1] | N-(7-methoxy-6-(1-((1-methylpiperidin-4-yl)methyl)-1H-pyrazol-4- yl)quinazolin-4-yl)benzo[d]thiazol-6-amine LC-MS: (ES, m/z): RT = 2.173 min, LCMS: m/z = 486 [M + 1], ¹H NMR (300 MHz, DMSO-d6) δ 9.87 (s, 1H), 9.32 (s, 1H), 8.81-8.71 (m, 2H), 8.55 (s, 1H), 8.26 (s, 1H), 8.16 (s, 1H), 8.12 (d, J = 8.9 Hz, 1H), 7.92 (dt, J = 8.9, 2.6 Hz, 1H), 7.29 (s, 1H), 4.12-4.03 (m, 5H), 2.75 (d, J = 11.1 Hz, 2H), 2.14 (s, 3H), 1.81 (t, J = 11.2 Hz, 3H), 1.49 (d, J = 12.6 Hz, 2H), 1.36-1.18 (m, 2H). |
| 243 | 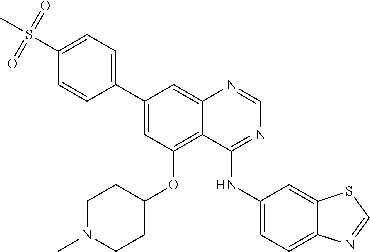 [SMILES: CN1CCC(Oc2cc(-c3ccc(S(C)(=O)=O)cc3)cc3ncnc(Nc4ccc5ncsc5c4)c23)CC1] | N-(5-((1-methylpiperidin-4-yl)oxy)-7-(4- (methylsulfonyl)phenyl)quinazolin-4-yl)benzo[d]thiazol-6-amine LC-MS: (ES, m/z): RT = 1.796 min, LCMS: m/z = 546 [M + 1], ¹H NMR (500 MHz, Chloroform-d) δ 10.85 (s, 1H), 9.12 (s, 1H), 8.66 (s, 1H), 8.62 (s, 1H), 8.31-8.23 (m, 1H), 8.01-7.89 (m, 2H), 7.89- 7.53 (m, 5H), 7.33 (s, 1H), 5.46-5.28 (m, 1H), 3.80-3.62 (m, 1H), 3.43-3.23 (m, 2H), 3.09-3.02 (m, 2H), 3.00-2.93 (m, 2H), 2.93-2.86 (m, 2H), 2.71 (s, 3H), 1.25 (s, 3H). |
| 245 | 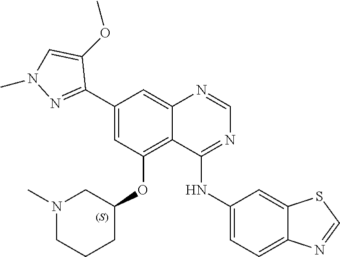 | (S)-N-(7-(4-methoxy-1-methyl-1H-pyrazol-3-yl)-5-((1- methylpiperidin-3-yl)oxy)quinazolin-4-yl)benzo[d]thiazol-6-amine LC-MS: (ES, m/z): RT = 1.803 min, LCMS: m/z = 502 [M + 1], ¹H NMR (300 MHz, DMSO-d6) δ 10.91 (s, 1H), 9.31 (s, 1H), 8.93 (d, J = 2.0 Hz, 1H), 8.58 (s, 1H), 8.12 (d, J = 8.9 Hz, 1H), 8.01 (dd, J = 8.9, 2.1 Hz, 1H), 7.88 (d, J = 1.3 Hz, 1H), 7.71 (s, 1H), 7.59 (d, J = 1.5 Hz, 1H), 5.09 (s, 1H), 3.87 (d, J = 7.4 Hz, 6H), 3.17 (d, J = 12.1 Hz, 1H), 2.86 (d, J = 11.4 Hz, 1H), 2.43 (d, J = 12.0 Hz, 1H), 2.32 (s, 3H), 2.11 (d, J = 11.6 Hz, 2H), 1.83-1.47 (m, 3H). |
| 246 | 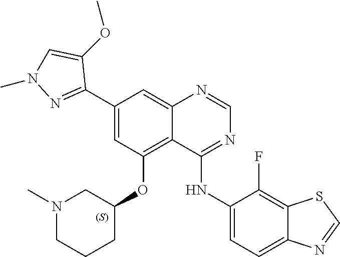 | (S)-7-fluoro-N-(7-(4-methoxy-1-methyl-1H-pyrazol-3-yl)-5-((1- methylpiperidin-3-yl)oxy)quinazolin-4-yl)benzo[d]thiazol-6-amine LC-MS: (ES, m/z): RT = 1.756 min, LCMS: m/z = 520 [M + 1], ¹H NMR (300 MHz, DMSO-d6) δ 10.67 (s, 1H), 9.49-9.41 (m, 1H), 8.43 (s, 1H), 8.07-7.85 (m, 3H), 7.71 (s, 1H), 7.59 (s, 1H), 5.03 (s, 1H), 3.87 (d, J = 8.1 Hz, 6H), 2.94 (d, J = 11.6 Hz, 1H), 2.57 (d, J = 11.4 Hz, 1H), 2.44 (d, J = 11.2 Hz, 1H), 2.10 (d, J = 13.1 Hz, 5H), 1.84-1.66 (m, 2H), 1.53 (s, 1H). |
| 247 | 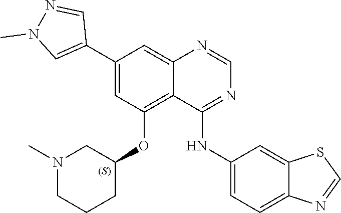 | (S)-N-(7-(1-methyl-1H-1,2,3-triazol-4-yl)-5-((1-methylpiperidin-3- yl)oxy)quinazolin-4-yl)benzo[d]thiazol-6-amine LC-MS: (ES, m/z): RT = 1.604 min, LCMS: m/z = 473 [M + 1], ¹H NMR (300 MHz, DMSO-d6) δ 10.96 (s, 1H), 9.33 (s, 1H), 9.08- 8.77 (m, 2H), 8.62 (s, 1H), 8.16-7.96 (m, 2H), 7.83 (d, J = 1.4 Hz, 1H), 7.67 (s, 1H), 5.26 (s, 1H), 4.16 (s, 3H), 3.22 (s, 1H) 2.90 (s, 1H), 2.46 (s, 1H), 2.34 (s, 3H), 2.14 (d, J = 12.1 Hz, 2H), 1.58 (s, 3H), 1.25 (s, 1H). |
| 248 | 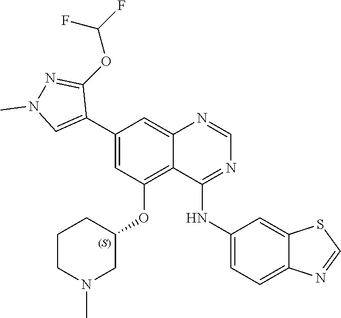 | (S)-N-(7-(3-(difluoromethoxy)-1-methyl-1H-pyrazol-4-yl)-5-((1- methylpiperidin-3-yl)oxy)quinazolin-4-yl)benzo[d]thiazol-6-amine LC-MS: (ES, m/z): RT = 1.930 min, LCMS: m/z = 538 [M + 1],¹H NMR (400 MHz, DMSO-d6) 1H δ 10.87 (s, 1H), 9.30 (s, 1H), 8.92 (d, J = 2.1 Hz, 1H), 8.57 (s, 1H), 8.47 (s, 1H), 8.11 (d, J = 8.8 Hz, 1H), 7.99 (dd, J = 8.9, 2.1 Hz, 1H), 7.65-7.20 (m, 3H), 5.15 (s, 1H), 3.83 (s, 3H), 3.18 (d, J = 12.1 Hz, 1H), 2.85 (d, J = 11.0 Hz, 1H), 2.43 (d, J = 12.1 Hz, 1H), 2.32 (s, 3H), 2.11 (dd, J = 11.4, 8.5 Hz, 2H), 1.74 (t, J = 12.2 Hz, 1H), 1.66-1.44 (m, 2H). |
| 249 | 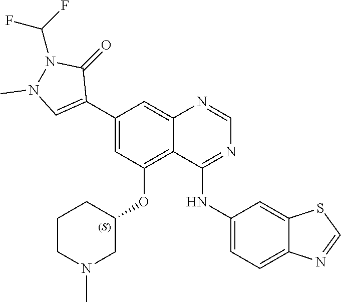 | (S)-4-(4-(benzo[d]thiazol-6-ylamino)-5-((1-methylpiperidin-3- yl)oxy)quinazolin-7-yl)-2-(difluoromethyl)-1-methyl-1,2-dihydro- 3H-pyrazol-3-one LC-MS: (ES, m/z): RT = 1.616 min, LCMS: m/z = 538 [M + 1], ¹H NMR (400 MHz, DMSO-d6) 1H δ 10.86 (s, 1H), 9.30 (s, 1H), 8.92 (d, J = 2.4 Hz, 2H), 8.56 (s, 1H), 8.10 (d, J = 8.8 Hz, 1H), 7.99 (dd, J = 8.9, 2.1 Hz, 1H), 7.91 (d, J = 1.4 Hz, 1H), 7.80-7.49 (m, 2H), 5.12-5.08 (m, 1H), 3.65 (s, 3H), 3.18 (d, J = 12.1 Hz, 1H), 2.85 (d, J = 11.1 Hz, 1H), 2.43 (d, J = 12.0 Hz, 1H), 2.32 (s, 3H), 2.17-2.06 (m, 2H), 1.76 (d, J = 12.5 Hz, 1H), 1.68-1.50 (m, 2H). |
| 250 | 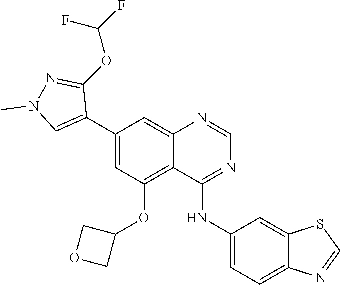 [SMILES: Cn1cc(-c2cc(OC3COC3)c3c(Nc4ccc5ncsc5c4)ncnc3c2)c(OC(F)F)n1] | N-(7-(3-(difluoromethoxy)-1-methyl-1H-pyrazol-4-yl)-5-(oxetan-3- yloxy)quinazolin-4-yl)benzo[d]thiazol-6-amine LC-MS: (ES, m/z): RT = 1.319 min, LCMS: m/z = 497.05 [M + 1], ¹H NMR (300 MHz, DMSO-d6) δ 9.43 (s, 1H), 8.43 (s, 1H), 8.18-8.14 (m, 3H), 7.70-7.46 (m, 3H), 7.35 (d, J = 1.5 Hz, 1H), 5.16 (s, 1H), 4.69 (s, 1H), 4.17 (d, J = 3.9 Hz, 2H), 3.82 (s, 3H), 3.68-3.64 (m, 2H). LC-MS: (ES, m/z): RT = 0.990 min, LCMS: m/z = 489[M + 1], 1H NMR (300 MHz, DMSO-d6) δ 9.58 (s, 1H), 9.33 (s, 1H), 8.74 (d, J = 2.0 Hz, 1H), 8.38 (d, J = 17.3 Hz, 2H), 8.17-8.06 (m, 2H), 7.90 (dd, J = 7.5, 2.3 Hz, 2H), 7.49 (s, 1H), 7.06 (d, J = 2.4 Hz, 1H), 4.97 (s, 0H), 3.97 (s, 4H), 2.36 (s, 3H), 2.06 (s, 2H), 1.84 (d, J = 11.9 Hz, 2H). |
| 251 | 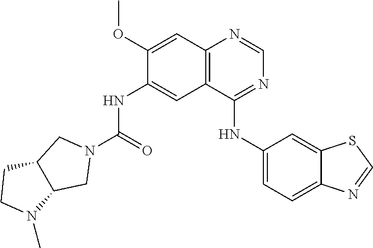 [SMILES: COc1cc2ncnc(Nc3ccc4ncsc4c3)c2cc1NC(=O)N1C[C@H]2CCN(C)[C@H]2C1] | (3aR,6aR)-N-(4-(benzo[d]thiazol-6-ylamino)-7-methoxyquinazolin- 6-yl)-1-methylhexahydropyrrolo[3,4-b]pyrrole-5(1H)-carboxamide LC-MS: (ES, m/z): RT = 1.970 min, LC-MS: m/z = 476.2 [M + 1]. ¹H NMR (400MHz, Chloroform-d) δ 8.85 (s, 1H), 8.81 (s, 1H), 8.66 (d, 1H, J = 1.6 Hz,), 8.59 (s, 1H), 8.02 (d, 1H, J = 8.8 Hz,), 7.91 (s, 1H), 7.61 (d, 1H, J = 8.8,1.6Hz), 7.17 (s, 1H), 7.13 (s, 1H), 3.98 (s, 3H), 3.72-3.64 (m, 1H), 3.63 (d, 1H, J = 10.4 Hz,), 3.46 (d, 1H, J = 10.8,4.4 Hz), 3.33 (d, 1H, J = 9.6,5.6 Hz), 3.14 (t, 1H, J = 8.4 Hz,), 2.84 (s, 2H), 2.34-2.30 (m, 4H), 2.27-2.04 (m, 1H), 1.74-1.72 (m, 1H). |
| 252 | 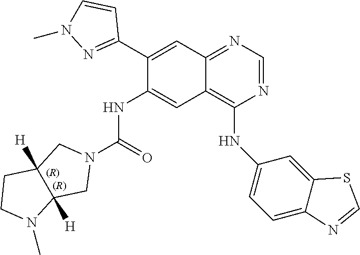 | (3aR,6aR)-N-(4-(benzo[d]thiazol-6-ylamino)-7-(1-methyl-1H- pyrazol-3-yl)quinazolin-6-yl)-1-methylhexahydropyrrolo[3,4- b]pyrrole-5(1H)-carboxamide LC-MS: (ES, m/z): RT = 2.233 min, LC-MS: m/z = 526.2 [M + 1]. ¹H NMR (400 MHz, DMSO-d₆) δ 10.78 (s, 1H), 10.02 (s, 1H), 9.30 (s, 1H), 9.17 (s, 1H), 8.69 (s, 1H), 8.53 (s, 1H), 8.21 (s, 1H), 8.08 (d, 1H, J = 8.8 Hz), 7.98 (d, 1H, J = 2.0 Hz), 7.92 (d, 1H, J = 7.2 Hz), 7.16 (d, 1H, J = 2.0 Hz), 4.01 (s, 3H), 3.79-3.77 (m, 1H), 3.68 (d, 1H, J = 11.2 Hz), 3.08-3.04 (m, 2H), 2.87 (s, 2H), 2.28-2.27 (m, 5H), 2.04-2.02 (m, 1H), 1.66-1.65 (m, 1H). |
| 253 | 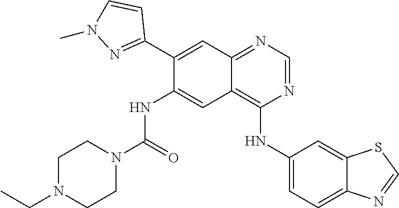 [SMILES: CCN1CCN(C(=O)Nc2cc3c(Nc4ccc5ncsc5c4)ncnc3cc2-c2ccn(C)n2)CC1] | N-(4-(benzo[d]thiazol-6-ylamino)-7-(1-methyl-1H-pyrazol-3- yl)quinazolin-6-yl)-4-ethylpiperazine-1-carboxamide LC-MS: (ES, m/z): RT = 2.258 min, LC-MS: m/z = 514.2 [M + 1]. ¹H NMR (400 MHz, DMSO-d6) δ 11.03 (s, 1H), 10.03 (s, 1H), 9.30 (s, 1H), 9.11 (s, 1H), 8.71 (s, 1H), 8.53 (s, 1H), 8.22 (s, 1H), 8.08 (d, 1H, J = 8.8 Hz), 7.98 (d, 1H, J = 2.0 Hz), 7.93 (d, 1H, J = 7.2 Hz), 7.17 (d, 1H, J = 2.0 Hz), 4.01 (s, 3H), 3.62 (s, 4H), 2.26-3.25 (m, 2H), 2.50-2.40 (m, 4H), 1.07-1.04 (m, 3H). |
| 254 | 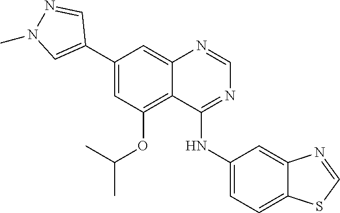 [SMILES: CC(C)Oc1cc(-c2cnn(C)c2)cc2ncnc(Nc3ccc4scnc4c3)c12] | N-(5-isopropoxy-7-(1-methyl-1H-pyrazol-4-yl)quinazolin-4- yl)benzo[d]thiazol-5-amine LC-MS: m/z = 417.1 [M + 1]. ¹H NMR (400 MHz, DMSO-d) δ 10.46 (s, 1H), 9.42 (s, 1H), 8.91 (d, 1H, J = 0.8 Hz), 8.57 (s, 1H), 8.45 (s, 1H), 8.17-8.14 (m, 2H), 7.70 (dd, 1H, J = 8.8, 2.0 Hz), 7.55 (s, 1H), 7.37 (s, 1H), 5.23-5.17 (m, 1H), 3.91 (s, 3H), 1.58 (d, 6H, J = 6.0 Hz). |
| 255 | 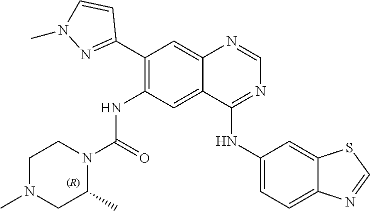 | (R)-N-(4-(benzo[d]thiazol-6-ylamino)-7-(1-methyl-1H-pyrazol-3- yl)quinazolin-6-yl)-2,4-dimethylpiperazine-1-carboxamide LC-MS: (ES, m/z):RT = 1.66 min, LC-MS: m/z = 514.2 [M + 1]. ¹H NMR (400 MHz, Chloroform-d) δ 11.06 (s, 1H), 9.26 (s, 1H), 8.95 (s, 1H), 8.77 (d, 1H, J = 1.8 Hz), 8.72 (s, 1H), 8.21 (s, 1H), 8.20-8.11 (m, 2H), 7.73 (dd, 1H, J = 8.8,2.0 Hz), 7.54 (d, 1H, J = 2.3 Hz), 6.89 (d, 1H, J = 2.3 Hz), 4.57 (s, 1H), 4.19 (d, 1H, J = 12.4 Hz), 4.03 (s, 3H), 3.43-3.37 (m, 1H), 2.92 (d, 1H, J = 11.4 Hz), 2.78 (d, 1H), 2.34-2.29 (m, 4H, J = 11.1 Hz), 2.12 (m, 1H, J = 11.7,3.0Hz), 1.47 (d, 3H, J = 6.8 Hz). |
| 256 | 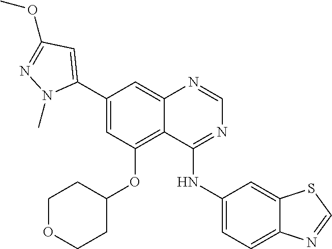 [SMILES: COc1cc(-c2cc(OC3CCOCC3)c3c(Nc4ccc5ncsc5c4)ncnc3c2)n(C)n1] | N-(7-(3-methoxy-1-methyl-1H-pyrazol-5-yl)-5-((tetrahydro-2H- pyran-4-yl)oxy)quinazolin-4-yl)benzo[d]thiazol-6-amine LC-MS: (ES, m/z): RT = 2.042 min, LC-MS: m/z = 489.1 [M + 1]. ¹H NMR (400 MHz, DMSO-d₆) δ 10.35 (s, 1H), 9.33 (s, 1H), 8.92 (d, 1H, J = 1.6 Hz), 8.64 (s, 1H), 8.13 (d, 1H, J = 8.8 Hz), 7.80 (dd, 1H, J = 8.8, 2.0 Hz), 7.47 (s, 1H), 7.37 (s, 1H), 6.11 (s, 1H), 5.18-5.14 (m, 1H), 4.02-3.89 (m, 2H), 3.82 (d, 6H, J = 7.6 Hz), 3.59 (t, 2H, J = 10.4 Hz), 2.27 (d, 2H, J = 12.0 Hz), 2.00-1.84 (m, 2H). |
| 257 | 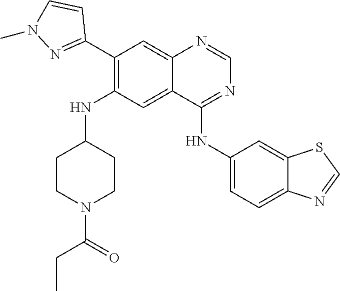 [SMILES: CCC(=O)N1CCC(Nc2cc3c(Nc4ccc5ncsc5c4)ncnc3cc2-c2ccn(C)n2)CC1] | 1-(4-((4-(benzo[d]thiazol-6-ylamino)-7-(1-methyl-1H-pyrazol-3- yl)quinazolin-6-yl)amino)piperidin-1-yl)propan-1-one LC-MS: (ES, m/z): RT = 2.705 min, LC-MS: m/z = 513.2 [M + 1]. ¹H NMR (400 MHz, DMSO-d6) δ 9.62 (s, 1 H), 9.32 (s, 1 H), 8.72 (s, 1 H), 8.38 (s, 1 H), 8.19 (d, 1 H, J = 8.00 Hz), 8.12 (d, 1 H, J = 8.80 Hz), 8.07 (s, 1 H), 7.92 (s, 1 H), 7.89 (s, 1 H), 7.48 (s, 1 H), 7.04 (s, 1 H), 4.05 (s, 2 H), 3.97 (s, 3 H), 3.76 (s, 1H), 2.67 (s, 2 H), 2.33 (s, 2 H), 2.17 (s, 2 H), 1.52 (s, 2 H), 1.02 (t, 3 H, J = 7.60 Hz). |
| 258 | 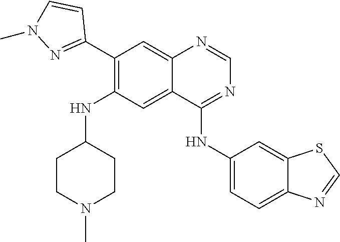 [SMILES: CN1CCC(Nc2cc3c(Nc4ccc5ncsc5c4)ncnc3cc2-c2ccn(C)n2)CC1] | N4-(benzo[d]thiazol-6-yl)-7-(1-methyl-1H-pyrazol-3-yl)-N6-(1- methylpiperidin-4-yl)quinazoline-4,6-diamine LC-MS: (ES, m/z): RT = 2.20 min, LC-MS: m/z = 471.1 [M + 1]. ¹H NMR (400 MHz, DMSO-d6) δ 9.60 (s, 1H), 9.31 (s, 1H), 8.72 (s, 1H), 8.39 (m, 1H), 8.12-8.08 (m, 3H), 7.90 (s, 2H), 7.45 (s, 1H), 7.06 (s, 1H), 3.99 (s, 3H), 3.39 (s, 4H), 2.66 (s, 3H), 2.53 (s, 3H). |
| 259 | 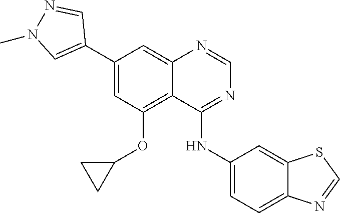 [SMILES: Cn1cc(-c2cc(OC3CC3)c3c(Nc4ccc5ncsc5c4)ncnc3c2)cn1] | N-(5-cyclopropoxy-7-(1-methyl-1H-pyrazol-4-yl)quinazolin-4- yl)benzo[d]thiazol-6-amine LC-MS: (ES, m/z): RT = 2.670 min, LC-MS: m/z = 415 [M + 1]; ¹H NMR (500 MHz, DMSO-d6) δ 9.95 (s, 1H), 9.33 (s, 1H), 8.89 (d, J = 2.1 Hz, 1H), 8.58 (s, 1H), 8.47 (s, 1H), 8.15 (s, 1H), 8.12 (d, J = 8.7 Hz, 1H), 7.79 (dd, J = 8.8, 2.2 Hz, 1H), 7.67 (d, J = 1.7 Hz, 1H), 7.63 (d, J = 1.5 Hz, 1H), 4.35 (dp, J = 5.9, 3.4 Hz, 1H), 3.94 (s, 3H), 1.09 (p, J = 2.9 Hz, 4H). |
| 260 | 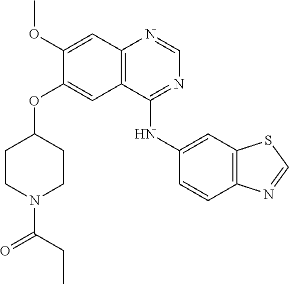 [SMILES: CCC(=O)N1CCC(Oc2cc3c(Nc4ccc5ncsc5c4)ncnc3cc2OC)CC1] | 1-(4-((4-(benzo[d]thiazol-6-ylamino)-7-methoxyquinazolin-6- yl)oxy)piperidin-1-yl)propan-1-one LC-MS: (ES, m/z): RT = 2.009 min, LC-MS: m/z = 463.1 [M + 1]. ¹H NMR (400 MHz, CD₃OD) δ 9.18 (s, 1H), 8.62 (d, 1H, J = 2.0 Hz), 8.45 (s, 1H), 8.06 (d, 1H, J = 8.8 Hz), 7.88 (s, 1H), 7.80 (dd, 1H, J = 8.8, 2.0 Hz), 7.19 (s, 1H), 4.00 (s, 3H), 3.91-3.83 (m, 2H), 3.63-3.51 (m, 2H), 2.48-2.43 (m, 2H), 2.11-1.83 (m, 5H), 1.14 (t, 3H, J = 7.6 Hz). |
| 261 | 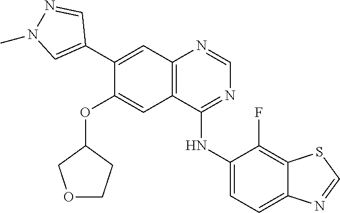 | 7-fluoro-N-(7-(1-methyl-1H-pyrazol-4-yl)-6-((tetrahydrofuran-3- yl)oxy)quinazolin-4-yl)benzo[d]thiazol-6-amine LC-MS: (ES, m/z): RT = 1.286 min, LCMS: m/z = 460 [M + 1], ¹H NMR (300 MHz, DMSO-d6) δ 9.89 (s, 1H), 9.49 (d, J = 1.5 Hz, 1H), 8.39 (s, 1H), 8.32 (s, 1H), 8.13 (s, 1H), 8.09-8.00 (m, 2H), 7.90 (s, 1H), 7.80-7.69 (m, 1H), 5.36 (s, 1H), 4.15-3.78 (m, 7H), 2.49- 2.38 (m, 1H), 2.26-2.15 (m, 1H). |
| 262 | 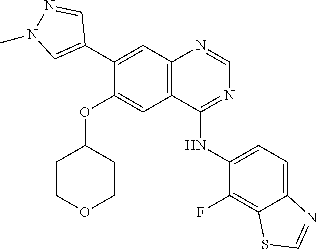 [SMILES: Cn1cc(-c2cc3ncnc(Nc4ccc5ncsc5c4F)c3cc2OC2CCOCC2)cn1] | 7-fluoro-N-(7-(1-methyl-1H-pyrazol-4-yl)-6-((tetrahydro-2H-pyran- 4-yl)oxy)quinazolin-4-yl)benzo[d]thiazol-6-amine LC-MS: (ES, m/z): RT = 1.341 min, LCMS: m/z = 477 [M + 1], ¹H NMR (300 MHz, DMSO-d6) δ 9.83 (s, 1H), 9.49 (d, J = 1.5 Hz, 1H), 8.36 (d, J = 7.1 Hz, 2H), 8.14 (s, 1H), 8.05 (d, J = 8.0 Hz, 2H), 7.96 (s, 1H), 7.74 (t, J = 8.1 Hz, 1H), 4.97-4.88 (m, 1H), 3.94 (s, 5H), 3.58 (s, 2H), 2.19 (d, J = 12.9 Hz, 2H), 1.84 (d, J = 9.0 Hz, 2H) .. |
| 263 | 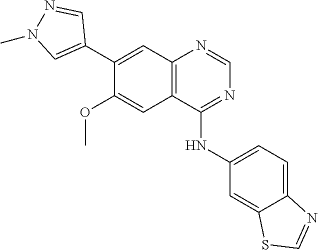 [SMILES: COc1cc2c(Nc3ccc4ncsc4c3)ncnc2cc1-c1cnn(C)c1] | N-(6-methoxy-7-(1-methyl-1H-pyrazol-4-yl)quinazolin-4- yl)benzo[d]thiazol-6-amine LC-MS: (ES, m/z): RT = 1.353 min, LCMS: m/z = 389 [M + 1], ¹H NMR (300 MHz, DMSO-d6) δ 9.88 (s, 1H), 9.33 (s, 1H), 8.75 (d, J = 2.1 Hz, 1H), 8.54 (s, 1H), 8.39 (s, 1H), 8.18-8.08 (m, 2H), 8.03 (d, J = 13.4 Hz, 2H), 7.92 (dd, J = 8.8, 2.2 Hz, 1H), 4.11 (s, 3H), 3.93 (s, 3H). |
| 264 | 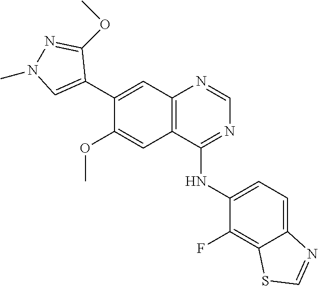 [SMILES: COc1cc2c(Nc3ccc4ncsc4c3F)ncnc2cc1-c1cn(C)nc1OC] | 7-fluoro-N-(6-methoxy-7-(3-methoxy-1-methyl-1H-pyrazol-4- yl)quinazolin-4-yl)benzo[d]thiazol-6-amine LC-MS: (ES, m/z): RT = 1.380 min, LCMS: m/z = 437 [M + 1], ¹H NMR (300 MHz, DMSO-d6) δ 9.88 (s, 1H), 9.48 (d, J = 1.5 Hz, 1H), 8.40-8.20 (m, 3H), 8.04 (d, J = 8.6 Hz, 1H), 7.92 (s, 1H), 7.73 (t, J = 8.1 Hz, 1H), 4.03 (d, J = 21.1 Hz, 6H), 3.81 (s, 3H). |
| 265 | 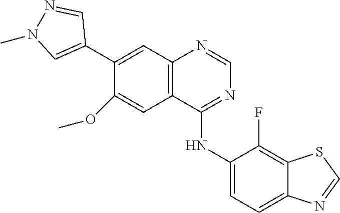 [SMILES: COc1cc2c(Nc3ccc4ncsc4c3F)ncnc2cc1-c1cnn(C)c1] | 7-fluoro-N-(6-methoxy-7-(1-methyl-1H-pyrazol-4-yl)quinazolin-4- yl)benzo[d]thiazol-6-amine LC-MS: (ES, m/z): RT = 1.207 min, LCMS: m/z = 407 [M + 1], ¹H NMR (300 MHz, DMSO-d6) δ 9.91 (s, 1H), 9.48 (d, J = 1.5 Hz, 1H), 8.39 (d, J = 1.8 Hz, 2H), 8.15 (d, J = 0.7 Hz, 1H), 8.09-8.00 (m, 2H), 7.96 (s, 1H), 7.74 (dd, J = 8.6, 7.5 Hz, 1H), 4.09 (s, 3H), 3.93 (s, 3H). |
| 266 | 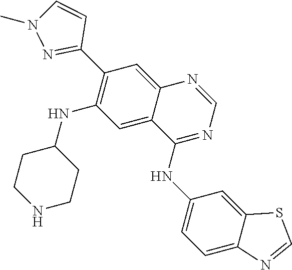 [SMILES: Cn1ccc(-c2cc3ncnc(Nc4ccc5ncsc5c4)c3cc2NC2CCNCC2)n1] | N4-(benzo[d]thiazol-6-yl)-7-(1-methyl-1H-pyrazol-3-yl)-N6- (piperidin-4-yl)quinazoline-4,6-diamine LC-MS: (ES, m/z): RT = 0.819 min, LCMS: m/z = 457 [M + 1], ¹H NMR (400 MHz, DMSO-d₆) δ 9.62 (s, 1H), 9.29 (s, 1H), 8.69 (d, J = 2.1 Hz, 1H), 8.35 (s, 1H), 8.09 (dd, J = 24.2, 15.4 Hz, 3H), 7.96- 7.78 (m, 2H), 7.43 (s, 1H), 7.01 (d, J = 2.5 Hz, 1H), 3.96 (s, 4H), 3.10-2.86 (m, 2H), 2.73 (t, J = 10.9 Hz, 2H), 2.18-1.91 (m, 2H), 1.43 (q, J = 10.5, 10.0 Hz, 2H). |
| 270 | 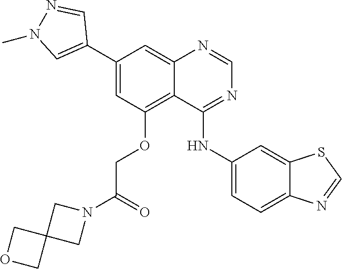 [SMILES: Cn1cc(-c2cc(OCC(=O)N3CC4(COC4)C3)c3c(Nc4ccc5ncsc5c4)ncnc3c2)cn1] | 2-((4-(benzo[d]thiazol-6-ylamino)-7-(1-methyl-1H-pyrazol-4- yl)quinazolin-5-yl)oxy)-1-(2-oxa-6-azaspiro[3.3]heptan-6-yl)ethan- 1-one LC-MS: (ES, m/z): RT = 1.163 min, LCMS: m/z = 514.4 [M + 1], ¹H NMR (300 MHz, DMSO-d6) δ 9.29 (s, 1H), 8.57 (s, 1H), 8.42 (s, 1H), 8.15-8.10 (m, 3H), 7.58 (s, 1H), 7.34 (s, 1H), 4.98 (s, 2H), 4.71 (s, 4H), 4.44 (s, 2H), 4.25 (s, 2H), 3.92 (s, 3H). |
| 271 | 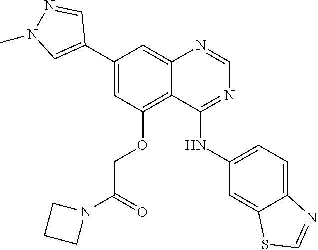 [SMILES: Cn1cc(-c2cc(OCC(=O)N3CCC3)c3c(Nc4ccc5ncsc5c4)ncnc3c2)cn1] | 1-(azetidin-1-yl)-2-((4-(benzo[d]thiazol-6-ylamino)-7-(1-methyl-1H- pyrazol-4-yl)quinazolin-5-yl)oxy)ethan-1-one LC-MS: (ES, m/z): RT = 1.241 min, LCMS: m/z = 472.3 [M + 1], ¹H NMR (300 MHz, DMSO-d6) δ 11.05 (s, 1H), 9.29 (s, 1H), 9.12 (d, J = 1.8 Hz, 1H), 8.56 (s, 1H), 8.40 (s, 1H), 8.14-8.09 (m, 3H), 7.57 (s, 1H), 7.31 (s, 1H), 4.97 (s, 2H), 4.27 (s, 2H), 4.06 (t, J = 7.7 Hz, 2H), 3.91 (s, 3H), 2.34 (t, J = 7.7 Hz, 2H). |
| 272 | 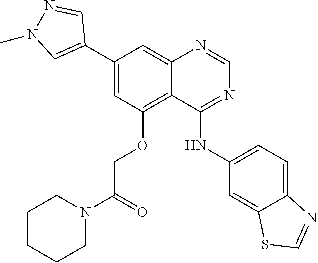 [SMILES: Cn1cc(-c2cc(OCC(=O)N3CCCCC3)c3c(Nc4ccc5ncsc5c4)ncnc3c2)cn1] | 2-((4-(benzo[d]thiazol-6-ylamino)-7-(1-methyl-1H-pyrazol-4- yl)quinazolin-5-yl)oxy)-1-(piperidin-1-yl)ethan-1-one LC-MS: (ES, m/z): RT = 1.512 min, LCMS: m/z = 500.4 [M + 1], ¹H NMR (300 MHz, DMSO-d6) δ 11.31 (s, 1H), 9.29 (s, 1H), 8.56 (s, 1H), 8.41 (s, 1H), 8.17-8.09 (m, 3H), 7.56 (s, 1H), 7.56 (s, 1H), 5.18 (s, 2H), 3.92 (s, 3H), 3.60 (s, 2H), 3.55-3.47 (m, 2H), 1.64 (s, 5H), 1.55 (s, 1H). |
| 273 | 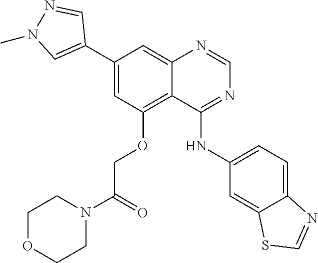 [SMILES: Cn1cc(-c2cc(OCC(=O)N3CCOCC3)c3c(Nc4ccc5ncsc5c4)ncnc3c2)cn1] | 2-((4-(benzo[d]thiazol-6-ylamino)-7-(1-methyl-1H-pyrazol-4- yl)quinazolin-5-yl)oxy)-1-morpholinoethan-1-one LC-MS: (ES, m/z): RT = 1.267 min, LCMS: m/z = 502.4 [M + 1], ¹H NMR (300 MHz, DMSO-d6) δ 11.24 (s, 1H), 9.29 (s, 1H), 9.13 (d, J = 2.1 Hz, 1H), 8.57 (s, 1H), 8.40 (s, 1H), 8.24-8.13 (m, 3H), 7.58 (d, J = 1.4 Hz, 1H), 7.40 (d, J = 1.6 Hz, 1H), 5.21 (s, 2H), 3.91 (s, 3H), 3.34-3.32 (m, 8H). |
| 274 | 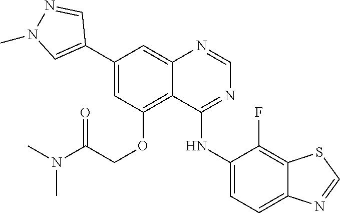 [SMILES: CN(C)C(=O)COc1cc(-c2cnn(C)c2)cc2ncnc(Nc3ccc4ncsc4c3F)c12] | 2-((4-((7-fluorobenzo[d]thiazol-6-yl)amino)-7-(1-methyl-1H- pyrazol-4-yl)quinazolin-5-yl)oxy)-N,N-dimethylacetamide LC-MS: (ES, m/z): RT = 1.190 min, LCMS: m/z = 478.4 [M + 1], ¹H NMR (300 MHz, DMSO-d6) δ 10.99 (s, 1H), 9.47 (d, J = 1.5 Hz, 1H), 8.42 (d, J = 16.8 Hz, 2H), 8.16 (s, 1H), 8.02 (d, J = 8.7 Hz, 1H), 7.80-7.75 (m, 1H), 7.57 (s, 1H), 7.41 (s, 1H), 5.22 (s, 2H), 3.92 (s, 3H), 3.06 (s, 3H), 2.89 (s, 3H). |
| 275 | 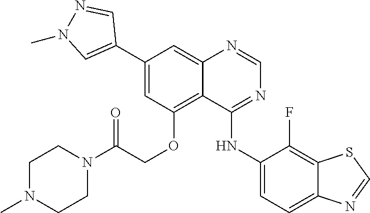 [SMILES: CN1CCN(C(=O)COc2cc(-c3cnn(C)c3)cc3ncnc(Nc4ccc5ncsc5c4F)c23)CC1] | 2-((4-((7-fluorobenzo[d]thiazol-6-yl)amino)-7-(1-methyl-1H- pyrazol-4-yl)quinazolin-5-yl)oxy)-1-(4-methylpiperazin-1-yl)ethan- 1-one LC-MS: (ES, m/z): RT = 1.150 min, LCMS: m/z = 533.4 [M + 1], ¹H NMR (300 MHz, DMSO-d6) δ 10.89 (s, 1H), 9.40 (d, J = 1.5 Hz, 1H), 8.33 (d, J = 15.0 Hz, 2H), 8.03 (d, J = 0.8 Hz, 1H), 7.80 (d, J = 8.6 Hz, 1H), 7.72-7.50 (m, 1H), 7.35 (s, 1H), 7.34 (s, 1H), 5.15 (s, 2H), 3.85 (s, 3H), 3.43 (d, J = 3.6 Hz, 1H), 3.29 (s, 2H), 2.33 (s, 2H), 2.22 (s, 3H), 2.13 (s, 3H). |
| 276 | 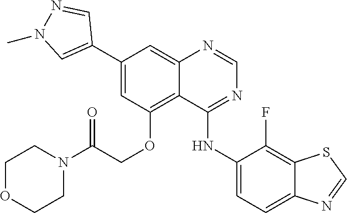 [SMILES: Cn1cc(-c2cc(OCC(=O)N3CCOCC3)c3c(Nc4ccc5ncsc5c4F)ncnc3c2)cn1] | 2-((4-((7-fluorobenzo[d]thiazol-6-yl)amino)-7-(1-methyl-1H- pyrazol-4-yl)quinazolin-5-yl)oxy)-1-morpholinoethan-1-one LC-MS: (ES, m/z): RT = 1.186 min, LCMS: m/z = 520.4 [M + 1], ¹H NMR (300 MHz, DMSO-d6) δ 10.93 (s, 1H), 9.47 (d, J = 1.5 Hz, 1H), 8.40 (d, J = 11.7 Hz, 2H), 8.15 (s, 1H), 8.02 (d, J = 8.7 Hz, 1H), 7.80-7.58 (m, 1H), 7.41 (s, 1H), 7.41 (s, 1H), 5.24 (s, 2H), 3.92 (s, 3H), 3.69 (s, 2H), 3.59-3.52 (m, 6H). |
| 277 | 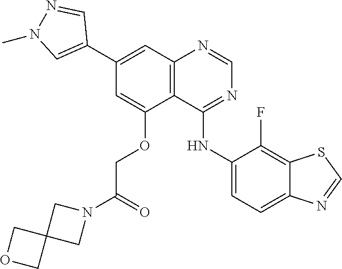 [SMILES: Cn1cc(-c2cc(OCC(=O)N3CC4(COC4)C3)c3c(Nc4ccc5ncsc5c4F)ncnc3c2)cn1] | 2-((4-((7-fluorobenzo[d]thiazol-6-yl)amino)-7-(1-methyl-1H- pyrazol-4-yl)quinazolin-5-yl)oxy)-1-(2-oxa-6-azaspiro[3.3]heptan-6- yl)ethan-1-one LC-MS: (ES, m/z): RT = 1.107 min, LCMS: m/z = 532.4 [M + 1], ¹H NMR (300 MHz, DMSO-d6) δ 10.71 (s, 1H), 9.46 (d, J = 1.5 Hz, 1H), 8.40 (d, J = 11.7 Hz, 2H), 8.14 (d, J = 0.6 Hz, 1H), 8.01 (d, J = 8.7 Hz, 1H), 7.82-7.58 (m, 1H), 7.34 (s, 1H), 7.34 (s, 1H), 5.00 (s, 2H), 4.68 (s, 4H), 4.41 (s, 2H), 4.14 (s, 2H), 3.92 (s, 3H). |
| 278 | 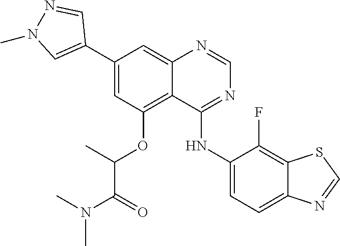 [SMILES: CC(Oc1cc(-c2cnn(C)c2)cc2ncnc(Nc3ccc4ncsc4c3F)c12)C(=O)N(C)C] | 2-((4-((7-fluorobenzo[d]thiazol-6-yl)amino)-7-(1-methyl-1H- pyrazol-4-yl)quinazolin-5-yl)oxy)-N,N-dimethylpropanamide LC-MS: (ES, m/z): RT = 1.373 min, LCMS: m/z = 492 [M + 1], ¹H NMR (300 MHz, DMSO-d6) δ ppm 9.44 (d, J = 1.6 Hz, 1H), 8.42 (d, J = 11.3 Hz, 2H), 8.19 (dd, J = 8.8, 7.6 Hz, 1H), 8.13 (s, 1H), 8.03 (d, J = 8.8 Hz, 1H), 7.58 (d, J = 1.4 Hz, 1H), 7.44 (d, J = 1.5 Hz, 1H), 5.96 (q, J = 6.3 Hz, 1H), 3.93 (s, 3H), 3.17 (s, 3H), 2.87 (s, 3H), 1.64 (d, J = 6.3 Hz, 3H). |
| 279 280 | 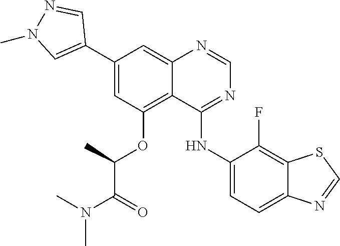 [SMILES: C[C@@H](Oc1cc(-c2cnn(C)c2)cc2ncnc(Nc3ccc4ncsc4c3F)c12)C(=O)N(C)C] or 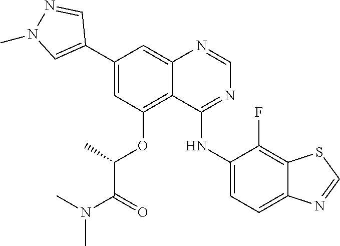 [SMILES: C[C@H](Oc1cc(-c2cnn(C)c2)cc2ncnc(Nc3ccc4ncsc4c3F)c12)C(=O)N(C)C] | (R)-2-((4-((7-fluorobenzo[d]thiazol-6-yl)amino)-7-(1-methyl-1H- pyrazol-4-yl)quinazolin-5-yl)oxy)-N,N-dimethylpropanamide OR (S)-2-((4-((7-fluorobenzo[d]thiazol-6-yl)amino)-7-(1-methyl-1H- pyrazol-4-yl)quinazolin-5-yl)oxy)-N,N-dimethylpropanamide First Eluting Isomer: LC-MS: (ES, m/z): RT = 1.339 min, LCMS: m/z = 492 [M + 1], Chiral-HPLC (ES): RT = 2.164, ¹H NMR (300 MHz, DMSO-d6) δ ppm 10.97 (s, 1H), 9.45 (s, 1H), 8.42 (d, J = 4.7 Hz, 2H), 8.14 (s, 2H), 8.02 (d, J = 8.7 Hz, 1H), 7.58 (s, 1H), 7.45 (s, 1H), 5.96 (d, J = 6.4 Hz, 1H), 3.93 (d, J = 2.0 Hz, 3H), 3.17 (d, J = 2.0 Hz, 3H), 2.87 (d, J = 2.0 Hz, 3H), 1.63 (d, J = 6.1 Hz, 3H). Second Eluting Isomer: LC-MS: (ES, m/z): RT = 1.356 min, LCMS: m/z = 492 [M + 1], Chiral-HPLC (ES): RT = 2.749, ¹H NMR (300 MHz, DMSO-d6) δ ppm 10.96 (s, 1H), 9.45 (s, 1H), 8.42 (d, J = 4.7 Hz, 2H), 8.14 (s, 2H), 8.02 (d, J = 8.7 Hz, 1H), 7.56 (s, 1H), 7.44 (s, 1H), 5.96 (d, J = 6.4 Hz, 1H), 3.93 (d, J = 2.0 Hz, 3H), 3.17 (d, J = 2.0 Hz, 3H), 2.86 (d, J = 2.0 Hz, 3H), 1.62 (d, J = 6.1 Hz, 3H) |
| 281 | 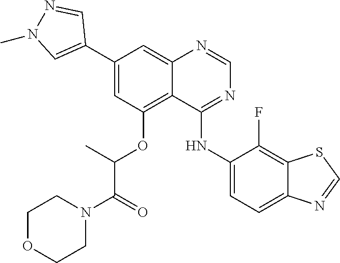 [SMILES: CC(Oc1cc(-c2cnn(C)c2)cc2ncnc(Nc3ccc4ncsc4c3F)c12)C(=O)N1CCOCC1] | 2-((4-((7-fluorobenzo[d]thiazol-6-yl)amino)-7-(1-methyl-1H- pyrazol-4-yl)quinazolin-5-yl)oxy)-1-morpholinopropan-1-one LCMS: m/z = 534 [M + 1]. ¹H NMR (300 MHz, DMSO-d6) δ ppm 9.45 (d, J = 1.5 Hz, 1H), 8.42 (d, J = 13.2 Hz, 2H), 8.23-8.10 (m, 2H), 8.03 (d, J = 8.7 Hz, 1H), 7.58 (s, 1H), 7.43 (s, 1H), 5.98 (d, J = 6.6 Hz, 1H), 3.93 (s, 3H), 3.68-3.61 (m, 8H), , 1.64 (d, J = 6.3 Hz, 3H). |
| 282 283 | 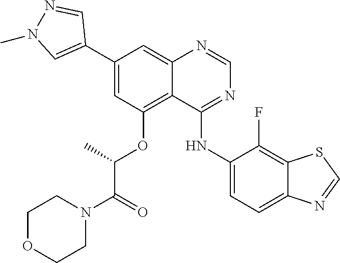 [SMILES: C[C@H](Oc1cc(-c2cnn(C)c2)cc2ncnc(Nc3ccc4ncsc4c3F)c12)C(=O)N1CCOCC1] or 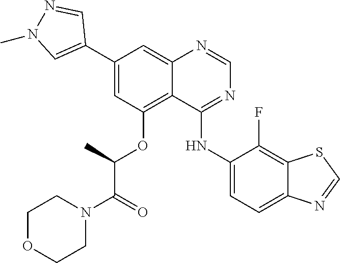 [SMILES: C[C@@H](Oc1cc(-c2cnn(C)c2)cc2ncnc(Nc3ccc4ncsc4c3F)c12)C(=O)N1CCOCC1] | (R)-2-((4-((7-fluorobenzo[d]thiazol-6-yl)amino)-7-(1-methyl-1H- pyrazol-4-yl)quinazolin-5-yl)oxy)-1-morpholinopropan-1-one OR (S)-2-((4-((7-fluorobenzo[d]thiazol-6-yl)amino)-7-(1-methyl-1H- pyrazol-4-yl)quinazolin-5-yl)oxy)-1-morpholinopropan-1-one First Eluting Isomer: LC-MS: (ES, m/z): RT = 1.339 min, LCMS: m/z = 534 [M + 1], Chiral-HPLC (ES): RT = 2.227, ¹H NMR (300 MHz, DMSO-d6) δ ppm 10.93 (s, 1H), 9.46 (d, J = 1.6 Hz, 1H), 8.54 (s, 0.4H), 8.43 (d, J = 8.3 Hz, 2H), 8.20-8.08 (m, 2H), 8.03 (d, J = 8.7 Hz, 1H), 7.59 (s, 1H), 7.44 (s, 1H), 5.98 (d, J = 6.4 Hz, 1H), 3.93 (s, 3H), 3.71-3.61 (m, 8H), 1.64 (d, J = 6.3 Hz, 3H). Second Eluting Isomer: LC-MS: (ES, m/z): RT = 1.356 min, LCMS: m/z = 534 [M + 1], Chiral-HPLC (ES): RT = 3.093, ¹H NMR (300 MHz, DMSO-d6) δ ppm 10.90 (s, 1H), 9.46 (d, J = 1.6 Hz, 1H), 8.43 (d, J = 8.3 Hz, 2H), 8.20-8.08 (m, 2H), 8.03 (d, J = 8.7 Hz, 1H), 7.55 (s, 1H), 7.44 (s, 1H), 5.98 (d, J = 6.4 Hz, 1H), 3.93 (s, 3H), 3.71- 3.61 (m, 8H), 1.63 (d, J = 6.3 Hz, 3H). |
| 284 | 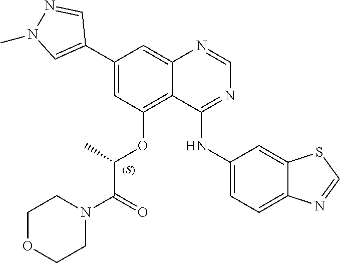 | (S)-2-((4-(benzo[d]thiazol-6-ylamino)-7-(1-methyl-1H-pyrazol-4- yl)quinazolin-5-yl)oxy)-1-morpholinopropan-1-one LC-MS: (ES, m/z): RT = 1.292 min, LCMS: m/z = 516 [M + 1], 1H NMR (300 MHz, DMSO-d6) δ 11.30 (s, 1H), 9.32 (s, 1H), 9.10- 9.02 (m, 1H), 8.59 (d, J = 1.3 Hz, 1H), 8.43 (s, 1H), 8.20-8.05 (m, 3H), 7.53 (dd, J = 27.4, 1.5 Hz, 2H), 5.99 (q, J = 6.3 Hz, 1H), 3.94 (s, 3H), 3.82-3.47 (m, 8H), 1.64 (d, J = 6.3 Hz, 3H). |
| 285 | 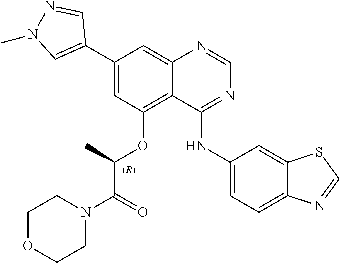 | (R)-2-((4-(benzo[d]thiazol-6-ylamino)-7-(1-methyl-1H-pyrazol-4- yl)quinazolin-5-yl)oxy)-1-morpholinopropan-1-one LC-MS: (ES, m/z): RT = 1.299 min, LCMS: m/z = 516 [M + 1], ¹H NMR (300 MHz, DMSO-d6) δ 11.24 (s, 1H), 9.31 (s, 1H), 9.11- 9.04 (m, 1H), 8.57 (s, 1H), 8.43 (s, 1H), 8.24-8.04 (m, 3H), 7.58 (d, J = 1.4 Hz, 1H), 7.47 (d, J = 1.5 Hz, 1H), 5.98 (d, J = 6.4 Hz, 1H), 3.93 (s, 3H), 3.84-3.48 (m, 8H), 1.64 (d, J = 6.3 Hz, 3H). |
| 286 | 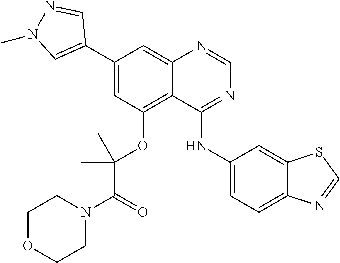 [SMILES: Cn1cc(-c2cc(OC(C)(C)C(=O)N3CCOCC3)c3c(Nc4ccc5ncsc5c4)ncnc3c2)cn1] | 2-((4-(benzo[d]thiazol-6-ylamino)-7-(1-methyl-1H-pyrazol-4- yl)quinazolin-5-yl)oxy)-2-methyl-1-morpholinopropan-1-one LC-MS: (ES, m/z): RT = 1.153 min, LCMS: m/z = 530 [M + 1],¹H NMR (400 MHz, DMSO-d6) 1H δ 10.10 (s, 1H), 9.33 (s, 1H), 8.93 (d, J = 2.1 Hz, 1H), 8.61 (s, 1H), 8.21 (s, 1H), 8.13 (d, J = 8.8 Hz, 1H), 7.91-7.81 (m, 2H), 7.65 (s, 1H), 7.01 (s, 1H), 3.93 (s, 3H), 3.87-3.72 (m, 2H), 3.70-3.58 (m, 2H), 3.54-3.43 (m, 2H), 3.22- 3.10 (m, 2H), 1.89 (s, 6H). |
| 287 288 | 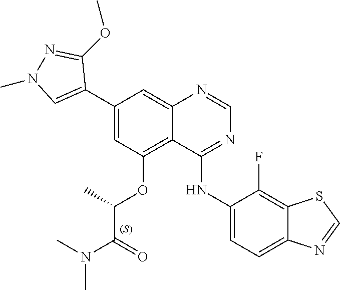 or 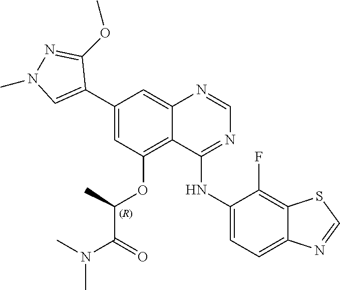 | (S)-2-((4-((7-fluorobenzo[d]thiazol-6-yl)amino)-7-(3-methoxy-1- methyl-1H-pyrazol-4-yl)quinazolin-5-yl)oxy)-N,N- dimethylpropanamide OR (R)-2-((4-((7-fluorobenzo[d]thiazol-6- yl)amino)-7-(3-methoxy-1-methyl-1H-pyrazol-4-yl)quinazolin-5- yl)oxy)-N,N-dimethylpropanamide First Eluting Isomer: LC-MS: (ES, m/z): RT = 1.439 min, LCMS: m/z = 522 [M + 1], Chiral-HPLC (ES): RT = 1.728, ¹H NMR (300 MHz, DMSO-d6) δ ppm 10.83 (s, 1H), 9.44 (d, J = 1.5 Hz, 1H), 8.45 (s, 1H), 8.36-8.25 (m, 2H), 8.03 (d, J = 8.8 Hz, 1H), 7.66 (d, J = 1.4 Hz, 1H), 7.38 (d, J = 1.5 Hz, 1H), 5.86 (q, J = 6.2 Hz, 1H), 3.99 (s, 3H), 3.79 (s, 3H), 3.18 (s, 3H), 2.88 (s, 3H), 1.65 (d, J = 6.4 Hz, 3H). Second Eluting Isomer: LC-MS: (ES, m/z): RT = 1.439 min, LCMS: m/z = 522 [M + 1], Chiral-HPLC (ES): RT = 2.256, ¹H NMR (300 MHz, DMSO-d6) δ ppm 10.83 (s, 1H), 9.44 (d, J = 1.6 Hz, 1H), 8.45 (s, 1H), 8.37-8.25 (m, 2H), 8.03 (d, J = 8.8 Hz, 1H), 7.66 (d, J = 1.4 Hz, 1H), 7.37 (d, J = 1.6 Hz, 1H), 5.86 (q, J = 6.3 Hz, 1H), 3.99 (s, 3H), 3.79 (s, 3H), 3.18 (s, 3H), 2.88 (s, 3H), 1.65 (d, J = 6.3 Hz, 3H). |
| 289 290 | 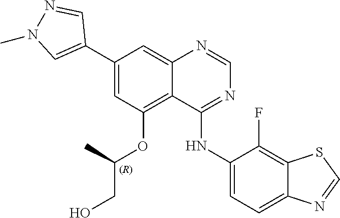 or 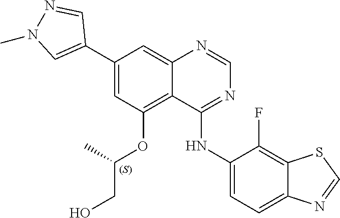 | (R)-2-((4-((7-fluorobenzo[d]thiazol-6-yl)amino)-7-(1-methyl-1H- pyrazol-4-yl)quinazolin-5-yl)oxy)propan-1-ol OR (S)-2-((4-((7- fluorobenzo[d]thiazol-6-yl)amino)-7-(1-methyl-1H-pyrazol-4- yl)quinazolin-5-yl)oxy)propan-1-ol First Eluting Isomer: LC-MS: (ES, m/z): RT = 1.358 min, m/z = 451 [M + 1], chiral-HPLC: R = 2.333 min, ¹H NMR (400 MHz, DMSO-d6) δ 10.41-10.36 (m, 1H), 9.43 (d, J = 1.5 Hz, 1H), 8.76-8.30 (m, 3H), 8.16 (d, J = 0.8 Hz, 1H), 8.02 (d, J = 8.9 Hz, 1H), 7.58 (d, J = 1.5 Hz, 1H), 7.46 (d, J = 1.7 Hz, 1H), 5.16 (dt, J = 43.5, 5.3 Hz, 2H), 3.92 (s, 3H), 3.86-3.54 (m, 2H), 1.46 (d, J = 6.2 Hz, 3H). Second Eluting Isomer: LC-MS: (ES, m/z): RT = 1.310 min, m/z = 451 [M + 1], chiral-HPLC: R = 2.766 min, ¹H NMR (400 MHz, DMSO-d6) δ 10.39 (s, 1H), 9.43 (d, J = 1.5 Hz, 1H), 8.54-8.40 (m, 3H), 8.16 (s, 1H), 8.02 (d, J = 8.9 Hz, 1H), 7.58 (d, J = 1.5 Hz, 1H), 7.46 (d, J = 1.6 Hz, 1H), 5.25-5.18 (m, 1H), 5.10 (q, J = 5.1 Hz, 1H), 3.91 (s, 3H), 3.85-3.78 (m, 1H), 3.73 (dt, J = 11.3, 4.9 Hz, 1H), 1.46 (d, J = 6.2 Hz, 3H). |
| 291 292 | 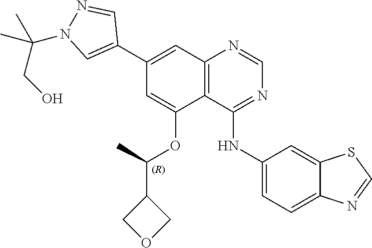 or 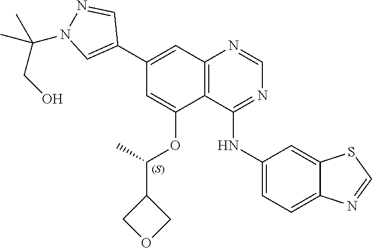 | (R)-2-(4-(4-(benzo[d]thiazol-6-ylamino)-5-(1-(oxetan-3- yl)ethoxy)quinazolin-7-yl)-1H-pyrazol-1-yl)-2-methylpropan-1-ol OR (S)-2-(4-(4-(benzo[d]thiazol-6-ylamino)-5-(1-(oxetan-3- yl)ethoxy)quinazolin-7-yl)-1H-pyrazol-1-yl)-2-methylpropan-1-ol First Eluting Isomer: LC-MS: (ES, m/z): RT = 0.977 min, LCMS: m/z = 517 [M + 1], HPLC: RT = 2.102 min; ¹H NMR (400 MHz, DMSO-d6) δ 10.16 (s, 1H), 9.32 (s, 1H), 8.77 (dd, J = 6.0, 2.2 Hz, 1H), 8.55 (d, J = 14.3 Hz, 2H), 8.19 (s, 1H), 8.09 (d, J = 8.8 Hz, 1H), 7.79 (dd, J = 8.9, 2.2 Hz, 1H), 7.65 (d, J = 1.4 Hz, 1H), 7.51 (d, J = 1.6 Hz, 1H), 5.44 (p, J = 6.1 Hz, 1H), 5.02 (t, J = 5.7 Hz, 1H), 4.85 (td, J = 7.9, 6.3 Hz, 2H), 4.66 (t, J = 6.1 Hz, 1H), 4.58 (t, J = 6.0 Hz, 1H), 3.65 (d, J = 5.7 Hz, 2H), 3.54 (q, J = 6.5, 5.8 Hz, 1H), 1.55 (s, 6H), 1.42 (d, J = 6.0 Hz, 3H). Second Eluting Isomer: LC-MS: (ES, m/z): RT = 0.971 min, LCMS: m/z = 517 [M + 1], HPLC: RT = 2.590 min; ¹H NMR (400 MHz, DMSO-d6) δ 10.16 (s, 1H), 9.33 (d, J = 0.8 Hz, 1H), 8.77 (dd, J = 6.0, 2.1 Hz, 1H), 8.59-8.51 (m, 2H), 8.19 (s, 1H), 8.09 (d, J = 8.8 Hz, 1H), 7.80 (dt, J = 8.9, 2.0 Hz, 1H), 7.65 (d, J = 1.4 Hz, 1H), 7.51 (s, 1H), 5.44 (p, J = 6.1 Hz, 1H), 5.02 (t, J = 5.6 Hz, 1H), 4.85 (td, J = 7.9, 6.4 Hz, 2H), 4.66 (t, J = 6.1 Hz, 1H), 4.58 (t, J = 6.0 Hz, 1H), 3.65 (d, J = 5.6 Hz, 2H), 3.57-3.49 (m, 1H), 1.55 (s, 6H), 1.42 (d, J = 6.0 Hz, 3H). |
| 293 294 | 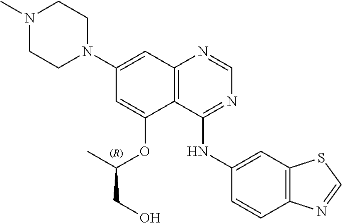 or 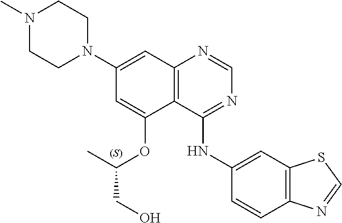 | (R)-2-((4-(benzo[d]thiazol-6-ylamino)-7-(4-methylpiperazin-1- yl)quinazolin-5-yl)oxy)propan-1-ol OR (S)-2-((4-(benzo[d]thiazol- 6-ylamino)-7-(4-methylpiperazin-1-yl)quinazolin-5-yl)oxy)propan-1- ol First Eluting Isomer: LC-MS: (ES, m/z): RT = 0.638 min, LCMS: m/z = 451.35 [M + 1], Chiral-HPLC (ES): RT = 5.113, ¹H NMR (300 MHz, DMSO-d6) δ 10.47 (s, 1H), 9.26 (s, 1H), 8.93 (d, J = 2.1 Hz, 1H), 8.42 (s, 1H), 8.03 (d, J = 8.8 Hz, 1H), 7.90 (dd, J = 8.9, 2.2 Hz, 1H), 6.87 (d, J = 2.2 Hz, 1H), 6.64 (d, J = 2.2 Hz, 1H), 5.44 (s, 1H), 4.99 (d, J = 6.3 Hz, 1H), 3.82-3.91 (m, 1H), 3.87 (s, 1H), 3.41- 3.53 (m, 6H), 3.22-3.34 (m, 2H), 2.27 (s, 3H), 1.41 (d, J = 6.1 Hz, 3H), 1.22 (s, 1H). Second Eluting Isomer: LC-MS: (ES, m/z): RT = 0.645 min, LCMS: m/z = 451.35 [M + 1], Chiral-HPLC (ES): RT = 6.284, ¹H NMR (300 MHz, DMSO-d6) δ 10.47 (s, 1H), 9.26 (s, 1H), 8.93 (d, J = 2.1 Hz, 1H), 8.41 (s, 1H), 8.03 (d, J = 8.9 Hz, 1H), 7.90 (dd, J = 8.9, 2.1 Hz, 1H), 6.87 (d, J = 2.3 Hz, 1H), 6.64 (d, J = 2.1 Hz, 1H), 5.44 (t, J = 5.1 Hz, 1H), 4.99 (s, 1H), 3.88 (d, J = 12.0 Hz, 1H), 3.73 (s, 1H), 3.42-3.63 (m, 4H), 3.22-3.36 (m, 4H), 2.24 (s, 3H), 1.41 (d, J = 6.1 Hz, 3H), 1.22 (s, 1H). |
| 295 296 | 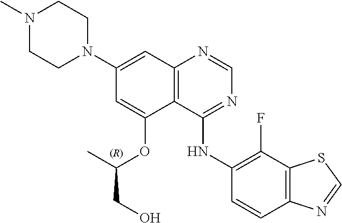 or 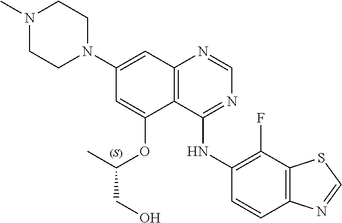 | (S)-2-((4-((7-fluorobenzo[d]thiazol-6-yl)amino)-7-(4- methylpiperazin-1-yl)quinazolin-5-yl)oxy)propan-1-ol OR (R)-2- ((4-((7-fluorobenzo[d]thiazol-6-yl)amino)-7-(4-methylpiperazin-1- yl)quinazolin-5-yl)oxy)propan-1-ol First Eluting Isomer: LC-MS: (ES, m/z): RT = 0.705 min, LCMS: m/z = 469.05 [M + 1], Chiral-HPLC (ES): RT = 1.893, ¹H NMR (400 MHz, Chloroform-d) δ 10.14 (s, 1H), 8.93-8.85 (m, 2H), 8.52 (s, 1H), 7.93 (d, J = 9.0 Hz, 1H), 6.74 (s, 1H), 6.66 (d, J = 2.2 Hz, 1H), 4.91 (s, 1H), 4.07 (dd, J = 12.2, 6.6 Hz, 2H), 3.43-3.36 (m, 4H), 2.61 (t, J = 5.1 Hz, 4H), 2.40 (s, 3H), 1.53 (d, J = 6.3 Hz, 3H). Second Eluting Isomer: LC-MS: (ES, m/z): RT = 0.940 min, LCMS: m/z = 469.05 [M + 1], Chiral-HPLC (ES): RT = 2.423, ¹H NMR (400 MHz, Chloroform-d) δ 10.12 (d, J = 2.5 Hz, 1H), 8.94-8.85 (m, 2H), 8.51 (s, 1H), 7.96-7.89 (m, 1H), 6.73 (d, J = 2.2 Hz, 2H), 4.90 (td, J = 6.5, 3.5 Hz, 1H), 4.04 (qd, J = 12.2, 5.0 Hz, 2H), 3.42-3.35 (m, 4H), 2.63-2.56 (m, 4H), 2.39 (s, 3H), 1.52 (d, J = 6.3 Hz, 3H). |
| 297 298 | 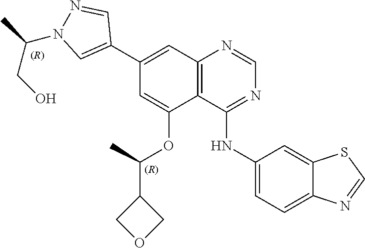 or 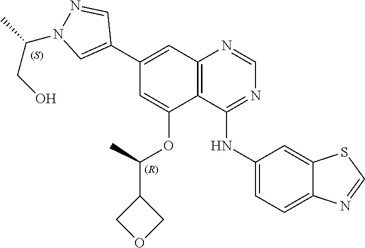 | (S)-2-(4-(4-(benzo[d]thiazol-6-ylamino)-5-((R)-1-(oxetan-3- yl)ethoxy)quinazolin-7-yl)-1H-pyrazol-1-yl)propan-1-ol, OR (R)-2- (4-(4-(benzo[d]thiazol-6-ylamino)-5-((R)-1-(oxetan-3- yl)ethoxy)quinazolin-7-yl)-1H-pyrazol-1-yl)propan-1-ol, First Eluting Isomer: LC-MS: (ES, m/z): RT = 0.788 min, LCMS: m/z = 503 [M + 1], Chiral-HPLC: 4.275, ¹H NMR (300 MHz, DMSO- d6) δ 10.15 (s, 1H), 9.31 (s, 1H), 8.74 (d, J = 2.1 Hz, 1H), 8.52 (d, J = 4.0 Hz, 2H), 8.17 (s, 1H), 8.07 (d, J = 8.8 Hz, 1H), 7.77 (dd, J = 8.8, 2.3 Hz, 1H), 7.59 (d, J = 1.3 Hz, 1H), 7.48 (s, 1H), 5.46-5.36 (m, 1H), 4.97 (d, J = 5.7 Hz, 1H), 4.83 (q, J = 6.4 Hz, 2H), 4.63 (t, J = 6.1 Hz, 1H), 4.55 (t, J = 6.0 Hz, 1H), 4.44-4.32 (m, 1H), 3.68 (dt, J = 9.9, 5.6 Hz, 2H), 3.51 (s, 2H), 1.42 (dd, J = 13.2, 6.4 Hz, 6H). Second Eluting Isomer: LC-MS: (ES, m/z): RT = 0.788 min, LCMS: m/z = 503 [M + 1], Chiral-HPLC: 4.275, ¹H NMR (300 MHz, DMSO- d6) δ 10.15 (s, 1H), 9.31 (s, 1H), 8.74 (d, J = 2.1 Hz, 1H), 8.52 (d, J = 4.0 Hz, 2H), 8.17 (s, 1H), 8.07 (d, J = 8.8 Hz, 1H), 7.77 (dd, J = 8.8, 2.3 Hz, 1H), 7.59 (d, J = 1.3 Hz, 1H), 7.48 (s, 1H), 5.46-5.36 (m, 1H), 4.97 (d, J = 5.7 Hz, 1H), 4.83 (q, J = 6.4 Hz, 2H), 4.63 (t, J = 6.1 Hz, 1H), 4.55 (t, J = 6.0 Hz, 1H), 4.44-4.32 (m, 1H), 3.68 (dt, J = 9.9, 5.6 Hz, 2H), 3.51 (s, 2H), 1.42 (dd, J = 13.2, 6.4 Hz, 6H). |
| 299 300 | 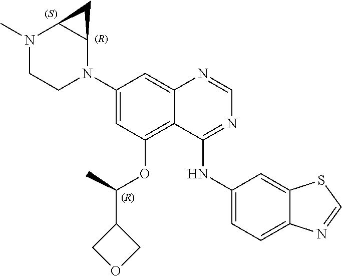 or 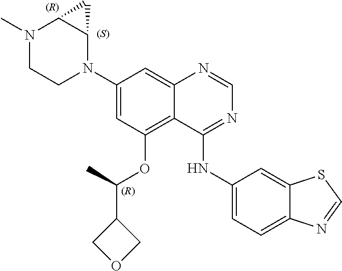 | N-(7-((1R,6S)-5-methyl-2,5-diazabicyclo[4.1.0]heptan-2-yl)-5-((R)- 1-(oxetan-3-yl)ethoxy)quinazolin-4-yl)benzo[d]thiazol-6-amine, OR N-(7-((1S,6R)-5-methyl-2,5-diazabicyclo[4.1.0]heptan-2-yl)-5-((R)- 1-(oxetan-3-yl)ethoxy)quinazolin-4-yl)benzo[d]thiazol-6-amine First Eluting Isomer: LC-MS: (ES, m/z): RT = 1.315 min, LCMS: m/z = 489 [M + 1]. Chiral-HPLC R = 1.760, ¹H NMR (400 MHz, DMSO-d6) δ 9.90 (s, 1H), 9.27 (s, 1H), 8.73 (d, J = 2.2 Hz, 1H), 8.34 (s, 1H), 8.03 (d, J = 8.8 Hz, 1H), 7.73 (dd, J = 8.8, 2.2 Hz, 1H), 6.81 (d, J = 2.2 Hz, 1H), 6.61 (d, J = 2.1 Hz, 1H), 5.26 (p, J = 6.0 Hz, 1H), 4.80 (td, J = 8.0, 6.3 Hz, 2H), 4.59 (t, J = 6.1 Hz, 1H), 4.52 (t, J = 6.0 Hz, 1H), 3.67-3.59 (m, 1H), 3.49 (d, J = 6.6 Hz, 1H), 3.29 (s, 1H), 2.84-2.75 (m, 1H), 2.70 (s, 1H), 2.61-2.52 (m, 1H), 2.40 (s, 3H), 2.33 (s, 1H), 1.38 (d, J = 6.0 Hz, 3H), 1.22 (s, 1H), 0.79 (q, J = 6.1 Hz, 1H), 0.40 (q, J = 4.9 Hz, 1H). Second Eluting Isomer: LC-MS: (ES, m/z): RT = 1.302 min, LCMS: m/z = 489 [M + 1]. Chiral-HPLC R = 2.624, 1H NMR (400 MHz, DMSO-d6) δ 9.92 (s, 1H), 9.27 (s, 1H), 8.74 (d, J = 2.2 Hz, 1H), 8.34 (s, 1H), 8.03 (d, J = 8.8 Hz, 1H), 7.73 (dd, J = 8.8, 2.2 Hz, 1H), 6.79 (d, J = 2.2 Hz, 1H), 6.63 (d, J = 2.1 Hz, 1H), 5.22 (p, J = 6.1 Hz, 1H), 4.79 (ddd, J = 10.4, 8.0, 6.3 Hz, 2H), 4.60 (t, J = 6.1 Hz, 1H), 4.53 (t, J = 6.0 Hz, 1H), 3.62-3.53 (m, 1H), 3.49 (d, J = 6.5 Hz, 1H), 3.30 (s, 2H), 2.80-2.67 (m, 1H), 2.60-2.50 (m, 1H), 2.40 (s, 3H), 2.39-2.30 (m, 1H), 1.98 (q, J = 7.3 Hz, 0H), 1.37 (d, J = 6.0 Hz, 3H), 1.21 (s, 2H), 0.87-0.76 (m, 1H), 0.42 (q, J = 4.9 Hz, 1H). |
| 301 | 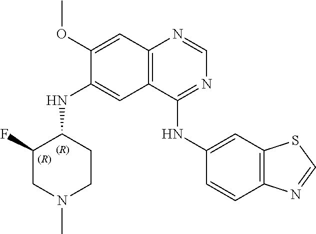 | N4-(benzo[d]thiazol-6-yl)-N6-((3R,4R)-3-fluoro-1-methylpiperidin- 4-yl)-7-methoxyquinazoline-4,6-diamine' LC-MS: (ES, m/z): RT = 1.169 min, LCMS: m/z = 439 [M + 1], ¹H NMR (300 MHz, DMSO-d6) δ 9.43 (s, 1H), 9.30 (s, 1H), 8.72 (dd, J = 5.3, 2.1 Hz, 1H), 8.38 (d, J = 1.6 Hz, 1H), 8.09 (d, J = 8.8 Hz, 1H), 7.94-7.84 (m, 1H), 7.37 (s, 1H), 7.11 (s, 1H), 5.64 (d, J = 9.1 Hz, 1H), 4.95-4.59 (m, 1H), 4.00 (s, 3H), 3.82 (s, 1H), 3.16 (s, 1H), 2.75 (d, J = 11.2 Hz, 1H), 2.28 (s, 3H), 2.13 (dd, J = 26.8, 13.1 Hz, 3H), 1.55 (d, J = 12.3 Hz, 1H). |
| 305 | 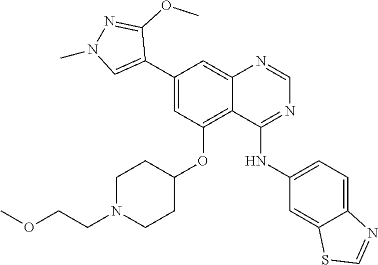 [SMILES: COCCN1CCC(Oc2cc(-c3cn(C)nc3OC)cc3ncnc(Nc4ccc5ncsc5c4)c23)CC1] | N-(7-(3-methoxy-1-methyl-1H-pyrazol-4-yl)-5-((1-(2- methoxyethyl)piperidin-4-yl)oxy)quinazolin-4-yl)benzo[d]thiazol-6- amine LC-MS: (ES, m/z): RT = 3.367 min, LCMS: m/z = 546 [M + 1], ¹H NMR (300 MHz, DMSO-d6) δ 10.27 (s, 1H), 9.32 (s, 1H), 8.91 (d, J = 2.2 Hz, 1H), 8.54 (s, 1H), 8.36 (s, 1H), 8.12 (d, J = 8.8 Hz, 1H), 7.75 (dd, J = 8.8, 2.2 Hz, 1H), 7.65 (d, J = 1.4 Hz, 1H), 7.41 (d, J = 1.6 Hz, 1H), 4.86 (t, J = 4.3 Hz, 1H), 3.98 (s, 3H), 3.78 (s, 3H), 3.44 (t, J = 5.8 Hz, 2H), 3.31-3.15 (m, 3H), 2.86 (d, J = 11.9 Hz, 2H), 2.54 (s, 2H), 2.40 (t, J = 10.5 Hz, 2H), 2.23 (d, J = 13.1 Hz, 2H), 1.95 (d, J = 10.1 Hz, 2H), 1.24 (s, 1H). |
| 306 | 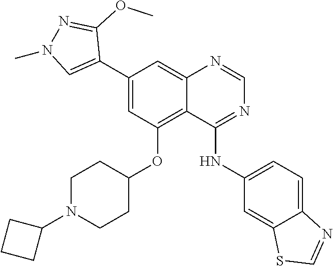 [SMILES: COc1nn(C)cc1-c1cc(OC2CCN(C3CCC3)CC2)c2c(Nc3ccc4ncsc4c3)ncnc2c1] | N-(7-(3-methoxy-1-methyl-1H-pyrazol-4-yl)-5-((1-(oxetan-3- yl)piperidin-4-yl)oxy)quinazolin-4-yl)benzo[d]thiazol-6-amine LC-MS: (ES, m/z): RT = 1.057 min, LCMS: m/z = 544 [M + 1], ¹H NMR (300 MHz, DMSO-d6) δ 10.22 (s, 1H).δ 9.32 (s, 1H), 8.91 (d, J = 2.2 Hz, 1H), 8.53 (s, 1H), 8.33 (s, 1H), 8.11 (d, J = 8.8 Hz, 1H), 7.73 (dd, J = 8.8, 2.2 Hz, 1H), 7.64 (d, J = 1.4 Hz, 1H), 7.40 (d, J = 1.6 Hz, 1H), 4.87 (s, 1H), 4.54 (t, J = 6.5 Hz, 2H), 4.45 (t, J = 6.1 Hz, 2H), 3.97 (s, 3H), 3.77 (s, 3H), 3.44 (q, J = 6.3 Hz, 1H), 2.69 (d, J = 10.0 Hz, 2H), 2.23 (s, 4H), 1.97 (q, J = 10.2, 8.9 Hz, 2H). |
| 307 | 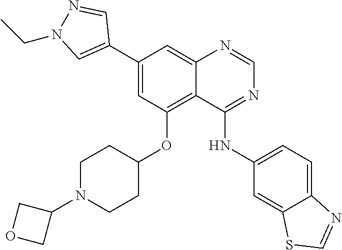 [SMILES: CCn1cc(-c2cc(OC3CCN(C4COC4)CC3)c3c(Nc4ccc5ncsc5c4)ncnc3c2)cn1] | N-(7-(1-ethyl-1H-pyrazol-4-yl)-5-((1-(oxetan-3-yl)piperidin-4- yl)oxy)quinazolin-4-yl)benzo[d]thiazol-6-amine LC-MS: (ES, m/z): RT = 1.032 min, LCMS: m/z = 528 [M + 1], ¹H NMR (300 MHz, DMSO-d6) δ 10.29 (s, 1H), 9.33 (s, 1H), 8.91 (d, J = 2.1 Hz, 1H), 8.54 (d, J = 10.9 Hz, 2H), 8.21-8.09 (m, 2H), 7.76 (dd, J = 8.8, 2.2 Hz, 1H), 7.59 (d, J = 1.4 Hz, 1H), 7.43 (s, 1H), 5.05 (s, 1H), 4.54 (t, J = 6.5 Hz, 2H), 4.45 (t, J = 6.1 Hz, 2H), 4.20 (q, J = 7.3 Hz, 2H), 2.66 (s, 2H), 2.24 (s, 4H), 1.97 (d, J = 9.7 Hz, 2H), 1.45 (t, J = 7.3 Hz, 3H). |
| 308 | 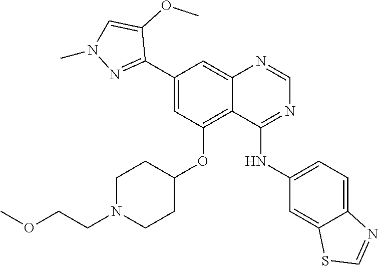 [SMILES: COCCN1CCC(Oc2cc(-c3nn(C)cc3OC)cc3ncnc(Nc4ccc5ncsc5c4)c23)CC1] | N-(7-(4-methoxy-1-methyl-1H-pyrazol-3-yl)-5-((1-(2- methoxyethyl)piperidin-4-yl)oxy)quinazolin-4-yl)benzo[d]thiazol-6- amine LC-MS: (ES, m/z): RT = 1.415 min, LCMS: m/z = 546 [M + 1], ¹H NMR (300 MHz, DMSO-d6) δ 10.29 (s, 1H), 9.33 (s, 1H), 8.91 (s, 1H), 8.58 (s, 1H), 8.13 (d, J = 8.8 Hz, 1H), 7.88 (s, 1H), 7.82-7.68 (m, 2H), 7.66 (s, 1H), 4.83 (s, 1H), 3.87 (d, J = 8.3 Hz, 6H), 3.44- 3.41 (m, 2H), 3.41-3.34 (m, 2H), 3.22 (s, 3H), 2.94-2.76 (m, 2H), 2.45-2.35 (m, 2H), 2.26-2.15 (m, 2H), 2.12-1.95 (m, 2H) |
| 309 | 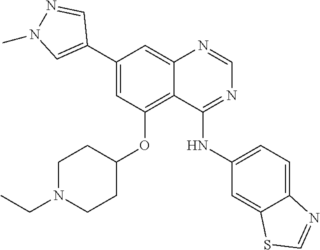 [SMILES: CCN1CCC(Oc2cc(-c3cnn(C)c3)cc3ncnc(Nc4ccc5ncsc5c4)c23)CC1] | N-(5-((1-ethylpiperidin-4-yl)oxy)-7-(1-methyl-1H-pyrazol-4- yl)quinazolin-4-yl)benzo[d]thiazol-6-amine LC-MS: (ES, m/z): RT = 3.106 min, LCMS: m/z = 486 [M + 1], ¹H NMR (300 MHz, DMSO-d6) δ 10.32 (s, 1H), 9.33 (s, 1H), 8.91 (d, J = 2.1 Hz, 1H), 8.56 (s, 1H), 8.48 (s, 1H), 8.20-8.08 (m, 2H), 7.75 (dd, J = 8.8, 2.2 Hz, 1H), 7.57 (d, J = 1.4 Hz, 1H), 7.41 (d, J = 1.5 Hz, 1H), 5.01 (dd, J = 8.5, 4.4 Hz, 1H), 3.92 (s, 3H), 2.81-2.62 (m, 2H), 2.37-2.24 (m, 6H), 1.95 (d, J = 9.7 Hz, 2H), 1.01 (t, J = 7.2 Hz, 3H). |
| 310 | 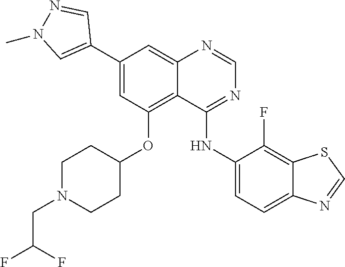 [SMILES: Cn1cc(-c2cc(OC3CCN(CC(F)F)CC3)c3c(Nc4ccc5ncsc5c4F)ncnc3c2)cn1] | N-(5-((1-(2,2-difluoroethyl)piperidin-4-yl)oxy)-7-(1-methyl-1H- pyrazol-4-yl)quinazolin-4-yl)-7-fluorobenzo[d]thiazol-6-amine LC-MS: (ES, m/z): RT = 1.656 min, LCMS: m/z = 540 [M + 1], ¹H- NMR 1H NMR (400 MHz, DMSO-d6) δ 10.09 (s, 1H), 9.48 (d, J = 1.5 Hz, 1H), 8.11 (s, 1H), 8.07-7.98 (m, 2H), 7.90 (s, 1H), 7.81- 7.73 (m, 2H), 7.21 (s, 1H), 4.15 (dt, J = 10.2, 5.1 Hz, 1H), 2.89 (d, J = 10.7 Hz, 2H), 2.24 (s, 3H), 2.10 (s, 2H), 2.02 (s, 4H). |
| 311 | 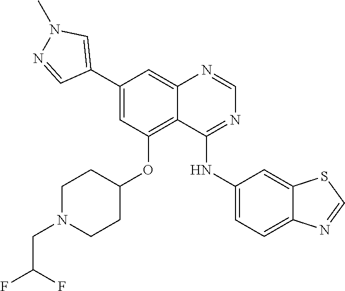 [SMILES: Cn1cc(-c2cc(OC3CCN(CC(F)F)CC3)c3c(Nc4ccc5ncsc5c4)ncnc3c2)cn1] | N-(5-((1-(2,2-difluoroethyl)piperidin-4-yl)oxy)-7-(1-methyl-1H- pyrazol-4-yl)quinazolin-4-yl)benzo[d]thiazol-6-amine LC-MS: (ES, m/z): RT = 1.498 min, LCMS: m/z = 522 [M + 1], ¹H NMR (300 MHz, DMSO-d6) δ 10.28 (s, 1H), 9.33 (s, 1H), 8.91 (d, J = 2.2 Hz, 1H), 8.56 (d, J = 1.3 Hz, 1H), 8.48 (s, 1H), 8.20-8.08 (m, 2H), 7.75 (dd, J = 8.8, 2.3 Hz, 1H), 7.58 (d, J = 1.4 Hz, 1H), 7.41 (s, 1H), 6.39-5.94 (m, 1H), 5.01 (s, 1H), 3.92 (s, 3H), 2.93 (d, J = 11.6 Hz, 2H), 2.79 (td, J = 15.6, 4.2 Hz, 2H), 2.60 (d, J = 10.4 Hz, 2H), 2.25 (d, J = 13.5 Hz, 2H), 1.96 (d, J = 10.9 Hz, 2H). |
| 312 | 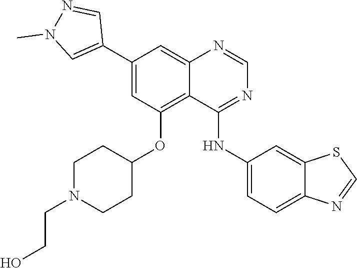 [SMILES: Cn1cc(-c2cc(OC3CCN(CCO)CC3)c3c(Nc4ccc5ncsc5c4)ncnc3c2)cn1] | 2-(4-((4-(benzo[d]thiazol-6-ylamino)-7-(1-methyl-1H-pyrazol-4- yl)quinazolin-5-yl)oxy)piperidin-1-yl)ethan-1-ol LC-MS: (ES, m/z): RT = 2.341 min, LCMS: m/z = 500 [M + 1], ¹H NMR (300 MHz, DMSO-d6) δ 10.32 (s, 1H), 9.33 (s, 1H), 8.92 (d, J = 2.2 Hz, 1H), 8.56 (d, J = 1.3 Hz, 1H), 8.48 (s, 1H), 8.20-8.08 (m, 2H), 7.74 (dd, J = 8.8, 2.2 Hz, 1H), 7.57 (d, J = 1.4 Hz, 1H), 7.41 (s, 1H), 5.01 (s, 1H), 4.40 (t, J = 5.3 Hz, 1H), 3.92 (s, 3H), 3.52 (q, J = 6.0 Hz, 2H), 2.85 (d, J = 11.9 Hz, 2H), 2.49-2.35 (m, 4H), 2.22 (s, 2H), 1.94 (d, J = 10.1 Hz, 2H). |
| 313 | 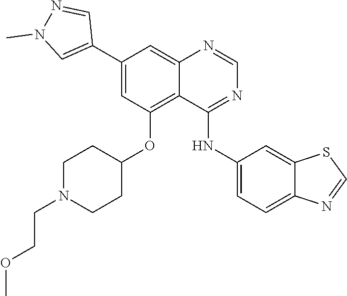 [SMILES: COCCN1CCC(Oc2cc(-c3cnn(C)c3)cc3ncnc(Nc4ccc5ncsc5c4)c23)CC1] | N-(5-((1-(2-methoxyethyl)piperidin-4-yl)oxy)-7-(1-methyl-1H- pyrazol-4-yl)quinazolin-4-yl)benzo[d]thiazol-6-amine LC-MS: (ES, m/z): RT = 1.489 min, LCMS: m/z = 522 [M + 1], ¹H NMR (300 MHz, DMSO-d6) δ 10.28 (s, 1H), 9.33 (s, 1H), 8.91 (d, J = 2.2 Hz, 1H), 8.56 (d, J = 1.3 Hz, 1H), 8.48 (s, 1H), 8.20-8.08 (m, 2H), 7.75 (dd, J = 8.8, 2.3 Hz, 1H), 7.58 (d, J = 1.4 Hz, 1H), 7.41 (s, 1H), 6.39-5.94 (m, 1H), 5.01 (s, 1H), 3.92 (s, 3H), 2.93 (d, J = 11.6 Hz, 2H), 2.79 (td, J = 15.6, 4.2 Hz, 2H), 2.60 (d, J = 10.4 Hz, 2H), 2.25 (d, J = 13.5 Hz, 2H), 1.96 (d, J = 10.9 Hz, 2H). |
| 314 | 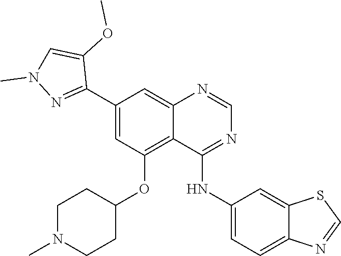 [SMILES: COc1cn(C)nc1-c1cc(OC2CCN(C)CC2)c2c(Nc3ccc4ncsc4c3)ncnc2c1] | N-(7-(4-methoxy-1-methyl-1H-pyrazol-3-yl)-5-((1-methylpiperidin- 4-yl)oxy)quinazolin-4-yl)benzo[d]thiazol-6-amine LC-MS: (ES, m/z): RT = 2.289 min, LCMS: m/z = 502 [M + 1], ¹H NMR (300 MHz, DMSO-d6) δ 10.27 (s, 1H), 9.32 (s, 1H), 8.94- 8.88 (m, 1H), 8.55 (d, J = 1.1 Hz, 1H), 8.36 (s, 1H), 8.12 (d, J = 8.8 Hz, 1H), 7.75 (dd, J = 8.9, 2.2 Hz, 1H), 7.66 (d, J = 1.4 Hz, 1H), 7.43 (d, J = 1.6 Hz, 1H), 4.88 (s, 1H), 3.98 (s, 3H), 3.79 (s, 3H), 2.73 (d, J = 2.6 Hz, 3H), 2.28 (s, 2H), 2.23 (s, 4H), 2.00 (s, 2H). |
| 315 | 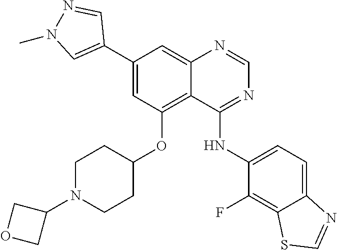 [SMILES: Cn1cc(-c2cc(OC3CCN(C4COC4)CC3)c3c(Nc4ccc5ncsc5c4F)ncnc3c2)cn1] | 7-fluoro-N-(7-(1-methyl-1H-pyrazol-4-yl)-5-((1-(oxetan-3- yl)piperidin-4-yl)oxy)quinazolin-4-yl)benzo[d]thiazol-6-amine LC-MS: (ES, m/z): RT = 1.281 min, LCMS: m/z = 532.4, [M + 1], ¹H NMR (400 MHz, DMSO-d6) δ 10.11 (s, 1H), 9.35 (d, J = 1.2 Hz, 1H), 8.64 (t, J = 8.4 Hz, 1H), 8.46 (s, 1H), 8.40 (s, 1H), 8.10 (s, 1H), 7.96 (d, J = 8.8 Hz, 1H), 7.52 (d, J = 1.2 Hz, 1H), 7.37 (s, 1H), 4.97 (s, 1H), 4.48-4.37 (m, 4H), 3.84 (s, 3H), 2.63 (d, J = 11.6 Hz, 2H), 2.16-1.83 (m, 6H). |
| 316 | 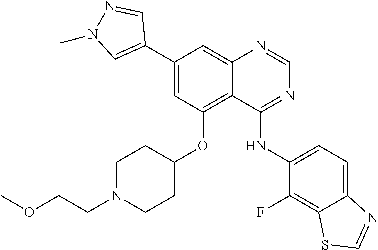 [SMILES: COCCN1CCC(Oc2cc(-c3cnn(C)c3)cc3ncnc(Nc4ccc5ncsc5c4F)c23)CC1] | 7-fluoro-N-(5-((1-(2-methoxyethyl)piperidin-4-yl)oxy)-7-(1-methyl- 1H-pyrazol-4-yl)quinazolin-4-yl)benzo[d]thiazol-6-amine LC-MS: (ES, m/z): RT = 1.388 min, LCMS: m/z = 534, [M + 1], ¹H NMR (400 MHz, DMSO-d6) δ 10.18 (s, 1H), 9.42 (d, J = 1.6 Hz, 1H), 8.72 (t, J = 8.5 Hz, 1H), 8.54 (s, 1H), 8.48 (s, 1H), 8.16 (d, J = 13.2 Hz, 1H), 8.03 (d, J = 8.8 Hz, 1H), 7.59 (d, J = 1.2 Hz, 1H), 7.44 (s, 1H), 5.03 (s, 1H), 3.92 (s, 3H), 3.47 (s, 2H), 3.23 (s, 3H), 2.91 (s, 2H), 2.57 (s, 3H), 2.20 (s, 2H), 1.93 (s, 2H). |
| 317 | 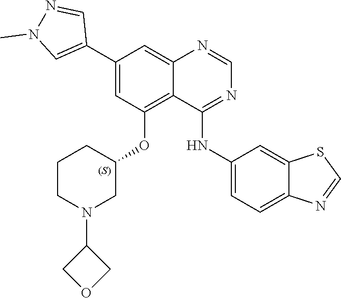 | (S)-N-(7-(1-methyl-1H-pyrazol-4-yl)-5-((1-(oxetan-3-yl)piperidin-3- yl)oxy)quinazolin-4-yl)benzo[d]thiazol-6-amine LC-MS: (ES, m/z): RT = 1.310 min, m/z = 445 [M + 1], ¹H NMR (400 MHz, DMSO-d6) δ 10.70 (s, 1H), 9.31 (s, 1H), 8.85 (d, J = 2.0 Hz, 1H), 8.50 (d, J = 26.5 Hz, 2H), 8.17-7.72 (m, 3H), 7.56 (d, J = 1.4 Hz, 1H), 7.44 (d, J = 1.6 Hz, 1H), 5.32 (s, 1H), 4.59-4.39 (m, 4H), 3.91 (s, 3H), 3.54 (p, J = 6.4 Hz, 1H), 3.08 (d, J = 12.0 Hz, 1H), 2.80 (d, J = 10.8 Hz, 1H), 2.36 (d, J = 12.4 Hz, 1H), 2.10-1.90 (m, 5H), 1.80-1.60 (m, 1H), 1.59-1.50 (m, 2H). |
| 318 | 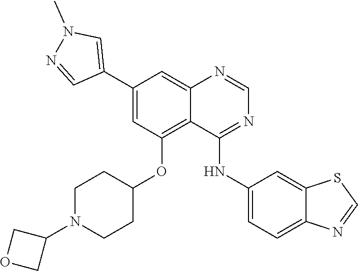 [SMILES: Cn1cc(-c2cc(OC3CCN(C4COC4)CC3)c3c(Nc4ccc5ncsc5c4)ncnc3c2)cn1] | N-(7-(1-methyl-1H-pyrazol-4-yl)-5-((1-(oxetan-3-yl)piperidin-4- yl)oxy)quinazolin-4-yl)benzo[d]thiazol-6-amine LC-MS: (ES, m/z): RT = 2.024 min, LCMS: m/z = 514 [M + 1], ¹H NMR (300 MHz, DMSO-d6) δ 10.28 (s, 1H), 9.33 (s, 1H), 8.91 (d, J = 2.2 Hz, 1H), 8.56 (d, J = 1.3 Hz, 1H), 8.47 (s, 1H), 8.19-8.08 (m, 2H), 7.76 (dd, J = 8.8, 2.2 Hz, 1H), 7.58 (d, J = 1.4 Hz, 1H), 7.41 (s, 1H), 5.04 (s, 1H), 4.54 (t, J = 6.4 Hz, 2H), 4.46 (t, J = 6.1 Hz, 2H), 3.92 (s, 3H), 3.49-3.39 (m, 1H), 2.69 (s, 2H), 2.23 (t, J = 10.4 Hz, 4H), 1.98 (d, J = 10.2 Hz, 2H). |
| 319 | 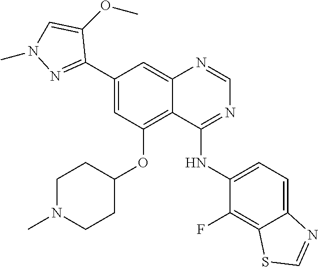 [SMILES: COc1cn(C)nc1-c1cc(OC2CCN(C)CC2)c2c(Nc3ccc4ncsc4c3F)ncnc2c1] | 7-fluoro-N-(7-(4-methoxy-1-methyl-1H-pyrazol-3-yl)-5-((1- methylpiperidin-4-yl)oxy)quinazolin-4-yl)benzo[d]thiazol-6-amine LC-MS: (ES, m/z): RT = 1.482 min, LCMS: m/z = 520 [M + 1]. ¹H NMR (300 MHz, DMSO-d6) δ 10.17 (s, 1H), 9.41 (d, J = 1.6 Hz, 1H), 8.69 (t, J = 8.4 Hz, 1H), 8.54 (s, 1H), 8.02 (d, J = 8.9 Hz, 1H), 7.88 (d, J = 1.3 Hz, 1H), 7.73-7.64 (m, 2H), 4.81 (s, 1H), 3.85 (d, J = 8.3 Hz, 6H), 2.81-2.65 (m, 2H), 2.34-2.12 (m, 7H), 1.93 (m, 2H). |
| 320 | 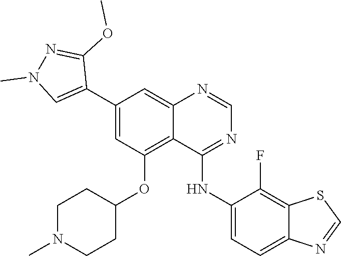 [SMILES: COc1nn(C)cc1-c1cc(OC2CCN(C)CC2)c2c(Nc3ccc4ncsc4c3F)ncnc2c1] | 7-fluoro-N-(7-(3-methoxy-1-methyl-1H-pyrazol-4-yl)-5-((1- methylpiperidin-4-yl)oxy)quinazolin-4-yl)benzo[d]thiazol-6-amine LC-MS: (ES, m/z): RT = 2.256 min, LCMS: m/z = 520 [M + 1], ¹H NMR (400 MHz, DMSO-d6) δ 10.17 (s, 1H), 9.42 (s, 1H), 8.74 (t, J = 8.6 Hz, 1H), 8.52 (s, 1H), 8.36 (s, 1H), 8.03 (d, J = 9.0 Hz, 1H), 7.67 (s, 1H), 7.45 (s, 1H), 4.88 (s, 1H), 3.98 (s, 3H), 3.78 (s, 3H), 2.75 (s, 2H), 2.24 (d, J = 20.6 Hz, 7H), 1.92 (d, J = 11.0 Hz, 2H). |
| 321 322 | 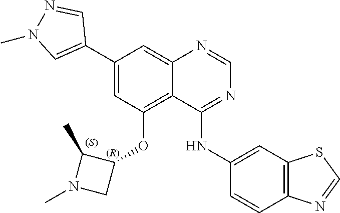 or 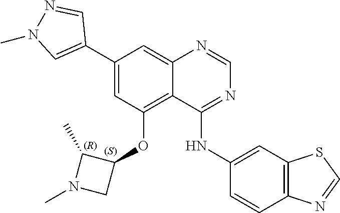 | N-(5-(((2S,3R)-1,2-dimethylazetidin-3-yl)oxy)-7-(1-methyl-1H- pyrazol-4-yl)quinazolin-4-yl)benzo[d]thiazol-6-amine OR N-(5- (((2R,3S)-1,2-dimethylazetidin-3-yl)oxy)-7-(1-methyl-1H-pyrazol-4- yl)quinazolin-4-yl)benzo[d]thiazol-6-amine First Eluting Isomer: LC-MS: (ES, m/z): RT = 2.489 min, LCMS: m/z = 458 [M + 1], chiral-HPLC: R = 2.144, ¹H NMR (400 MHz, DMSO-d6) δ 9.95 (s, 1H), 9.33 (s, 1H), 8.92 (d, J = 2.1 Hz, 1H), 8.58 (s, 1H), 8.45 (s, 1H), 8.13 (d, J = 8.7 Hz, 2H), 7.80 (d, J = 8.8 Hz, 1H), 7.63 (d, J = 1.4 Hz, 1H), 7.04 (s, 1H), 5.11 (s, 1H), 4.49 (s, 1H), 3.92 (s, 3H), 2.81-2.61 (m, 2H), 1.58 (s, 3H). Second Eluting Isomer:-MS: (ES, m/z): RT = 2.486 min, m/z = 458 [M + 1], chiral-HPLC: R = 2.638, ¹H NMR (400 MHz, DMSO-d6) δ 9.97 (s, 1H), 9.33 (s, 1H), 8.92 (d, J = 2.2 Hz, 1H), 8.58 (s, 1H), 8.45 (s, 1H), 8.13 (d, J = 8.3 Hz, 2H), 7.79 (s, 1H), 7.62 (d, J = 1.4 Hz, 1H), 7.05 (s, 1H), 5.07 (s, 1H), 4.41 (brs, 1H), 3.92 (s, 3H), 3.30 (s, 3H), 2.67 (s, 2H), 1.56 (s, 3H). |
| 325 326 | 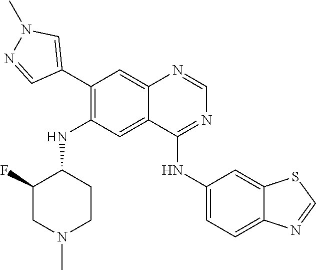 [SMILES: CN1CC[C@@H](Nc2cc3c(Nc4ccc5ncsc5c4)ncnc3cc2-c2cnn(C)c2)[C@H](F)C1] or 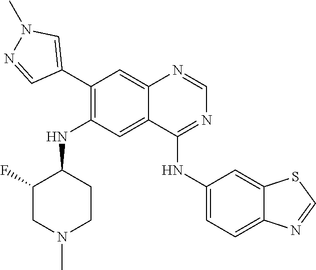 [SMILES: CN1CC[C@H](Nc2cc3c(Nc4ccc5ncsc5c4)ncnc3cc2-c2cnn(C)c2)[C@@H](F)C1] | N4-(benzo[d]thiazol-6-yl)-N6 (3S,4S)-3-fluoro-1-methylpiperidin-4- yl)-7-(1-methyl-1H-pyrazol-4-yl)quinazoline-4,6-diamine OR N4- (benzo[d]thiazol-6-yl)-N6-(3R,4R)-3-fluoro-1-methylpiperidin-4- yl)-7-(1-methyl-1H-pyrazol-4-yl)quinazoline-4,6-diamine First Eluting Isomer: LC-MS: (ES, m/z): RT = 0.990 min, LCMS: m/z = 489[M + 1], ¹H NMR (300 MHz, DMSO-d6) δ 9.62 (s, 1H), 9.32 (s, 1H), 8.74 (d, J = 2.0 Hz, 1H), 8.39 (s, 1H), 8.12 (d, J = 9.0 Hz, 2H), 7.91 (dd, J = 8.8, 2.1 Hz, 1H), 7.83 (d, J = 0.8 Hz, 1H), 7.57 (s, 1H), 7.48 (s, 1H), 5.12 (d, J = 8.7 Hz, 1H), 4.84 (s, 1H), 3.95 (s, 3H), 3.89 (s, 1H), 3.10 (s, 1H), 2.68 (s, 1H), 2.28 (s, 3H), 2.18 (s, 3H), 1.55 (d, J = 12.3 Hz, 1H). Second Eluting Isomer: LC-MS: (ES, m/z): RT = 0.990 min, LCMS: m/z = 489[M + 1], ¹H NMR (300 MHz, DMSO-d6) δ 9.62 (s, 1H), 9.32 (s, 1H), 8.74 (d, J = 2.0 Hz, 1H), 8.39 (s, 1H), 8.12 (d, J = 9.0 Hz, 2H), 7.91 (dd, J = 8.8, 2.1 Hz, 2H), 7.57 (s, 1H), 7.48 (s, 1H), 5.12 (d, J = 8.7 Hz, 1H), 4.84 (s, 1H), 3.95 (s, 4H), 3.10 (s, 1H), 2.68 (s, 1H), 2.28 (s, 3H), 2.18 (s, 3H), 1.55 (d, J = 12.3 Hz, 1H). |
| 327 328 | 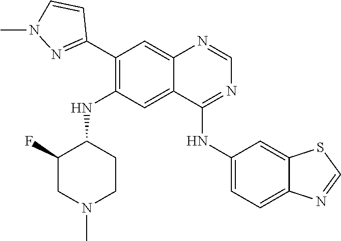 [SMILES: CN1CC[C@@H](Nc2cc3c(Nc4ccc5ncsc5c4)ncnc3cc2-c2ccn(C)n2)[C@H](F)C1] 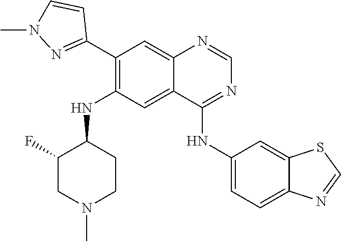 [SMILES: CN1CC[C@H](Nc2cc3c(Nc4ccc5ncsc5c4)ncnc3cc2-c2ccn(C)n2)[C@@H](F)C1] | N4-(benzo[d]thiazol-6-yl)-N6-((3R,4R)-3-fluoro-1-methylpiperidin- 4-yl)-7-(1-methyl-1H-pyrazol-3-yl)quinazoline-4,6-diamine, OR N4- (benzo[d]thiazol-6-yl)-N6-((3S,4S)-3-fluoro-1-methylpiperidin-4- yl)-7-(1-methyl-1H-pyrazol-3-yl)quinazoline-4,6-diamine First Eluting Isomer: LC-MS: (ES, m/z): RT = 3.067 min, LCMS: m/z = 489[M + 1], ¹H NMR (300 MHz, DMSO-d6) δ 9.71 (s, 1H), 9.33 (s, 1H), 8.72 (d, J = 1.9 Hz, 1H), 8.41 (s, 1H), 8.17-8.07 (m, 3H), 7.96-7.85 (m, 2H), 7.60 (s, 1H), 7.08 (s, 1H), 4.57-4.67 (m, 1H),4.01 (s, 4H), 2.91 (s, 1H), 2.61 (s, 2H), 2.21 (s, 5H)1.56 (t, J = 7.0 Hz, 1H). Second Eluting Isomer: LC-MS: (ES, m/z): RT = 3.067 min, LCMS: m/z = 489[M + 1], ¹H NMR (300 MHz, DMSO-d6) δ 9.71 (s, 1H), 9.33 (s, 1H), 8.72 (d, J = 1.9 Hz, 1H), 8.41 (s, 1H), 8.17-8.07 (m,3H), 7.96-7.85 (m, 2H), 7.60 (s, 1H), 7.08 (s, 1H), 4.61 (s, 2H),4.01 (s,6H), 3.01 (s, 3H), 2.31 (s, 5H), 1.56 (t, J = 7.0 Hz, 1H). 1.16 (t, J = 7.0 Hz, 3H). |
| 329 | 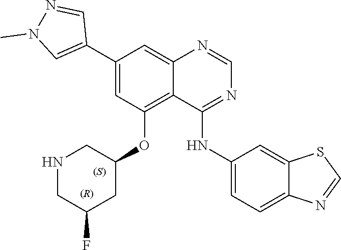 cis-racemic | cis-N-(5-(((3S,5R)-5-fluoropiperidin-3-yl)oxy)-7-(1-methyl-1H- pyrazol-4-yl)quinazolin-4-yl)benzo[d]thiazol-6-amine LC-MS: (ES, m/z): RT = 0.843 min, LCMS: m/z = 476 [M + 1]. ¹H NMR (400 MHz, DMSO-d6) δ 10.70 (d, J = 1.5 Hz, 1H), 9.30 (s, 1H), 8.83 (d, J = 1.9 Hz, 1H), 8.47 (d, J = 16.0 Hz, 2H), 8.14 (s, 1H), 8.08-7.98 (m, 2H), 7.52 (d, J = 1.4 Hz, 1H), 7.36 (d, J = 1.6 Hz, 1H), 5.13 (s, 1H), 4.79 (d, J = 47.6 Hz, 1H), 3.91 (s, 3H), 3.47-3.08 (m, 2H), 3.05-2.80 (m, 2H), 2.40 (d, J = 14.6 Hz, 2H), 2.18 (dd, J = 40.0, 15.3 Hz, 1H). |
| 331 | 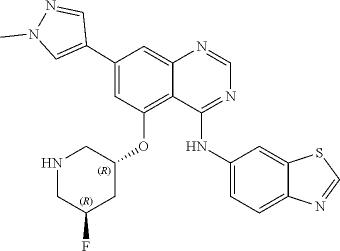 trans racemic | trans-N-(5-(((3R,5R)-5-fluoropiperidin-3-yl)oxy)-7-(1-methyl-1H- pyrazol-4-yl)quinazolin-4-yl)benzo[d]thiazol-6-amine and N-(5- (((3S,5S)-5-fluoropiperidin-3-yl)oxy)-7-(1-methyl-1H-pyrazol-4- yl)quinazolin-4-yl)benzo[d]thiazol-6-amine LC-MS: (ES, m/z): RT = 1.168 min, LCMS: m/z = 476 [M + 1]. ¹H NMR (400 MHz, DMSO-d6) δ 10.53 (s, 1H), 9.31 (s, 1H), 8.90 (d, J = 2.1 Hz, 1H), 8.55 (s, 1H), 8.47 (s, 1H), 8.17 (d, J = 0.8 Hz, 1H), 8.08 (d, J = 8.8 Hz, 1H), 7.97 (dd, J = 8.9, 2.2 Hz, 1H), 7.58 (d, J = 1.5 Hz, 1H), 7.45 (d, J = 1.7 Hz, 1H), 5.28 (s, 1H), 4.86 (ddt, J = 48.5, 8.2, 4.0 Hz, 1H), 3.92 (s, 3H), 3.20 (t, J = 13.9 Hz, 1H), 3.07 (s, 2H), 2.97 (s, 1H), 2.78 (dt, J = 11.5, 7.5 Hz, 1H), 2.11 (q, J = 11.3 Hz, 1H). |
| 332 333 | 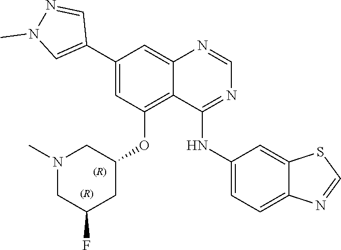 or 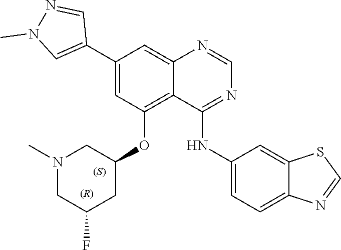 | N-(5-(((3R,5R)-5-fluoropiperidin-3-yl)oxy)-7-(1-methyl-1H-pyrazol- 4-yl)quinazolin-4-yl)benzo[d]thiazol-6-amine OR N-(5-(((3S,5S)- 5-fluoropiperidin-3-yl)oxy)-7-(1-methyl-1H-pyrazol-4-yl)quinazolin- 4-yl)benzo[d]thiazol-6-amine First Eluting Isomer: LC-MS: (ES, m/z): RT = 1.07 min, LCMS: m/z = 490 [M + 1]. Chiral-HPLC R = 1.912, 1H NMR (400 MHz, DMSO- d6) δ 10.55 (s, 1H), 9.31 (s, 1H), 8.90 (d, J = 2.1 Hz, 1H), 8.56 (s, 1H), 8.47 (s, 1H), 8.18-8.08 (m, 2H), 7.92 (dd, J = 8.9, 2.2 Hz, 1H), 7.58 (d, J = 1.4 Hz, 1H), 7.45 (d, J = 1.6 Hz, 1H), 5.43 (d, J = 4.5 Hz, 1H), 4.91 (dt, J = 48.1, 4.9 Hz, 1H), 3.92 (s, 3H), 3.25-3.02 (m, 2H), 2.61 (s, 1H), 2.55-2.54 (m, 1H),2.40 (s, 3H), 2.24 (td, J = 9.9, 5.9 Hz, 1H), 1.82 (q, J = 12.2 Hz, 1H). Second Eluting Isomer: LC-MS: (ES, m/z): RT = 0.689 min, LCMS: m/z = 490 [M + 1]. Chiral-HPLC R = 2.460, 1H NMR (400 MHz, DMSO-d6) δ 10.55 (s, 1H), 9.31 (s, 1H), 8.90 (d, J = 2.1 Hz, 1H), 8.56 (s, 1H), 8.47 (s, 1H), 8.18-8.08 (m, 2H), 7.92 (dd, J = 8.9, 2.1 Hz, 1H), 7.58 (d, J = 1.4 Hz, 1H), 7.45 (d, J = 1.5 Hz, 1H), 5.43 (d, J = 4.4 Hz, 1H), 4.91 (dtd, J = 52.7, 9.7, 4.6 Hz, 1H), 3.92 (s, 3H), 3.26-2.95 (m, 2H), 2.61 (s, 1H), 2.48 (d, J = 1.8 Hz, 0H), 2.40 (s, 3H), 2.24 (td, J = 9.9, 5.9 Hz, 1H), 1.89-1.75 (m, 1H).. |
| 334 335 | 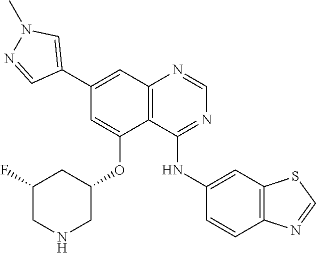 [SMILES: Cn1cc(-c2cc(O[C@@H]3CNC[C@H](F)C3)c3c(Nc4ccc5ncsc5c4)ncnc3c2)cn1] or 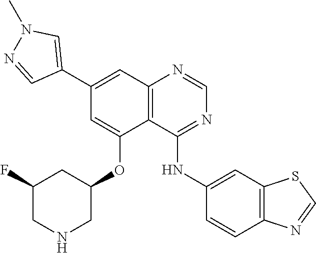 [SMILES: Cn1cc(-c2cc(O[C@H]3CNC[C@@H](F)C3)c3c(Nc4ccc5ncsc5c4)ncnc3c2)cn1] | N-(5-(((3S,5R)-5-fluoropiperidin-3-yl)oxy)-7-(1-methyl-1H-pyrazol- 4-yl)quinazolin-4-yl)benzo[d]thiazol-6-amine, OR N-(5-(((3R,5S)-5- fluoropiperidin-3-yl)oxy)-7-(1-methyl-1H-pyrazol-4-yl)quinazolin-4- yl)benzo[d]thiazol-6-amine First Eluting Isomer: LC-MS: (ES, m/z): RT = 0.867 min, LCMS: m/z = 476 [M + 1], Chiral-HPLC (ES): RT = 1.917, ¹H NMR (400 MHz, DMSO-d6) δ 10.73-10.68 (m, 1H), 9.30 (s, 1H), 8.83 (d, J = 1.9 Hz, 1H), 8.49 (s, 1H), 8.45 (s, 1H), 8.15 (d, J = 0.8 Hz, 1H), 8.09- 7.98 (m, 2H), 7.53 (d, J = 1.5 Hz, 1H), 7.37 (d, J = 1.6 Hz, 1H), 5.14 (s, 1H), 4.80 (d, J = 47.5 Hz, 1H), 3.91 (s, 3H), 3.32-3.12 (m, 2H), 3.06-2.84 (m, 2H), 2.72 (s, 1H), 2.43 (t, J = 13.5 Hz, 1H), 2.19 (dd, J = 40.2, 15.2 Hz, 1H). Second Eluting Isomer: LC-MS: (ES, m/z): RT = 1.064 min, LCMS: m/z = 476 [M + 1], Chiral-HPLC (ES): RT = 2.655, 1H NMR (400 MHz, DMSO-d6) δ 10.71 (s, 1H), 9.30 (s, 1H), 8.83 (d, J = 1.9 Hz, 1H), 8.47 (d, J = 16.5 Hz, 2H), 8.15 (s, 1H), 8.09-7.98 (m, 2H), 7.53 (d, J = 1.5 Hz, 1H), 7.37 (d, J = 1.4 Hz, 1H), 5.14 (s, 1H), 4.80 (d, J = 47.6 Hz, 1H), 3.91 (s, 3H), 3.28 (s, 0H), 3.22 (d, J = 20.2 Hz, 1H), 3.33-3.17 (m, 3H), 3.16 (s, 0H), 3.06-2.83 (m, 2H), 2.41 (d, J = 13.8 Hz, 1H), 2.18 (dd, J = 40.1, 15.4 Hz, 1H). |
| 336 337 | 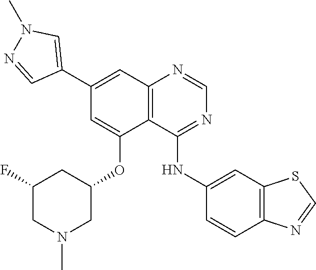 [SMILES: CN1C[C@H](F)C[C@H](Oc2cc(-c3cnn(C)c3)cc3ncnc(Nc4ccc5ncsc5c4)c23)C1] or 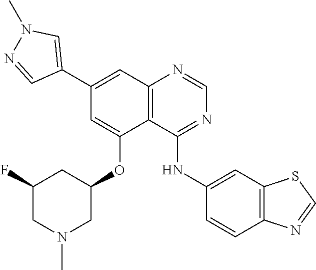 [SMILES: CN1C[C@@H](F)C[C@@H](Oc2cc(-c3cnn(C)c3)cc3ncnc(Nc4ccc5ncsc5c4)c23)C1] | N-(5-(((3S,5R)-5-fluoro-1-methylpiperidin-3-yl)oxy)-7-(1-methyl- 1H-pyrazol-4-yl)quinazolin-4-yl)benzo[d]thiazol-6-amine, OR N-(5- (((3R,5S)-5-fluoro-1-methylpiperidin-3-yl)oxy)-7-(1-methyl-1H- pyrazol-4-yl)quinazolin-4-yl)benzo[d]thiazol-6-amine First Eluting Isomer: LC-MS: (ES, m/z): RT = 0.806 min, LCMS: m/z = 490 [M + 1], Chiral-HPLC (ES): RT = 4.405, ¹H NMR (400 MHz, DMSO-d6) δ 10.81 (s, 1H), 9.27 (s, 1H), 8.88 (d, J = 2.0 Hz, 1H), 8.51 (s, 1H), 8.43 (s, 1H), 8.15-8.05 (m, 2H), 7.99 (dd, J = 8.9, 2.1 Hz, 1H), 7.51 (d, J = 1.4 Hz, 1H), 7.38 (d, J = 1.6 Hz, 1H), 5.30 (s, 1H), 4.90 (d, J = 47.0 Hz, 1H), 3.89 (s, 3H), 3.23 (t, J = 13.5 Hz, 2H), 2.56-2.26 (m, 6H), 2.02-1.80 (m, 1H), 1.21 (s, 1H). Second Eluting Isomer: LC-MS: (ES, m/z): RT = 0.821 min, LCMS: m/z = 490 [M + 1], Chiral-HPLC (ES): RT = 8.810, ¹H NMR (400 MHz, DMSO-d6) δ 10.81 (s, 1H), 9.27 (s, 1H), 8.88 (d, J = 2.0 Hz, 1H), 8.51 (s, 1H), 8.43 (s, 1H), 8.13 (d, J = 0.7 Hz, 1H), 8.07 (d, J = 8.9 Hz, 1H), 7.99 (dd, J = 8.9, 2.1 Hz, 1H), 7.51 (d, J = 1.5 Hz, 1H), 7.38 (d, J = 1.6 Hz, 1H), 5.30 (s, 1H), 4.90 (d, J = 47.0 Hz, 1H), 3.89 (s, 3H), 3.33-3.23 (m, 2H), 2.53-2.34 (m, 6H), 2.15-1.64 (m, 1H). |
| 339 | 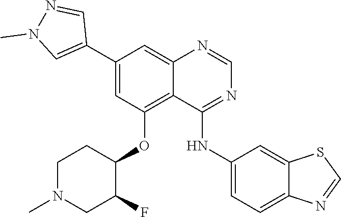 [SMILES: CN1CC[C@@H](Oc2cc(-c3cnn(C)c3)cc3ncnc(Nc4ccc5ncsc5c4)c23)[C@@H](F)C1] cis racemate | cis-N-(5-(((3S,4R)-3-fluoro-1-methylpiperidin-4-yl)oxy)-7-(1- methyl-1H-pyrazol-4-yl)quinazolin-4-yl)benzo[d]thiazol-6-amine LC-MS: (ES, m/z): RT = 1.172 min, LCMS: m/z = 490 [M + 1]. ¹H NMR (300 MHz, DMSO-d6) δ 10.38 (s, 1H), 9.32 (s, 1H), 8.98 (d, J = 2.1 Hz, 1H), 8.59 (s, 1H), 8.46 (s, 1H), 8.18 (d, J = 0.8 Hz, 1H), 8.11 (d, J = 8.8 Hz, 1H), 7.72 (dd, J = 8.9, 2.2 Hz, 1H), 7.61 (d, J = 1.4 Hz, 1H), 7.51 (d, J = 1.6 Hz, 1H), 5.24 (d, J = 50.4 Hz, 2H), 3.92 (s, 3H), 2.86 (d, J = 11.3 Hz, 1H), 2.35 (d, J = 17.4 Hz, 1H), 2.27 (s, 3H), 2.11 (d, J = 16.8 Hz, 2H). |
| 340 341 | 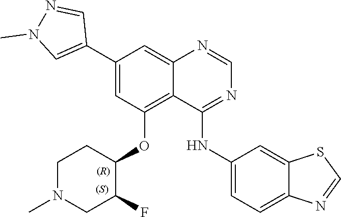 or 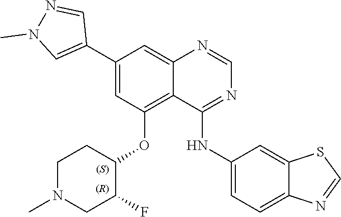 | N-(5-(((3S,4R)-3-fluoro-1-methylpiperidin-4-yl)oxy)-7-(1-methyl- 1H-pyrazol-4-yl)quinazolin-4-yl)benzo[d]thiazol-6-amine OR N- (5-(((3R,4S)-3-fluoro-1-methylpiperidin-4-yl)oxy)-7-(1-methyl-1H- pyrazol-4-yl)quinazolin-4-yl)benzo[d]thiazol-6-amine First Eluting Isomer: LC-MS: (ES, m/z): RT = 1.286 min, LCMS: m/z = 490 [M + 1]. ¹H NMR (300 MHz, DMSO-d6) δ 10.38 (s, 1H), 9.32 (s, 1H), 8.98 (d, J = 2.1 Hz, 1H), 8.59 (s, 1H), 8.46 (s, 1H), 8.18 (d, J = 0.8 Hz, 1H), 8.11 (d, J = 8.8 Hz, 1H), 7.72 (dd, J = 8.9, 2.2 Hz, 1H), 7.61 (d, J = 1.4 Hz, 1H), 7.51 (d, J = 1.6 Hz, 1H), 5.24 (d, J = 50.4 Hz, 2H), 3.92 (s, 3H), 2.86 (d, J = 11.3 Hz, 1H), 2.35 (d, J = 17.4 Hz, 1H), 2.27 (s, 3H), 2.11 (d, J = 16.8 Hz, 2H). Second Eluting Isomer: LC-MS: (ES, m/z): RT = 1.286 min, LCMS: m/z = 490 [M + 1]. ¹H NMR (300 MHz, DMSO-d6) δ 10.38 (s, 1H), 9.32 (s, 1H), 8.98 (d, J = 2.1 Hz, 1H), 8.59 (s, 1H), 8.46 (s, 1H), 8.18 (d, J = 0.8 Hz, 1H), 8.11 (d, J = 8.8 Hz, 1H), 7.72 (dd, J = 8.9, 2.2 Hz, 1H), 7.61 (d, J = 1.4 Hz, 1H), 7.51 (d, J = 1.6 Hz, 1H), 5.24 (d, J = 50.4 Hz, 2H), 3.92 (s, 3H), 2.86 (d, J = 11.3 Hz, 1H), 2.35 (d, J = 17.4 Hz, 1H), 2.27 (s, 3H), 2.11 (d, J = 16.8 Hz, 2H). |
| 343 | 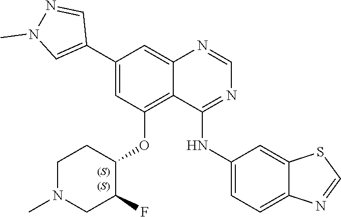 trans racemic | trans-N-(5-(((3S,4S)-3-fluoro-1-methylpiperidin-4-yl)oxy)-7-(1- methyl-1H-pyrazol-4-yl)quinazolin-4-yl)benzo[d]thiazol-6-amine LC-MS: (ES, m/z): RT = 2.336 min, LCMS: m/z = 490 [M + 1]. ¹H NMR (300 MHz, DMSO-d6) δ 10.18 (s, 1H), 9.32 (s, 1H), 8.89 (d, J = 2.1 Hz, 1H), 8.57 (s, 1H), 8.47 (s, 1H), 8.17 (d, J = 0.8 Hz, 1H), 8.11 (d, J = 8.8 Hz, 1H), 7.83 (dd, J = 8.9, 2.2 Hz, 1H), 7.60 (d, J = 1.4 Hz, 1H), 7.47 (d, J = 1.6 Hz, 1H), 5.32-4.95 (m, 2H), 3.92 (s, 3H), 2.79 (d, J = 11.9 Hz, 1H), 2.30 (s, 6H), 2.01-1.83 (m, 1H). |
| 344 345 | 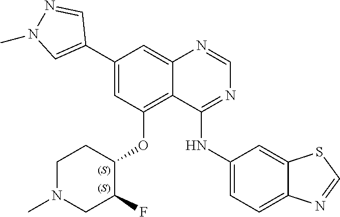 or 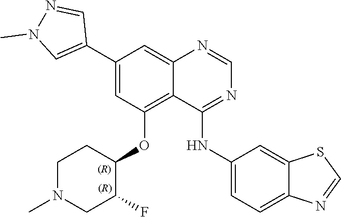 | N-(5-(((3S,4S)-3-fluoro-1-methylpiperidin-4-yl)oxy)-7-(1-methyl- 1H-pyrazol-4-yl)quinazolin-4-yl)benzo[d]thiazol-6-amine OR N- (5-(((3R,4R)-3-fluoro-1-methylpiperidin-4-yl)oxy)-7-(1-methyl-1H- pyrazol-4-yl)quinazolin-4-yl)benzo[d]thiazol-6-amine First Eluting Isomer: LC-MS: (ES, m/z): RT = 1.319 min, LCMS: m/z = 490 [M + 1]. ¹H NMR (300 MHz, DMSO-d6) δ 10.18 (s, 1H), 9.32 (s, 1H), 8.89 (d, J = 2.1 Hz, 1H), 8.57 (s, 1H), 8.47 (s, 1H), 8.17 (d, J = 0.8 Hz, 1H), 8.11 (d, J = 8.8 Hz, 1H), 7.83 (dd, J = 8.9, 2.2 Hz, 1H), 7.60 (d, J = 1.4 Hz, 1H), 7.47 (d, J = 1.6 Hz, 1H), 5.32- 4.95 (m, 2H), 3.92 (s, 3H), 2.79 (d, J = 11.9 Hz, 1H), 2.30 (s, 6H), 2.01-1.83 (m, 1H). Second Eluting Isomer: LC-MS: (ES, m/z): RT = 1.319 min, LCMS: m/z = 490 [M + 1]. ¹H NMR (300 MHz, DMSO-d6) δ 10.18 (s, 1H), 9.32 (s, 1H), 8.89 (d, J = 2.1 Hz, 1H), 8.57 (s, 1H), 8.47 (s, 1H), 8.17 (d, J = 0.8 Hz, 1H), 8.11 (d, J = 8.8 Hz, 1H), 7.83 (dd, J = 8.9, 2.2 Hz, 1H), 7.60 (d, J = 1.4 Hz, 1H), 7.47 (d, J = 1.6 Hz, 1H), 5.32- 4.95 (m, 2H), 3.92 (s, 3H), 2.79 (d, J = 11.9 Hz, 1H), 2.30 (s, 6H), 2.01-1.83 (m, 1H). |
| 346 347 | 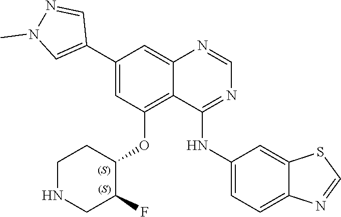 or 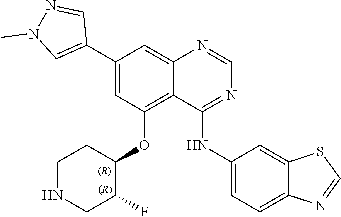 | N-(5-(((3S,4S)-3-fluoropiperidin-4-yl)oxy)-7-(1-methyl-1H-pyrazol- 4-yl)quinazolin-4-yl)benzo[d]thiazol-6-amine OR N-(5-(((3R,4R)- 3-fluoropiperidin-4-yl)oxy)-7-(1-methyl-1H-pyrazol-4-yl)quinazolin- 4-yl)benzo[d]thiazol-6-amine First Eluting Isomer: LC-MS: (ES, m/z): RT = 1.172 min, LCMS: m/z = 476 [M + 1]. ¹H NMR (400 MHz, DMSO-d6) δ 10.27 (s, 1H), 9.33 (s, 1H), 8.92 (d, J = 2.1 Hz, 1H), 8.58 (s, 1H), 8.47 (s, 1H), 8.21- 8.07 (m, 2H), 7.79 (dd, J = 8.8, 2.2 Hz, 1H), 7.60 (d, J = 1.4 Hz, 1H), 7.49 (d, J = 1.6 Hz, 1H), 5.28-4.83 (m, 2H), 3.92 (s, 3H), 3.38 (t, J = 5.6 Hz, 1H), 3.02-2.88 (m, 1H), 2.70 (ddd, J = 12.6, 10.0, 5.5 Hz, 2H), 2.43-2.31 (m, 1H), 1.75 (qd, J = 11.9, 4.4 Hz, 1H). Second Eluting Isomer: LC-MS: (ES, m/z): RT = 1.189 min, LCMS: m/z = 476 [M + 1]. ¹H NMR (400 MHz, DMSO-d6) δ 10.26 (s, 1H), 9.33 (s, 1H), 8.92 (d, J = 2.2 Hz, 1H), 8.58 (s, 1H), 8.47 (s, 1H), 8.25- 8.03 (m, 2H), 7.79 (dd, J = 8.8, 2.2 Hz, 1H), 7.60 (d, J = 1.4 Hz, 1H), 7.49 (d, J = 1.6 Hz, 1H), 5.25-4.82 (m, 2H), 3.93 (s, 3H), 3.38 (d, J = 5.9 Hz, 1H), 2.97 (d, J = 12.9 Hz, 1H), 2.81-2.63 (m, 2H), 2.46-2.28 (m, 1H), 1.75 (tt, J = 11.6, 6.0 Hz, 1H). |
| 348 | 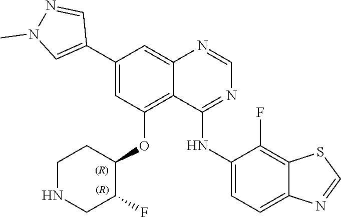 | 7-fluoro-N-(5-(((3R,4R)-3-fluoropiperidin-4-yl)oxy)-7-(1-methyl- 1H-pyrazol-4-yl)quinazolin-4-yl)benzo[d]thiazol-6-amine LC-MS: (ES, m/z): RT = 1.350 min, LCMS: m/z = 494 [M + 1], ¹H NMR (300 MHz, DMSO-d6) δ 10.20 (s, 1H), 9.43 (d, J = 1.6 Hz, 1H), 8.75 (dd, J = 8.9, 7.9 Hz, 1H), 8.56 (s, 1H), 8.49 (s, 1H), 8.19 (d, J = 0.8 Hz, 1H), 8.04 (d, J = 8.9 Hz, 1H), 7.62 (d, J = 1.4 Hz, 1H), 7.54 (s, 1H), 5.19 (d, J = 11.5 Hz, 1H), 4.99-4.68 (m, 1H), 3.93 (s, 3H), 3.40 (s, 1H), 2.95 (d, J = 13.0 Hz, 1H), 2.68 (t, J = 11.9 Hz, 2H), 2.35 (d, J = 14.0 Hz, 2H), 1.70 (d, J = 10.3 Hz, 1H). |
| 349 | 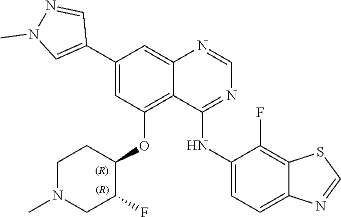 | 7-fluoro-N-(5-(((3R,4R)-3-fluoro-1-methylpiperidin-4-yl)oxy)-7-(1- methyl-1H-pyrazol-4-yl)quinazolin-4-yl)benzo[d]thiazol-6-amine LC-MS: (ES, m/z): RT = 1.500 min, LCMS: m/z = 508 [M + 1], ¹H NMR (300 MHz, DMSO-d6) δ 10.12 (s, 1H), 9.43 (d, J = 1.6 Hz, 1H), 8.70 (dd, J = 8.9, 7.9 Hz, 1H), 8.54 (s, 1H), 8.49 (s, 1H), 8.19 (d, J = 0.8 Hz, 1H), 8.04 (d, J = 9.0 Hz, 1H), 7.62 (d, J = 1.4 Hz, 1H), 7.53 (d, J = 1.6 Hz, 1H), 5.17-4.87 (m, 2H), 3.93 (s, 3H), 3.23 (d, J = 8.1 Hz, 1H), 2.79 (d, J = 11.3 Hz, 1H), 2.30 (s, 6H), 1.88 (d, J = 11.9 Hz, 1H). |
| 350 | 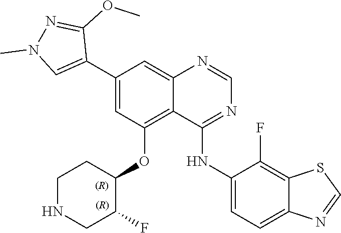 | 7-fluoro-N-(5-(((3R,4R)-3-fluoropiperidin-4-yl)oxy)-7-(3-methoxy- 1-methyl-1H-pyrazol-4-yl)quinazolin-4-yl)benzo[d]thiazol-6-amine LC-MS: (ES, m/z): RT = 1.451 min, LCMS: m/z = 524 [M + 1], ¹H NMR (300 MHz, DMSO-d6) δ 10.16 (s, 1H), 9.43 (d, J = 1.6 Hz, 1H), 8.81-8.69 (m, 1H), 8.53 (s, 1H), 8.37 (s, 1H), 8.04 (d, J = 9.0 Hz, 1H), 7.72 (d, J = 1.4 Hz, 1H), 7.52 (d, J = 1.5 Hz, 1H), 4.89 (d, J = 66.5 Hz, 2H), 3.99 (s, 3H), 3.79 (s, 3H), 3.37 (s, 1H), 2.97 (d, J = 13.2 Hz, 1H), 2.66 (t, J = 12.3 Hz, 2H), 2.33 (s, 1H), 1.69 (s, 1H). |
| 351 | 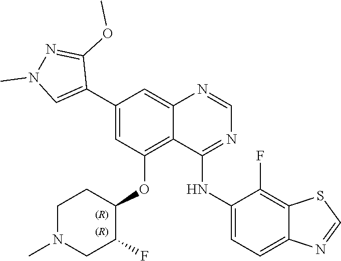 | 7-fluoro-N-(5-(((3R,4R)-3-fluoro-1-methylpiperidin-4-yl)oxy)-7-(3- methoxy-1-methyl-1H-pyrazol-4-yl)quinazolin-4-yl)benzo[d]thiazol- 6-amine LC-MS: (ES, m/z): RT = 1.628 min, LCMS: m/z = 538 [M + 1], ¹H NMR (300 MHz, DMSO-d6) δ 10.08 (s, 1H), 9.43 (d, J = 1.6 Hz, 1H), 8.75-8.63 (m, 1H), 8.52 (s, 1H), 8.37 (s, 1H), 8.03 (d, J = 8.9 Hz, 1H), 7.72 (d, J = 1.4 Hz, 1H), 7.51 (d, J = 1.5 Hz, 1H), 5.20- 4.85 (m, 2H), 3.99 (s, 3H), 3.79 (s, 3H), 3.23 (s, 1H), 2.80 (d, J = 11.4 Hz, 1H), 2.30 (s, 6H), 1.87 (d, J = 10.0 Hz, 1H). |
| 352 | 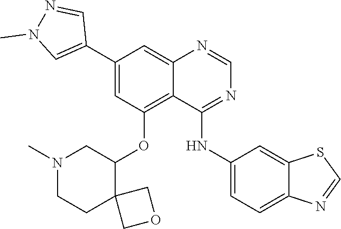 [SMILES: CN1CCC2(COC2)C(Oc2cc(-c3cnn(C)c3)cc3ncnc(Nc4ccc5ncsc5c4)c23)C1] | N-(7-(1-methyl-1H-pyrazol-4-yl)-5-((7-methyl-2-oxa-7- azaspiro[3.5]nonan-5-yl)oxy)quinazolin-4-yl)benzo[d]thiazol-6- amine LC-MS: (ES, m/z): RT = 0.822 min, m/z = 514 [M + 1], 1H NMR (400 MHz, DMSO-d6) δ 10.54 (s, 1H), 9.32 (s, 1H), 8.75-8.68 (m, 1H), 8.50 (d, J = 3.8 Hz, 2H), 8.20 (d, J = 0.8 Hz, 1H), 8.12 (d, J = 8.8 Hz, 1H), 7.76-7.68 (m, 2H), 7.60 (d, J = 1.4 Hz, 1H), 5.66 (s, 1H), 4.62 (t, J = 5.9 Hz, 2H), 4.45 (d, J = 6.1 Hz, 1H), 4.29 (d, J = 6.2 Hz, 1H), 3.93 (s, 3H), 2.88 (s, 1H), 2.73 (s, 1H), 2.25-2.15 (m, 1H), 2.13 (s, 6H). |
| 353 354 | 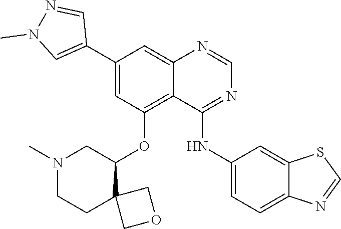 [SMILES: CN1CCC2(COC2)[C@H](Oc2cc(-c3cnn(C)c3)cc3ncnc(Nc4ccc5ncsc5c4)c23)C1] or 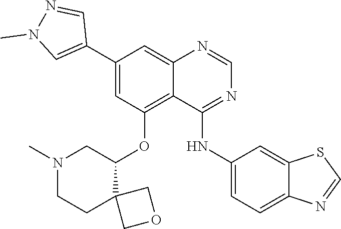 [SMILES: CN1CCC2(COC2)[C@@H](Oc2cc(-c3cnn(C)c3)cc3ncnc(Nc4ccc5ncsc5c4)c23)C1] | (S)-N-(7-(1-methyl-1H-pyrazol-4-yl)-5-((7-methyl-2-oxa-7- azaspiro[3.5]nonan-5-yl)oxy)quinazolin-4-yl)benzo[d]thiazol-6- amine OR (R)-N-(7-(1-methyl-1H-pyrazol-4-yl)-5-((7-methyl-2- oxa-7-azaspiro[3.5]nonan-5-yl)oxy)quinazolin-4-yl)benzo[d]thiazol- 6-amine First Eluting Isomer: LC-MS: (ES, m/z): RT = 0.994 min, LCMS: m/z = 514 [M + 1], Chiral-HPLC (ES): RT = 1.95,¹H NMR (300 MHz, DMSO-d₆) δ 10.54 (s, 1H), 9.32 (s, 1H), 8.70 (d, J = 1.8 Hz, 1H), 8.50 (d, J = 3.6 Hz, 2H), 8.20 (s, 1H), 8.11 (d, J = 9 Hz, 1H), 7.73- 7.69 (m, 2H), 7.59 (s, 1H), 5.76 (s, 1H), 4.63-4.60 (m, 2H), 4.44 (d, J = 6.3 Hz, 1H), 4.28 (d, J = 6 Hz, 1H), 3.93 (s, 3H), 2.13 (s, 6H), 2.00 (s, 2H). Second Eluting Isomer: LC-MS: (ES, m/z): RT = 0.989 min, LCMS: m/z = 514 [M + 1], Chiral-HPLC (ES): RT = 2.60, ¹H NMR (300 MHz, DMSO-d₆) δ 10.54 (s, 1H), 9.32 (s, 1H), 8.70 (d, J = 2.1 Hz, 1H), 8.50 (d, J = 3.3 Hz, 2H), 8.20 (s, 1H), 8.12 (d, J = 8.7 Hz, 1H), 7.73-7.69 (m, 2H), 7.59 (s, 1H), 5.66 (s, 1H), 4.64-4.60 (m, 2H), 4.44 (d, J = 6 Hz, 1H), 4.28 (d, J = 6 Hz, 1H), 3.93 (s, 3H), 2.73 (m, 1H), 2.13 (s, 6H), 2.00 (m, 1H). |
| 356 | 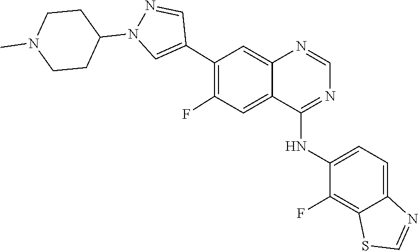 [SMILES: CN1CCC(n2cc(-c3cc4ncnc(Nc5ccc6ncsc6c5F)c4cc3F)cn2)CC1] | 7-fluoro-N-(6-fluoro-7-(1-(1-methylpiperidin-4-yl)-1H-pyrazol-4- yl)quinazolin-4-yl)benzo[d]thiazol-6-amine LC-MS: (ES, m/z): RT = 1.119 min, LCMS: m/z = 478 [M + 1], ¹H NMR (300 MHz, DMSO-d6) δ 9.98 (s, 1H), 9.47 (d, J = 1.5 Hz, 1H), 8.51-8.43 (m, 2H), 8.39 (d, J = 12.1 Hz, 1H), 8.19 (d, J = 7.5 Hz, 1H), 8.14 (d, J = 2.0 Hz, 1H), 8.01 (d, J = 8.7 Hz, 1H), 7.71 (t, J = 8.1 Hz, 1H), 4.20 (d, J = 8.2 Hz, 1H), 2.86 (d, J = 7.0 Hz, 2H), |
| 358 359 | 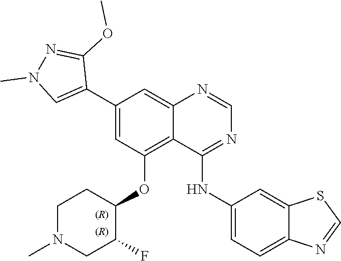 or 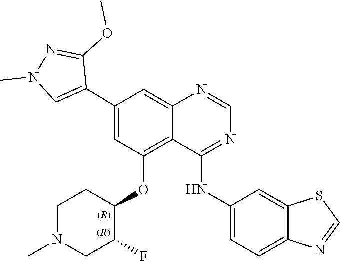 | N-(5-(((3R,4R)-3-fluoro-1-methylpiperidin-4-yl)oxy)-7-(3-methoxy- 1-methyl-1H-pyrazol-4-yl)quinazolin-4-yl)benzo[d]thiazol-6-amine OR N-(5-(((3S,4S)-3-fluoro-1-methylpiperidin-4-yl)oxy)-7-(3- methoxy-1-methyl-1H-pyrazol-4-yl)quinazolin-4-yl)benzo[d]thiazol- 6-amine First Eluting Isomer: LC-MS: (ES, m/z): RT = 0.863 min, LCMS: m/z = 520 [M + 1]. ¹H NMR (400 MHz, DMSO-d6) δ 10.14 (s, 1H), 9.31 (s, 1H), 8.89 (d, J = 2.2 Hz, 1H), 8.55 (s, 1H), 8.34 (s, 1H), 8.10 (d, J = 8.8 Hz, 1H), 7.82 (dd, J = 8.8, 2.2 Hz, 1H), 7.69 (d, J = 1.4 Hz, 1H), 7.47 (d, J = 1.5 Hz, 1H), 5.16 (dtd, J = 50.8, 8.8, 4.9 Hz, 1H), 4.90 (q, J = 13.1, 9.8 Hz, 1H), 3.98 (s, 3H), 3.78 (s, 3H), 3.21 (td, J = 9.7, 7.1, 4.2 Hz, 1H), 2.80 (d, J = 11.4 Hz, 1H), 2.45-2.20 (m, 5H), 1.99-1.81 (m, 1H). Second Eluting Isomer: LC-MS: (ES, m/z): RT = 1.523 min, LCMS: m/z = 520 [M + 1]. ¹H NMR (400 MHz, DMSO-d6) δ 10.14 (s, 1H), 9.31 (s, 1H), 8.89 (d, J = 2.2 Hz, 1H), 8.55 (s, 1H), 8.34 (s, 1H), 8.10 (d, J = 8.8 Hz, 1H), 7.82 (dd, J = 8.9, 2.2 Hz, 1H), 7.69 (d, J = 1.4 Hz, 1H), 7.47 (d, J = 1.5 Hz, 1H), 5.16 (dtd, J = 50.7, 8.9, 4.9 Hz, 1H), 4.96-4.82 (m, 1H), 3.98 (s, 3H), 3.78 (s, 3H), 3.25-3.13 (m, 1H), 2.80 (d, J = 11.4 Hz, 1H), 2.42-2.19 (m, 6H), 2.01-1.85 (m, 1H). |
| 360 361 | 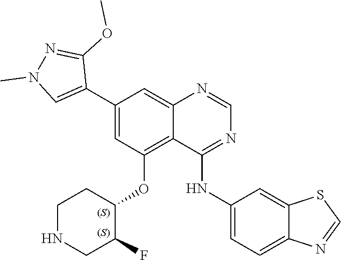 or 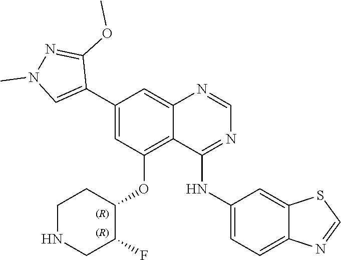 | N-(5-(((3S,4S)-3-fluoropiperidin-4-yl)oxy)-7-(3-methoxy-1-methyl- 1H-pyrazol-4-yl)quinazolin-4-yl)benzo[d]thiazol-6-amine OR N- (5-(((3R,4R)-3-fluoropiperidin-4-yl)oxy)-7-(3-methoxy-1-methyl- 1H-pyrazol-4-yl)quinazolin-4-yl)benzo[d]thiazol-6-amine First Eluting Isomer: LC-MS: (ES, m/z): RT = 1.390 min, LCMS: m/z = 506 [M + 1]. ¹H NMR (400 MHz, DMSO-d6) δ 10.21 (s, 1H), 9.30 (s, 1H), 8.90 (d, J = 2.2 Hz, 1H), 8.53 (s, 1H), 8.33 (s, 1H), 8.09 (d, J = 8.7 Hz, 1H), 7.76 (dd, J = 8.8, 2.2 Hz, 1H), 7.67 (d, J = 1.4 Hz, 1H), 7.47 (d, J = 1.6 Hz, 1H), 5.10-4.83 (m, 2H), 3.96 (s, 3H), 3.77 (s, 3H), 2.95 (d, J = 12.8 Hz, 1H), 2.66 (d, J = 12.2 Hz, 2H), 2.35 (d, J = 32.2 Hz, 2H), 1.73 (d, J = 11.9 Hz, 1H). Second Eluting Isomer: LC-MS: (ES, m/z): RT = 1.390 min, LCMS: m/z = 506 [M + 1]. ¹H NMR (400 MHz, DMSO-d6) δ 10.21 (s, 1H), 9.30 (s, 1H), 8.90 (d, J = 2.2 Hz, 1H), 8.53 (s, 1H), 8.33 (s, 1H), 8.09 (d, J = 8.7 Hz, 1H), 7.76 (dd, J = 8.8, 2.2 Hz, 1H), 7.67 (d, J = 1.4 Hz, 1H), 7.47 (d, J = 1.6 Hz, 1H), 5.10-4.83 (m, 2H), 3.96 (s, 3H), 3.77 (s, 3H), 2.95 (d, J = 12.8 Hz, 1H), 2.66 (d, J = 12.2 Hz, 2H), 2.35 (d, J = 32.2 Hz, 2H), 1.73 (d, J = 11.9 Hz, 1H). |
| 362 | 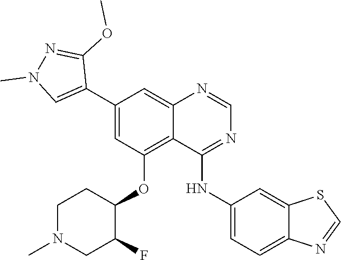 [SMILES: COc1nn(C)cc1-c1cc(O[C@@H]2CCN(C)C[C@@H]2F)c2c(Nc3ccc4ncsc4c3)ncnc2c1] | N-(5-(((3S,4R)-3-fluoro-1-methylpiperidin-4-yl)oxy)-7-(3-methoxy- 1-methyl-1H-pyrazol-4-yl)quinazolin-4-yl)benzo[d]thiazol-6-amine LC-MS: (ES, m/z): RT = 1.323 min, LCMS: m/z = 520 [M + 1], ¹H NMR (300 MHz, DMSO-d6) δ 10.34 (s, 1H), 9.32 (s, 1H), 8.97 (d, J = 2.1 Hz, 1H), 8.57 (s, 1H), 8.34 (s, 1H), 8.11 (d, J = 8.8 Hz, 1H), 7.72 (dd, J = 8.3, 1.8 Hz, 2H), 7.46 (s, 1H), 5.35-4.98 (m, 2H), 3.99 (s, 3H), 3.79 (s, 3H), 3.19 (s, 1H), 2.86 (d, J = 11.6 Hz, 1H), 2.26 (s, 5H), 2.17-2.04 (m, 2H). |
| 363 | 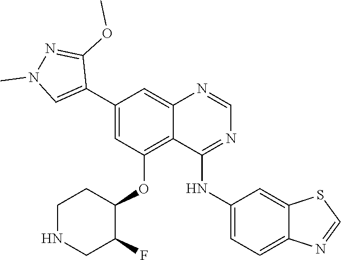 [SMILES: COc1nn(C)cc1-c1cc(O[C@@H]2CCNC[C@@H]2F)c2c(Nc3ccc4ncsc4c3)ncnc2c1] | N-(5-(((3S,4R)-3-fluoropiperidin-4-yl)oxy)-7-(3-methoxy-1-methyl- 1H-pyrazol-4-yl)quinazolin-4-yl)benzo[d]thiazol-6-amine LC-MS: (ES, m/z): RT = 1.979 min, LCMS: m/z = 506 [M + 1], ¹H NMR (300 MHz, DMSO-d6) δ 10.41 (s, 1H), 9.32 (s, 1H), 8.98 (d, J = 2.1 Hz, 1H), 8.57 (s, 1H), 8.34 (s, 1H), 8.10 (d, J = 8.8 Hz, 1H), 7.80-7.67 (m, 2H), 7.47 (s, 1H), 5.15 (d, J = 50.5 Hz, 2H), 3.99 (s, 3H), 3.79 (s, 3H), 3.10-2.63 (m, 4H), 2.16-1.76 (m, 2H). |
| 364 | 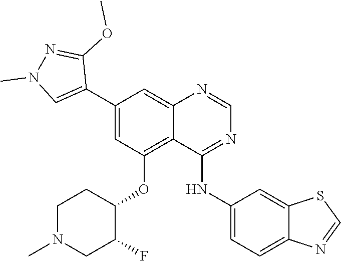 [SMILES: COc1nn(C)cc1-c1cc(O[C@H]2CCN(C)C[C@H]2F)c2c(Nc3ccc4ncsc4c3)ncnc2c1] | N-(5-(((3R,4S)-3-fluoro-1-methylpiperidin-4-yl)oxy)-7-(3-methoxy- 1-methyl-1H-pyrazol-4-yl)quinazolin-4-yl)benzo[d]thiazol-6-amine LC-MS: (ES, m/z): RT = 1.319 min, LCMS: m/z = 520 [M + 1], ¹H NMR (300 MHz, Methanol-d4) δ 9.19 (s, 1H), 8.96 (s, 1H), 8.55 (s, 1H), 8.11-8.00 (m, 2H), 7.88-7.72 (m, 2H), 7.52 (s, 1H), 5.29 (d, J = 49.1 Hz, 2H), 4.06 (s, 3H), 3.84 (s, 3H), 3.58-3.39 (m, 2H),2.54 (s, 1H), 2.47 (m, 5H), 2.35 (s, 1H). |
| 365 | 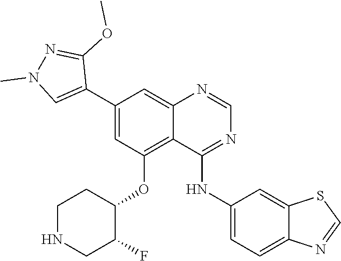 [SMILES: COc1nn(C)cc1-c1cc(O[C@H]2CCNC[C@H]2F)c2c(Nc3ccc4ncsc4c3)ncnc2c1] | N-(5-(((3R,4S)-3-fluoropiperidin-4-yl)oxy)-7-(3-methoxy-1-methyl- 1H-pyrazol-4-yl)quinazolin-4-yl)benzo[d]thiazol-6-amine LC-MS: (ES, m/z): RT = 2.007 min, LCMS: m/z = 506 [M + 1], ¹H NMR (300 MHz, DMSO-d6) δ 10.41 (s, 1H), 9.32 (s, 1H), 8.98 (d, J = 2.2 Hz, 1H), 8.57 (s, 1H), 8.34 (s, 1H), 8.11 (d, J = 8.9 Hz, 1H), 7.80-7.67 (m, 2H), 7.48 (s, 1H), 5.15 (d, J = 51.6 Hz, 2H), 3.99 (s, 3H), 3.79 (s, 3H), 3.11-2.65 (m, 4H), 1.98 (d, J = 70.2 Hz, 2H).. |
| 366 | 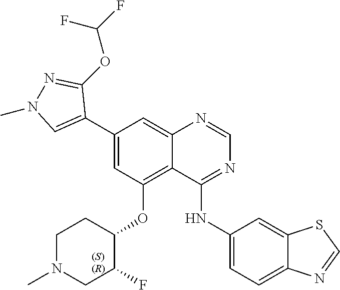 | N-(7-(3-(difluoromethoxy)-1-methyl-1H-pyrazol-4-yl)-5-(((3R,4S)- 3-fluoropiperidin-4-yl)oxy)quinazolin-4-yl)benzo[d]thiazol-6-amine LC-MS: (ES, m/z): RT = 1.504 min, LCMS: m/z = 542 [M + 1], ¹H NMR (300 MHz, Methanol-d4) δ 9.20 (s, 1H), 9.01 (d, J = 2.1 Hz, 1H), 8.60 (s, 1H), 8.16 (d, J = 2.8 Hz, 1H), 8.04 (d, J = 8.9 Hz, 1H), 7.76 (dd, J = 8.8, 2.2 Hz, 1H), 7.69 (d, J = 1.3 Hz, 1H), 7.54-7.00 (m, 2H), 5.59 (d, J = 48.0 Hz, 1H), 5.28 (d, J = 26.0 Hz, 1H), 3.88 (s, 4H), 3.62 (d, J = 14.8 Hz, 2H), 3.49 (d, J = 14.4 Hz, 1H), 2.63 (d, J = 13.6 Hz, 1H), 2.35 (d, J = 12.9 Hz, 1H). |
| 367 | 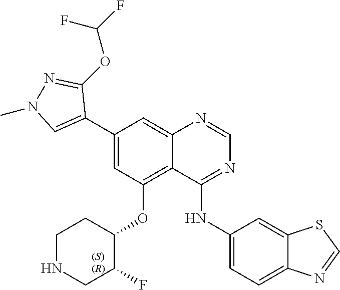 | N-(7-(3-(difluoromethoxy)-1-methyl-1H-pyrazol-4-yl)-5-(((3R,4S)- 3-fluoro-1-methylpiperidin-4-yl)oxy)quinazolin-4- yl)benzo[d]thiazol-6-amine LC-MS: (ES, m/z): RT = 1.610 min, LCMS: m/z = 556 [M + 1], ¹H NMR (300 MHz, DMSO-d6) δ 10.36 (s, 1H), 9.33 (s, 1H), 8.99 (s, 1H), 8.61 (s, 1H), 8.49 (s, 1H), 8.12 (d, J = 8.8 Hz, 1H), 7.73 (d, J = 10.1 Hz, 1H), 7.63 (s, 1H), 7.34 (d, J = 72.2 Hz, 2H), 5.34 (s, 1H), 5.17 (s, 1H), 3.86 (s, 3H), 3.20 (s, 1H), 2.79 (d, J = 35.6 Hz, 1H), 2.26 (s, 7H), 1.24 (s, 1H). |
| 368 369 | 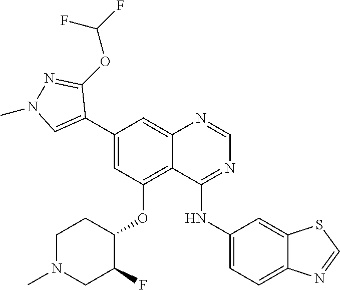 [SMILES: CN1CC[C@H](Oc2cc(-c3cn(C)nc3OC(F)F)cc3ncnc(Nc4ccc5ncsc5c4)c23)[C@@H](F)C1] or 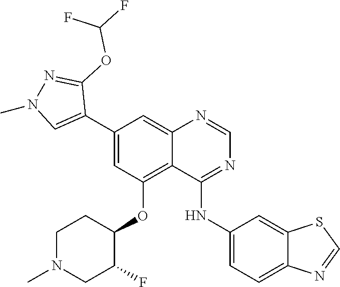 [SMILES: CN1CC[C@@H](Oc2cc(-c3cn(C)nc3OC(F)F)cc3ncnc(Nc4ccc5ncsc5c4)c23)[C@H](F)C1] | N-(7-(3-(difluoromethoxy)-1-methyl-1H-pyrazol-4-yl)-5-(((3S,4S)- 3-fluoro-1-methylpiperidin-4-yl)oxy)quinazolin-4- yl)benzo[d]thiazol-6-amine OR N-(7-(3-(difluoromethoxy)-1- methyl-1H-pyrazol-4-yl)-5-(((3R,4R)-3-fluoro-1-methylpiperidin-4- yl)oxy)quinazolin-4-yl)benzo[d]thiazol-6-amine First Eluting Isomer: LC-MS: (ES, m/z): RT = 1.656 min, LCMS: m/z = 556 [M + 1]. ¹H NMR (400 MHz, DMSO-d6) δ 10.16 (s, 1H), 9.32 (s, 1H), 8.89 (d, J = 2.2 Hz, 1H), 8.58 (s, 1H), 8.49 (s, 1H), 8.11 (d, J = 8.8 Hz, 1H), 7.84 (dd, J = 8.8, 2.2 Hz, 1H), 7.70-7.20 (m, 3H), 5.30-5.05 (m, 1H), 4.88 (s, 1H), 3.85 (s, 3H), 3.25 (s, 1H), 2.81 (d, J = 11.2 Hz, 1H), 2.42 (s, 1H), 2.30 (s, 4H), 2.22 (d, J = 11.5 Hz, 1H), 1.96 (d, J = 12.5 Hz, 1H). Second Eluting Isomer: LC-MS: (ES, m/z): RT = 1.656 min, LCMS: m/z = 556 [M + 1]. ¹H NMR (400 MHz, DMSO-d6) δ 10.16 (s, 1H), 9.32 (s, 1H), 8.89 (d, J = 2.2 Hz, 1H), 8.58 (s, 1H), 8.49 (s, 1H), 8.11 (d, J = 8.8 Hz, 1H), 7.84 (dd, J = 8.8, 2.2 Hz, 1H), 7.68-7.19 (m, 3H), 5.19 (dd, J = 50.7, 5.0 Hz, 1H), 3.85 (s, 3H), 3.24 (t, J = 5.4 Hz, 1H), 2.81 (d, J = 11.7 Hz, 1H), 2.39 (s, 1H), 2.30 (s, 5H), 1.95 (d, J = 11.6 Hz, 1H). |
| 370 | 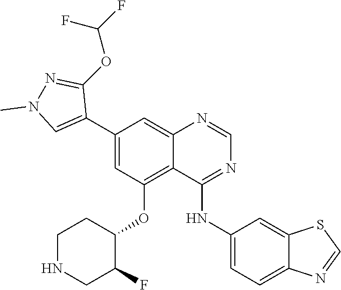 [SMILES: Cn1cc(-c2cc(O[C@H]3CCNC[C@@H]3F)c3c(Nc4ccc5ncsc5c4)ncnc3c2)c(OC(F)F)n1] | N-(7-(3-(difluoromethoxy)-1-methyl-1H-pyrazol-4-yl)-5-(((3S,4S)- 3-fluoropiperidin-4-yl)oxy)quinazolin-4-yl)benzo[d]thiazol-6-amine LC-MS: (ES, m/z): RT = 1.490 min, LCMS: m/z = 542 [M + 1]. ¹H NMR (400 MHz, DMSO-d6) δ 10.23 (s, 1H), 9.33 (s, 1H), 8.92 (d, J = 2.2 Hz, 1H), 8.59 (s, 1H), 8.48 (s, 1H), 8.12 (d, J = 8.8 Hz, 1H), 7.79 (dd, J = 8.8, 2.2 Hz, 1H), 7.66-7.21 (m, 3H), 5.12-4.91 (m, 2H), 3.85 (s, 3H), 3.38 (dt, J = 10.7, 4.8 Hz, 1H), 2.99 (d, J = 12.4 Hz, 1H), 2.75-2.59 (m, 2H), 2.41 (d, J = 12.5 Hz, 1H), 1.86-1.73 (m, 1H). |
| 371 | 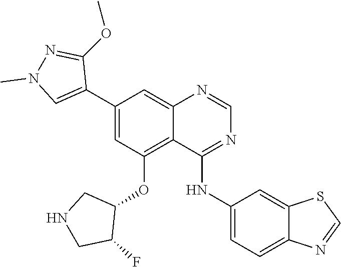 [SMILES: COc1nn(C)cc1-c1cc(O[C@H]2CNC[C@H]2F)c2c(Nc3ccc4ncsc4c3)ncnc2c1] cis racemic | N-(5-(((3S,4R)-4-fluoropyrrolidin-3-yl)oxy)-7-(3-methoxy-1- methyl-1H-pyrazol-4-yl)quinazolin-4-yl)benzo[d]thiazol-6-amine and N-(5-(((3R,4S)-4-fluoropyrrolidin-3-yl)oxy)-7-(3-methoxy-1- methyl-1H-pyrazol-4-yl)quinazolin-4-yl)benzo[d]thiazol-6-amine LC-MS: (ES, m/z): RT = 1.799 min, LCMS: m/z = 492 [M + 1], ¹H NMR (300 MHz, DMSO-d6) δ 9.28 (s, 1H), 8.93 (t, J = 2.5 Hz, 1H), 8.55 (s, 1H), 8.41-8.20 (m, 3H), 8.07 (d, J = 8.8 Hz, 1H), 7.72 (dd, J = 8.8, 2.2 Hz, 1H), 7.66 (s, 1H), 7.39 (d, J = 18.0 Hz, 1H), 5.51 (d, J = 54.5 Hz, 1H), 5.24 (s, 1H), 3.97 (s, 3H), 3.76 (d, J = 2.3 Hz, 3H), 3.23 (s, 2H), 2.98 (s, 1H). |
| 373 374 | 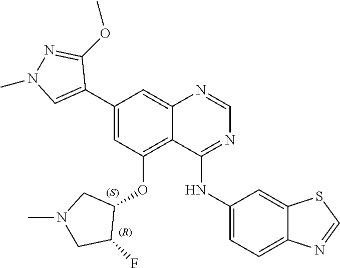 or 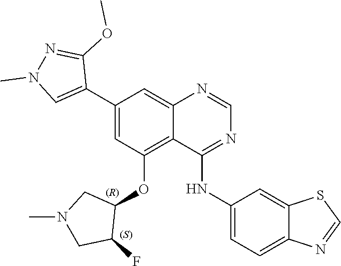 | N-(5-(((3S,4R)-4-fluoro-1-methylpyrrolidin-3-yl)oxy)-7-(3- methoxy-1-methyl-1H-pyrazol-4-yl)quinazolin-4-yl)benzo[d]thiazol- 6-amine OR N-(5-(((3R,4S)-4-fluoro-1-methylpyrrolidin-3-yl)oxy)- 7-(3-methoxy-1-methyl-1H-pyrazol-4-yl)quinazolin-4- yl)benzo[d]thiazol-6-amine First Eluting peak: LC-MS: (ES, m/z): RT = 1.503 min, LCMS: m/z = 506 [M + 1], HPLC: RT = 4.093 min, ¹H NMR (400 MHz, DMSO- d6) δ 9.29 (s, 1H), 8.94 (d, J = 5.4 Hz, 1H), 8.55 (s, 1H), 8.32 (d, J = 20.8 Hz, 1H), 8.08 (d, J = 8.8 Hz, 1H), 7.83-7.61 (m, 2H), 7.35 (s, 1H), 5.69-5.33 (m, 2H), 3.96 (s, 3H), 3.76 (s, 3H), 3.00 (s, 5H), 2.37 (s, 3H). Second eluting peak: LC-MS: (ES, m/z): RT = 1.496 min, LCMS: m/z = 506 [M + 1], HPLC: RT = 4.847 min, ¹H NMR (400 MHz, DMSO-d6) δ 9.29 (s, 1H), 8.93 (dd, J = 4.2, 2.1 Hz, 1H), 8.55 (s, 1H), 8.34 (s, 1H), 8.14-8.03 (m, 1H), 7.74 (dd, J = 8.8, 2.2 Hz, 1H), 7.67 (d, J = 1.4 Hz, 1H), 7.35 (d, J = 1.6 Hz, 1H), 5.68-5.34 (m, 2H), 3.96 (s, 3H), 3.76 (s, 3H), 3.19-2.96 (m, 4H), 2.39 (s, 3H). |
| 375 | 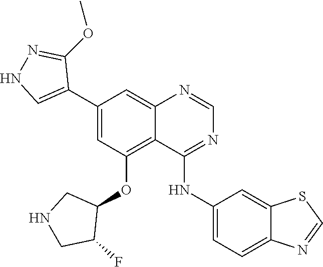 trans racemic | N-(5-(((3R,4R)-4-fluoropyrrolidin-3-yl)oxy)-7-(3-methoxy-1H- pyrazol-4-yl)quinazolin-4-yl)benzo[d]thiazol-6-amine and N-(5- (((3S,4S)-4-fluoropyrrolidin-3-yl)oxy)-7-(3-methoxy-1H-pyrazol-4- yl)quinazolin-4-yl)benzo[d]thiazol-6-amine LC-MS: (ES, m/z): RT = 0.990 min, LCMS: m/z = 492 [M + 1], ¹H NMR (300 MHz, DMSO-d6) δ 9.29 (s, 1H), 8.89 (dd, J = 3.9, 2.1 Hz, 1H), 8.53 (d, J = 1.5 Hz, 1H), 8.31 (s, 1H), 8.07 (d, J = 8.8 Hz, 1H), 7.82 (dd, J = 8.9, 2.2 Hz, 1H), 7.66 (d, J = 1.4 Hz, 1H), 7.47 (d, J = 1.6 Hz, 1H), 5.57-5.26 (m, 1H), 3.95 (s, 3H), 3.76 (s, 3H), 3.57- 3.42 (m, 2H), 3.21 (d, J = 4.7 Hz, 1H), 3.19-3.12 (m, 2H), 3.08 (d, J = 14.0 Hz, 2H). |
| 376 | 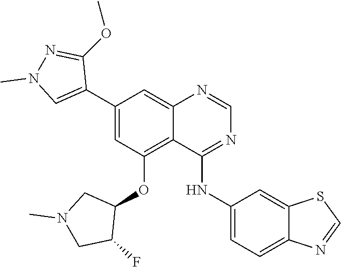 [SMILES: COc1nn(C)cc1-c1cc(O[C@@H]2CN(C)C[C@H]2F)c2c(Nc3ccc4ncsc4c3)ncnc2c1] trans racemic | N-(5-(((3S,4S)-4-fluoro-1-methylpyrrolidin-3-yl)oxy)-7-(3-methoxy- 1-methyl-1H-pyrazol-4-yl)quinazolin-4-yl)benzo[d]thiazol-6-amine and N-(5-(((3R,4R)-4-fluoro-1-methylpyrrolidin-3-yl)oxy)-7-(3- methoxy-1-methyl-1H-pyrazol-4-yl)quinazolin-4-yl)benzo[d]thiazol- 6-amine LC-MS: (ES, m/z): RT = 1.009 min, LCMS: m/z = 506 [M + 1], ¹H NMR (300 MHz, DMSO-d6) δ 10.13 (s, 1H), 9.29 (s, 1H), 8.88 (d, J = 2.1 Hz, 1H), 8.54 (s, 1H), 8.30 (s, 1H), 8.08 (d, J = 8.8 Hz, 1H), 7.79 (dd, J = 8.8, 2.2 Hz, 1H), 7.67 (d, J = 1.4 Hz, 1H), 7.49 (s, 1H), 5.49-5.26 (m, 2H), 3.95 (s, 3H), 3.76 (s, 3H), 3.18-2.99 (m, 2H), 2.62 (dd, |
| 377 378 | 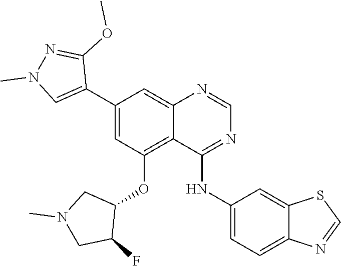 [SMILES: COc1nn(C)cc1-c1cc(O[C@H]2CN(C)C[C@@H]2F)c2c(Nc3ccc4ncsc4c3)ncnc2c1] or 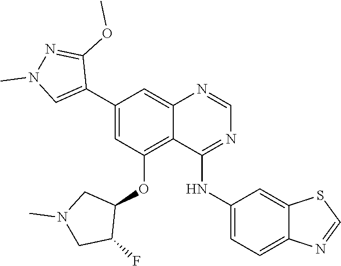 [SMILES: COc1nn(C)cc1-c1cc(O[C@@H]2CN(C)C[C@H]2F)c2c(Nc3ccc4ncsc4c3)ncnc2c1] | N-(5-(((3S,4S)-4-fluoro-1-methylpyrrolidin-3-yl)oxy)-7-(3-methoxy- 1-methyl-1H-pyrazol-4-yl)quinazolin-4-yl)benzo[d]thiazol-6-amine OR N-(5-(((3R,4R)-4-fluoro-1-methylpyrrolidin-3-yl)oxy)-7-(3- methoxy-1-methyl-1H-pyrazol-4-yl)quinazolin-4-yl)benzo[d]thiazol- 6-amine First Eluting peak: LC-MS: (ES, m/z): RT = 1.623 min, LCMS: m/z = 506 [M + 1], ¹H NMR (400 MHz, DMSO-d6) δ 10.13 (s, 1H), 9.29 (s, 1H), 8.89 (d, J = 2.1 Hz, 1H), 8.54 (s, 1H), 8.31 (s, 1H), 8.08 (d, J = 8.9 Hz, 1H), 7.79 (dd, J = 8.8, 2.2 Hz, 1H), 7.68 (d, J = 1.4 Hz, 1H), 7.49 (d, J = 1.5 Hz, 1H), 5.42 (d, J = 4.0 Hz, 1H), 5.37 (s, 1H), 3.95 (s, 3H), 3.76 (s, 3H), 3.10 (dd, J = 10.8, 4.8 Hz, 1H), 3.07-3.00 (m, 1H), 2.69-2.55 (m, 2H), 2.41 (s, 3H). Second eluting peak: LC-MS: (ES, m/z): RT = 1.606 min, LCMS: m/z = 506 [M + 1], ¹H NMR (400 MHz, DMSO-d6) δ 10.13 (s, 1H), 9.29 (s, 1H), 8.89 (d, J = 2.1 Hz, 1H), 8.54 (s, 1H), 8.31 (s, 1H), 8.08 (d, J = 8.8 Hz, 1H), 7.79 (dd, J = 8.9, 2.2 Hz, 1H), 7.67 (d, J = 1.4 Hz, 1H), 7.49 (d, J = 1.6 Hz, 1H), 5.45-5.35 (m, 1H), 5.32-5.26 (m, 1H), 3.95 (s, 3H), 3.76 (s, 3H), 3.32-3.22 (m, 1H), 3.10 (dd, J = 10.8, 4.8 Hz, 1H), 2.69-2.55 (m, 2H), 2.41 (s, 3H). |
| 379 | 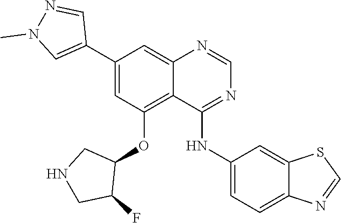 [SMILES: Cn1cc(-c2cc(O[C@@H]3CNC[C@@H]3F)c3c(Nc4ccc5ncsc5c4)ncnc3c2)cn1] racemic | N-(5-(((3R,4S)-4-fluoropyrrolidin-3-yl)oxy)-7-(1-methyl-1H- pyrazol-4-yl)quinazolin-4-yl)benzo[d]thiazol-6-amine LC-MS: (ES, m/z): RT = 1.027 min, LCMS: m/z = 462 [M + 1], ¹H NMR (300 MHz, DMSO-d6) δ 9.29 (s, 1H), 8.94 (t, J = 2.6 Hz, 1H), 8.50 (d, J = 36.4 Hz, 2H), 8.21-7.99 (m, 2H), 7.85-7.28 (m, 3H), 5.70-5.18 (m, 2H), 4.05-3.49 (m, 4H), 3.26-2.89 (m, 3H). |
| 380 381 | 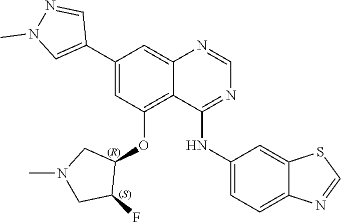 or 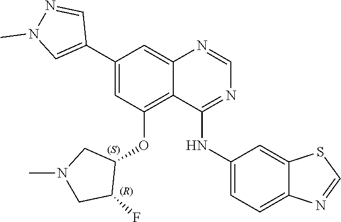 | N-(5-(((3R,4S)-4-fluoro-1-methylpyrrolidin-3-yl)oxy)-7-(1-methyl- 1H-pyrazol-4-yl)quinazolin-4-yl)benzo[d]thiazol-6-amine OR N- (5-(((3S,4R)-4-fluoro-1-methylpyrrolidin-3-yl)oxy)-7-(1-methyl-1H- pyrazol-4-yl)quinazolin-4-yl)benzo[d]thiazol-6-amine First Eluting Peak: LC-MS: (ES, m/z): RT = 0.985 min, LCMS: m/z = 476 [M + 1], HPLC: RT = 3.079 min, ¹H NMR (300 MHz, Methanol-d4) δ 9.15 (s, 1H), 8.90 (d, J = 2.1 Hz, 1H), 8.52 (s, 1H), 8.18 (s, 1H), 8.00 (d, J = 8.8 Hz, 2H), 7.80 (dd, J = 8.9, 2.2 Hz, 1H), 7.49 (d, J = 1.3 Hz, 1H), 7.20 (s, 1H), 5.69-5.35 (m, 2H), 3.95 (s, 3H), 3.25-3.03 (m, 3H), 2.88 (ddd, J = 32.8, 12.1, 4.4 Hz, 1H), 2.48 (s, 3H). Second Eluting Peak: LC-MS: (ES, m/z): RT = 0.986 min, LCMS: m/z = 476 [M + 1], HPLC: RT = 3.966 min, ¹H NMR (300 MHz, Methanol-d4) δ 8.18 (s, 1H), 8.00 (d, J = 8.7 Hz, 2H), 7.80 (dd, J = 8.8, 2.1 Hz, 1H), 7.49 (s, 1H), 7.19 (s, 1H), 5.55 (d, J = 59.1 Hz, 2H), 4.56 (s, 2H), 3.95 (s, 3H), 3.23-3.04 (m, 3H), 2.97-2.81 (m, 1H), 2.48 (s, 3H). |
| 382 | 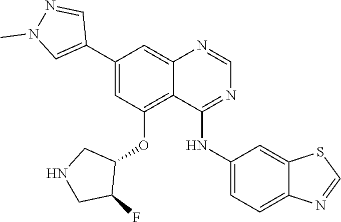 [SMILES: Cn1cc(-c2cc(O[C@H]3CNC[C@@H]3F)c3c(Nc4ccc5ncsc5c4)ncnc3c2)cn1] | N-(5-(((3S,4S)-4-fluoropyrrolidin-3-yl)oxy)-7-(1-methyl-1H- pyrazol-4-yl)quinazolin-4-yl)benzo[d]thiazol-6-amine LC-MS: (ES, m/z): RT-0.967 min, LCMS: m/z = 462 [M + 1], ¹H NMR (300 MHz, DMSO-d6) δ 9.99 (s, 1H), 9.29 (s, 1H), 8.89 (d, J = 2.1 Hz, 1H), 8.55 (s, 1H), 8.43 (s, 1H), 8.16-8.03 (m, 2H), 7.84 (dd, J = 8.8, 2.2 Hz, 1H), 7.60 (d, J = 1.4 Hz, 1H), 7.43 (d, J = 1.6 Hz, 1H), 5.58-5.35 (m, 2H), 3.90 (s, 3H), 3.47 (d, J = 13.1 Hz, 1H), 3.24-3.01 (m, 3H). |
| 383 | 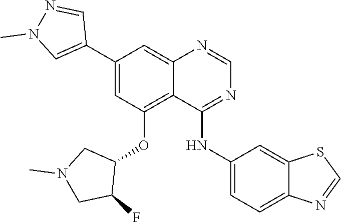 [SMILES: CN1C[C@H](Oc2cc(-c3cnn(C)c3)cc3ncnc(Nc4ccc5ncsc5c4)c23)[C@@H](F)C1] | N-(5-(((3S,4S)-4-fluoro-1-methylpyrrolidin-3-yl)oxy)-7-(1-methyl- 1H-pyrazol-4-yl)quinazolin-4-yl)benzo[d]thiazol-6-amine LC-MS: (ES, m/z): RT = 0.992 min, LCMS: m/z = 476 [M + 1], ¹H NMR (300 MHz, DMSO-d6) δ 9.28 (s, 1H), 8.88 (dd, J = 3.5, 2.1 Hz, 1H), 8.56 (d, J = 1.3 Hz, 1H), 8.41 (s, 1H), 8.08 (d, J = 8.4 Hz, 2H), 7.80 (dt, J = 8.9, 1.8 Hz, 1H), 7.60 (d, J = 1.4 Hz, 1H), 7.46 (d, J = 1.5 Hz, 1H), 5.64-5.24 (m, 2H), 3.89 (s, 3H), 3.06 (t, J = 3.0 Hz, 2H), 2.67 (d, J = 3.2 Hz, 2H), 2.41 (s, 3H). |
| 384 385 | 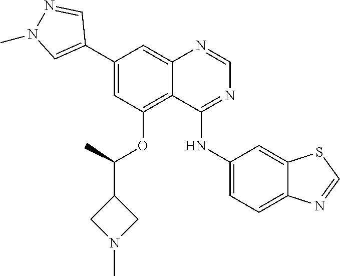 [SMILES: C[C@@H](Oc1cc(-c2cnn(C)c2)cc2ncnc(Nc3ccc4ncsc4c3)c12)C1CN(C)C1] or 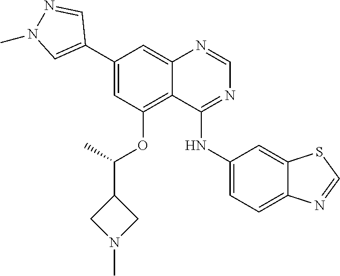 [SMILES: C[C@H](Oc1cc(-c2cnn(C)c2)cc2ncnc(Nc3ccc4ncsc4c3)c12)C1CN(C)C1] | (R)-N-(7-(1-methyl-1H-pyrazol-4-yl)-5-(1-(1-methylazetidin-3- yl)ethoxy)quinazolin-4-yl)benzo[d]thiazol-6-amine OR (S)-N-(7- (1-methyl-1H-pyrazol-4-yl)-5-(1-(1-methylazetidin-3- yl)ethoxy)quinazolin-4-yl)benzo[d]thiazol-6-amine First Eluting Peak: LC-MS: (ES, m/z): RT = 1.323 min, LCMS: m/z = 472 [M + 1]; ¹H NMR (300 MHz, DMSO-d6) δ 10.67 (s, 1H), 9.34 (s, 1H), 8.62 (d, J = 2.1 Hz, 1H), 8.47 (d, J = 10.4 Hz, 2H), 8.16 (d, J = 0.8 Hz, 1H), 8.10 (d, J = 8.8 Hz, 1H), 7.67 (dd, J = 8.8, 2.1 Hz, 1H), 7.54 (d, J = 1.4 Hz, 1H), 7.38 (d, J = 1.5 Hz, 1H), 5.21 (t, J = 6.0 Hz, 1H), 3.92 (s, 3H), 3.38 (s, 1H), 3.24 (t, J = 6.9 Hz, 3H), 2.85 (s, 1H), 2.01 (s, 3H), 1.40 (d, J = 5.9 Hz, 3H). Second Eluting Peak: LC-MS: (ES, m/z): RT = 1.340 min, LCMS: m/z = 472 [M + 1]; ¹H NMR (300 MHz, DMSO-d6) δ 10.67 (s, 1H), 9.34 (s, 1H), 8.62 (d, J = 2.0 Hz, 1H), 8.47 (d, J = 10.4 Hz, 2H), 8.16 (d, J = 0.8 Hz, 1H), 8.10 (d, J = 8.7 Hz, 1H), 7.67 (dd, J = 8.8, 2.2 Hz, 1H), 7.54 (d, J = 1.4 Hz, 1H), 7.38 (s, 1H), 5.21 (t, J = 6.0 Hz, 1H), 3.92 (s, 3H), 3.39 (d, J = 6.5 Hz, 1H), 3.24 (t, J = 6.8 Hz, 3H), 2.85 (s, 1H), 2.01 (s, 3H), 1.40 (d, J = 5.9 Hz, 3H). |
| 386 | 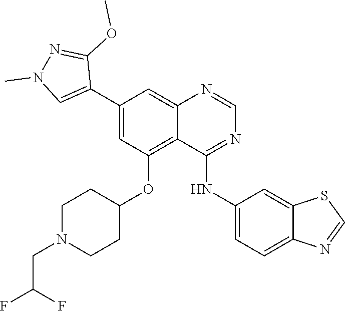 [SMILES: COc1nn(C)cc1-c1cc(OC2CCN(CC(F)F)CC2)c2c(Nc3ccc4ncsc4c3)ncnc2c1] | N-(5-((1-(2,2-difluoroethyl)piperidin-4-yl)oxy)-7-(3-methoxy-1- methyl-1H-pyrazol-4-yl)quinazolin-4-yl)benzo[d]thiazol-6-amine LC-MS: (ES, m/z): RT = 1.687 min, LCMS: m/z = 552 [M + 1], ¹H NMR (300 MHz, DMSO-d6) δ 10.28 (s, 1H), 9.33 (s, 1H), 8.90 (d, J = 2.1 Hz, 1H), 8.56 (s, 1H), 8.37 (s, 1H), 8.13 (d, J = 8.8 Hz, 1H), 7.75 (dd, J = 8.8, 2.2 Hz, 1H), 7.66 (d, J = 1.4 Hz, 1H), 7.43 (d, J = 1.4 Hz, 1H), 6.42-5.89 (m, 1H), 4.88 (s, 1H), 3.98 (s, 3H), 3.79 (s, 3H), 2.96 (d, J = 11.5 Hz, 2H), 2.81 (t, J = 17.0 Hz, 2H), 2.60 (d, J = 10.5 Hz, 2H), 2.26 (d, J = 12.7 Hz, 2H), 1.98 (d, J = 10.7 Hz, 2H). |
| 387 388 | 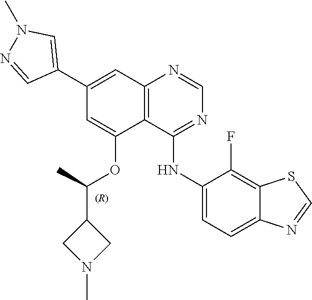 or 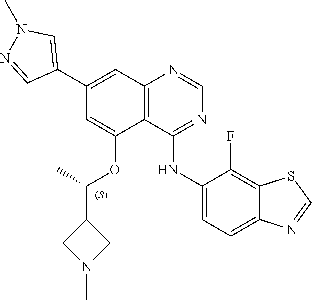 | (R)-7-fluoro-N-(7-(1-methyl-1H-pyrazol-4-yl)-5-(1-(1- methylazetidin-3-yl)ethoxy)quinazolin-4-yl)benzo[d]thiazol-6-amine OR (S)-7-fluoro-N-(7-(1-methyl-1H-pyrazol-4-yl)-5-(1-(1- methylazetidin-3-yl)ethoxy)quinazolin-4-yl)benzo[d]thiazol-6-amine First Eluting Peak: LC-MS: (ES, m/z): RT = 1.334 min, LCMS: m/z = 490 [M + 1]. ¹H NMR (300 MHz, DMSO-d6) δ 10.85 (s, 1H), 9.43 (d, J = 1.5 Hz, 1H), 8.46 (s, 1H), 8.35 (s, 1H), 8.14 (s, 1H), 8.07- 7.94 (m, 2H), 7.52 (d, J = 1.4 Hz, 1H), 7.33 (d, J = 1.3 Hz, 1H), 5.22- 5.07 (m, 1H), 3.90 (s, 3H), 3.50-3.37 (m, 1H), 3.25 (s, 1H), 3.17 (d, J = 5.7 Hz, 2H), 2.72 (s, 1H), 1.83 (s, 3H), 1.36 (d, J = 5.8 Hz, 3H). Second Eluting Peak: LC-MS: (ES, m/z): RT = 1.340 min, LCMS: m/z = 490 [M + 1]. ¹H NMR (300 MHz, DMSO-d6) δ 10.85 (s, 1H), 9.43 (d, J = 1.5 Hz, 1H), 8.46 (s, 1H), 8.35 (s, 1H), 8.14 (s, 1H), 8.07- 7.94 (m, 2H), 7.52 (d, J = 1.4 Hz, 1H), 7.33 (d, J = 1.3 Hz, 1H), 5.22-5.07 (m, 1H), 3.90 (s, 3H), 3.50-3.37 (m, 1H), 3.17 (q, J = 7.1 Hz, 3H), 2.72 (s, 1H), 1.83 (s, 3H), 1.36 (d, J = 5.8 Hz, 3H). |
| 389 390 | 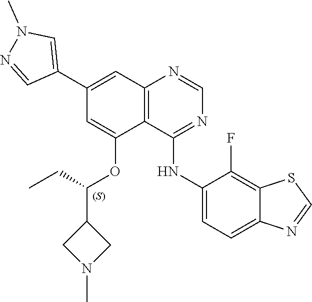 or 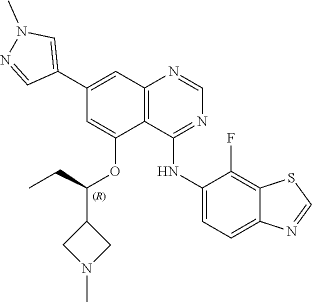 | (R)-7-fluoro-N-(7-(1-methyl-1H-pyrazol-4-yl)-5-(1-(1- methylazetidin-3-yl)propoxy)quinazolin-4-yl)benzo[d]thiazol-6- amine OR (S)-7-fluoro-N-(7-(1-methyl-1H-pyrazol-4-yl)-5-(1-(1- methylazetidin-3-yl)propoxy)quinazolin-4-yl)benzo[d]thiazol-6- amine First Eluting Peak: LC-MS: (ES, m/z): RT = 0.735 min, LCMS: m/z = 504 [M + 1]; HPLC: RT = 2.288 min; 1H NMR (400 MHz, DMSO-d6) δ 10.68 (s, 1H), 9.42 (d, J = 1.5 Hz, 1H), 8.43 (d, J = 15.0 Hz, 2H), 8.26 (t, J = 8.3 Hz, 1H), 8.13 (s, 1H), 7.99 (d, J = 8.8 Hz, 1H), 7.54 (d, J = 1.4 Hz, 1H), 7.33 (d, J = 1.6 Hz, 1H), 5.14- 5.05 (m, 1H), 3.90 (s, 3H), 2.93 (s, 1H), 1.96 (s, 3H), 1.86 (ddd, J = 14.2, 7.4, 4.9 Hz, 1H), 1.71 (dp, J = 14.0, 7.1 Hz, 1H), 0.94 (t, J = 7.4 Hz, 3H). Second Eluting Peak: LC-MS: (ES, m/z): RT = 0.741 min, LCMS: m/z = 504 [M + 1]; HPLC: RT = 3.159 min; ¹H NMR (400 MHz, DMSO-d6) δ 10.63 (s, 1H), 9.42 (d, J = 1.5 Hz, 1H), 8.43 (d, J = 10.2 Hz, 2H), 8.30 (t, J = 8.3 Hz, 1H), 8.13 (d, J = 0.8 Hz, 1H), 7.99 (d, J = 8.8 Hz, 1H), 7.54 (d, J = 1.4 Hz, 1H), 7.34 (d, J = 1.6 Hz, 1H), 5.09 (q, J = 5.9 Hz, 1H), 3.90 (s, 2H), 2.97 (s, 1H), 2.04 (s, 3H), 1.94- 1.68 (m, 2H), 0.94 (t, J = 7.4 Hz, 3H). |
| 393 | 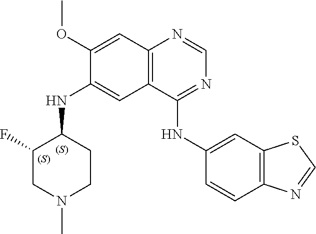 | N4-(benzo[d]thiazol-6-yl)-N6-((3S,4S)-3-fluoro-1-methylpiperidin- 4-yl)-7-methoxyquinazoline-4,6-diamine LC-MS: (ES, m/z): RT = 0.773 min, LCMS: m/z = 439 [M + 1]. ¹H NMR (300 MHz, DMSO-d6) δ 9.53 (s, 1H), 9.31 (s, 1H), 8.69 (d, J = 2.1 Hz, 1H), 8.41 (s, 1H), 8.10 (d, J = 8.8 Hz, 1H), 7.88 (dd, J = 9.0, 2.2 Hz, 1H), 7.39 (s, 1H), 7.13 (s, 1H), 5.71 (d, J = 8.7 Hz, 1H), 4.87 (d, J = 49.3 Hz, 1H), 4.02 (s, 4H), 2.94 (s, 1H), 2.45 (s, 6H), 2.11 (s, 1H), 1.67 (s, 1H). |
| 394 | 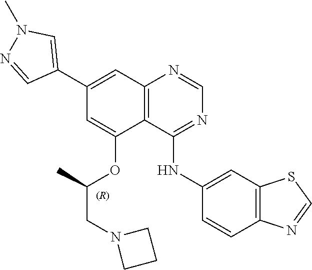 | (R)-N-(5-((1-(azetidin-1-yl)propan-2-yl)oxy)-7-(1-methyl-1H- pyrazol-4-yl)quinazolin-4-yl)benzo[d]thiazol-6-amine LC-MS: (ES, m/z): RT = 0.889 min, LCMS: m/z = 472[M + 1]. ¹H NMR (400 MHz, DMSO-d6) δ 10.77 (s, 1H), 9.33 (s, 1H), 8.93 (d, J = 2.1 Hz, 1H), 8.51 (d, J = 30.0 Hz, 2H), 8.18-8.07 (m, 2H), 7.96 (d d, J = 8.8, 2.2 Hz, 1H), 7.56 (d, J = 1.4 Hz, 1H), 7.38 (d, J = 1.6 Hz, 1H), 5.02 (s, 1H), 3.91 (s, 3H), 3.33 (s, 5H), 2.93 (s, 2H), 1.94 (s, 2H), 1.46 (d, J = 6.2 Hz, 3H). |
| 395 | 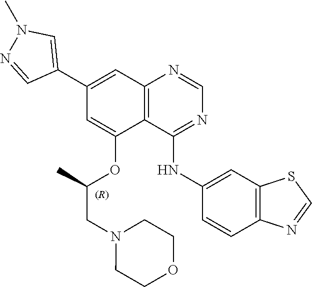 | (R)-N-(7-(1-methyl-1H-pyrazol-4-yl)-5-((1-morpholinopropan-2- yl)oxy)quinazolin-4-yl)benzo[d]thiazol-6-amine LC-MS: (ES, m/z): RT = 0.889 min, LCMS: m/z = 472[M + 1]. ¹H NMR (400 MHz, DMSO-d6) δ 10.77 (s, 1H), 9.33 (s, 1H), 8.93 (d, J = 2.1 Hz, 1H), 8.51 (d, J = 30.0 Hz, 2H), 8.18-8.07 (m, 2H), 7.96 (d d, J = 8.8, 2.2 Hz, 1H), 7.56 (d, J = 1.4 Hz, 1H), 7.38 (d, J = 1.6 Hz, 1H), 5.02 (s, 1H), 3.91 (s, 3H), 3.33 (s, 5H), 2.93 (s, 2H), 1.94 (s, 2H), 1.46 (d, J = 6.2 Hz, 3H). |
| 396 | 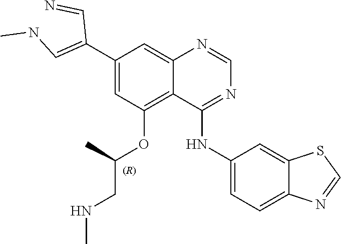 | (R)-N-(7-(1-methyl-1H-pyrazol-4-yl)-5-((1-(methylamino)propan-2- yl)oxy)quinazolin-4-yl)benzo[d]thiazol-6-amine LC-MS: (ES, m/z): RT = 0.124 min, LCMS: m/z = 446 [M + 1]; ¹H NMR (400 MHz, DMSO-d6) δ 10.74 (s, 1H), 9.31 (s, 1H), 8.88 (d, J = 2.1 Hz, 1H), 8.54 (s, 1H), 8.47 (s, 1H), 8.15 (s, 1H), 8.10 (d, J = 8.8 Hz, 1H), 7.94 (dd, J = 8.9, 2.2 Hz, 1H), 7.55 (d, J = 1.4 Hz, 1H), 7.40 (d, J = 1.4 Hz, 1H), 5.15 (s, 1H), 3.92 (s, 3H), 3.05 (s, 2H), 3.00 (s, 1H), 2.41 (s, 3H), 1.49 (d, J = 6.1 Hz, 3H), 1.24 (s, 1H). |
| 397 | 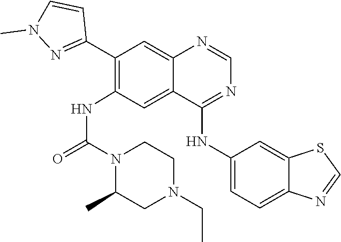 [SMILES: CCN1CCN(C(=O)Nc2cc3c(Nc4ccc5ncsc5c4)ncnc3cc2-c2ccn(C)n2)[C@H](C)C1] | (R)-N-(4-(benzo[d]thiazol-6-ylamino)-7-(1-methyl-1H-pyrazol-3- yl)quinazolin-6-yl)-4-ethyl-2-methylpiperazine-1-carboxamide LC-MS: (ES, m/z): RT = 1.122 min, LCMS: m/z = 528 [M + 1], ¹H NMR (300 MHz, DMSO-d6) δ 10.91 (s, 1H), 9.99 (s, 1H), 9.28 (s, 1H), 9.14 (s, 1H), 8.69 (d, J = 2.1 Hz, 1H), 8.52 (s, 1H), 8.20 (s, 1H), 8.06 (d, J = 8.8 Hz, 1H), 8.00-7.84 (m, 2H), 7.14 (d, J = 2.4 Hz, 1H), 4.42 (s, 1H), 3.99 (s, 4H), 3.18 (d, J = 11.5 Hz, 1H), 2.97 (d, J = 11.5 Hz, 1H), 2.85 (d, J = 11.3 Hz, 1H), 2.35 (s, 2H), 2.10 (d, J = 11.1 Hz, 1H), 1.92 (s, 1H), 1.32 (d, J = 6.6 Hz, 3H), 1.03 (t, J = 7.1 Hz, 3H). |
| 398 | 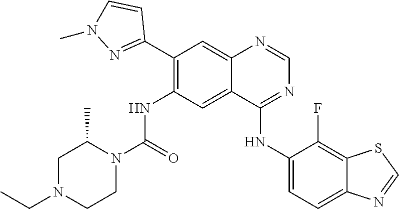 [SMILES: CCN1CCN(C(=O)Nc2cc3c(Nc4ccc5ncsc5c4F)ncnc3cc2-c2ccn(C)n2)[C@@H](C)C1] | (S)-4-ethyl-N-(4-((7-fluorobenzo[d]thiazol-6-yl)amino)-7-(1- methyl-1H-pyrazol-3-yl)quinazolin-6-yl)-2-methylpiperazine-1- carboxamide LC-MS: (ES, m/z): RT = 1.880 min, LCMS: m/z = 546 [M + 1], ¹H NMR (300 MHz, DMSO-d6) δ 10.98 (s, 1H), 10.01 (s, 1H), 9.44 (s, 1H), 9.14 (s, 1H), 8.37 (s, 1H), 8.21 (s, 1H), 7.99 (d, J = 8.9 Hz, 2H), 7.67 (t, J = 8.2 Hz, 1H), 7.15 (s, 1H), 4.41 (s, 1H), 3.99 (s, 4H), 2.96 (d, J = 11.0 Hz, 1H), 2.84 (d, J = 11.3 Hz, 1H), 2.39-2.28 (m, 2H), 2.09 (d, J = 9.4 Hz, 1H), 1.99-1.85 (m, 1H), 1.31 (d, J = 6.7 Hz, 3H), 1.03 (t, J = 7.1 Hz, 3H). |
| 399 | 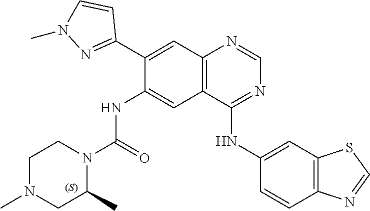 | (S)-N-(4-(benzo[d]thiazol-6-ylamino)-7-(1-methyl-1H-pyrazol-3- yl)quinazolin-6-yl)-2,4-dimethylpiperazine-1-carboxamide LC-MS: (ES, m/z): RT = 1.073 min, LCMS: m/z = 514 [M + 1], ¹H NMR (300 MHz, DMSO-d6) δ 10.95 (s, 1H), 10.03 (s, 1H), 9.31 (s, 1H), 9.16 (s, 1H), 8.71 (d, J = 2.1 Hz, 1H), 8.54 (s, 1H), 8.21 (d, J = 5.2 Hz, 2H), 8.09 (d, J = 8.8 Hz, 1H), 7.99 (d, J = 2.4 Hz, 1H), 7.93 (dd, J = 8.8, 2.1 Hz, 1H), 7.17 (d, J = 2.5 Hz, 1H), 4.43 (s, 1H), 4.01 (s, 4H), 3.31-3.14 (m, 1H), 2.89 (d, J = 11.1 Hz, 1H), 2.77 (d, J = 11.3 Hz, 1H), 2.22 (s, 3H), 2.12 (dd, J = 11.3, 3.9 Hz, 1H), 2.00- 1.86 (m, 1H), 1.34 (d, J = 6.6 Hz, 3H). |
| 400 | 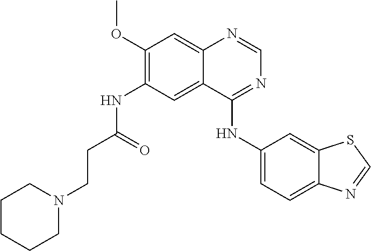 [SMILES: COc1cc2ncnc(Nc3ccc4ncsc4c3)c2cc1NC(=O)CCN1CCCCC1] | N-(4-(benzo[d]thiazol-6-ylamino)-7-methoxyquinazolin-6-yl)-3- (piperidin-1-yl)propanamide LC-MS: (ES, m/z): RT = 1.615 min, LCMS: m/z = 463.4 [M + 1], ¹H NMR (300 MHz, DMSO-d6) δ 10.74 (s, 1H), 9.89 (s, 1H), 9.28 (s, 1H), 9.05 (s, 1H), 8.65 (d, J = 2.1 Hz, 1H), 8.51 (s, 1H), 8.06 (d, J = 8.8 Hz, 1H), 7.88 (dd, J = 8.9, 2.1 Hz, 1H), 7.29 (s, 1H), 4.03 (s, 3H), 2.61 (q, J = 3.9 Hz, 3H), 1.60 (d, J = 5.7 Hz, 5H), 1.48 (s, 1H). |
| 401 | 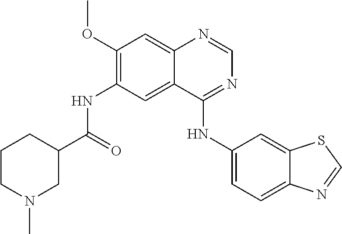 [SMILES: COc1cc2ncnc(Nc3ccc4ncsc4c3)c2cc1NC(=O)C1CCCN(C)C1] | N-(4-(benzo[d]thiazol-6-ylamino)-7-methoxyquinazolin-6-yl)-1- methylpiperidine-3-carboxamide LC-MS: (ES, m/z): RT = 1.400 min, LCMS: m/z = 449.4 [M + 1], ¹H NMR (300 MHz, DMSO-d6) δ 10.54 (s, 1H), 9.90 (s, 1H), 9.28 (s, 1H), 9.00 (s, 1H), 8.65 (d, J = 2.1 Hz, 1H), 8.52 (s, 1H), 8.06 (d, J = 8.9 Hz, 1H), 7.89 (dd, J = 8.8, 2.1 Hz, 1H), 7.27 (s, 1H), 4.03 (s, 3H), 2.72 (s, 1H), 2.50 (p, J = 1.9 Hz, 7H), 2.39 (s, 3H), 2.28 (s, 3H), 1.70 (s, 4H). |
| 402 | 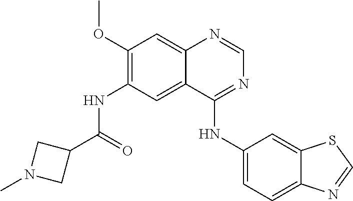 [SMILES: COc1cc2ncnc(Nc3ccc4ncsc4c3)c2cc1NC(=O)C1CN(C)C1] | N-(4-(benzo[d]thiazol-6-ylamino)-7-methoxyquinazolin-6-yl)-1- methylazetidine-3-carboxamide LC-MS: (ES, m/z): RT = 1.138 min, LCMS: m/z = 421.3 [M + 1], ¹H NMR (300 MHz, DMSO-d6) δ 9.98 (s, 1H), 9.88 (s, 1H), 9.30 (s, 1H), 8.96 (s, 1H), 8.69 (d, J = 2.1 Hz, 1H), 8.55 (s, 1H), 8.07 (d, J = 8.8 Hz, 1H), 7.91 (dd, J = 8.8, 2.1 Hz, 1H), 7.30 (s, 1H), 4.16 (d, J = 9.0 Hz, 2H), 4.01 (s, 4H), 3.97 (s, 2H), 3.90 (s, 1H), 2.75 (s, 3H). |
| 405 | 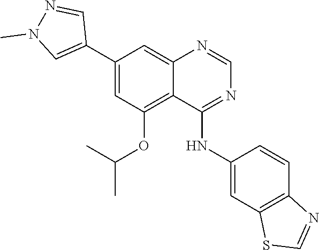 [SMILES: CC(C)Oc1cc(-c2cnn(C)c2)cc2ncnc(Nc3ccc4ncsc4c3)c12] | N-(5-isopropoxy-7-(1-methyl-1H-pyrazol-4-yl)quinazolin-4- yl)benzo[d]thiazol-6-amine LCMS: m/z = 417.1 [M + 1] |
| 406 | 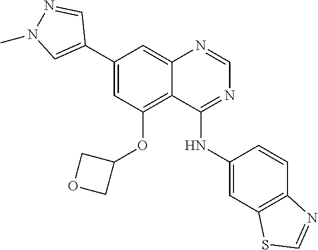 [SMILES: Cn1cc(-c2cc(OC3COC3)c3c(Nc4ccc5ncsc5c4)ncnc3c2)cn1] | N-(7-(1-methyl-1H-pyrazol-4-yl)-5-(oxetan-3-yloxy)quinazolin-4- yl)benzo[d]thiazol-6-amine LCMS: m/z = 431.1 [M + 1] |
| 407 | 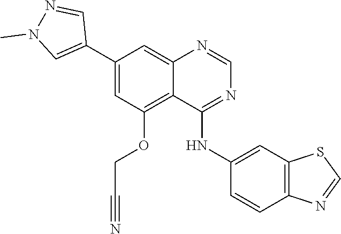 [SMILES: Cn1cc(-c2cc(OCC#N)c3c(Nc4ccc5ncsc5c4)ncnc3c2)cn1] | 2-((4-(benzo[d]thiazol-6-ylamino)-7-(1-methyl-1H-pyrazol-4- yl)quinazolin-5-yl)oxy)acetonitrile LCMS: m/z = 414.1 [M + 1] |
| 409 | 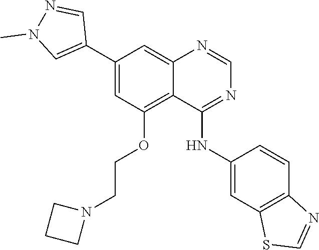 [SMILES: Cn1cc(-c2cc(OCCN3CCC3)c3c(Nc4ccc5ncsc5c4)ncnc3c2)cn1] | N-(5-(2-(azetidin-1-yl)ethoxy)-7-(1-methyl-1H-pyrazol-4- yl)quinazolin-4-yl)benzo[d]thiazol-6-amine LCMS: m/z = 458.2 [M + 1] |
| 410 | 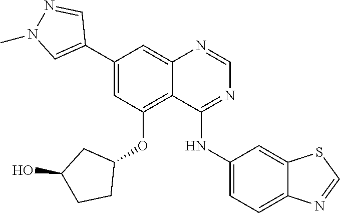 [SMILES: Cn1cc(-c2cc(O[C@@H]3CC[C@@H](O)C3)c3c(Nc4ccc5ncsc5c4)ncnc3c2)cn1] | (1R,3R)-3-((4-(benzo[d]thiazol-6-ylamino)-7-(1-methyl-1H-pyrazol- 4-yl)quinazolin-5-yl)oxy)cyclopentan-1-ol LCMS: m/z = 459.1 [M + 1] |
| 411 | 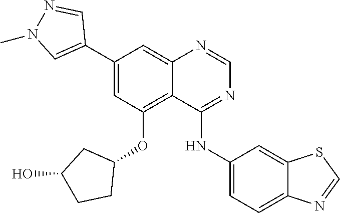 [SMILES: Cn1cc(-c2cc(O[C@@H]3CC[C@H](O)C3)c3c(Nc4ccc5ncsc5c4)ncnc3c2)cn1] | (1S,3R)-3-((4-(benzo[d]thiazol-6-ylamino)-7-(1-methyl-1H-pyrazol- 4-yl)quinazolin-5-yl)oxy)cyclopentan-1-ol LCMS: m/z = 459.2 [M + 1] |
| 412 | 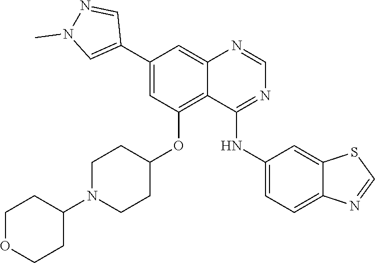 | N-(7-(1-methyl-1H-pyrazol-4-yl)-5-((1-(tetrahydro-2H-pyran-4- yl)piperidin-4-yl)oxy)quinazolin-4-yl)benzo[d]thiazol-6-amine LCMS: m/z = 452.3 [M + 1] |
| 413 | 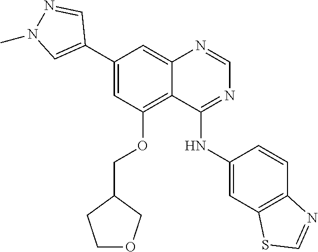 [SMILES: Cn1cc(-c2cc(OCC3CCOC3)c3c(Nc4ccc5ncsc5c4)ncnc3c2)cn1] | N-(7-(1-methyl-1H-pyrazol-4-yl)-5-((tetrahydrofuran-3- yl)methoxy)quinazolin-4-yl)benzo[d]thiazol-6-amine LCMS: m/z = 459.1 [M + 1] |
| 414 | 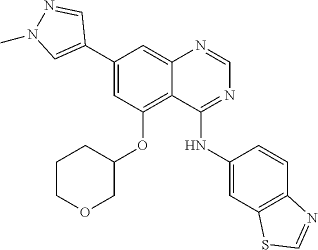 [SMILES: Cn1cc(-c2cc(OC3CCCOC3)c3c(Nc4ccc5ncsc5c4)ncnc3c2)cn1] | N-(7-(1-methyl-1H-pyrazol-4-yl)-5-((tetrahydro-2H-pyran-3- yl)oxy)quinazolin-4-yl)benzo[d]thiazol-6-amine LCMS: m/z = 459.1 [M + 1] |
| 415 | 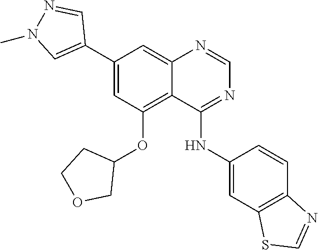 [SMILES: Cn1cc(-c2cc(OC3CCOC3)c3c(Nc4ccc5ncsc5c4)ncnc3c2)cn1] | N-(7-(1-methyl-1H-pyrazol-4-yl)-5-((tetrahydrofuran-3- yl)oxy)quinazolin-4-yl)benzo[d]thiazol-6-amine LCMS: m/z = 445.1 [M + 1] |

Chiral separation conditions

| Example # | Column | Conditions |
|---|---|---|
| 6, 7 | CHIRALPAK IC-3, | Mobile Phase A: (Hex:DCM = 1:1)(0.1% DEA):IPA = 50:50; Flow rate: |
| | 4.6*50 mm, 3 um | 1 mL/min; Gradient: 0% B to 0% B; Injection Volume: 5 ul |
| 23, 24 | CHIRALPAK IA, | Mobile Phase A: Hex:DCM = 3:1(0.5% 2M NH₃—MeOH)--HPLC, |
| | 2*25 cm, 5 μm | Mobile Phase B: EtOH--HPLC; Flow rate: 16 mL/min; Gradient: 50% |
| | | B to 50% B in 20 min; Wave Length: 220/254 nm |
| 41, 42 | CHIRALPAK IA, | Mobile Phase A: Hex:DCM = 5:1(0.5% 2M NH₃—MeOH)--HPLC, |
| | 2*25 cm, 5 μm | Mobile Phase B: MeOH--HPLC; Flow rate: 20 mL/min; Gradient: 70% |
| | | B to 70% B in 12 min; Wave Length: 220/254 nm; |
| 43, 44 | CHIRALPAK IA, | Mobile Phase A: Hex:DCM = 3:1(0.5% 2M NH₃—MeOH)--HPLC, |
| | 2*25 cm, 5 um | Mobile Phase B: IPA--HPLC; Flow rate: 20 mL/min; Gradient: 10 B to |
| | | 10 B in 20 min; 220/254 nm |
| 45, 46 | CHIRALPAK IA, | Mobile Phase A: Hex:DCM = 3:1(0.5% 2M NH₃—MeOH)--HPLC, |
| | 2*25 cm, 5 um | Mobile Phase B: IPA--HPLC; Flow rate: 20 mL/min; Gradient: 10 B to |
| | | 10 B in 20 min; 220/254 nm |
| 47, 48 | CHIRALPAK IA, | Mobile Phase A: Hex(0.5% 2M NH₃—MeOH)--HPLC, Mobile Phase |
| | 2*25 cm, 5 μm | B: EtOH--HPLC; Flow rate: 18 mL/min; Gradient: 50 B to 50 B in 14 |
| | | min; 220/254 nm |
| 52, 53 | CHIRALPAK IH, | Mobile Phase A: MTBE(0.5% 2M NH₃—MeOH)--HPLC, Mobile Phase |
| | 2*25 cm, 5 μm | B: EtOH--HPLC; Flow rate: 20 mL/min; Gradient: 20% B to 20% B in |
| | | 10 min; Wave Length: 254/220 nm |
| 55, 56 | CHIRALPAK ID, | Mobile Phase A: MTBE(0.5% 2M NH3—MeOH)--HPLC, Mobile Phase |
| | 3*25 cm, 5 μm | B: MeOH--HPLC; Flow rate: 30 mL/min; Gradient: 50% B to 50% B |
| | | in 20 min; Wave Length: 220/254 nm |
| 59, 60 | CHIRALPAK ID, | Mobile Phase A: MTBE(0.5% 2M NH₃—MeOH)--HPLC, Mobile Phase |
| | 3*25 cm, 5 μm | B: MeOH--HPLC; Flow rate: 30 mL/min; Gradient: 50% B to 50% B |
| | | in 22 min; Wave Length: 220/254 nm |
| 63, 64 | CHIRALPAK IH, | Mobile Phase A: MTBE(0.5% 2M NH₃—MeOH)--HPLC, Mobile Phase |
| | 2*25 cm, 5 μm; | B: EtOH--HPLC; Flow rate: 20 mL/min; Gradient: 20% B to 20% B in |
| | | 12 min; Wave Length: 254/220 nm |
| 70, 71 | CHIRALPAK IA, | Mobile Phase A: Hex:DCM = 3:1(0.5% 2M NH₃—MeOH)--HPLC, |
| | 2*25 cm, 5 μm; | Mobile Phase B: DCM:EtOH = 9:1--HPLC; Flow rate: 20 mL/min; |
| | | Gradient: 10% B to 10% B in 13 min; Wave Length: 220/254 nm |
| 72, 73 | CHIRALPAK IE, | Mobile Phase A: MTBE(0.5% 2M NH₃—MeOH)--HPLC, Mobile Phase |
| | 2*25 cm, 5 μm; | B: EtOH--HPLC; Flow rate: 20 mL/min; Gradient: 50% B to 50% B in |
| | | 14 min; Wave Length: 220/254 nm; |
| 74, 75 | CHIRALPAK IG, | Mobile Phase A: MTBE(0.5% 2M NH₃—MeOH)--HPLC, Mobile Phase |
| | 2*25 cm, 5 μm; | B: EtOH--HPLC; Flow rate: 18 mL/min; Gradient: 50% B to 50% B in |
| | | 21 min; Wave Length: 220/254 nm |
| 78, 79 | CHIRALPAK IH-3, | Mobile Phase A: MtBE(0.1% DEA):EtOH = 90:10; Flow rate: 1 |
| | 4.6*50 mm, 3 μm; | mL/min; Gradient: 0% B to 0% B; Injection Volume: 5 ul mL |
| 80, 81 | CHIRAL ART | Mobile Phase A: MTBE (0.5% 2M NH₃—MeOH), Mobile Phase B: |
| | Cellulose-SB, 2*25 | EtOH; Flow rate: 20 mL/min; Gradient: 20% B to 20% B in 15 min; |
| | cm, 5 μm; | Wave Length: 220/254 nm |
| 82, 83 | CHIRALPAK IH, | Mobile Phase A: MTBE(0.5% 2M NH₃—MeOH)--HPLC, Mobile Phase |
| | 2.0*25 cm, 5 um | B: EtOH--HPLC; Flow rate: 20 mL/min; Gradient: 30 B to 30 B in 8.2 |
| | | min; 254/220 nm |
| 84, 85 | CHIRAL ART | Mobile Phase A: MTBE(0.5% 2M NH₃—MeOH)--HPLC, Mobile Phase |
| | Cellulose-SB, | B: EtOH--HPLC; Flow rate: 20 mL/min; Gradient: 20 B to 20 B in 13 |
| | 2*25 cm, 5 um | min; 220/254 nm |
| 88, 89 | CHIRAL ART | Mobile Phase A: Hex(0.5% 2M NH₃—MeOH), Mobile Phase B: EtOH; |
| | Cellulose-SB, 2*25 | Flow rate: 20 mL/min; Gradient: 50% B to 50% B in 11 min; Wave |
| | cm, 5 μm; | Length: 220/254 nm |
| 92, 93 | CHIRAL ART | Mobile Phase A: MTBE(0.5% 2M NH₃—MeOH)--HPLC, Mobile Phase |
| | Cellulose-SB, | B: MeOH--HPLC; Flow rate: 20 mL/min; Gradient: 20 B to 20 B in 12 |
| | 2*25 cm, 5 um; | min; 220/254 nm |
| 94, 95 | CHIRALPAK IH, | Mobile Phase A: MTBE(0.5% 2M NH₃—MeOH)--HPLC, Mobile Phase |
| | 2*25 cm, 5 μm | B: EtOH--HPLC; Flow rate: 20 mL/min; Gradient: 10% B to 10% B in |
| | | 22 min; Wave Length: 220/254 nm; |
| 96, 97 | CHIRALPAK IH, | Mobile Phase A: MTBE(0.5%, 2M NH₃—MeOH), Mobile Phase B: |
| | 2*25 cm, 5 μm; | EtOH; Flow rate: 20 mL/min; Gradient: 50% B to 50% B in 9 min; |
| | | Wave Length: 220/254 nm |
| 98, 99 | CHIRAL ART | Mobile Phase A: Hex:DCM = 3:1(0.5% 2M NH₃—MeOH)--HPLC, |
| | Cellulose-SC, 2*25 | Mobile Phase B: EtOH--HPLC; Flow rate: 20 mL/min; Gradient: 30% |
| | cm, 5 μm; | B to 30% B in 12 min; Wave Length: 254/220 nm |
| 100, 101 | CHIRALPAK IH, | Mobile Phase A: MTBE(0.5% 2M NH₃—MeOH)--HPLC, Mobile Phase |
| | 2*25 cm, 5 μm; | B: MeOH--HPLC; Flow rate: 20 mL/min; Gradient: 10% B to 10% B |
| | | in 13 min; Wave Length: 220/254 nm |
| 102, 103 | CHIRALPAK IH, | Mobile Phase A: MTBE(0.5% 2M NH₃—MeOH)--HPLC, Mobile Phase |
| | 2*25 cm, 5 μm; | B: MeOH--HPLC; Flow rate: 20 mL/min; Gradient: 10% B to 10% B |
| | | in 13 min; Wave Length: 220/254 nm |
| 104, 105 | CHIRALPAK IH, | Mobile Phase A: MTBE(0.5% 2M NH₃—MeOH)--HPLC, Mobile Phase |
| | 2*25 cm, 5 μm; | B: EtOH--HPLC; Flow rate: 20 mL/min; Gradient: 20% B to 20% B in |
| | | 11 min; Wave Length: 254/220 nm |
| 106, 107 | CHIRALPAK IH, | Mobile Phase A: MTBE(0.5% 2M NH₃—MeOH)--HPLC, Mobile Phase |
| | 2*25 cm, 5 μm; | B: EtOH--HPLC; Flow rate: 20 mL/min; Gradient: 20% B to 20% B in |
| | | 9 min; Wave Length: 220/254 nm |
| 108, 109 | CHIRALPAK IG, | Mobile Phase A: Hex:DCM = 3:1(0.5% 2M NH₃—MeOH)--HPLC, |
| | 2*25 cm, 5 μm; | Mobile Phase B: EtOH--HPLC; Flow rate: 20 mL/min; Gradient: 20% |
| | | B to 20% B in 22 min; Wave Length: 220/254 nm |
| 110, 111 | CHIRALPAK IG, | Mobile Phase A: Hex:DCM = 3:1(0.5% 2M NH₃—MeOH)--HPLC, |
| | 2*25 cm, 5 μm; | Mobile Phase B: EtOH--HPLC; Flow rate: 20 mL/min; Gradient: 20% |
| | | B to 20% B in 22 min; Wave Length: 220/254 nm |
| 116, 117 | CHIRALPAK IH, | Mobile Phase A: MTBE(0.5% 2M NH₃—MeOH)--HPLC, Mobile Phase |
| | 2*25 cm, 5 μm; | B: EtOH--HPLC; Flow rate: 20 mL/min; Gradient: 20% B to 20% B in |
| | | 8.5 min; Wave Length: 254/220 nm |
| 118, 119 | CHIRALPAK IE, | Mobile Phase A: MTBE(0.5% 2M NH₃—MeOH)--HPLC, Mobile Phase |
| | 2*25 cm, 5 μm; | B: EtOH--HPLC; Flow rate: 20 mL/min; Gradient: 20% B to 20% B in |
| | | 13 min; Wave Length: 254/220 nm |
| 120, 121 | CHIRAL ART | Mobile Phase A: MTBE (0.5% 2M NH₃—MeOH), Mobile Phase B: |
| | Cellulose-SB, 2*25 | MeOH; Flow rate: 20 mL/min; Gradient: 15% B to 15% B in 16 min; |
| | cm, 5 μm; | Wave Length: 220/254 nm |
| 122, 123 | CHIRALPAK IH, | Mobile Phase A: MTBE (0.5% 2M NH₃—MeOH), Mobile Phase B: |
| | 3*25 cm, 5 μm; | EtOH; Flow rate: 20 mL/min; Gradient: 15% B to 15% B in 11 min; |
| | | Wave Length: 220/254 nm |
| 127, 128 | CHIRALPAK IE, | Mobile Phase A: MTBE(0.5% 2M NH₃—MeOH)--HPLC, Mobile Phase |
| | 3*25 cm, 5 μm; | B: EtOH--HPLC; Flow rate: 20 mL/min; Gradient: 25% B to 25% B in |
| | | 12.5 min; Wave Length: 220/254 nm |
| 137, 138 | CHIRAL ART | Mobile Phase A: MTBE(0.5% 2M NH₃—MeOH)--HPLC, Mobile Phase |
| | Cellulose-SB, | B: EtOH--HPLC; Flow rate: 20 mL/min; Gradient: 20 B to 20 B in 13 |
| | 2*25 cm, 5 um; | min; 220/254 nm |
| 142, 143 | CHIRALPAK ID, | Mobile Phase A: MTBE(0.5% 2M NH3—MeOH)--HPLC, Mobile Phase |
| | 2*25 cm, 5 μm; | B: EtOH--HPLC; Flow rate: 40 mL/min; Gradient: 20 B to 20 B in 15 |
| | | min; 220/254 nm |
| 144, 145 | CHIRALPAK IH, | Mobile Phase A: MTBE(0.5% 2M NH₃—MeOH)--HPLC, Mobile Phase |
| | 2*25 cm, 5 μm; | B: EtOH--HPLC; Flow rate: 20 mL/min; Gradient: 10% B to 10% B in |
| | | 11 min; Wave Length: 254/220 nm |
| 147, 148 | CHIRALPAK IG, | Mobile Phase A: Hex:DCM = 3:1(0.5% 2M NH₃—MeOH)--HPLC, |
| | 2*25 cm, 5 μm; | Mobile Phase B: MeOH--HPLC; Flow rate: 20 mL/min; Gradient: 30% |
| | | B to 30% B in 21 min; Wave Length: 220/254 nm |
| 149, 150 | CHIRALPAK IH, | Mobile Phase A: MTBE(0.5% 2M NH₃—MeOH)--HPLC, Mobile Phase |
| | 2*25 cm, 5 μm; | B: EtOH--HPLC; Flow rate: 20 mL/min; Gradient: 20% B to 20% B in |
| | | 10 min; Wave Length: 220/254 nm |
| 151, 152 | CHIRAL ART | Mobile Phase A: MTBE(0.5% 2M NH₃—MeOH)--HPLC, Mobile Phase |
| | Cellulose-SB, 2*25 | B: EtOH--HPLC; Flow rate: 20 mL/min; Gradient: 30% B to 30% B in |
| | cm, 5 μm; | 12 min; Wave Length: 220/254 nm |
| 153-154 | CHIRALPAK IH, | Mobile Phase A: MTBE(0.5% 2M NH₃—MeOH)--HPLC, Mobile Phase |
| | 2*25 cm, 5 μm; | B: EtOH--HPLC; Flow rate: 20 mL/min; Gradient: 10% B to 10% B in |
| | | 18 min; Wave Length: 254/220 nm |
| 155, 156 | CHIRALPAK IA, | Mobile Phase A: MTBE(0.5% 2M NH₃—MeOH)--HPLC, Mobile Phase |
| | 2*25 cm, 5 μm; | B: EtOH--HPLC; Flow rate: 18 mL/min; Gradient: 10% B to 10% B in |
| | | 18 min; Wave Length: 220/254 nm |
| 157, 158 | CHIRALPAK ID, | Mobile Phase A: MTBE(0.5% 2M NH₃—MeOH)--HPLC, Mobile Phase |
| | 2*25 cm, 5 μm; | B: EtOH--HPLC; Flow rate: 15 mL/min; Gradient: 50% B to 50% B in |
| | | 19 min; Wave Length: 220/254 nm |
| 159, 160 | CHIRALPAK IH, | Mobile Phase A: MTBE(0.5% 2M NH₃—MeOH)--HPLC, Mobile Phase |
| | 2*25 cm, 5 μm; | B: EtOH--HPLC; Flow rate: 20 mL/min; Gradient: 10% B to 10% B in |
| | | 12 min; Wave Length: 220/254 nm |
| 161, 162 | Xselect CSH OBD | Mobile Phase A: Water(0.1% FA), Mobile Phase B: ACN; Flow rate: 60 |
| | 30*150 mm 5 um, | mL/min; Gradient: 5% B to 15% B in 10 min, 15% B; Wave Length: |
| | | 254/220 nm |
| 166, 167 | CHIRALPAK IA, | Mobile Phase A: Hex:DCM = 3:1(0.5% 2M NH₃—MeOH)--HPLC, |
| | 2* 25 cm, 5 um; | Mobile Phase B: EtOH--HPLC; Flow rate: 18 mL/min; Gradient: 30 B to |
| | | 30 B in 16 min; 220/254 nm |
| 170, 171 | CHIRALPAK ID, | Mobile Phase A: Hex:DCM = 3:1(0.5% 2M NH₃—MeOH)--HPLC, |
| | 2*25 cm, 5 μm; | Mobile Phase B: EtOH--HPLC; Flow rate: 20 mL/min; Gradient: 10% |
| | | B to 10% B in 16 min; Wave Length: 220/254 nm |
| 172, 173 | CHIRALPAK IH, 30* | Mobile Phase A: CO₂, Mobile Phase B: MEOH(0.1% 2M NH₃—MEOH); |
| | 250 mm, 5 um; | Flow rate: 70 mL/min; Gradient: 40% B; 254 nm |
| 174, 175 | CHIRALPAK IE, | Mobile Phase A: MTBE(0.5% 2M NH₃—MeOH)--HPLC, Mobile Phase |
| | 2*25 cm, 5 um; | B: EtOH--HPLC; Flow rate: 20 mL/min; Gradient: 30 B to 30 B in 13.5 |
| | | min; 220/254 nm |
| 178, 179 | CHIRALPAK IG, | Mobile Phase A: Hex(0.5% 2M NH₃—MeOH)--HPLC, Mobile Phase B: |
| | 2*25 cm, 5 μm; | EtOH--HPLC; Flow rate: 20 mL/min; Gradient: 50% B to 50% B in 14 |
| | | min; Wave Length: 254/220 nm |
| 205, 206 | CHIRALPAK IA, | Mobile Phase A: MTBE(0.5% 2M NH₃—MeOH)--HPLC, Mobile Phase |
| | 2*25 cm, 5 μm; | B: EtOH--HPLC; Flow rate: 20 mL/min; Gradient: 10% B to 10% B in |
| | | 20 min; Wave Length: 254/220 nm |
| 207, 208 | CHIRAL ART | Mobile Phase A: Hex(0.5% 2M NH₃—MeOH)--HPLC, Mobile Phase B: |
| | Cellulose-SC, 2*25 | EtOH--HPLC; Flow rate: 20 mL/min; Gradient: 50% B to 50% B in 28 |
| | cm, 5 μm; | min; Wave Length: 220/254 nm |
| 209, 210 | Lux 5 um Celluloes-3, | Mobile Phase A: Hex(0.5% 2M NH₃—MeOH)--HPLC, Mobile Phase B: |
| | 2.12*25 cm, 5 μm; | EtOH--HPLC; Flow rate: 20 mL/min; Gradient: 20% B to 20% B in 25 |
| | | min; Wave Length: 220/254 nm |
| 211, 212 | CHIRALPAK IE, | Mobile Phase A: MTBE(0.5% 2M NH₃—MeOH)--HPLC, Mobile Phase |
| | 2*25 cm, 5 μm; | B: EtOH--HPLC; Flow rate: 20 mL/min; Gradient: 10% B to 10% B in |
| | | 14.4 min; Wave Length: 254/220 nm |
| 279, 280 | CHIRALPAK ID, | Mobile Phase A: Hex:DCM = 3:1 (0.5% 2 M NH₃—MeOH)--HPLC, |
| | 2 * 25 cm, 5 μm; | Mobile Phase B: EtOH--HPLC; Flow rate: 20 mL/min; Gradient: 50% |
| | | B to 50% B in 16 min; Wave Length: 220/254 nm |
| 282, 283 | CHIRALPAK IF, | Mobile Phase A: Hex:DCM = 3:1 (0.5% 2M NH₃—MeOH)--HPLC, |
| | 2 * 25 cm, 5 μm; | Mobile Phase B: MeOH--HPLC; Flow rate: 20 mL/min; Gradient: 20% |
| | | B to 20% B in 18 min; Wave Length: 220/254 nm |
| 287, 288 | CHIRALPAK ID, | Mobile Phase A: Hex:DCM = 3:1 (0.5% 2M NH₃—MeOH)--HPLC, |
| | 2 * 25 cm, 5 μm; | Mobile Phase B: EtOH--HPLC; Flow rate: 18 mL/min; Gradient: 50% |
| | | B to 50% B in 14 min; Wave Length: 220/254 nm |
| 289, 290 | CHIRALPAK IE, | Mobile Phase A: Hex:DCM = 3:1(0.5% 2M NH₃—MeOH)--HPLC, |
| | 2*25 cm, 5 μm; | Mobile Phase B: EtOH--HPLC; Flow rate: 20 mL/min; Gradient: 30% |
| | | B to 30% B in 14 min; Wave Length: 220/254 nm |
| 291, 292 | CHIRALPAK IE, | Mobile Phase A: MTBE(0.5% 2M NH₃—MeOH)--HPLC, Mobile Phase |
| | 3*25 cm, 5 μm; | B: EtOH--HPLC; Flow rate: 20 mL/min; Gradient: 25% B to 25% B in |
| | | 12.5 min; Wave Length: 220/254 nm |
| 293, 294 | CHIRALPAK IG, | Mobile Phase A: Hex:DCM = 3:1(0.5% 2M NH₃—MeOH)--HPLC, |
| | 2*25 cm, 5 μm; | Mobile Phase B: EtOH--HPLC; Flow rate: 20 mL/min; Gradient: 10% |
| | | B to 10% B in 33 min; Wave Length: 220/254 nm |
| 295, 296 | CHIRALPAK IH, | Mobile Phase A: MTBE(0.5% 2M NH₃—MeOH)--HPLC, Mobile Phase |
| | 2*25 cm, 5 μm; | B: EtOH--HPLC; Flow rate: 20 mL/min; Gradient: 10% B to 10% B in |
| | | 11 min; Wave Length: 254/220 nm |
| 297, 298 | CHIRALPAK ID, | Mobile Phase A: Hex:DCM = 3:1(0.5% 2M NH₃—MeOH)--HPLC, |
| | 2*25 cm, 5 μm; | Mobile Phase B: EtOH--HPLC; Flow rate: 20 mL/min; Gradient: 20% |
| | | B to 20% B in 25 min; Wave Length: 254/220 nm |
| 299, 300 | CHIRALPAK ID, | Mobile Phase A: MTBE(0.5% 2M NH₃—MeOH)--HPLC, Mobile Phase |
| | 3*25 cm, 5 μm; | B: MeOH--HPLC; Flow rate: 40 mL/min; Gradient: 30% B to 30% B |
| | | in 15 min; Wave Length: 220/254 nm |
| 321, 322 | CHIRALPAK IA, | Mobile Phase A: Hex:DCM = 3:1(0.5% 2M NH₃—MeOH)--HPLC, |
| | 2*25 cm, 20 μm; | Mobile Phase B: EtOH--HPLC; Flow rate: 20 mL/min; Gradient: 5% B |
| | | to 5% B in 20 min; Wave Length: 220/254 nm |
| 325, 326 | CHIRALPAK IA-3, | Mobile Phase A: (Hex:DCM = 3:1)(0.1% DEA ):EtOH = 70:30; Flow |
| | 4.6*50 mm; 3 um; | rate: 1 mL/min; Gradient: 0% B to 0% B; Injection Volume: 5 ul mL |
| 327, 328 | CHIRALPAK IG-3, | Mobile Phase A: (Hex:DCM = 3:1)( 0.1% DEA):EtOH = 50:50; Flow |
| | 4.6*50 mm, 3 um; | rate: 1 mL/min; Gradient: 0% B to 0% B; Injection Volume: 5 ul mL |
| 332, 333 | CHIRALPAK IA, | Mobile Phase A: Hex:DCM = 3:1(0.5% 2M NH₃—MeOH)--HPLC, |
| | 3*25 cm, 5 μm; | Mobile Phase B: EtOH--HPLC; Flow rate: 40 mL/min; Gradient: 10% |
| | | B to 10% B in 15 min; Wave Length: 220/254 nm |
| 334, 335 | CHIRALPAK IF, | Mobile Phase A: MTBE(0.5% 2M NH₃—MeOH)--HPLC, Mobile Phase |
| | 2*25 cm, 5 μm; | B: MeOH--HPLC; Flow rate: 16 mL/min; Gradient: 20% B to 20% B |
| | | in 25 min; Wave Length: 220/254 nm |
| 336, 337 | CHIRALPAK AD-H, | Mobile Phase A: Hex(0.5% 2M NH₃—MeOH)--HPLC, Mobile Phase B: |
| | 2*25 cm, 5 μm; | EtOH--HPLC; Flow rate: 18 mL/min; Gradient: 50% B to 50% B in 27 |
| | | min; Wave Length: 220/254 nm |
| 340, 341 | CHIRAL ART | Mobile Phase A: Hex:DCM = 3:1(0.5% 2M NH₃—MeOH)--HPLC, |
| | Cellulose-SB, | Mobile Phase B: IPA--HPLC; Flow rate: 20 mL/min; Gradient: 10 B to |
| | 3*25 cm, 5 um; | 10 B in 11.4 min; Wave Length: 220/254 nm |
| 344, 345 | CHIRALPAK IA, | Mobile Phase A: Hex:DCM = 3:1(0.5% 2M NH₃—MeOH)--HPLC, |
| | 2*25 cm, 5 um; | Mobile Phase B: IPA--HPLC; Flow rate: 20 mL/min; Gradient: 15 B to |
| | | 15 B in 12 min; Wave Length: 220/254 nm |
| 346, 347 | CHIRALPAK IF, | Mobile Phase A: Hex:DCM = 3:1(0.5% 2M NH₃—MeOH)--HPLC, |
| | 2*25 cm, 5 um; | Mobile Phase B: MeOH--HPLC; Flow rate: 20 mL/min; Gradient: 15 B |
| | | to 15 B in 22 min; Wave Length: 220/254 nm |
| 353, 354 | CHIRALPAK IH, | Mobile Phase A: MTBE(0.5% 2M NH₃—MeOH)--HPLC, Mobile Phase |
| | 2*25 cm, 5 μm; | B: EtOH--HPLC; Flow rate: 20 mL/min; Gradient: 20% B to 20% B in |
| | | 14 min; Wave Length: 220/254 nm |
| 358, 359 | CHIRALPAK IG, | Mobile Phase A: Hex:DCM = 3:1(0.5% 2M NH₃—MeOH)--HPLC, |
| | 3*25 cm, 5 um; | Mobile Phase B: MeOH--HPLC; Flow rate: 45 mL/min; Gradient: 20 B |
| | | to 20 B in 15 min; Wave Length: 220/254 nm |
| 360, 361 | CHIRALPAK IG, | Mobile Phase A: Hex:DCM = 3:1 (0.5% 2M NH₃—MeOH)--HPLC, |
| | 2*25 cm, 5 um; | Mobile Phase B: EtOH--HPLC; Flow rate: 20 mL/min; Gradient: 30 B to |
| | | 30 B in 19 min; 220/254 nm |
| 368, 369 | XBridge Prep OBD | Mobile Phase A: Water(10 MMOL/L NH₄HCO₃ + 0.1% NH₃•H₂O), |
| | C18 Column, | Mobile Phase B: ACN; Flow rate: 60 mL/min; Gradient: 60 B to 80 B in |
| | 30 × 150 mm 5 um; | 7 min; 254/220 nm |
| 373, 374 | CHIRALPAK IG-3, | Mobile Phase A: (Hex:DCM = 3:1)(0.1% DEA):MeOH = 92:8; Flow |
| | 4.6*50 mm, 3 um; | rate: 1 mL/min) |
| 377, 378 | CHIRALPAK IG, | Mobile Phase A: Hex:DCM = 3:1(0.5% 2M NH3—MeOH)--HPLC, |
| | 2*25 cm, 5 μm; | Mobile Phase B: IPA--HPLC; Flow rate: 18 mL/min; Gradient: 50% B |
| | | to 50% B in 14 min; Wave Length: 220/254 nm |
| 380, 381 | CHIRAL ART | Mobile Phase A: Hex:DCM = 3:1(0.5% 2M NH3—MeOH)--HPLC, |
| | Cellulose-SB, | Mobile Phase B: IPA--HPLC; Flow rate: 20 mL/min; Gradient: 10% B |
| | 2*25 cm, 5 μm; | to 10% B in 16 min; Wave Length: 220/254 nm |
| 384, 385 | CHIRALPAK IG, | Mobile Phase A: Hex:DCM = 3:1(0.5% 2M NH3—MeOH)--HPLC, |
| | 2*25 cm, 5 μm; | Mobile Phase B: IPA--HPLC; Flow rate: 18 mL/min; Gradient: 50% B |
| | | to 50% B in 14 min; Wave Length: 220/254 nm |
| 387, 388 | CHIRALPAK IA, | Mobile Phase A: Hex:DCM = 3:1(0.5% 2M NH₃—MeOH)--HPLC, |
| | 2*25 cm, 5 um; | Mobile Phase B: IPA--HPLC; Flow rate: 20 mL/min; Gradient: 15 B to |
| | | 15 B in 12 min; 220/254 nm |
| 389, 390 | CHIRALPAK IE-3, | Mobile Phase: MtBE(0.1% DEA):EtOH = 80:20, Flow: 1.0 mL/min, |
| | Column Size: | Temperature: 25° C.), Mobile Phase B: MeOH--HPLC; Flow rate: 60 |
| | 4.6*50 mm, 3 μm, | mL/min; Gradient: 50 B to 80 B in 8 min, 254; 220 nm |

### Experimental Data

### Biological Example 1. Biochemical EGFR Inhibition Assays

Inhibitory effects of the compounds of the disclosure were measured in biochemical assays that measure the phosphorylation activity of EGFR enzyme phosphorylates 2.5 micromolar 5-FAM-EEPLYWSFPAKKK-CONH₂ peptide substrate (ProfilerPro Kinase Peptide Substrate 22, PerkinElmer, Part #760366) in the presence of adenosine-5′-triphosphate (ATP) and varying concentrations of the test compound in 100 mM 2-[4-(2-hydroxyethyl)piperazin-1-yl]ethanesulfonic acid (HEPES), pH 7.5, 10 mM MgCl₂, 0.015% Brij-35, 1 mM dithiothreitol (DTT), 1.0% dimehylsulfoxide (DMSO). Assays were performed at 1.0 mM ATP or at ATP Km of the EGFR enzymes. Reactions proceeded until between 10% to 20% total peptides were phosphorylated at room temperature (25° C.) and were terminated with 35 mM 2,2′,2″,2′″-(ethane-1,2-diyldinitrilo)tetraacetic acid (EDTA). Product was detected using the Caliper mobility shift detection method where the phosphorylated peptide (product) and substrate were electrophoretically separated and measured. Percent activity was plotted against log concentration of compound and points to generate an apparent IC₅₀. The following enzyme forms of
EGFR were examples that were used in these assays:
EGFR WT (SignalChem, E10-112G)
EGFR L858R (SignalChem, E10-122BG)
EGFR (d746-750) (SignalChem, E10-122JG)
EGFR L858R C797S (SignalChem, E10-122ZG)
EGFR (d746-750) C797S (SignalChem, E10-122TG)

### Biological Example 2. Cellular EGFR Inhibition Assays

PC-9/A431 pEGFR AlphaLISA Assays

Inhibitory effects of compounds were evaluated in cellular assays that measure level of intracellular phosphorylation of EGFR in PC-9 (ECACC, #90071810, Milipore/Sigma) and A431 cell lines (ATCC, CRL-1555) using AlphaLISA sureFire ultra p-EGFR (Tyr1068) assay kit (PerkinElmer, ALSU-PEGFR-A50K). PC-9 cells were seeded at 3.125×10\hat{ }5 cells/ml in 40 μL phenol-free DMEM supplemented with 10% FBS per well of a 384 well plate (Corning, 3764), while A431 were seeded at 3.125×10≡cells/ml in 40 μL in phenol-free DMEM with 0.5% FBS. Cells were allowed to adhere overnight at 37° C./5% CO₂. On the next day, compounds were transferred at 4-fold, 10-point serial dilution from compound source plate to cell plates using liquid handler Echo550 and were incubated at 37° C./5% CO₂ for 4 hours. A431 cells were stimulated prior harvesting for 10 min with EGF at final concentration of 30 ng/ml in the incubator. Medium was removed from the plates and cells were lysed with 10 μL of 1× AlphaLISA lysis buffer (supplemented with 1× protease/phosphatase inhibitor cocktail) followed by shaking at 600 rpm for 30 minutes at room temperature. Lysates were transferred to Optiplate (Apricot designs) and 5 ul of 1× acceptor bead mix (prepared just before use) was added to each well followed by incubation at room temperature for 1.5-2 h in dark. Then 5 μL of freshly-made donor bead mix is added to each well under subdued lighting or green filters, was mixed well on the shaker and the plate was sealed and left for an overnight incubation at room temperature in dark. On the next day, the plate was read the Envision using standard AlphaLisa settings. Percent of pEGFR inhibition was plotted against log concentration of compounds to generate IC₅₀ values.

Biological assay data of the test compounds are provided in Table 2 below. For inhibitory activity against EGFR L858R C797S and EGFR (d746-750) C797S mutants, and for inhibition of phosphorylation of mutant EGFR in cells the following designations are used: ≤10 nM=A; 10.1-50 nM=B; and >50.1 nm=C.

TABLE 7

| | | Enz | |
|---|---|---|---|
| | Enz | EGFR | Phos |
| | EGFR | Exon | EGFR |
| | L858R | 19-del | PC9 |
| Ex | IC50 | IC50 | IC50 |
| 1 | A | A | A |
| 2 | A | A | A |
| 3 | A | A | B |
| 4 | B | | B |
| 5 | A | A | A |
| 6 | A | A | A |
| 7 | A | A | B |
| 8 | B | A | B |
| 9 | A | A | B |
| 10 | A | A | B |
| 11 | A | A | A |
| 12 | A | A | A |
| 13 | A | A | A |
| 14 | A | A | B |
| 15 | A | A | A |
| 16 | B | | |
| 18 | A | A | A |
| 19 | A | A | A |
| 20 | A | A | A |
| 21 | A | A | A |
| 22 | A | A | A |
| 23 | A | A | A |
| 24 | A | A | B |
| 25 | A | A | B |
| 26 | A | A | A |
| 27 | B | B | B |
| 28 | A | A | B |
| 29 | A | A | B |
| 30 | A | A | B |
| 31 | A | A | B |
| 32 | A | A | B |
| 33 | A | A | B |
| 34 | B | A | B |
| 35 | B | A | B |
| 36 | A | A | A |
| 37 | A | A | B |
| 38 | A | A | A |
| 41 | A | A | B |
| 42 | A | A | B |
| 43 | A | A | B |
| 44 | A | A | B |
| 45 | A | A | B |
| 46 | A | A | A |
| 47 | A | A | B |
| 48 | A | A | A |
| 49 | A | A | B |
| 50 | A | A | B |
| 51 | A | A | A |
| 52 | A | A | A |
| 53 | A | A | A |
| 54 | A | A | B |
| 55 | A | A | B |
| 56 | A | A | B |
| 57 | A | A | A |
| 58 | A | A | A |
| 59 | A | A | A |
| 60 | A | A | B |
| 61 | A | A | B |
| 62 | A | A | A |
| 63 | A | A | A |
| 64 | A | A | B |
| 65 | A | A | A |
| 66 | A | A | A |
| 67 | A | A | B |
| 68 | A | A | A |
| 69 | A | A | A |
| 70 | A | A | A |
| 71 | A | A | A |
| 72 | A | A | A |
| 73 | A | A | B |
| 74 | A | A | B |
| 75 | A | A | A |
| 76 | A | A | B |
| 77 | A | A | A |
| 78 | A | A | A |
| 79 | A | A | A |
| 80 | A | A | A |
| 81 | A | A | B |
| 82 | A | A | B |
| 83 | A | A | A |
| 84 | A | A | A |
| 85 | A | A | B |
| 86 | A | A | A |
| 87 | A | A | A |
| 88 | A | A | A |
| 89 | A | A | A |
| 90 | A | A | B |
| 91 | A | A | A |
| 92 | A | A | A |
| 93 | A | A | A |
| 94 | A | A | B |
| 95 | B | A | B |
| 96 | A | A | B |
| 97 | B | A | B |
| 98 | A | A | B |
| 99 | B | A | B |
| 100 | B | A | B |
| 101 | B | B | B |
| 102 | A | A | A |
| 103 | B | A | A |
| 104 | A | A | B |
| 105 | B | A | B |
| 106 | A | A | B |
| 107 | B | A | B |
| 108 | A | A | A |
| 109 | A | A | A |
| 110 | A | A | B |
| 111 | A | A | A |
| 112 | A | A | A |
| 113 | A | A | A |
| 114 | A | A | B |
| 115 | A | A | A |
| 116 | C | B | C |
| 117 | B | B | B |
| 118 | A | A | A |
| 119 | A | A | A |
| 120 | A | A | A |
| 121 | A | A | B |
| 122 | A | A | A |
| 123 | A | A | A |
| 124 | A | A | B |
| 125 | A | A | B |
| 126 | A | A | A |
| 127 | A | A | A |
| 128 | A | A | A |
| 129 | A | A | A |
| 130 | A | A | A |
| 131 | B | A | B |
| 132 | A | A | A |
| 133 | A | A | A |
| 134 | A | A | B |
| 135 | B | B | C |
| 136 | A | A | B |
| 139 | A | A | A |
| 140 | A | A | A |
| 141 | B | A | B |
| 143 | A | A | A |
| 144 | B | A | B |
| 145 | A | A | A |
| 146 | B | A | B |
| 147 | B | A | B |
| 148 | A | A | B |
| 149 | C | B | B |
| 150 | B | A | A |
| 151 | A | A | A |
| 152 | A | A | A |
| 153 | A | A | B |
| 154 | A | A | A |
| 155 | A | A | A |
| 156 | A | A | A |
| 157 | A | A | A |
| 158 | A | A | B |
| 159 | B | A | B |
| 160 | A | A | A |
| 161 | A | A | A |
| 162 | A | A | B |
| 163 | A | A | A |
| 164 | A | A | B |
| 165 | A | A | A |
| 166 | A | A | A |
| 167 | B | A | B |
| 168 | A | A | B |
| 169 | A | A | B |
| 170 | A | A | A |
| 171 | A | A | A |
| 172 | A | A | B |
| 173 | A | A | A |
| 174 | A | A | A |
| 175 | A | A | B |
| 176 | B | A | A |
| 177 | A | A | A |
| 178 | A | A | A |
| 179 | C | B | C |
| 180 | A | A | A |
| 181 | A | A | A |
| 182 | A | A | A |
| 183 | B | A | B |
| 184 | A | A | A |
| 185 | A | A | A |
| 186 | A | A | A |
| 187 | B | A | B |
| 188 | B | A | B |
| 189 | B | A | B |
| 190 | A | A | A |
| 191 | A | A | B |
| 192 | A | A | B |
| 193 | A | A | A |
| 194 | A | A | A |
| 195 | A | A | B |
| 196 | A | A | B |
| 197 | A | A | A |
| 198 | A | A | A |
| 199 | A | A | B |
| 200 | C | C | A |
| 201 | A | A | B |
| 202 | A | A | A |
| 203 | B | A | B |
| 204 | A | A | A |
| 205 | A | A | B |
| 206 | A | A | B |
| 207 | A | A | A |
| 208 | A | A | A |
| 209 | A | A | A |
| 210 | A | A | A |
| 211 | C | B | C |
| 212 | B | B | C |
| 226 | C | B | |
| 227 | A | A | B |
| 240 | A | A | A |
| 241 | A | A | A |
| 242 | A | A | B |
| 243 | B | B | B |
| 245 | A | A | B |
| 246 | A | A | B |
| 247 | A | A | B |
| 248 | B | A | B |
| 249 | A | A | B |
| 250 | A | A | B |
| 251 | A | A | B |
| 252 | A | A | A |
| 253 | A | A | A |
| 254 | A | A | B |
| 255 | A | A | A |
| 256 | C | C | |
| 257 | A | A | A |
| 258 | A | A | A |
| 259 | A | A | A |
| 260 | A | A | B |
| 261 | A | A | A |
| 262 | A | A | A |
| 263 | A | A | A |
| 264 | A | A | B |
| 265 | A | | A |
| 266 | A | A | B |
| 270 | A | A | B |
| 271 | A | A | B |
| 272 | A | A | A |
| 273 | A | A | A |
| 274 | A | A | A |
| 275 | A | A | B |
| 276 | A | A | A |
| 277 | A | A | A |
| 278 | A | A | A |
| 279 | A | A | A |
| 280 | A | A | A |
| 281 | A | A | A |
| 282 | A | A | A |
| 283 | A | A | A |
| 284 | A | A | A |
| 285 | A | A | A |
| 286 | B | A | B |
| 287 | A | A | A |
| 288 | A | A | A |
| 289 | A | A | A |
| 290 | A | A | A |
| 291 | A | A | A |
| 292 | A | A | A |
| 293 | A | A | B |
| 294 | A | A | B |
| 295 | B | A | A |
| 296 | A | A | A |
| 297 | A | A | A |
| 298 | A | A | A |
| 299 | B | A | B |
| 300 | B | A | A |
| 301 | A | A | |
| 305 | A | A | A |
| 306 | A | A | A |
| 307 | A | A | A |
| 308 | B | A | B |
| 309 | A | A | A |
| 310 | A | A | A |
| 311 | A | A | A |
| 312 | A | A | B |
| 313 | A | A | B |
| 314 | A | A | A |
| 315 | A | A | A |
| 316 | B | A | B |
| 317 | A | A | A |
| 318 | A | A | A |
| 319 | B | A | A |
| 320 | A | A | B |
| 321 | B | A | B |
| 322 | B | A | B |
| 325 | B | A | C |
| 326 | A | A | A |
| 327 | A | A | A |
| 328 | A | A | A |
| 329 | A | A | A |
| 331 | A | A | A |
| 332 | A | A | A |
| 333 | A | A | B |
| 334 | A | A | B |
| 335 | A | A | A |
| 336 | A | A | A |
| 337 | A | A | A |
| 339 | A | A | A |
| 340 | A | A | A |
| 341 | A | A | A |
| 343 | A | A | A |
| 344 | A | A | A |
| 345 | A | A | A |
| 346 | A | A | A |
| 347 | A | A | A |
| 348 | A | A | A |
| 349 | A | A | A |
| 350 | A | A | A |
| 351 | A | A | A |
| 352 | A | A | A |
| 353 | A | A | A |
| 354 | B | A | B |
| 356 | A | A | A |
| 358 | A | A | A |
| 359 | A | A | B |
| 360 | A | A | A |
| 361 | A | A | A |
| 362 | A | A | A |
| 363 | A | A | A |
| 364 | A | A | A |
| 365 | A | A | A |
| 366 | B | A | A |
| 367 | B | A | B |
| 368 | A | A | B |
| 369 | B | A | B |
| 370 | A | A | A |
| 371 | B | A | B |
| 373 | A | A | A |
| 374 | A | A | B |
| 375 | B | A | B |
| 376 | A | A | B |
| 377 | B | B | B |
| 378 | A | A | A |
| 379 | B | A | C |
| 380 | A | A | B |
| 381 | A | A | B |
| 382 | B | A | C |
| 383 | A | A | B |
| 384 | B | B | B |
| 385 | A | A | A |
| 386 | A | A | A |
| 387 | A | A | A |
| 388 | C | B | C |
| 389 | B | A | B |
| 390 | C | C | C |
| 393 | A | A | A |
| 394 | A | A | A |
| 395 | A | A | B |
| 396 | B | A | A |
| 397 | A | A | A |
| 398 | A | A | A |
| 399 | A | A | A |
| 400 | B | A | B |
| 401 | B | B | B |
| 402 | C | B | B |
| 405 | A | A | A |
| 406 | A | A | A |
| 407 | A | A | B |
| 409 | B | A | B |
| 410 | A | A | A |
| 411 | A | A | A |
| 412 | B | A | B |
| 413 | A | A | B |
| 414 | A | A | A |
| 415 | A | A | B |
| A03 | B | A | B |
| A05 | A | A | A |
| A06 | A | A | A |
| A07 | A | A | B |
| A08 | A | A | B |
| A09 | A | A | A |
| A10 | A | A | A |
| A11 | C | B | |
| A16 | B | A | B |
| A17 | B | A | B |
| A18 | B | A | B |
| A19 | A | A | A |
| A20 | A | A | A |
| A22 | A | A | B |
| A23 | A | A | B |
| A24 | C | B | B |
| A25 | A | A | B |
| A26 | A | A | A |
| A27 | A | A | B |
| A28 | B | A | C |
| A29 | A | A | B |
| A30 | A | A | C |
| A31 | A | A | B |
| A32 | B | A | B |
| A33 | A | A | A |
| A34 | B | A | C |
| A35 | A | A | B |
| A36 | A | A | B |
| A37 | A | A | B |
| A38 | B | A | B |
| A39 | B | A | C |
| A40 | A | A | B |
| A41 | A | A | B |
| A42 | A | A | A |
| A43 | A | A | B |
| A44 | A | A | B |
| A45 | B | A | C |
| A47 | A | A | A |
| A48 | A | A | A |
| A49 | B | A | B |
| A50 | A | A | A |
| A51 | A | A | A |
| A52 | A | A | A |
| A53 | A | A | A |
| A54 | A | A | B |
| A55 | A | A | A |
| A56 | A | A | A |
| A57 | A | A | B |
| A58 | C | C | |
| A59 | A | A | B |
| A60 | B | A | B |

### EQUIVALENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those skilled in the art will recognize or be able to ascertain using no more than routine experimentation, many equivalents to the specific embodiments of the disclosure described and claimed herein. Such equivalents are intended to be encompassed by the following claims.

1. A compound of Formula (I): 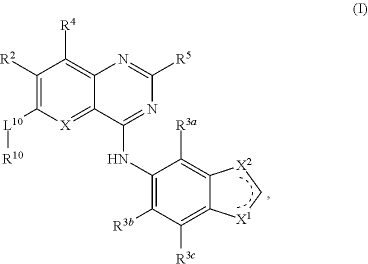
or a pharmaceutically acceptable salt thereof, wherein
X¹ is S and X² is N or X¹ is N and X² is S;
X is CR^{x} or N;
R^{x} is H, F, or —O—R¹;
R¹ is C₁-C₆ alkyl, C₃-C₆ cycloalkyl, or 4- to 12-membered heterocyclyl, wherein the alkyl, cycloalkyl, and heterocyclyl represented by R¹ are optionally substituted with 1 to 4 groups independently selected from deuterium, halo, C₁-C₄ alkyl, ═O (as valence permits), —OR^{1c}, CN, NR^{1a}R^{1b}, C₃-C₆ cycloalkyl, and 4- to 8-membered heterocyclyl, wherein the alkyl is optionally substituted with 1 to 3 groups selected from halo, deuterium, and OR^{1a} and NR^{1a}R^{1b}, and the heterocyclyl and C₃-C₆ cycloalkyl are each optionally substituted with 1 to 4 groups selected from ═O, NR^{1a}R^{1b}, and C₁-C₄ alkyl optionally substituted with NR^{1a}R^{1b},
L¹⁰-R¹⁰ is halo; or
L¹⁰ is a bond, NH, —NHC(O)—*, —NHC(O)O—*, O, or —OC(O)—*; wherein —* represents the point which attaches to R¹⁰; and
R¹⁰ is H; or
C₁-C₄ alkyl optionally substituted with 1 to 4 groups independently selected from halo, deuterium, OR^{1a}, NR^{1a}R^{1b}, C₃-C₆ cycloalkyl, 4- to 12-membered heterocyclyl and 5 to 10 membered heteroaryl, wherein the heterocyclyl and heteroaryl are each optionally substituted with 1 to 4 groups independently selected from halo, deuterium, ═O, NR^{1a}R^{1b}, and C₁-C₄ alkyl; or
C₃-C₈ cycloalkyl, phenyl, 4- to 12-membered heterocyclyl, or 5- to 12-membered heteroaryl, wherein the cycloalkyl, phenyl, heterocyclyl and heteroaryl represented by R¹⁰ are each optionally substituted with 1 to 4 groups independently selected from R¹¹;
each R¹¹ is independently selected from halo, deuterium, OR^{1a}, C(O)R^{1a}, C(O)NR^{1a}R^{1b}, NR^{1a}C(O)OR^{1a}, NR^{1a}R^{1b}, S(O)₂R^{1a}, C₁-C₄ alkyl, C₃-C₆ cycloalkyl, phenyl, 4- to 12-membered heterocyclyl and 5- to 12-membered heteroaryl, wherein the alkyl, cycloalkyl, phenyl, heterocyclyl and heteroaryl represented by R¹¹ are each optionally substituted with 1 to 4 groups selected from deuterium, halo, C₁-C₄ alkyl, OR^{1a} and NR^{1a}R^{1b}, or two R¹ which are attached to the same carbon atom are taken together to form ═O;
R² is halo, NR^{1a}R^{1b}, C₁-C₄ alkyl, C₂-C₄ alkenyl, C₂-C₄ alkynyl, C₁-C₄ alkoxy, C₂-C₄ alkenyloxy, C₂-C₄ alkynyloxy, C₃-C₈ cycloalkyl, 4- to 12-membered heterocyclyl, phenyl, or 5- or 12-membered heteroaryl, wherein the alkyl, alkenyl, alkynyl, alkoxy, alkenyloxy, alkynyloxy, cycloalkyl, heterocycyl, phenyl, and heteroaryl represented by R² are each optionally substituted with 1 to 4 groups represented by R^{2a}; and R^{2a} is selected from deuterium, halo, ═O (as valence permits), OR^{1c}, NR^{1a}R^{1b}, C(O)R^{1c}, C(O)OR^{1c}, —S(O)₂R^{1c}, C₁-C₄ alkyl, and 4- to 12-membered heterocyclyl, wherein the C₁-C₄ alkyl and C₁-C₄ alkoxy groups represented by R^{2a} are each optionally substituted with 1 to 4 groups selected from deuterium, halo, OH, and C₁-C₄ alkoxy, and the 4- to 12-membered heterocyclyl represented by R^{2a} is optionally substituted with 1 to 4 groups independently selected from halo, deuterium, ═O, NR^{1a}R^{1b}, and C₁-C₄ alkyl;
R^{1a} is H, deuterium, C₁-C₄ alkyl, or C₃-C₆ cycloalkyl;
R^{1b} is H, deuterium, C₁-C₄ alkyl, or C₃-C₆ cycloalkyl;
R^{1c} is H, deuterium, C₁-C₄ alkyl optionally substituted with 1 to 3 halo, or C₃-C₆ cycloalkyl;
R^{3a} is H, deuterium, halo, OH, C₁₋₄ alkyl, or C₁-C₄ alkoxy;
R^{3b} is H, deuterium, halo, OH, C₁₋₄ alkyl, or C₁-C₄ alkoxy;
R^{3c} is H, deuterium, halo, OH, C₁₋₄ alkyl, or C₁-C₄ alkoxy;
R⁴ is H, deuterium or halo; and
R⁵ is H or deuterium.

2. The compound of claim 1 having Formula (II): 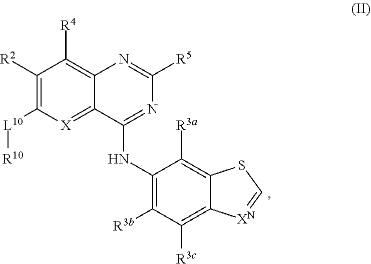
or a pharmaceutically acceptable salt thereof.

3. The compound of claim 2 having Formula (III): 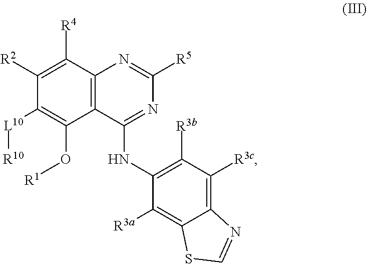
or a pharmaceutically acceptable salt thereof, wherein
L¹⁰-R¹⁰ is H or halo;
R^{3a} is H, deuterium, or halo;
R^{3b} is H, deuterium, or halo; and
R^{3c} is H, deuterium, or halo.

4. The compound of claim 1, having Formula (III-A): 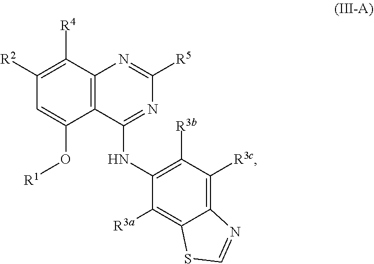
or a pharmaceutically acceptable salt thereof.

5. The compound of claim 1 or a pharmaceutically acceptable salt thereof, wherein:
(i) R¹ is C₁-C₆ alkyl, C₃-C₆ cycloalkyl, or 4- to 8-membered heterocyclyl, wherein the alkyl, cycloalkyl, and heterocyclyl represented by R¹ are optionally substituted with 1 to 4 groups independently selected from halo, CN, ═O, NR^{1a}R^{1b}, C₁-C₄ alkyl (optionally substituted with 1 to 4 groups selected from halo, —OH and C₁-C₄ alkoxy), —OH, C₁-C₄ alkoxy, NR^{1a}R^{1b}, C₃-C₆ cycloalkyl, and 4- to 8-membered heterocyclyl (optionally substituted with ═O, NR^{1a}R^{1b}, and/or C₁-C₄ alkyl);
(ii) R¹ is:
C₁-C₆ alkyl optionally substituted with 1 or 4 groups selected from halo, CN, ═O, NHCH₃, N(CH₃)₂, OH, C₁-C₄ alkoxy, C₃-C₄ cycloalkyl, and 4- to 8-membered heterocyclyl (optionally substituted with 1 to 4 groups selected from ═O, NR^{1a}R^{1b}, and/or C₁-C₄ alkyl optionally substituted with NR^{1a}R^{1b});
C₃-C₆cycloalkyl optionally substituted with 1 or 2 groups selected from C₁-C₄ alkyl, OH, NH₂, NHCH₃, and N(CH₃)₂;
4- to 6-membered heterocyclyl optionally substituted with 1 or 3 groups selected from halo, C₁-C4 alkyl (optionally substituted with 1 to 4 groups selected from halo, —OH and C₁-C₄alkoxy), ═O, OH, C₁-C₄ alkoxy, oxetanyl, and tetrahyropyranyl; or
(iii) R¹ is:
C₁-C₄ alkyl optionally substituted with 1 or 2 groups selected from N(CH₃)₂, ═O, azetidinyl, oxetanyl, and morpholinyl;
oxetanyl;
pyrrolidinyl optionally substituted with 1 or 2 groups selected from methyl and ═O; or
piperidinyl optionally substituted with F or oxetanyl.

6-7. (canceled)

8. The compound of claim 1, or a pharmaceutically acceptable salt thereof, wherein;
(i) R² is;
C₁-C₄ alkyl, C₂-C₄ alkenyl, C₂-C₄ alkynyl, C₁-C₄ alkoxy, 4- to 10-membered heterocyclyl, phenyl, or 5- or 6-membered heteroaryl,
wherein the alkyl, alkenyl, alkynyl, and alkoxy represented by R² are each optionally substituted with 1 to 4 groups selected from deuterium, halo, ═O (as valence permits), OH, NR^{1a}R^{1b}, C(O)R^{1c}, C(O)OR^{1c}, —S(O)₂R^{1c}, C₁-C₄ alkyl, C₁-C₄ alkoxy, and 4- to 6-membered heterocyclyl (optionally substituted with ═O, NR^{1a}R^{1b}, C₁-C₄ alkyl);
wherein the 4- to 10-membered heterocycyl, phenyl, and 5- or 6-heteroaryl represented by R² are each optionally substituted with 1 to 4 groups selected from deuterium, halo, ═O (as valence permits), OH, NR^{1a}R^{1b}, C(O)R^{1c}, C(O)OR^{1c}, —S(O)₂R^{1c}, C₁-C₄ alkyl (optionally substituted with 1 to 4 groups selected from halo, —OH and C₁-C₄ alkoxy), C₁-C₄ alkoxy (optionally substituted with 1 to 4 groups selected from halo and —OH), and 4- to 6-membered heterocyclyl (optionally substituted with 1 to 2 groups selected from ═O, NR^{1a}R^{1b}, C₁-C₄ alkyl);
(ii) R² is:
C₁-C₄ alkoxy optionally substituted with 1 to 4 groups selected from deuterium, halo, ═O (as valence permits), C₁-C₄ alkoxy, and 4- to 6-membered heterocyclyl (optionally substituted with ═O, NR^{1a}R^{1a}, C₁-C₄ alkyl)
C₂-C₄ alkynyl optionally substituted with 1 to 2 groups selected from C₁-C₄ alkyl and 4- to 6-membered heterocyclyl (optionally substituted with ═O, NR^{1a}R^{1b}, C₁-C₄ alkyl);
4- to 10-membered heterocyclyl, phenyl, or 5- or 6-membered heteroaryl each optionally substituted with 1 to 4 groups selected from halo, ═O (as valence permits), C₁-C₄ alkyl (optionally substituted with 1 to 4 groups selected from halo, —OH and C₁-C₄ alkoxy), C₁-C₄ alkoxy (optionally substituted with 1 to 4 groups selected from halo and —OH), —C(O)OC₁-C₄ alkyl, —S(O)₂C₁-C₄ alkyl, and 4- to 6-membered heterocyclyl (optionally substituted with 1 to 2 groups selected from ═O, NR^{1a}R^{1b}, C₁-C₄ alkyl);
(iii) R² is:
C₁-C₄ alkoxy optionally substituted with 1 to 3 groups selected from deuterium, OCH₃, and piperizinyl (optionally substituted with methyl);
C₂-C₄ alkynyl optionally substituted with 4- to 6-membered heterocyclyl (optionally substituted with C₁-C₂alkyl);
1,3-dihydro-2H-imidazol-2-onyl optionally substituted with 1 or 2 C₁-C₂alkyl;
2,5 [symbol] -diazabicyclo[4.1.0]heptanyl optionally substituted with C₁-C₂alkyl;
6-oxa-3-azabicyclo[3.1.1]heptanyl;
2-oxa-6 [symbol] -azaspiro[3.3]heptanyl;
dihydro-3H-pyrazol-3-onyl optionally substituted with 1 or 2 groups selected from C₁-C₃ alkyl (optionally substituted with 1 to 3 halo);
hexahydro-1H-2 [symbol] -pyrrolo[2,1-c]pyrazinyl;
imidazolidinyl optionally substituted with 1 or 2 groups selected from ═O and C₁-C₃ alkyl;
morpholinyl optionally substituted with 1 or 2 groups selected from C₁-C₃ alkyl;
phenyl optionally substituted with S(O)₂CH₃;
piperizinyl optionally substituted with 1 or 3 groups selected from C₁-C₃ alkyl (optionally substituted with —OH or C₁-C₂alkoxy) and —C(O)OC₁-C₄ alkyl;
pyrazolyl optionally substituted with 1 or 2 groups selected from piperizinyl (optional substituted with methyl), piperidinyl (optionally substituted with methyl), C₁-C₄ alkyl, and C₁-C₄ alkoxy, wherein the C₁-C₄ alkyl and C₁-C₄ alkoxy are each optionally substituted with 1 to 4 groups selected from halo and OH;
pyrrolidinyl optionally substituted with 1 or 2 groups selected from C₁-C₃ alkyl (optionally substituted with —OH) and C₁-C₂ alkoxy; or
triazolyl optionally substituted with C₁-C₃ alkyl; or
(iv) R² is:
pyrazolyl optional substituted with 1 or 2 groups selected from methyl, methoxy, —OCHF₂, —CH₂C(OH)(CH₃)₂, —CH(CH₃)CH₂OH, and —C(CH₃)₂CH₂OH;
pyrrolidinyl optionally substituted with 1 or 2 groups selected from methoxy and —C(OH)(CH₃)₂;
6-oxa-3-azabicyclo[3.1.1]heptanyl; or
2-oxa-6 [symbol] -azaspiro[3.3]heptanyl.

9-11. (canceled)

12. The compound of claim 2 having the Formula (IV): 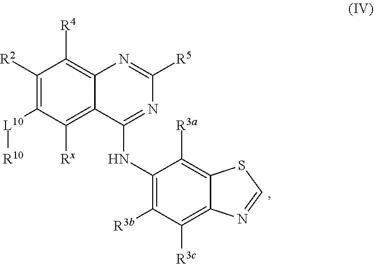
or a pharmaceutically acceptable salt thereof, wherein
R^{x} is H, F;
R^{3a} is H, deuterium, or halo;
R^{3b} is H, deuterium, or halo; and
R^{3c} is H, deuterium, or halo.

13. The compound of claim 12, or a pharmaceutically acceptable salt thereof, wherein L¹⁰ is a bond, NH, —NHC(O)—*, or O; wherein —* represents the point which attaches to R¹⁰.

14. The compound of claim 13, or a pharmaceutically acceptable salt thereof, wherein
L¹⁰-R¹⁰ is H or halo; or
R¹⁰ is:
C₁-C₄ alkyl optionally substituted with 1 to 4 groups independently selected from halo, OR^{1a}, NR^{1a}R^{1b}, 4 to 6 membered heterocyclyl wherein the 4 to 6 membered heterocyclyl is optionally substituted with 1 to 2 groups independently selected from halo, ═O, NR^{1a}R^{1b}, and C₁-C₄ alkyl; or
4- to 8-membered heterocyclyl optionally substituted with 1 to 2 groups selected from halo, ═O, C₁-C₄ alkyl, OR^{1a}, C(O)R^{1a}; C(O)NR^{1a}R^{1b}, NR^{1a}C(O)OR^{1a}, and NR^{1a}R^{1b}; or
5- to 6-membered heteroaryl optionally substituted with 1 to 2 groups selected from halo, C₁-C₄ alkyl, OR^{1a}, C(O)R^{1a}; C(O)NR^{1a}R^{1b}, NR^{1a}C(O)OR^{1a}, NR^{1a}R^{1b}, and 4- to 6-membered heterocyclyl (which is further optionally substituted with 1 or 2 groups selected from halo, ═O, NR^{1a}R^{1b}, and C₁-C₄ alkyl).

15. The compound of claim 14, or a pharmaceutically acceptable salt thereof, wherein L¹⁰-R¹⁰ is H or halo.

16. The compound of claim 14, or a pharmaceutically acceptable salt thereof, wherein:
(i) L¹⁰ is a bond; and
R¹⁰ is 5- to 6-membered heteroaryl optionally substituted with C₁-C₂alkyl or 4- to 6-membered heterocyclyl (which is further optionally substituted with 1 or 2 groups selected from halo and C₁-C₂alkyl) (e.g., pyrazolyl optionally substituted with methyl or piperidinyl (optionally substituted with 1 or 2 groups selected from F and methyl);
(ii) L¹⁰ is NH; and
R¹⁰ is 4- to 6-membered heterocyclyl optionally substituted with 1 to 2 groups selected from halo, C₁-C₂ alkyl, and —C(O)C₁-C₂ alkyl (e.g., piperidinyl optionally substituted with 1 or 2 groups selected from F, methyl, and —C(O)CH₂CH₃);
(iii) L¹⁰ is —NHC(O)—*, wherein —* represents the point which attaches to R¹⁰, and R¹⁰ is:
C₁-C₂alkyl optionally substituted with 4 to 6 membered heterocyclyl (e.g., ethyl optionally substituted with piperidinyl); or
4- to 8-membered heterocyclyl optionally substituted with 1 to 2 groups selected from halo, C₁-C₂ alkyl, and —C(O)C₁-C₂ alkyl (e.g., azetidinyl optionally substituted with methyl, piperidinyl optionally substituted with 1 or 2 groups selected from methyl and fluoro, piperizinyl optionally substituted with 1 or 2 groups selected from methyl and ethyl, octahydropyrrolo[3,4-b]pyrrolyl optionally substituted with methyl); or
(iv) L¹⁰ is O; and
R¹⁰ is:
C₁-C₂alkyl (e.g., CH₃); or
4- to 6-membered heterocyclyl optionally substituted with 1 to 2 groups selected from halo, C₁-C₂alkyl, and —C(O)C₁-C₂alkyl (e.g., tetrohydrofuranyl, tetrohydropyranyl, or piperidinyl optionally substituted with —C(O)CH₂CH₃).

17-19. (canceled)

20. The compound of claim 12, or a pharmaceutically acceptable salt thereof, wherein;
(i) R² is C₁-C₄ alkyl or 5- or 6-membered heteroaryl (e.g., 5-membered heteroaryl), wherein the heteroaryl represented by R² is optionally substituted with 1 to 4 groups selected from C₁-C₄ alkyl, C₁-C₄ alkoxy, and 4 to 6 membered heterocyclyl (optionally substituted with C₁-C₄ alkyl); or
(ii) R² is methyl or pyrazolyl optionally substituted with 1 or 2 groups selected from methyl, methoxy, and piperidinyl (optionally substituted with methyl).

21. (canceled)

22. The compound of claim 12, or a pharmaceutically acceptable salt thereof wherein R^{x} is H.

23. The compound of claim 1, or pharmaceutically acceptable salt thereof, wherein R^{3a} is H or halo; R^{3b} and R^{3c} are H; and R⁴ and R⁵ are H.

24. (canceled)

25. The compound of claim 1, or pharmaceutically acceptable salt thereof, wherein R^{1a} is H or C₁-C₄ alkyl; R^{1b} is H or C₁-C₄ alkyl; and R^{1c} is H or C₁-C₄ alkyl optionally substituted with 1 to 3 halo (F).

26. A pharmaceutical composition comprising a pharmaceutically acceptable carrier and a compound of claim 1, or a pharmaceutically acceptable salt thereof.

27. A method of treating a cancer, comprising administering a subject in need thereof an effective amount of a compound of claim 1, or a pharmaceutically acceptable salt thereof.

28. The method of claim 27, wherein the cancer is non-small cell lung cancer; or wherein the cancer in the subject in need thereof has metastasized.

29. (canceled)

30. The method of claim 27, wherein the cancer is characterized by: i) epidermal growth factor receptor EGFR L858R mutation and/or exon 19 deletion; and ii) C797S mutation or wherein the cancer is characterized by epidermal growth factor receptor (EGFR) T790M mutation.

31. (canceled)

32. The method of claim 27, further comprises administering the subject in need thereof an effective amount of afatinib or osimertinib.

33. (canceled)
